(19) United States
(12) Patent Application Publication
(10) Pub. No.: US 2003/0044783 A1
(43) Pub. Date: Mar. 6, 2003

(54) Human genes and gene expression products

(76) Inventors: Lewis T. Williams, Mill Valley, CA (US); Jaime Escobedo, Alamo, CA (US); Michael A. Innis, (US); Pablo Dominguez Garcia, San Francisco, CA (US); Julie Sudduth-Klinger, Kensington, CA (US); Christoph Reinhard, Alameda, CA (US); Filippo Randazzo, Oakland, CA (US); Giulia C. Kennedy, San Francisco, CA (US); David Pot, Arlington, VA (US); Altaf Kassam, Oakland, CA (US); George Lamson, Moraga, CA (US); Radjoe Drmanac, Palo Alto, CA (US); Mark Dickson, Hollister, CA (US); Ivan Labat, Mountain View, CA (US); Lee William Jones, Sunnyvale, CA (US); Birgit Stache-Crain, Sunnyvale, CA (US)

Correspondence Address: Chiron Corporation Intellectual Property -R440, PO Box 8097, Emeryville, CA, 94662-8097, US

(21) Appl. No.: 09/803,719

(22) Filed: Mar. 9, 2001

Related U.S. Application Data
(60) Provisional application No. 60/188,609, filed on Mar. 9, 2000.

(51) Int. Cl. C12Q 1/68; C07H 21/04; C12N 9/00; C12P 21/02; C12N 5/06; C07K 14/435; C07K 16/40

(52) U.S. Cl. 435/6; 435/69.1; 435/183; 435/320.1; 435/325; 530/350; 536/23.2; 530/388.1

(57) ABSTRACT

This invention relates to novel human polynucleotides and variants thereof, their encoded polypeptides and variants thereof, to genes corresponding to these polynucleotides and to proteins expressed by the genes. The invention also relates to diagnostic and therapeutic agents employing such novel human polynucleotides, their corresponding genes or gene products, e.g., these genes and proteins, including probes, antisense constructs, and antibodies.

### CROSS-REFERENCE TO RELATED APPLICATIONS

This application claims the benefit of U.S. provisional application serial No. 60/188,609, filed Mar. 9, 2000, which application is incorporated herein by reference in its entirety.

### FIELD OF THE INVENTION

The present invention relates to polynucleotides of human origin and the encoded gene products.

### BACKGROUND OF THE INVENTION

Identification of novel polynucleotides, particularly those that encode an expressed gene product, is important in the advancement of drug discovery, diagnostic technologies, and the understanding of the progression and nature of complex diseases such as cancer. Identification of genes expressed in different cell types isolated from sources that differ in disease state or stage, developmental stage, exposure to various environmental factors, the tissue of origin, the species from which the tissue was isolated, and the like is key to identifying the genetic factors that are responsible for the phenotypes associated with these various differences.

This invention provides novel human polynucleotides, the polypeptides encoded by these polynucleotides, and the genes and proteins corresponding to these novel polynucleotides.

### SUMMARY OF THE INVENTION

This invention relates to novel human polynucleotides and variants thereof, their encoded polypeptides and variants thereof, to genes corresponding to these polynucleotides and to proteins expressed by the genes. The invention also relates to diagnostics and therapeutics comprising such novel human polynucleotides, their corresponding genes or gene products, including probes, antisense nucleotides, and antibodies. The polynucleotides of the invention correspond to a polynucleotide comprising the sequence information of at least one of SEQ ID NOS:1-2396.

Various aspects and embodiments of the invention will be readily apparent to the ordinarily skilled artisan upon reading the description provided herein.

### DETAILED DESCRIPTION OF THE INVENTION

The invention relates to polynucleotides comprising the disclosed nucleotide sequences, to full length cDNA, mRNA genomic sequences, and genes corresponding to these sequences and degenerate variants thereof, and to polypeptides encoded by the polynucleotides of the invention and polypeptide variants. The following detailed description describes the polynucleotide compositions encompassed by the invention, methods for obtaining cDNA or genomic DNA encoding a full-length gene product, expression of these polynucleotides and genes, identification of structural motifs of the polynucleotides and genes, identification of the function of a gene product encoded by a gene corresponding to a polynucleotide of the invention, use of the provided polynucleotides as probes and in mapping and in tissue profiling, use of the corresponding polypeptides and other gene products to raise antibodies, and use of the polynucleotides and their encoded gene products for therapeutic and diagnostic purposes.

Polynucleotide Compositions

The scope of the invention with respect to polynucleotide compositions includes, but is not necessarily limited to, polynucleotides having a sequence set forth in any one of SEQ ID NOS: -2396; polynucleotides obtained from the biological materials described herein or other biological sources (particularly human sources) by hybridization under stringent conditions (particularly conditions of high stringency); genes corresponding to the provided polynucleotides; variants of the provided polynucleotides and their corresponding genes, particularly those variants that retain a biological activity of the encoded gene product (e.g., a biological activity ascribed to a gene product corresponding to the provided polynucleotides as a result of the assignment of the gene product to a protein family(ies) and/or identification of a functional domain present in the gene product). Other nucleic acid compositions contemplated by and within the scope of the present invention will be readily apparent to one of ordinary skill in the art when provided with the disclosure here. “Polynucleotide” and “nucleic acid” as used herein with reference to nucleic acids of the composition is not intended to be limiting as to the length or structure of the nucleic acid unless specifically indicted.

The invention features polynucleotides that are expressed in human tissue, specifically human colon, breast, and/or lung tissue. Novel nucleic acid compositions of the invention of particular interest comprise a sequence set forth in any one of SEQ ID NOS:1-2396 or an identifying -sequence thereof. An “identifying sequence” is a contiguous sequence of residues at least about 10 nt to about 20 nt in length, usually at least about 50 nt to about 100 nt in length, that uniquely identifies a polynucleotide sequence, e.g., exhibits less than 90%, usually less than about 80% to about 85% sequence identity to any contiguous nucleotide sequence of more than about 20 nt. Thus, the subject novel nucleic acid compositions include full length cDNAs or mRNAs that encompass an identifying sequence of contiguous nucleotides from any one of SEQ ID NOS:1-2396.

The polynucleotides of the invention also include polynucleotides having sequence similarity or sequence identity. Nucleic acids having sequence similarity are detected by hybridization under low stringency conditions, for example, at 50° C. and 10× SSC (0.9 M saline/0.09 M sodium citrate) and remain bound when subjected to washing at 55° C. in 1× SSC. Sequence identity can be determined by hybridization under stringent conditions, for example, at 50° C. or higher and 0.1× SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences (SEQ ID NOS:1-2396) under stringent hybridization conditions. By using probes, particularly labeled probes of DNA sequences, one can isolate homologous or related genes. The source of homologous genes can be any species, e.g. primate species, particularly human; rodents, such as rats and mice; canines, felines, bovines, ovines, equines, yeast, nematodes, etc.

Preferably, hybridization is performed using at least 15 contiguous nucleotides (nt) of at least one of SEQ ID NOS:1-2396. That is, when at least 15 contiguous nt of one of the disclosed SEQ ID NOS. is used as a probe, the probe will preferentially hybridize with a nucleic acid comprising the complementary sequence, allowing the identification and retrieval of the nucleic acids that uniquely hybridize to the selected probe. Probes from more than one SEQ ID NO. can hybridize with the same nucleic acid if the cDNA from which they were derived corresponds to one mRNA. Probes of more than 15 nt can be used, e.g., probes of from about 18 nt to about 100 nt, but -15 nt represents sufficient sequence for unique identification.

The polynucleotides of the invention also include naturally occurring variants of the nucleotide sequences (e.g., degenerate variants, allelic variants, etc.). Variants of the polynucleotides of the invention are identified by hybridization of putative variants with nucleotide sequences disclosed herein, preferably by hybridization under stringent conditions. For example, by using appropriate wash conditions, variants of the polynucleotides of the invention can be identified where the allelic variant exhibits at most about 25-30% base pair (bp) mismatches relative to the selected polynucleotide probe. In general, allelic variants contain 15-25% bp mismatches, and can contain as little as even 5-15%, or 2-5%, or 1-2% bp mismatches, as well as a single bp mismatch.

The invention also encompasses homologs corresponding to the polynucleotides of SEQ ID NOS:1-2396, where the source of homologous genes can be any mammalian species, e.g. primate species, particularly human; rodents, such as rats; canines, felines, bovines, ovines, equines, yeast, nematodes, etc. Between mammalian species, e.g., human and mouse, homologs generally have substantial sequence similarity, e.g., at least 75% sequence identity, usually at least 90%, more usually at least 95% between nucleotide sequences. Sequence similarity is calculated based on a reference sequence, which may be a subset of a larger sequence, such as a conserved motif, coding region, flanking region, etc. A reference sequence will usually be at least about 18 contiguous nt long, more usually at least about 30 nt long, and may extend to the complete sequence that is being compared. Algorithms for sequence analysis are known in the art, such as gapped BLAST, described in Altschul, et al. Nucleic Acids Res. (1997) 25:3389-3402.

In general, variants of the invention have a sequence identity greater than at least about 65%, preferably at least about 75%, more preferably at least about 85%, and can be greater than at least about 90% or more as determined by the Smith-Waterman homology search algorithm as implemented in MPSRCH program (Oxford Molecular). For the purposes of this invention, a preferred method of calculating percent identity is the Smith-Waterman algorithm, using the following. Global DNA sequence identity must be greater than 65% as determined by the Smith-Waterman homology search algorithm as implemented in MPSRCH program (Oxford Molecular) using an affine gap search with the following search parameters: gap open penalty, 12; and gap extension penalty, 1.

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.). The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kbp or smaller; and substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

The nucleic acid compositions of the subject invention can encode all or a part of the subject polypeptides. Double or single stranded fragments can be obtained from the DNA sequence by chemically synthesizing oligonucleotides in accordance with conventional methods, by restriction enzyme digestion, by PCR amplification, etc. Isolated polynucleotides and polynucleotide fragments of the invention comprise at least about 10, about 15, about 20, about 35, about 50, about 100, about 150 to about 200, about 250 to about 300, or about 350 contiguous nt selected from the polynucleotide sequences as shown in SEQ ID NOS:1-2396. For the most part, fragments will be of at least 15 nt, usually at least 18 nt or 25 nt, and up to at least about 50 contiguous nt in length or more. In a preferred embodiment, the polynucleotide molecules comprise a contiguous sequence of at least 12 nt selected from the group consisting of the polynucleotides shown in SEQ ID NOS:1-2396.

Probes specific to the polynucleotides of the invention can be generated using the polynucleotide sequences disclosed in SEQ ID NOS:1-2396. The probes are preferably at least about a 12, 15, 16, 18, 20, 22, 24, or 25 nt fragment of a corresponding contiguous sequence of SEQ ID NOS:1-2396, and can be less than 2, 1, 0.5, 0.1, or 0.05 kb in length. The probes can be synthesized chemically or can be generated from longer polynucleotides using restriction enzymes. The probes can be labeled, for example, with a radioactive, biotinylated, or fluorescent tag. Preferably, probes are designed based upon an identifyng sequence of a polynucleotide of one of SEQ ID NOS:1-2396. More preferably, probes are designed based on a contiguous sequence of one of the subject polynucleotides that remain unmasked following application of a masking program for masking low complexity (e.g., XBLAST) to the sequence., ie., one would select an unmasked region, as indicated by the polynucleotides outside the poly-n stretches of the masked sequence produced by the masking program.

The polynucleotides of the subject invention are isolated and obtained in substantial purity, generally as other than an intact chromosome. Usually, the polynucleotides, either as DNA or RNA, will be obtained substantially free of other naturally-occurring nucleic acid sequences, generally being at least about 50%, usually at least about 90% pure and are typically “recombinant”, e.g., flanked by one or more nucleotides with which it is not normally associated on a naturally occurring chromosome.

The polynucleotides of the invention can be provided as a linear molecule or within a circular molecule, and can be provided within autonomously replicating molecules (vectors) or -within molecules without replication sequences. Expression of the polynucleotides can be regulated by their own or by other regulatory sequences known in the art. The polynucleotides of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

The subject nucleic acid compositions can be used to, for example, produce polypeptides, as probes for the detection of mRNA of the invention in biological samples (e.g., extracts of human cells) to generate additional copies of the polynucleotides, to generate ribozymes or antisense oligonucleotides, and as single stranded DNA probes or as triple-strand forming oligonucleotides. The probes described herein can be used to, for example, determine the presence or absence of the polynucleotide sequences as shown in SEQ ID NOS:1-2396 or variants thereof in a sample. These and other uses are described in more detail below.

Use of Polynucleotides to Obtain Full-Length cDNA, Gene, and Promoter Region

Full-length cDNA molecules comprising the disclosed polynucleotides are obtained as follows. A polynucleotide having a sequence of one of SEQ ID NOS:1-2396, or a portion thereof comprising at least 12, 15, 18, or 20 nt, is used as a hybridization probe to detect hybridizing members of a cDNA library using probe design methods, cloning methods, and clone selection techniques such as those described in U.S. Pat. No. 5,654,173. Libraries of cDNA are made from selected tissues, such as normal or tumor tissue, or from tissues of a mammal treated with, for example, a pharmaceutical agent. Preferably, the tissue is the same as the tissue from which the polynucleotides of the invention were isolated, as both the polynucleotides described herein and the cDNA represent expressed genes. Most preferably, the cDNA library is made from the biological material described herein in the Examples. The choice of cell type for library construction can be made after the identity of the protein encoded by the gene corresponding to the polynucleotide of the invention is known. This will indicate which tissue and cell types are likely to express the related gene, and thus represent a suitable source for the mRNA for generating the cDNA. Where the provided polynucleotides are isolated from cDNA libraries, the libraries are prepared from mRNA of human colon cells, more preferably, human colon cancer cells, even more preferably, from a highly metastatic colon cell, Km12L4.

Techniques for producing and probing nucleic acid sequence libraries are described, for example, in Sambrook et al., Molecular Cloning: A Laboratory Manual, 2nd Ed., (1989) Cold Spring Harbor Press, Cold Spring Harbor, N.Y. The cDNA can be prepared by using primers based on sequence from SEQ ID NOS:1-2396. hi one embodiment, the cDNA library can be made from only poly-adenylated mRNA. Thus, poly-T primers can be used to prepare cDNA from the mRNA.

Members of the library that are larger than the provided polynucleotides, and preferably that encompass the complete coding sequence of the native message, are obtained. In order to confirm that the entire cDNA has been obtained, RNA protection experiments are performed as follows. Hybridization of a full-length cDNA to an mRNA will protect the RNA from RNase degradation. If the cDNA is not full length, then the portions of the mRNA that are not hybridized will be subject to RNase degradation. This is assayed, as is known in the art, by changes in electrophoretic mobility on polyacrylamide gels, or by detection of released monoribonucleotides. Sambrook et al., Molecular Cloning: A Laboratory Manual, 2nd Ed., (1989) Cold Spring Harbor Press, Cold Spring Harbor, N.Y. In order to obtain additional sequences 5'to the end of a partial cDNA, 5′ RACE (PCR Protocols: A Guide to Methods and Applications, (1990) Academic Press, Inc.) can be performed.

Genomic DNA is isolated using the provided polynucleotides in a manner similar to the isolation of full-length cDNAs. Briefly, the provided polynucleotides, or portions thereof, are used as probes to libraries of genomic DNA. Preferably, the library is obtained from the cell type that was used to generate the polynucleotides of the invention, but this is not essential. Most preferably, the genomic DNA is obtained from the biological material described herein in the Examples. Such libraries can be in vectors suitable for carrying large segments of a genome, such as P1 or YAC, as described in detail in Sambrook et al., 9.4-9.30. Lin addition, genomic sequences can be isolated from human BAC libraries, which are commercially available from Research Genetics, Inc., Huntsville, Ala., USA, for example. hi order to obtain additional 5′ or 3′ sequences, chromosome walking is performed, as described in Sambrook et al., such that adjacent and overlapping fragments of genomic DNA are isolated. These are mapped and pieced together, as is known in the art, using restriction digestion enzymes and DNA ligase.

Using the polynucleotide sequences of the invention, corresponding full-length genes can be isolated using both classical and PCR methods to construct and probe cDNA libraries. Using either method, Northern blots, preferably, are performed on a number of cell types to determine which cell lines express the gene of interest at the highest level. Classical methods of constructing cDNA libraries are taught in Sambrook et al., supra. With these methods, cDNA can be produced from -mRNA and inserted into viral or expression vectors. Typically, libraries of mRNA comprising poly(A) tails can be produced with poly(T) primers. Similarly, cDNA libraries can be produced using the instant sequences as primers.

PCR methods are used to amplify the members of a cDNA library that comprise the desired insert. In this case, the desired insert will contain sequence from the full length cDNA that corresponds to the instant polynucleotides. Such PCR methods include gene trapping and RACE methods. Gene trapping entails inserting a member of a cDNA library into a vector. The vector then is denatured to produce single stranded molecules. Next, a substrate-bound probe, such a biotinylated oligo, is used to trap cDNA inserts of interest. Biotinylated probes can be linked to an avidin-bound solid substrate. PCR methods can be used to amplify the trapped cDNA. To trap sequences corresponding to the full length genes, the labeled probe sequence is based on the polynucleotide sequences of the invention. Random primers or primers specific to the library vector can be used to amplify the trapped cDNA. Such gene trapping techniques are described in Gruber et al., WO 95/04745 and Gruber et al., U.S. Pat. No. 5,500,356. Kits are commercially available to perform gene trapping experiments from, for example, Life Technologies, Gaithersburg, Md., USA.

“Rapid amplification of cDNA ends,” or RACE, is a PCR method of amplifying cDNAs from a number of different RNAs. The cDNAs are ligated to an oligonucleotide linker, and amplified by PCR using two primers. One primer is based on sequence from the instant polynucleotides, for which full length sequence is desired, and a second primer comprises sequence that hybridizes to the oligonucleotide linker to amplify the cDNA. A description of this methods is reported in WO 97/19110. In preferred embodiments of RACE, a common primer is designed to anneal to an arbitrary adaptor sequence ligated to cDNA ends (Apte and Siebert, Biotechniques (1993) 15:890-893; Edwards et al., Nuc. Acids Res. (1991) 19:5227-5232). When a single gene-specific RACE primer is paired with the common primer, preferential amplification of sequences between the single gene specific primer and the common primer occurs. Commercial cDNA pools modified for use in RACE are available.

Another PCR-based method generates full-length cDNA library with anchored ends without needing specific knowledge of the cDNA sequence. The method uses lock-docking primers (I-VI), where one primer, poly TV (I-III) locks over the polyA tail of eukaryotic mRNA producing first strand synthesis and a second primer, polyGH (IV-VI) locks onto the polyC tail added by terminal deoxynucleotidyl transferase (TdT)(see, e.g., WO 96/40998).

The promoter region of a gene generally is located 5′ to the initiation site for RNA polymerase II. Hundreds of promoter regions contain the “TATA” box, a sequence such as TATTA or TATAA, which is sensitive to mutations. The promoter region can be obtained by performing 5′RACE using a primer from the coding region of the gene. Alternatively, the cDNA can be used as a probe for the genomic sequence, and the region 5′ to the coding region is identified by “walking up.” If the gene is highly expressed or differentially expressed, the promoter from the gene can be of use in a regulatory construct for a heterologous gene.

Once the full-length cDNA or gene is obtained, DNA encoding variants can be prepared by site-directed mutagenesis, described in detail in Sambrook et al., 15.3-15.63. The choice of codon or nucleotide to be replaced can be based on disclosure herein on optional changes in amino acids to achieve altered protein structure and/or function.

As an alternative method to obtaining DNA or RNA from a biological material, nucleic acid comprising nucleotides having the sequence of one or more polynucleotides of the invention can be synthesized. Thus, the invention encompasses nucleic acid molecules ranging in length from 15 nt (corresponding to at least 15 contiguous nt of one of SEQ ID NOS:1-2396) up to a maximum length suitable for one or more biological manipulations, including replication and expression, of the nucleic acid molecule. The invention includes but is not limited to (a) nucleic acid having the size of a full gene, and comprising at least one of SEQ ID NOS:1-2396; (b) the nucleic acid of (a) also comprising at least one additional gene, operably linked to permit expression of a fusion protein; (c) an expression vector comprising (a) or (b); (d) a plasmid comprising (a) or (b); and (e) a recombinant viral particle comprising (a) or (b). Once provided with the polynucleotides disclosed herein, construction or preparation of (a)- (e) are well within the skill in the art.

The sequence of a nucleic acid comprising at least 15 contiguous nt of at least any one of SEQ ID NOS:1-2396, preferably the entire sequence of at least any one of SEQ ID NOS:1-2396, is not limited and can be any sequence of A, T, G, and/or C (for DNA) and A, U, G, and/or C (for RNA) or modified bases thereof, including inosine and pseudouridine. The choice of sequence will depend on the desired function and can be dictated by coding regions desired, the intron-like regions desired, and the regulatory regions desired. Where the entire sequence of any one of SEQ ID NOS:1-2396 is within the nucleic acid, the nucleic acid obtained is referred to herein as a polynucleotide comprising the sequence of any one of SEQ ID NOS:1-2396.

Expression of Polypeptide Encoded by Full-Length cDNA or Full-Length Gene

The provided polynucleotides (e.g., a polynucleotide having a sequence of one of SEQ ID NOS:1-2396), the corresponding cDNA, or the full-length gene is used to express a partial or complete gene product. Constructs of polynucleotides having sequences of SEQ ID NOS:1-2396 can also be generated synthetically. Alternatively, single-step assembly of a gene and entire plasmid from large numbers of oligodeoxyribonucleotides is described by, e.g. Stemmer et al., Gene (Amsterdam) (1995) 164(1):49-53. In this method, assembly PCR (the synthesis of long DNA sequences from large numbers of oligodeoxyribonucleotides (oligos)) is described. The method is derived from DNA shuffling (Stemmer, Nature (1994) 370:389-391), and does not rely on DNA ligase, but instead relies on DNA polymerase to build increasingly longer DNA fragments during the assembly process.

Appropriate polynucleotide constructs are purified using standard recombinant DNA techniques as described in, for example, Sambrook et al., Molecular Cloning: A Laboratory Manual, 2nd Ed., (1989) Cold Spring Harbor Press, Cold Spring Harbor, N.Y, and under current regulations described in United States Dept. of HHS, National Institute of Health (NIH) Guidelines for Recombinant DNA Research. The gene product encoded by a polynucleotide of the invention is expressed in any expression system, including, for example, bacterial, yeast, insect, amphibian and mammalian systems. Vectors, host cells and methods for obtaining expression in same are well known in the art. Suitable vectors and host cells are described in U.S. Pat. No. 5,654,173.

Polynucleotide molecules comprising a polynucleotide sequence provided herein are generally propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. Methods for preparation of vectors comprising a desired sequence are well known in the art.

The polynucleotides set forth in SEQ ID NOS:1-2396 or their corresponding full-length polynucleotides are linked to regulatory sequences as appropriate to obtain the desired expression properties. These can include promoters (attached either at the 5′ end of the sense strand or at the 3′ end of the antisense strand), enhancers, terminators, operators, repressors, and inducers. The promoters can be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art can be used.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Once the gene corresponding to a selected polynucleotide is identified, its expression can be regulated in the cell to which the gene is native. For example, an endogenous gene of a cell can be regulated by an exogenous regulatory sequence as disclosed in U.S. Pat. No. 5,641,670.

Identification of Functional and Structural Motifs of Novel Genes Screening Against Publicly Available Databases

Translations of the nucleotide sequence of the provided polynucleotides, cDNAs or full genes can be aligned with individual known sequences. Similarity with individual sequences can be used to determine the activity of the polypeptides encoded by the polynucleotides of the invention. Also, sequences exhibiting similarity with more than one individual sequence can exhibit activities that are characteristic of either or both individual sequences.

The full length sequences and fragments of the polynucleotide sequences of the nearest neighbors can be used as probes and primers to identify and isolate the full length sequence corresponding to provided polynucleotides. The nearest neighbors can indicate a tissue or cell type to be used to construct a library for the full-length sequences corresponding to the provided polynucleotides.

Typically, a selected polynucleotide is translated in all six frames to determine the best alignment with the individual sequences. The sequences disclosed herein in the Sequence Listing are in a 5′ to 3′ orientation and translation in three frames can be sufficient (with a few specific exceptions as described in the Examples). These amino acid sequences are referred to, generally, as query sequences, which will be aligned with the individual sequences. Databases with individual sequences are described in “Computer Methods for Macromolecular Sequence Analysis” Methods in Enzymology (1996) 266, Doolittle, Academic Press, Inc., a division of Harcourt Brace & Co., San Diego, Calif., USA. Databases include GenBank, EMBL, and DNA Database of Japan (DDBJ).

Query and individual sequences can be aligned using the methods and computer programs described above, and include BLAST 2.0 ((National Center for Biotechnology Information, Bethesda, Md.). See also Altschul, et al. Nucleic Acids Res. (1997) 25:3389-3402. Another alignment algorithm is Fasta, available in the Genetics Computing Group (GCG) package, Madison, Wisconsin, USA, a wholly owned subsidiary of Oxford Molecular Group, Inc. Other techniques for alignment are described in Doolittle, supra. Preferably, an alignment program that permits gaps in the sequence is utilized to align the sequences. The Smith-Waterman is one type of algorithm that permits gaps in sequence alignments. See Meth. Mol. Biol. (1997) 70: 173-187. Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to identify sequences that are distantly related matches, and is especially tolerant of small gaps and nucleotide sequence errors. Amino acid sequences encoded by the provided polynucleotides can be used to search both protein and DNA databases. Incorporated 2(1- herein by reference are all sequences that have been made public as of the filing date of this application by any of the DNA or protein sequence databases, including the patent databases (e.g., GeneSeq). Also incorporated by reference are those sequences that have been submitted to these databases as of the filing date of the present application but not made public until after the filing date of the present application.

Results of individual and query sequence alignments can be divided into three categories: high similarity, weak similarity, and no similarity. Individual alignment results ranging from high similarity to weak similarity provide a basis for determining polypeptide activity and/or structure. Parameters for categorizing individual results include: percentage of the alignment region length where the strongest alignment is found, percent sequence identity, and p value. The percentage of the alignment region length is calculated by counting the number of residues of the individual sequence found in the region of strongest alignment, e.g., contiguous region of the individual sequence that contains the greatest number of residues that are identical to the residues of the corresponding region of the aligned query sequence. This number is divided by the total residue length of the query sequence to calculate a percentage. For example, a query sequence of 20 amino acid residues might be aligned with a 20 amino acid region of an individual sequence. The individual sequence might be identical to amino acid residues 5, 9-15, and 17-19 of the query sequence. The region of strongest alignment is thus the region stretching from residue 9-19, an 11 amino acid stretch. The percentage of the alignment region length is: 11 (length of the region of strongest alignment) divided by (query sequence length) 20 or 55%.

Percent sequence identity is calculated by counting the number of amino acid matches between the query and individual sequence and dividing total number of matches by the number of residues of the individual sequences found in the region of strongest alignment. Thus, the percent identity in the example above would be 10 matches divided by 11 amino acids, or approximately, 90.9%

P value is the probability that the alignment was produced by chance. For a single alignment, the p value can be calculated according to Karlin et al., Proc. Natl. Acad. Sci. (1990) 87:2264 and Karlin et al., Proc. Natl. Acad. Sci. (1993) 90. The p value of multiple alignments using the same query sequence can be calculated using an heuristic approach described in Altschul et al., Nat. Genet. (1994) 6:119. Alignment programs such as BLAST program can calculate the p value. See also Altschul et al., Nucleic Acids Res. (1997) 25:3389-3402.

Another factor to consider for determining identity or similarity is the location of the similarity or identity. Strong local alignment can indicate similarity even if the length of alignment is short. Sequence identity scattered throughout the length of the query sequence also can indicate a similarity between the query and profile sequences. The boundaries of the region where the sequences align can be determined according to Doolittle, supra; BLAST 2.0 (see, e.g., Altschul, et al. Nucleic Acids Res. (1997) 25:3389-3402) or FAST programs; or by determining the area where sequence identity is highest.

High Similarity.

In general, in alignment results considered to be of high similarity, the percent of the alignment region length is typically at least about 55% of total length query sequence; more typically, at least about 58%; even more typically; at least about 60% of the total residue length of the query sequence. Usually, percent length of the alignment region can be as much as about 62%; more usually, as much as about 64%; even more usually, as much as about 66%. Further, for high similarity, the region of alignment, typically, exhibits at least about 75% of sequence identity; more typically, at least about 78%; even more typically; at least about 80% sequence identity. Usually, percent sequence identity can be as much as about 82%; more usually, as much as about 84%; even more usually, as much as about 86%.

The p value is used in conjunction with these methods. If high similarity is found, the query sequence is considered to have high similarity with a profile sequence when the p value is less than or equal to about 10⁻²; more usually; less than or equal to about 10⁻³; even more usually; less than or equal to about 10⁻⁴. More typically, the p value is no more than about 10⁻⁵; more typically; no more than or equal to about 10⁻¹⁰; even more typically; no more than or equal to about 10⁻¹⁵ for the query sequence to be considered high similarity.

Weak Similarity.

In general, where alignment results considered to be of weak similarity, there is no minimum percent length of the alignment region nor minimum length of alignment. A better showing of weak similarity is considered when the region of alignment is, typically, at least about 15 amino acid residues in length; more typically, at least about 20; even more typically; at least about 25 amino acid residues in length. Usually, length of the alignment region can be as much as about 30 amino acid residues; more usually, as much as about 40; even more usually, as much as about 60 amino acid residues. Further, for weak similarity, the region of alignment, typically, exhibits at least about 35% of sequence identity; more typically, at least about 40%; even more typically; at least about 45% sequence identity. Usually, percent sequence identity can be as much as about 50%; more usually, as much as about 55%; even more usually, as much as about 60%.

If low similarity is found, the query sequence is considered to have weak similarity with a profile sequence when the p value is usually less than or equal to about 10⁻²; more usually; less than or equal to about 10⁻³; even more usually; less than or equal to about 10⁻⁴. More typically, the p value is no more than about 10⁻⁵; more usually; no more than or equal to about 10⁻¹⁰; even more usually; no more than or equal to about 10⁻¹⁵ for the query sequence to be considered weak similarity.

Similarity Determined by Sequence Identity Alone.

Sequence identity alone can be used to determine similarity of a query sequence to an individual sequence and can indicate the activity of the sequence. Such an alignment, preferably, permits gaps to align sequences. Typically, the query sequence is related to the profile sequence if the sequence identity over the entire query sequence is at least about 15%; more typically, at least about 20%; even more typically, at least about 25%; even more typically, at least about 50%. Sequence identity alone as a measure of similarity is most useful when the query sequence is usually, at least 80 residues in length; more usually, 90 residues; even more usually, at least 95 amino acid residues in length. More typically, similarity can be concluded based on sequence identity alone when the query sequence is preferably 100 residues in length; more preferably, 120 residues in length; even more preferably, 150 amino acid residues in length.

Alignments with Profile and Multiple Aligned Sequences.

Translations of the provided polynucleotides can be aligned with amino acid profiles that define either protein families or common motifs. Also, translations of the provided polynucleotides can be aligned to multiple sequence alignments (MSA) comprising the polypeptide sequences of members of protein families or motifs. Similarity or identity with profile sequences or MSAs can be used to determine the activity of the gene products (e.g., polypeptides) encoded by the provided polynucleotides or corresponding cDNA or genes. For example, sequences that show an identity or similarity with a chemokine profile or MSA can exhibit chemokine activities.

Profiles can designed manually by (1) creating an MSA, which is an alignment of the amino acid sequence of members that belong to the family and (2) constructing a statistical representation of the alignment. Such methods are described, for example, in Bimey et al., Nucl. Acid Res. (1996) 24(14): 2730-2739. MSAs of some protein families and motifs are publicly available. For example, the Pfam database available from Washington University (St. Louis, Mo.) includes MSAs of 547 different families and motifs. These MSAs are described also in Sonnhammer et al., Proteins (1997) 28: 405-420. Other publicaly available sources include those over the world wide web provided by the European Molecular Biology Laboratory (Heidelberg, Germany). A brief description of these MSAs is reported in Pascarella et al., Prot. Eng. (1996) 9(3):249-251. Techniques for building profiles from MSAs are described in Sonnhammer et al., supra; Birney et al., supra; and “Computer Methods for Macromolecular Sequence Analysis,” Methods in Enzymology (1996) 266, Doolittle, Academic Press, Inc., San Diego, Calf., USA.

Similarity between a query sequence and a protein family or motif can be determined by (a) comparing the query sequence against the profile and/or (b) aligning the query sequence with the members of the family or motif. Typically, a program such as Searchwise is used to compare the query sequence to the statistical representation of the multiple alignment, also known as a profile (see Bimey et al., supra). Other techniques to compare the sequence and profile are described in Sonnhammer et al., supra and Doolittle, supra.

Next, methods described by Feng et al., J. Mol. Evol. (1987) 25:351 and Higgins et al., CABIOS (1989) 5:151 can be used align the query sequence with the members of a family or motif, also known as a MSA. Sequence alignments can be generated using any of a variety of software tools. Examples include PileUp, which creates a multiple sequence alignment, and is described in Feng et al., J. Mol. Evol. (1987) 25:351. Another method, GAP, uses the alignment method of Needleman et al., J. Mol. Biol. (1970) 48:443. GAP is best suited for global alignment of sequences. A third method, BestFit, functions by inserting gaps to maximize the number of matches using the local homology algorithm of Smith et al., Adv. Appl. Math. (1981) 2:482. In general, the following factors are used to determine if a similarity between a query sequence and a profile or MSA exists: (1) number of conserved residues found in the query sequence, (2) percentage of conserved residues found in the query sequence, (3) number of frameshifts, and (4) spacing between conserved residues.

Some alignment programs that both translate and align sequences can make any number of frameshifts when translating the nucleotide sequence to produce the best alignment. The fewer frameshifts needed to produce an alignment, the stronger the similarity or identity between the query and profile or MSAs. For example, a weak similarity resulting from no frameshifts can be a better indication of activity or structure of a query sequence, than a strong similarity resulting from two frameshifts. Preferably, three or fewer frameshifts are found in an alignment; more preferably two or fewer frameshifts; even more preferably, one or fewer frameshifts; even more preferably, no frameshifts are found in an alignment of query and profile or MSAs.

Conserved residues are those amino acids found at a particular position in all or some of the family or motif members. Alternatively, a position is considered conserved if only a certain class of amino acids is found in a particular position in all or some of the family members. For example, the N-terminal position can contain a positively charged amino acid, such as lysine, arginine, or histidine.

Typically, a residue of a polypeptide is conserved when a class of amino acids or a single amino acid is found at a particular position in at least about 40% of all class members; more typically, at least about 50%; even more typically, at least about 60% of the members. Usually, a residue is conserved when a class or single amino acid is found in at least about 70% of the members of a family or motif, more usually, at least about 80%; even more usually, at least about 90%; even more usually, at least about 95%.

A residue is considered conserved when three unrelated amino acids are found at a particular position in the some or all of the members; more usually, two unrelated amino acids. These residues are conserved when the unrelated amino acids are found at particular positions in at least about 40% of all class member; more typically, at least about 50%; even more typically, at least about 60% of the members. Usually, a residue is conserved when a class or single amino acid is found in at least about 70% of the members of a family or motif, more usually, at least about 80%; even more usually, at least about 90%; even more usually, at least about 95%.

A query sequence has similarity to a profile or MSA when the query sequence comprises at least about 25% of the conserved residues of the profile or MSA; more usually, at least about 30%; even more usually; at least about 40%. Typically, the query sequence has a stronger similarity to a profile sequence or MSA when the query sequence comprises at least about 45% of the conserved residues of the profile or MSA; more typically, at least about 50%; even more typically; at least about 55%.

Identification of Secreted & Membrane-Bound Polypeptides

Both secreted and membrane-bound polypeptides of the present invention are of particular interest. For example, levels of secreted polypeptides can be assayed in body fluids that are convenient, such as blood, plasma, serum, and other body fluids such as urine, prostatic fluid and semen. Membrane-bound polypeptides are useful for constructing vaccine antigens or inducing an immune response. Such antigens would comprise all or part of the extracellular region of the membrane-bound polypeptides. Because both secreted and membrane-bound polypeptides comprise a fragment of contiguous hydrophobic amino acids, hydrophobicity predicting algorithms can be used to identify such polypeptides.

A signal sequence is usually encoded by both secreted and membrane-bound polypeptide genes to direct a polypeptide to the surface of the cell. The signal sequence usually comprises a stretch of hydrophobic residues. Such signal sequences can fold into helical structures. Membrane-bound polypeptides typically comprise at least one transmembrane region that possesses a stretch of hydrophobic amino acids that can transverse the membrane. Some transmembrane regions also exhibit a helical structure. Hydrophobic fragments within a polypeptide can be identified by using computer algorithms. Such algorithms include Hopp & Woods. Proc. Natl. Acad. Sci. USA (1981) 78:3824-3828; Kyte & Doolittle, J. Mol. Biol. (1982) 157: 105-132; and RAOAR algorithm, Degli Esposti et al., Eur. J. Biochem. (1990) 190: 207-219.

Another method of identifying secreted and membrane-bound polypeptides is to translate the polynucleotides of the invention in all six frames and determine if at least 8 contiguous hydrophobic amino acids are present. Those translated polypeptides with at least 8; more typically, 10; even more typically, 12 contiguous hydrophobic amino acids are considered to be either a putative secreted or membrane bound polypeptide. Hydrophobic amino acids include alanine, glycine, histidine, isoleucine, leucine, lysine, methionine, phenylalanine, proline, threonine, tryptophan, tyrosine, and valine.

Identification of the Function of an Expression Product of a Full-Length Gene

Ribozymes, antisense constructs, and dominant negative mutants can be used to determine function of the expression product of a gene corresponding to a polynucleotide provided herein. These methods and compositions are particularly useful where the provided novel polynucleotide exhibits no significant or substantial homology to a sequence encoding a gene of known function. Antisense molecules and ribozymes can be constructed from synthetic polynucleotides. Typically, the phosphoramidite method of oligonucleotide synthesis is used. See Beaucage et al., Tet. Lett. (1981) 22:1859 and U.S. Pat. No. 4,668,777. Automated devices for synthesis are available to create oligonucleotides using this chemistry. Examples of such devices include Biosearch 8600, Models 392 and 394 by Applied Biosystems, a division of Perkin-Elmer Corp., Foster City, Calif., USA; and Expedite by Perceptive Biosystems, Framingham, Mass., USA. Synthetic RNA, phosphate analog oligonucleotides, and chemically derivatized oligonucleotides can also be produced, and can be covalently attached to other molecules. RNA oligonucleotides can be synthesized, for example, using RNA phosphoramidites. This method can be performed on an automated synthesizer, such as Applied Biosystems, Models 392 and 394, Foster City, Calif., USA.

Phosphorothioate oligonucleotides can also be synthesized for antisense construction. A sulfurizing reagent, such as tetraethylthiruam disulfide (TETD) in acetonitrile can be used to convert the internucleotide cyanoethyl phosphite to the phosphorothioate triester within 15 minutes at room temperature. TETD replaces the iodine reagent, while all other reagents used for standard phosphoramidite chemistry remain the same. Such a synthesis method can be automated using Models 392 and 394 by Applied Biosystems, for example.

Oligonucleotides of up to 200 nt can be synthesized, more typically, 100 nt, more typically 50 nt; even more typically 30 to 40 nt. These synthetic fragments can be annealed and ligated together to construct larger fragments. See, for example, Sambrook et al., supra. Trans-cleaving catalytic RNAs (ribozymes) are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting the phenotypic effect. One commonly used ribozyme motif is the hammerhead, for which the substrate sequence requirements are minimal. Design of the hammerhead ribozyme, as well as therapeutic uses of ribozymes, are disclosed in Usman et al., Current Opin. Struct. Biol. (1996) 6:527. Methods for production of ribozymes, including hairpin structure ribozyme fragments, methods of increasing ribozyme specificity, and the like are known in the art.

The hybridizing region of the ribozyme can be modified or can be prepared as a branched structure as described in Horn and Urdea, Nucleic Acids Res. (1989) 17:6959. The basic structure of the ribozymes can also be chemically altered in ways familiar to those skilled in the art, and chemically synthesized ribozymes can be administered as synthetic oligonucleotide derivatives modified by monomeric units. In a therapeutic context, liposome mediated delivery of ribozymes improves cellular uptake, as described in Birikh et al., Eur. J. Biochem. (1997) 245:1.

Antisense nucleic acids are designed to specifically bind to RNA, resulting in the formation of RNA-DNA or RNA-RNA hybrids, with an arrest of DNA replication, reverse transcription or messenger RNA translation. Antisense polynucleotides based on a selected polynucleotide sequence can interfere with expression of the corresponding gene. Antisense polynucleotides are typically generated within the cell by expression from antisense constructs that contain the antisense strand as the transcribed strand. Antisense polynucleotides based on the disclosed polynucleotides will bind and/or interfere with the translation of mRNA comprising a sequence complementary to the antisense polynucleotide. The expression products of control cells and cells treated with the antisense construct are compared to detect the protein product of the gene corresponding to the polynucleotide upon which the antisense construct is based. The protein is isolated and identified using routine biochemical methods.

Given the extensive background literature and clinical experience in antisense therapy, one skilled in the art can use selected polynucleotides of the invention as additional potential therapeutics. The choice of polynucleotide can be narrowed by first testing them for binding to “hot spot” regions of the genome of cancerous cells. If a polynucleotide is identified as binding to a “hot spot”, testing the polynucleotide as an antisense compound in the corresponding cancer cells is warranted.

As an alternative method for identifying function of the gene corresponding to a polynucleotide disclosed herein, dominant negative mutations are readily generated for corresponding proteins that are active as homomultimers. A mutant polypeptide will interact with wild-type polypeptides (made from the other allele) and form a non-functional multimer. Thus, a mutation is in a substrate-binding domain, a catalytic domain, or a cellular localization domain. Preferably, the mutant polypeptide will be overproduced. Point mutations are made that have such an effect. In addition, fusion of different polypeptides of various lengths to the terminus of a protein can yield dominant negative mutants. General strategies are available for making dominant negative mutants (see, e.g., Herskowitz, Nature (1987) 329:219). Such techniques can be used to create loss of function mutations, which are useful for determining protein function.

Polypeptides and Variants Thereof

The polypeptides of the invention include those encoded by the disclosed polynucleotides, as well as nucleic acids that, by virtue of the degeneracy of the genetic code, are not identical in sequence to the disclosed polynucleotides. Thus, the invention includes within its scope a polypeptide encoded by a polynucleotide having the sequence of any one of SEQ ID NOS:1-2396 or a variant thereof.

In general, the term “polypeptide” as used herein refers to both the full length polypeptide encoded by the recited polynucleotide, the polypeptide encoded by the gene represented by the recited polynucleotide, as well as portions or fragments thereof. “Polypeptides” also includes variants of the naturally occurring proteins, where such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, usually at least about 90%, and more usually at least about 98% sequence identity with a differentially expressed polypeptide of the invention, as measured by BLAST 2.0 using the parameters described above. The variant polypeptides can be naturally or non-naturally glycosylated, i.e., the polypeptide has a glycosylation pattern that differs from the glycosylation pattern found in the corresponding naturally occurring protein.

The invention also encompasses homologs of the disclosed polypeptides (or fragments thereof) where the homologs are isolated from other species, i.e. other animal or plant species, where such homologs, usually mammalian species, e.g. rodents, such as mice, rats; domestic animals, e.g., horse, cow, dog, cat; and humans. By “homolog” is meant a polypeptide having at least about 35%, usually at least about 40% and more usually at least about 60% amino acid sequence identity to a particular differentially expressed protein as identified above, where sequence identity is determined using the BLAST 2.0 algorithm, with the parameters described supra.

In general, the polypeptides of the subject invention are provided in a non-naturally occurring environment, e.g. are separated from their naturally occurring environment. In certain embodiments, the subject protein is present in a composition that is enriched for the protein as compared to a control. As such, purified polypeptide is provided, where by purified is meant that the protein is present in a composition that is substantially free of non-differentially expressed polypeptides, where by substantially free is meant that less than 90%, usually less than 60% and more usually less than 50% of the composition is made up of non-differentially expressed polypeptides.

Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence). Selection of amino acid alterations for production of variants can be based upon the accessibility (interior vs. exterior) of the amino acid (see, e.g., Go et al, Int. J. Peptide Protein Res. (1980) 15:21 1), the thermostability of the variant polypeptide (see, e.g., Querol et al., Prot. Eng. (1996) 9:265), desired glycosylation sites (see, e.g., Olsen and Thomsen, J. Gen. Microbiol. (1991) 137:579), desired disulfide bridges (see, e.g., Clarke et al., Biochemistry (1993) 32:4322; and Wakarchuk et al., Protein Eng. (1994) 7:1379), desired metal binding sites (see, e.g., Toma et al., Biochemistry (1991) 30:97, and Haezerbrouck et al., Protein Eng. (1993) 6:643), and desired substitutions with in proline loops (see, e.g., Masul et al., Appl. Env. Microbiol. (1994) 60:3579). Cysteine-depleted muteins can be produced as disclosed in U.S. Pat. No. 4,959,314.

Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a polynucleotide having a sequence of any SEQ ID NOS:1-2396, or a homolog thereof. The protein variants described herein are encoded by polynucleotides that are within the scope of the invention. The genetic code can be used to select the appropriate codons to construct the corresponding variants.

Computer-Related Embodiments

In general, a library of polynucleotides is a collection of sequence information, which information is provided in either biochemical form (e.g., as a collection of polynucleotide molecules), or in electronic form (e.g., as a collection of polynucleotide sequences stored in a computer-readable form, as in a computer system and/or as part of a computer program). The sequence information of the polynucleotides can be used in a variety of ways, e.g., as a resource for gene discovery, as a representation of sequences expressed in a selected cell type (e.g., cell type markers), and/or as markers of a given disease or disease state. In general, a disease marker is a representation of a gene product that is present in all cells affected by disease either at an increased or decreased level relative to a normal cell (e.g., a cell of the same or similar type that is not substantially affected by disease). For example, a polynucleotide sequence in a library can be a polynucleotide that represents an mRNA, polypeptide, or other gene product encoded by the polynucleotide, that is either overexpressed or underexpressed in a breast ductal cell affected by cancer relative to a normal (ie., substantially disease-free) breast cell.

The nucleotide sequence information of the library can be embodied in any suitable form, e.g., electronic or biochemical forms. For example, a library of sequence information embodied in electronic form comprises an accessible computer data file (or, in biochemical form, a collection of nucleic acid molecules) that contains the representative nucleotide sequences of genes that are differentially expressed (e.g., overexpressed or underexpressed) as between, for example, i) a cancerous cell and a normal cell; ii) a cancerous cell and a dysplastic cell; iii) a cancerous cell and a cell affected by a disease or condition other than cancer; iv) a metastatic cancerous cell and a normal cell and/or non-metastatic cancerous cell; v) a malignant cancerous cell and a non-malignant cancerous cell (or a normal cell) and/or vi) a dysplastic cell relative to a normal cell. Other combinations and comparisons of cells affected by various diseases or stages of disease will be readily apparent to the ordinarily skilled artisan. Biochemical embodiments of the library include a collection of nucleic acids that have the sequences of the genes in the library, where the nucleic acids can correspond to the entire gene in the library or to a fragment thereof, as described in greater detail below.

The polynucleotide libraries of the subject invention generally comprise sequence information of a plurality of polynucleotide sequences, where at least one of the polynucleotides has a sequence of any of SEQ ID NOS:1-2396. By plurality is meant at least 2, usually at least 3 and can include up to all of SEQ ID NOS:1-2396. The length and number of polynucleotides in the library will vary with the nature of the library, e.g., if the library is an oligonucleotide array, a cDNA array, a computer database of the sequence information, etc.

Where the library is an electronic library, the nucleic acid sequence information can be present in a variety of media. “Media” refers to a manufacture, other than an isolated nucleic acid molecule, that contains the sequence information of the present invention. Such a manufacture provides the genome sequence or a subset thereof in a form that can be examined by means not directly applicable to the sequence as it exists in a nucleic acid. For example, the nucleotide sequence of the present invention, e.g. the nucleic acid sequences of any of the polynucleotides of SEQ ID NOS:1-2396, can be recorded on computer readable media, e.g. any medium that can be read and accessed directly by a computer. Such media include, but are not limited to: magnetic storage media, such as a floppy disc, a hard disc storage medium, and a magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present sequence information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc. In addition to the sequence information, electronic versions of the libraries of the invention can be provided in conjunction or connection with other computer-readable information and/or other types of computer-readable files (e.g., searchable files, executable files, etc, including, but not limited to, for example, search program software, etc.).

By providing the nucleotide sequence in computer readable form, the information can be accessed for a variety of purposes. Computer software to access sequence information is publicly available. For example, the gapped BLAST (Altschul et al. Nucleic Acids Res. (1997) 25:33 89-3402) and BLAZE (Brutlag et al. Comp. Chem. (1993) 17:203) search algorithms on a Sybase system can be used to identify open reading frames (ORFs) within the genome that contain homology to ORFs from other organisms.

As used herein, “a computer-based system” refers to the hardware means, software means, and data storage means used to analyze the nucleotide sequence information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means can comprise any manufacture comprising a recording of the present sequence information as described above, or a memory access means that can access such a manufacture.

“Search means” refers to one or more programs implemented on the computer-based system, to compare a target sequence or target structural motif, or expression levels of a polynucleotide in a sample, with the stored sequence information. Search means can be used to identify fragments or regions of the genome that match a particular target sequence or target motif. A variety of known algorithms are publicly known and commercially available, e.g. MacPattern (EMBL), BLASTN and BLASTX (NCBI). A “target sequence” can be any polynucleotide or amino acid sequence of six or more contiguous nucleotides or two or more amino acids, preferably from about 10 to 100 amino acids or from about 30 to 300 nt A variety of comparing means can be used to accomplish comparison of sequence information from a sample (e.g., to analyze target sequences, target motifs, or relative expression levels) with the data storage means. A skilled artisan can readily recognize that any one of the publicly available homology search programs can be used as the search means for the computer based systems of the present invention to accomplish comparison of target sequences -and motifs. Computer programs to analyze expression levels in a sample and in controls are also known in the art.

A “target structural motif,” or “target motif,” refers to any rationally selected sequence or combination of sequences in which the sequence(s) are chosen based on a three-dimensional configuration that is formed upon the folding of the target motif, or on consensus sequences of regulatory or active sites. There are a variety of target motifs known in the art. Protein target motifs include, but arc not limited to, enzyme active sites and signal sequences. Nucleic acid target motifs include, but are not limited to, hairpin structures, promoter sequences and other expression elements such as binding sites for transcription factors.

A variety of structural formats for the input and output means can be used to input and output the information in the computer-based systems of the present invention. One format for an output means ranks the relative expression levels of different polynucleotides. Such presentation provides a skilled artisan with a ranking of relative expression levels to determine a gene expression profile.

As discussed above, the “library” of the invention also encompasses biochemical libraries of the polynucleotides of SEQ ID NOS:1-2396, e.g., collections of nucleic acids representing the provided polynucleotides. The biochemical libraries can take a variety of forms, e.g., a solution of cDNAs, a pattern of probe nucleic acids stably associated with a surface of a solid support (i.e., an array) and the like. Of particular interest are nucleic acid arrays in which one or more of SEQ ID NOS:1-2396 is represented on the array. By array is meant a an article of manufacture that has at least a substrate with at least two distinct nucleic acid targets on one of its surfaces, where the number of distinct nucleic acids can be considerably higher, typically being at least 10 nt, usually at least 20 nt and often at least 25 nt. A variety of different array formats have been developed and are known to those of skill in the art. The arrays of the subject invention find use in a variety of applications, including gene expression analysis, drug screening, mutation analysis and the like, as disclosed in the above-listed exemplary patent documents.

In addition to the above nucleic acid libraries, analogous libraries of polypeptides are also provided, where the where the polypeptides of the library will represent at least a portion of the polypeptides encoded by SEQ ID NOS:1-2396.

Utilities

Use of Polynucleotide Probes in Mapping, and in Tissue Profiling

Polynucleotide probes, generally comprising at least 12 contiguous nt of a polynucleotide as shown in the Sequence Listing, are used for a variety of purposes, such as chromosome mapping of the polynucleotide and detection of transcription levels. Additional disclosure about preferred regions of the disclosed polynucleotide sequences is found in the Examples. A probe that hybridizes specifically to a polynucleotide disclosed herein should provide a detection signal at least 5-, 10-, or 20-fold higher than the background hybridization provided with other unrelated sequences.

Detection of Expression Levels.

Nucleotide probes are used to detect expression of a gene corresponding to the provided polynucleotide. In Northern blots, mRNA is separated electrophoretically and contacted with a probe. A probe is detected as hybridizing to an mRNA species of a particular size. The amount of hybridization is quantitated to determine relative amounts of expression, for example under a particular condition. Probes are used for in situ hybridization to cells to detect expression. Probes can also be used in vivo for diagnostic detection of hybridizing sequences. Probes are typically labeled with a radioactive isotope. Other types of detectable labels can be used such as chromophores, fluors, and enzymes. Other examples of nucleotide hybridization assays are described in WO92/02526 and U.S. Pat. No. 5,124,246.

Alternatively, the Polymerase Chain Reaction (PCR) is another means for detecting small amounts of target nucleic acids (see, e.g., Mullis et al., Meth. Enzymol. (1987) 155:335; U.S. Pat. Nos. 4,683,195; and 4,683,202). Two primer polynucleotides nucleotides that hybridize with the target nucleic acids are used to prime the reaction. The primers can be composed of sequence within or 3′ and 5′ to the polynucleotides of the Sequence Listing. Alternatively, if the primers are 3′ and 5′ to these polynucleotides, they need not hybridize to them or the complements. After amplification of the target with a thermostable polymerase, the amplified target nucleic acids can be detected by methods known in the art, e.g., Southern blot. mRNA or cDNA can also be detected by traditional blotting techniques (e.g., Southern blot, Northern blot, etc.) described in Sambrook et al., “Molecular Cloning: A Laboratory Manual” (New York, Cold Spring Harbor Laboratory, 1989) (e.g., without PCR amplification). In general, mRNA or cDNA generated from mRNA using a polymerase enzyme can be purified and separated using gel electrophoresis, and transferred to a solid support, such as nitrocellulose. The solid support is exposed to a labeled probe, washed to remove any unhybridized probe, and duplexes containing the labeled probe are detected.

Mapping.

Polynucleotides of the present invention can be used to identify a chromosome on which the corresponding gene resides. Such mapping can be useful in identifying the function of the polynucleotide-related gene by its proximity to other genes with known function. Function can also be assigned to the polynucleotide-related gene when particular syndromes or diseases map to the same chromosome. For example, use of polynucleotide probes in identification and quantification of nucleic acid sequence aberrations is described in U.S. Pat. No. 5,783,387. An exemplary mapping method is fluorescence in situ hybridization (FISH), which facilitates comparative genomic hybridization to allow total genome assessment of changes in relative copy number of DNA sequences (see, e.g., Valdes et al., Methods in Molecular Biology (1997) 68: 1). Polynucleotides can also be mapped to particular chromosomes using, for example, radiation hybrids or chromosome-specific hybrid panels. See Leach et al., Advances in Genetics, (1995) 33:63-99; Walter et al., Nature Genetics (1994) 7:22; Walter and Goodfellow, Trends in Genetics (1992) 9:352. Panels for radiation hybrid mapping are available from Research Genetics, Inc., Huntsville, Ala., USA. Databases for markers using various panels are publicly available via the world wide web from the Stanford Genome Center and The Whitehead Institute for Biomedical Research/MIT Center for Genome Research. The statistical program RHMAP can be used to construct a map based on the data from radiation hybridization with a measure of the relative likelihood of one order versus another. RHMAP is available via the world wide web from the University of Michigan, Center for Statistical Genetics, Ann Arbor, Mich. In addition, commercial programs are available for identifying regions of chromosomes commonly associated with disease, such as cancer.

Tissue Typing or Profiling.

Expression of specific mRNA corresponding to the provided polynucleotides can vary in different cell types and can be tissue-specific. This variation of mRNA levels in different cell types can be exploited with nucleic acid probe assays to determine tissue types. For example, PCR, branched DNA probe assays, or blotting techniques utilizing nucleic acid probes substantially identical or complementary to polynucleotides listed in the Sequence Listing can determine the presence or absence of the corresponding cDNA or mRNA.

Tissue typing can be used to identify the developmental organ or tissue source of a metastatic lesion by identifying the expression of a particular marker of that organ or tissue. If a polynucleotide is expressed only in a specific tissue type, and a metastatic lesion is found to express that polynucleotide, then the developmental source of the lesion has been identified. Expression of a particular polynucleotide can be assayed by detection of either the corresponding mRNA or the protein product. As would be readily apparent to any forensic scientist, the sequences disclosed herein are useful in differentiating human tissue from non-human tissue. In particular, these sequences are useful to differentiate human tissue from bird, reptile, and amphibian tissue, for example.

Use of Polymorphisms.

A polynucleotide of the invention can be used in forensics, genetic analysis, mapping, and diagnostic applications where the corresponding region of a gene is polymorphic in the human population. Any means for detecting a polymorphism in a gene can be used, including, but not limited to electrophoresis of protein polymorphic variants, differential sensitivity to restriction enzyme cleavage, and hybridization to allele-specific probes.

Antibody Production

Expression products of a polynucleotide of the invention, as well as the corresponding mRNA, cDNA, or complete gene, can be prepared and used for raising antibodies for experimental, diagnostic, and therapeutic purposes. For polynucleotides to which a corresponding gene has not been assigned, this provides an additional method of identifying the corresponding gene. The polynucleotide or related cDNA is expressed as described above, and antibodies are prepared. These antibodies are specific to an epitope on the polypeptide encoded by the polynucleotide, and can precipitate or bind to the corresponding native protein in a cell or tissue preparation or in a cell-free extract of an in vitro expression system.

Methods for production of antibodies that specifically bind a selected antigen are well known in the art. Immunogens for raising antibodies can be prepared by mixing a polypeptide encoded by a polynucleotide of the invention with an adjuvant, and/or by making fusion proteins with larger imrnunogenic proteins. Polypeptides can also be covalently linked to other larger immunogenic proteins, such as keyhole limpet hemocyanin. Immunogens are typically administered intradermally, subcutaneously, or intramuscularly to experimental animals such as rabbits, sheep, and mice, to generate antibodies. Monoclonal antibodies can be Monoclonal antibodies can be generated by isolating spleen cells and fusing myeloma cells to form hybridomas. Alternatively, the selected polynucleotide is administered directly, such as by intramuscular injection, and expressed in vivo. The expressed protein generates a variety of protein-specific immune responses, including production of antibodies, comparable to administration of the protein.

Preparations of polyclonal and monoclonal antibodies specific for polypeptides encoded by a selected polynucleotide are made using standard methods known in the art. The antibodies specifically bind to epitopes present in the polypeptides encoded by polynucleotides disclosed in the Sequence Listing. Typically, at least 6, 8, 10, or 12 contiguous amino acids are required to form an epitope. Epitopes that involve non-contiguous amino acids may require a longer polypeptide, e.g., at least 15, 25, or 50 amino acids. Antibodies that specifically bind to human polypeptides encoded by the provided polypeptides should provide a detection signal at least 5-, 10-, or 20-fold higher than a detection signal provided with other proteins when used in Western blots or other immunochemical assays. Preferably, antibodies that specifically polypeptides of the invention do not bind to other proteins in immunochemical assays at detectable levels and can immunoprecipitate the specific polypeptide from solution.

The invention also contemplates naturally occurring antibodies specific for a polypeptide of the invention. For example, serum antibodies to a polypeptide of the invention in a human population can be purified by methods well known in the art, e.g., by passing antiserum over a C column to which the corresponding selected polypeptide or fusion protein is bound. The bound antibodies can then be eluted from the column, for example using a buffer with a high salt concentration.

In addition to the antibodies discussed above, the invention also contemplates genetically engineered antibodies, antibody derivatives (e.g., single chain antibodies, antibody fragments (e.g., Fab, etc.)), according to methods well known in the art.

Polynucleotides or Arrays for Diagnostics

Polynucleotide arrays provide a high throughput technique that can assay a large number of polynucleotide sequences in a sample. This technology can be used as a diagnostic and as a tool to test for differential expression, e.g., to determine function of an encoded protein. Arrays can be created by spotting polynucleotide probes onto a substrate (e.g., glass, nitrocelllose, etc.) in a two-dimensional matrix or array having bound probes. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Techniques for constructing arrays and methods of using these arrays are described in EP 799 897; WO 97/29212; WO 97/27317; EP 785 280; WO 97/02357; U.S. Pat. Nos. 5,593,839; 5,578,832; EP 728 520; U.S. Pat. No. 5,599,695; EP 721 016; U.S. Pat. No. 5,556,752; WO 95/22058; and U.S. Pat. No. 5,631,734. Arrays can be used to, for example, examine differential expression of genes and can be used to determine gene function. For example, arrays can be used to detect differential expression of a polynucleotide between a test cell and control cell (e.g., cancer cells and normal cells). For example, high expression of a particular message in a cancer cell, which is not observed in a corresponding normal cell, can indicate a cancer specific gene product. Exemplary uses of arrays are further described in, for example, Pappalarado et al., Sem. Radiation Oncol. (1998) 8:217; and Ramsay Nature Biotechnol. (1998) 16:40.

Differential Expression in Diagnosis

The polynucleotides of the invention can also be used to detect differences in expression levels between two cells, e.g., as a method to identify abnormal or diseased tissue in a human. For polynucleotides corresponding to profiles of protein families, the choice of tissue can be selected according to the putative biological function. In general, the expression of a gene corresponding to a specific polynucleotide is compared between a first tissue that is suspected of being diseased and a second, normal tissue of the human. The tissue suspected of being abnormal or diseased can be derived from a different tissue type of the human, but preferably it is derived from the same tissue type; for example an intestinal polyp or other abnormal growth should be compared with normal intestinal tissue. The normal tissue can be the same tissue as that of the test sample, or any normal tissue of the patient, especially those that express the polynucleotide-related gene of interest (e.g., brain, thymus, testis, heart, prostate, placenta, spleen, small intestine, skeletal muscle, pancreas, and the mucosal lining of the colon). A difference between the polynucleotide-related gene, mRNA, or protein in the two tissues which are compared, for example in molecular weight, amino acid or nucleotide sequence, or relative abundance, indicates a change in the gene, or a gene which regulates it, in the tissue of the human that was suspected of being diseased. Examples of detection of differential expression and its use in diagnosis of cancer are described in U.S. Pat. Nos. 5,688,641 and 5,677,125.

A genetic predisposition to disease in a human can also be detected by comparing expression levels of an mRNA or protein corresponding to a polynucleotide of the invention in a fetal tissue with levels associated in normal fetal tissue. Fetal tissues that are used for this purpose include, but are not limited to, amniotic fluid, chorionic villi, blood, and the blastomere of an in vitro-fertilized embryo. The comparable normal polynucleotide-related gene is obtained from any tissue. The mRNA or protein is obtained from a normal tissue of a human in which the polynucleotide-related gene is expressed. Differences such as alterations in the nucleotide sequence or size of the same product of the fetal polynucleotide-related gene or mRNA, or alterations in the molecular weight, amino acid sequence, or relative abundance of fetal protein, can indicate a germiline mutation in the polynucleotide-related gene of the fetus, which indicates a genetic predisposition to disease. In general, diagnostic, prognostic, and other methods of the invention based on differential expression involve detection of a level or amount of a gene product, particularly a differentially expressed gene product, in a test sample obtained from a patient suspected of having or being susceptible to a disease (e.g., breast cancer, lung cancer, colon cancer and/or metastatic forms thereof), and comparing the detected levels to those levels found in normal cells (e.g., cells substantially unaffected by cancer) and/or other control cells (e.g., to differentiate a cancerous cell from a cell affected by dysplasia). Furthermore, the severity of the disease can be assessed by comparing the detected levels of a differentially expressed gene product with those levels detected in samples representing the levels of differentially gene product associated with varying degrees of severity of disease. It should be noted that use of the term “diagnostic” herein is not necessarily meant to exclude “prognostic” or “prognosis,” but rather is used as a matter of convenience.

The term “differentially expressed gene” is generally intended to encompass a polynucleotide that can, for example, include an open reading frame encoding a gene product (e.g., a polypeptide), and/or introns of such genes and adjacent 5′ and 3′ non-coding nucleotide sequences involved in the regulation of expression, up to about 20 kb beyond the coding region, but possibly further in either direction. The gene can be introduced into an appropriate vector for extrachromosomal maintenance or for integration into a host genome. In general, a difference in expression level associated with a decrease in expression level of at least about 25%, usually at least about 50% to 75%, more usually at least about 90% or more is indicative of a differentially expressed gene of interest, i.e., a gene that is underexpressed or down-regulated in the test sample relative to a control sample. Furthermore, a difference in expression level associated with an increase in expression of at least about 25%, usually at least about 50% to 75%, more usually at least about 90% and can be at least about 1 I2-fold, usually at least about 2-fold to about 10-fold, and can be about 100-fold to about 1,000-fold increase relative to a control sample is indicative of a differentially expressed gene of interest, i.e., an overexpressed or up-regulated gene.

“Differentially expressed polynucleotide” as used herein means a nucleic acid molecule (RNA or DNA) comprising a sequence that represents a differentially expressed gene, e.g., the differentially expressed polynucleotide comprises a sequence (e.g., an open reading frame encoding a gene product) that uniquely identifies a differentially expressed gene so that detection of the differentially expressed polynucleotide in a sample is correlated with the presence of a differentially expressed gene in a sample. “Differentially expressed polynucleotides” is also meant to encompass fragments of the disclosed polynucleotides, e.g., fragments retaining biological activity, as well as nucleic acids homologous, substantially similar, or substantially identical (e.g., having about 90% sequence identity) to the disclosed polynucleotides.

“Diagnosis” as used herein generally includes determination of a subject's susceptibility to a disease or disorder, determination as to whether a subject is presently affected by a disease or disorder, as well as to the prognosis of a subject affected by a disease or disorder (e.g., identification of pre-metastatic or metastatic cancerous states, stages of cancer, or responsiveness of cancer to therapy). The present invention particularly encompasses diagnosis of subjects in the context of breast cancer (e.g., carcinoma in situ (e.g., ductal carcinoma in situ), estrogen receptor (ER)-positive breast cancer, ER-negative breast cancer, or other forms and/or stages of breast cancer), lung cancer (e.g., small cell carcinoma, non-small cell carcinoma, mesothelioma, and other forms and/or stages of lung cancer), and colon cancer (e.g., adenomatous polyp, colorectal carcinoma, and other forms and/or stages of colon cancer).

“Sample” or “biological sample” as used throughout here are generally meant to refer to samples of biological fluids or tissues, particularly samples obtained from tissues, especially from cells of the type associated with the disease for which the diagnostic application is designed (e.g., ductal adenocarcinoma), and the like. “Samples” is also meant to encompass derivatives and fractions of such samples (e.g., cell lysates). Where the sample is solid tissue, the cells of the tissue can be dissociated or tissue sections can be analyzed.

Methods of the subject invention useful in diagnosis or prognosis typically involve comparison of the abundance of a selected differentially expressed gene product in a sample of interest with that of a control to determine any relative differences in the expression of the gene -product, where the difference can be measured qualitatively and/or quantitatively. Quantitation can be accomplished, for example, by comparing the level of expression product detected in the sample with the amounts of product present in a standard curve. A comparison can be made visually; by using a technique such as densitometry, with or without computerized assistance; by preparing a representative library of cDNA clones of mRNA isolated from a test sample, sequencing the clones in the library to determine that number of cDNA clones corresponding to the same gene product, and analyzing the number of clones corresponding to that same gene product relative to the number of clones of the same gene product in a control sample; or by using an array to detect relative levels of hybridization to a selected sequence or set of sequences, and comparing the hybridization pattern to that of a control. The differences in expression are then correlated with the presence or absence of an abnormal expression pattern. A variety of different methods for determining the nucleic acid abundance in a sample are known to those of skill in the art (see, e.g., WO 97/27317).

In general, diagnostic assays of the invention involve detection of a gene product of a the polynucleotide sequence (e.g., mRNA or polypeptide) that corresponds to a sequence of SEQ ID NOS:1-2396 The patient from whom the sample is obtained can be apparently healthy, susceptible to disease (e.g., as determined by family history or exposure to certain environmental factors), or can already be identified as having a condition in which altered expression of a gene product of the invention is implicated.

Diagnosis can be determined based on detected gene product expression levels of a gene product encoded by at least one, preferably at least two or more, at least 3 or more, or at least 4 or more of the polynucleotides having a sequence set forth in SEQ ID NOS:1-2396, and can involve detection of expression of genes corresponding to all of SEQ ID NOS:1-2396 and/or additional sequences that can serve as additional diagnostic markers and/or reference sequences. Where the diagnostic method is designed to detect the presence or susceptibility of a patient to cancer, the assay preferably involves detection of a gene product encoded by a gene corresponding to a polynucleotide that is differentially expressed in cancer. Examples of such differentially expressed polynucleotides are described in the Examples below. Given the provided polynucleotides and information regarding their relative expression levels provided herein, assays using such polynucleotides and detection of their expression levels in diagnosis and prognosis will be readily apparent to the ordinarily skilled artisan.

Any of a variety of detectable labels can be used in connection with the various embodiments of the diagnostic methods of the invention. Suitable detectable labels include fluorochromes,(e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g. ³²P, 35S, ³H, etc.), and the like. The detectable label can involve a two stage systems (e.g., biotin-avidin, hapten-anti-hapten antibody, etc.).

Reagents specific for the polynucleotides and polypeptides of the invention, such as antibodies and nucleotide probes, can be supplied in a kit for detecting the presence of an expression product in a biological sample. The kit can also contain buffers or labeling components, as well as instructions for using the reagents to detect and quantify expression products in the biological sample. Exemplary embodiments of the diagnostic methods of the invention are described below in more detail.

Polypeptide Detection in Diagnosis.

In one embodiment, the test sample is assayed for the level of a differentially expressed polypeptide. Diagnosis can be accomplished using any of a number of methods to determine the absence or presence or altered amounts of the differentially expressed polypeptide in the test sample. For example, detection can utilize staining of cells or histological sections with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

mRNA Detection.

The diagnostic methods of the invention can also or alternatively involve detection of mRNA encoded by a gene corresponding to a differentially expressed polynucleotides of the invention. Any suitable qualitative or quantitative methods known in the art for detecting specific mRNAs can be used. mRNA can be detected by, for example, in situ hybridization in tissue -sections, by reverse transcriptase-PCR, or in Northern blots containing poly A+ mRNA. One of skill in the art can readily use these methods to determine differences in the size or amount of mRNA transcripts between two samples. mRNA expression levels in a sample can also be determined by generation of a library of expressed sequence tags (ESTs) from the sample, where the EST library is representative of sequences present in the sample (Adams, et al., (1991) Science 252:1651). Enumeration of the relative representation of ESTs within the library can be used to approximate the relative representation of the gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein. Alternatively, gene expression in a test sample can be performed using serial analysis of gene expression (SAGE) methodology (e.g., Velculescu et al., Science (1995) 270:484) or differential display (DD) methodology (see, e.g. U.S. Pat. No. 5,776,683; and U.S. Pat. No. 5,807,680).

Alternatively, gene expression can be analyzed using hybridization analysis. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Use of a Single Gene in Diagnostic Applications.

The diagnostic methods of the invention can focus on the expression of a single differentially expressed gene. For example, the diagnostic method can involve detecting a differentially expressed gene, or a polymorphism of such a gene (e.g., a polymorphism in an coding region or control region), that is associated with disease.

Disease-associated polymorphisms can include deletion or truncation of the gene, mutations that alter expression level and/or affect activity of the encoded protein, etc.

A number of methods are available for analyzing nucleic acids for the presence of a specific sequence, e.g. a disease associated polymorphism. Where large amounts of DNA are available, genomic DNA is used directly. Alternatively, the region of interest is cloned into a suitable vector and grown in sufficient quantity for analysis. Cells that express a differentially expressed gene can be used as a source of mRNA, which can be assayed directly or reverse transcribed into cDNA for analysis. The nucleic acid can be amplified by conventional techniques, such as the polymerase chain reaction (PCR), to provide sufficient amounts for analysis, and a detectable label can be included in the amplification reaction (e.g., using a detectably labeled primer or detectably labeled oligonucleotides) to facilitate detection. Alternatively, various methods are also known in the art that utilize oligonucleotide ligation as a means of detecting polymorphisms, see e.g., Riley et al., Nucl. Acids Res. (1990) 18:2887; and Delahunty et al., Am. J. Hum. Genet. (1996) 58:1239.

The amplified or cloned sample nucleic acid can be analyzed by one of a number of methods known in the art. The nucleic acid can be sequenced by dideoxy or other methods, and the sequence of bases compared to a selected sequence, e.g., to a wild-type sequence. Hybridization with the polymorphic or variant sequence can also be used to determine its presence in a sample (e.g. by Southern blot, dot blot, etc.). The hybridization pattern of a polymorphic or variant sequence and a control sequence to an array of oligonucleotide probes immobilized on a solid support, as described in U.S. Pat. No. 5,445,934, or in WO 95/35505, can also be used as a means of identifwwing polymorphic or variant sequences associated with disease. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Alternatively, where a polymorphism creates or destroys a recognition site 355 for a restriction endonuclease, the sample is digested with that endonuclease, and the products size fractionated to determine whether the fragment was digested. Fractionation is performed by gel or capillary electrophoresis, particularly acrylamide or agarose gels.

Screening for mutations in a gene can be based on the functional or antigenic characteristics of the protein. Protein truncation assays are useful in detecting deletions that can affect the biological activity of the protein. Various immunoassays designed to detect polymorphisms in proteins can be used in screening. Where many diverse genetic mutations lead to a particular disease phenotype, functional protein assays have proven to be effective screening tools. The activity of the encoded protein can be determined by comparison with the wild-type protein.

Pattern Matching in Diagnosis Using Arrays.

In another embodiment, the diagnostic and/or prognostic methods of the invention involve detection of expression of a selected set of genes in a test sample to produce a test expression pattern (TEP). The TEP is compared to a reference expression pattern (REP), which is generated by detection of expression of the selected set of genes in a reference sample (e.g., a positive or negative control sample). The selected set of genes includes at least one of the genes of the invention, which genes correspond to the polynucleotide sequences of SEQ ID NOS:1-2396. Of particular interest is a selected set of genes that includes gene differentially expressed in the disease for which the test sample is to be screened.

“Reference sequences” or “reference polynucleotides” as used herein in the context of differential gene expression analysis and diagnosis/prognosis refers to a selected set of polynucleotides, which selected set includes at least one or more of the differentially expressed polynucleotides described herein. A plurality of reference sequences, preferably comprising positive and negative control sequences, can be included as reference sequences. Additional suitable reference sequences are found in GenBank, Unigene, and other nucleotide sequence databases (including, e.g., expressed sequence tag (EST), partial, and full-length sequences).

“Reference array” means an array having reference sequences for use in hybridization with a sample, where the reference sequences include all, at least one of, or any subset of the differentially expressed polynucleotides described herein. Usually such an array will include at least 3 different reference sequences, and can include any one or all of the provided differentially expressed sequences. Arrays of interest can further comprise sequences, including polymorphisms, of other genetic sequences, particularly other sequences of interest for screening for a disease or disorder (e.g., cancer, dysplasia, or other related or unrelated diseases, disorders, or conditions). The oligonucleotide sequence on the array will usually be at least about 12 nt in length, and can be of about the length of the provided sequences, or can extend into the flanking regions to generate fragments of 100 nt to 200 nt in length or more. Reference arrays can be produced according to any suitable methods known in the art. For example, methods of producing large arrays of oligonucleotides are described in U.S. Pat. Nos. 5,134,854, and 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

A “reference expression pattern” or “REP” as used herein refers to the relative levels of expression of a selected set of genes, particularly of differentially expressed genes, that is associated with a selected cell type, e.g., a normal cell, a cancerous cell, a cell exposed to an environmental stimulus, and the like. A “test expression pattern” or “TEP” refers to relative levels of expression of a selected set of genes, particularly of differentially expressed genes, in a test sample (e.g. a cell of unknown or suspected disease state, from which mRNA is isolated).

REPs can be generated in a variety of ways according to methods well known in the art. For example, REPs can be generated by hybridizing a control sample to an array having a selected set of polynucleotides (particularly a selected set of differentially expressed polynucleotides), acquiring the hybridization data from the array, and storing the data in a format that allows for ready comparison of the REP with a TEP. Alternatively, all expressed sequences in a control sample can be isolated and sequenced, e.g., by isolating mRNA from a control sample, converting the mRNA into cDNA, and sequencing the cDNA. The resulting sequence information roughly or precisely reflects the identity and relative number of expressed sequences in the sample. The sequence information can then be stored in a format (e.g., a computer-readable format) that allows for ready comparison of the REP with a TEP. The REP can be normalized prior to or after data storage, and/or can be processed to selectively remove sequences of expressed genes that are of less interest or that might complicate analysis (e.g., some or all of the sequences associated with housekeeping genes can be eliminated from REP data).

TEPs can be generated in a manner similar to REPs, e.g., by hybridizing a test sample to an array having a selected set of polynucleotides, particularly a selected set of differentially expressed polynucleotides, acquiring the hybridization data from the array, and storing the data in a format that allows for ready comparison of the TEP with a REP. The REP and TEP to be used in a comparison can be generated simultaneously, or the TEP can be compared to previously generated and stored REPs.

In one embodiment of the invention, comparison of a TEP with a REP involves hybridizing a test sample with a reference array, where the reference array has one or more reference sequences for use in hybridization with a sample. The reference sequences include all, at least one of, or any subset of the differentially expressed polynucleotides described herein. Hybridization data for the test sample is acquired, the data normalized, and the produced TEP compared with a REP generated using an array having the same or similar selected set of differentially expressed polynucleotides. Probes that correspond to sequences differentially expressed between the two samples will show decreased or increased hybridization efficiency for one of the samples relative to the other.

Methods for collection of data from hybridization of samples with a reference arrays are well known in the art. For example, the polynucleotides of the reference and test samples can be generated using a detectable fluorescent label, and hybridization of the polynucleotides in the samples detected by scanning the microarrays for the presence of the detectable label using, for example, a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734.A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample (e.g., a test sample) is compared to the fluorescent signal from another sample (e.g., a reference sample), and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

In general, the test sample is classified as having a gene expression profile corresponding to that associated with a disease or non-disease state by comparing the TEP generated from the test sample to one or more REPs generated from reference samples (e.g., from samples associated with cancer or specific stages of cancer, dysplasia, samples affected by a disease other than cancer, normal samples, etc.). The criteria for a match or a substantial match between a TEP and a REP include expression of the same or substantially the same set of reference genes, as well as expression of these reference genes at substantially the same levels (e.g., no significant difference between the samples for a signal associated with a selected reference sequence after normalization of the samples, or at least no greater than about 25% to about 40% difference in signal strength for a given reference sequence. In general, a pattern match between a TEP and a REP includes a match in expression, preferably a match in qualitative or quantitative expression level, of at least one of, all or any subset of the differentially expressed genes of the invention.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

Targets for Inhibtion of Tumor Growth.

The polynucleotides of the invention can correspond to therapeutic targets, and modulation of expression and/or activity of these targets can provide for inhibition of tumor growth. For example, where overexpression of a gene is associated with tumor growth or metastasis, the gene product is a suitable for target for inhibition of its expression and/or activity to facilitate inihibtion of tumor growth or metastasis. The polynucleotides of the invention can correspond to such genes, and thus in some embodiments the antisense of these polynucleotides can be used to inhibit the expression of the gene and its corresponding gene product.

Diagnosis Prognosis and Management of Cancer

The polynucleotides of the invention and their gene products are of particular interest as genetic or biochemical markers (e.g., in blood or tissues) that will detect the earliest changes along the carcinogenesis pathway and/or to monitor the efficacy of various therapies and preventive interventions. For example, the level of expression of certain polynucleotides can be indicative of a poorer prognosis, and therefore warrant more aggressive chemo- or radio-therapy for a patient or vice versa. The correlation of novel surrogate tumor specific features with response to treatment and outcome in patients can define prognostic indicators that allow the design of tailored therapy based on the molecular profile of the tumor. These therapies include antibody targeting and gene therapy. Determining expression of certain polynucleotides and comparison of a patients profile with known expression in normal tissue and variants of the disease allows a determination of the best possible treatment for a patient, both in terms of specificity of treatment and in terms of comfort level of the patient. Surrogate tumor markers, such as polynucleotide expression, can also be used to better classify, and thus diagnose and treat, different forms and disease states of cancer. Two classifications widely used in oncology that can benefit from identification of the expression levels of the polynucleotides of the invention are staging of the cancerous disorder, and grading the nature of the cancerous tissue.

The polynucleotides of the invention can be useful to monitor patients having or susceptible to cancer to detect potentially malignant events at a molecular level before they are detectable at a gross morphological level. Furthermore, a polynucleotide of the invention identified as important for one type of cancer can also have implications for development or risk of development of other types of cancer, e.g., where a polynucleotide is differentially expressed across various cancer types. Thus, for example, expression of a polynucleotide that has clinical implications for metastatic colon cancer can also have clinical implications for stomach cancer or endometrial cancer.

Staging.

Staging is a process used by physicians to describe how advanced the cancerous state is in a patient. Staging assists the physician in determining a prognosis, planning treatment and evaluating the results of such treatment. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage TV, the most advanced stage.

The polynucleotides of the invention can facilitate fine-tuning of the staging process by identifying markers for the aggresivity of a cancer, e.g. the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

Grading of Cancers.

Grade is a term used to describe how closely a tumor resembles normal tissue of its same type. The microscopic appearance of a tumor is used to identify tumor grade based on parameters such as cell morphology, cellular organization, and other markers of differentiation. As a general rule, the grade of a tumor corresponds to its rate of growth or aggressiveness, with undifferentiated or high-grade tumors being more aggressive than well differentiated or low-grade tumors. The following guidelines are generally used for grading tumors: 1) GX Grade cannot be assessed; 2) G1 Well differentiated; G2 Moderately well differentiated; 3) G3 Poorly differentiated; 4) G4 Undifferentiated. The polynucleotides of the invention can be especially valuable in determining the grade of the tumor, as they not only can aid in determining the differentiation status of the cells of a tumor, they can also identify factors other than differentiation that are valuable in determining the aggressiveness of a tumor, such as metastatic potential.

Detection of Lung Cancer.

The polynucleotides of the invention can be used to detect lung cancer in a subject. Although there are more than a dozen different kinds of lung cancer, the two main types of lung cancer are small cell and nonsmall cell, which encompass about 90% of all lung cancer cases. Small cell carcinoma (also called oat cell carcinoma) usually starts in one of the larger bronchial tubes, grows fairly rapidly, and is likely to be large by the time of diagnosis. Nonsmall cell lung cancer (NSCLC) is made up of three general subtypes of lung cancer. Epidermoid carcinoma (also called squamous cell carcinoma) usually starts in one of the larger bronchial tubes and grows relatively slowly. The size of these tumors can range from very small to quite large.

Adenocarcinoma starts growing near the outside surface of the lung and can vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

The polynucleotides of the invention, e.g., polynucleotides differentially expressed in normal cells versus cancerous lung cells (e.g., tumor cells of high or low metastatic potential) or between types of cancerous lung cells (e.g., high metastatic versus low metastatic), can be used to distinguish types of lung cancer as well as identifying traits specific to a certain patient's cancer and selecting an appropriate therapy. For example, if the patient's biopsy expresses a polynucleotide that is associated with a low metastatic potential, it may justify leaving a larger portion of the patient's lung in surgery to remove the lesion. Alternatively, a smaller lesion with expression of a polynucleotide that is associated with high metastatic potential may justify a more radical removal of lung tissue and/or the surrounding lymph nodes, even if no metastasis can be identified through pathological examination.

Detection of Breast Cancer.

The majority of breast cancers are adenocarcinomas subtypes, which can be summarized as follows: 1) ductal carcinoma in situ (DCIS), including comedocarcinoma; 2) infiltrating (or invasive) ductal carcinoma (lDC); 3) lobular carcinoma in situ (LCIS); 4) infiltrating (or invasive) lobular carcinoma (ILC); 5) inflammatory breast cancer; 6) medullary carcinoma; 7) mucinous carcinoma; 8) Paget's disease of the nipple; 9) Phyllodes tumor; and 10) tubular carcinoma;

The expression of polynucleotides of the invention can be used in the diagnosis and management of breast cancer, as well as to distinguish between types of breast cancer. Detection of breast cancer can be determined using expression levels of any of the appropriate polynucleotides of the invention, either alone or in combination. Determination of the aggressive nature and/or the metastatic potential of a breast cancer can also be determined by comparing levels of one or more polynucleotides of the invention and comparing levels of another sequence known to vary in cancerous tissue, e.g. ER expression. In addition, development of breast cancer can be detected by examining the ratio of expression of a differentially expressed polynucleotide to the levels of steroid hormones (e.g., testosterone or estrogen) or to other hormones (e.g., growth hormone, insulin). Thus expression of specific marker polynucleotides can be used to discriminate between normal and cancerous breast tissue, to discriminate between breast cancers with different cells of origin, to discriminate between breast cancers with different potential metastatic rates, etc.

Detection of Colon Cancer.

The polynucleotides of the invention exhibiting the appropriate expression pattern can be used to detect colon cancer in a subject. Colorectal cancer is one of the most common neoplasms in humans and perhaps the most frequent form of hereditary neoplasia. Prevention and early detection are key factors in controlling and curing colorectal cancer. Colorectal cancer begins as polyps, which are small, benign growths of cells that form on the inner lining of the colon. Over a period of several years, some of these polyps accumulate additional mutations and become cancerous. Multiple familial colorectal cancer disorders have been identified, which are summarized as follows: 1) Familial adenomatous polyposis (FAP); 2) Gardner's syndrome; 3) Hereditary nonpolyposis colon cancer (HNPCC); and 4) Familial colorectal cancer in Ashkenazi Jews. The expression of appropriate polynucleotides of the invention can be used in the diagnosis, prognosis and management of colorectal cancer. Detection of colon cancer can be determined using expression levels of any of these sequences alone or in combination with the levels of expression. Determination of the aggressive nature and/or the metastatic potential of a colon cancer can be determined by comparing levels of one or more polynucleotides of the invention and comparing total levels of another sequence known to vary in cancerous tissue, e.g., expression of p53, DCC ras, lor FAP (see, e.g., Fearon E R, et al., Cell (1990) 61 (5):759; Hamilton S R et al., Cancer (1993) 72:957; Bodmer W, et al., Nat Genet. (1994) 4(3):217; Fearon E R, Ann N Y Acad Sci. (1995) 768:101). For example, development of colon cancer can be detected by examining the ratio of any of the polynucleotides of the invention to the levels of oncogenes (e.g. ras) or tumor suppressor genes (e.g. FAP or p53). Thus expression of specific marker polynucleotides can be used to discriminate between normal and cancerous colon tissue, to discriminate between colon cancers with different cells of origin, to discriminate between colon cancers with different potential metastatic rates, etc.

Detection of Prostate Cancer.

The polynucleotides and their corresponding genes and gene products exhibiting the appropriate differential expression pattern can be used to detect prostate cancer in a subject. Over 95% of primary prostate cancers are adenocarcinomas. Signs and symptoms may include: frequent urination, especially at night, inability to urinate, trouble starting or holding back urination, a weak or interrupted urine flow and frequent pain or stiffness in the lower back, hips or upper thighs.

Many of the signs and symptoms of prostate cancer can be caused by a variety of other non-cancerous conditions. For example, one common cause of many of these signs and symptoms is a condition called benign prostatic hypertrophy, or BPH. In BPH, the prostate gets bigger and may block the flow or urine or interfere with sexual function. The methods and compositions of the invention can be used to distinguish between prostate cancer and such non-cancerous conditions. The methods of the invention can be used in conjunction with conventional methods of diagnosis, e.g., digital rectal exam and/or detection of the level of prostate specific antigen (PSA), a substance produced and secreted by the prostate.

Use of Polynucleotides to Screen for Peptide Analogs and Antagonists

Polypeptides encoded by the instant polynucleotides and corresponding full length genes can be used to screen peptide libraries to identify binding partners, such as receptors, from among the encoded polypeptides. Peptide libraries can be synthesized according to methods known in the art (see, e.g., U.S. Pat. No. 5,010,175 , and WO 91/17823). Agonists or antagonists of the polypeptides if the invention can be screened using any available method known in the art, such as signal transduction, antibody binding, receptor binding, mitogenic assays, chemotaxis assays, etc. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of binding irreversibly to the polypeptide can be added in concentrations on the order of the native concentration.

Such screening and experimentation can lead to identification of a novel polypeptide binding partner, such as a receptor, encoded by a gene or a cDNA corresponding to a polynucleotide of the invention, and at least one peptide agonist or antagonist of the novel binding partner. Such agonists and antagonists can be used to modulate, enhance, or inhibit receptor function in cells to which the receptor is native, or in cells that possess the receptor as a result of genetic engineering. Further, if the novel receptor shares biologically important characteristics with a known receptor, information about agonist/antagonist binding can facilitate development of improved agonists/antagonists of the known receptor.

Pharmaceutical Compositions and Therapeutic Uses

Pharmaceutical compositions of the invention can comprise polypeptides, antibodies, or polynucleotides (including antisense nucleotides and ribozymes) of the claimed invention in a therapeutically effective amount. The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subjects size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington 's Pharmaceutical Sciences (Mack Pub. Co., N.J. 1991).

Delivery Methods.

Once formulated, the compositions of the invention can be (1) administered directly to the subject (e.g., as polynucleotide or polypeptides); or (2) delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Once a gene corresponding to a polynucleotide of the invention has been found to correlate with a proliferative disorder, such as neoplasia, dysplasia, and hyperplasia, the disorder can be amenable to treatment by administration of a therapeutic agent based on the provided polynucleotide, corresponding polypeptide or other corresponding molecule (e.g., antisense, ribozyme, etc.).

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic compositions agents of the invention includes local or systemic administration, including injection, oral administration, particle gun or catheterized administration, and topical administration. Preferably, the therapeutic polynucleotide composition contains an expression construct comprising a promoter operably linked to a polynucleotide of at least 12, 22, 25, 30, or 35 contiguous nt of the polynucleotide disclosed herein. Various methods can be used to administer the therapeutic composition directly to a specific site in the body. For example, a small metastatic lesion is located and the therapeutic composition injected several times in several different locations within the body of tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. The antisense composition is directly administered to the surface of the tumor, for example, by topical application of the composition. X-ray imaging is used to assist in certain of the above delivery methods. Receptor-mediated targeted delivery of therapeutic compositions containing an antisense polynucleotide, subgenomic polynucleotides, or antibodies to specific tissues can also be used.

Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g., for enhancing or inhibiting levels of the encoded gene product) and efficacy of transformation and expression are considerations which will affect the dosage required for ultimate efficacy of the antisense subgenomic polynucleotides. Where greater expression is desired over a larger area of tissue, larger amounts of antisense subgenomic polynucleotides or the same amounts readministered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect. For polynucleotide related genes encoding polypeptides or proteins with anti-inflammatory activity, suitable use, doses, and administration are described in U.S. Pat. No. 5,654,173.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA(see, e.g., Wu, J. Biol. Chem. (1989) 264:16985); eukaryotic cell delivery vehicles cells (see, e.g., U.S. Pat. No. 5,814,482; WO 95/07994; WO 96/17072;WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell Biol. (1994) 14:2411, and in Woffendin, Proc. Natl. Acad. Sci. (1994) 91:1581.

Further non-viral delivery suitable for use includes mechanical delivery systems such as the approach described in Woffendin et al., Proc. Natl. Acad. Sci. USA (1994) 91 (24):11581. Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

The present invention will now be illustrated by reference to the following examples which set forth particularly advantageous embodiments. However, it should be noted that these embodiments are illustrative and are not to be construed as restricting the invention in any way.

### EXAMPLES

The following examples are offered primarily for purposes of illustration. It will be readily apparent to those skilled in the art that the formulations, dosages, methods of administration, and other parameters of this invention may be further modified or substituted in various ways without departing from the spirit and scope of the invention.

### Example 1

### Source of Biological Materials and Overview of Novel Polynucleotides Expressed by the Biological Materials

cDNA libraries were constructed from mRNA isolated from the cell lines indicated in Table 4. The specific library from which any polynucleotide was isolated is indicated in Table 1, with the number of the entry under the “LIBRARY” column correlating to the library number in Table 4. Polynucleotides expressed by the selected cell lines were isolated and analyzed; the sequences of these polynucleotides were about 275-300 nucleotides in length.

The sequences of the isolated polynucleotides were first masked to eliminate low complexity sequences using the XBLAST masking program (Clayerie “Effective Large-Scale Sequence Similarity Searches,” In: Computer Methods for Macromolecular Sequence Analysis, Doolittle, ed., Meth. Enzymol. 266:212-227 Academic Press, NY, NY (1996); see particularly Clayerie, in “Automated DNA Sequencing and Analysis Techniques” Adams et al., eds., Chap. 36, p. 267 Academic Press, San Diego, 1994 and Clayerie et al. Comput. Chem. (1993) 17:191). Generally, masking does not influence the final search results, except to eliminate sequences of relative little interest due to their low complexity, and to eliminate multiple “hits” based on similarity to repetitive regions common to multiple sequences, e.g., Alu repeats. The remaining sequences were then used in a BLASTN vs. GenBank search; sequences that exhibited greater than 70% overlap, 99% identity, and a p value of less than 1×10⁻⁴⁰ were discarded. Sequences from this search also were discarded if the inclusive parameters were met, but the sequence was ribosomal or vector-derived.

The resulting sequences from the previous search were classified into three groups (1, 2 and 3 below) and searched in a BLASTX vs. NRP (non-redundant proteins) database search: (1) unknown (no hits in the GenBank search), (2) weak similarity (greater than 45% identity and p value of less than 1×10⁻⁵), and (3) high similarity (greater than 60% overlap, greater than 80% identity, and p value less than 1×10⁻⁵). Sequences having greater than 70% overlap, greater than 99% identity, and p value of less than 1×10⁻⁴⁰ were discarded.

The remaining sequences were classified as unknown (no hits), weak similarity, and high similarity (parameters as above). Two searches were performed on these sequences. First, a BLAST vs. EST database search was performed and sequences with greater than 99% overlap, greater than 99% similarity and a p value of less than 1×10⁻⁴⁰ were discarded. Sequences with a p value of less than 1×10⁻⁶⁵ when compared to a database sequence of human origin were also excluded. Second, a BLASTN vs. Patent GeneSeq database was performed and sequences having greater than 99% identity, p value less than 1×10⁻⁴⁰, and greater than 99% overlap were discarded.

The remaining sequences were subjected to screening using other rules and redundancies in the dataset. Sequences with a p value of less than 1×10⁻¹¹¹ in relation to a database sequence of human origin were specifically excluded. The final result provided the 2396 sequences listed as SEQ ID NOS:1-2396 in the accompanying Sequence Listing and summarized in Table 1 (inserted prior to claims). Each identified polynucleotide represents sequence from at least a partial mRNA transcript.

Table 1 provides: 1) the SEQ ID NO assigned to each sequence for use in the present specification; 2) the cluster to which the sequence is assigned; 3) the sequence name used as an internal identifier of the sequence; 4) the orientation of the insert in the clone (F=forward; R=reverse); 5) the name assigned to the clone from which the sequence was isolated; and 6) the library from which the sequence was originally isolated. Because the provided polynucleotides represent partial mRNA transcripts, two or more polynucleotides of the invention may represent different regions of the same mRNA transcript and the same gene. Thus, if two or more SEQ ID NOS: are identified as belonging to the same clone, then either sequence can be used to obtain the full-length mRNA or gene.

### Example 2

### Results of Public Database Search to Identify Function of Gene Products

SEQ ID NOS:1-2396 were translated in all three reading frames, and the nucleotide sequences and translated amino acid sequences used as query sequences to search for homologous sequences in either the GenBank (nucleotide sequences) or Non-Redundant Protein (amino acid sequences) databases. Query and individual sequences were aligned using the BLAST 2.0 programs (National Center for Biotechnology Information, Bethesda, Md.; see also Altschul, et al. Nucleic Acids Res. (1997) 25:3389-3402). The sequences were masked to various extents to prevent searching of repetitive sequences or poly-A sequences, using the XBLAST program for masking low complexity as described above in Example 1.

Tables 2A and 2B (inserted before the claims) provide the alignment summaries having a p value of 1×10⁻² or less indicating substantial homology between the sequences of the present invention and those of the indicated public databases. Table 2A provides the SEQ ID NO of the query sequence, the accession number of the GenBank database entry of the homologous sequence, and the p value of the alignment. Table 2B provides the SEQ ID NO of the query sequence, the t& accession number of the Non-Redundant Protein database entry of the homologous sequence, and the p value of the alignment. The alignments provided in Tables 2A and 2B are the best available alignment to a DNA or amino acid sequence at a time just prior to filing of the present specification. The activity of the polypeptide encoded by the SEQ ID NOS listed in Tables 2A and 2B can be extrapolated to be substantially the same or substantially similar to the activity of the reported nearest neighbor or closely related sequence. The accession number of the nearest neighbor is reported, providing a publicly available reference to the activities and functions exhibited by the nearest neighbor. The public information regarding the activities and functions of each of the nearest neighbor sequences is incorporated by reference in this application. Also incorporated by reference is all publicly available information regarding the sequence, as well as the putative and actual activities and functions of the nearest neighbor sequences listed in Table 2B and their related sequences. The search program and database used for the alignment, as well as the calculation of the p value are also indicated.

Full length sequences or fragments of the polynucleotide sequences of the nearest neighbors can be used as probes and primers to identify and isolate the full length sequence of the corresponding polynucleotide. The nearest neighbors can indicate a tissue or cell type to be used to construct a library for the full-length sequences of the corresponding polynucleotides.

### Example 3

### Members of Protein Families

SEQ ID NOS:1-2396 were used to conduct a profile search as described in the specification above. Several of the polynucleotides of the invention were found to encode polypeptides having characteristics of a polypeptide belonging to a known protein family (and thus represent members of these protein families) and/or comprising a known functional domain. Table provides the SEQ ID NO: of the query sequence, the profile name, and a brief description of the profile hit.

TABLE 3

| SEQ ID | Profilename | Description |
|---|---|---|
| 410 | ATPases | ATPases Associated with Various Cellular |
| | | Activities |
| 537 | ATPases | ATPases Associated with Various Cellular |
| | | Activities |
| 539 | ATPases | ATPases Associated with Various Cellular |
| | | Activities |
| 540 | ATPases | ATPases Associated with Various Cellular |
| | | Activities |
| 662 | rrm | RNA recognition motif. (aka RRM, RBD, |
| | | or RNP domain) |
| 683 | rrm | RNA recognition motif. (aka RRM, RBD, |
| | | or RNP domain) |
| 707 | dualspecphosphatase | Dual specificity phosphatase, catalytic |
| | | domain |
| 708 | rrm | RNA recognition motif. (aka RRM, RBD, |
| | | or RNP domain) |
| 719 | EFhand | EF-hand |
| 738 | ATPases | ATPases Associated with Various Cellular |
| | | Activities |
| 779 | Zincfing_C2H2 | Zinc finger, C2H2 type |
| 781 | rrm | RNA recognition motif. (aka RRM, RBD, |
| | | or RNP domain) |
| 783 | rrm | RNA recognition motif. (aka RRM, RBD, |
| | | or RNP domain) |
| 1110 | WD_domain | WD domain, G-beta repeats |
| 1415 | Dead_box_helic | DEAD and DEAH box helicases |
| 1533 | C2 | C2 domain (prot. kinase C like) |
| 1633 | dualspecphosphatase | Dual specificity phosphatase, catalytic |
| | | domain |
| 1637 | Dead_box_helic | DEAD and DEAH box helicases |
| 1638 | Dead_box_helic | DEAD and DEAH box helicases |
| 1744 | WD_domain | WD domain, G-beta repeats |
| 1759 | BZIP | Basic region plus leucine zipper |
| | | transcription factors |
| 1993 | WD_domain | WD domain, G-beta repeats |
| 2083 | WD_domain | WD domain, G-beta repeats |
| 2209 | ATPases | ATPases Associated with Various Cellular |
| | | Activities |
| 2228 | ras | Ras family |
| 2287 | ras | Ras family |
| 2300 | neur_chan | Neurotransmitter-gated ion-channel |
| 2302 | tor_domain2 | kinase domain of tors (Christoph |
| | | Reinhard) |
| 2306 | homeobox | Homeobox Domain |
| 2318 | Metallothion | Metallothioneins |
| 2327 | asp | Eukaryotic aspartyl proteases |

Some polynucleotides exhibited multiple profile hits where the query sequence contains overlapping profile regions, and/or where the sequence contains two different functional domains.

Each of the profile hits of Table 3 are described in more detail below. The acronyms for the profiles (provided in parentheses) are those used to identify the profile in the Pfam and Prosite databases. The Pfam database can be accessed through web sites supported by the Washington University, St. Louis (Mo.), The Sanger Centre (United Kingdom); and The Karolinska Institute Center for Genomics Research. The Prosite database is publicaly available through the ExPASy Molecular Biology Server. The public information available on the Pfam and Prosite databases regarding the various profiles, including but not limited to the activities, function, and consensus sequences of various proteins families and protein domains, is incorporated herein by reference.

Eukaryotic Aspartyl Proteases (asp: Pfam Accession No. PF00026).

SEQ ID NO:2327 corresponds to a gene encoding a novel eukaryotic aspartyl protease. Aspartyl proteases, known as acid proteases, (EC 3.4.23.-) are a widely distributed family of proteolytic enzymes (Foltmann B., Essays Biochem. (1981) 17:52; Davies D. R., Annu. Rev. Biophys. Chem. (1990) 19:189; Rao J. K. M., et al., Biochemistry (1991) 30:4663) known to exist in vertebrates, fungi, plants, retroviruses and some plant viruses. Aspartate proteases of eukaryotes are monomeric enzymes which consist of two domains. Each domain contains an active site centered on a catalytic aspartyl residue. The consensus pattern to identify eukaryotic aspartyl protease is: [LIVMFGAC]-[LWMTADN]-[LIVFSA]-D-[ST]-G-[STAV]-[STAPDENQ]-x-[LIVMFSTNC]-x-[LIVMFGTA], where D is the active site residue.

ATPases Associated with Various Cellular Activities (ATPases; Pfam Accession No. PF0004).

SEQ ID NOS:410, 537, 539, 540, 738, and 2209 correspond to a sequence that encodes a member of a family of ATPases Associated with diverse cellular Activities (AAA). The AAA protein family is composed of a large number of ATPases that share a conserved region of about 220 amino acids containing an ATP-binding site (Froehlich et al., J. Cell. Biol. (1991) 114:443; Erdmann et al. Cell (1991) 64:499; Peters et al., EMBO J. (1990) 9:1757; Kunau et al., Biochimie (1993) 75:209-224; Confalonieri et al., BioEssays (1995) 17:639; see also the AAA Server Homepage). The AAA domain, which can be present in one or two copies, acts as an ATP-dependent protein clamp (Confalonieri et al. (1995) BioEssays 17:639) and contains a highly conserved region located in the central part of the domain. The consensus pattern is: [LIVMT]-x-[LIVMT]-[LIVMFI-x-[GATMC]-[ST]-[NS]-x(4)-[LIVM]- D-x-A-[LIFA]-x-R.

Basic Region Plus Leucine Zipper Transcription Factors (BZIP; Pfam Accession No. PF00170).

SEQ ID NO: 1759 represents a polynucleotide encoding a novel member of the family of basic region plus leucine zipper transcription factors. The bZIP superfamily (Hurst, Protein Prof. (1995) 2:105; and Ellenberger, Curr. Opin. Struct. Biol. (1994) 4:12) of eukaryotic DNA-binding transcription factors encompasses proteins that contain a basic region mediating sequence-specific DNA-binding followed by a leucine zipper required for dimerization. The consensus pattern for this protein family is: [KR]-x(1,3)-[RKSAQ]-N-x(2)-[SAQ](2)-x-[RKTAENQ]-x-R-x-[RK].

C2 Domain (C2; Pfam Accession No. PF00168).

SEQ ID NO:1533 corresponds to a sequence encoding a C2 domain, which is involved in calcium-dependent phospholipid binding (Davletov J. Biol. Chem. (1993)268:26386-26390) or, in proteins that do not bind calcium, the domain may facilitate binding to inositol-1,3,4,5-tetraphosphate (Fukuda et al. J. Biol. Chem. (1994) 269:29206-29211; Sutton et al. Cell (1995) 80:929-938). The consensus sequence is: [ACG]-x(2)-L-x(2,3)-D-x(1,2)-[NGSTLIF]-[GTMR]-x-[STAP]-D- [PA]-[FY].

DEAD and DEAH Box Families ATP-Dependent Helicases (Dead Box Helic; Pfam Accession No. PF00270).

SEQ ID NOS:1415, 1637, and 1638 represent polynucleotides encoding a novel member of the DEAD and DEAH box families (Schmid et al., Mol. Microbiol. (1992) 6:283; Linder et al., Nature (1989) 337:121; Wassarman, et al., Nature (1991) 349:463). All members of these families are involved in ATP-dependent, nucleic-acid unwinding. All DEAD box family members share a number of conserved sequence motifs, some of which are specific to the DEAD family, with others shared by other ATP-binding proteins or by proteins belonging to the helicases 'superfamily′ (Hodgman Nature (1988) 333:22 and Nature (1988) 333:578 (Errata)). One of these motifs, called the ‘D-E-A-D-box’, represents a special version of the B motif of ATP-binding proteins. Proteins that have His instead of the second Asp and are ‘D-E-A-H-box’ proteins (Wassarman et al., Nature (1991) 349:463; Harosh, et al., Nucleic Acids Res. (1991) ]9:6331; Koonin, et al., J. Gen. Virol. (1992) 73:989). The following signature patterns are used to identify member for both subfamilies: 1) [LIVMF](2)-D-E-A-D-[RKEN]-x-[LIVMFYGSTN]; and 2) [GSAH]-x-[LIVMF](3)-D-E-[ALIV]-H-[NECR].

Dual Specificity Phosohatase (DSPc; Pfam Accession No. PF00782).

SEQ ID NOS:707 and 1633 correspond to sequences that encode members of a family of dual specificity phosphatases (DSPs). DSPs are Ser/Thr and Tyr protein phosphatases that comprise a tertiary fold highly similar to that of tyrosine-specific phosphatases, except for a “recognition” region connecting helix alphal to strand beta1. This tertiary fold may determine differences in substrate specific between VH-1related dual specificity phosphatase (VHR), the protein tyrosine phosphatases (PTPs), and other DSPs. Phosphatases are important in the control of cell growth, proliferation, differentiation and transformation.

EF Hand (Efhand; Pfam Accession No. PF00036).

SEQ ID NO:719 corresponds to a polynucleotide encoding a member of the EF-hand protein family, a calcium binding domain shared by many calcium-binding proteins belonging to the same evolutionary family (Kawasaki et al., Protein. Prof. (1995) 2:305-490). The domain is a twelve residue loop flanked on both sides by a twelve residue alpha-helical domain, with a calcium ion coordinated in a pentagonal bipyramidal configuration. The six residues involved in the binding are in positions 1, 3, 5, 7, 9 and 12; these residues are denoted by X, Y, Z, -Y, -X and -Z. The invariant Glu or Asp at position 12 provides two oxygens for liganding Ca (bidentate ligand). The consensus pattern includes the complete EF-hand loop as well as the first residue which follows the loop and which seem to always be hydrophobic: D-x-[DNS]-{ILVFYW}-[DENSTG]-[DNQGHRK]-{GP}-[LIVMC]-[DENQSTAGC]-x(2)-[DE]-[LIVMFYW].

Homeobox Domain (homeobox: Pfam Accession No. PF00046).

SEQ ID NO:2306represents a polynucleotide encoding a protein having a homeobox domain. The ‘homeobox’ is a protein domain of 60 amino acids (Gehring In: Guidebook to the Homebox Genes, Duboule D., Ed., ppl-10, Oxford University Press, Oxford, (1994); Buerglin In: Guidebook to the Homebox Genes, pp25-72, Oxford University Press, Oxford, (1994); Gehring Trends Biochem. Sci. (1992) 17:277-280; Gehring et alAnnu. Rev. Genet. (1986) 20:147-173; Schofield Trends Neurosci. (1987) 10:3-6) first identified in number of Drosophila homeotic and segmentation proteins. It is extremely well conserved in many other animals, including vertebrates. This domain binds DNA through a helix-turn-helix type of structure. Several proteins that contain a homeobox domain play an important role in development. Most of these proteins are sequence-specific DNA-binding transcription factors. The homeobox domain is also very similar to a region of the yeast mating type proteins. These are sequence-specific DNA-binding proteins that act as master switches in yeast differentiation by controlling gene expression in a cell type-specific fashion.

A schematic representation of the homeobox domain is shown below. The helix-tum-helix region is shown by the symbols ‘H’ (for helix), and ‘t’ (for turn).

xxxxxxxxxxxxxxxxxxxxxxxxxxxxxxHHHHHHHHtttHHHHHHHHHxxxxxxxxxx
1 60

The pattern detects homeobox sequences 24 residues long and spans positions 34 to 57 of the homeobox domain. The consensus pattern is as follows: [LIVMEYG]-[ASLVR]-x(2)-[LIVMSTACN]-x-[LIVM]-x(4)-[LIV]-[RKNQESTAIY]-[LIVFSTNKH]-W-[FYVC]-x-[NDQTAH]-x(5)-[RKNAIMW].

Metallothioneins (Metalthio; Pfam Accession No. PFOO 131).

SEQ ID NO:2318 corresponds to a polynucleotide encoding a member of the metallothionein (MT) protein family (Hamer Annu. Rev. Biochem. (1986) 55:913-951; and Kagi et al. Biochemistry (1988) 27:8509-8515), small proteins which bind heavy metals such as zinc, copper, cadmium, nickel, etc., through clusters of thiolate bonds. MT's occur throughout the animal kingdom and are also found in higher plants, fungi and some prokaryotes. On the basis of structural relationships MT's have been subdivided into three classes. Class I includes mammalian MT's as well as MT's from crustacean and molluscs, but with clearly related primary structure. Class II groups together MT's from various species such as sea urchins, fungi, insects and cyanobacteria which display none or only very distant correspondence to class I MT's. Class III MT's are atypical polypeptides containing gamma-glutamylcysteinyl units. The consensus pattern for this protein family is: C-x-C-[GSTAP]-x(2)-C-x-C-x(2)-C-x-C-x(2)-C-x-K.

Neurotransmitter-Gated Ion-Channel (neur chan; Pfam Accession No. PF00065).

SEQ ID NO:2300 corresponds to a sequence encoding a neurotransmitter-gated ion channel. Neurotransmitter-gated ion-channels, which provide the molecular basis for rapid signal transmission at chemical synapses, are post-synaptic oligomeric transmembrane complexes that transiently form a ionic channel upon the binding of a specific neurotransmitter. Five types of neurotransmitter-gated receptors are known: 1) nicotinic acetylcholine receptor (AchR); 2) glycine receptor; 3) gamma-aminobutyric-acid (GABA) receptor; 4) serotonin 5HT3 receptor; and 5) glutamate receptor. All known sequences of subunits from neurotransmitter-gated ion-channels are structurally related, and are composed of a large extracellular glycosylated N-terminal ligand-binding domain, followed by three hydrophobic transmembrane regions that form the ionic channel, followed by an intracellular region of variable length. A fourth hydrophobic region is found at the C-terminal of the sequence. The consensus pattern is: C-x-[LIVMFQ]-x-[L MF]-x(2)-[FY]-P-x-D-x(3)-C, where the two C's are linked by a disulfide bond.

Ras Family Proteins (ras: Pfam Accession No. PF00071).

SEQ ID NOS:2228 and 2287 represent polynucleotides encoding the ras family of small GTP/GDP-binding proteins (Valencia et al., 1991, Biochemistry 30:4637-4648). Ras family members generally require a specific guanine nucleotide exchange factor (GEF) and a specific GTPase activating protein (GAP) as stimulators of overall GTPase activity. Among ras-related proteins, the highest degree of sequence conservation is found in four regions that are directly involved in guanine nucleotide binding. The first two constitute most of the phosphate and Mg2+ binding site (PM site) and are located in the first half of the G-domain. The other two regions are involved in guanosine binding and are located in the C-terminal half of the molecule. Motifs and conserved structural features of the ras-related proteins are described in Valencia et al., 1991, Biochemistry 30:46374648. A major consensus pattern of ras proteins is: D-T-A-G-Q-E-K-[LF]-G-G-L-R-[DE]-G-Y-Y.

RNA Recognition Motif (rrm; Pfam Accession No. PF00076).

SEQ ID NOS:662, 683, 708, 781, and 783 correspond to sequence encoding an RNA recognition motif, also known as an RRM, RBD, or RNP domain. This domain, which is about 90 amino acids long, is contained in eukaryotic proteins that bind single-stranded RNA (Bandziulis et al. Genes Dev. (1989) 3:431437; Dreyfuss et al. Trends Biochem. Sci. (1988) 13:86-91). Two regions within the RNA-binding domain are highly conserved: the first is a hydrophobic segment of six residues (which is called the RNP-2 motif), the second is an octapeptide motif (which is called RNP-1 or RNP-CS). The consensus pattern is: [RK]-G-{EDRKHPCG}-[AGSCI]-[FY]-[LIVA]-x-[FYLM].

Kinase Domain of Tors (tor domain2).

SEQ ID NO:2302 corresponds to a member of the TOR lipid kinase protein family. This family is composed of large proteins with a lipid and protein kinase domain and characterized through their sensitivity to rapamycin (an antifungal compound). TOR proteins are involved in signal transduction downstream of P13 kinase and many other signals. TOR (also called FRAP, RAFT) plays a role in regulating protein synthesis and cell growth., and in yeast controls translation initiation and early GI progression. See, e.g., Barbet et al. Mol Biol Cell. (1996) 7(1):25-42; Helliwell et al. Genetics (1998) 148:99-112.

WD Domain, G-Beta Repeats (WD domain; Pfam Accession No. PF00400).

SEQ ID NOS:1110, 1744, 1993, and 2083 represent novel members of the WD domain/G-beta repeat family. Beta-transducin (G-beta) is one of the three subunits (alpha, beta, and gamma) of the guanine -nucleotide-binding proteins (G proteins) which act as intermediaries in the transduction of signals generated by transmembrane receptors (Gilman, Annu. Rev. Biochem. (1987) 56:615). The alpha subunit binds to and hydrolyzes GTP; the functions of the beta and gamma subunits are less clear but they seem to be required for the replacement of GDP by GTP as well as for membrane anchoring and receptor recognition. In higher eukaryotes, G-beta exists as a small multigene family of highly conserved proteins of about 340 amino acid residues. Structurally, G-beta consists of eight tandem repeats of about 40 residues, each containing a central Trp-Asp motif (this type of repeat is sometimes called a WD40 repeat). The consensus pattern for the WD domain/G-Beta repeat family -is: [LIVMSTAC]-[LIVMFYWSTAGC]-[LIMSTAG]-[LIVMSTAGC]-x(2)-[DN]-x(2)-[LIVMWSTAC]-x-[LIVMFSTAG]-W-[DEN]-[LIVMFSTAGCN].

Zinc Finger, C2H2 Type (Zincefing C2H2: Pfam Accession No. PF00096).

SEQ ID NO:779 corresponds to a polynucleotid encoding a member of the C2H2 type zinc finger protein family, which contain zinc finger domains that facilitate nucleic acid binding (Klug et al., Trends Biochem. Sci. (1987) 12:464; Evans et al., Cell (1988) 52: 1; Payre et al., FEBS Lett. (1988) 234:245; Miller et al., EMBO J. (1985) 4:1609; and Berg, Proc. Natl. Acad. Sci. USA (1988) 85:99).

In addition to the conserved zinc ligand residues, a number of other positions are also important for the structural integrity of the C2H2 zinc fingers. (Rosenfeld et al., J. Biomol. Struct. Dyn. (1993) 11:557) The best conserved position, which is generally an aromatic or aliphatic residue, is located four residues after the second cysteine. The consensus pattern for C2H2 zinc fingers is: C-x(2,4)-C-x(3)-[LIVMFYWC]-x(8)-H-x(3,5)-H. The two C's and two H's are zinc ligands.

### Example 4

### Differential Expression of Polvnucleotides of the Invention: Description of Libraries and Detection of Differential Expression

The relative expression levels of the polynucleotides of the invention was assessed in several libraries prepared from various sources, including cell lines and patient tissue samples. Table 4 provides a summary of these libraries, including the shortened library name (used hereafter), the mPNA source used to prepared the cDNA library, and the approximate number of clones in the library.

TABLE 4
Description of cDNA Libraries

| | | Number of |
|---|---|---|
| Library | | Clones in |
| (Lib#) | Description | Library |
| 1 | Human Colon Cell Line Km12 L4: High Metastatic | 308731 |
| | Potential (derived from Km12C) | |
| 2 | Human Colon Cell Line Km12C: Low Metastatic | 284771 |
| | Potential | |
| 3 | Human Breast Cancer Cell Line MDA-MB-231: | 326937 |
| | High Metastatic Potential; micro-mets in lung | |
| 4 | Human Breast Cancer Cell Line MCF7: Non | 318979 |
| | Metastatic | |
| 8 | Human Lung Cancer Cell Line MV-522: High | 223620 |
| | Metastatic Potential | |
| 9 | Human Lung Cancer Cell Line UCP-3: Low | 312503 |
| | Metastatic Potential | |
| 12 | Human microvascular endothelial cells (HMVEC)— | 41938 |
| | UNTREATED (PCR (OligodT) cDNA library) | |
| 13 | Human microvascular endothelial cells (HMVEC)— | 42100 |
| | bFGF TREATED (PCR (OligodT) cDNA library) | |
| 14 | Human microvascular endothelial cells (HMVEC)— | 42825 |
| | VEGF TREATED (PCR (OligodT) cDNA library) | |
| 15 | Normal Colon—UC#2 Patient (MICRO- | 282722 |
| | DISSECTED PCR (OligodT) cDNA library) | |
| 16 | Colon Tumor—UC#2 Patient (MICRO- | 298831 |
| | DISSECTED PCR (OligodT) cDNA library) | |
| 17 | Liver Metastasis from Colon Tumor of UC#2 | 303467 |
| | Patient (MICRODISSECTED PCR (OligodT) | |
| | cDNA library) | |
| 18 | Normal Colon—UC#3 Patient (MICRO- | 36216 |
| | DISSECTED PCR (OligodT) cDNA library) | |
| 19 | Colon Tumor—UC#3 Patient (MICRO- | 41388 |
| | DISSECTED PCR (OligodT) cDNA library) | |
| 20 | Liver Metastasis from Colon Tumor of UC#3 | 30956 |
| | Patient (MICRODISSECTED PCR (OligodT) | |
| | cDNA library) | |
| 21 | GRRpz Cells derived from normal prostate | 164801 |
| | epithelium | |
| 22 | WOca Cells derived from Gleason Grade 4 prostate | 162088 |
| | cancer epithelium | |
| 23 | Normal Lung Epithelium of Patient #1006 | 306198 |
| | (MICRODISSECTED PCR (OligodT) cDNA | |
| | library) | |
| 24 | Primary tumor, Large Cell Carcinoma of Patient | 309349 |
| | #1006 (MICRODISSECTED PCR (OligodT) cDNA | |
| | library) | |

The KM12L4 cell line (Morikawa, et al., Cancer Research (1988) 48:6863) is derived from the KM12C cell line (Morikawa et al. Cancer Res. (1988) 48:1943-1948),. The KM12C cell line, which is poorly metastatic (low metastatic) was established in culture from a Dukes' stage B₂ surgical specimen (Morikawa et al. Cancer Res. (1988) 48:6863). The KM12L4-A is a highly metastatic subline derived from KM12C (Yeatman et al. Nucl. Acids. Res. (1995) 23:4007; Bao-Ling et al. Proc. Annu. Meet. Am. Assoc. Cancer. Res. (1995) 21:3269). The KM12C and KM12C-derived cell lines (e.g., KM12L4, KMI2L4-A, etc.) are well-recognized in the art as a model cell line for the study of colon cancer (see, e.g., Moriakawa et al., supra; Radinsky et al. Clin. Cancer Res. (1995) 1:19; Yeatman et al., (1995) supra; Yeatman et al. Clin. Exp. Metastasis (1996) 14:246). The MDA-MB-231 cell line was originally isolated from pleural effusions (Cailleau, J. Natl. Cancer. Inst. (1974) 53:661), is of high metastatic potential, and forms poorly differentiated adenocarcinoma grade II in nude mice consistent with breast carcinoma. The MCF7 cell line was derived from a pleural effusion of a breast adenocarcinoma and is non-metastatic. The MDA-MB-231 and MCF-7cell lines are well-recognized in the art as a models for the study of human breast -cancer (see, e.g., Chandrasekaran et al., Cancer Res. (1979) 39:870; Gastpar et al., J Med Chem (1998) 41:4965; Ranson et al., Br J Cancer (1998) 77:1586; and Kuang et al., Nucleic Acids Res (1998) 26:1116).

The MV-522 cell line is derived from a human lung carcinoma and is of high metastatic potential. The UCP-3 cell line is a low metastatic human lung carcinoma cell line; the MV-522 is a high metastatic variant of UCP-3. These cell lines are well-recognized in the art as models for the study of human lung cancer (see, e.g., Varki et al., Int J Cancer (1987) 40:46 (UCP-3); Varki et al., Tumour Biol. (1990) 11:327; (MV-522 and UCP-3); Varki et al., Anticancer Res. (1990) 10:637; (MV-522); Kelner et al., Anticancer Res (1995) 15:867 (MV-522); and Zhang et al., Anticancer Drugs (1997) 8:696 (MV522)). The samples of libraries 15-20 are derived from two different patients (UC#2, and UC#3). The bFGF-treated HMVEC were prepared by incubation with bFGF at 10 ng/ml for 2 hrs; the VEGF-treated HMVEC were prepared by incubation with 20ng/ml VEGF for 2 hrs. Following incubation with the respective growth factor, the cells were washed and lysis buffer added for RNA preparation. The GRRpz and WOca cell lines were provided by Dr. Donna M. Peehl, Department of Medicine, Stanford University School of Medicine. GRRpz was derived from normal prostate epithelium. The WOca cell line is a Gleason Grade 4 cell line.

Each of the libraries is composed of a collection of cDNA clones that in turn are representative of the mRNAs expressed in the indicated mRNA source. In order to facilitate the analysis of the millions of sequences in each library, the sequences were assigned to clusters. The concept of “cluster of clones” is derived from a sorting/grouping of cDNA clones based on their hybridization pattern to a panel of roughly 300 7bp oligonucleotide probes (see Drmanac et al., Genomics (1996) 37(1):29). Random cDNA clones from a tissue library are hybridized at moderate stringency to 300 7bp oligonucleotides. Each oligonucleotide has some measure of specific hybridization to that specific clone. The combination of 300 of these measures of hybridization for 300 probes equals the “hybridization signature” for a specific clone. Clones with similar sequence will have similar hybridization signatures. By developing a sorting/grouping algorithm to analyze these signatures, groups of clones in a library can be identified and brought together computationally. These groups of clones are termed “clusters”. Depending on the stringency of the selection in the algorithm (similar to the stringency of hybridization in a classic library cDNA screening protocol), the “purity” of each cluster can be controlled. For example, artifacts of clustering may occur in computational clustering just as artifacts can occur in “wet-lab” screening of a cDNA library with 400 bp cDNA fragments, at even the highest stringency. The stringency used in the implementation of cluster herein provides groups of clones that are in general from the same cDNA or closely related cDNAs. Closely related clones can be a result of different length clones of the same cDNA, closely related clones from highly related gene families, or splice variants of the same cDNA.

Differential expression for a selected cluster was assessed by first determining the number of cDNA clones corresponding to the selected cluster in the first library (Clones in 1^{st}), and the determining the number of cDNA clones corresponding to the selected cluster in the second library (Clones in 2^{nd}). Differential expression of the selected cluster in the first library relative to the - second library is expressed as a “ratio” of percent expression between the two libraries. In general, the “ratio” is calculated by: 1) calculating the percent expression of the selected cluster in the first library by dividing the number of clones corresponding to a selected cluster in the first library by the total number of clones analyzed from the first library; 2) calculating the percent expression of the selected cluster in the second library by dividing the number of clones corresponding to a selected cluster in a second library by the total number of clones analyzed from the second library; 3) dividing the calculated percent expression from the first library by the calculated percent expression from the second library. If the “number of clones” corresponding to a selected cluster in a library is zero, the value is set at 1 to aid in calculation. The formula used in calculating the ratio takes into account the “depth” of each of the libraries being compared, i.e., the total number of clones analyzed in each library.

In general, a polynucleotide is said to be significantly differentially expressed between two samples when the ratio value is greater than at least about 2, preferably greater than at least about 3, more preferably greater than at least about 5, where the ratio value is calculated using the method described above. The significance of differential expression is determined using a z score test (Zar, Biostatistical Analysis, Prentice Hall, Inc., USA, “Differences between Proportions,” pp 296-298 (1974).

### Example 5

### Differential Expression of Genes Corresponding to Polynucleotides of the Invention

A number of polynucleotide sequences have been identified that are differentially expressed between, for example, cells derived from high metastatic potential cancer tissue and low metastatic cancer cells, and between cells derived from metastatic cancer tissue and normal tissue. Evaluation of the levels of expression of the genes corresponding to these sequences can be valuable in diagnosis, prognosis, and/or treatment (e.g., to facilitate rationale design of therapy, monitoring during and after therapy, etc.). Moreover, the genes corresponding to differentially expressed sequences described herein can be therapeutic targets due to their involvement in regulation (e.g., inhibition or promotion) of development of, for example, the metastatic phenotype. For example, sequences that correspond to genes that are increased in expression in high metastatic potential cells relative to normal or non-metastatic tumor cells may encode genes or regulatory sequences involved in processes such as angiogenesis, differentiation, cell replication, and metastasis.

Detection of the relative expression levels of differentially expressed polynucleotides described herein can provide valuable information to guide the clinician in the choice of therapy. For example, a patient sample exhibiting an expression level of one or more of these polynucleotides that corresponds to a gene that is increased in expression in metastatic or high metastatic potential cells may warrant more aggressive treatment for the patient. In contrast, detection of expression levels of a polynucleotide sequence that corresponds to expression levels associated with that of low metastatic potential cells may warrant a more positive prognosis than the gross pathology would suggest.

The differential expression of the polynucleotides described herein can thus be used as, for example, diagnostic markers, prognostic markers, for risk assessment, patient treatment and the like.

These polynucleotide sequences can also be used in combination with other known molecular and/or biochemical markers. The following examples provide relative expression levels of polynucleotides from specified cell lines and patient tissue samples.

The differential expression data for polynucleotides of the invention that have been identified as being differentially expressed across various combinations of the libraries described above is summarized in Table 5 (inserted prior to the claims). Table 5 provides: 1) the Sequence Identification Number (“SEQ”) assigned to the polynucleotide; 2) the cluster (“CLST”) to which the polynucleotide has been assigned as described above; 3) the library comparisons that resulted in identifcation of the polynucleotide as being differentially expressed (“Library Pair A,B”), with shorthand names of the compared libraries provided in parentheses following the library numbers;, 4) the number of clones corresponding to the polynucleotide in the first library listed (“A”); 5) the number of clones corresponding to the polynucleotide in the second library listed (“B”); 6) the “A/B” where the comparison resulted in a finding that the number of clones in library A is greater than the number of clones in library B; and 7) the “B/A” where the comparison resulted in a finding that the number of clones in library B is greater than the number of clones in library A.

### Example 6

### Source of Biological Materials for Microarray-Based Experiments

The biological materials used in the experiments described in the subsequent examples relating to microarry data are described below.

Source of Patient Tissue Samples

Normal and cancerous tissues were collected from patients using laser capture microdissection (LCM) techniques, which techniques are well known in the art (see, e.g., Ohyama et al. (2000) Biotechniques 29:530-6; Curran et al. (2000) Mol. Pathol. 53:64-8; Suarez-Quian et al. (1999) Biotechniques 26:328-35; Simone et al. (1998) Trends Genet 14:272-6; Conia et al. (1997) J. Clin. Lab. Anal. 11:28-38; Emmert-Buck et al. (1996) Science 274:998-1001). Table 9 (inserted following the last page of the Examples) provides information about each patient from which the samples were isolated, including: the Patient ID and Path ReportID, numbers assigned to the patient and the pathology reports for identification purposes; the anatomical location of the tumor (AnatomicalLoc); The Primary Tumor Size; the Primary Tumor Grade; the Histopathologic Grade; a description of local sites to which the tumor had invaded (Local Invasion); the presence of lymph node metastases (Lymph Node Metastasis); incidence of lymph node metastases (provided as number of lymph nodes positive for metastasis over the number of lymph nodes examined) (Incidence Lymphnode Metastasis); the Regional Lymphnode Grade; the identification or detection of metastases to sites distant to the tumor and their location (Distant Met & Loc);a description of the distant metastases (Description Distant Met); the grade of distant metastasis (Distant Met Grade); and general comments about the patient or the tumor (Comments). Adenoma was not described in any of the patients. ; adenoma dysplasia (described as hyperplasia by the pathologist) was described in Patient ID No. 695. Extranodal extensions were described in two patients, Patient ID Nos. 784 and 791. Lymphovascular invasion was described in seven patients, Patient ID Nos. 128, 278, 517, 534, 784, 786, and 791. Crohn's-like infiltrates were described in seven patients, Patient ID Nos. 52, 264, 268, 392, 393, 784, and 791.

Polynucleotides on Arrays

Polynucleotides spotted on the arrays were generated by PCR amplification of clones derived from cDNA libraries. The clones used for amplification were either the clones from which the sequences described herein (SEQ ID NOS:1-2396) were derived, or are clones having inserts with significant polynucleotide sequence overlap wih the sequences described herein (SEQ ID NO: 1-2396) as determined by BLAST2 homology searching.

### Example 7

### Microarray Design

Each array used in the examples below had an identical spatial layout and control spot set. Each microarray was divided into two areas, each area having an array with, on each half, twelve groupings of 32×12 spots for a total of about 9,216 spots on each array. The two areas are spotted identically which provide for at least two duplicates of each clone per array. Spotting was accomplished using PCR amplified products from 0.5kb to 2.0 kb and spotted using a Molecular Dynamics Gen III spotter according to the manufacturer's recommendations. The first row of each of the 24 regions on the array had about 32 control spots, including 4 negative control spots and 8 test polynucleotides.

The test polynucleotides were spiked into each sample before the labeling reaction with a range of concentrations from 2-600 pg/slide and ratios of 1:1 . For each array design, two slides were hybridized with the test samples reverse-labeled in the labeling reaction.

This provided for about 4 duplicate measurements for each clone, two of one color and two of the other, for each sample.

### Example 8

### Identification Of Differentially Expressed Genes

cDNA probes were prepared from total RNA isolated from the patient cells described in Example 6. Since LCM provides for the isolation of specific cell types to provide a substantially homogenous cell sample, this provided for a similarly pure RNA sample.

Total RNA was first reverse transcribed into cDNA using a primer containing a T7 RNA polymerase promoter, followed by second strand DNA synthesis. cDNA was then transcribed in vitro to produce antisense RNA using the T7 promoter-mediated expression (see, e.g., Luo et al. (1999) Nature Med 5:117-122), and the antisense RNA was then converted into cDNA. The second set of cDNAs were again transcribed in vitro, using the T7 promoter, to provide antisense RNA. Optionally, the RNA was again converted into cDNA, allowing for up to a third round of T7-mediated amplification to produce more antisense RNA. Thus the procedure provided for two or three rounds of in? vitro transcription to produce the final RNA used for fluorescent labeling. Fluorescent probes were generated by first adding control RNA to the antisense RNA mix, and producing fluorescently labeled cDNA from the RNA starting material. Fluorescently labeled cDNAs prepared from the tumor RNA sample were compared to fluorescently labeled cDNAs prepared from normal cell RNA sample. For example, the cDNA probes from the normal cells were labeled with Cy3 fluorescent dye (green) and the cDNA probes prepared from the tumor cells were labeled with Cy5 fluorescent dye (red).

The differential expression assay was performed by mixing equal amounts of probes from tumor cells and normal cells of the same patient. The arrays were prehybridized by incubation for about 2 hrs at 60° C. in 5× SSC/0.2% SDS/1 mM EDTA, and then washed three times in water and twice in isopropanol. Following prehybridization of the array, the probe mixture was then hybridized to the array under conditions of high stringency (overnight at 42° C. in 50% formamide, 5X SSC, and 0.2% SDS. After hybridization, the array was washed at 55° C. three times as follows: 1) first wash in 1× SSC/0.2% SDS; 2) second wash in 0.1× SSC/0.2% SDS; and 3) third wash in 0.1× SSC.

The arrays were then scanned for green and red fluorescence using a Molecular Dynamics Generation III dual color laser-scanner/detector. The images were processed using BioDiscovery Autogene software, and the data from each scan set normalized to provide for a ratio of expression relative to normal. Data from the microarray experiments was analyzed according to the algorithms described in U.S. application serial No. 60/252,358, filed Nov. 20,2000, by E. J. Moler, M. A. Boyle, and F. M. Randazzo, and entitled “Precision and accuracy in cDNA microarray data,” which application is specifically incorporated herein by reference.

The experiment was repeated, this time labeling the two probes with the opposite color in order to perform the assay in both “color directions.” Each experiment was sometimes repeated with two more slides (one in each color direction). The level fluorescence for each sequence on the array expressed as a ratio of the geometric mean of 8 replicate spots/genes from the four arrays or 4 replicate spots/gene from 2 arrays or some other permutation. The data were normalized using the spiked positive controls present in each duplicated area, and the precision of this normalization was included in the final determination of the significance of each differential. The fluorescent intensity of each spot was also compared to the negative controls in each duplicated area to determine which spots have detected significant expression levels in each sample.

A statistical analysis of the fluorescent intensities was applied to each set of duplicate spots to assess the precision and significance of each differential measurement, resulting in a p-value testing the null hypothesis that there is no differential in the expression level between the tumor and normal samples of each patient. During initial analysis of the microarrays, the hypothesis was accepted if p>10⁻³, and the differential ratio was set to 1.000 for those spots. All other spots have a significant difference in expression between the tumor and normal sample. If the tumor sample has detectable expression and the normal does not, the ratio is truncated at 1000 since the value for expression in the normal sample would be zero, and the ratio would not be a mathematically useful value (e.g., infinity). If the normal sample has detectable expression and the tumor does not, the ratio is truncated to 0.001, since the value for expression in the tumor sample would be zero and the ratio would not be a mathematically useful value. These latter two situations are referred to herein as “on/off.” Database tables were populated using a 95% confidence level (p>0.05).

Tables 10-14:clf summarizes the results of the differential expression analysis, where the difference in the expression level in the colon tumor cell relative to the matched normal colon cells is greater than or equal to 2 fold (“>=2×”), 2.5 fold (“>=2.5×”), or 5 fold (“>=5×”) in at least 20% or more of the patients analyzed. Each table provides: the SEQ ID NO; the percentage of patients tested having a colon tumor that exhibited at least 2 fold (“>=2×”), 2.5 fold (“>=2.5×”), or 5 fold (“>=5×”) increase in expression levels of the indicated gene relative to matched normal colon tissue; and the ratio data for each patient sample tested (columns headed by “P#,” indicating the Patient Identification Number, e.g., “P 15” indicates the ration data for patient 15).

In general, a polynucleotide is said to represent a significantly differentially expressed gene between two samples when there is detectable levels of expression in at least one sample and the ratio value is greater than at least about 1.2 fold, preferably greater than at least about 1.5 fold, more preferably greater than at least about 2 fold, where the ratio value is calculated using the method described above.

A differential expression ratio of 1 indicates that the expression level of the gene in the tumor cell was not statistically different from expression of that gene in normal colon cells of the same patient. A differential expression ratio significantly greater than I in cancerous colon cells relative to normal colon cells indicates that the gene is increased in expression in cancerous cells relative to normal cells, indicating that the gene plays a role in the development of the cancerous phenotype, and may be involved in promoting metastasis of the cell. Detection of gene products from such genes can provide an indicator that the cell is cancerous, and may provide a therapeutic and/or diagnostic target.

Likewise, a differential expression ratio significantly less than 1 in cancerous colon cells relative to normal colon cells indicates that, for example, the gene is involved in suppression of the cancerous phenotype. Increasing activity of the gene product encoded by such a gene, or replacing such activity, can provide the basis for chemotherapy. Such gene can also serve as markers of cancerous cells, e.g., the absence or decreased presence of the gene product in a colon cell relative to a normal colon cell indicates that the cell may be cancerous.

### Example 9

### Functional Analysis Of Gene Products Differentially Expressed In Cancer In Patients

The gene products of genes differentially expressed in cancerous cells are further analyzed to confirm the role and function of the gene product in tumorgenesis, e.g., in promoting or inhibiting development of a metastatic phenotype.

Blocking Expression of Gene Products Using Antisense

The effect of single genes upon development of cancer is assessed through use of antisense oligonucleotides specific for sequences corresponding to a selected sequence.

Antisense oligonucleotides are prepared based upon a selected sequence that corresponds to a gene of interest. The antisense oligonucleotide is introduced into a test cell and the effect upon expression of the corresponding gene, as well as the effect upon a phenotype of interest assessed (e.g., a normal cell is examined for induction of the cancerous phenotype, or a cancerous cell is examined for suppression of a cancerous phenotype (e.g., suppression of metastasis)).

Blocking Function of Gene Products Using Gene Product-Specific Antibodies and/or Small Molecule Inhibitors

The function of gene products corresponding to genes/clusters identified herein can be assessed by blocking function of the gene products in the cell. For example, where the gene product is secreted, blocking antibodies can generated and added to cells to examine the effect upon the cell phenotype in the context of, for example, the transformation of the cell to a cancerous, particularly a metastatic, phenotype. In order to generate antibodies, a clone corresponding to a selected gene product/cluster is selected, and a sequence that represents a partial or complete coding sequence is obtained. The resulting clone is then expressed, the polypeptide produced isolated, and antibodies generated. The antibodies are then combined with cells and the effect upon tumorigenesis assessed.

Where the gene product of the gene/clusters identified herein exhibits sequence homology to a protein of known function (e.g., to a specific kinase or protease) and/or to a protein family of known function (e.g., contains a domain or other consensus sequence present in a protease family or in a kinase family), then the role of the gene product in tumorigenesis, as well as the activity of the gene product, can be examined using small molecule that inhibit or enhance function of the corresponding protein or protein family.

Those skilled in the art will recognize, or be able to ascertain, using not more than routine experimentation, many equivalents to the specific embodiments of the invention described herein. Such specific embodiments and equivalents are intended to be encompassed by the following claims.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Deposit Information.

The following materials were deposited with the American Type Culture Collection (CMCC=Chiron Master Culture Collection).

TABLE 6
Cell Lines Deposited with ATCC

| | | ATCC | CMCC |
|---|---|---|---|
| Cell Line | Deposit Date | Accession No. | Accession No. |
| KM12L4 | Mar. 19, 1998 | CRL-12496 | 11606 |
| Km12C | May 15, 1998 | CRL-12533 | 11611 |
| MDA-MB-231 | May 15, 1998 | CRL-12532 | 10583 |
| MCF-7 | Oct. 9, 1998 | CRL-12584 | 10377 |

In addition, pools of selected clones, as well as libraries containing specific clones, were assigned an “ES” number (internal reference) and deposited with the ATCC. Table 7 (inserted before the claims) provides the ATCC Accession Nos. and internal references (CMCC Nos.) of the ES deposits, all of which were deposited on or before the filing date of the present application. The names of the clones contained within each of these deposits are provided in Table 8 (inserted before the claims).

The above material has been deposited with the American Type Culture Collection, Rockville, Md., under the accession number indicated. These deposits will be maintained under the terms of the Budapest Treaty on the International Recognition of the Deposit of Microorganisms for the Purposes of Patent Procedure. The deposit will be maintained for a period of at least 30 years following issuance of this patent, or for the enforceable life of the patent, whichever is greater. Upon the granting of a patent, all restrictions on the availability to the public of the deposited material will be irrevocably removed.

The deposits described herein are provided merely as convenience to those of skill in the art, and is not an admission that a deposit is required under 35 U.S.C. §112. The sequence of the polynucleotides contained within the deposited material, as well as the amino acid sequence of the polypeptides encoded thereby, are incorporated herein by reference and are controlling in the event of any conflict with the written description of sequences herein. A license may be required to make, use, or sell the deposited material, and no such license is granted hereby.

Retrieval of Individual Clones from Deposit of Pooled Clones.

Where the ATCC deposit is composed of a pool of cDNA clones or a library of cDNA clones, the deposit was prepared by first transfecting each of the clones into separate bacterial cells. The clones in the pool or library were then deposited as a pool of equal mixtures in the composite deposit. Particular clones can be obtained from the composite deposit using methods well known in the art. For example, a bacterial cell containing a particular clone can be identified by isolating single colonies, and identifying colonies containing the specific clone through standard colony hybridization techniques, using an oligonucleotide probe or probes designed to specifically hybridize to a sequence of the clone insert (e.g., a probe based upon unmasked sequence of the encoded polynucleotide having the indicated SEQ ID NO). The probe should be designed to have a Tₘ of approximately 80° C. (assuming 2° C. for each A or T and 4° C. for each G or C). Positive colonies can then be picked, grown in culture, and the recombinant clone isolated. Alternatively, probes designed in this manner can be used to PCR to isolate a nucleic acid molecule from the pooled clones according to methods well known in the art, e.g., by purifying the cDNA from the deposited culture pool, and using the probes in PCR reactions to produce an amplified product having the corresponding desired polynucleotide sequence.

TABLE 7

| Table 7 | | |
|---|---|---|
| ES # | CMCC# | ATCC Deposit# |
| 85 | 5175 | PTA-1313 |
| 86 | 5176 | PTA-1314 |
| 87 | 5177 | PTA-1315 |
| 88 | 5178 | PTA-1316 |
| 89 | 5179 | PTA-1317 |
| 90 | 5180 | PTA-1318 |
| 91 | 5181 | PTA-1319 |
| 92 | 5182 | PTA-1320 |
| 93 | 5183 | PTA-1321 |
| 94 | 5184 | PTA-1322 |
| 95 | 5185 | PTA-1323 |
| 96 | 5186 | PTA-1324 |
| 97 | 5187 | PTA-1325 |
| 98 | 5188 | PTA-1326 |
| 99 | 5189 | PTA-1327 |
| 100 | 5190 | PTA-1328 |
| 101 | 5191 | PTA-1329 |
| 102 | 5192 | PTA-1330 |
| 103 | 5193 | PTA-1331 |
| 104 | 5194 | PTA-1332 |
| 105 | 5195 | PTA-1333 |
| 106 | 5196 | PTA-1334 |
| 107 | 5197 | PTA-1335 |
| 108 | 5198 | PTA-1336 |
| 109 | 5199 | PTA-1372 |
| 110 | 5200 | PTA-1373 |
| 111 | 5201 | PTA-1374 |
| 112 | 5202 | PTA-1375 |
| 113 | 5203 | PTA-1376 |
| 114 | 5204 | PTA-1377 |
| 115 | 5205 | PTA-1378 |
| 116 | 5206 | PTA-1379 |
| 117 | 5207 | PTA-1380 |
| 118 | 5208 | PTA-1381 |
| 122 | 5212 | PTA-1382 |
| 123 | 5213 | PTA-1383 |
| 124 | 5214 | PTA-1384 |
| 125 | 5215 | PTA-1385 |
| 126 | 5216 | PTA-1386 |
| 127 | 5217 | PTA-1387 |
| 128 | 5218 | PTA-1388 |
| 129 | 5219 | PTA-1389 |
| 130 | 5220 | PTA-1390 |
| 131 | 5221 | PTA-1391 |
| 132 | 5222 | PTA-1392 |
| 133 | 5223 | PTA-1393 |
| 134 | 5209 | PTA-1431 |
| 135 | 5210 | PTA-1432 |
| 136 | 5238 | PTA-1497 |

TABLE 8

| ES No. | Clone Name | ES No. | Clone Name |
|---|---|---|---|
| ES 85 | M00057077B:D02 | ES 109 | M00027658B:G03 |
| ES 85 | M00057078D:C12 | ES 109 | M00027660C:E03 |
| ES 85 | M00057079D:E09 | ES 109 | M00027660C:E03 |
| ES 85 | M00057080C:C02 | ES 109 | M00027665B:D01 |
| ES 85 | M00057085A:A03 | ES 109 | M00027681D:D02 |
| ES 85 | M00057088B:C02 | ES 109 | M00027699D:D02 |
| ES 85 | M00057091A:C03 | ES 109 | M00027717C:G05 |
| ES 85 | M00057091A:C04 | ES 109 | M00027733D:D05 |
| ES 85 | M00057091C:E12 | ES 109 | M00027742C:B01 |
| ES 85 | M00057093B:F09 | ES 109 | M00027742C:B01 |
| ES 85 | M00057099C:C08 | ES 109 | M00027747D:D01 |
| ES 85 | M00057100C:E09 | ES 109 | M00027757A:B06 |
| ES 85 | M00057100D:B03 | ES 109 | M00027781D:E04 |
| ES 85 | M00057103A:E11 | ES 109 | M00027786D:B01 |
| ES 85 | M00057103A:H09 | ES 109 | M00027803A:H10 |
| ES 85 | M00057104B:F08 | ES 109 | M00027806C:H05 |
| ES 85 | M00057106B:A03 | ES 109 | M00027808D:G10 |
| ES 85 | M00057106C:E02 | ES 109 | M00027817B:B11 |
| ES 85 | M00057106D:B06 | ES 109 | M00027820C:C02 |
| ES 85 | M00057108B:F04 | ES 109 | M00027823C:G07 |
| ES 85 | M00057108D:E09 | ES 109 | M00027829C:D02 |
| ES 85 | M00057108D:E09 | ES 109 | M00027833C:D01 |
| ES 85 | M00057112D:B09 | ES 110 | M00042345A:F12 |
| ES 85 | M00057114D:B10 | ES 110 | M00042523A:C05 |
| ES 85 | M00057117D:G11 | ES 110 | M00042523C:E08 |
| ES 85 | M00057118C:C02 | ES 110 | M00042525D:E01 |
| ES 85 | M00057120D:E12 | ES 110 | M00042527B:D07 |
| ES 85 | M00057124B:D10 | ES 110 | M00042528C:F11 |
| ES 85 | M00057127A:F11 | ES 110 | M00042529C:G07 |
| ES 85 | M00057127B:G07 | ES 110 | M00042532A:F08 |
| ES 85 | M00057130C:H11 | ES 110 | M00042534A:B07 |
| ES 85 | M00057131C:B01 | ES 110 | M00042536D:F01 |
| ES 85 | M00057132C:F08 | ES 110 | M00042537A:H05 |
| ES 85 | M00057133D:F01 | ES 110 | M00042538B:E06 |
| ES 85 | M00057134A:C01 | ES 110 | M00042538D:A08 |
| ES 85 | M00057134C:A01 | ES 110 | M00042539C:E05 |
| ES 85 | M00057134D:G10 | ES 110 | M00042540A:H06 |
| ES 85 | M00057135D:H04 | ES 110 | M00042540D:F03 |
| ES 85 | M00057136A:F01 | ES 110 | M00042540D:H05 |
| ES 85 | M00057141B:B02 | ES 110 | M00042543C:H02 |
| ES 85 | M00057141D:D02 | ES 110 | M00042544B:D02 |
| ES 85 | M00057142A:A07 | ES 110 | M00042544C:F10 |
| ES 85 | M00057143C:E05 | ES 110 | M00042547A:A02 |
| ES 85 | M00057145A:D05 | ES 110 | M00042547B:D11 |
| ES 85 | M00057146D:C09 | ES 110 | M00042547C:F02 |
| ES 85 | M00057147A:A01 | ES 110 | M00042551A:D09 |
| ES 85 | M00057150A:C10 | ES 110 | M00042556A:D04 |
| ES 85 | M00057151A:B04 | ES 110 | M00042563C:E02 |
| ES 86 | M00057154A:D06 | ES 110 | M00042563C:E02 |
| ES 86 | M00057154C:B04 | ES 110 | M00042563D:G09 |
| ES 86 | M00057161B:E09 | ES 110 | M00042564B:H11 |
| ES 86 | M00057162A:C07 | ES 110 | M00042565A:H03 |
| ES 86 | M00057162B:H02 | ES 110 | M00042565C:A08 |
| ES 86 | M00057162D:D10 | ES 110 | M00042567D:C01 |
| ES 86 | M00057163D:B01 | ES 110 | M00042570D:H02 |
| ES 86 | M00057165D:E12 | ES 110 | M00042573C:A07 |
| ES 86 | M00057167B:E12 | ES 110 | M00042574B:H08 |
| ES 86 | M00057167B:G12 | ES 110 | M00042575C:D01 |
| ES 86 | M00057167D:B07 | ES 110 | M00042693D:E04 |
| ES 86 | M00057170C:H03 | ES 110 | M00042694C:E02 |
| ES 86 | M00057174B:C06 | ES 110 | M00042695B:H05 |
| ES 86 | M00057174B:G12 | ES 110 | M00042700B:A01 |
| ES 86 | M00057174C:H12 | ES 110 | M00042700B:D03 |
| ES 86 | M00057180A:H11 | ES 110 | M00042700B:D03 |
| ES 86 | M00057181C:D06 | ES 110 | M00042700D:H05 |
| ES 86 | M00057182D:B11 | ES 110 | M00042704A:F04 |
| ES 86 | M00057189B:G05 | ES 110 | M00042704A:F09 |
| ES 86 | M00057191A:A03 | ES 110 | M00042704D:E02 |
| ES 86 | M00057192B:E02 | ES 110 | M00042705A:D02 |
| ES 86 | M00057192D:G02 | ES 110 | M00042706C:A04 |
| ES 86 | M00057196A:E03 | ES 110 | M00054596B:G11 |
| ES 86 | M00057196C:F04 | ES 110 | M00004101C:H01 |
| ES 86 | M00057203C:E06 | ES 111 | M00042711C:G11 |
| ES 86 | M00057208A:A02 | ES 111 | M00042711D:C04 |
| ES 86 | M00057208C:C06 | ES 111 | M00042712B:B10 |
| ES 86 | M00057208C:D08 | ES 111 | M00042717D:D04 |
| ES 86 | M00057211B:F07 | ES 111 | M00042718B:C03 |
| ES 86 | M00057211D:A06 | ES 111 | M00042720C:D06 |
| ES 86 | M00057215B:B02 | ES 111 | M00042720D:G10 |
| ES 86 | M00057217B:B07 | ES 111 | M00042721A:G07 |
| ES 86 | M00057218D:C01 | ES 111 | M00042727C:H12 |
| ES 86 | M00057223C:C06 | ES 111 | M00042728D:E07 |
| ES 86 | M00057224B:C10 | ES 111 | M00042732A:G09 |
| ES 86 | M00057226D:C05 | ES 111 | M00042735C:G02 |
| ES 86 | M00057229D:F06 | ES 111 | M00042735D:A07 |
| ES 86 | M00057230C:D12 | ES 111 | M00042738B:D10 |
| ES 86 | M00057231C:G09 | ES 111 | M00042739D:D01 |
| ES 86 | M00057231D:A09 | ES 111 | M00042741D:D10 |
| ES 86 | M00057232B:D06 | ES 111 | M00042742B:H03 |
| ES 86 | M00057233A:F07 | ES 111 | M00042742C:A06 |
| ES 86 | M00057233B:E04 | ES 111 | M00042742D:D05 |
| ES 86 | M00057236B:H06 | ES 111 | M00042746B:F02 |
| ES 86 | M00057237A:B11 | ES 111 | M00042746D:B09 |
| ES 86 | M00057239A:G08 | ES 111 | M00042750D:B09 |
| ES 86 | M00057241B:B04 | ES 111 | M00042881D:C08 |
| ES 86 | M00057242B:F07 | ES 111 | M00042883A:F12 |
| ES 87 | M00057242D:B09 | ES 111 | M00042886C:C03 |
| ES 87 | M00057242D:H05 | ES 111 | M00042886C:F01 |
| ES 87 | M00057249A:C06 | ES 111 | M00042887C:D07 |
| ES 87 | M00057259A:H10 | ES 111 | M00042889B:A09 |
| ES 87 | M00057259B:B08 | ES 111 | M00042890D:C08 |
| ES 87 | M00057266C:D04 | ES 111 | M00042891B:C04 |
| ES 87 | M00057266C:G12 | ES 111 | M00042893B:C08 |
| ES 87 | M00057268C:E10 | ES 111 | M00042900C:C07 |
| ES 87 | M00057270B:H09 | ES 111 | M00042901B:A03 |
| ES 87 | M00057270C:E04 | ES 111 | M00042902A:C04 |
| ES 87 | M00057271C:E01 | ES 111 | M00042905A:F11 |
| ES 87 | M00057272A:B03 | ES 111 | M00042905C:C10 |
| ES 87 | M00057272C:H04 | ES 111 | M00042908D:G01 |
| ES 87 | M00057272D:A01 | ES 111 | M00042909B:G04 |
| ES 87 | M00057275B:A12 | ES 111 | M00042911A:H03 |
| ES 87 | M00057277B:C09 | ES 111 | M00042914D:B10 |
| ES 87 | M00057277B:E10 | ES 111 | M00054792D:E09 |
| ES 87 | M00057279A:G02 | ES 111 | M00054793D:B07 |
| ES 87 | M00057280C:A06 | ES 111 | M00054798D:F01 |
| ES 87 | M00057283A:E06 | ES 111 | M00054913C:G03 |
| ES 87 | M00057288D:E08 | ES 111 | M00054915D:E07 |
| ES 87 | M00057291C:B06 | ES 111 | M00054917B:F09 |
| ES 87 | M00057297A:F03 | ES 111 | M00054917D:D12 |
| ES 87 | M00057300B:F02 | ES 111 | M00054918C:D03 |
| ES 87 | M00057301B:H12 | ES 112 | M00054918D:C11 |
| ES 87 | M00057304A:E01 | ES 112 | M00055426B:B02 |
| ES 87 | M00057306B:H07 | ES 112 | M00055426C:H06 |
| ES 87 | M00057312B:E11 | ES 112 | M00055427A:F01 |
| ES 87 | M00057318B:B09 | ES 112 | M00055428C:A02 |
| ES 87 | M00057318C:A03 | ES 112 | M00055429A:H05 |
| ES 87 | M00057324A:D12 | ES 112 | M00055430B:H02 |
| ES 87 | M00057325C:C10 | ES 112 | M00055431C:E09 |
| ES 87 | M00057333A:F09 | ES 112 | M00055438C:C06 |
| ES 87 | M00057334B:F01 | ES 112 | M00055438C:H10 |
| ES 87 | M00057337B:G02 | ES 112 | M00055441B:D02 |
| ES 87 | M00057340B:C12 | ES 112 | M00055445D:G06 |
| ES 87 | M00042355A:G02 | ES 112 | M00055446C:B06 |
| ES 87 | M00042355D:C01 | ES 112 | M00055447D:H04 |
| ES 87 | M00042442D:A02 | ES 112 | M00055447D:H04 |
| ES 87 | M00042444D:G05 | ES 112 | M00055448A:D08 |
| ES 87 | M00042444D:H08 | ES 112 | M00055448C:E07 |
| ES 87 | M00042450D:H10 | ES 112 | M00055450A:G09 |
| ES 87 | M00042453C:E01 | ES 112 | M00055450D:B08 |
| ES 87 | M00042460D:A07 | ES 112 | M00055451A:F07 |
| ES 87 | M00042517C:F07 | ES 112 | M00055451A:F11 |
| ES 87 | M00042518D:A06 | ES 112 | M00055451C:G11 |
| ES 87 | M00042520A:F04 | ES 112 | M00055453C:E01 |
| ES 88 | M00042520A:F09 | ES 112 | M00055453C:E01 |
| ES 88 | M00042520A:F09 | ES 112 | M00055454A:A07 |
| ES 88 | M00043296C:B10 | ES 112 | M00055454A:H11 |
| ES 88 | M00043300A:H11 | ES 112 | M00055454C:G05 |
| ES 88 | M00043301A:F06 | ES 112 | M00055456D:F12 |
| ES 88 | M00043301D:H09 | ES 112 | M00055463D:H10 |
| ES 88 | M00043304A:D01 | ES 112 | M00055464A:F05 |
| ES 88 | M00043304B:C05 | ES 112 | M00055466D:B08 |
| ES 88 | M00043304B:C05 | ES 112 | M00055470B:G01 |
| ES 88 | M00043306D:B07 | ES 112 | M00055491A:G08 |
| ES 88 | M00043309B:H07 | ES 112 | M00055494D:C09 |
| ES 88 | M00043310C:B03 | ES 112 | M00055495A:G02 |
| ES 88 | M00043313A:A03 | ES 112 | M00055495C:D05 |
| ES 88 | M00043313A:G07 | ES 112 | M00055495C:F03 |
| ES 88 | M00043313D:C06 | ES 112 | M00055495D:E02 |
| ES 88 | M00043314C:H04 | ES 112 | M00055496A:F09 |
| ES 88 | M00043317A:H01 | ES 112 | M00055496B:E07 |
| ES 88 | M00043317C:F04 | ES 112 | M00055496C:C09 |
| ES 88 | M00043323C:D04 | ES 112 | M00055498A:H09 |
| ES 88 | M00043324D:D04 | ES 112 | M00055500D:B05 |
| ES 88 | M00043327D:H02 | ES 112 | M00055504C:D08 |
| ES 88 | M00043327D:H02 | ES 112 | M00055505D:A10 |
| ES 88 | M00043336B:E08 | ES 112 | M00055508D:E03 |
| ES 88 | M00043338A:B03 | ES 112 | M00055509C:H09 |
| ES 88 | M00043338B:A03 | ES 112 | M00055510B:B07 |
| ES 88 | M00043345B:C03 | ES 113 | M00055511D:E09 |
| ES 88 | M00043347B:G12 | ES 113 | M00055512C:G06 |
| ES 88 | M00043349A:C08 | ES 113 | M00055512D:D07 |
| ES 88 | M00043350B:H06 | ES 113 | M00055512D:F08 |
| ES 88 | M00043350C:H09 | ES 113 | M00055513C:D06 |
| ES 88 | M00043352A:E09 | ES 113 | M00055514D:H05 |
| ES 88 | M00043352D:B05 | ES 113 | M00055516B:E08 |
| ES 88 | M00043354D:C01 | ES 113 | M00055517B:D03 |
| ES 88 | M00043355D:H11 | ES 113 | M00055519B:C06 |
| ES 88 | M00043361D:D05 | ES 113 | M00055519C:H07 |
| ES 88 | M00043365A:C06 | ES 113 | M00055520C:A06 |
| ES 88 | M00043374A:B02 | ES 113 | M00055522A:E07 |
| ES 88 | M00043374B:B06 | ES 113 | M00055522D:C02 |
| ES 88 | M00043377A:C03 | ES 113 | M00055522D:C02 |
| ES 88 | M00043379D:C07 | ES 113 | M00055523D:C03 |
| ES 88 | M00043381B:E10 | ES 113 | M00055525C:B07 |
| ES 88 | M00043386D:A06 | ES 113 | M00055526D:F09 |
| ES 88 | M00043388D:C09 | ES 113 | M00055527C:E02 |
| ES 88 | M00043394D:B06 | ES 113 | M00055527C:E04 |
| ES 88 | M00043397B:B02 | ES 113 | M00055527D:G11 |
| ES 88 | M00043397C:B09 | ES 113 | M00055528C:F06 |
| ES 88 | M00043503C:C08 | ES 113 | M00055529D:B02 |
| ES 88 | M00043503C:E05 | ES 113 | M00055530D:B07 |
| ES 89 | M00043504C:G06 | ES 113 | M00055532C:G08 |
| ES 89 | M00043504D:G08 | ES 113 | M00055534C:H01 |
| ES 89 | M00043506A:H09 | ES 113 | M00055536C:E06 |
| ES 89 | M00043507A:D05 | ES 113 | M00055536C:F03 |
| ES 89 | M00043508A:A08 | ES 113 | M00055538B:H11 |
| ES 89 | M00043508D:C01 | ES 113 | M00055542C:C01 |
| ES 89 | M00054486A:B11 | ES 113 | M00055542C:F06 |
| ES 89 | M00054493A:A10 | ES 113 | M00055542D:A09 |
| ES 89 | M00054494A:E01 | ES 113 | M00055543A:C05 |
| ES 89 | M00054496A:B09 | ES 113 | M00055543A:C05 |
| ES 89 | M00054499B:E11 | ES 113 | M00055543C:G08 |
| ES 89 | M00054499B:E11 | ES 113 | M00055544A:E04 |
| ES 89 | M00054502A:D01 | ES 113 | M00055544B:B02 |
| ES 89 | M00054502C:E02 | ES 113 | M00055545C:H12 |
| ES 89 | M00054507A:C11 | ES 113 | M00055547D:D10 |
| ES 89 | M00054510D:H09 | ES 113 | M00055547D:E05 |
| ES 89 | M00054513A:A12 | ES 113 | M00055548A:F04 |
| ES 89 | M00054518D:D03 | ES 113 | M00055548C:E12 |
| ES 89 | M00054520C:B05 | ES 113 | M00055548C:E12 |
| ES 89 | M00054521D:F04 | ES 113 | M00055552A:C09 |
| ES 89 | M00054522B:H11 | ES 113 | M00055553B:H04 |
| ES 89 | M00054523D:A10 | ES 113 | M00055553D:C07 |
| ES 89 | M00054524D:B02 | ES 113 | M00055553D:H02 |
| ES 89 | M00054534D:D02 | ES 113 | M00055556C:H09 |
| ES 89 | M00054535C:H09 | ES 113 | M00055560B:B12 |
| ES 89 | M00054542C:A08 | ES 114 | M00055560B:F02 |
| ES 89 | M00054551C:G03 | ES 114 | M00055560C:F06 |
| ES 89 | M00054555C:G12 | ES 114 | M00055563A:A02 |
| ES 89 | M00054561D:E06 | ES 114 | M00055572A:B12 |
| ES 89 | M00054563B:C09 | ES 114 | M00055572C:F03 |
| ES 89 | M00054568A:G11 | ES 114 | M00055575A:D08 |
| ES 89 | M00054569A:H07 | ES 114 | M00055578A:H09 |
| ES 89 | M00054571C:C01 | ES 114 | M00055581D:B01 |
| ES 89 | M00054572B:C01 | ES 114 | M00055582B:E04 |
| ES 89 | M00054575C:C01 | ES 114 | M00055583B:B04 |
| ES 89 | M00054580C:D11 | ES 114 | M00055583B:H05 |
| ES 89 | M00054583A:F05 | ES 114 | M00055584A:C11 |
| ES 89 | M00054587A:F09 | ES 114 | M00055584D:G06 |
| ES 89 | M00054590C:G02 | ES 114 | M00055586C:F05 |
| ES 89 | M00054591C:H07 | ES 114 | M00055586D:F02 |
| ES 89 | M00054595A:B02 | ES 114 | M00055591C:H01 |
| ES 89 | M00054595B:H09 | ES 114 | M00055592D:A05 |
| ES 89 | M00054596B:B07 | ES 114 | M00055594B:A01 |
| ES 89 | M00054600D:G07 | ES 114 | M00055597C:E08 |
| ES 89 | M00054601A:H10 | ES 114 | M00055601C:D09 |
| ES 89 | M00054601D:E08 | ES 114 | M00055602B:G10 |
| ES 89 | M00054602A:C04 | ES 114 | M00055602C:E07 |
| ES 90 | M00054602B:D02 | ES 114 | M00055609A:G03 |
| ES 90 | M00054604A:D09 | ES 114 | M00055609D:F12 |
| ES 90 | M00054604A:D09 | ES 114 | M00055613A:D10 |
| ES 90 | M00054605C:D01 | ES 114 | M00055613A:E02 |
| ES 90 | M00054609A:F01 | ES 114 | M00055618C:A06 |
| ES 90 | M00054609D:H06 | ES 114 | M00055628A:A08 |
| ES 90 | M00054611C:F02 | ES 114 | M00055630B:E09 |
| ES 90 | M00054613A:D09 | ES 114 | M00055633D:A02 |
| ES 90 | M00054613A:D09 | ES 114 | M00055633D:G11 |
| ES 90 | M00054617B:A09 | ES 114 | M00055635A:H10 |
| ES 90 | M00054621B:C06 | ES 114 | M00055635B:E10 |
| ES 90 | M00054621D:D11 | ES 114 | M00055635C:G04 |
| ES 90 | M00054629C:E09 | ES 114 | M00055636A:F10 |
| ES 90 | M00054636B:B03 | ES 114 | M00055647C:B04 |
| ES 90 | M00054636C:A02 | ES 114 | M00055653A:H04 |
| ES 90 | M00054636C:F02 | ES 114 | M00055656A:E09 |
| ES 90 | M00054638A:D09 | ES 114 | M00055662C:A04 |
| ES 90 | M00054638B:C08 | ES 114 | M00055664C:A08 |
| ES 90 | M00054646C:B01 | ES 114 | M00055668B:B07 |
| ES 90 | M00054647D:H02 | ES 114 | M00055679A:A07 |
| ES 90 | M00054648C:H10 | ES 114 | M00055681B:G02 |
| ES 90 | M00054660D:F05 | ES 114 | M00055685D:E01 |
| ES 90 | M00054665B:H08 | ES 114 | M00055686D:E11 |
| ES 90 | M00054665D:E11 | ES 114 | M00055687C:F01 |
| ES 90 | M00054677C:D02 | ES 114 | M00055688C:B04 |
| ES 90 | M00054678A:E07 | ES 114 | M00055689A:G12 |
| ES 90 | M00054679B:D12 | ES 115 | M00055689D:F02 |
| ES 90 | M00054680B:E06 | ES 115 | M00055689D:F07 |
| ES 90 | M00054680D:B11 | ES 115 | M00055691B:E07 |
| ES 90 | M00054681C:B02 | ES 115 | M00055692D:E07 |
| ES 90 | M00054684C:H12 | ES 115 | M00055701C:D10 |
| ES 90 | M00054689D:E12 | ES 115 | M00055703A:B08 |
| ES 90 | M00054691A:E05 | ES 115 | M00055703B:B06 |
| ES 90 | M00054692B:D01 | ES 115 | M00055703B:C05 |
| ES 90 | M00054694D:G04 | ES 115 | M00055703C:G09 |
| ES 90 | M00054706B:C09 | ES 115 | M00055704C:D07 |
| ES 90 | M00054707B:B08 | ES 115 | M00055705C:G07 |
| ES 90 | M00054707B:E05 | ES 115 | M00055706A:A01 |
| ES 90 | M00054713A:D12 | ES 115 | M00055706B:G01 |
| ES 90 | M00054720D:D12 | ES 115 | M00055707D:C08 |
| ES 90 | M00054720D:F11 | ES 115 | M00055709B:G09 |
| ES 90 | M00054721C:F11 | ES 115 | M00055716C:B04 |
| ES 90 | M00054722C:D01 | ES 115 | M00055717B:F04 |
| ES 90 | M00054722D:C08 | ES 115 | M00055718A:F05 |
| ES 90 | M00054726A:F08 | ES 115 | M00055720B:G09 |
| ES 90 | M00054727D:E10 | ES 115 | M00055720C:A06 |
| ES 90 | M00054727D:H06 | ES 115 | M00055720D:A01 |
| ES 90 | M00054728B:E08 | ES 115 | M00055721B:F06 |
| ES 91 | M00054728D:B10 | ES 115 | M00055721B:F06 |
| ES 91 | M00054729A:E01 | ES 115 | M00055721C:E05 |
| ES 91 | M00054731C:C12 | ES 115 | M00055723A:B08 |
| ES 91 | M00054732D:E03 | ES 115 | M00055723D:E05 |
| ES 91 | M00054734D:H10 | ES 115 | M00055724D:D09 |
| ES 91 | M00054739A:G03 | ES 115 | M00055726B:B08 |
| ES 91 | M00054739C:D03 | ES 115 | M00055726C:D12 |
| ES 91 | M00054739C:E06 | ES 115 | M00055726C:G10 |
| ES 91 | M00054740A:H08 | ES 115 | M00055729D:A06 |
| ES 91 | M00054741A:C10 | ES 115 | M00055731A:H12 |
| ES 91 | M00054741A:E10 | ES 115 | M00055733A:G11 |
| ES 91 | M00054741D:G10 | ES 115 | M00055734C:H05 |
| ES 91 | M00054743C:E02 | ES 115 | M00055735C:C07 |
| ES 91 | M00054745D:A03 | ES 115 | M00055735C:G05 |
| ES 91 | M00054747A:F01 | ES 115 | M00055736A:D06 |
| ES 91 | M00054747D:C06 | ES 115 | M00055736B:G03 |
| ES 91 | M00054750C:D12 | ES 115 | M00055736C:G07 |
| ES 91 | M00054752B:A07 | ES 115 | M00055740B:B12 |
| ES 91 | M00054755B:H06 | ES 115 | M00055740B:F09 |
| ES 91 | M00054759A:B08 | ES 115 | M00055743B:C12 |
| ES 91 | M00054760A:A12 | ES 115 | M00055744B:C08 |
| ES 91 | M00054762B:F07 | ES 115 | M00055744C:F08 |
| ES 91 | M00054765B:C05 | ES 115 | M00055744C:F09 |
| ES 91 | M00054766C:B04 | ES 115 | M00055744D:G08 |
| ES 91 | M00054769A:F07 | ES 115 | M00055747C:D09 |
| ES 91 | M00054772C:C06 | ES 115 | M00055749D:H11 |
| ES 91 | M00054773A:A12 | ES 116 | M00055751C:D01 |
| ES 91 | M00054776B:F01 | ES 116 | M00055755C:D02 |
| ES 91 | M00054779A:F07 | ES 116 | M00055755D:H03 |
| ES 91 | M00054780C:G08 | ES 116 | M00055761D:C03 |
| ES 91 | M00054781B:B04 | ES 116 | M00055763B:E06 |
| ES 91 | M00054802A:G02 | ES 116 | M00055765A:B05 |
| ES 91 | M00054804D:H12 | ES 116 | M00055766A:H03 |
| ES 91 | M00054808A:D07 | ES 116 | M00055768A:B05 |
| ES 91 | M00054808B:F08 | ES 116 | M00055770C:G01 |
| ES 91 | M00054810B:H02 | ES 116 | M00055771A:A11 |
| ES 91 | M00054812B:A05 | ES 116 | M00055771A:D01 |
| ES 91 | M00054812D:C07 | ES 116 | M00055771C:A11 |
| ES 91 | M00054812D:C07 | ES 116 | M00055771C:F05 |
| ES 91 | M00054815C:E01 | ES 116 | M00055771D:D04 |
| ES 91 | M00054816C:D11 | ES 116 | M00055771D:F07 |
| ES 91 | M00054821A:C11 | ES 116 | M00055774C:E01 |
| ES 91 | M00054823D:H07 | ES 116 | M00055774D:G03 |
| ES 91 | M00054826B:C10 | ES 116 | M00055775C:B10 |
| ES 91 | M00054826B:E05 | ES 116 | M00055775D:C06 |
| ES 91 | M00054826D:C10 | ES 116 | M00055778A:F09 |
| ES 91 | M00054827B:H01 | ES 116 | M00055779B:A02 |
| ES 92 | M00054832D:E09 | ES 116 | M00055780B:B07 |
| ES 92 | M00054836A:B05 | ES 116 | M00055780D:G08 |
| ES 92 | M00054839B:B02 | ES 116 | M00055781C:C05 |
| ES 92 | M00054839C:F06 | ES 116 | M00055782A:F02 |
| ES 92 | M00054841D:B07 | ES 116 | M00055783A:C11 |
| ES 92 | M00054841D:B07 | ES 116 | M00055785B:F03 |
| ES 92 | M00054842D:C11 | ES 116 | M00055785C:E08 |
| ES 92 | M00054844D:F06 | ES 116 | M00055786A:D05 |
| ES 92 | M00054849D:H11 | ES 116 | M00055788D:A03 |
| ES 92 | M00054851B:E03 | ES 116 | M00055790C:H02 |
| ES 92 | M00054854D:E08 | ES 116 | M00055791A:D05 |
| ES 92 | M00054856D:A02 | ES 116 | M00055791D:F03 |
| ES 92 | M00054857D:E12 | ES 116 | M00055792B:G09 |
| ES 92 | M00054862B:B07 | ES 116 | M00055792D:E07 |
| ES 92 | M00054863B:G03 | ES 116 | M00055794A:G11 |
| ES 92 | M00054865B:H04 | ES 116 | M00055794C:B06 |
| ES 92 | M00054866C:G07 | ES 116 | M00055796D:E10 |
| ES 92 | M00054867A:C07 | ES 116 | M00055797A:D08 |
| ES 92 | M00054867B:B02 | ES 116 | M00055797B:E07 |
| ES 92 | M00054867C:B07 | ES 116 | M00055798B:C06 |
| ES 92 | M00054869C:D01 | ES 116 | M00055800A:D08 |
| ES 92 | M00054870B:D09 | ES 116 | M00055800B:C08 |
| ES 92 | M00054875B:C04 | ES 116 | M00055802A:D08 |
| ES 92 | M00054876B:G03 | ES 116 | M00055802A:G02 |
| ES 92 | M00054877A:H12 | ES 116 | M00055802B:H04 |
| ES 92 | M00054895B:D09 | ES 116 | M00055802C:F12 |
| ES 92 | M00054899D:F07 | ES 116 | M00055803B:A11 |
| ES 92 | M00054899D:G01 | ES 117 | M00055803C:D08 |
| ES 92 | M00054903D:C12 | ES 117 | M00055804A:F03 |
| ES 92 | M00054908B:F07 | ES 117 | M00055804B:F01 |
| ES 92 | M00054910D:G06 | ES 117 | M00055805B:C07 |
| ES 92 | M00054926D:F01 | ES 117 | M00055805C:D10 |
| ES 92 | M00054927B:E08 | ES 117 | M00055806A:H12 |
| ES 92 | M00054931C:A09 | ES 117 | M00055806B:F07 |
| ES 92 | M00054933A:D07 | ES 117 | M00055806C:E09 |
| ES 92 | M00054934C:D03 | ES 117 | M00055807B:G10 |
| ES 92 | M00054935A:E01 | ES 117 | M00055807B:G10 |
| ES 92 | M00054935A:G04 | ES 117 | M00055807D:C04 |
| ES 92 | M00054937A:B03 | ES 117 | M00055808C:G11 |
| ES 92 | M00054937B:A12 | ES 117 | M00055811A:A08 |
| ES 92 | M00054937B:F03 | ES 117 | M00055811D:C12 |
| ES 92 | M00054937C:B10 | ES 117 | M00055812A:E01 |
| ES 92 | M00054941C:G04 | ES 117 | M00055814C:D11 |
| ES 92 | M00054943C:C04 | ES 117 | M00055816B:F01 |
| ES 92 | M00054943D:C03 | ES 117 | M00055817B:F01 |
| ES 92 | M00054945C:G07 | ES 117 | M00055817C:C08 |
| ES 92 | M00054947B:G12 | ES 117 | M00055817C:D08 |
| ES 92 | M00054949A:E03 | ES 117 | M00055818A:F12 |
| ES 93 | M00054949C:A07 | ES 117 | M00055818D:E10 |
| ES 93 | M00054950D:G06 | ES 117 | M00055820A:E08 |
| ES 93 | M00054952A:F01 | ES 117 | M00055820B:E05 |
| ES 93 | M00054952C:H06 | ES 117 | M00055820C:C08 |
| ES 93 | M00054953D:G10 | ES 117 | M00055820D:G10 |
| ES 93 | M00054954B:C03 | ES 117 | M00055821A:A06 |
| ES 93 | M00054954D:F01 | ES 117 | M00055821A:G12 |
| ES 93 | M00054957A:B02 | ES 117 | M00055822B:H04 |
| ES 93 | M00054959C:C11 | ES 117 | M00055823B:D03 |
| ES 93 | M00054963C:H11 | ES 117 | M00055823C:D11 |
| ES 93 | M00054963D:H04 | ES 117 | M00055825B:E03 |
| ES 93 | M00054964A:H11 | ES 117 | M00055826A:F04 |
| ES 93 | M00054965B:H02 | ES 117 | M00055827B:D02 |
| ES 93 | M00054970D:G03 | ES 117 | M00055827D:A02 |
| ES 93 | M00054973B:A10 | ES 117 | M00055827D:C06 |
| ES 93 | M00054975C:C04 | ES 117 | M00055827D:E08 |
| ES 93 | M00054980D:C02 | ES 117 | M00055829C:G09 |
| ES 93 | M00054981C:E11 | ES 117 | M00055830A:G10 |
| ES 93 | M00054981D:C06 | ES 117 | M00055832D:E12 |
| ES 93 | M00054984D:B12 | ES 117 | M00055833D:A11 |
| ES 93 | M00054984D:C07 | ES 117 | M00055838B:D12 |
| ES 93 | M00054985C:F07 | ES 117 | M00055838B:G12 |
| ES 93 | M00054987D:C02 | ES 117 | M00055839A:F09 |
| ES 93 | M00054988C:G02 | ES 117 | M00055839B:A10 |
| ES 93 | M00054995A:C10 | ES 117 | M00055840C:D06 |
| ES 93 | M00054996C:B11 | ES 117 | M00055841A:B09 |
| ES 93 | M00054996C:C09 | ES 117 | M00055841C:D05 |
| ES 93 | M00054997C:B12 | ES 118 | M00055848C:H06 |
| ES 93 | M00054997C:H03 | ES 118 | M00055849C:D08 |
| ES 93 | M00055000C:F04 | ES 118 | M00055850B:F03 |
| ES 93 | M00055002D:E04 | ES 118 | M00055851A:G11 |
| ES 93 | M00055005B:H11 | ES 118 | M00055851C:F12 |
| ES 93 | M00055005D:B08 | ES 118 | M00055851C:F12 |
| ES 93 | M00055008A:B08 | ES 118 | M00055852A:A07 |
| ES 93 | M00055008D:B09 | ES 118 | M00055854A:E04 |
| ES 93 | M00055011C:E04 | ES 118 | M00055856A:F04 |
| ES 93 | M00055017A:A11 | ES 118 | M00055856C:F07 |
| ES 93 | M00055021D:D11 | ES 118 | M00055860D:A08 |
| ES 93 | M00055022A:H04 | ES 118 | M00055861A:D03 |
| ES 93 | M00055027B:D07 | ES 118 | M00055864A:E11 |
| ES 93 | M00055027D:F08 | ES 118 | M00055864A:H02 |
| ES 93 | M00055032D:A06 | ES 118 | M00055866D:A02 |
| ES 93 | M00055034C:G01 | ES 118 | M00055868A:F06 |
| ES 93 | M00055034D:H01 | ES 118 | M00055868D:D03 |
| ES 93 | M00055037A:E10 | ES 118 | M00055868D:F09 |
| ES 93 | M00055039A:G01 | ES 118 | M00055869B:A06 |
| ES 93 | M00055039C:E02 | ES 118 | M00055871A:F05 |
| ES 93 | M00055041A:E02 | ES 118 | M00055871D:G06 |
| ES 94 | M00055042A:B01 | ES 118 | M00055872D:D12 |
| ES 94 | M00055046B:C07 | ES 118 | M00055873A:B11 |
| ES 94 | M00055046C:E11 | ES 118 | M00055873B:E03 |
| ES 94 | M00055050C:G04 | ES 118 | M00055874B:B06 |
| ES 94 | M00055053C:B03 | ES 118 | M00055874D:D03 |
| ES 94 | M00055054A:C02 | ES 118 | M00055879B:E11 |
| ES 94 | M00055056D:B06 | ES 118 | M00055879C:D04 |
| ES 94 | M00055057A:F03 | ES 118 | M00055880D:F12 |
| ES 94 | M00055063D:G01 | ES 118 | M00055882C:A06 |
| ES 94 | M00055064A:E12 | ES 118 | M00055882C:A09 |
| ES 94 | M00055071B:A02 | ES 118 | M00055884A:E10 |
| ES 94 | M00055073C:H12 | ES 118 | M00055884C:B01 |
| ES 94 | M00055075B:H05 | ES 118 | M00055884D:A05 |
| ES 94 | M00055077C:F11 | ES 118 | M00055886D:G09 |
| ES 94 | M00055085A:A10 | ES 118 | M00055887A:C06 |
| ES 94 | M00055087A:A10 | ES 118 | M00055887A:F07 |
| ES 94 | M00055088A:A12 | ES 118 | M00055887B:E04 |
| ES 94 | M00055088C:E09 | ES 118 | M00055888B:B05 |
| ES 94 | M00055093B:H05 | ES 118 | M00055889C:H12 |
| ES 94 | M00055094B:H09 | ES 118 | M00055891B:A04 |
| ES 94 | M00055097A:G06 | ES 118 | M00055893B:C05 |
| ES 94 | M00055100B:D08 | ES 118 | M00055896C:F11 |
| ES 94 | M00055104C:B12 | ES 118 | M00055900B:B05 |
| ES 94 | M00055106A:D07 | ES 118 | M00055906B:D10 |
| ES 94 | M00055111B:D03 | ES 118 | M00055906C:F12 |
| ES 94 | M00055112A:C03 | ES 118 | M00055908B:H11 |
| ES 94 | M00055113B:A11 | ES 118 | M00055908C:E12 |
| ES 94 | M00055114D:A11 | ES 122 | M00056209B:F12 |
| ES 94 | M00055115A:E05 | ES 122 | M00056210B:E03 |
| ES 94 | M00055116B:B02 | ES 122 | M00056212C:G01 |
| ES 94 | M00055117C:C03 | ES 122 | M00056213A:A04 |
| ES 94 | M00055121D:C07 | ES 122 | M00056215A:E03 |
| ES 94 | M00055125B:E06 | ES 122 | M00056218C:G03 |
| ES 94 | M00055125B:F01 | ES 122 | M00056220D:D02 |
| ES 94 | M00055128D:B10 | ES 122 | M00056220D:D09 |
| ES 94 | M00055130D:G01 | ES 122 | M00056221D:E05 |
| ES 94 | M00055131C:B10 | ES 122 | M00056222C:F02 |
| ES 94 | M00055134B:E03 | ES 122 | M00056223B:G03 |
| ES 94 | M00055134B:H02 | ES 122 | M00056224C:B10 |
| ES 94 | M00055134D:B03 | ES 122 | M00056224D:E08 |
| ES 94 | M00055137C:C04 | ES 122 | M00056225D:G09 |
| ES 94 | M00055145A:F07 | ES 122 | M00056226C:F12 |
| ES 94 | M00055148D:D11 | ES 122 | M00056228B:A07 |
| ES 94 | M00055154C:F04 | ES 122 | M00056231B:G09 |
| ES 94 | M00055157A:C11 | ES 122 | M00056231B:G09 |
| ES 94 | M00055161D:A11 | ES 122 | M00056232C:E06 |
| ES 94 | M00055162A:F06 | ES 122 | M00056232D:G12 |
| ES 94 | M00055163A:C02 | ES 122 | M00056233D:F03 |
| ES 95 | M00055170A:F01 | ES 122 | M00056236D:G01 |
| ES 95 | M00055170D:E02 | ES 122 | M00056238B:E07 |
| ES 95 | M00055172D:D04 | ES 122 | M00056243C:G10 |
| ES 95 | M00055179C:D02 | ES 122 | M00056244A:B08 |
| ES 95 | M00055181A:E01 | ES 122 | M00056244B:C07 |
| ES 95 | M00055182B:C07 | ES 122 | M00056246A:B03 |
| ES 95 | M00055185C:B01 | ES 122 | M00056246C:G07 |
| ES 95 | M00055194D:C05 | ES 122 | M00056247A:G06 |
| ES 95 | M00055196B:A09 | ES 122 | M00056248A:A09 |
| ES 95 | M00055198D:F07 | ES 122 | M00056250B:F01 |
| ES 95 | M00055198D:G07 | ES 122 | M00056251C:A06 |
| ES 95 | M00055201D:A03 | ES 122 | M00056252B:H08 |
| ES 95 | M00055201D:B07 | ES 122 | M00056252D:E11 |
| ES 95 | M00055203B:H02 | ES 122 | M00056253A:F12 |
| ES 95 | M00055206A:H04 | ES 122 | M00056253D:H06 |
| ES 95 | M00055207D:A04 | ES 122 | M00056254A:H02 |
| ES 95 | M00055209D:A08 | ES 122 | M00056256C:H11 |
| ES 95 | M00055209D:D10 | ES 122 | M00056262B:B08 |
| ES 95 | M00055216A:A03 | ES 122 | M00056263D:C01 |
| ES 95 | M00055216A:A03 | ES 122 | M00056267A:E02 |
| ES 95 | M00055222D:H05 | ES 122 | M00056267C:B02 |
| ES 95 | M00055227A:H09 | ES 122 | M00056268B:B04 |
| ES 95 | M00055227D:E02 | ES 122 | M00056268C:D01 |
| ES 95 | M00055227D:E07 | ES 122 | M00056273B:A01 |
| ES 95 | M00055231A:D10 | ES 122 | M00056281D:E04 |
| ES 95 | M00055242A:E06 | ES 122 | M00056282A:F11 |
| ES 95 | M00055242B:A01 | ES 122 | M00056282B:D04 |
| ES 95 | M00055242D:D04 | ES 123 | M00056282D:C01 |
| ES 95 | M00055243A:F04 | ES 123 | M00056282D:H09 |
| ES 95 | M00055243A:G01 | ES 123 | M00056283A:E02 |
| ES 95 | M00055245B:A09 | ES 123 | M00056283A:E02 |
| ES 95 | M00055247B:A11 | ES 123 | M00056283D:C03 |
| ES 95 | M00055252A:C02 | ES 123 | M00056286A:E08 |
| ES 95 | M00055259D:F04 | ES 123 | M00056286D:A12 |
| ES 95 | M00055260B:A05 | ES 123 | M00056290B:F05 |
| ES 95 | M00055260C:F12 | ES 123 | M00056290D:E08 |
| ES 95 | M00055262C:B11 | ES 123 | M00056290D:H02 |
| ES 95 | M00055263A:G09 | ES 123 | M00056291B:G01 |
| ES 95 | M00055271D:C05 | ES 123 | M00056291D:B05 |
| ES 95 | M00055273B:C05 | ES 123 | M00056292B:E05 |
| ES 95 | M00055274C:F02 | ES 123 | M00056293B:E08 |
| ES 95 | M00055279B:G08 | ES 123 | M00056293C:F05 |
| ES 95 | M00055279C:E12 | ES 123 | M00056293C:G09 |
| ES 95 | M00055283B:F05 | ES 123 | M00056295A:F07 |
| ES 95 | M00055283C:H02 | ES 123 | M00056295C:D06 |
| ES 95 | M00055289B:D02 | ES 123 | M00056300A:A05 |
| ES 95 | M00055294B:D04 | ES 123 | M00056302B:F12 |
| ES 96 | M00055302D:F02 | ES 123 | M00056303A:C03 |
| ES 96 | M00055306A:G09 | ES 123 | M00056303C:B04 |
| ES 96 | M00055319B:A01 | ES 123 | M00056304D:G11 |
| ES 96 | M00055322B:E01 | ES 123 | M00056307A:H12 |
| ES 96 | M00055324C:H10 | ES 123 | M00056310B:G06 |
| ES 96 | M00055325C:B12 | ES 123 | M00056312B:A04 |
| ES 96 | M00055327D:H08 | ES 123 | M00056312D:C03 |
| ES 96 | M00055332C:G11 | ES 123 | M00056313C:F07 |
| ES 96 | M00055334C:H09 | ES 123 | M00056319C:G01 |
| ES 96 | M00055335A:H03 | ES 123 | M00056320C:H02 |
| ES 96 | M00055338B:H07 | ES 123 | M00056323A:H10 |
| ES 96 | M00055344C:H09 | ES 123 | M00056323A:H10 |
| ES 96 | M00055345C:H11 | ES 123 | M00056323C:C12 |
| ES 96 | M00055346B:D02 | ES 123 | M00056323D:A07 |
| ES 96 | M00055350A:F01 | ES 123 | M00056324B:D02 |
| ES 96 | M00055356C:C06 | ES 123 | M00056330C:D03 |
| ES 96 | M00055358B:C01 | ES 123 | M00056331B:D01 |
| ES 96 | M00055361D:H01 | ES 123 | M00056338B:B06 |
| ES 96 | M00055363D:G12 | ES 123 | M00056338C:B10 |
| ES 96 | M00055364D:E01 | ES 123 | M00056342A:G05 |
| ES 96 | M00055368B:C10 | ES 123 | M00056342B:G03 |
| ES 96 | M00055368C:B06 | ES 123 | M00056342C:F11 |
| ES 96 | M00055371B:F01 | ES 123 | M00056344A:G03 |
| ES 96 | M00055373D:D10 | ES 123 | M00056345D:E03 |
| ES 96 | M00055374A:A08 | ES 123 | M00056437C:H07 |
| ES 96 | M00055376B:B01 | ES 123 | M00056438C:A06 |
| ES 96 | M00055379D:C08 | ES 123 | M00056447B:A04 |
| ES 96 | M00055381B:E09 | ES 123 | M00056448B:C09 |
| ES 96 | M00055383B:B04 | ES 124 | M00056456C:A09 |
| ES 96 | M00055383B:B04 | ES 124 | M00056456C:F02 |
| ES 96 | M00055384D:A03 | ES 124 | M00056456D:F01 |
| ES 96 | M00055385C:G06 | ES 124 | M00056459A:C07 |
| ES 96 | M00055388A:G08 | ES 124 | M00056459A:C07 |
| ES 96 | M00055388A:H08 | ES 124 | M00056459A:D07 |
| ES 96 | M00055390B:D08 | ES 124 | M00056460A:G11 |
| ES 96 | M00055391A:G08 | ES 124 | M00056466A:A03 |
| ES 96 | M00055391C:G06 | ES 124 | M00056466A:E02 |
| ES 96 | M00055395A:C02 | ES 124 | M00056467C:E07 |
| ES 96 | M00055396A:G07 | ES 124 | M00056475B:C12 |
| ES 96 | M00055404D:C07 | ES 124 | M00056475C:F01 |
| ES 96 | M00055405A:D09 | ES 124 | M00056475C:F02 |
| ES 96 | M00055405B:H05 | ES 124 | M00042432C:H10 |
| ES 96 | M00055405D:G07 | ES 124 | M00042440A:E05 |
| ES 96 | M00055406B:D05 | ES 124 | M00042461A:A10 |
| ES 96 | M00055408B:E09 | ES 124 | M00042463A:F09 |
| ES 96 | M00055408D:F03 | ES 124 | M00042466D:H06 |
| ES 96 | M00055413A:B07 | ES 124 | M00042469D:H04 |
| ES 96 | M00055414C:A11 | ES 124 | M00042511A:A04 |
| ES 97 | M00055415B:H11 | ES 124 | M00042513D:A12 |
| ES 97 | M00055417A:G08 | ES 124 | M00042515C:A10 |
| ES 97 | M00055419D:G01 | ES 124 | M00042756B:B01 |
| ES 97 | M00055420A:E06 | ES 124 | M00042758D:H12 |
| ES 97 | M00055420B:F10 | ES 124 | M00042760B:C07 |
| ES 97 | M00055420D:G04 | ES 124 | M00042764B:B04 |
| ES 97 | M00055421B:D04 | ES 124 | M00042767D:D02 |
| ES 97 | M00055421C:C11 | ES 124 | M00042770B:B12 |
| ES 97 | M00055423A:A10 | ES 124 | M00042771B:A03 |
| ES 97 | M00055423A:G08 | ES 124 | M00042777A:D06 |
| ES 97 | M00055423C:H10 | ES 124 | M00042781C:A06 |
| ES 97 | M00055425D:C05 | ES 124 | M00042783C:A03 |
| ES 97 | M00055472A:F02 | ES 124 | M00042787C:E09 |
| ES 97 | M00055472B:H03 | ES 124 | M00042792C:G10 |
| ES 97 | M00055475D:G08 | ES 124 | M00042792D:F04 |
| ES 97 | M00055479A:G02 | ES 124 | M00042793B:G06 |
| ES 97 | M00055479C:C12 | ES 124 | M00042800A:A03 |
| ES 97 | M00055480C:H10 | ES 124 | M00042801B:B06 |
| ES 97 | M00055482D:A01 | ES 124 | M00042802C:G11 |
| ES 97 | M00055484A:G07 | ES 124 | M00042805A:E06 |
| ES 97 | M00055485A:C09 | ES 124 | M00042805D:H08 |
| ES 97 | M00055487B:F06 | ES 124 | M00042814D:B11 |
| ES 97 | M00001340A:E01 | ES 124 | M00042816B:F04 |
| ES 97 | M00001470C:G01 | ES 124 | M00042818A:E12 |
| ES 97 | M00001470C:G01 | ES 124 | M00042823B:G04 |
| ES 97 | M00001491B:C08 | ES 124 | M00042826B:C05 |
| ES 97 | M00001537D:F10 | ES 124 | M00042826D:C03 |
| ES 97 | M00001537D:F10 | ES 124 | M00042833D:G01 |
| ES 97 | M00001561B:G01 | ES 125 | M00042834C:B06 |
| ES 97 | M00001625A:B08 | ES 125 | M00042835C:C01 |
| ES 97 | M00001637A:D09 | ES 125 | M00042835D:D02 |
| ES 97 | M00003792B:A11 | ES 125 | M00042838D:E11 |
| ES 97 | M00003794C:D07 | ES 125 | M00042842A:C01 |
| ES 97 | M00003804D:A09 | ES 125 | M00042842D:E08 |
| ES 97 | M00003922B:H03 | ES 125 | M00042844C:C12 |
| ES 97 | M00003948A:B12 | ES 125 | M00042846C:D09 |
| ES 97 | M00003986D:G12 | ES 125 | M00042848D:G12 |
| ES 97 | M00003986D:G12 | ES 125 | M00042849D:F11 |
| ES 97 | M00004054C:G05 | ES 125 | M00042850B:C04 |
| ES 97 | M00004066C:D02 | ES 125 | M00042850C:C10 |
| ES 97 | M00004080A:A05 | ES 125 | M00042853A:G03 |
| ES 97 | M00004087D:B11 | ES 125 | M00042853D:A04 |
| ES 97 | M00004093D:C10 | ES 125 | M00042855A:B09 |
| ES 97 | M00004167C:D11 | ES 125 | M00042856C:F07 |
| ES 97 | M00004167C:D11 | ES 125 | M00042864A:E05 |
| ES 97 | M00004198B:A11 | ES 125 | M00042867D:H01 |
| ES 97 | M00004296D:G11 | ES 125 | M00042869C:E06 |
| ES 98 | M00004304A:D07 | ES 125 | M00042875C:E04 |
| ES 98 | M00004842C:B07 | ES 125 | M00042879B:F09 |
| ES 98 | M00004850C:G05 | ES 125 | M00056346C:C12 |
| ES 98 | M00004852D:A04 | ES 125 | M00056351B:D06 |
| ES 98 | M00004868B:D12 | ES 125 | M00056356B:F04 |
| ES 98 | M00004869D:D06 | ES 125 | M00056359C:A11 |
| ES 98 | M00004971B:G04 | ES 125 | M00056362D:G05 |
| ES 98 | M00004972C:E01 | ES 125 | M00056363A:B06 |
| ES 98 | M00005000B:H08 | ES 125 | M00056368C:E04 |
| ES 98 | M00005019D:D02 | ES 125 | M00056369B:D12 |
| ES 98 | M00005293B:D06 | ES 125 | M00056370B:G02 |
| ES 98 | M00005309D:E05 | ES 125 | M00056371B:F12 |
| ES 98 | M00005312A:D10 | ES 125 | M00056372B:C10 |
| ES 98 | M00005313C:B02 | ES 125 | M00056374B:H02 |
| ES 98 | M00005359A:A06 | ES 125 | M00056382C:H02 |
| ES 98 | M00005373D:H06 | ES 125 | M00056383A:C10 |
| ES 98 | M00005385C:A10 | ES 125 | M00056410B:E04 |
| ES 98 | M00005389C:C11 | ES 125 | M00056411C:E04 |
| ES 98 | M00005395A:D09 | ES 125 | M00056414B:A05 |
| ES 98 | M00005406C:A11 | ES 125 | M00056416C:B12 |
| ES 98 | M00005407B:E12 | ES 125 | M00056420C:D07 |
| ES 98 | M00005411C:C07 | ES 125 | M00056421A:F12 |
| ES 98 | M00005413B:F03 | ES 125 | M00056424A:F12 |
| ES 98 | M00005415C:F12 | ES 125 | M00056424B:H06 |
| ES 98 | M00005420B:C01 | ES 125 | M00056424D:A10 |
| ES 98 | M00005438B:A06 | ES 125 | M00056425A:H08 |
| ES 98 | M00005445C:A02 | ES 125 | M00056425D:B03 |
| ES 98 | M00005447C:D01 | ES 125 | M00056434A:C08 |
| ES 98 | M00005449C:E10 | ES 125 | M00056434D:E07 |
| ES 98 | M00005454B:C03 | ES 126 | M00056480B:C12 |
| ES 98 | M00005459B:B01 | ES 126 | M00056480D:A10 |
| ES 98 | M00005469B:A07 | ES 126 | M00056481A:F02 |
| ES 98 | M00005481D:C06 | ES 126 | M00056483D:F06 |
| ES 98 | M00005485B:B05 | ES 126 | M00056484B:G02 |
| ES 98 | M00005485D:A09 | ES 126 | M00056485B:B12 |
| ES 98 | M00005491B:H12 | ES 126 | M00056490D:E02 |
| ES 98 | M00005491D:B03 | ES 126 | M00056491D:G08 |
| ES 98 | M00005500B:E03 | ES 126 | M00056496B:A01 |
| ES 98 | M00005501B:E05 | ES 126 | M00056496C:H09 |
| ES 98 | M00005501D:G09 | ES 126 | M00056499C:F05 |
| ES 98 | M00005513B:F09 | ES 126 | M00056501C:H07 |
| ES 98 | M00005514C:A06 | ES 126 | M00056503B:G11 |
| ES 98 | M00005515B:H04 | ES 126 | M00056503B:G11 |
| ES 98 | M00005516D:H06 | ES 126 | M00056505B:H02 |
| ES 98 | M00005517B:F04 | ES 126 | M00056505D:D07 |
| ES 98 | M00005520C:E12 | ES 126 | M00056506C:G12 |
| ES 98 | M00005530C:A07 | ES 126 | M00056507D:B10 |
| ES 98 | M00005545B:A08 | ES 126 | M00056508B:B10 |
| ES 99 | M00005546A:G02 | ES 126 | M00056511A:H12 |
| ES 99 | M00005548A:A02 | ES 126 | M00056512B:C06 |
| ES 99 | M00005563C:B12 | ES 126 | M00056512C:E09 |
| ES 99 | M00005565A:F05 | ES 126 | M00056512D:C12 |
| ES 99 | M00005568C:B09 | ES 126 | M00056514B:E08 |
| ES 99 | M00007926A:A07 | ES 126 | M00056514C:G01 |
| ES 99 | M00007926D:A05 | ES 126 | M00056515C:C05 |
| ES 99 | M00007927C:C01 | ES 126 | M00056517B:G03 |
| ES 99 | M00007931A:A10 | ES 126 | M00056519C:H01 |
| ES 99 | M00007935D:A05 | ES 126 | M00056526C:E11 |
| ES 99 | M00007936D:B09 | ES 126 | M00056529D:F12 |
| ES 99 | M00007936D:B09 | ES 126 | M00056529D:H09 |
| ES 99 | M00007939B:A03 | ES 126 | M00056530A:D01 |
| ES 99 | M00007943A:C02 | ES 126 | M00056532B:G06 |
| ES 99 | M00007951C:A05 | ES 126 | M00056534A:D11 |
| ES 99 | M00007953B:A01 | ES 126 | M00056537B:H05 |
| ES 99 | M00007953D:H09 | ES 126 | M00056537C:A09 |
| ES 99 | M00007954C:B04 | ES 126 | M00056541B:A08 |
| ES 99 | M00007961B:F05 | ES 126 | M00056547A:C04 |
| ES 99 | M00007964B:G01 | ES 126 | M00056548A:C11 |
| ES 99 | M00007965A:G10 | ES 126 | M00056551A:F02 |
| ES 99 | M00007965B:C03 | ES 126 | M00056552A:A10 |
| ES 99 | M00007965B:C03 | ES 126 | M00056552D:B10 |
| ES 99 | M00007981D:B04 | ES 126 | M00056555A:F09 |
| ES 99 | M00007982A:F11 | ES 126 | M00056556C:G01 |
| ES 99 | M00007983B:D03 | ES 126 | M00056557C:D02 |
| ES 99 | M00007983D:H06 | ES 126 | M00056561C:D08 |
| ES 99 | M00007990D:D03 | ES 126 | M00056564C:E09 |
| ES 99 | M00007991D:G01 | ES 126 | M00056566C:H01 |
| ES 99 | M00007992D:G08 | ES 127 | M00056574B:A07 |
| ES 99 | M00007994A:C11 | ES 127 | M00056580B:F10 |
| ES 99 | M00007994D:A05 | ES 127 | M00056591C:E03 |
| ES 99 | M00007998C:F07 | ES 127 | M00056592A:F04 |
| ES 99 | M00005589C:F07 | ES 127 | M00056592C:C03 |
| ES 99 | M00005610D:B11 | ES 127 | M00056592D:D07 |
| ES 99 | M00005619B:A09 | ES 127 | M00056592D:D07 |
| ES 99 | M00005621A:H08 | ES 127 | M00056593B:E05 |
| ES 99 | M00005627B:B10 | ES 127 | M00056594C:C06 |
| ES 99 | M00005628B:C10 | ES 127 | M00056594C:C10 |
| ES 99 | M00005632A:H02 | ES 127 | M00056595A:A02 |
| ES 99 | M00005650C:A06 | ES 127 | M00056595A:C07 |
| ES 99 | M00005650C:D04 | ES 127 | M00056595B:F02 |
| ES 99 | M00005655B:F08 | ES 127 | M00056596A:E02 |
| ES 99 | M00005675A:G02 | ES 127 | M00056596C:E06 |
| ES 99 | M00005685D:D12 | ES 127 | M00056596C:H08 |
| ES 99 | M00005704C:D10 | ES 127 | M00056597A:F07 |
| ES 99 | M00005708B:B07 | ES 127 | M00056597D:C02 |
| ES 100 | M00042455C:D11 | ES 127 | M00056599D:D11 |
| ES 100 | M00054826A:B05 | ES 127 | M00056600D:H07 |
| ES 100 | M00055281A:E08 | ES 127 | M00056603C:D01 |
| ES 100 | M00005657D:A12 | ES 127 | M00056608C:E04 |
| ES 100 | M00005710A:D01 | ES 127 | M00056610B:H12 |
| ES 100 | M00005765D:F07 | ES 127 | M00056613A:A05 |
| ES 100 | M00005766C:F10 | ES 127 | M00056616B:C08 |
| ES 100 | M00005769B:A03 | ES 127 | M00056616D:A10 |
| ES 100 | M00005782A:B10 | ES 127 | M00056617B:H06 |
| ES 100 | M00005800D:D11 | ES 127 | M00056618A:B02 |
| ES 100 | M00005802B:H10 | ES 127 | M00056618B:F06 |
| ES 100 | M00005810B:F07 | ES 127 | M00056618D:F11 |
| ES 100 | M00005810B:G10 | ES 127 | M00056620D:E12 |
| ES 100 | M00005813B:E10 | ES 127 | M00056622D:C03 |
| ES 100 | M00005818D:B09 | ES 127 | M00056624D:H05 |
| ES 100 | M00005819C:B11 | ES 127 | M00056628C:F01 |
| ES 100 | M00005826D:G10 | ES 127 | M00056631B:G05 |
| ES 100 | M00005830C:D10 | ES 127 | M00056631D:C08 |
| ES 100 | M00006576A:B04 | ES 127 | M00056631D:D03 |
| ES 100 | M00006583B:H03 | ES 127 | M00056633B:B07 |
| ES 100 | M00006587B:A08 | ES 127 | M00056635A:A11 |
| ES 100 | M00006588A:H06 | ES 127 | M00056635A:E09 |
| ES 100 | M00006590A:C10 | ES 127 | M00056638A:D08 |
| ES 100 | M00006610C:D08 | ES 127 | M00056638B:B01 |
| ES 100 | M00006630D:C11 | ES 127 | M00056639A:E02 |
| ES 100 | M00006638A:G02 | ES 127 | M00056643D:G06 |
| ES 100 | M00006641C:H03 | ES 127 | M00056645C:B11 |
| ES 100 | M00006641C:H03 | ES 127 | M00056645D:F06 |
| ES 100 | M00006648B:A05 | ES 127 | M00056646C:C02 |
| ES 100 | M00006649D:B11 | ES 127 | M00056646D:G05 |
| ES 100 | M00006650A:A03 | ES 128 | M00056652D:F04 |
| ES 100 | M00006650D:D05 | ES 128 | M00056656C:H03 |
| ES 100 | M00006664A:B09 | ES 128 | M00056659C:G08 |
| ES 100 | M00006679D:C04 | ES 128 | M00056661B:A09 |
| ES 100 | M00006686B:B07 | ES 128 | M00056661D:E05 |
| ES 100 | M00006695D:H08 | ES 128 | M00056662B:F03 |
| ES 100 | M00006695D:H08 | ES 128 | M00056664B:G06 |
| ES 100 | M00006704C:G06 | ES 128 | M00056664C:B07 |
| ES 100 | M00006705B:A09 | ES 128 | M00056665B:A11 |
| ES 100 | M00006705C:G09 | ES 128 | M00056665C:E05 |
| ES 100 | M00006712C:F02 | ES 128 | M00056666A:C08 |
| ES 100 | M00006719A:E12 | ES 128 | M00056669B:G07 |
| ES 100 | M00006719A:H07 | ES 128 | M00056670A:A11 |
| ES 100 | M00006731B:B02 | ES 128 | M00056673D:E06 |
| ES 100 | M00006731B:C08 | ES 128 | M00056674B:E05 |
| ES 100 | M00006731B:D03 | ES 128 | M00056674D:H04 |
| ES 100 | M00006731C:E01 | ES 128 | M00056682D:F10 |
| ES 100 | M00006734C:A08 | ES 128 | M00056683C:B09 |
| ES 100 | M00006737D:A11 | ES 128 | M00056684D:A05 |
| ES 100 | M00006740B:G01 | ES 128 | M00056684D:F11 |
| ES 100 | M00006745C:A02 | ES 128 | M00056688B:F05 |
| ES 100 | M00006745D:D02 | ES 128 | M00056690C:F09 |
| ES 101 | M00006746C:B06 | ES 128 | M00056693C:C08 |
| ES 101 | M00006746C:B06 | ES 128 | M00056695A:H09 |
| ES 101 | M00006755D:A04 | ES 128 | M00056697C:E03 |
| ES 101 | M00006756B:F08 | ES 128 | M00056698C:E12 |
| ES 101 | M00006761C:C05 | ES 128 | M00056701B:E08 |
| ES 101 | M00006761C:D09 | ES 128 | M00056703A:D06 |
| ES 101 | M00006783B:F07 | ES 128 | M00056705D:E07 |
| ES 101 | M00006789D:A11 | ES 128 | M00056707B:E02 |
| ES 101 | M00006795D:A03 | ES 128 | M00056707D:D05 |
| ES 101 | M00006795D:D07 | ES 128 | M00056708C:C06 |
| ES 101 | M00006803A:C07 | ES 128 | M00056708D:D11 |
| ES 101 | M00006806B:C09 | ES 128 | M00056709A:A05 |
| ES 101 | M00006807A:G12 | ES 128 | M00056710A:C01 |
| ES 101 | M00006810A:D11 | ES 128 | M00056710B:F05 |
| ES 101 | M00006811D:D12 | ES 128 | M00056710B:H09 |
| ES 101 | M00006819B:B05 | ES 128 | M00056710D:F07 |
| ES 101 | M00006821D:B01 | ES 128 | M00056711A:C01 |
| ES 101 | M00006822C:A09 | ES 128 | M00056711A:F05 |
| ES 101 | M00006822D:E09 | ES 128 | M00056711A:F05 |
| ES 101 | M00006831D:H05 | ES 128 | M00056711D:A05 |
| ES 101 | M00006846A:D03 | ES 128 | M00056712C:A07 |
| ES 101 | M00006852A:C07 | ES 128 | M00056712C:B06 |
| ES 101 | M00006859B:D04 | ES 128 | M00056713D:G08 |
| ES 101 | M00006861B:A08 | ES 128 | M00056714C:H06 |
| ES 101 | M00006867C:F12 | ES 128 | M00056715A:D10 |
| ES 101 | M00006871D:F01 | ES 128 | M00056715A:E04 |
| ES 101 | M00006873C:E12 | ES 129 | M00056715A:G01 |
| ES 101 | M00006873D:B01 | ES 129 | M00056715B:C01 |
| ES 101 | M00006885C:G11 | ES 129 | M00056715D:C04 |
| ES 101 | M00006888A:G05 | ES 129 | M00056715D:E08 |
| ES 101 | M00006892B:F09 | ES 129 | M00056717B:C04 |
| ES 101 | M00006894D:A03 | ES 129 | M00056718C:B01 |
| ES 101 | M00006917B:A05 | ES 129 | M00056718C:G02 |
| ES 101 | M00006917C:A04 | ES 129 | M00056719A:D06 |
| ES 101 | M00006917D:D08 | ES 129 | M00056719A:F12 |
| ES 101 | M00006921A:H08 | ES 129 | M00056719B:A09 |
| ES 101 | M00006923B:H05 | ES 129 | M00056721A:F07 |
| ES 101 | M00006928A:A04 | ES 129 | M00056722A:G01 |
| ES 101 | M00006928B:D01 | ES 129 | M00056723B:D10 |
| ES 101 | M00006928B:D01 | ES 129 | M00056723C:C11 |
| ES 101 | M00006937D:F03 | ES 129 | M00056724D:E11 |
| ES 101 | M00006964A:A11 | ES 129 | M00056726C:G05 |
| ES 101 | M00006967B:B05 | ES 129 | M00056728A:H05 |
| ES 101 | M00006976B:D05 | ES 129 | M00056728B:D05 |
| ES 101 | M00006979D:B10 | ES 129 | M00056729B:D04 |
| ES 101 | M00006987A:G11 | ES 129 | M00056729C:H12 |
| ES 101 | M00006989C:E04 | ES 129 | M00056733C:D09 |
| ES 101 | M00006990D:E02 | ES 129 | M00056735D:B08 |
| ES 102 | M00006996C:F10 | ES 129 | M00056737B:G07 |
| ES 102 | M00006997A:A03 | ES 129 | M00056739A:D11 |
| ES 102 | M00007006B:A01 | ES 129 | M00056739B:D08 |
| ES 102 | M00007007B:H05 | ES 129 | M00056740C:B05 |
| ES 102 | M00007028C:C04 | ES 129 | M00056741B:C06 |
| ES 102 | M00007028C:C04 | ES 129 | M00056746D:A02 |
| ES 102 | M00007032C:F09 | ES 129 | M00056746D:D06 |
| ES 102 | M00007034B:B06 | ES 129 | M00056747A:D05 |
| ES 102 | M00007035C:E06 | ES 129 | M00056752A:E01 |
| ES 102 | M00007065C:F11 | ES 129 | M00056753D:A10 |
| ES 102 | M00007084B:G04 | ES 129 | M00056754A:A04 |
| ES 102 | M00007092D:F03 | ES 129 | M00056754B:D09 |
| ES 102 | M00007096A:E02 | ES 129 | M00056754B:H04 |
| ES 102 | M00007096C:E01 | ES 129 | M00056754D:A05 |
| ES 102 | M00007096D:H02 | ES 129 | M00056756B:A05 |
| ES 102 | M00007097A:B04 | ES 129 | M00056756D:B08 |
| ES 102 | M00007097D:D07 | ES 129 | M00056757B:F03 |
| ES 102 | M00007098A:C05 | ES 129 | M00056758B:C05 |
| ES 102 | M00007105D:C12 | ES 129 | M00056759A:F11 |
| ES 102 | M00007108A:D01 | ES 129 | M00056759B:G03 |
| ES 102 | M00007110C:F03 | ES 129 | M00056760D:A04 |
| ES 102 | M00007112A:A12 | ES 129 | M00056761A:F05 |
| ES 102 | M00007117D:H03 | ES 129 | M00056762C:E05 |
| ES 102 | M00007121C:G08 | ES 129 | M00056763C:D05 |
| ES 102 | M00007128B:G06 | ES 129 | M00056764A:E08 |
| ES 102 | M00007129A:F08 | ES 129 | M00056765A:A10 |
| ES 102 | M00007131C:A01 | ES 130 | M00056765C:E12 |
| ES 102 | M00007135D:B11 | ES 130 | M00056765D:D10 |
| ES 102 | M00007135D:B11 | ES 130 | M00056766B:A10 |
| ES 102 | M00007136C:C05 | ES 130 | M00056771C:F12 |
| ES 102 | M00007146D:F11 | ES 130 | M00056771D:C12 |
| ES 102 | M00007151A:B11 | ES 130 | M00056772D:A04 |
| ES 102 | M00007156A:E06 | ES 130 | M00056772D:A04 |
| ES 102 | M00007156D:F08 | ES 130 | M00056772D:E08 |
| ES 102 | M00007166A:E06 | ES 130 | M00056773A:H11 |
| ES 102 | M00007172B:C03 | ES 130 | M00056774B:A02 |
| ES 102 | M00007174C:D06 | ES 130 | M00056775D:A07 |
| ES 102 | M00007177A:E11 | ES 130 | M00056775D:C01 |
| ES 102 | M00007178C:D03 | ES 130 | M00056775D:C08 |
| ES 102 | M00007192A:E06 | ES 130 | M00056776D:A06 |
| ES 102 | M00007194A:E06 | ES 130 | M00056776D:D09 |
| ES 102 | M00007194B:B04 | ES 130 | M00056777B:C03 |
| ES 102 | M00007204C:G12 | ES 130 | M00056777D:B02 |
| ES 102 | M00007204D:D01 | ES 130 | M00056777D:F07 |
| ES 102 | M00008014A:B01 | ES 130 | M00056779A:E12 |
| ES 102 | M00008015D:B10 | ES 130 | M00056779D:H10 |
| ES 102 | M00008015D:F08 | ES 130 | M00056779D:H10 |
| ES 102 | M00008020C:C03 | ES 130 | M00056780D:C02 |
| ES 103 | M00008023B:D12 | ES 130 | M00056780D:F09 |
| ES 103 | M00008043B:B11 | ES 130 | M00056781C:E12 |
| ES 103 | M00008059D:B08 | ES 130 | M00056782D:B06 |
| ES 103 | M00008065A:B05 | ES 130 | M00056783B:G11 |
| ES 103 | M00008065C:F02 | ES 130 | M00056784A:B05 |
| ES 103 | M00008075C:A12 | ES 130 | M00056785B:F08 |
| ES 103 | M00008076C:F02 | ES 130 | M00056789A:C04 |
| ES 103 | M00008079C:C03 | ES 130 | M00056789D:E10 |
| ES 103 | M00008085B:C09 | ES 130 | M00056791D:F12 |
| ES 103 | M00008089A:E09 | ES 130 | M00056793C:H07 |
| ES 103 | M00008098D:H01 | ES 130 | M00056796A:H05 |
| ES 103 | M00008099B:G08 | ES 130 | M00056799B:E11 |
| ES 103 | M00021620B:F10 | ES 130 | M00056802B:H01 |
| ES 103 | M00021626C:C04 | ES 130 | M00056802B:H01 |
| ES 103 | M00021626D:F04 | ES 130 | M00056804B:E06 |
| ES 103 | M00021628B:B11 | ES 130 | M00056805D:B09 |
| ES 103 | M00021628B:D07 | ES 130 | M00056808B:B12 |
| ES 103 | M00021649A:E12 | ES 130 | M00056811A:C04 |
| ES 103 | M00021654A:A04 | ES 130 | M00056812C:E08 |
| ES 103 | M00021654A:A04 | ES 130 | M00056815A:B01 |
| ES 103 | M00021655C:H02 | ES 130 | M00056816B:A10 |
| ES 103 | M00021670D:G05 | ES 130 | M00056817C:C03 |
| ES 103 | M00021681D:C02 | ES 130 | M00056821D:C09 |
| ES 103 | M00021681D:C02 | ES 130 | M00056822C:G11 |
| ES 103 | M00022189A:B03 | ES 130 | M00056823A:B05 |
| ES 103 | M00022216D:D06 | ES 130 | M00056823C:A07 |
| ES 103 | M00022221A:D06 | ES 131 | M00056824B:C10 |
| ES 103 | M00022221D:D06 | ES 131 | M00056824D:E01 |
| ES 103 | M00022231D:E12 | ES 131 | M00056826A:B12 |
| ES 103 | M00022234B:D05 | ES 131 | M00056830C:G02 |
| ES 103 | M00022235C:C11 | ES 131 | M00056833C:C01 |
| ES 103 | M00022236A:A02 | ES 131 | M00056839A:G01 |
| ES 103 | M00022246A:H08 | ES 131 | M00056839A:G02 |
| ES 103 | M00022251C:A09 | ES 131 | M00056839C:F01 |
| ES 103 | M00022253A:E03 | ES 131 | M00056840D:H09 |
| ES 103 | M00022259C:B07 | ES 131 | M00056841D:G09 |
| ES 103 | M00022273B:A09 | ES 131 | M00056842B:F12 |
| ES 103 | M00022279B:H04 | ES 131 | M00056842B:F12 |
| ES 103 | M00022280A:G11 | ES 131 | M00056843B:H09 |
| ES 103 | M00022370A:G02 | ES 131 | M00056844A:E07 |
| ES 103 | M00022411D:G12 | ES 131 | M00056844C:A10 |
| ES 103 | M00022415C:B06 | ES 131 | M00056848B:C07 |
| ES 103 | M00022420B:H03 | ES 131 | M00056850B:E11 |
| ES 103 | M00022430B:D10 | ES 131 | M00056850B:E11 |
| ES 103 | M00022430C:D04 | ES 131 | M00056857B:C09 |
| ES 103 | M00022440D:D01 | ES 131 | M00056858A:B03 |
| ES 103 | M00022444A:C11 | ES 131 | M00056858B:A12 |
| ES 103 | M00022452A:B07 | ES 131 | M00056859A:D12 |
| ES 104 | M00022453B:H04 | ES 131 | M00056860A:F12 |
| ES 104 | M00022453B:H04 | ES 131 | M00056863C:E03 |
| ES 104 | M00022457C:G05 | ES 131 | M00056864B:H09 |
| ES 104 | M00022465D:F05 | ES 131 | M00056866B:E05 |
| ES 104 | M00022468A:E12 | ES 131 | M00056868D:E09 |
| ES 104 | M00022468C:E10 | ES 131 | M00056870A:E10 |
| ES 104 | M00022470B:G01 | ES 131 | M00056872A:A06 |
| ES 104 | M00022470B:G01 | ES 131 | M00056873C:E06 |
| ES 104 | M00022472D:E11 | ES 131 | M00056874B:H06 |
| ES 104 | M00022473D:B06 | ES 131 | M00056874C:D05 |
| ES 104 | M00022496D:F04 | ES 131 | M00056874D:G01 |
| ES 104 | M00022508A:C02 | ES 131 | M00056879A:E05 |
| ES 104 | M00022509A:B06 | ES 131 | M00056879B:H11 |
| ES 104 | M00022516B:E09 | ES 131 | M00056879D:A02 |
| ES 104 | M00022517B:E03 | ES 131 | M00056880D:B04 |
| ES 104 | M00022528A:H12 | ES 131 | M00056883D:A07 |
| ES 104 | M00022533C:E06 | ES 131 | M00056884B:C06 |
| ES 104 | M00022537A:C11 | ES 131 | M00056885C:C06 |
| ES 104 | M00022550C:B04 | ES 131 | M00056886A:C11 |
| ES 104 | M00022559D:D09 | ES 131 | M00056887B:F08 |
| ES 104 | M00022561A:A06 | ES 131 | M00056892C:A01 |
| ES 104 | M00022565A:A05 | ES 131 | M00056893B:H06 |
| ES 104 | M00022565A:A05 | ES 131 | M00056894D:G06 |
| ES 104 | M00022569A:A07 | ES 131 | M00056895B:A07 |
| ES 104 | M00022571C:D11 | ES 131 | M00056896A:F05 |
| ES 104 | M00021854C:E07 | ES 131 | M00056896A:F10 |
| ES 104 | M00021864A:E07 | ES 132 | M00056898D:D04 |
| ES 104 | M00021869D:D01 | ES 132 | M00056901A:A06 |
| ES 104 | M00021886D:F06 | ES 132 | M00056902A:H12 |
| ES 104 | M00021911A:H03 | ES 132 | M00056909B:E11 |
| ES 104 | M00021915B:E10 | ES 132 | M00056909C:D09 |
| ES 104 | M00021925C:H10 | ES 132 | M00056911B:F02 |
| ES 104 | M00021947B:C06 | ES 132 | M00056913B:G10 |
| ES 104 | M00022010B:H01 | ES 132 | M00056914D:B09 |
| ES 104 | M00022013D:H05 | ES 132 | M00056916C:B02 |
| ES 104 | M00022015D:F11 | ES 132 | M00056916C:F04 |
| ES 104 | M00022015D:F11 | ES 132 | M00056921A:C07 |
| ES 104 | M00022025C:D02 | ES 132 | M00056923C:E09 |
| ES 104 | M00022049C:B07 | ES 132 | M00056924D:B06 |
| ES 104 | M00022050C:D04 | ES 132 | M00056925D:C07 |
| ES 104 | M00022052D:A08 | ES 132 | M00056939A:F08 |
| ES 104 | M00022058D:A01 | ES 132 | M00056939D:B02 |
| ES 104 | M00022060B:F09 | ES 132 | M00056941D:E02 |
| ES 104 | M00022106B:D04 | ES 132 | M00056945A:B11 |
| ES 104 | M00022123A:D05 | ES 132 | M00056947D:F09 |
| ES 104 | M00022129A:E12 | ES 132 | M00056949C:F06 |
| ES 104 | M00022132A:D10 | ES 132 | M00056951B:F09 |
| ES 104 | M00022132C:F04 | ES 132 | M00056952C:A06 |
| ES 105 | M00022137B:G04 | ES 132 | M00056952D:H04 |
| ES 105 | M00022143A:C10 | ES 132 | M00056953B:A06 |
| ES 105 | M00022143A:D01 | ES 132 | M00056955B:G09 |
| ES 105 | M00022143A:D01 | ES 132 | M00056956B:F01 |
| ES 105 | M00022148A:A06 | ES 132 | M00056960A:C05 |
| ES 105 | M00022149C:C01 | ES 132 | M00056961A:B08 |
| ES 105 | M00022149D:C06 | ES 132 | M00056961C:G12 |
| ES 105 | M00022151A:D11 | ES 132 | M00056964B:A02 |
| ES 105 | M00022151A:G05 | ES 132 | M00056966D:A11 |
| ES 105 | M00022163A:C08 | ES 132 | M00056967A:D02 |
| ES 105 | M00022598B:E12 | ES 132 | M00056967A:E07 |
| ES 105 | M00022598C:D05 | ES 132 | M00056969B:C08 |
| ES 105 | M00022617B:C02 | ES 132 | M00056969D:B01 |
| ES 105 | M00022624C:C02 | ES 132 | M00056972A:F05 |
| ES 105 | M00022641A:C10 | ES 132 | M00056973D:B08 |
| ES 105 | M00022641A:E06 | ES 132 | M00056974C:F04 |
| ES 105 | M00022641B:F02 | ES 132 | M00056976C:F10 |
| ES 105 | M00022645D:A05 | ES 132 | M00056977A:G03 |
| ES 105 | M00022645D:C07 | ES 132 | M00056985B:C05 |
| ES 105 | M00022651D:B04 | ES 132 | M00056986A:F11 |
| ES 105 | M00022651D:C01 | ES 132 | M00056986D:G01 |
| ES 105 | M00022655A:D10 | ES 132 | M00056990C:B09 |
| ES 105 | M00022656D:E11 | ES 132 | M00056990D:C11 |
| ES 105 | M00022660A:B04 | ES 132 | M00056993A:B06 |
| ES 105 | M00022667A:C05 | ES 132 | M00056993D:D03 |
| ES 105 | M00022667D:E11 | ES 132 | M00056994B:F07 |
| ES 105 | M00022681D:E06 | ES 133 | M00056994C:C03 |
| ES 105 | M00022697A:D12 | ES 133 | M00056996D:A12 |
| ES 105 | M00022702B:B04 | ES 133 | M00056997C:H09 |
| ES 105 | M00022716C:C06 | ES 133 | M00056998A:E08 |
| ES 105 | M00022716C:C06 | ES 133 | M00057002D:B05 |
| ES 105 | M00022719A:F12 | ES 133 | M00057002D:B06 |
| ES 105 | M00022720B:A11 | ES 133 | M00057003B:B09 |
| ES 105 | M00022720C:C09 | ES 133 | M00057005B:C01 |
| ES 105 | M00022724C:D04 | ES 133 | M00057005C:D03 |
| ES 105 | M00022738B:D06 | ES 133 | M00057007C:B12 |
| ES 105 | M00022741B:B11 | ES 133 | M00057008C:E09 |
| ES 105 | M00022745C:C07 | ES 133 | M00057011A:D03 |
| ES 105 | M00022750A:A07 | ES 133 | M00057013B:D01 |
| ES 105 | M00022750A:A07 | ES 133 | M00057015A:C12 |
| ES 105 | M00022791B:F11 | ES 133 | M00057019C:H02 |
| ES 105 | M00022813B:A08 | ES 133 | M00057023A:H09 |
| ES 105 | M00022820D:C06 | ES 133 | M00057024A:E02 |
| ES 105 | M00022823A:D03 | ES 133 | M00057024A:G05 |
| ES 105 | M00022828A:C06 | ES 133 | M00057024D:H08 |
| ES 105 | M00022829A:H06 | ES 133 | M00057025C:A08 |
| ES 105 | M00022829C:H10 | ES 133 | M00057027C:G06 |
| ES 105 | M00022831B:H07 | ES 133 | M00057028D:D09 |
| ES 106 | M00022831C:A09 | ES 133 | M00057029A:C12 |
| ES 106 | M00022831D:C04 | ES 133 | M00057029D:A06 |
| ES 106 | M00022834C:G01 | ES 133 | M00057033A:F09 |
| ES 106 | M00022836A:G03 | ES 133 | M00057035B:C09 |
| ES 106 | M00022853C:C11 | ES 133 | M00057041D:B11 |
| ES 106 | M00022861D:B10 | ES 133 | M00057044C:F06 |
| ES 106 | M00022872A:B05 | ES 133 | M00057047B:C02 |
| ES 106 | M00022876B:B05 | ES 133 | M00057049A:G06 |
| ES 106 | M00022876D:D08 | ES 133 | M00057049C:H05 |
| ES 106 | M00022880C:G09 | ES 133 | M00057052D:B11 |
| ES 106 | M00022892C:G07 | ES 133 | M00057052D:G09 |
| ES 106 | M00022895B:B11 | ES 133 | M00057055B:G08 |
| ES 106 | M00022897D:H03 | ES 133 | M00057055B:G08 |
| ES 106 | M00022898C:F04 | ES 133 | M00057058C:F09 |
| ES 106 | M00022899A:C09 | ES 133 | M00057059D:F06 |
| ES 106 | M00022901D:E11 | ES 133 | M00057059D:H09 |
| ES 106 | M00022901D:E11 | ES 133 | M00057060B:A12 |
| ES 106 | M00022902C:H10 | ES 133 | M00057061C:D04 |
| ES 106 | M00022908B:H03 | ES 133 | M00057063A:C08 |
| ES 106 | M00022911B:G01 | ES 133 | M00057065C:D04 |
| ES 106 | M00022928A:F03 | ES 133 | M00057066A:A04 |
| ES 106 | M00022934D:B03 | ES 133 | M00057070D:B08 |
| ES 106 | M00022956B:B09 | ES 133 | M00057072B:E02 |
| ES 106 | M00022961A:B11 | ES 133 | M00057073D:A05 |
| ES 106 | M00022973A:G07 | ES 133 | M00057074D:C09 |
| ES 106 | M00022973C:G08 | ES 133 | M00057074D:C09 |
| ES 106 | M00022974D:D10 | ES 134 | M00055909B:G01 |
| ES 106 | M00022995C:E02 | ES 134 | M00055909C:E08 |
| ES 106 | M00022997A:C08 | ES 134 | M00055911B:E06 |
| ES 106 | M00022998B:C08 | ES 134 | M00055912C:E10 |
| ES 106 | M00023002D:G10 | ES 134 | M00055912D:C05 |
| ES 106 | M00023015A:D10 | ES 134 | M00055913B:D05 |
| ES 106 | M00023015C:D02 | ES 134 | M00055919A:A06 |
| ES 106 | M00023020D:G09 | ES 134 | M00055921A:E03 |
| ES 106 | M00023023C:F03 | ES 134 | M00055921B:B11 |
| ES 106 | M00023029A:E06 | ES 134 | M00055922A:C02 |
| ES 106 | M00023331D:A11 | ES 134 | M00055924A:H11 |
| ES 106 | M00023347D:C12 | ES 134 | M00055930A:B08 |
| ES 106 | M00023377D:C09 | ES 134 | M00055931A:A03 |
| ES 106 | M00023393D:C02 | ES 134 | M00055931A:C01 |
| ES 106 | M00023393D:E12 | ES 134 | M00055931B:E01 |
| ES 106 | M00023399C:C08 | ES 134 | M00055936B:E07 |
| ES 106 | M00023409A:G08 | ES 134 | M00055937B:C02 |
| ES 106 | M00023414B:F03 | ES 134 | M00055941B:B12 |
| ES 106 | M00023428C:D03 | ES 134 | M00055941B:B12 |
| ES 106 | M00023428D:F11 | ES 134 | M00055945A:H11 |
| ES 106 | M00023430B:D10 | ES 134 | M00055945B:E10 |
| ES 106 | M00023518C:A04 | ES 134 | M00055946D:G07 |
| ES 107 | M00023520A:G07 | ES 134 | M00055951C:C02 |
| ES 107 | M00026804D:D03 | ES 134 | M00055956C:E02 |
| ES 107 | M00026805B:B04 | ES 134 | M00055958D:F02 |
| ES 107 | M00026848C:G11 | ES 134 | M00055959D:A12 |
| ES 107 | M00026854A:E07 | ES 134 | M00055966C:A03 |
| ES 107 | M00026856C:C11 | ES 134 | M00055966C:D06 |
| ES 107 | M00026860D:E01 | ES 134 | M00055971C:E07 |
| ES 107 | M00026861D:A09 | ES 134 | M00055973A:D04 |
| ES 107 | M00026865D:G11 | ES 134 | M00055976B:F01 |
| ES 107 | M00026866A:H08 | ES 134 | M00055979B:B09 |
| ES 107 | M00026873B:E11 | ES 134 | M00055980A:A10 |
| ES 107 | M00026873D:B08 | ES 134 | M00055981D:A07 |
| ES 107 | M00026879A:B02 | ES 134 | M00055984C:C02 |
| ES 107 | M00026879C:D10 | ES 134 | M00055985D:D01 |
| ES 107 | M00026890C:D02 | ES 134 | M00055990C:B05 |
| ES 107 | M00026893C:A01 | ES 134 | M00055992C:E11 |
| ES 107 | M00026896D:E10 | ES 134 | M00056139D:E04 |
| ES 107 | M00026899C:G11 | ES 134 | M00056139D:G01 |
| ES 107 | M00026899C:G11 | ES 134 | M00056140B:H07 |
| ES 107 | M00026900B:C02 | ES 134 | M00056140D:E07 |
| ES 107 | M00026902A:G04 | ES 134 | M00056141A:D05 |
| ES 107 | M00026906B:C10 | ES 134 | M00056141D:B09 |
| ES 107 | M00026909A:G03 | ES 134 | M00056143A:E09 |
| ES 107 | M00026917D:H03 | ES 134 | M00056144B:C09 |
| ES 107 | M00026926D:C08 | ES 134 | M00056145C:B04 |
| ES 107 | M00026934D:E09 | ES 134 | M00056149C:B01 |
| ES 107 | M00026936D:C12 | ES 135 | M00056150B:C12 |
| ES 107 | M00026937C:B08 | ES 135 | M00056153C:D01 |
| ES 107 | M00026938A:F04 | ES 135 | M00056156D:A12 |
| ES 107 | M00026938A:F04 | ES 135 | M00056160D:A08 |
| ES 107 | M00026949B:H10 | ES 135 | M00056161D:G04 |
| ES 107 | M00026950A:F12 | ES 135 | M00056162B:F08 |
| ES 107 | M00026950D:H01 | ES 135 | M00056162B:F08 |
| ES 107 | M00026951A:G06 | ES 135 | M00056162D:D06 |
| ES 107 | M00026951A:G11 | ES 135 | M00056162D:E09 |
| ES 107 | M00026951A:G11 | ES 135 | M00056167D:B08 |
| ES 107 | M00026951C:D03 | ES 135 | M00056169A:F06 |
| ES 107 | M00026975C:B03 | ES 135 | M00056171C:H11 |
| ES 107 | M00026977A:E09 | ES 135 | M00056171C:H12 |
| ES 107 | M00026984A:D10 | ES 135 | M00056180B:H09 |
| ES 107 | M00026985C:B05 | ES 135 | M00056184B:D08 |
| ES 107 | M00026986B:H10 | ES 135 | M00056184C:H03 |
| ES 107 | M00026993B:H06 | ES 135 | M00056184D:F01 |
| ES 107 | M00026994C:A07 | ES 135 | M00056185D:A03 |
| ES 107 | M00026996D:A06 | ES 135 | M00056185D:D06 |
| ES 107 | M00027000C:F05 | ES 135 | M00056186C:F02 |
| ES 107 | M00027006B:H01 | ES 135 | M00056190D:G02 |
| ES 107 | M00027013D:E10 | ES 135 | M00056192D:E04 |
| ES 108 | M00027014C:G04 | ES 135 | M00056192D:H02 |
| ES 108 | M00027014D:G04 | ES 135 | M00056195B:C08 |
| ES 108 | M00027016D:G06 | ES 135 | M00056198A:D07 |
| ES 108 | M00027021D:H11 | ES 135 | M00056199D:A09 |
| ES 108 | M00027028D:C07 | ES 135 | M00056201C:H08 |
| ES 108 | M00027030C:C08 | ES 135 | M00056203A:H10 |
| ES 108 | M00027034B:D09 | ES 135 | M00056204B:A04 |
| ES 108 | M00027034C:D11 | ES 135 | M00056205B:D01 |
| ES 108 | M00027035D&H09 | ES 135 | M00056206A:E06 |
| ES 108 | M00027039A:F06 | ES 136 | M00055997C:G11 |
| ES 108 | M00027039B:E09 | ES 136 | M00055999C:G10 |
| ES 108 | M00027042C:G11 | ES 136 | M00055999D:G06 |
| ES 108 | M00027046B:E05 | ES 136 | M00056000A:F12 |
| ES 108 | M00027051A:A07 | ES 136 | M00056000C:D09 |
| ES 108 | M00027054B:B03 | ES 136 | M00056001A:B06 |
| ES 108 | M00027076D:F07 | ES 136 | M00056001A:B07 |
| ES 108 | M00027084C:H10 | ES 136 | M00056001C:E09 |
| ES 108 | M00027088D:H06 | ES 136 | M00056003A:E06 |
| ES 108 | M00027090A:E08 | ES 136 | M00056005B:E05 |
| ES 108 | M00027093C:B08 | ES 136 | M00056005D:C04 |
| ES 108 | M00027096A:G07 | ES 136 | M00056007A:A11 |
| ES 108 | M00027097C:G11 | ES 136 | M00056007C:F06 |
| ES 108 | M00027111A:H04 | ES 136 | M00056016D:D06 |
| ES 108 | M00027134A:G02 | ES 136 | M00056018B:G05 |
| ES 108 | M00027139D:C06 | ES 136 | M00056020A:D10 |
| ES 108 | M00027140A:C11 | ES 136 | M00056020D:D07 |
| ES 108 | M00027163A:D11 | ES 136 | M00056028C:F03 |
| ES 108 | M00027165C:F11 | ES 136 | M00056036D:B06 |
| ES 108 | M00027168C:H10 | ES 136 | M00056037C:B02 |
| ES 108 | M00027171D:B07 | ES 136 | M00056038D:F04 |
| ES 108 | M00027172A:C03 | ES 136 | M00056041A:C04 |
| ES 108 | M00027173D:D08 | ES 136 | M00056042A:A01 |
| ES 108 | M00027183B:B01 | ES 136 | M00056045D:H01 |
| ES 108 | M00027193C:C05 | ES 136 | M00056050C:A03 |
| ES 108 | M00027194D:A05 | ES 136 | M00056053A:A09 |
| ES 108 | M00027197A:G07 | ES 136 | M00056053A:D12 |
| ES 108 | M00027197B:F07 | ES 136 | M00056055A:A07 |
| ES 108 | M00027203B:H08 | ES 136 | M00056055B:B01 |
| ES 108 | M00027207B:E09 | ES 136 | M00056055C:D03 |
| ES 108 | M00027217A:G03 | ES 136 | M00056058A:H04 |
| ES 108 | M00027220A:B12 | ES 136 | M00056060B:B10 |
| ES 108 | M00027222A:C09 | ES 136 | M00056061B:F06 |
| ES 108 | M00027229D:E06 | ES 136 | M00056066D:H07 |
| ES 108 | M00027231D:A03 | ES 136 | M00056067B:D08 |
| ES 108 | M00027524B:B11 | ES 136 | M00056074D:G10 |
| ES 108 | M00027527A:G04 | ES 136 | M00056077D:E06 |
| ES 108 | M00027532C:C02 | ES 136 | M00056077D:E12 |
| ES 108 | M00027535D:E08 | ES 136 | M00056077D:E12 |
| ES 109 | M00027536D:G12 | ES 136 | M00056079B:D12 |
| ES 109 | M00027543C:B09 | ES 136 | M00056079B:F07 |
| ES 109 | M00027543D:G07 | ES 136 | M00056079C:C11 |
| ES 109 | M00027556D:G10 | ES 136 | M00056081D:B05 |
| ES 109 | M00027561C:C04 | ES 136 | M00056081D:B09 |
| ES 109 | M00027562B:C02 | ES 136 | M00056082C:F06 |
| ES 109 | M00027564A:D03 | ES 136 | M00056085D:H11 |
| ES 109 | M00027571C:C11 | ES 136 | M00056094A:H07 |
| ES 109 | M00027573A:F09 | ES 136 | M00056098A:H01 |
| ES 109 | M00027578B:F05 | ES 136 | M00056099B:G09 |
| ES 109 | M00027578C:E04 | ES 136 | M00056099B:H11 |
| ES 109 | M00027580C:E10 | ES 136 | M00056099B:H11 |
| ES 109 | M00027581B:E01 | ES 136 | M00056103A:D12 |
| ES 109 | M00027588A:C01 | ES 136 | M00056103C:H12 |
| ES 109 | M00027588C:A06 | ES 136 | M00056107B:E06 |
| ES 109 | M00027594B:C03 | ES 136 | M00056108D:B12 |
| ES 109 | M00027604A:G10 | ES 136 | M00056108D:B12 |
| ES 109 | M00027604A:G10 | ES 136 | M00056110C:D09 |
| ES 109 | M00027605C:E05 | ES 136 | M00056111D:H02 |
| ES 109 | M00027607A:H05 | ES 136 | M00056112A:H02 |
| ES 109 | M00027608C:H07 | ES 136 | M00056114C:C06 |
| ES 109 | M00027616C:G12 | ES 136 | M00056125B:D09 |
| ES 109 | M00027628C:A01 | ES 136 | M00056128C:B10 |
| ES 109 | M00027639B:E11 | ES 136 | M00056131B:C12 |
| ES 109 | M00027641B:A01 | ES 136 | M00056133D:D09 |
| ES 109 | M00027652B:G03 | ES 136 | M00056136A:B11 |

TABLE 9

| | | | | Pri- | | | | | | | | | |
|---|---|---|---|---|---|---|---|---|---|---|---|---|---|
| | | | | mary | Hist- | | | | | | | | |
| | | Ana- | | Tu- | o- | | | Inci- | Reg- | | | Dist | |
| Pat- | Path | tom- | Primary | mor | path | | Lymph- | dence | ional | Distant | Descrip | Met | |
| ient | Report | ical | Tumor | Gr- | Gr- | Local | node | Lym- | Lym- | Met & | Distant | Gr- | |
| ID | ID | Loc | size | ade | ade | Invasion | Met | phn | ph | Loc | Met | ade | Comment |
| 15 | 21 | Ascend- | 4.0 | T3 | G2 | extending into | positive | 3/8 | N1 | negative | | MX | invasive |
| | | ing | | | | subserosal | | | | | | | adenocarcinoma, |
| | | colon | | | | adipose tissue | | | | | | | moderately |
| | | | | | | | | | | | | | differentiated; |
| | | | | | | | | | | | | | focal perincural |
| | | | | | | | | | | | | | invasion is seen |
| 52 | 71 | Ascend- | 9.0 | T3 | G3 | Invasion | negative | 0/12 | N0 | negative | | M0 | Hyperplastic |
| | | ing | | | | through | | | | | | | polyp in |
| | | colon | | | | muscularis | | | | | | | appendix. |
| | | | | | | propria, | | | | | | | |
| 121 | 140 | Sigmoid | 6 | T4 | G2 | Invasion of | negative | 0/34 | N0 | negative | | M0 | Perineural |
| | | | | | | muscularis | | | | | | | invasion; donut |
| | | | | | | propria into | | | | | | | anastomosis |
| | | | | | | serosa, | | | | | | | negative. One |
| | | | | | | involving | | | | | | | tubulovillous |
| 125 | 144 | Cecum | 6 | T3 | G2 | Invasion | negative | 0/19 | N0 | negative | | M0 | patient history |
| | | | | | | through the | | | | | | | of metastatic |
| | | | | | | muscularis | | | | | | | melanoma |
| | | | | | | propria into | | | | | | | |
| | | | | | | suserosal | | | | | | | |
| | | | | | | adipose tissue. | | | | | | | |
| | | | | | | Ileocecal | | | | | | | |
| | | | | | | junction. | | | | | | | |
| 128 | 147 | Trans- | 5.0 | T3 | G2 | Invasion of | positive | 1/5 | N1 | negative | | M0 | |
| | | verse | | | | museularis | | | | | | | |
| | | colon | | | | propria into | | | | | | | |
| | | | | | | percolonic fat | | | | | | | |
| 130 | 149 | Splenic | 5.5 | T3 | | through wall | positive | 10/24 | N2 | negative | | M1 | |
| | | flexure | | | | and into | | | | | | | |
| | | | | | | surrounding | | | | | | | |
| | | | | | | adipose tissue | | | | | | | |
| 133 | 152 | Rectum | 5.0 | T3 | G2 | Invasion | negative | 0/9 | N0 | negative | | M0 | Small separate |
| | | | | | | through | | | | | | | tubular |
| | | | | | | muscularis | | | | | | | adenoma (0.4 |
| | | | | | | propria into non | | | | | | | cm) |
| | | | | | | peritonealized | | | | | | | |
| | | | | | | pericolic tissue, | | | | | | | |
| | | | | | | gross | | | | | | | |
| | | | | | | configuration is | | | | | | | |
| | | | | | | annular. | | | | | | | |
| 141 | 160 | Cecrum | 5.5 | T3 | G2 | Invasion of | positive | 7/21 | N2 | positive | adenpcarcinoma | M1 | Perineural |
| | | | | | | muscularis | | | | (Liver) | consitant | | invasion |
| | | | | | | propria into | | | | | with | | identified |
| | | | | | | pericolonic | | | | | primary | | adjacent to |
| | | | | | | adipose tissue, | | | | | | | metastatic |
| | | | | | | but not through | | | | | | | adenocarcinoma |
| | | | | | | serosa. Arising | | | | | | | |
| | | | | | | from tubular | | | | | | | |
| | | | | | | adenoma. | | | | | | | |
| 156 | 175 | Hepatic | 3.8 | T3 | G2 | Invasion | positive | 2/13 | N1 | negative | | M0 | Separate |
| | | flexure | | | | through | | | | | | | tubolovillous |
| | | | | | | mucsularis | | | | | | | and tubular |
| | | | | | | propria into | | | | | | | adenomas |
| | | | | | | subserosa/peric | | | | | | | |
| | | | | | | olic adipose, no | | | | | | | |
| | | | | | | serosal | | | | | | | |
| | | | | | | involvement. | | | | | | | |
| | | | | | | Gross | | | | | | | |
| | | | | | | configuration | | | | | | | |
| | | | | | | annular. | | | | | | | |
| 228 | 247 | Rectum | 5.8 | T3 | G2 | Invasion | positive | 1/8 | N1 | negative | | MX | Hyperplastic |
| | | | | | to | through | | | | | | | polyps |
| | | | | | G3 | muscularis | | | | | | | |
| | | | | | | propria to | | | | | | | |
| | | | | | | involve | | | | | | | |
| | | | | | | subserosal, | | | | | | | |
| | | | | | | perirectoal | | | | | | | |
| | | | | | | adipose, and | | | | | | | |
| | | | | | | serosa | | | | | | | |
| 264 | 283 | Ascend- | 5.5 | T3 | G2 | Invasion | negative | 0/10 | N0 | negative | | M0 | Tubulovillous |
| | | ing | | | | through | | | | | | | adenoma with |
| | | colon | | | | muscularis | | | | | | | high grade |
| | | | | | | propria into | | | | | | | dysplasia |
| | | | | | | subserosal | | | | | | | |
| | | | | | | adipose tissue | | | | | | | |
| 266 | 285 | Trans- | 9 | T3 | G2 | Invades through | negative | 0/15 | N1 | positive | 0.4 cm, | MX | |
| | | verse | | | | muscularis | | | | (Mesen- | may | | |
| | | colon | | | | propria to | | | | teric | represent | | |
| | | | | | | involve | | | | deposit) | lymph node | | |
| | | | | | | pericolonic | | | | | completely | | |
| | | | | | | adipose, | | | | | replaced by | | |
| | | | | | | extends to | | | | | tumor | | |
| | | | | | | serosa. | | | | | | | |
| 268 | 287 | Cecum | 6.5 | T2 | G2 | Invades full | negative | 0/12 | N0 | negative | | M0 | |
| | | | | | | thickness of | | | | | | | |
| | | | | | | muscularis | | | | | | | |
| | | | | | | propria, but | | | | | | | |
| | | | | | | mesenteric | | | | | | | |
| | | | | | | adipose free of | | | | | | | |
| | | | | | | malignancy | | | | | | | |
| 278 | 297 | Rectum | 4 | T3 | G2 | Invasion into | positive | 7/10 | N2 | negative | | M0 | Descending |
| | | | | | | perirectal | | | | | | | colon polyps, |
| | | | | | | adipose tissue. | | | | | | | no HGD or |
| | | | | | | | | | | | | | carcinoma |
| | | | | | | | | | | | | | identified. |
| 295 | 314 | Ascend- | 5.0 | T3 | G2 | Invasion | negative | 0/12 | N0 | negative | | M0 | Melanosis coli |
| | | ing | | | | through | | | | | | | and diverticular |
| | | colon | | | | muscularis | | | | | | | disease. |
| | | | | | | propria into | | | | | | | |
| | | | | | | percolic adipose | | | | | | | |
| | | | | | | tissue. | | | | | | | |
| 339 | 358 | Recto- | 6 | T3 | G2 | Extends into | negative | 0/6 | N0 | negative | | M0 | 1 hyperplastic |
| | | sigmoid | | | | perirectal fat | | | | | | | polyp identified |
| | | | | | | but does not | | | | | | | |
| | | | | | | reach serosa | | | | | | | |
| 341 | 360 | Ascend- | 2 cm | T3 | G2 | Invasion | negative | 0/4 | N0 | negative | | MX | |
| | | ing | invasive | | | through | | | | | | | |
| | | colon | | | | muscularis | | | | | | | |
| | | | | | | propria to | | | | | | | |
| | | | | | | involve | | | | | | | |
| | | | | | | pericolonic fat. | | | | | | | |
| | | | | | | Arising from | | | | | | | |
| | | | | | | villous | | | | | | | |
| | | | | | | adenoma. | | | | | | | |
| 356 | 375 | Sigmoid | 6.5 | T3 | G2 | Through colon | negative | 0/4 | N0 | negative | | M0 | |
| | | | | | | wall into | | | | | | | |
| | | | | | | subserosal | | | | | | | |
| | | | | | | adipose tissue | | | | | | | |
| | | | | | | No serosal | | | | | | | |
| | | | | | | spread seen. | | | | | | | |
| 360 | 412 | Ascend- | 4.3 | T3 | G2 | Invasion thru | positive | 1/5 | N1 | negative | | M0 | Two mucosal |
| | | ing | | | | muscularis | | | | | | | polyps |
| | | colon | | | | propria to | | | | | | | |
| | | | | | | pericolonic fat | | | | | | | |
| 392 | 444 | Ascend- | 2 | T3 | G2 | Invasion | positive | 1/6 | N1 | positive | Macrovesicular | M1 | Tumor arising |
| | | ing | | | | through | | | | (Liver) | and | | at prior |
| | | colon | | | | muscularis | | | | | microvesicular | | ileocolic |
| | | | | | | propria into | | | | | steatosis | | surgical |
| | | | | | | subserosal | | | | | | | anastomosis. |
| | | | | | | adipose tissue, | | | | | | | |
| | | | | | | not serosa. | | | | | | | |
| 393 | 445 | Cecum | 6.0 | T3 | G2 | Cecum, invades | negative | 0/21 | N0 | negative | | M0 | |
| | | | | | | through | | | | | | | |
| | | | | | | muscularis | | | | | | | |
| | | | | | | propria to | | | | | | | |
| | | | | | | involve | | | | | | | |
| | | | | | | subserosal | | | | | | | |
| | | | | | | adipose tissue | | | | | | | |
| | | | | | | but not serosa. | | | | | | | |
| 413 | 465 | Ascend- | 4.8 | T3 | G2 | Invasive | negative | 0/7 | N0 | positive | ademocarcinoma | M1 | rediagnosis of |
| | | ing | | | | through | | | | (Liver) | in | | oophorectomy |
| | | colon | | | | muscularis to | | | | | multiple | | path to |
| | | | | | | involve | | | | | slides | | metastatic |
| | | | | | | periserosal fat, | | | | | | | colon cancer. |
| | | | | | | abutting | | | | | | | |
| | | | | | | ileocccal | | | | | | | |
| | | | | | | junction | | | | | | | |
| 505 | 383 | | 7.5 cm | T3 | G2 | Invasion | positive | 2/17 | N1 | positive | moderately | M1 | Anatomical |
| | | | max | | | through | | | | (Liver) | differentiated | | location of |
| | | | dim | | | muscularis | | | | | adenocarcinoma, | | primary not |
| | | | | | | propria | | | | | consistant | | notated in |
| | | | | | | involving | | | | | with | | report. |
| | | | | | | pericolic | | | | | primary | | Evidence of |
| | | | | | | adipose, serosal | | | | | | | chronic colitis. |
| | | | | | | surface | | | | | | | |
| | | | | | | uninvolved | | | | | | | |
| 517 | 395 | Sigmoid | 3 | T3 | G2 | penetrates | positive | 6/6 | N2 | negative | | M0 | No mention of |
| | | | | | | muscularis | | | | | | | distant met in |
| | | | | | | propria, | | | | | | | report |
| | | | | | | involves | | | | | | | |
| | | | | | | pericolonic fat | | | | | | | |
| 534 | 553 | Ascend- | 12 | T3 | G3 | Invasion | negative | 0/8 | N0 | negative | | M0 | Omentum with |
| | | ing | | | | through the | | | | | | | fibrosis and fat |
| | | colon | | | | muscularis | | | | | | | necrosis. Small |
| | | | | | | propria | | | | | | | bowel with |
| | | | | | | involving | | | | | | | acute and |
| | | | | | | pericolic fat | | | | | | | chronic |
| | | | | | | Serosa free | | | | | | | serositis, focal |
| | | | | | | of tumor | | | | | | | abscess and |
| | | | | | | | | | | | | | adhesions |
| 546 | 565 | Ascend- | 5.5 | T3 | G2 | Invasion | positive | 6/12 | N2 | positive | metastatic | M1 | |
| | | ing | | | | through | | | | (Liver) | adenocarcinoma | | |
| | | colon | | | | muscularis | | | | | | | |
| | | | | | | propria | | | | | | | |
| | | | | | | extensively | | | | | | | |
| | | | | | | through | | | | | | | |
| | | | | | | submucosal and | | | | | | | |
| | | | | | | extending to | | | | | | | |
| | | | | | | serosa. | | | | | | | |
| 577 | 596 | Cecum | 11.5 | T3 | G2 | Invasion | negative | 0/58 | N0 | negative | | M0 | Appendix |
| | | | | | | through the | | | | | | | dilated/ and |
| | | | | | | bowel wall, into | | | | | | | fibrotic, but not |
| | | | | | | suberosal | | | | | | | involved by |
| | | | | | | adipose. | | | | | | | tumor |
| | | | | | | Serosal surface | | | | | | | |
| | | | | | | free of tumor | | | | | | | |
| 695 | 714 | Cecum | 14 | T3 | G2 | extending | negative | 022 | N0 | negative | | MX | tubular |
| | | | | | | through bowel | | | | | | | adenoma and |
| | | | | | | wall into | | | | | | | hyperplastic |
| | | | | | | scrosal fat | | | | | | | polyps present, |
| | | | | | | | | | | | | | moderately |
| | | | | | | | | | | | | | differentiated |
| | | | | | | | | | | | | | adenoma with |
| | | | | | | | | | | | | | mucinous |
| | | | | | | | | | | | | | diferentiation |
| | | | | | | | | | | | | | (% not stated) |
| 784 | 803 | Ascend- | 3.5 | T3 | G3 | through | positive | 5/17 | N2 | positive | | M1 | invasive poorly |
| | | ing | | | | muscularis | | | | (Liver) | | | differentiated |
| | | colon | | | | propria into | | | | | | | adenosquamous |
| | | | | | | pericolic soft | | | | | | | carcinoma |
| | | | | | | tissues | | | | | | | |
| 786 | 805 | De- | 9.5 | T3 | G2 | through | negative | 0/12 | N0 | positive | | M1 | moderately |
| | | scend- | | | | muscularis | | | | (Liver) | | | differentiated |
| | | ing | | | | propria into | | | | | | | invasive |
| | | colon | | | | pericolic fat, | | | | | | | adenocarcinoma |
| | | | | | | but not at | | | | | | | |
| | | | | | | serosal surface | | | | | | | |
| 791 | 810 | Ascend- | 5.8 | T3 | G3 | through the | positive | 13/25 | N2 | positive | | M1 | poorly |
| | | ing | | | | muscularis | | | | (Liver) | | | differentiated |
| | | colon | | | | propria into | | | | | | | invasive |
| | | | | | | pericolic fat | | | | | | | colonic |
| | | | | | | | | | | | | | adenocarcinoma |
| 888 | 908 | Ascend- | 2.0 | T2 | G1 | into muscularis | positive | 3/21 | N0 | positive | | M1 | well- to |
| | | ing | | | | propria | | | | (Liver) | | | moderately- |
| | | colon | | | | | | | | | | | differentiated |
| | | | | | | | | | | | | | adenocarcinoma; |
| | | | | | | | | | | | | | this patient |
| | | | | | | | | | | | | | has tumors of |
| | | | | | | | | | | | | | the ascending |
| | | | | | | | | | | | | | colon and the |
| | | | | | | | | | | | | | sigmoid colon |
| 889 | 909 | Cecum | 4.8 | T3 | G2 | through | positive | 1/4 | N1 | positive | | M1 | moderately |
| | | | | | | muscularis | | | | (Liver) | | | differentiated |
| | | | | | | propria int | | | | | | | adenocarcinoma |

TABLE 10

| | % Pts | % Pts | % Pts | | | | |
|---|---|---|---|---|---|---|---|
| SEQ ID | > = 2× | > = 2_5× | > = 5× | | | | |
| NO | T/N | T/N | T/N | P15 | P52 | P121 | P125 |
| 18 | 30.3 | 15.2 | 3.0 | 1.855 | 2.705 | 1.000 | 2.280 |
| 22 | 45.5 | 39.4 | 18.2 | 2.196 | 1.719 | 0.604 | 2.388 |
| 127 | 27.3 | 18.2 | 6.1 | 1.000 | 1.620 | 1.822 | 1.692 |
| 139 | 21.2 | 18.2 | 15.2 | 1000.000 | 0.001 | 2.345 | 1.000 |
| 148 | 27.3 | 18.2 | 6.1 | 1.000 | 1.620 | 1.822 | 1.692 |
| 155 | 45.5 | 12.1 | 3.0 | 1.870 | 3.104 | 1.361 | 2.388 |
| 246 | 42.4 | 9.1 | 0.0 | 2.211 | 2.347 | 1.000 | 1.493 |
| 272 | 48.5 | 27.3 | 12.1 | 1.735 | 3.110 | 1.379 | 2.277 |
| 273 | 21.2 | 18.2 | 18.2 | 1.000 | 1.000 | 0.330 | 1.349 |
| 279 | 24.2 | 12.1 | 0.0 | 1.614 | 2.348 | 1.498 | 1.916 |
| 298 | 21.2 | 18.2 | 18.2 | 1.000 | 1.000 | 0.330 | 1.349 |
| 329 | 21.2 | 9.1 | 6.1 | 1.000 | 1.000 | 2.211 | 1.182 |
| 353 | 45.5 | 12.1 | 3.0 | 1.870 | 3.104 | 1.361 | 2.388 |
| 354 | 48.5 | 30.3 | 3.0 | 1.000 | 1.592 | 2.248 | 2.315 |
| 381 | 27.3 | 18.2 | 6.1 | 1.000 | 1.620 | 1.822 | 1.692 |
| 389 | 21.2 | 9.1 | 6.1 | 1.000 | 1.000 | 2.211 | 1.182 |
| 405 | 21.2 | 9.1 | 3.0 | 1.000 | 2.366 | 1.546 | 1.562 |
| 406 | 21.2 | 9.1 | 3.0 | 1.000 | 2.366 | 1.546 | 1.562 |
| 412 | 36.4 | 18.2 | 0.0 | 2.584 | 1.332 | 1.952 | 1.641 |
| 421 | 51.5 | 24.2 | 3.0 | 2.481 | 2.253 | 2.234 | 1.431 |
| 465 | 21.2 | 18.2 | 15.2 | 1000.000 | 0.001 | 2.345 | 1.000 |
| 534 | 21.2 | 9.1 | 3.0 | 1.000 | 2.366 | 1.546 | 1.562 |
| 538 | 42.4 | 15.2 | 0.0 | 1.489 | 2.019 | 3.022 | 1.121 |
| 565 | 45.5 | 12.1 | 3.0 | 1.870 | 3.104 | 1.361 | 2.388 |
| 657 | 45.5 | 30.3 | 3.0 | 1.512 | 2.748 | 0.784 | 2.162 |
| 670 | 24.2 | 6.1 | 0.0 | 1.190 | 1.000 | 0.656 | 1.456 |
| 739 | 21.2 | 12.1 | 0.0 | 1.936 | 1.830 | 0.831 | 1.347 |
| 741 | 48.5 | 18.2 | 0.0 | 2.750 | 2.458 | 1.485 | 1.151 |
| 744 | 48.5 | 21.2 | 0.0 | 2.069 | 3.002 | 1.229 | 1.631 |
| 755 | 30.3 | 18.2 | 3.0 | 1.000 | 1.414 | 1.236 | 1.738 |
| 757 | 21.2 | 15.2 | 6.1 | 1.000 | 0.839 | 2.032 | 2.557 |
| 810 | 30.3 | 18.2 | 3.0 | 1.000 | 1.414 | 1.236 | 1.738 |
| 811 | 30.3 | 18.2 | 3.0 | 1.000 | 1.414 | 1.236 | 1.738 |
| 845 | 30.3 | 15.2 | 9.1 | 1.000 | 0.271 | 0.860 | 1.310 |
| 861 | 24.2 | 21.2 | 15.2 | 1000.000 | 1000.000 | 1.000 | 1.320 |
| 915 | 30.3 | 15.2 | 3.0 | 1.855 | 2.705 | 1.000 | 2.280 |
| 954 | 24.2 | 21.2 | 15.2 | 1000.000 | 1000.000 | 1.000 | 1.320 |
| 955 | 39.4 | 21.2 | 3.0 | 1.612 | 2.281 | 0.785 | 2.045 |
| 991 | 39.4 | 21.2 | 3.0 | 1.612 | 2.281 | 0.785 | 2.045 |
| 1035 | 24.2 | 6.1 | 0.0 | 1.190 | 1.000 | 0.656 | 1.456 |
| 1049 | 21.2 | 12.1 | 0.0 | 1.936 | 1.830 | 0.831 | 1.347 |
| 1050 | 39.4 | 21.2 | 3.0 | 1.612 | 2.281 | 0.785 | 2.045 |
| 1235 | 45.5 | 12.1 | 3.0 | 1.870 | 3.104 | 1.361 | 2.388 |
| 1292 | 21.2 | 3.0 | 0.0 | 1.558 | 2.014 | 2.250 | 1.643 |
| 1313 | 24.2 | 6.1 | 0.0 | 1.190 | 1.000 | 0.656 | 1.456 |
| 1331 | 27.3 | 9.1 | 3.0 | 1.327 | 3.749 | 1.000 | 2.045 |
| 1334 | 48.5 | 30.3 | 3.0 | 1.000 | 1.592 | 2.248 | 2.315 |
| 1418 | 30.3 | 15.2 | 3.0 | 1.855 | 2.705 | 1.000 | 2.280 |
| 1419 | 45.5 | 12.1 | 3.0 | 1.870 | 3.104 | 1.361 | 2.388 |
| 1420 | 39.4 | 18.2 | 3.0 | 1.759 | 1.566 | 1.000 | 2.302 |
| 1477 | 39.4 | 18.2 | 3.0 | 1.759 | 1.566 | 1.000 | 2.302 |
| 1554 | 33.3 | 15.2 | 0.0 | 1.829 | 1.622 | 1.882 | 1.957 |
| 1579 | 42.4 | 9.1 | 0.0 | 2.211 | 2.347 | 1.000 | 1.493 |
| 1600 | 45.5 | 12.1 | 3.0 | 1.870 | 3.104 | 1.361 | 2.388 |
| 1639 | 48.5 | 27.3 | 12.1 | 1.735 | 3.110 | 1.379 | 2.277 |
| 1657 | 42.4 | 24.2 | 0.0 | 1.000 | 1.908 | 2.267 | 1.188 |
| 1679 | 33.3 | 15.2 | 0.0 | 1.829 | 1.622 | 1.882 | 1.957 |
| 1744 | 42.4 | 15.2 | 3.0 | 2.059 | 2.753 | 1.679 | 1.587 |
| 1847 | 78.8 | 63.6 | 9.1 | 2.625 | 4.493 | 1.642 | 2.743 |
| 1877 | 45.5 | 12.1 | 3.0 | 1.870 | 3.104 | 1.361 | 2.388 |
| 1880 | 66.7 | 48.5 | 6.1 | 1.000 | 4.075 | 1.754 | 2.436 |
| 1889 | 45.5 | 12.1 | 3.0 | 1.870 | 3.104 | 1.361 | 2.388 |
| 2009 | 30.3 | 15.2 | 3.0 | 1.855 | 2.705 | 1.000 | 2.280 |
| 2023 | 30.3 | 18.2 | 0.0 | 1.285 | 2.400 | 0.767 | 1.270 |
| 2029 | 42.4 | 9.1 | 0.0 | 2.211 | 2.347 | 1.000 | 1.493 |
| 2071 | 24.2 | 6.1 | 0.0 | 1.190 | 1.000 | 0.656 | 1.456 |
| 2077 | 24.2 | 6.1 | 0.0 | 1.190 | 1.000 | 0.656 | 1.456 |
| 2103 | 27.3 | 21.2 | 0.0 | 3.505 | 0.793 | 0.809 | 1.348 |
| 2109 | 24.2 | 6.1 | 0.0 | 1.190 | 1.000 | 0.656 | 1.456 |
| 2138 | 33.3 | 21.2 | 9.1 | 1.000 | 0.296 | 3.016 | 0.794 |
| 2143 | 60.6 | 48.5 | 12.1 | 6.263 | 1.000 | 1.832 | 1.937 |
| 2183 | 63.6 | 45.5 | 12.1 | 1.945 | 2.010 | 0.547 | 3.325 |
| 2185 | 30.3 | 18.2 | 3.0 | 1.000 | 1.414 | 1.236 | 1.738 |
| 2190 | 24.2 | 6.1 | 0.0 | 1.190 | 1.000 | 0.656 | 1.456 |
| 2200 | 45.5 | 12.1 | 3.0 | 1.870 | 3.104 | 1.361 | 2.388 |
| 2206 | 60.6 | 27.3 | 3.0 | 2.256 | 2.228 | 1.673 | 1.937 |
| 2220 | 33.3 | 24.2 | 3.0 | 2.591 | 0.483 | 2.580 | 1.440 |
| 2224 | 48.5 | 36.4 | 3.0 | 1.602 | 3.209 | 1.000 | 2.942 |
| 2249 | 45.5 | 12.1 | 3.0 | 1.870 | 3.104 | 1.361 | 2.388 |
| 2255 | 24.2 | 3.0 | 0.0 | 1.985 | 2.261 | 1.000 | 0.904 |
| 2265 | 54.5 | 42.4 | 6.1 | 1.886 | 1.000 | 1.503 | 3.375 |
| 2267 | 84.8 | 57.6 | 18.2 | 2.529 | 3.042 | 2.471 | 1.669 |
| 2281 | 54.5 | 36.4 | 3.0 | 2.008 | 0.686 | 3.104 | 1.362 |
| 2294 | 30.3 | 15.2 | 3.0 | 1.855 | 2.705 | 1.000 | 2.280 |
| 2300 | 30.3 | 15.2 | 3.0 | 1.855 | 2.705 | 1.000 | 2.280 |
| 2307 | 42.4 | 9.1 | 0.0 | 2.211 | 2.347 | 1.000 | 1.493 |
| 2309 | 42.4 | 21.2 | 9.1 | 2.497 | 1.837 | 3.249 | 1.497 |
| 2313 | 57.6 | 48.5 | 9.1 | 2.603 | 2.642 | 1.000 | 1.939 |
| 2314 | 48.5 | 27.3 | 12.1 | 1.735 | 3.110 | 1.379 | 2.277 |
| 2316 | 42.4 | 9.1 | 0.0 | 2.211 | 2.347 | 1.000 | 1.493 |
| 2327 | 39.4 | 24.2 | 3.0 | 2.006 | 1.692 | 1.778 | 1.662 |
| 2348 | 72.7 | 45.5 | 0.0 | 2.961 | 3.152 | 2.712 | 1.346 |

TABLE 11

| SEQ ID | | | | | | | | |
|---|---|---|---|---|---|---|---|---|
| NO | P128 | P130 | P133 | P141 | P156 | P228 | P264 | P266 |
| 18 | 0.713 | 1.800 | 1.955 | 0.663 | 0.466 | 1.457 | 2.262 | 1.236 |
| 22 | 1.594 | 6.800 | 1.340 | 1.131 | 1.000 | 2.647 | 1.628 | 1.190 |
| 127 | 3.761 | 1.000 | 1.000 | 1.587 | 2.127 | 1.000 | 1.000 | 1.000 |
| 139 | 1000.000 | 1.000 | 1000.000 | 0.482 | 2.846 | 0.767 | 1.631 | 1.000 |
| 148 | 3.761 | 1.000 | 1.000 | 1.587 | 2.127 | 1.000 | 1.000 | 1.000 |
| 155 | 2.062 | 1.781 | 2.302 | 1.000 | 1.000 | 1.306 | 2.099 | 1.357 |
| 246 | 1.779 | 1.337 | 2.865 | 1.515 | 1.617 | 1.301 | 2.098 | 1.733 |
| 272 | 2.044 | 2.219 | 4.257 | 0.744 | 1.000 | 1.127 | 1.588 | 1.634 |
| 273 | 1000.000 | 1000.000 | 1.000 | 1.000 | 0.566 | 1.554 | 1.000 | 1.000 |
| 279 | 1.202 | 1.852 | 2.370 | 1.000 | 1.000 | 1.114 | 1.399 | 1.239 |
| 298 | 1000.000 | 1000.000 | 1.000 | 1.000 | 0.566 | 1.554 | 1.000 | 1.000 |
| 329 | 3.234 | 0.001 | 1.000 | 8.480 | 2.077 | 1.000 | 0.001 | 1.445 |
| 353 | 2.062 | 1.781 | 2.302 | 1.000 | 1.000 | 1.306 | 2.099 | 1.357 |
| 354 | 1.664 | 1.987 | 2.307 | 2.728 | 1.000 | 1.239 | 1.469 | 2.059 |
| 381 | 3.761 | 1.000 | 1.000 | 1.587 | 2.127 | 1.000 | 1.000 | 1.000 |
| 389 | 3.234 | 0.001 | 1.000 | 8.480 | 2.077 | 1.000 | 0.001 | 1.445 |
| 405 | 1.531 | 1.553 | 1.854 | 2.044 | 1.363 | 1.786 | 1.877 | 1.644 |
| 406 | 1.531 | 1.553 | 1.854 | 2.044 | 1.363 | 1.786 | 1.877 | 1.644 |
| 412 | 1.831 | 1.503 | 2.326 | 1.130 | 1.773 | 1.379 | 2.318 | 2.019 |
| 421 | 2.209 | 1.889 | 3.114 | 1.776 | 1.788 | 1.879 | 2.666 | 2.257 |
| 465 | 1000.000 | 1.000 | 1000.000 | 0.482 | 2.846 | 0.767 | 1.631 | 1.000 |
| 534 | 1.531 | 1.553 | 1.854 | 2.044 | 1.363 | 1.786 | 1.877 | 1.644 |
| 538 | 1.559 | 1.000 | 1.740 | 3.133 | 2.186 | 1.869 | 2.023 | 2.483 |
| 565 | 2.062 | 1.781 | 2.302 | 1.000 | 1.000 | 1.306 | 2.099 | 1.357 |
| 657 | 1.524 | 1.770 | 2.846 | 1.185 | 1.000 | 1.460 | 1.831 | 2.261 |
| 670 | 1.182 | 1.636 | 1.418 | 1.298 | 1.000 | 1.000 | 1.127 | 0.774 |
| 739 | 0.845 | 1.286 | 1.872 | 1.000 | 1.000 | 1.295 | 1.722 | 1.785 |
| 741 | 1.819 | 1.801 | 3.227 | 1.457 | 2.960 | 1.388 | 2.086 | 2.410 |
| 744 | 2.515 | 1.605 | 2.399 | 1.803 | 2.524 | 1.551 | 2.284 | 1.574 |
| 755 | 1.000 | 0.754 | 2.234 | 3.723 | 1.000 | 1.285 | 1.771 | 2.246 |
| 757 | 0.745 | 1.332 | 1000.000 | 1.000 | 1.000 | 1.781 | 1.515 | 1.747 |
| 810 | 1.000 | 0.754 | 2.234 | 3.723 | 1.000 | 1.285 | 1.771 | 2.246 |
| 811 | 1.000 | 0.754 | 2.234 | 3.723 | 1.000 | 1.285 | 1.771 | 2.246 |
| 845 | 2.331 | 1.641 | 1000.000 | 1.252 | 1.000 | 0.595 | 1.950 | 0.616 |
| 861 | 2.888 | 1.000 | 0.001 | 1.000 | 1.694 | 0.001 | 1000.000 | 1.423 |
| 915 | 0.713 | 1.800 | 1.955 | 0.663 | 0.466 | 1.457 | 2.262 | 1.236 |
| 954 | 2.888 | 1.000 | 0.001 | 1.000 | 1.694 | 0.001 | 1000.000 | 1.423 |
| 955 | 1.415 | 2.042 | 2.733 | 0.898 | 1.431 | 1.000 | 1.459 | 2.009 |
| 991 | 1.415 | 2.042 | 2.733 | 0.898 | 1.431 | 1.000 | 1.459 | 2.009 |
| 1035 | 1.182 | 1.636 | 1.418 | 1.298 | 1.000 | 1.000 | 1.127 | 0.774 |
| 1049 | 0.845 | 1.286 | 1.872 | 1.000 | 1.000 | 1.295 | 1.722 | 1.785 |
| 1050 | 1.415 | 2.042 | 2.733 | 0.898 | 1.431 | 1.000 | 1.459 | 2.009 |
| 1235 | 2.062 | 1.781 | 2.302 | 1.000 | 1.000 | 1.306 | 2.099 | 1.357 |
| 1292 | 1.804 | 1.641 | 1.876 | 1.335 | 0.766 | 1.245 | 1.500 | 1.000 |
| 1313 | 1.182 | 1.636 | 1.418 | 1.298 | 1.000 | 1.000 | 1.127 | 0.774 |
| 1331 | 1.427 | 1.669 | 1.837 | 1.265 | 1.000 | 1.667 | 1.000 | 1.374 |
| 1334 | 1.664 | 1.987 | 2.307 | 2.728 | 1.000 | 1.239 | 1.469 | 2.059 |
| 1418 | 0.713 | 1.800 | 1.955 | 0.663 | 0.466 | 1.457 | 2.262 | 1.236 |
| 1419 | 2.062 | 1.781 | 2.302 | 1.000 | 1.000 | 1.306 | 2.099 | 1.357 |
| 1420 | 1.518 | 1.997 | 2.298 | 2.273 | 1.000 | 1.234 | 1.186 | 1.730 |
| 1477 | 1.518 | 1.997 | 2.298 | 2.273 | 1.000 | 1.234 | 1.186 | 1.730 |
| 1554 | 2.959 | 1.821 | 2.234 | 1.181 | 1.827 | 1.000 | 2.042 | 1.970 |
| 1579 | 1.779 | 1.337 | 2.865 | 1.515 | 1.617 | 1.301 | 2.098 | 1.733 |
| 1600 | 2.062 | 1.781 | 2.302 | 1.000 | 1.000 | 1.306 | 2.099 | 1.357 |
| 1639 | 2.044 | 2.219 | 4.257 | 0.744 | 1.000 | 1.127 | 1.588 | 1.634 |
| 1657 | 2.160 | 1.416 | 1.000 | 3.531 | 2.974 | 1.798 | 1.899 | 2.065 |
| 1679 | 2.959 | 1.821 | 2.234 | 1.181 | 1.827 | 1.000 | 2.042 | 1.970 |
| 1744 | 1.479 | 1.669 | 2.442 | 1.352 | 1.367 | 1.605 | 2.145 | 2.098 |
| 1847 | 1.839 | 2.548 | 2.954 | 2.234 | 1.816 | 1.352 | 3.390 | 2.541 |
| 1877 | 2.062 | 1.781 | 2.302 | 1.000 | 1.000 | 1.306 | 2.099 | 1.357 |
| 1880 | 2.762 | 2.081 | 4.111 | 2.306 | 2.391 | 1.675 | 2.572 | 3.031 |
| 1889 | 2.062 | 1.781 | 2.302 | 1.000 | 1.000 | 1.306 | 2.099 | 1.357 |
| 2009 | 0.713 | 1.800 | 1.955 | 0.663 | 0.466 | 1.457 | 2.262 | 1.236 |
| 2023 | 1.871 | 1.869 | 2.588 | 1.834 | 1.718 | 1.197 | 1.965 | 2.023 |
| 2029 | 1.779 | 1.337 | 2.865 | 1.515 | 1.617 | 1.301 | 2.098 | 1.733 |
| 2071 | 1.182 | 1.636 | 1.418 | 1.298 | 1.000 | 1.000 | 1.127 | 0.774 |
| 2077 | 1.182 | 1.636 | 1.418 | 1.298 | 1.000 | 1.000 | 1.127 | 0.774 |
| 2103 | 2.297 | 0.855 | 1.659 | 1.607 | 0.252 | 1.602 | 2.866 | 1.292 |
| 2109 | 1.182 | 1.636 | 1.418 | 1.298 | 1.000 | 1.000 | 1.127 | 0.774 |
| 2138 | 2.074 | 1.438 | 1.552 | 2.403 | 0.647 | 0.605 | 0.469 | 0.528 |
| 2143 | 2.828 | 2.795 | 2.732 | 2.548 | 0.073 | 1.201 | 1.722 | 1.181 |
| 2183 | 1.714 | 3.061 | 4.635 | 1.688 | 1.230 | 1.241 | 1.237 | 1.852 |
| 2185 | 1.000 | 0.754 | 2.234 | 3.723 | 1.000 | 1.285 | 1.771 | 2.246 |
| 2190 | 1.182 | 1.636 | 1.418 | 1.298 | 1.000 | 1.000 | 1.127 | 0.774 |
| 2200 | 2.062 | 1.781 | 2.302 | 1.000 | 1.000 | 1.306 | 2.099 | 1.357 |
| 2206 | 2.229 | 2.131 | 2.194 | 2.235 | 2.121 | 1.388 | 3.468 | 2.115 |
| 2220 | 2.650 | 0.815 | 1.629 | 1.586 | 0.155 | 1.408 | 2.830 | 1.636 |
| 2224 | 1.385 | 2.044 | 2.510 | 0.628 | 1.763 | 1.000 | 1.000 | 1.687 |
| 2249 | 2.062 | 1.781 | 2.302 | 1.000 | 1.000 | 1.306 | 2.099 | 1.357 |
| 2255 | 1.454 | 1.000 | 1.567 | 2.350 | 1.729 | 2.071 | 1.439 | 1.540 |
| 2265 | 2.843 | 2.931 | 1.690 | 1.678 | 0.724 | 2.656 | 2.035 | 3.526 |
| 2267 | 2.490 | 1.937 | 3.729 | 2.105 | 2.224 | 2.547 | 2.605 | 4.402 |
| 2281 | 3.412 | 2.374 | 1.404 | 4.761 | 3.241 | 2.253 | 1.384 | 1.912 |
| 2294 | 0.713 | 1.800 | 1.955 | 0.663 | 0.466 | 1.457 | 2.262 | 1.236 |
| 2300 | 0.713 | 1.800 | 1.955 | 0.663 | 0.466 | 1.457 | 2.262 | 1.236 |
| 2307 | 1.779 | 1.337 | 2.865 | 1.515 | 1.617 | 1.301 | 2.098 | 1.733 |
| 2309 | 1.496 | 1.483 | 2.427 | 1.764 | 1.000 | 1.231 | 1.413 | 1.000 |
| 2313 | 1.452 | 1.915 | 2.252 | 1.342 | 2.516 | 1.278 | 2.179 | 4.223 |
| 2314 | 2.044 | 2.219 | 4.257 | 0.744 | 1.000 | 1.127 | 1.588 | 1.634 |
| 2316 | 1.779 | 1.337 | 2.865 | 1.515 | 1.617 | 1.301 | 2.098 | 1.733 |
| 2327 | 1.778 | 1.200 | 2.169 | 1.462 | 1.570 | 1.784 | 1.937 | 2.633 |
| 2348 | 2.064 | 1.288 | 2.075 | 2.527 | 2.239 | 1.745 | 3.772 | 3.393 |
| 2384 | 2.340 | 0.001 | 0.001 | 2.927 | 4.830 | 1.708 | 1.651 | 1.586 |

TABLE 12

| SEQ ID | | | | | | | | |
|---|---|---|---|---|---|---|---|---|
| NO | P268 | P278 | P295 | P339 | P341 | P356 | P360 | P392 |
| 18 | 1.000 | 2.819 | 1.000 | 1.589 | 1.238 | 1.784 | 0.748 | 2.486 |
| 22 | 1.194 | 1.000 | 1.000 | 1.474 | 3.006 | 2.766 | 1.622 | 10.061 |
| 127 | 2.953 | 2.030 | 8.118 | 1.000 | 2.854 | 1.000 | 1000.000 | 0.001 |
| 139 | 1000.000 | 1.332 | 1.000 | 0.344 | 1.537 | 1.000 | 0.001 | 0.464 |
| 148 | 2.953 | 2.030 | 8.118 | 1.000 | 2.854 | 1.000 | 1000.000 | 0.001 |
| 155 | 1.187 | 1.447 | 1.000 | 1.484 | 3.621 | 3.844 | 1.995 | 1.313 |
| 246 | 1.422 | 2.018 | 2.385 | 1.218 | 2.039 | 3.486 | 1.636 | 1.623 |
| 272 | 1.268 | 1.563 | 1.870 | 2.056 | 6.240 | 6.491 | 2.230 | 1.427 |
| 273 | 1.000 | 1000.000 | 1.000 | 1.196 | 2.209 | 1000.000 | 0.001 | 1.000 |
| 279 | 1.000 | 1.000 | 1.000 | 1.737 | 2.382 | 3.061 | 2.679 | 1.361 |
| 298 | 1.000 | 1000.000 | 1.000 | 1.196 | 2.209 | 1000.000 | 0.001 | 1.000 |
| 329 | 2.467 | 2.166 | 21.707 | 0.615 | 1.616 | 1.000 | 1.000 | 1.000 |
| 353 | 1.187 | 1.447 | 1.000 | 1.484 | 3.621 | 3.844 | 1.995 | 1.313 |
| 354 | 2.359 | 1.552 | 2.918 | 1.647 | 4.706 | 3.623 | 1.979 | 1.677 |
| 381 | 2.953 | 2.030 | 8.118 | 1.000 | 2.854 | 1.000 | 1000.000 | 0.001 |
| 389 | 2.467 | 2.166 | 21.707 | 0.615 | 1.616 | 1.000 | 1.000 | 1.000 |
| 405 | 1.221 | 1.796 | 1.995 | 1.780 | 1.726 | 2.970 | 1.792 | 1.581 |
| 406 | 1.221 | 1.796 | 1.995 | 1.780 | 1.726 | 2.970 | 1.792 | 1.581 |
| 412 | 2.677 | 2.809 | 2.969 | 1.373 | 2.087 | 3.804 | 1.612 | 1.163 |
| 421 | 2.468 | 5.262 | 4.008 | 1.487 | 4.366 | 2.078 | 1.781 | 1.332 |
| 465 | 1000.000 | 1.332 | 1.000 | 0.344 | 1.537 | 1.000 | 0.001 | 0.464 |
| 534 | 1.221 | 1.796 | 1.995 | 1.780 | 1.726 | 2.970 | 1.792 | 1.581 |
| 538 | 2.565 | 1.856 | 1.000 | 1.000 | 2.449 | 1.000 | 2.097 | 2.647 |
| 565 | 1.187 | 1.447 | 1.000 | 1.484 | 3.621 | 3.844 | 1.995 | 1.313 |
| 657 | 1.369 | 1.000 | 1.000 | 1.679 | 3.084 | 2.855 | 2.104 | 0.927 |
| 670 | 1.677 | 2.420 | 2.263 | 1.314 | 1.473 | 2.523 | 1.776 | 2.244 |
| 739 | 1.412 | 1.431 | 3.103 | 1.000 | 2.847 | 2.621 | 1.000 | 1.117 |
| 741 | 2.240 | 2.040 | 1.000 | 1.000 | 2.450 | 3.440 | 2.045 | 1.998 |
| 744 | 1.837 | 2.201 | 2.518 | 1.604 | 2.248 | 2.989 | 1.570 | 1.409 |
| 755 | 1.000 | 1.320 | 0.556 | 1.385 | 1.321 | 1.000 | 1.000 | 6.185 |
| 757 | 0.713 | 1000.000 | 0.632 | 2.389 | 0.202 | 1.000 | 1.000 | 0.356 |
| 810 | 1.000 | 1.320 | 0.556 | 1.385 | 1.321 | 1.000 | 1.000 | 6.185 |
| 811 | 1.000 | 1.320 | 0.556 | 1.385 | 1.321 | 1.000 | 1.000 | 6.185 |
| 845 | 2.151 | 2.384 | 2.417 | 0.573 | 1.451 | 2.652 | 1.000 | 0.734 |
| 861 | 1.000 | 1.509 | 9.879 | 1000.000 | 2.327 | 0.001 | 1.236 | 0.870 |
| 915 | 1.000 | 2.819 | 1.000 | 1.589 | 1.238 | 1.784 | 0.748 | 2.486 |
| 954 | 1.000 | 1.509 | 9.879 | 1000.000 | 2.327 | 0.001 | 1.236 | 0.870 |
| 955 | 1.657 | 1.732 | 3.510 | 1.652 | 4.946 | 4.071 | 2.194 | 1.932 |
| 991 | 1.657 | 1.732 | 3.510 | 1.652 | 4.946 | 4.071 | 2.194 | 1.932 |
| 1035 | 1.677 | 2.420 | 2.263 | 1.314 | 1.473 | 2.523 | 1.776 | 2.244 |
| 1049 | 1.412 | 1.431 | 3.103 | 1.000 | 2.847 | 2.621 | 1.000 | 1.117 |
| 1050 | 1.657 | 1.732 | 3.510 | 1.652 | 4.946 | 4.071 | 2.194 | 1.932 |
| 1235 | 1.187 | 1.447 | 1.000 | 1.484 | 3.621 | 3.844 | 1.995 | 1.313 |
| 1292 | 0.718 | 1.000 | 1.000 | 1.675 | 2.301 | 1.361 | 2.161 | 1.825 |
| 1313 | 1.677 | 2.420 | 2.263 | 1.314 | 1.473 | 2.523 | 1.776 | 2.244 |
| 1331 | 0.789 | 1.609 | 1.000 | 0.797 | 1.000 | 2.075 | 2.491 | 2.505 |
| 1334 | 2.359 | 1.552 | 2.918 | 1.647 | 4.706 | 3.623 | 1.979 | 1.677 |
| 1418 | 1.000 | 2.819 | 1.000 | 1.589 | 1.238 | 1.784 | 0.748 | 2.486 |
| 1419 | 1.187 | 1.447 | 1.000 | 1.484 | 3.621 | 3.844 | 1.995 | 1.313 |
| 1420 | 1.864 | 1.428 | 2.631 | 1.854 | 3.430 | 3.182 | 1.892 | 1.581 |
| 1477 | 1.864 | 1.428 | 2.631 | 1.854 | 3.430 | 3.182 | 1.892 | 1.581 |
| 1554 | 2.495 | 2.090 | 3.320 | 1.000 | 3.907 | 2.976 | 1.875 | 1.000 |
| 1579 | 1.422 | 2.018 | 2.385 | 1.218 | 2.039 | 3.486 | 1.636 | 1.623 |
| 1600 | 1.187 | 1.447 | 1.000 | 1.484 | 3.621 | 3.844 | 1.995 | 1.313 |
| 1639 | 1.268 | 1.563 | 1.870 | 2.056 | 6.240 | 6.491 | 2.230 | 1.427 |
| 1657 | 2.183 | 2.285 | 3.554 | 1.247 | 2.093 | 1.840 | 1.855 | 1.504 |
| 1679 | 2.495 | 2.090 | 3.320 | 1.000 | 3.907 | 2.976 | 1.875 | 1.000 |
| 1744 | 2.006 | 1.696 | 2.261 | 1.611 | 2.154 | 3.791 | 1.816 | 1.356 |
| 1847 | 1.535 | 2.851 | 4.154 | 2.055 | 6.047 | 4.103 | 3.367 | 2.029 |
| 1877 | 1.187 | 1.447 | 1.000 | 1.484 | 3.621 | 3.844 | 1.995 | 1.313 |
| 1880 | 2.274 | 1.266 | 4.526 | 2.591 | 5.409 | 3.138 | 2.675 | 1.391 |
| 1889 | 1.187 | 1.447 | 1.000 | 1.484 | 3.621 | 3.844 | 1.995 | 1.313 |
| 2009 | 1.000 | 2.819 | 1.000 | 1.589 | 1.238 | 1.784 | 0.748 | 2.486 |
| 2023 | 1.971 | 1.699 | 2.355 | 1.453 | 3.122 | 2.528 | 1.949 | 1.326 |
| 2029 | 1.422 | 2.018 | 2.385 | 1.218 | 2.039 | 3.486 | 1.636 | 1.623 |
| 2071 | 1.677 | 2.420 | 2.263 | 1.314 | 1.473 | 2.523 | 1.776 | 2.244 |
| 2077 | 1.677 | 2.420 | 2.263 | 1.314 | 1.473 | 2.523 | 1.776 | 2.244 |
| 2103 | 2.516 | 0.852 | 1.775 | 0.818 | 4.294 | 2.281 | 1.119 | 0.890 |
| 2109 | 1.677 | 2.420 | 2.263 | 1.314 | 1.473 | 2.523 | 1.776 | 2.244 |
| 2138 | 1.794 | 1.486 | 5.006 | 0.398 | 4.768 | 0.001 | 2.344 | 2.434 |
| 2143 | 2.079 | 1.664 | 1.000 | 1.871 | 2.812 | 2.693 | 5.094 | 1.947 |
| 2183 | 2.325 | 2.043 | 2.530 | 2.411 | 5.749 | 5.509 | 3.490 | 2.008 |
| 2185 | 1.000 | 1.320 | 0.556 | 1.385 | 1.321 | 1.000 | 1.000 | 6.185 |
| 2190 | 1.677 | 2.420 | 2.263 | 1.314 | 1.473 | 2.523 | 1.776 | 2.244 |
| 2200 | 1.187 | 1.447 | 1.000 | 1.484 | 3.621 | 3.844 | 1.995 | 1.313 |
| 2206 | 1.977 | 1.676 | 1.774 | 1.542 | 2.538 | 1.867 | 2.312 | 1.000 |
| 2220 | 2.942 | 0.729 | 1.772 | 0.861 | 5.794 | 2.349 | 1.363 | 0.808 |
| 2224 | 1.457 | 1.690 | 2.551 | 1.860 | 4.114 | 3.548 | 3.125 | 0.792 |
| 2249 | 1.18 | 1.447 | 1.000 | 1.484 | 3.621 | 3.844 | 1.995 | 1.313 |
| 2255 | 1.586 | 1.943 | 1.000 | 0.699 | 1.593 | 2.039 | 1.798 | 0.774 |
| 2265 | 2.157 | 1.922 | 3.895 | 4.143 | 2.655 | 1.914 | 2.159 | 3.312 |
| 2267 | 3.442 | 3.933 | 5.994 | 1.448 | 8.695 | 7.488 | 2.687 | 2.449 |
| 2281 | 2.467 | 1.000 | 7.584 | 1.417 | 3.693 | 1.947 | 1.539 | 4.429 |
| 2294 | 1.000 | 2.819 | 1.000 | 1.589 | 1.238 | 1.784 | 0.748 | 2.486 |
| 2300 | 1.000 | 2.819 | 1.000 | 1.589 | 1.238 | 1.784 | 0.748 | 2.486 |
| 2307 | 1.422 | 2.018 | 2.385 | 1.218 | 2.039 | 3.486 | 1.636 | 1.623 |
| 2309 | 2.485 | 2.369 | 1.000 | 1.820 | 3.354 | 5.046 | 1.820 | 0.703 |
| 2313 | 3.203 | 1.593 | 4.012 | 1.593 | 6.374 | 6.940 | 3.158 | 0.947 |
| 2314 | 1.268 | 1.563 | 1.870 | 2.056 | 6.240 | 6.491 | 2.230 | 1.427 |
| 2316 | 1.422 | 2.018 | 2.385 | 1.218 | 2.039 | 3.486 | 1.636 | 1.623 |
| 2327 | 2.439 | 1.482 | 2.156 | 1.390 | 3.500 | 3.654 | 1.655 | 0.771 |
| 2348 | 2.448 | 2.617 | 4.003 | 1.289 | 2.940 | 3.894 | 2.277 | 1.202 |
| 2384 | 2.328 | 1.359 | 9.253 | 0.383 | 1.835 | 0.001 | 1.000 | 0.714 |

TABLE 13

| SEQ ID | | | | | | | | |
|---|---|---|---|---|---|---|---|---|
| NO | P393 | P413 | P505 | P517 | P534 | P546 | P577 | P695 |
| 18 | 1.058 | 2.471 | 1.583 | 1.726 | 0.506 | 1.431 | 2.632 | 5.930 |
| 22 | 14.260 | 2.516 | 1.498 | 3.747 | 1.300 | 5.779 | 11.202 | 0.001 |
| 127 | 1.000 | 0.001 | 1.000 | 1.000 | 0.001 | 1.000 | 3.303 | 1.000 |
| 139 | 1.000 | 1.000 | 0.458 | 1.249 | 0.001 | 1000.000 | 0.702 | 1.000 |
| 148 | 1.000 | 0.001 | 1.000 | 1.000 | 0.001 | 1.000 | 3.303 | 1.000 |
| 155 | 1.137 | 2.268 | 2.414 | 1.382 | 2.107 | 2.210 | 2.384 | 5.256 |
| 246 | 0.741 | 2.181 | 2.494 | 1.504 | 1.511 | 1.831 | 2.064 | 4.421 |
| 272 | 1.348 | 2.222 | 2.506 | 1.355 | 1.670 | 2.535 | 1.556 | 8.411 |
| 273 | 1.000 | 1.000 | 1000.000 | 1.477 | 1.645 | 1.000 | 1.389 | 1.000 |
| 279 | 0.914 | 1.603 | 1.936 | 1.485 | 2.430 | 1.999 | 1.647 | 4.375 |
| 298 | 1.000 | 1.000 | 1000.000 | 1.477 | 1.645 | 1.000 | 1.389 | 1.000 |
| 329 | 1.000 | 1.000 | 1.436 | 0.517 | 1.000 | 1.469 | 1.000 | 1.000 |
| 353 | 1.137 | 2.268 | 2.414 | 1.382 | 2.107 | 2.210 | 2.384 | 5.256 |
| 354 | 1.224 | 3.432 | 2.806 | 1.328 | 2.470 | 2.592 | 1.929 | 6.973 |
| 381 | 1.000 | 0.001 | 1.000 | 1.000 | 0.001 | 1.000 | 3.303 | 1.000 |
| 389 | 1.000 | 1.000 | 1.436 | 0.517 | 1.000 | 1.469 | 1.000 | 1.000 |
| 405 | 1.241 | 1.841 | 1.470 | 1.000 | 1.672 | 2.218 | 1.649 | 7.555 |
| 406 | 1.241 | 1.841 | 1.470 | 1.000 | 1.672 | 2.218 | 1.649 | 7.555 |
| 412 | 1.258 | 2.153 | 1.849 | 1.445 | 1.000 | 1.531 | 1.637 | 3.302 |
| 421 | 1.000 | 1.327 | 2.871 | 1.116 | 1.903 | 2.200 | 2.644 | 0.001 |
| 465 | 1.000 | 1.000 | 0.458 | 1.249 | 0.001 | 1000.000 | 0.702 | 1.000 |
| 534 | 1.241 | 1.841 | 1.470 | 1.000 | 1.672 | 2.218 | 1.649 | 7.555 |
| 538 | 1.560 | 1.982 | 2.159 | 1.278 | 1.425 | 1.204 | 3.046 | 2.068 |
| 565 | 1.137 | 2.268 | 2.414 | 1.382 | 2.107 | 2.210 | 2.384 | 5.256 |
| 657 | 0.763 | 1.602 | 2.797 | 1.265 | 2.765 | 2.236 | 2.548 | 5.071 |
| 670 | 1.710 | 2.337 | 1.898 | 0.892 | 1.347 | 1.908 | 1.136 | 3.404 |
| 739 | 2.102 | 1.689 | 4.429 | 0.830 | 1.000 | 1.000 | 2.108 | 2.208 |
| 741 | 1.935 | 1.911 | 2.812 | 1.000 | 1.854 | 1.793 | 2.441 | 0.001 |
| 744 | 1.320 | 1.404 | 1.553 | 1.000 | 1.957 | 1.816 | 2.156 | 3.745 |
| 755 | 1.219 | 2.547 | 1.288 | 2.539 | 3.936 | 3.625 | 2.363 | 1.955 |
| 757 | 0.851 | 0.750 | 0.815 | 0.258 | 0.712 | 1.229 | 0.190 | 1.000 |
| 810 | 1.219 | 2.547 | 1.288 | 2.539 | 3.936 | 3.625 | 2.363 | 1.955 |
| 811 | 1.219 | 2.547 | 1.288 | 2.539 | 3.936 | 3.625 | 2.363 | 1.955 |
| 845 | 2.765 | 1.000 | 2.202 | 0.472 | 0.490 | 1.417 | 0.725 | 0.001 |
| 861 | 1.000 | 1.000 | 1.000 | 1.000 | 1.000 | 1.530 | 0.769 | 1.000 |
| 915 | 1.058 | 2.471 | 1.583 | 1.726 | 0.506 | 1.431 | 2.632 | 5.930 |
| 954 | 1.000 | 1.000 | 1.000 | 1.000 | 1.000 | 1.530 | 0.769 | 1.000 |
| 955 | 1.322 | 2.608 | 1.910 | 1.199 | 1.635 | 1.893 | 1.473 | 5.842 |
| 991 | 1.322 | 2.608 | 1.910 | 1.199 | 1.635 | 1.893 | 1.473 | 5.842 |
| 1035 | 1.710 | 2.337 | 1.898 | 0.892 | 1.347 | 1.908 | 1.136 | 3.404 |
| 1049 | 2.102 | 1.689 | 4.429 | 0.830 | 1.000 | 1.000 | 2.108 | 2.208 |
| 1050 | 1.322 | 2.608 | 1.910 | 1.199 | 1.635 | 1.893 | 1.473 | 5.842 |
| 1235 | 1.137 | 2.268 | 2.414 | 1.382 | 2.107 | 2.210 | 2.384 | 5.256 |
| 1292 | 1.000 | 1.518 | 1.980 | 1.518 | 2.526 | 1.588 | 1.865 | 2.251 |
| 1313 | 1.710 | 2.337 | 1.898 | 0.892 | 1.347 | 1.908 | 1.136 | 3.404 |
| 1331 | 0.743 | 2.126 | 1.613 | 1.177 | 2.128 | 1.000 | 1.951 | 6.931 |
| 1334 | 1.224 | 3.432 | 2.806 | 1.328 | 2.470 | 2.592 | 1.929 | 6.973 |
| 1418 | 1.058 | 2.471 | 1.583 | 1.726 | 0.506 | 1.431 | 2.632 | 5.930 |
| 1419 | 1.137 | 2.268 | 2.414 | 1.382 | 2.107 | 2.210 | 2.384 | 5.256 |
| 1420 | 1.205 | 3.301 | 2.749 | 1.256 | 2.474 | 2.345 | 1.826 | 8.108 |
| 1477 | 1.205 | 3.301 | 2.749 | 1.256 | 2.474 | 2.345 | 1.826 | 8.108 |
| 1554 | 1.000 | 1.793 | 2.719 | 1.679 | 1.000 | 1.549 | 2.076 | 0.001 |
| 1579 | 0.741 | 2.181 | 2.494 | 1.504 | 1.511 | 1.831 | 2.064 | 4.421 |
| 1600 | 1.137 | 2.268 | 2.414 | 1.382 | 2.107 | 2.210 | 2.384 | 5.256 |
| 1639 | 1.348 | 2.222 | 2.506 | 1.355 | 1.670 | 2.535 | 1.556 | 8.411 |
| 1657 | 2.809 | 1.534 | 1.366 | 1.197 | 2.545 | 1.964 | 1.506 | 0.001 |
| 1679 | 1.000 | 1.793 | 2.719 | 1.679 | 1.000 | 1.549 | 2.076 | 0.001 |
| 1744 | 1.249 | 2.009 | 1.832 | 1.488 | 1.379 | 1.975 | 2.128 | 13.930 |
| 1847 | 1.781 | 2.929 | 2.183 | 2.759 | 3.853 | 3.092 | 2.051 | 7.549 |
| 1877 | 1.137 | 2.268 | 2.414 | 1.382 | 2.107 | 2.210 | 2.384 | 5.256 |
| 1880 | 1.000 | 3.187 | 2.564 | 0.756 | 1.226 | 3.841 | 3.201 | 16.724 |
| 1889 | 1.137 | 2.268 | 2.414 | 1.382 | 2.107 | 2.210 | 2.384 | 5.256 |
| 2009 | 1.058 | 2.471 | 1.583 | 1.726 | 0.506 | 1.431 | 2.632 | 5.930 |
| 2023 | 1.952 | 1.472 | 1.917 | 1.516 | 2.305 | 2.677 | 2.620 | 2.660 |
| 2029 | 0.741 | 2.181 | 2.494 | 1.504 | 1.511 | 1.831 | 2.064 | 4.421 |
| 2071 | 1.710 | 2.337 | 1.898 | 0.892 | 1.347 | 1.908 | 1.136 | 3.404 |
| 2077 | 1.710 | 2.337 | 1.898 | 0.892 | 1.347 | 1.908 | 1.136 | 3.404 |
| 2103 | 0.537 | 1.790 | 0.727 | 0.750 | 0.329 | 1.100 | 1.239 | 0.001 |
| 2109 | 1.710 | 2.337 | 1.898 | 0.892 | 1.347 | 1.908 | 1.136 | 3.404 |
| 2138 | 0.852 | 1.789 | 3.765 | 0.686 | 3.176 | 1.591 | 1.852 | 0.001 |
| 2143 | 2.044 | 17.760 | 4.034 | 1.988 | 0.026 | 3.908 | 2.394 | 42.662 |
| 2183 | 1.088 | 5.833 | 3.519 | 1.572 | 2.641 | 4.011 | 1.695 | 7.783 |
| 2185 | 1.219 | 2.547 | 1.288 | 2.539 | 3.936 | 3.625 | 2.363 | 1.955 |
| 2190 | 1.710 | 2.337 | 1.898 | 0.892 | 1.347 | 1.908 | 1.136 | 3.404 |
| 2200 | 1.137 | 2.268 | 2.414 | 1.382 | 2.107 | 2.210 | 2.384 | 5.256 |
| 2206 | 1.000 | 3.033 | 1.912 | 1.699 | 2.147 | 2.780 | 2.155 | 2.518 |
| 2220 | 0.337 | 2.339 | 0.768 | 0.563 | 0.359 | 1.242 | 1.492 | 1.000 |
| 2224 | 1.000 | 2.266 | 2.040 | 1.000 | 2.747 | 2.620 | 1.718 | 14.145 |
| 2249 | 1.137 | 2.268 | 2.414 | 1.382 | 2.107 | 2.210 | 2.384 | 5.256 |
| 2255 | 1.243 | 1.766 | 1.547 | 0.843 | 1.000 | 1.498 | 2.122 | 4.421 |
| 2265 | 5.268 | 1.518 | 2.253 | 3.678 | 0.766 | 1.565 | 1.000 | 1.853 |
| 2267 | 0.815 | 2.497 | 3.234 | 2.275 | 2.344 | 3.596 | 5.023 | 12.124 |
| 2281 | 1.128 | 0.885 | 1.237 | 1.434 | 3.327 | 3.206 | 1.355 | 0.001 |
| 2294 | 1.058 | 2.471 | 1.583 | 1.726 | 0.506 | 1.431 | 2.632 | 5.930 |
| 2300 | 1.058 | 2.471 | 1.583 | 1.726 | 0.506 | 1.431 | 2.632 | 5.930 |
| 2307 | 0.741 | 2.181 | 2.494 | 1.504 | 1.511 | 1.831 | 2.064 | 4.421 |
| 2309 | 1.240 | 2.239 | 2.841 | 1.000 | 2.270 | 2.614 | 0.583 | 5.244 |
| 2313 | 0.633 | 2.821 | 2.976 | 1.253 | 1.675 | 3.657 | 2.284 | 8.587 |
| 2314 | 1.348 | 2.222 | 2.506 | 1.355 | 1.670 | 2.535 | 1.556 | 8.411 |
| 2316 | 0.741 | 2.181 | 2.494 | 1.504 | 1.511 | 1.831 | 2.064 | 4.421 |
| 2327 | 1.000 | 1.801 | 1.978 | 1.000 | 3.188 | 1.607 | 2.276 | 13.068 |
| 2348 | 0.790 | 3.524 | 3.377 | 2.062 | 2.123 | 1.959 | 1.626 | 1.000 |
| 2384 | 0.001 | 1.346 | 1.831 | 1.000 | 1.646 | 1.944 | 1.549 | 1.000 |

TABLE 14

| SEQ ID | | | | | |
|---|---|---|---|---|---|
| NO | P784 | P786 | P791 | P888 | P889 |
| 18 | 1.000 | 1.000 | 4.202 | 1.464 | 2.147 |
| 22 | 1.000 | 1.276 | 14.034 | 4.139 | 3.640 |
| 127 | 1.708 | 2.247 | 1.000 | 0.441 | 0.001 |
| 139 | 1.391 | 1.857 | 1.000 | 0.402 | 1.000 |
| 148 | 1.708 | 2.247 | 1.000 | 0.441 | 0.001 |
| 155 | 1.328 | 1.421 | 2.456 | 1.910 | 2.069 |
| 246 | 1.243 | 1.679 | 2.228 | 2.333 | 1.774 |
| 272 | 0.819 | 1.632 | 2.808 | 5.465 | 2.307 |
| 273 | 1000.000 | 0.758 | 1.000 | 1.000 | 1.000 |
| 279 | 1.000 | 1.000 | 1.834 | 2.776 | 1.636 |
| 298 | 1000.000 | 0.758 | 1.000 | 1.000 | 1.000 |
| 329 | 1.000 | 1.000 | 1.000 | 0.642 | 1.000 |
| 353 | 1.328 | 1.421 | 2.456 | 1.910 | 2.069 |
| 354 | 1.000 | 1.416 | 2.862 | 2.690 | 1.645 |
| 381 | 1.708 | 2.247 | 1.000 | 0.441 | 0.001 |
| 389 | 1.000 | 1.000 | 1.000 | 0.642 | 1.000 |
| 405 | 1.000 | 1.821 | 1.628 | 2.276 | 2.501 |
| 406 | 1.000 | 1.821 | 1.628 | 2.276 | 2.501 |
| 412 | 1.000 | 1.888 | 1.915 | 2.276 | 1.481 |
| 421 | 3.336 | 1.677 | 2.208 | 1.000 | 1.976 |
| 465 | 1.391 | 1.857 | 1.000 | 0.402 | 1.000 |
| 534 | 1.000 | 1.821 | 1.628 | 2.276 | 2.501 |
| 538 | 1.000 | 1.629 | 2.152 | 1.000 | 1.792 |
| 565 | 1.328 | 1.421 | 2.456 | 1.910 | 2.069 |
| 657 | 1.000 | 1.997 | 2.083 | 3.178 | 3.444 |
| 670 | 1.000 | 1.780 | 1.000 | 2.177 | 2.258 |
| 739 | 1.356 | 0.696 | 1.000 | 1.000 | 1.463 |
| 741 | 2.324 | 1.000 | 2.379 | 1.407 | 2.833 |
| 744 | 2.137 | 1.934 | 2.482 | 2.035 | 3.980 |
| 755 | 0.796 | 1.000 | 1.737 | 1.000 | 2.218 |
| 757 | 2.531 | 3.138 | 0.395 | 1.000 | 1.000 |
| 810 | 0.796 | 1.000 | 1.737 | 1.000 | 2.218 |
| 811 | 0.796 | 1.000 | 1.737 | 1.000 | 2.218 |
| 845 | 1000.000 | 1.984 | 1000.000 | 1.374 | 1.000 |
| 861 | 3.031 | 1.000 | 1.000 | 1.000 | 1.000 |
| 915 | 1.000 | 1.000 | 4.202 | 1.464 | 2.147 |
| 954 | 3.031 | 1.000 | 1.000 | 1.000 | 1.000 |
| 955 | 0.876 | 1.781 | 2.424 | 4.143 | 1.977 |
| 991 | 0.876 | 1.781 | 2.424 | 4.143 | 1.977 |
| 1035 | 1.000 | 1.780 | 1.000 | 2.177 | 2.258 |
| 1049 | 1.356 | 0.696 | 1.000 | 1.000 | 1.463 |
| 1050 | 0.876 | 1.781 | 2.424 | 4.143 | 1.977 |
| 1235 | 1.328 | 1.421 | 2.456 | 1.910 | 2.069 |
| 1292 | 1.000 | 1.000 | 1.992 | 2.144 | 1.615 |
| 1313 | 1.000 | 1.780 | 1.000 | 2.177 | 2.258 |
| 1331 | 1.290 | 1.000 | 1.000 | 1.995 | 2.203 |
| 1334 | 1.000 | 1.416 | 2.862 | 2.690 | 1.645 |
| 1418 | 1.000 | 1.000 | 4.202 | 1.464 | 2.147 |
| 1419 | 1.328 | 1.421 | 2.456 | 1.910 | 2.069 |
| 1420 | 0.816 | 1.000 | 2.196 | 2.446 | 1.518 |
| 1477 | 0.816 | 1.000 | 2.196 | 2.446 | 1.518 |
| 1554 | 1.585 | 1.889 | 2.178 | 1.806 | 1.867 |
| 1579 | 1.243 | 1.679 | 2.228 | 2.333 | 1.774 |
| 1600 | 1.328 | 1.421 | 2.456 | 1.910 | 2.069 |
| 1639 | 0.819 | 1.632 | 2.808 | 5.465 | 2.307 |
| 1657 | 2.810 | 2.638 | 1.976 | 1.491 | 2.955 |
| 1679 | 1.585 | 1.889 | 2.178 | 1.806 | 1.867 |
| 1744 | 1.253 | 1.994 | 1.874 | 3.193 | 2.663 |
| 1847 | 1.559 | 2.762 | 5.043 | 4.135 | 3.753 |
| 1877 | 1.328 | 1.421 | 2.456 | 1.910 | 2.069 |
| 1880 | 1.306 | 1.940 | 2.293 | 3.897 | 1.624 |
| 1889 | 1.328 | 1.421 | 2.456 | 1.910 | 2.069 |
| 2009 | 1.000 | 1.000 | 4.202 | 1.464 | 2.147 |
| 2023 | 1.511 | 1.357 | 1.632 | 1.891 | 1.895 |
| 2029 | 1.243 | 1.679 | 2.228 | 2.333 | 1.774 |
| 2071 | 1.000 | 1.780 | 1.000 | 2.177 | 2.258 |
| 2077 | 1.000 | 1.780 | 1.000 | 2.177 | 2.258 |
| 2103 | 0.573 | 2.678 | 1.000 | 2.507 | 3.278 |
| 2109 | 1.000 | 1.780 | 1.000 | 2.177 | 2.258 |
| 2138 | 7.866 | 1.000 | 1000.000 | 1.719 | 1.000 |
| 2143 | 2.625 | 2.744 | 4.155 | 2.105 | 4.438 |
| 2183 | 1.000 | 2.139 | 3.014 | 3.159 | 3.381 |
| 2185 | 0.796 | 1.000 | 1.737 | 1.000 | 2.218 |
| 2190 | 1.000 | 1.780 | 1.000 | 2.177 | 2.258 |
| 2200 | 1.328 | 1.421 | 2.456 | 1.910 | 2.069 |
| 2206 | 1.489 | 2.750 | 2.910 | 5.049 | 4.006 |
| 2220 | 0.419 | 3.014 | 0.575 | 2.397 | 3.558 |
| 2224 | 1.000 | 1.815 | 2.513 | 3.487 | 2.180 |
| 2249 | 1.328 | 1.421 | 2.456 | 1.910 | 2.069 |
| 2255 | 1.000 | 1.493 | 2.186 | 1.000 | 2.222 |
| 2265 | 1.267 | 3.638 | 1.623 | 5.889 | 3.339 |
| 2267 | 1.746 | 2.363 | 5.515 | 2.674 | 3.637 |
| 2281 | 2.399 | 3.587 | 3.625 | 2.567 | 2.417 |
| 2294 | 1.000 | 1.000 | 4.202 | 1.464 | 2.147 |
| 2300 | 1.000 | 1.000 | 4.202 | 1.464 | 2.147 |
| 2307 | 1.243 | 1.679 | 2.228 | 2.333 | 1.774 |
| 2309 | 0.397 | 1.000 | 1.472 | 5.315 | 2.250 |
| 2313 | 1.000 | 1.939 | 2.505 | 4.525 | 2.674 |
| 2314 | 0.819 | 1.632 | 2.808 | 5.465 | 2.307 |
| 2316 | 1.243 | 1.679 | 2.228 | 2.333 | 1.774 |
| 2327 | 1.295 | 1.658 | 2.836 | 2.766 | 2.873 |
| 2348 | 2.167 | 2.157 | 3.410 | 2.828 | 3.794 |
| 2384 | 1.352 | 1.000 | 2.727 | 0.583 | 1.000 |

TABLE 1

| SEQ ID | CLUSTER | SEQ NAME | ORIENTATION | CLONE ID | LIBRARY |
|---|---|---|---|---|---|
| 1 | 734646 | RTA22200010F.k.10.1.P.Seq | F | M00056481:62 | CH16COP |
| 2 | 400221 | RTA22200001F.a.17.1.P.Seq | F | M00042528:611 | CH15CON |
| 3 | 205329 | RTA22200006F.d.09.2.P.Seq | F | M00056020:410 | CH15CON |
| 4 | 446680 | RTA22200001F.f.07.1.P.Seq | F | M00042693:54 | CH15CON |
| 5 | 1261 | RTA22200021F.j.18.3.P.Seq | F | M00054812:15 | CH17COHLV |
| 6 | 400258 | RTA22200011F.k.23.1.P.Seq | F | M00056617:86 | CH16COP |
| 7 | 450559 | RTA22200005F.e.21.1.P.Seq | F | M00055882:16 | CH15CON |
| 8 | 450959 | RTA22200012F.e.11.1.P.Seq | F | M00056703:46 | CH16COP |
| 9 | 451794 | RTA22200007F.l.16.1.P.Seq | F | M00056247:76 | CH15CON |
| 10 | 415058 | RTA22200020F.d.11.1.P.Seq | F | M00054591:87 | CH17COHLV |
| 11 | 31506 | RTA22200012F.b.08.1.P.Seq | F | M00056670:111 | CH16COP |
| 12 | 417155 | RTA22200002F.f.10.1.P.Seq | F | M00055466:28 | CH15CON |
| 13 | 448925 | RTA22200019F.e.21.1.P.Seq | F | M00043507:45 | CH17COHLV |
| 14 | 11329 | RTA22200006F.d.10.2.P.Seq | F | M00056020:47 | CH15CON |
| 15 | 650422 | RTA22200001F.n.14.1.P.Seq | F | M00042911:83 | CH15CON |
| 16 | 6863 | RTA22200229F.f.13.1.P.Seq | F | M00006967:25 | CH02COH |
| 17 | 449690 | RTA22200002F.g.18.1.P.Seq | F | M00055495:45 | CH15CON |
| 18 | 724616 | RTA22200016F.j.23.1.P.seq | F | M00057236:86 | CH16COP |
| 19 | 549722 | RTA22200025F.m.01.2.P.Seq | F | M00055383:24 | CH17COHLV |
| 20 | 549722 | RTA22200025F.l.24.1.P.Seq | F | M00055383:24 | CH17COHLV |
| 21 | 448110 | RTA22200018F.m.04.1.P.Seq | F | M00043354:31 | CH17COHLV |
| 22 | 515631 | RTA22200010F.j.14.1.P.Seq | F | M00056434:38 | CH16COP |
| 23 | 11881 | RTA22200233F.k.04.1.P.Seq | F | M00008099:78 | CH03MAH |
| 24 | 650856 | RTA22200012F.n.24.1.P.Seq | F | M00056772:14 | CH16COP |
| 25 | 449701 | RTA22200012F.f.21.1.P.Seq | F | M00056710:89 | CH16COP |
| 26 | 651073 | RTA22200007F.l.06.1.P.Seq | F | M00056243:710 | CH15CON |
| 27 | 10340 | RTA22200234F.b.07.1.P.Seq | F | M00022189:23 | CH03MAH |
| 28 | 648310 | RTA22200007F.m.04.1.P.Seq | F | M00056252:88 | CH15CON |
| 29 | 730336 | RTA22200013F.l.02.1.P.Seq | F | M00056879:811 | CH16COP |
| 30 | 3060 | RTA22200018F.b.10.1.P.Seq | F | M00042444:88 | CH17COHLV |
| 31 | 453016 | RTA22200010F.l.06.1.P.Seq | F | M00056485:212 | CH16COP |
| 32 | 508931 | RTA22200024F.i.13.1.P.Seq | F | M00055209:410 | CH17COHLV |
| 33 | 185461 | RTA22200242F.b.06.1.P.Seq | F | M00026975:23 | CH04MAL |
| 34 | 452530 | RTA22200015F.n.11.1.P.Seq | F | M00057131:21 | CH16COP |
| 35 | 448925 | RTA22200026F.d.02.1.P.Seq | F | M00055419:71 | CH17COHLV |
| 36 | 1013 | RTA22200005F.m.06.1.P.Seq | F | M00055945811 | CH15CON |
| 37 | 6545 | RTA22200241F.d.23.1.P.Seq | F | M00026879:410 | CH04MAL |
| 38 | 449891 | RTA22200001F.b.23.1.P.Seq | F | M00042540:85 | CH15CON |
| 39 | 4045 | RTA22200227F.n.06.1.P.Seq | F | M00006740:71 | CH02COH |
| 40 | 404475 | RTA22200002F.b.23.1.P.Seq | F | M00055438:810 | CH15CON |
| 41 | 650297 | RTA22200001F.n.10.1.P.Seq | F | M00042909:74 | CH15CON |
| 42 | 650493 | RTA22200005F.n.03.1.P.Seq | F | M00055959:112 | CH15CON |
| 43 | 644884 | RTA22200007F.k.04.1.P.Seq | F | M00056232:712 | CH15CON |
| 44 | 452212 | RTA22200021F.k.21.3.P.Seq | F | M00054821:311 | CH17COHLV |
| 45 | 402727 | RTA22200010F.n.09.1.P.Seq | F | M00056505:82 | CH16COP |
| 46 | 645194 | RTA22200003F.m.24.1.P.Seq | F | M00055709:79 | CH15CON |
| 47 | 447501 | RTA22200013F.f.14.1.P.Seq | F | M00056839:72 | CH16COP |
| 48 | 556326 | RTA22200003F.o.06.1.P.Seq | F | M00055723:28 | CH15CON |
| 49 | 447035 | RTA22200001F.e.15.1.P.Seq | F | M00042570:82 | CH15CON |
| 50 | 2551 | RTA22200012F.o.07.1.P.Seq | F | M00056774:12 | CH16COP |
| 51 | 736154 | RTA22200010F.i.17.1.P.Seq | F | M00056424:110 | CH16COP |
| 52 | 452028 | RTA22200018F.k.24.1.P.Seq | F | M00043345:33 | CH17COHLV |
| 53 | 447441 | RTA22200001F.m.13.1.P.Seq | F | M00042902:34 | CH15CON |
| 54 | 11028 | RTA22200230F.g.12.1.P.Seq | F | M0007151:211 | CH02COH |
| 55 | 640974 | RTA22200002F.B.07.1.P.Seq | F | M00055429:85 | CH15CON |
| 56 | 555103 | RTA22200022F.m.11.1.P.Seq | F | M00054973:110 | CH17COHLV |
| 57 | 446789 | RTA22200001F.c.12.1.P.Seq | F | M00042547:62 | CH15CON |
| 58 | 644884 | RTA22200007F.k.04.2.P.Seq | F | M00056232:712 | CH15CON |
| 59 | 9029 | RTA22200222F.g.08.1.P.Seq | F | M00003792:111 | CH01COH |
| 60 | 419255 | RTA22200007F.l.14.2.P.Seq | F | M00056246:77 | CH15CON |
| 61 | 4309 | RTA22200225F.j.15.1.P.Seq | F | M00005491:23 | CH02COH |
| 62 | 554069 | RTA22200022F.p.16.1.P.Seq | F | M00055008:28 | CH17COHLV |
| 63 | 4330 | RTA22200227F.i.16.1.P.Seq | F | M00006686:27 | CH02COH |
| 64 | 644903 | RTA22200015F.i.22.1.P.Seq | F | M00057103:511 | CH16COP |
| 65 | 549395 | RTA22200024F.d.04.1.P.Seq | F | M00055163:32 | CH17COHLV |
| 66 | 4974 | RTA22200225F.k.17.1.P.Seq | F | M00005500:53 | CH02COH |
| 67 | 447466 | RTA22200006F.h.03.0.P.Seq | F | M00056053:19 | CH15CON |
| 68 | 645073 | RTA22200004F.o.13.1.P.Seq | F | M00055829:79 | CH15CON |
| 69 | 447978 | RTA22200025F.i.14.1.P.Seq | F | M00055361:81 | CH17COHLV |
| 70 | 607430 | RTA22200004F.p.22.1.P.Seq | F | M00055841:45 | CH15CON |
| 71 | 556198 | RTA22200014F.h.02.2.P.Seq | F | M00056972:65 | CH16COP |
| 72 | 450323 | RTA22200018F.l.23.1.P.Seq | F | M00043352:59 | CH17COHLV |
| 73 | 21205 | RTA22200249F.g.01.1.P.Seq | F | M00027660:53 | CH04MAL |
| 74 | 561109 | RTA22200011F.m.10.1.P.Seq | F | M00056631:43 | CH16COP |
| 75 | 446673 | RTA22200001F.a.14.1.P.Seq | F | M00042525:51 | CH15CON |
| 76 | 456026 | RTA22200004F.m.04.1.P.Seq | F | M00055817:38 | CH15CON |
| 77 | 449142 | RTA22200009F.c.13.2.P.Seq | F | M00042513:112 | CH16COP |
| 78 | 5830 | RTA22200010F.c.23.1.P.Seq | F | M00056370:72 | CH16COP |
| 79 | 554109 | RTA22200026F.g.07.1.P.Seq | F | M00055484:77 | CH17COHLV |
| 80 | 595506 | RTA22200010F.l.16.1.P.Seq | F | M00056491:78 | CH16COP |
| 81 | 453981 | RTA22200010F.p.11.1.P.Seq | F | M00056519:81 | CH16COP |
| 82 | 642461 | RTA22200016F.j.11.1.P.Seq | F | M00057233:54 | CH16COP |
| 83 | 556198 | RTA22200014F.h.02.1.P.Seq | F | M00056972:65 | CH16COP |
| 84 | 2082 | RTA22200009F.g.21.1.P.Seq | F | M00042801:26 | CH16COP |
| 85 | 549435 | RTA22200007F.j.06.1.P.Seq | F | M00056223:73 | CH15CON |
| 86 | 2286 | RTA22200230F.j.03.1.P.Seq | F | M00007177:511 | CH02COH |
| 87 | 2737 | RTA22200023F.d.17.1.P.Seq | F | M00055039:52 | CH17COHLV |
| 88 | 728115 | RTA22200013F.f.13.1.P.Seq | F | M00056839:71 | CH16COP |
| 89 | 650856 | RTA22200012F.o.01.1.P.Seq | F | M00056772:14 | CH16COP |
| 90 | 650476 | RTA22200005F.g.21.1.P.Seq | F | M00055891:14 | CH15CON |
| 91 | 535208 | RTA22200005F.b.02.1.P.Seq | F | M00055852:17 | CH15CON |
| 92 | 733849 | RTA22200011F.m.24.1.P.Seq | F | M00056638:48 | CH16COP |
| 93 | 447978 | RTA22200009F.g.19.1.P.Seq | F | M00042800:13 | CH16COP |
| 94 | 729483 | RTA22200012F.i.12.1.P.Seq | F | M00056728:45 | CH16COP |
| 95 | 12018 | RTA22200249F.e.19.1.P.Seq | F | M00027641:11 | CH04MAL |
| 96 | 4747 | RTA22200227F.d.18.1.P.Seq | F | M00006630:311 | CH02COH |
| 97 | 4747 | RTA22200225F.a.23.1.P.Seq | F | M00005415:612 | CH02COH |
| 98 | 185577 | RTA22200240F.i.02.1.P.Seq | F | M00023409:78 | CH04MAL |
| 99 | 4126 | RTA22200231F.m.17.1.P.Seq | F | M00007990:43 | CH03MAH |
| 100 | 11456 | RTA22200226F.l.08.1.P.Seq | F | M00005765:67 | CH02COH |
| 101 | 729851 | RTA22200010F.o.03.1.P.Seq | F | M00056508:210 | CH16COP |
| 102 | 449849 | RTA22200019F.b.07.1.P.Seq | F | M00043394:26 | CH17COHLV |
| 103 | 2490 | RTA22200237F.e.17.1.P Seq | F | M00022720:111 | CH03MAH |
| 104 | 549041 | RTA22200021F.h.21.3.P.Seq | F | M00054779:67 | CH17COHLV |
| 105 | 11881 | RTA22200237F.i.01.1.P.Seq | F | M00022750:17 | CH03MAH |
| 106 | 724296 | RTA22200014F.o.10.1.P.Seq | F | M00057025:18 | CH16COP |
| 107 | 726173 | RTA22200014F.o.01.1.P.Seq | F | M00057023:89 | CH16COP |
| 108 | 2423 | RTA22200236F.h.20.1.P.Seq | F | M00022641:62 | CH03MAH |
| 109 | 556250 | RTA22200014F.l.09.1.P.Seq | F | M00057005:31 | CH16COP |
| 110 | 643594 | RTA22200005F.b.01.1.P.Seq | F | M00055851:612 | CH15CON |
| 111 | 11881 | RTA22200237F.h.24.1.P.Seq | F | M00022750:17 | CH03MAH |
| 112 | 7436 | RTA22200237F.j.11.1.P.Seq | F | M00022791:611 | CH03MAH |
| 113 | 2110 | RTA22200235F.a.19.1.P.Seq | F | M00022415:26 | CH03MAH |
| 114 | 10340 | RTA22200235F.e.13.1.P.Seq | F | M00022457:75 | CH03MAH |
| 115 | 643594 | RTA22200005F.a.24.1.P.Seq | F | M00055851:612 | CH15CON |
| 116 | 447035 | RTA22200005F.d.11.1.P.Seq | F | M00055872:412 | CH15CON |
| 117 | 402707 | RTA22200007F.c.21.1.P.Seq | F | M00056160:18 | CH15CON |
| 118 | 645799 | RTA22200014F.d.22.1.P.Seq | F | M00056952:84 | CH16COP |
| 119 | 171511 | RTA22200014F.c.05.1.P.Seq | F | M00056939:22 | CH16COP |
| 120 | 451607 | RTA22200014F.h.15.2.P.Seq | F | M00056976:610 | CH16COP |
| 121 | 3138 | RTA22200242F.k.08.1.P.Seq | F | M00027039:59 | CH04MAL |
| 122 | 2988 | RTA22200229F.h.11.1.P.Seq | F | M00006987:711 | CH02COH |
| 123 | 447327 | RTA22200014F.g.22.1.P.Seq | F | M00056969:21 | CH16COP |
| 124 | 561734 | RTA22200014F.f.10.1.P.Seq | F | M00056961:712 | CH16COP |
| 125 | 454999 | RTA22200009F.a.13.2.P.Seq | F | M00042432:810 | CH16COP |
| 126 | 185652 | RTA22200242F.i.12.1.P.Seq | F | M00027028:37 | CH04MAL |
| 127 | 6725 | RTA22200232F.d.17.1.P.Seq | F | M00021947:36 | CH03MAH |
| 128 | 726644 | RTA22200013F.i.19.1.P.Seq | F | M00056864:89 | CH16COP |
| 129 | 11012 | RTA22200227F.n.21.1.P.Seq | F | M00006745:12 | CH02COH |
| 130 | 726377 | RTA22200013F.i.03.1.P.Seq | F | M00056860:612 | CH16COP |
| 131 | 735326 | RTA22200013F.l.19.1.P.Seq | F | M000568860:311 | CH16COP |
| 132 | 650845 | RTA22200013F.l.13.1.P.Seq | F | M00056884:36 | CH16COP |
| 133 | 9048 | RTA22200222F.l.18.1.P.Seq | F | M00004101:81 | CH01COH |
| 134 | 732254 | RTA22200012F.b.15.1.P.Seq | F | M00056674:84 | CH16COP |
| 135 | 452052 | RTA22200013F.i.16.1.P.Seq | F | M00056863:53 | CH16COP |
| 136 | 554079 | RTA22200012F.j.17.1.P.Seq | F | M00056735:28 | CH16COP |
| 137 | 9049 | RTA22200222F.i.05.1.P.Seq | F | M00003948:212 | CH01COH |
| 138 | 1307 | RTA22200244F.n.16.1.P.Seq | F | M00027222:39 | CH04MAL |
| 139 | 139730 | RTA22200242F.g.11.1.P.Seq | F | M00027016:76 | CH04MAL |
| 140 | 7750 | RTA22200241F.f.24.1.P.Seq | F | M00026899:711 | CH04MAL |
| 141 | 8050 | RTA22200227F.p.20.1.P.Seq | F | M00006761:49 | CH02COH |
| 142 | 725222 | RTA22200013F.k.24.1.P.Seq | F | M00056879:55 | CH16COP |
| 143 | 3275 | RTA22200235F.j.22.2.P.Seq | F | M00022516:59 | CH03MAH |
| 144 | 7424 | RTA22200235F.c.12.1.P.Seq | F | M00022430:44 | CH03MAH |
| 145 | 8953 | RTA22200241F.c.05.1.P.Seq | F | M00026866:88 | CH04MAL |
| 146 | 8966 | RTA22200243F.c.04.1.P.Seq | F | M00027088:86 | CH04MAL |
| 147 | 530883 | RTA22200013F.i.22.1.P.Seq | F | M00056866:55 | CH16COP |
| 148 | 6725 | RTA22200238F.l.09.1.P.Seq | F | M00022973:78 | CH03MAH |
| 149 | 4439 | RTA22200222F.m.24.1.P.Seq | F | M00004167:411 | CH01COH |
| 150 | 648472 | RTA22200012F.g.07.1.P.Seq | F | M00056712:17 | CH16COP |
| 151 | 735346 | RTA22200011F.l.03.1.P.Seq | F | M00056618:22 | CH16COP |
| 152 | 732121 | RTA22200011F.j.05.1.P.Seq | F | M00056600:87 | CH16COP |
| 153 | 650337 | RTA22200005F.h.15.1.P.Seq | F | M00055900.25 | CH15CON |
| 154 | 533588 | RTA22200005F.p.05.i.P.Seq | F | M00055981:17 | CH15CON |
| 155 | 649667 | RTA22200007F.p.20.1.P.Seq | F | M00056290:82 | CH15CON |
| 156 | 394436 | RTA22200015F.p.07.1.P.Seq | F | M00057145:45 | CH16COP |
| 157 | 649354 | RTA22200007F.h.13.1.P.Seq | F | M00056210:53 | CH15CON |
| 158 | 2022 | RTA22200240F.e.10.1.P.Seq | F | M00023347:312 | CH04MAL |
| 159 | 561359 | RTA22200003F.m.08.1.P.Seq | F | M00055703:26 | CH15CON |
| 160 | 76O7 | RTA22200225F.m.20.1.P.Seq | F | M00005520:512 | CH02COH |
| 161 | 7750 | RTA22200242F.f.06.1.P.Seq | F | M00027006:81 | CH04MAL |
| 162 | 410554 | RTA22200012F.i.21.1.P.Seq | F | M00056729:812 | CH16COP |
| 163 | 2315 | RTA22200230F.e.24.1.P.Seq | F | M00007135:211 | CH02COH |
| 164 | 561734 | RTA22200014F.f.10.2.P.Seq | F | M00056961:712 | CH16COP |
| 165 | 4420 | RTA22200229F.f.02.1.P.Seq | F | M00006964:111 | CH02COH |
| 166 | 559663 | RTA22200012F.d.17.1.P.Seq | F | M00056697:53 | CH16COP |
| 167 | 7082 | RTA22200235F.p.01.2.P.Seq | F | M00022565:15 | CH03MAH |
| 168 | 2315 | RTA22200230F.f.01.1.P.Seq | F | M00007135:211 | CH02COH |
| 169 | 650472 | RTA22200012F.j.21.1.P.Seq | F | M00056737:77 | CH16COP |
| 170 | 6482 | RTA22200230F.a.10.1.P.Seq | F | M00007096:51 | CH02COH |
| 171 | 4584 | RTA22200230F.a.09.1.P.Seq | F | M00007096:52 | CH02COH |
| 172 | 453846 | RTA22200012F.b.14.1.P.Seq | F | M00056674:55 | CH16COP |
| 173 | 650820 | RTA22200011F.p.05.1.P.Seq | F | M0005665:83 | CH16COP |
| 174 | 642906 | RTA22200005F.g.06.1.P.Seq | F | M00055887:36 | CH15CON |
| 175 | 448805 | RTA22200005F.i.23.1.P.Seq | F | M00055912:35 | CH15CON |
| 176 | 649667 | RTA22200006F.k.18.2.P.Seq | F | M00056082:66 | CH15CON |
| 177 | 735786 | RTA22200012F.m.12.1.P.Seq | F | M00056758:35 | CH16COP |
| 178 | 121457 | RTA22200012F.p.18.1.P.Seq | F | M00056785:68 | CH16COP |
| 179 | 372960 | RTA22200012F.m.06.1.P.Seq | F | M00056756:28 | CH16COP |
| 180 | 120049 | RTA22200012F.j.10.1.P.Seq | F | M00056733:49 | CH16COP |
| 181 | 648996 | RTA22200006F.p.20.2.P.Seq | F | M00056136:11 | CH15CON |
| 182 | 3765 | RTA22200226F.a.16.1.P.Seq | F | M00005589:67 | CH02COH |
| 183 | 462642 | RTA22200008F.e.08.1.P.Seq | F | M00056342:75 | CH15CON |
| 184 | 727181 | RTA22200016F.f.18.1.P.Seq | F | M00057208:12 | CH16COP |
| 185 | 649259 | RTA22200006F.k.06.2.P.Seq | F | M00056079:412 | CH15CON |
| 186 | 649717 | RTA22200007F.e.08.1.P.Seq | F | M00056180:89 | CH15CON |
| 187 | 736860 | RTA22200009F.p.24.1.P.Seq | F | M00056351:46 | CH16COP |
| 188 | 729175 | RTA22200012F.o.05.1.P.Seq | F | M00056773:811 | CH16COP |
| 189 | 642906 | RTA22200005F.f.14.1.P.Seq | F | M00055884:15 | CH15CON |
| 190 | 4420 | RTA22200232F.l.13.1.P.Seq | F | M00022123:45 | CH03MAH |
| 191 | 2420 | RTA22200226F.c.12.1.P.Seq | F | M00005619:19 | CH02COH |
| 192 | 648109 | RTA22200015F.o.03.1.P.Seq | F | M00057135:84 | CH16COP |
| 193 | 2334 | RTA22200011F.p.20.1.P.Seq | F | M00056661:19 | CH16COP |
| 194 | 639705 | RTA22200003F.o.14.1.P.Seq | F | M00055724:49 | CH15CON |
| 195 | 551907 | RTA22200003F.n.12.1.P.Seq | F | M00055717:64 | CH15CON |
| 196 | 561382 | RTA22200003F.m.20.1.P.Seq | F | M00055706:71 | CH15CON |
| 197 | 595506 | RTA22200022F.a.01.1.P.Seq | F | M00054866:77 | CH17COHLV |
| 198 | 499424 | RTA22200013F.f.16.1.P.Seq | F | M00056839:61 | CH16COP |
| 199 | 735477 | RTA22200016F.f.08.1.P.Seq | F | M00057203:56 | CH16COP |
| 200 | 734370 | RTA22200013F.g.21.1.P.Seq | F | M00056848:37 | CH16COP |
| 201 | 779 | RTA22200230F.c.07.1.P.Seq | F | M00007112:112 | CH02COH |
| 202 | 649143 | RTA22200007F.o.24.1.P.Seq | F | M00056283:52 | CH15CON |
| 203 | 489 | RTA22200012F.m.16.1.P.Seq | F | M00056759:611 | CH16COP |
| 204 | 2994 | RTA22200229F.k.24.1.P.Seq | F | M00007028:34 | CH02COH |
| 205 | 2994 | RTA22200229F.l.01.1.P.Seq | F | M00007028:34 | CH02COH |
| 206 | 11147 | RTA22200227F.g.12.1.P.Seq | F | M00006664:29 | CH02COH |
| 207 | 549395 | RTA22200021F.d.19.2.P.Seq | F | M00054745:13 | CH17COHLV |
| 208 | 559806 | RTA22200022F.h.11.1.P.Seq | F | M00054937:63 | CH17COHLV |
| 209 | 452238 | RTA22200009F.k.17.2.P.Seq | F | M00042838:511 | CH16COP |
| 210 | 225914 | RTA22200023F.j.18.1.P.Seq | F | M00055075:85 | CH17COHLV |
| 211 | 463480 | RTA22200022F.p.18.1.P.Seq | F | M00055008:29 | CH17COHLV |
| 212 | 184725 | RTA22200007F.o.17.1.P.Seq | F | M00056281:54 | CH15CON |
| 213 | 557401 | RTA22200023F.g.22.1.P.Seq | F | M00055056:26 | CH17COHLV |
| 214 | 455155 | RTA22200003F.f.04.1.P.Seq | F | M00055633:711 | CH15CON |
| 215 | 551117 | RTA22200023F.p.03.1.P.Seq | F | M00055131:210 | CH17COHLV |
| 216 | 7659 | RTA22200231F.g.16.1.P.Seq | F | M00007964:71 | CH03MAH |
| 217 | 729295 | RTA22200017F.b.14.1.P.Seq | F | M00057306:87 | CH16COP |
| 218 | 450429 | RTA22200007F.f.20.1.P.Seq | F | M00056195:38 | CH15CON |
| 219 | 450148 | RTA22200022F.f.21.1.P.Seq | F | M00054927:58 | CH17COHLV |
| 220 | 380412 | RTA22200006F.c.23.2.P.Seq | F | M00056016:46 | CH15CON |
| 221 | 446614 | RTA22200001F.e.01.1.P.Seq | F | M00042563:52 | CH15CON |
| 222 | 555911 | RTA22200023F.f.21.i.P.Seq | F | M00055050:74 | CH17COHLV |
| 223 | 450828 | RTA22200022F.g.23.1.P.Seq | F | M00054935:74 | CH17COHLV |
| 224 | 28 | RTA22200006F.a.17.2.P.Seq | F | M00055999:710 | CH15CON |
| 225 | 446450 | RTA22200001F.h.15.1.P.Seq | F | M00042717:44 | CH15CON |
| 226 | 452026 | RTA22200010F.g.18.1.P.Seq | F | M0005611:54 | CH16COP |
| 227 | 643594 | RTA22200006F.a.18.2.P.Seq | F | M00055999:76 | CH15CON |
| 228 | 1905 | RTA22200012F.o.14.1.P.Seq | F | M00056775:38 | CH16COP |
| 229 | 651073 | RTA22200007F.l.06.2.P.Seq | F | M00056243:710 | CH15CON |
| 230 | 553705 | RTA22200006F.a.23.2.P.Seq | F | M00056001:27 | CH15CON |
| 231 | 521840 | RTA22200004F.j.15.1.P.Seq | F | M00055802:84 | CH15CON |
| 232 | 648689 | RTA22200006F.o.07.2.P.Seq | F | M00056111:82 | CH15CON |
| 233 | 447858 | RTA22200022F.d.10.1.P.Seq | F | M00054895:49 | CH17COHLV |
| 234 | 556198 | RTA22200010F.d.10.1.P.Seq | F | M00056374:82 | CH16COP |
| 235 | 394436 | RTA22200003F.i.09.1.P.Seq | F | M00055662:14 | CH15CON |
| 236 | 639651 | RTA22200003F.g.12.1.P.Seq | F | M00055647:24 | CH15CON |
| 237 | 499424 | RTA22200010F.d.18.1.P.Seq | F | M00056382:82 | CH16COP |
| 238 | 468109 | RTA22200001F.p.12.1.P.Seq | F | M00054915:57 | CH15CON |
| 239 | 185701 | RTA22200248F.g.23.1.P.Seq | F | M00027561:34 | CH04MAL |
| 240 | 451811 | RTA22200006F.h.04.2.P.Seq | F | M00056053:412 | CH15CON |
| 241 | 730670 | RTA22200009F.m.14.1.P.Seq | F | M00042850:34 | CH16COP |
| 242 | 172013 | RTA22200021F.l.15.3.P.Seq | F | M00054826:310 | CH17COHLV |
| 243 | 449142 | RTA22200001F.f.10.1.P.Seq | F | M00042694:52 | CH15CON |
| 244 | 446964 | RTA22200001F.h.23.1.P.Seq | F | M00042721:77 | CH15CON |
| 245 | 414739 | RTA22200022F.i.16.1.P.Seq | F | M00054945:77 | CH17COHLV |
| 246 | 641124 | RTA22200004F.k.12.1.P.Seq | F | M00055805:37 | CH15CON |
| 247 | 555702 | RTA22200022F.i.10.1.P.Seq | F | M00054949:53 | CH17COHLV |
| 248 | 549435 | RTA22200019F.p.14.1.P.Seq | F | M00054561:56 | CH17COHLV |
| 249 | 643954 | RTA22200002F.d.19.1.P.Seq | F | M00055451:711 | CH15CON |
| 250 | 5984 | RTA22200026F.d.05.1.P.Seq | F | M00055420:56 | CH17COHLV |
| 251 | 560526 | RTA22200022F.f.10.1.P.Seq | F | M00054910:76 | CH17COHLV |
| 252 | 411113 | RTA22200023F.a.16.1.P.Seq | F | M00055017:111 | CH17COHLV |
| 253 | 7607 | RTA22200004F.b.13.1.P.Seq | F | M00055744:69 | CH15CON |
| 254 | 559409 | RTA22200019F.o.07.1.P.Seq | F | M00054551:73 | CH17COHLV |
| 255 | 650053 | RTA22200002F.p.11.1.P.Seq | F | M00055560:62 | CH15CON |
| 256 | 448511 | RTA22200009F.f.03.1.P.Seq | F | M00042777:46 | CH16COP |
| 257 | 642142 | RTA22200016F.o.20.1.P.Seq | F | M00057277:510 | CH16COP |
| 258 | 470462 | RTA22200006F.b.14.2.P.Seq | F | M00056005:34 | CH15CON |
| 259 | 431601 | RTA22200004F.d.07.1.P.Seq | F | M00055763:56 | CH15CON |
| 260 | 421431 | RTA22200001F.g.16.1.P.Seq | F | M00042704:52 | CH15CON |
| 261 | 284586 | RTA22200002F.d.17.1.P.Seq | F | M00055451:67 | CH15CON |
| 262 | 556198 | RTA22200022F.l.06.1.P.Seq | F | M00054963:84 | CH17COHLV |
| 263 | 431601 | RTA22200004F.j.04.1.P.Seq | F | M00055800:48 | CH15CON |
| 264 | 449891 | RTA22200002F.n.21.1.P.Seq | F | M00055548:64 | CH15CON |
| 265 | 556561 | RTA22200022F.p.01.1.P.Seq | F | M00054997:212 | CH17COHLV |
| 266 | 554188 | RTA22200022F.o.20.1.P.Seq | F | M00054996:39 | CH17COHLV |
| 267 | 3247 | RTA22200006F.a.22.2.P.Seq | F | M00056001:26 | CH15CON |
| 268 | 546705 | RTA22200022F.k.20.1.P.Seq | F | M00054959:311 | CH17COHLV |
| 269 | 560984 | RTA22200022F.p.02.1.P.Seq | F | M00054997:83 | CH17COHLV |
| 270 | 455820 | RTA22200006F.g.03.2.P.Seq | F | M00056045:81 | CH17COHLV |
| 271 | 643129 | RTA22200006F.n.12.2.P.Seq | F | M06056103:412 | CH15CON |
| 272 | 454653 | RTA22200023F.o.20.1.P.Seq | F | M00055128:210 | CH17COHLV |
| 273 | 456549 | RTA22200002F.k.01.1.P.Seq | F | M00055522:32 | CH15CON |
| 274 | 454806 | RTA22200004F.n.04.1.P.Seq | F | M00055822:84 | CH15CON |
| 275 | 724296 | RTA22200014F.o.10.2.P.Seq | F | M00057025:18 | CH16COP |
| 276 | 559280 | RTA22200015F.j.19.1.P.Seq | F | M00057106:26 | CH16COP |
| 277 | 171511 | RTA22200014F.c.05.2.P.Seq | F | M00056939:22 | CH16COP |
| 278 | 644242 | RTA22200002F.o.14.1.P.Seq | F | M00055553:84 | CH15CON |
| 279 | 734370 | RTA22200016F.j.07.1.P.Seq | F | M00057232:46 | CH16COP |
| 280 | 639459 | RTA22200002F.i.18.1.P.Seq | F | M00055512:76 | CH15CON |
| 281 | 641679 | RTA22200003F.d.20.1.P.Seq | F | M00055613:52 | CH15CON |
| 282 | 644611 | RTA22200002F.i.10.1.P.Seq | F | M00055509:89 | CH15CON |
| 283 | 550038 | RTA22200021F.i.16.3.P.Seq | F | M00054802:72 | CH17COHLV |
| 284 | 452567 | RTA22200002F.J.12.1.P.Seq | F | M00055519:36 | CH15CON |
| 285 | 411113 | RTA22200024F.f.03.1.P.Seq | F | M00055185:21 | CH17COHLV |
| 286 | 650749 | RTA22200002F.d.18.1.P.Seq | F | M00055451:611 | CH15CON |
| 287 | 558899 | RTA22200026F.d.12.1.P.Seq | F | M00055421:44 | CH17COHLV |
| 288 | 452986 | RTA22200002F.a.13.1.P.Seq | F | M00055426:22 | CH15CON |
| 289 | 393197 | RTA22200015F.k.01.1.P.Seq | F | M00057108:59 | CH16COP |
| 290 | 499424 | RTA22200024F.e.10.1.P.Seq | F | M00055179:42 | CH17COHLV |
| 291 | 21669 | RTA22200025F.m.07.2.P.Seq | F | M00055384:13 | CH17COHLV |
| 292 | 640590 | RTA22200004F.h.21.1.P.Seq | F | M00055794:711 | CH15CON |
| 293 | 549936 | RTA22200024F.c.10.1.P.Seq | F | M00055157:311 | CH17COHLV |
| 294 | 448770 | RTA22200016F.c.17.1.P.Seq | F | M00057174:712 | CH16COP |
| 295 | 559280 | RTA22200015F.h.14.1.P.Seq | F | M00057093:69 | CH16COP |
| 296 | 648934 | RTA22200003F.c.10.1.P.Seq | F | M00055591:81 | CH15CON |
| 297 | 452685 | RTA22200004F.p.06.1.P.Seq | F | M00055838:412 | CH15CON |
| 298 | 456549 | RTA22200002F.j.24.1.P.Seq | F | M00055522:32 | CH15CON |
| 299 | 446614 | RTA22200001F.d.24.1.P.Seq | F | M00042563:52 | CH15CON |
| 300 | 559280 | RTA22200024F.d.18.1.P.Seq | F | M00055170:52 | CH17COHLV |
| 301 | 446673 | RTA22200015F.c.01.1.P.Seq | F | M00057055:78 | CH16COP |
| 302 | 562550 | RTA22200004F.n.17.1.P.Seq | F | M00055826:64 | CH15CON |
| 303 | 467288 | RTA22200002F.h.16.1.P.Seq | F | M00055500:25 | CH15CON |
| 304 | 463824 | RTA22200004F.n.10.1.P.Seq | F | M00055823:43 | CH15CON |
| 305 | 393197 | RTA22200015F.j.24.1.P.Seq | F | M00057108:59 | CH16COP |
| 306 | 407077 | RTA22200015F.j.22.1.P.Seq | F | M00057108:64 | CH16COP |
| 307 | 499424 | RTA22200018F.h.13.1.P.Seq | F | M00043317:81 | CH17COHLV |
| 308 | 554500 | RTA22200021F.o.20.2.P.Seq | F | M00054857:512 | CH17COHLV |
| 309 | 730143 | RTA22200015F.k.14.1.P.Seq | F | M00057112:29 | CH16COP |
| 310 | 595506 | RTA22200015F.c.21.1.P.Seq | F | M00057061:44 | CH16COP |
| 311 | 2334 | RTA22200016F.k.13.1.P.Seq | F | M00057242:85 | CH16COP |
| 312 | 647444 | RTA22200002F.k.22.1.P.Seq | F | M00055527:711 | CH15CON |
| 313 | 380291 | RTA22200008F.a.03.1.P Seq | F | M00056292:55 | CH15CON |
| 314 | 644849 | RTA22200016F.a.22.1.P.Seq | F | M00057162:37 | CH16COP |
| 315 | 449457 | RTA22200018F.g.23.1.P.Seq | F | M00043314:84 | CH17COHLV |
| 316 | 446673 | RTA22200015F.b.24.1.P.Seq | F | M00057055:78 | CH16COP |
| 317 | 549069 | RTA22200020F.e.05.1.P.Seq | F | M00054596:27 | CH17COHLV |
| 318 | 728884 | RTA22200015F.d.02.1.P.Seq | F | M00057063:38 | CH16COP |
| 319 | 415058 | RTA22200020F.f.03.1.P.Seq | F | M00054605:41 | CH17COHLV |
| 320 | 553955 | RTA22200020F.e.20.1.P.Seq | F | M00054602:42 | CH17COHLV |
| 321 | 455820 | RTA22200018F.i.16.1.P.Seq | F | M00043323:44 | CH17COHLV |
| 322 | 549617 | RTA22200020F.f.20.1.P.Seq | F | M00054611:62 | CH17COHLV |
| 323 | 449831 | RTA22200018F.d.22.1.P.Seq | F | M00042518:16 | CH17COHLV |
| 324 | 451580 | RTA22200018F.b.09.1.P.Seq | F | M00042444:75 | CH17COHLV |
| 325 | 558899 | RTA22200025F.f.09.1.P.Seq | F | M00055325:212 | CH17COHLV |
| 326 | 562292 | RTA22200025F.j.10.1.P.Seq | F | M00055368:26 | CH17COHLV |
| 327 | 5830 | RTA22200011F.d.11.1.P.Seq | F | M00056552:110 | CH16COP |
| 328 | 8953 | RTA22200248F.p.09.1.P.Seq | F | M00027608:87 | CH04MAL |
| 329 | 8012 | RTA22200233F.i.02.1.P.Seq | F | M00008089:59 | CH03MAH |
| 330 | 185718 | RTA22200250F.f.15.1.P.Seq | F | M00027829:42 | CH04MAL |
| 331 | 729851 | RTA22200011F.b.18.1.P.Seq | F | M00056537:85 | CH16COP |
| 332 | 185597 | RTA22200249F.a.20.1.P.Seq | F | M00027616:712 | CH04MAL |
| 333 | 9887 | RTA22200234F.h.14.1.P.Seq | F | M00022253:53 | CH03MAH |
| 334 | 725825 | RTA22200011F.c.16.1.P.Seq | F | M00056547:34 | CH16COP |
| 335 | 6545 | RTA22200249F.e.12.1.P.Seq | F | M00027639:511 | CH04MAL |
| 336 | 21205 | RTA22200249F.f.24.1.P.Seq | F | M00027660:53 | CH04MAL |
| 337 | 8867 | RTA22200234F.j.23.1.P.Seq | F | M00022280:711 | CH03MAH |
| 338 | 729295 | RTA22200010F.m.09.1.P.Seq | F | M00056499:65 | CH16COP |
| 339 | 730430 | RTA22200010F.h.08.1.P.Seq | F | M00056416:212 | CH16COP |
| 340 | 7072 | RTA22200233F.h.20.1.P.Seq | F | M00008085:39 | CH03MAH |
| 341 | 730533 | RTA22200010F.n.17.1.P.Seq | F | M00056506:712 | CH16COP |
| 342 | 9121 | RTA22200224F.g.11.1.P.Seq | F | M00005312:410 | CH02COH |
| 343 | 11131 | RTA22200224F.c.08.1.P.Seq | F | M00004852:14 | CH02COH |
| 344 | 640116 | RTA22200010F.n.01.1.P.Seq | F | M00056503:711 | CH16COP |
| 345 | 730282 | RTA22200010F.k.18.1.P.Seq | F | M00056483:66 | CH16COP |
| 346 | 550571 | RTA22200019F.h.11.1.P.Seq | F | M00054502:52 | CH17COHLV |
| 347 | 1183 | RTA22200248F.c.22.1.P.Seq | F | M00027527:74 | CH04MAL |
| 348 | 449437 | RTA22200019F.g.09.1.P.Seq | F | M00054494:51 | CH17COHLV |
| 349 | 8966 | RTA22200242F.d.21.1.P.Seq | F | M00026994:17 | CH04MAL |
| 350 | 6134 | RTA22200006F.p.04.2.P.Seq | F | M00056125:49 | CH15CON |
| 351 | 95700 | RTA22200241F.f.09.1.P.Seq | F | M00026896:510 | CH04MAL |
| 352 | 7066 | RTA22200229F.a.23.1.P.Seq | F | M00006928:14 | CH02COH |
| 353 | 648310 | RTA22200007F.a.23.1.P.Seq | F | M00056143:59 | CH15CON |
| 354 | 730059 | RTA22200013F.k.11.1.P.Seq | F | M00056874:45 | CH16COP |
| 355 | 736014 | RTA22200012F.f.11.1.P.Seq | F | M00056708:411 | CH16COP |
| 356 | 646577 | RTA22200012F.d.07.1.P.Seq | F | M00056693:38 | CH16COP |
| 357 | 732254 | RTA22200014F.g.09.1.P.Seq | F | M00056967:57 | CH16COP |
| 358 | 7037 | RTA22200229F.b.01.1.P.Seq | F | M00006928:41 | CH02COH |
| 359 | 7037 | RTA22200229F.a.24.1.P.Seq | F | M00006928:41 | CH02COH |
| 360 | 6937 | RTA22200232F.b.06.1.P.Seq | F | M00021864:57 | CH03MAH |
| 361 | 7572 | RTA22200228F.k.06.2.P.Seq | F | M00006867:612 | CH02COH |
| 362 | 388085 | RTA22200005F.c.21.1.P.Seq | F | M00055868:43 | CH15CON |
| 363 | 2676 | RTA22200227F.p.19.1.P.Seq | F | M00006761:35 | CH02COH |
| 364 | 639240 | RTA22200007F.p.19.1.P.Seq | F | M00056238:57 | CH15CON |
| 365 | 650472 | RTA22200012F.e.19.1.P.Seq | F | M00056705:57 | CH16COP |
| 366 | 727789 | RTA22200012F.g.19.1.P.Seq | F | M00056715:31 | CH16COP |
| 367 | 2495 | RTA22200238F.e.03.1.P.Seq | F | M00022895:211 | CH03MAH |
| 368 | 732254 | RTA22200014F.g.09.2.P.Seq | F | M00056967:57 | CH16COP |
| 369 | 5268 | RTA22200225F.m.13.1.P.Seq | F | M00005517:64 | CH02COH |
| 370 | 11881 | RTA22200238F.e.09.1.P.Seq | F | M00022897:83 | CH03MAH |
| 371 | 448677 | RTA22200009F.b.05.2.P.Seq | F | M00042440:55 | CH16COP |
| 372 | 1876 | RTA22200235F.i.14.2.P.Seq | F | M00022496:64 | CH03MAH |
| 373 | 3441 | RTA22200233F.e.23.1.P.Seq | F | M00008065:62 | CH03MAH |
| 374 | 726134 | RTA22200011F.l.05.1.P.Seq | F | M00056618:611 | CH16COP |
| 375 | 9048 | RTA22200222F.g.12.1.P.Seq | F | M00003804:19 | CH01COH |
| 376 | 26489 | RTA22200242F.k.07.1.P.Seq | F | M00027039:66 | CH04MAL |
| 377 | 644205 | RTA22200007F.c.24.1.P.Seq | F | M00056162:68 | CH15CON |
| 378 | 468689 | RTA22200006F.f.04.2.P.Seq | F | M00056036:26 | CH15CON |
| 379 | 638971 | RTA22200012F.k.19.1.P.Seq | F | M00056746:12 | CH16COP |
| 380 | 10274 | RTA22200241F.l.17.1.P.Seq | F | M00026936:312 | CH04MAL |
| 381 | 6725 | RTA22200232F.f.22.1.P.Seq | F | M00022013:85 | CH03MAH |
| 382 | 2488 | RTA22200012F.l.21.1.P.Seq | F | M00056754:49 | CH16COP |
| 383 | 8366 | RTA22200244F.c.08.1.P.Seq | F | M00027173:48 | CH04MAL |
| 384 | 502683 | RTA22200007F.a.09.1.P.Seq | F | M00056139:71 | CH15CON |
| 385 | 450914 | RTA22200012F.k.22.1.P.Seq | F | M00056747:45 | CH16COP |
| 386 | 21205 | RTA22200243F.k.21.1.P.Seq | F | M00027140:311 | CH04MAL |
| 387 | 644205 | RTA22200007F.d.01.1.P.Seq | F | M00056162:68 | CH15CON |
| 388 | 5268 | RTA22200225F.m.05.1.P.Seq | F | M00005513:69 | CH02COH |
| 389 | 8012 | RTA22200232F.n.17.1.P.Seq | F | M00022148:16 | CH03MAH |
| 390 | 11270 | RTA22200227F.m.18.1.P.Seq | F | M00006734:18 | CH02COH |
| 391 | 10924 | RTA22200237F.h.04.1.P.Seq | F | M00022738:46 | CH03MAH |
| 392 | 11619 | RTA22200241F.g.14.1.P.Seq | F | M00026902:74 | CH04MAL |
| 393 | 3650 | RTA22200236F.e.11.1.P.Seq | F | M00022617:32 | CH03MAH |
| 394 | 1655 | RTA22200222F.e.14.1.P.Seq | F | M00001637:49 | CH01COH |
| 395 | 3275 | RTA22200238F.b.21.1.P.Seq | F | M00022876:25 | CH03MAH |
| 396 | 3355 | RTA22200238F.c.05.1.P.Seq | F | M00022880:79 | CH03MAH |
| 397 | 2078 | RTA22200235F.g.18.1.P.Seq | F | M00022473:26 | CH03MAH |
| 398 | 4809 | RTA22200222F.g.09.1.P.Seq | F | M00003794:47 | CH01COH |
| 399 | 6402 | RTA22200236F.l.15.1.P.Seq | F | M00022660:24 | CH03MAL |
| 400 | 555244 | RTA22200023F.e.03.1.P.Seq | F | M00055042:21 | CH17COHLV |
| 401 | 548965 | RTA22200012F.g.16.1.P.Seq | F | M00056715:410 | CH16COP |
| 402 | 4747 | RTA22200227F.m.12.1.P.Seq | F | M00006731:38 | CH02COH |
| 403 | 40208 | RTA22200241F.n.21.1.P.Seq | F | M00026950:81 | CH04MAL |
| 404 | 14596 | RTA22200241F.d.18.1.P.Seq | F | M00026879:22 | CH04MAL |
| 405 | 7110 | RTA22200232F.n.01.1.P.Seq | F | M00022143:41 | CH03MAH |
| 406 | 7110 | RTA22200232F.m.24.1.P.Seq | F | M00022143:41 | CH03MAH |
| 407 | 6592 | RTA22200238F.p.20.1.P.Seq | F | M00023029:56 | CH03MAH |
| 408 | 6455 | RTA22200232F.o.06.1.P.Seq | F | M00022151:75 | CH03MAH |
| 409 | 2738 | RTA22200232F.g.16.1.P.Seq | F | M00022050:44 | CH03MAH |
| 410 | 696 | RTA22200232F.m.02.1.P.Seq | F | M00022132:64 | CH03MAH |
| 411 | 379186 | RTA22200012F.c.05.1.P.Seq | F | M00056682:610 | CH16COP |
| 412 | 1588 | RTA22200232F.o.03.1.P.Seq | F | M00022151:411 | CH03MAH |
| 413 | 7007 | RTA22200225F.e.05.1.P.Seq | F | M00005454:33 | CH02COH |
| 414 | 9025 | RTA22200222F.k.23.1.P.Seq | F | M00004080:15 | CH01COH |
| 415 | 650749 | RTA22200007F.i.16.2.P.Seq | F | M00056220:49 | CH15CON |
| 416 | 553158 | RTA22200005F.e.13.1.P.Seq | F | M00055879:511 | CH15CON |
| 417 | 641703 | RTA22200003F.e.18.1.P.Seq | F | M00055628:18 | CH15CON |
| 418 | 833 | RTA22200006F.j.24.2.P.Seq | F | M00056077:512 | CH15CON |
| 419 | 649259 | RTA22200006F.o.01.2.P.Seq | F | M00056108:212 | CH15CON |
| 420 | 451179 | RTA22200011F.i.14.1.P.Seq | F | M00056596:88 | CH16COP |
| 421 | 9505 | RTA22200231F.B.20.1.P.Seq | F | M00007935:15 | CH03MAH |
| 422 | 736728 | RTA22200015F.n.19.1.P.Seq | F | M00057134:31 | CH16COP |
| 423 | 380412 | RTA22200001F.p.23.1.P.Seq | F | M00054918:43 | CH15CON |
| 424 | 642425 | RTA22200011F.k.12.1.P.Seq | F | M00056613:15 | CH16COP |
| 425 | 405073 | RTA22200007F.o.05.1.P.Seq | F | M00056273:11 | CH15CON |
| 426 | 174250 | RTA22200008F.h.09.1.P.Seq | F | M00056475:61 | CH15CON |
| 427 | 72628 | RTA22200017F.d.08.1.P.Seq | F | M00057324:412 | CH16COP |
| 428 | 639029 | RTA22200007F.k.05.2.P.Seq | F | M00056233:63 | CH15CON |
| 429 | 452245 | RTA22200022F.l.05.1.P.Seq | F | M00054963:811 | CH17COHLV |
| 430 | 510254 | RTA22200005F.p.22.1.P.Seq | F | M00056204:14 | CH15CON |
| 431 | 642425 | RTA22200007F.g.19.1.P.Seq | F | M00055912:510 | CH15CON |
| 432 | 51939 | RTA22200005F.i.22.1.P.Seq | F | M00055912:510 | CH15CON |
| 433 | 7379 | RTA22200228F.f.21.1.P.Seq | F | M00006821:21 | CH02COH |
| 434 | 546632 | RTA22200013F.h.16.1.P.Seq | F | M00056858:112 | CH16COP |
| 435 | 734827 | RTA22200011F.m.09.1.P.Seq | F | M00056631:38 | CH16COP |
| 436 | 2554 | RTA22200231F.g.06.1.P.Seq | F | M00007961:65 | CH03MAH |
| 437 | 643285 | RTA22200012F.a.16.1.P.Seq | F | M00056665:111 | CH16COP |
| 438 | 448770 | RTA22200001F.o.07.1.P.Seq | F | M00054793:27 | CH15CON |
| 439 | 375380 | RTA22200023F.e.20.1.P.Seq | F | M00055046:37 | CH17COHLV |
| 440 | 726134 | RTA22200011F.l.10.1.P.Seq | F | M00056620:512 | CH16C0P |
| 441 | 422687 | RTA22200015F.p.03.1.P.Seq | F | M00057143:55 | CH16COP |
| 442 | 448436 | RTA22200012F.n.02.1.P.Seq | F | M00056763:45 | CH16COP |
| 443 | 644893 | RTA22200012F.l.24.1.P.Seq | F | M00056754:15 | CH16COP |
| 444 | 559104 | RTA22200012F.n.21.1.P.Seq | F | M00056771:312 | CH16COP |
| 445 | 551172 | RTA22200016F.g.09.1.P.Seq | F | M00057211:16 | CH16COP |
| 446 | 724296 | RTA22200012F.f.22.1.P.Seq | F | M00056710:67 | CH16COP |
| 447 | 735936 | RTA22200009F.p.13.1.P.Seq | F | M00056346:312 | CH16COP |
| 448 | 556326 | RTA22200023F.k.20.1.P.Seq | F | M00055085:110 | CH17COHLV |
| 449 | 729699 | RTA22200011F.o.04.1.P.Seq | F | M00056646:32 | CH16COP |
| 450 | 550694 | RTA22200022F.a.05.1.P.Seq | F | M00054867:27 | CH17COHLV |
| 451 | 734738 | RTA22200017F.e.11.1.P.Seq | F | M00057337:72 | CH16COP |
| 452 | 404502 | RTA22200007F.p.08.1.P.Seq | F | M00056286:58 | CH15CON |
| 453 | 554151 | RTA22200015F.o.18.1.P.Seq | F | M00057142:17 | CH16COP |
| 454 | 649852 | RTA22200001F.n.24.1.P.Seq | F | M00042914:210 | CH15CON |
| 455 | 734063 | RTA22200011F.m.16.1.P.Seq | F | M0005663:27 | CH16COP |
| 456 | 7279 | RTA22200230F.b.22.1.P.Seq | F | M00007108:41 | CH02COH |
| 457 | 2676 | RTA22200230F.l.05.1.P.Seq | F | M00007204:41 | CH02COH |
| 458 | 649148 | RTA22200006F.k.08.2.P.Seq | F | M00056079:311 | CH15CON |
| 459 | 1953 | RTA22200226F.p.21.1.P.Seq | F | M00006576:24 | CH02COH |
| 460 | 650108 | RTA22200006F.d.04.2.P.Seq | F | M00056018:75 | CH15CON |
| 461 | 515350 | RTA22200005F.g.04.1.P.Seq | F | M00055886:79 | CH15CON |
| 462 | 402494 | RTA22200013F.a.15.1.P.Seq | F | M00056796:85 | CH16COP |
| 463 | 649148 | RTA22200013F.d.09.1.P.Seq | F | M00056821:39 | CH16COP |
| 464 | 833 | RTA22200006F.k.01.2.P.Seq | F | M00056077:512 | CH15CON |
| 465 | 139730 | RTA22200242F.g.02.1.P.Seq | F | M00027014:74 | CH04MAL |
| 466 | 453079 | RTA22200009F.k.11.2.P.Seq | F | M00042835:42 | CH16COP |
| 467 | 546705 | RTA22200003F.p.11.1.P.Seq | F | M00055729:16 | CH15CON |
| 468 | 644903 | RTA22200003F.f.09.1.P.Seq | F | M00055635:74 | CH15CON |
| 469 | 732254 | RTA22200013F.g.01.1.P.Seq | F | M00056842:612 | CH16COP |
| 470 | 561180 | RTA22200003F.e.19.1.P.Seq | F | M00055630:59 | CH15CON |
| 471 | 732254 | RTA22200013F.f.24.1.P.Seq | F | M00056842:612 | CH16COP |
| 472 | 449204 | RTA22200009F.k.07.2.P.Seq | F | M00042834:26 | CH16COP |
| 473 | 185651 | RTA22200242F.f.20.1.P.Seq | F | M00027013:510 | CH04MAL |
| 474 | 639029 | RTA22200013F.l.04.1.P.Seq | F | M00056880:24 | CH16COP |
| 475 | 452986 | RTA22200007F.f.11.1.P.Seq | F | M00056192:54 | CH15CON |
| 476 | 729779 | RTA22200011F.i.05.1.P.Seq | F | M00056594:36 | CH16COP |
| 477 | 646248 | RTA22200005F.o.19.1.P.Seq | F | M00055979:29 | CH15CON |
| 478 | 650448 | RTA22200012F.a.11.1.P.Seq | F | M00056664:27 | CH16COP |
| 479 | 642049 | RTA22200003F.o.23.1.P.Seq | F | M00055726:28 | CH15CON |
| 480 | 728273 | RTA22200012F.f.20.1.P.Seq | F | M00056710:65 | CH16COP |
| 481 | 446139 | RTA22200007F.l.09.2.P.Seq | F | M00056244:37 | CH15CON |
| 482 | 2783 | RTA22200008F.f.20.1.P.Seq | F | M00056456:62 | CH15CON |
| 483 | 642906 | RTA22200012F.n.20.1.P.Seq | F | M00056771:612 | CH16COP |
| 484 | 8332 | RTA22200236F.j.08.1.P.Seq | F | M00022651:24 | CH03MAH |
| 485 | 453470 | RTA22200007F.j.08.2.P.Seq | F | M00056224:58 | CH15CON |
| 486 | 552277 | RTA22200020F.p.05.1.P.Seq | F | M00054706:39 | CH17COHLV |
| 487 | 484029 | RTA22200001F.m.09.1.P.Seq | F | M00042901:13 | CH15CON |
| 488 | 547916 | RTA22200008F.f.11.1.P.Seq | F | M00056448:39 | CH15CON |
| 489 | 649722 | RTA22200006F.m.10.2.P.Seq | F | M00056094:87 | CH15CON |
| 490 | 612572 | RTA22200007F.l.08.2.P.Seq | F | M00056244:28 | CH15CON |
| 491 | 385980 | RTA22200012F.p.05.1.P.Seq | F | M00056784:25 | CH16COP |
| 492 | 141185 | RTA22200007F.i.02.2.P.Seq | F | M00056215:53 | CH15CON |
| 493 | 463824 | RTA22200006F.p.16.2.P.Seq | F | M00056133:49 | CH15CON |
| 494 | 446139 | RTA22200007F.g.14.1.P.Seq | F | M00056201:88 | CH15CON |
| 495 | 725994 | RTA22200011F.h.16.1.P.Seq | F | M00056591.53 | CH16COP |
| 496 | 736679 | RTA22200011F.o.02.1.P.Seq | F | M00056645:66 | CH16COP |
| 497 | 551718 | RTA22200013F.a.18.1.P.Seq | F | M00056799:511 | CH16COP |
| 498 | 640525 | RTA22200003F.m.23.1.P.Seq | F | M00055707:38 | CH15CON |
| 499 | 645210 | RTA22200007F.m.03.1.P.Seq | F | M00056251:16 | CH15CON |
| 500 | 6567 | RTA22200235F.p.09.2.P.Seq | F | M00022569:17 | CH03MAH |
| 501 | 646146 | RTA22200005F.p.10.1.P.Seq | F | M00055985:41 | CH15CON |
| 502 | 4934 | RTA22200227F.a.18.1.P.Seq | F | M00006587:18 | CH02COH |
| 503 | 450791 | RTA22200011F.l.04.1.P.Seq | F | M00056618:66 | CH16COP |
| 504 | 227936 | RTA22200009F.p.09.1.P.Seq | F | M00042879:69 | CH16COP |
| 505 | 9436 | RTA22200020F.m.12.1.P.Seq | F | M00054679:412 | CH17COHLV |
| 506 | 2557 | RTA22200226F.b.14.1.P.Seq | F | M00005610:211 | CH02COH |
| 507 | 11356 | RTA22200228F.g.20.1.P.Seq | F | M00006831:85 | CH02COH |
| 508 | 7571 | RTA22200226F.l.17.1.P.Seq | F | M00005769:13 | CH02COH |
| 509 | 558116 | RTA22200007F.g.09.1.P.Seq | F | M00056199:19 | CH15CON |
| 510 | 216574 | RTA22200007F.o.12.1.P.Seq | F | M00056775:17 | CH16COP |
| 511 | 455145 | RTA22200005F.l.12.1.P.Seq | F | M00055936:57 | CH15CON |
| 512 | 649148 | RTA22200007F.k.01.2.P.Seq | F | M00056231:79 | CH15CON |
| 513 | 648996 | RTA22200007F.m.08.1.P.Seq | F | M00056253:612 | CH15CON |
| 514 | 304253 | RTA22200013F.g.11.1.P.Seq | F | M00056844:110 | CH16COP |
| 515 | 649717 | RTA22200008F.g.10.1.P.Seq | F | M00056466:13 | CH15CON |
| 516 | 5838 | RTA22200226F.h.10.1.P.Seq | F | M00005685:412 | CH02COH |
| 517 | 454050 | RTA22200005F.m.23.1.P.Seq | F | M00055956:52 | CH15CON |
| 518 | 557903 | RTA22200011F.j.17.1.P.Seq | F | M00056608:54 | CH16COP |
| 519 | 1724 | RTA22200240F.k.16.1.P.Seq | F | M00023518:14 | CH04MAL |
| 520 | 734803 | RTA22200011F.n.23.1.P.Seq | F | M00056645:211 | CH16COP |
| 521 | 557948 | RTA22200023F.b.07.1.P.Seq | F | M00055022:84 | CH17COHLV |
| 522 | 5838 | RTA22200229F.i.24.1.P.Seq | F | M00006997:13 | CH02COH |
| 523 | 2334 | RTA22200017F.c.04.1.P.Seq | F | M000573112:511 | CH02COH |
| 524 | 450953 | RTA22200022F.h.12.1.P.Seq | F | M00054937:210 | CH17COHLV |
| 525 | 4840 | RTA22200236F.i.16.1.P.Seq | F | M00022645:15 | CH03MAH |
| 526 | 728421 | RTA22200011F.m.20.1.P.Seq | F | M00056635:111 | CH16COP |
| 527 | 4747 | RTA22200228F.f.23.1.P.Seq | F | M00006822:19 | CH02COH |
| 528 | 648934 | RTA22200005F.m.08.1.P.Seq | F | M00055945:510 | CH15CON |
| 529 | 1787 | RTA22200226F.d.09.1.P.Seq | F | M00005627:210 | CH02COH |
| 530 | 558098 | RTA22200006F.j.17.2.P.Seq | F | M00056074:710 | CH15CON |
| 531 | 1655 | RTA22200222F.k.16.1.P.Seq | F | M00004066:42 | CH01COH |
| 532 | 158601 | RTA22200242F.h.06.1.P.Seq | F | M00027021.811 | CH04MAL |
| 533 | 185486 | RTA22200240F.g.17.1.P.Seq | F | M00023393:512 | CH04MAL |
| 534 | 7110 | RTA22200232F.k.19.1.P.Seq | F | M00022106:44 | CH03MAH |
| 535 | 2543 | RTA22200230F.k.02.1.P.Seq | F | M00007192:56 | CH03MAH |
| 536 | 115762 | RTA22200012F.k.02.1.P.Seq | F | M00056707:45 | CH02COH |
| 537 | 696 | RTA22200243F.p.04.1.P.Seq | F | M00027163:411 | CH04MAL |
| 538 | 1948 | RTA22200232F.l.23.1.P.Seq | F | M00022132:410 | CH03MAH |
| 539 | 696 | RTA22200241F.o.01.1.P.Seq | F | M00026951:711 | CH04MAL |
| 540 | 696 | RTA22200241F.n.24.1.P.Seq | F | M00026951:711 | CH04MAL |
| 541 | 380477 | RTA22200004F.h.14.1.P.Seq | F | M00055791:63 | CH15CON |
| 542 | 638799 | RTA22200002F.b.05.1.P.Seq | F | M00055428:12 | CH15CON |
| 543 | 551982 | RTA22200019F.m.15.1.P.Seq | F | M00054542:18 | CH17COHLV |
| 544 | 551982 | RTA22200021F.k.10.3.P.Seq | F | M00054815:51 | CH17COHLV |
| 545 | 521840 | RTA22200021F.c.20.2.P.Seq | F | M00054739:56 | CH17COHLV |
| 546 | 561180 | RTA22200026F.d.16.1.P.Seq | F | M00055423:110 | CH17COHLV |
| 547 | 556245 | RTA22200020F.i.02.1.P.Seq | F | M00054636:12 | CH17COHLV |
| 548 | 449792 | RTA22200001F.a.07.1.P.Seq | F | M00042345:612 | CH15CON |
| 549 | 549722 | RTA22200020F.d.22.1.P.Seq | F | M00054595:22 | CH17COHLV |
| 550 | 612572 | RTA22200024F.i.24.1.P.Seq | F | M00055216:13 | CH17COHLV |
| 551 | 551235 | RTA22200021F.l.12.3.P.Seq | F | M00054826:25 | CH17COHLV |
| 552 | 449701 | RTA22200009F.h.04.1.P.Seq | F | M00042802:711 | CH16COP |
| 553 | 375380 | RTA22200026F.e.18.1.P.Seq | F | M00055472:83 | CH17COHLV |
| 554 | 56940 | RTA22200021F.l.14.3.P.Seq | F | M00054826:55 | CH17COHLV |
| 555 | 549160 | RTA22200021F.c.19.2.P.Seq | F | M00054739:43 | CH17COHLV |
| 556 | 554151 | RTA22200021F.l.13.3.P.Seq | F | M00054826:310 | CH17COHLV |
| 557 | 727331 | RTS22200015F.b.19.1.P.Seq | F | M00057052:211 | CH16COP |
| 558 | 551502 | RTA22200019F.o.20.1.P.Seq | F | M00054555:712 | CH17COHLV |
| 559 | 612572 | RTA22200024F.j.01.1.P.Seq | F | M00055216:13 | CH17COHLV |
| 560 | 701221 | RTA22200002F.o.23.1.P.Seq | F | M00055556:89 | CH15CON |
| 561 | 378041 | RTA22200004F.b.17.1.P.Seq | F | M00057167:712 | CH15CON |
| 562 | 503491 | RTA22200016F.l.16.1.P.Seq | F | M00055812:51 | CH16COP |
| 563 | 452833 | RTA22200018F.e.01.1.P.Seq | F | M00042520:69 | CH17COHLV |
| 564 | 640974 | RTA22200002F.i.20.1.P.Seq | F | M00055512:68 | CH15CON |
| 565 | 735326 | RTA22200013F.n.08.1.P.Seq | F | M00056898:44 | CH16COP |
| 566 | 555944 | RTA22200021F.i.03.3.P.Seq | F | M00054780:78 | CH17COHLV |
| 567 | 447532 | RTA22200001F.d.07.1.P.Seq | F | M00042556:44 | CH15CON |
| 568 | 455598 | RTA22200002F.k.15.1.P.Seq | F | M00055526:69 | CH15CON |
| 569 | 555734 | RTA22200022F.b.08.1.P.Seq | F | M00054875:34 | CH17COHLV |
| 570 | 446663 | RTA22200001F.a.11.1.P.Seq | F | M00042523:58 | CH15CON |
| 571 | 449862 | RTA22200024F.i.07.1.P.Seq | F | M00055207:14 | CH17COHLV |
| 572 | 549591 | RTA22200023F.o.03.1.P.Seq | F | M00055117:33 | CH17COHLV |
| 573 | 553877 | RTA22200021F.c.23.2.P.Seq | F | M00054740:88 | CH17COHLV |
| 574 | 553501 | RTA22200021F.a.01.2.P.Seq | F | M000504720:412 | CH17COHLV |
| 575 | 1905 | RTA22200020F.p.14.1.P.Seq | F | M00054713:412 | CH17COHLV |
| 576 | 446599 | RTA22200001F.h.20.1.P.Seq | F | M00042720:46 | CH15CON |
| 577 | 559409 | RTA22200024F.d.17.1.P.Seq | F | M00055170:61 | CH17COHLV |
| 578 | 551982 | RTA22200026F.g.14.1.P.Seq | F | M00055485:39 | CH17COHLV |
| 579 | 559057 | RTA22200024F.d.23.1.P.Seq | F | M00055172:44 | CH17COHLV |
| 580 | 446760 | RTA22200001F.b.09.1.P.Seq | F | M00042536:61 | CH15CON |
| 581 | 551502 | RTA22200020F.o.06.1.P.Seq | F | M00054694:74 | CH17COHLV |
| 582 | 446531 | RTA22200025F.o.04.2.P.Seq | F | M00055395:32 | CH17COHLV |
| 583 | 506744 | RTA22200021F.h.07.3.P.Seq | F | M00054772.36 | CH17COHLV |
| 584 | 401849 | RTA22200002F.n.02.1.P.Seq | F | M00055544:22 | CH15CON |
| 585 | 453848 | RTA22200009F.n.04.1.P.Seq | F | M00042853:14 | CH16COP |
| 586 | 456764 | RTA22200004F.c.15.1.P.Seq | F | M00055755:42 | CH15CON |
| 587 | 446371 | RTA22200003F.c.17.1.P.Seq | F | M00055594:11 | CH15CON |
| 588 | 406413 | RTA22200021F.c.09.2.P.Seq | F | M00054734:810 | CH17COHLV |
| 589 | 555103 | RTA22200024F.a.21.1.P.Seq | F | M00055148:411 | CH17COHLV |
| 590 | 735292 | RTA22200015F.c.13.1.P.Seq | F | M00057059:89 | CH16COP |
| 591 | 558534 | RTA22200023F.o.13.1.P.Seq | F | M00055125:61 | CH17COHLV |
| 592 | 727181 | RTA22200016F.k.12.1.P.Seq | F | M00057242:29 | CH16COP |
| 593 | 551117 | RTA22200021F.f.14.3.P.Seq | F | M00054760:112 | CH17COHLV |
| 594 | 464040 | RTA22200024F.g.21.1.P.Seq | F | M00055198:77 | CH17COHLV |
| 595 | 446371 | RTA22200001F.h.03.1.P.Seq | F | M00042712:210 | CH15CON |
| 596 | 728408 | RTA22200014F.p.13.3.P.Seq | F | M00057033:69 | CH16COP |
| 597 | 649259 | RTA22200006F.n.24.2.P.Seq | F | M00056108:212 | CH15CON |
| 598 | 15414 | RTA22200023F.n.18.1.P.Seq | F | M00055113:111 | CH17COHLV |
| 599 | 639240 | RTA22200007F.k.18.2.P.Seq | F | M00056238:57 | CH15CON |
| 600 | 549722 | RTA22200020F.a.09.1.P.Seq | F | M00054568:711 | CH17COHLV |
| 601 | 561499 | RTA22200021F.b.16.2.P.Seq | F | M00054728:210 | CH17COHLV |
| 602 | 639029 | RTA22200002F.k.07.1.P.Seq | F | M00055523:33 | CH15CON |
| 603 | 449512 | RTA22200009F.p.02.1.P.Seq | F | M00042875:54 | CH16COP |
| 604 | 446987 | RTA22200001F.b.20.1.P.Seq | F | M00042540:86 | CH15CON |
| 605 | 466302 | RTA22200004F.m.18.1.P.Seq | F | M00055820:55 | CH15CON |
| 606 | 553802 | RTA22200022F.f.04.1.P.Seq | F | M00054908:67 | CH17COHLV |
| 607 | 639662 | RTA22200004F.h.15.1.P.Seq | F | M0005572:79 | CH15CON |
| 608 | 551527 | RTA22200014F.k.03.2.P.Seq | F | M00056998:58 | CH16COP |
| 609 | 730389 | RTA22200016F.b.02.1.P.Seq | F | M00057162:410 | CH16COP |
| 610 | 640974 | RTA22200002F.n.03.1.P.Seq | F | M00055543:78 | CH15CON |
| 611 | 417155 | RTA22200002F.o.01.1.P.Seq | F | M00055548:512 | CH15CON |
| 612 | 417155 | RTA22200002F.n.24.1.P.Seq | F | M00055548:512 | CH15CON |
| 613 | 451784 | RTA22200008F.d.18.1.P.Seq | F | M00056330:43 | CH15CON |
| 614 | 649152 | RTA22200002F.f.04.1.P.Seq | F | M00055464:65 | CH15CON |
| 615 | 450867 | RTA22200003F.d.19.1.P.Seq | F | M00055613:410 | CH15CON |
| 616 | 143436 | RTA22200003F.d.04.1.P.Seq | F | M00055602:57 | CH15CON |
| 617 | 549395 | RTA22200004F.o.05.1.P.Seq | F | M00055827:55 | CH15CON |
| 618 | 639273 | RTA22200002F.h.02.1.P.Seq | F | M00055496:57 | CH15CON |
| 619 | 506744 | RTA22200021F.m.02.2.P.Seq | F | M00054841:27 | CH17COHLV |
| 620 | 736595 | RTA22200014F.i.22.2.P.Seq | F | M00056990:29 | CH16COP |
| 621 | 230995 | RTA22200016F.i.10.1.P.Seq | F | M00057226:35 | CH16COP |
| 622 | 451784 | RTA22200019F.p.21.1.P.Seq | F | M00054563:39 | CH17COHLV |
| 623 | 226324 | RTA22200020F.n.04.1.P.Seq | F | M00054681:22 | CH17COHLV |
| 624 | 449617 | RTA22200009F.m.12.1.P.Seq | F | M00042849:611 | CH16COP |
| 625 | 451092 | RTA22200026F.h.02.1.P.Seq | F | M00055487:66 | CH17COHLV |
| 626 | 546642 | RTA22200016F.o.16.1.P.Seq | F | M00057275:112 | CH16COP |
| 627 | 553736 | RTA22200022F.j.18.1.P.Seq | F | M00054952:61 | CH17COHLV |
| 628 | 394413 | RTA22200002F.e.15.1.P.Seq | F | M00055456:612 | CH15CON |
| 629 | 556326 | RTA22200021F.j.24.3.P.Seq | F | M00054812:37 | CH17COHLV |
| 630 | 448606 | RTA22200021F.d.08.2.P.Seq | F | M00054741:710 | CH17COHLV |
| 631 | 394413 | RTA22200002F.k.19.1.P.Seq | F | M00055527:52 | CH15CON |
| 632 | 645633 | RTA22200002F.j.09.1.P.Seq | F | M00055516:58 | CH15CON |
| 633 | 551634 | RTA22200021F.j.07.3.P.Seq | F | M00054808:68 | CH17COHLV |
| 634 | 556326 | RTA22200021F.k.01.3.P.Seq | F | M00054812:37 | CH17COHLV |
| 635 | 540787 | RTA22200004F.n.21.1.P.Seq | F | M00055827:42 | CH15CON |
| 636 | 648872 | RTA22200004F.f.05.1.P.Seq | F | M0005575:36 | CH15CON |
| 637 | 643804 | RTA22200004F.f.05.1.P.Seq | F | M00055775:36 | CH15CON |
| 638 | 446139 | RTA22200001F.e.06.1.P.Seq | F | M00042565:18 | CH15CON |
| 639 | 640356 | RTA22200002F.p.15.1.P.Seq | F | M00055563:12 | CH15CON |
| 640 | 379186 | RTA22200015F.i.09.1.P.Seq | F | M00057099:38 | CH16COP |
| 641 | 454927 | RTA22200021F.b.13.2.P.Seq | F | M00054728:58 | CH17COHLV |
| 642 | 401849 | RTA22200004F.h.06.1.P.Seq | F | M00055790:82 | CH15CON |
| 643 | 452414 | RTA22200004F.i.20.1.P.Seq | F | M00055798:36 | CH15CON |
| 644 | 446789 | RTA22200004F.k.24.1.P.Seq | F | M00055807:710 | CH15CON |
| 645 | 189561 | RTA22200022F.n.17.1.P.Seq | F | M00054985:67 | CH17COHLV |
| 646 | 640323 | RTA22200003F.g.22.1.P.Seq | F | M00055653:84 | CH15CON |
| 647 | 558116 | RTA22200024F.h.08.1.P.Seq | F | M00055201:27 | CH17COHLV |
| 648 | 468109 | RTA22200004F.m.20.1.P.Seq | F | M00055820:38 | CH15CON |
| 649 | 481441 | RTA22200003F.b.12.1.P.Seq | F | M00055583:85 | CH15CON |
| 650 | 449956 | RTA22200018F.n.17.1.P.Seq | F | M00043365:36 | CH17COHLV |
| 651 | 727224 | RTA22200013F.p.12.1.P.Seq | F | M00056913:710 | CH16COP |
| 652 | 551907 | RTA22200020F.l.04.1.P.Seq | F | M00054665:88 | CH17COHLV |
| 653 | 447532 | RTA22200004F.a.12.1.P.Seq | F | M00055736:73 | CH15CON |
| 654 | 447532 | RTA22200003F.b.14.1.P.Seq | F | M00055584:311 | CH15CON |
| 655 | 558454 | RTA22200026F.c.17.1.P.Seq | F | M00055417:78 | CH17COHLV |
| 656 | 502683 | RTA22200020F.j.12.1.P.Seq | F | M00054647:82 | CH17COHLV |
| 657 | 446909 | RTA22200023F.n.17.1.P.Seq | F | M00055112:33 | CH17COHLV |
| 658 | 452506 | RTA22200020F.c.23.1.P.Seq | F | M00054587:69 | CH17COHLV |
| 659 | 449792 | RTA22200023F.c.02.1.P.Seq | F | M00055027:68 | CH17COHLV |
| 660 | 549395 | RTA22200020F.i.08.1.P.Seq | F | M00054638:49 | CH17COHLV |
| 661 | 234653 | RTA22200020F.g.14.1.P.Seq | F | M00054621:36 | CH17COHLV |
| 662 | 453911 | RTA22200015F.f.01.1.P.Seq | F | M00057074:39 | CH16COP |
| 663 | 452071 | RTA22200001F.a.18.1.P.Seq | F | M00042529:77 | CH15CON |
| 664 | 451032 | RTA22200018F.g.04.1.P.Seq | F | M00043310:23 | CH17COHLV |
| 665 | 446680 | RTA22200018F.g.17.1.P.Seq | F | M00043313:36 | CH17COHLV |
| 666 | 641884 | RTA22200008F.b.08.1.P.Seq | F | M00056303:24 | CH15CON |
| 667 | 452800 | RTA22200018F.e.16.1.P.Seq | F | M00043301:66 | CH17COHLV |
| 668 | 461835 | RTA22200004F.f.08.1.P.Seq | F | M00055778:69 | CH15CON |
| 669 | 548965 | RTA22200020F.g.08.1.P.Seq | F | M00054617:19 | CH17COHLV |
| 670 | 734793 | RTA22200016F.c.20.1.P.Seq | F | M00057174:812 | CH16COP |
| 671 | 539955 | RTA22200008F.a.12.1.P.Seq | F | M00056295:46 | CH15CON |
| 672 | 561892 | RTA22200020F.f.11.1.P.Seq | F | M00054609:61 | CH17COHLV |
| 673 | 562292 | RTA22200020F.e.06.1.P.Seq | F | M00054596:711 | CH17COHLV |
| 674 | 420686 | RTA22200001F.j.02.1.P.Seq | F | M00042735:72 | CH15CON |
| 675 | 9436 | RTA22200004F.f.20.1.P.Seq | F | M00055781:35 | CH15CON |
| 676 | 1013 | RTA22200004F.e.21.1.P.Seq | F | M00055774:51 | CH15CON |
| 677 | 412364 | RTA22200008F.b.07.1.P.Seq | F | M00056303:33 | CH15CON |
| 678 | 44424 | RTA22200018F.j.01.1.P.Seq | F | M00043327:82 | CH17COHLV |
| 679 | 394413 | RTA22200001F.j.18.1.P.Seq | F | M00042742:45 | CH15CON |
| 680 | 449617 | RTA22200008F.c.08.1.P.Seq | F | M00056312:14 | CH15CON |
| 681 | 455032 | RTA22200018F.l.07.1.P.Seq | F | M00043347:712 | CH17COHLV |
| 682 | 185400 | RTA22200025F.b.17.1.P.Seq | F | M00055279:512 | CH17COHLV |
| 683 | 453911 | RTA22200015F.e.24.1.P.Seq | F | M00057074:39 | CH16COP |
| 684 | 650297 | RTA22200004F.e.23.1.P.Seq | F | M00055774:73 | CH15COP |
| 685 | 185400 | RTA22200018F.k.02.1.P.Seq | F | M00043338:23 | CH17COHLV |
| 686 | 449512 | RTA22200025F.c.06.1.P.Seq | F | M00055283:82 | CH17COHLV |
| 687 | 44424 | RTA22200018F.i.24.1.P.Seq | F | M00043327:82 | CH17COHLV |
| 688 | 556216 | RTA22200025F.h.09.1.P.Seq | F | M00055350:61 | CH17COHLV |
| 689 | 448677 | RTA22200025F.f.07.1.P.Seq | F | M00055324:810 | CH17COHLV |
| 690 | 375380 | RTA22200025F.e.16.1.P.Seq | F | M00055319:11 | CH17COHLV |
| 691 | 379341 | RTA22200018F.a.24.1.P.Seq | F | M00042442:12 | CH17COHLV |
| 692 | 376988 | RTA22200025F.h.03.1.P.Seq | F | M00055346:42 | CH17COHLV |
| 693 | 559806 | RTA22200025F.g.20.1.P.Seq | F | M00055344:89 | CH17COHLV |
| 694 | 550195 | RTA22200025F.j.07.1.P.Seq | F | M00055368:310 | CH17COHLV |
| 695 | 562221 | RTA22200025F.k.09.1.P.Seq | F | M00055374:18 | CH17COHLV |
| 696 | 211 | RTA22200248F.o.19.1.P.Seq | F | M00027607:85 | CH04MAL |
| 697 | 6751 | RTA22200248F.o.01.1.P.Seq | F | M00027604:710 | CH04MAL |
| 698 | 6751 | RTA22200248F.n.24.1.P.Seq | F | M00027604:710 | CH04MAL |
| 699 | 11619 | RTA22200250F.d.17.1.P.Seq | F | M00027806:85 | CH04MAL |
| 700 | 2883 | RTA22200249F.l.14.1.P.Seq | F | M00027717:75 | CH04MAL |
| 701 | 9784 | RTA22200249F.c.12.1.P.Seq | F | M00027628:11 | CH04MAL |
| 702 | 649722 | RTA22200010F.i.15.1.P.Seq | F | M00056424:612 | CH16COP |
| 703 | 2888 | RTA22200224F.e.01.1.P.Seq | F | M00004869:46 | CH02COH |
| 704 | 10340 | RTA22200233F.p.01.1.P.Seq | F | M00021681:32 | CH03MAH |
| 705 | 1649 | RTA22200234F.g.21.1.P.Seq | F | M00022246:88 | CH03MAH |
| 706 | 4325 | RTA22200234F.i.13.1.P.Seq | F | M00022259:27 | CH03MAH |
| 707 | 10882 | RTA22200249F.f.21.1.P.Seq | F | M00027658:73 | CH04MAL |
| 708 | 10342 | RTA22200233F.l.20.1.P.Seq | F | M00021628:47 | CH03MAH |
| 709 | 6474 | RTA22200224F.e.16.1.P.Seq | F | M00004972:51 | CH02COH |
| 710 | 10340 | RTA22200233F.o.24.1.P.Seq | F | M00021681:32 | CH03MAH |
| 711 | 734723 | RTA22200010F.k.06.1.P.Seq | F | M00056480:312 | CH16COP |
| 712 | 452142 | RTA22200010F.i.16.1.P.Seq | F | M00056424:86 | CH16COP |
| 713 | 185432 | RTA22200249F.o.24.1.P.Seq | F | M00027742:21 | CH04MAL |
| 714 | 11456 | RTA22200224F.m.02.1.P.Seq | F | M00005385:110 | CH02COH |
| 715 | 508892 | RTA22200010F.p.07.1.P.Seq | F | M0056517:73 | CH16COP |
| 716 | 67 | RTA22200224F.f.14.1.P.Seq | F | M00005019:42 | CH02COH |
| 717 | 2636 | RTA22200224F.m.17.1.P.Seq | F | M00005389:31 | CH02COH |
| 718 | 735028 | RTA22200011F.e.08.1.P.Seq | F | M00056556:71 | CH16COP |
| 719 | 1924 | RTA22200224F.n.16.1.P.Seq | F | M00005395:49 | CH02COH |
| 720 | 640116 | RTA22200010F.m.24.1.P.Seq | F | M00056503:711 | CH16COP |
| 721 | 6546 | RTA22200225F.b.13.1.P.Seq | F | M00005420:31 | CH02COH |
| 722 | 730866 | RTA22200010F.p.03.1.P.Seq | F | M00056515:35 | CH16COP |
| 723 | 4829 | RTA22200224F.c.02.1.P.Seq | F | M00004850:75 | CH02COH |
| 724 | 546632 | RTA22200010F.o.12.1.P.Seq | F | M00056512:36 | CH16COP |
| 725 | 549934 | RTA22200019F.i.19.1.P.Seq | F | M00054510:89 | CH17COHLV |
| 726 | 649655 | RTA22200010F.o.22.1.P.Seq | F | M00056514:58 | CH16COP |
| 727 | 62016 | RTA22200019F.k.05.1.P.Seq | F | M00054520:25 | CH17COHLV |
| 728 | 2783 | RTA22200019F.k.18.1.P.Seq | F | M00054524:22 | CH17COHLV |
| 729 | 3876 | RTA22200019F.e.03.1.P.Seq | F | M00043504:76 | CH17COHLV |
| 730 | 20036 | RTA22200019F.f.11.1.P.Seq | F | M00054486:211 | CH17COHLV |
| 731 | 644032 | RTA22200013F.e.06.1.P.Seq | F | M00056824:51 | CH16COP |
| 732 | 451636 | RTA22200018F.o.24.1.P.Seq | F | M00043374:22 | CH17COHLV |
| 733 | 3428 | RTA22200237F.e.01.1.P.Seq | F | M00022716:36 | CH03MAH |
| 734 | 643954 | RTA22200012F.p.22.1.P.Seq | F | M00056789:510 | CH16COP |
| 735 | 456506 | RTA22200015F.p.23.1.P.Seq | F | M00057150:310 | CH16COP |
| 736 | 449269 | RTA22200014F.p.17.1.P.Seq | F | M00057035:39 | CH16COP |
| 737 | 732712 | RTA22200014F.p.03.1.P.Seq | F | M00057029:16 | CH16COP |
| 738 | 696 | RTA22200236F.c.13.1.P.Seq | F | M00022598:512 | CH03MAH |
| 739 | 456528 | RTA22200014F.n.13.1.P.Seq | F | M00057019:82 | CH16COP |
| 740 | 4043 | RTA22200228F.k.21.2.P.Seq | F | M00006871:61 | CH02COH |
| 741 | 3639 | RTA22200235F.g.13.1.P.Seq | F | M00022472:51 | CH03MAH |
| 742 | 1024 | RTA22200244F.p.09.1.P.Seq | F | M00027229:56 | CH04MAL |
| 743 | 1247 | RTA22200236F.i.17.1.P.Seq | F | M00022645:37 | CH03MAH |
| 744 | 4934 | RTA22200228F.i.13.2.P.Seq | F | M00006846:43 | CH02COH |
| 745 | 901 | RTA22200237F.f.07.1.P.Seq | F | M00022724:44 | CH03MAH |
| 746 | 452726 | RTA22200014F.i.04.1.P.Seq | F | M00057003:29 | CH16COP |
| 747 | 725825 | RTA22200014F.i.23.1.P.Seq | F | M00056990:311 | CH16COP |
| 748 | 456808 | RTA22200014F.a.19.1.P.Seq | F | M00056924:26 | CH16COP |
| 749 | 729295 | RTA22200014F.a.21.1.P.Seq | F | M00056925:37 | CH16COP |
| 750 | 551907 | RTA22200005F.a.18.1.P.Seq | F | M00055850:63 | CH15CON |
| 751 | 551527 | RTA22200014F.k.03.1.P.Seq | F | M00056998:58 | CH16COP |
| 752 | 7098 | RTA22200237F.n.22.1.P.Seq | F | M00022831:87 | CH03MAH |
| 753 | 4589 | RTA22200225F.k.24.1.P.Seq | F | M00005501:55 | CH02COH |
| 754 | 554812 | RTA22200014F.g.21.1.P.Seq | F | M00056969:38 | CH16COP |
| 755 | 3114 | RTA22200232F.g.01.1.P.Seq | F | M00022015:611 | CH03MAH |
| 756 | 6031 | RTA22200227F.m.13.1.P.Seq | F | M00006731:43 | CH02COH |
| 757 | 185628 | RTA22200242F.j.21.1.P.Seq | F | M00027035:89 | CH04MAL |
| 758 | 24719 | RTA22200244F.j.04.1.P.Seq | F | M00027197:67 | CH04MAL |
| 759 | 3428 | RTA22200237F.d.24.1.P.Seq | F | M00022716:36 | CH03MAH |
| 760 | 2676 | RTA22200227F.j.24.1.P.Seq | F | M00006695:88 | CH02COH |
| 761 | 649148 | RTA22200007F.j.24.1.P.Seq | F | M00056231:79 | CH15CON |
| 762 | 234605 | RTA22200009F.c.17.2.P.Seq | F | M00042515:110 | CH16COP |
| 763 | 2224 | RTA22200232F.g.14.1.P.Seq | F | M00022049:27 | CH03MAH |
| 764 | 185642 | RTA22200243F.a.07.1.P.Seq | F | M00027076:67 | CH04MAL |
| 765 | 649655 | RTA22200007F.d.16.1.P.Seq | F | M00056171:811 | CH15CON |
| 766 | 2854 | RTA22200231F.o.23.1.P.Seq | F | M00007998:67 | CH03MAH |
| 767 | 453470 | RTA22200007F.j.08.1.P.Seq | F | M00056224:58 | CH15CON |
| 768 | 11012 | RTA22200227F.k.14.1.P.Seq | F | M00006704:76 | CH02COH |
| 769 | 535208 | RTA22200005F.c.23.1.P.Seq | F | M00055869:16 | CH15CON |
| 770 | 448606 | RTA22200014F.j.14.1.P.Seq | F | M00056994:33 | CH16COP |
| 771 | 12304 | RTA22200244F.b.17.1.P.Seq | F | M00027172:33 | CH04MAL |
| 772 | 2756 | RTA22200229F.h.17.1.P.Seq | F | M00006989:54 | CH02COH |
| 773 | 367 | RTA22200005F.d.04.1.P.Seq | F | M00055871:65 | CH15CON |
| 774 | 11351 | RTA22200229F.c.09.1.P.Seq | F | M00006937:63 | CH02COH |
| 775 | 6858 | RTA22200228F.n.03.2.P Seq | F | M00006888:75 | CH02COH |
| 776 | 7750 | RTA22200241F.g.01.1.P.Seq | F | M00026899:711 | CH04MAL |
| 777 | 6923 | RTA22200232F.h.12.1.P.Seq | F | M00022060:69 | CH03MAH |
| 778 | 11552 | RTA22200232F.p.03.1.P.Seq | F | M00022163:38 | CH03MAH |
| 779 | 12448 | RTA22200243F.c.10.1.P.Seq | F | M00027090:58 | CH04MAL |
| 780 | 2944 | RTA22200227F.m.11.1.P.Seq | F | M00006731:22 | CH02COH |
| 781 | 10342 | RTA22200231F.l.03.1.P.Seq | F | M00007981:24 | CH03MAH |
| 782 | 9026 | RTA22200222F.o.20.1.P.Seq | F | M00004304:47 | CH01COH |
| 783 | 10342 | RTA22200238F.m.23.1.P.Seq | F | M00022997:38 | CH03MAH |
| 784 | 6455 | RTA22200235F.f.08.1.P.Seq | F | M00022465:65 | CH03MAH |
| 785 | 6455 | RTA22200232F.b.15.1.P.Seq | F | M00021886:66 | CH03MAH |
| 786 | 3416 | RTA22200227F.k.16.1.P.Seq | F | M00006705:79 | CH02COH |
| 787 | 3416 | RTA22200229F.a.12.1.P.Seq | F | M00006923:85 | CH02COH |
| 788 | 2889 | RTA22200228F.n.19.2.P.Seq | F | M00006894:13 | CH02COH |
| 789 | 7393 | RTA22200241F.l.09.1.P.Seq | F | M00026934:59 | CH04MAL |
| 790 | 14390 | RTA22200244F.i.10.1.P.Seq | F | M00027193:35 | CH04MAL |
| 791 | 661 | RTA22200241F.a.15.1.P.Seq | F | M00026856:311 | CH04MAL |
| 792 | 452992 | RTA22200013F.e.07.1.P.Seq | F | M00056826:212 | CH16COP |
| 793 | 1943 | RTA22200235F.k.21.2.P.Seq | F | M00022528:812 | CH03MAH |
| 794 | 2027 | RTA22200244F.l.20.1.P.Seq | F | M00027207:59 | CH04MAL |
| 795 | 5482 | RTA22200225F.p.15.1.P.Seq | F | M00005565:65 | CH02COH |
| 796 | 650493 | RTA22200007F.c.10.1.P.Seq | F | M00056156:112 | CH15CON |
| 797 | 640318 | RTA22200007F.b.04.1.P.Seq | F | M00056145:24 | CH15CON |
| 798 | 646309 | RTA22200011F.p.23.1.P.Seq | F | M00056661:55 | CH16COP |
| 799 | 4316 | RTA22200248F.e.15.1.P.Seq | F | M00027543:77 | CH04MAL |
| 800 | 449701 | RTA22200012F.d.12.1.P.Seq | F | M00056695:89 | CH16COP |
| 801 | 560367 | RTA22200013F.e.03.1.P.Seq | F | M00056824:310 | CH16COP |
| 802 | 9997 | RTA22200238F.o.18.1.P.Seq | F | M00023015:42 | CH03MAH |
| 803 | 649106 | RTA22200012F.f.03.1.P.Seq | F | M00056707:52 | CH16COP |
| 804 | 461835 | RTA22200007F.b.18.1.P.Seq | F | M00056150:312 | CH15CON |
| 805 | 640590 | RTA22200003F.m.09.1.P.Seq | F | M00055703:35 | CH15CON |
| 806 | 648340 | RTA22200003F.m.09.1.P.Seq | F | M00056139:54 | CH15CON |
| 807 | 554812 | RTA22200014F.g.21.2.P.Seq | F | M00056969:38 | CH16COP |
| 808 | 447035 | RTA22200007F.m.06.1.P.Seq | F | M00056252:511 | CH15CON |
| 809 | 1208 | RTA22200238F.g.03.1.P.Seq | F | M00022908:83 | CH03MAH |
| 810 | 3114 | RTA22200232F.f.24.1.P.Seq | F | M00022015:611 | CH03MAH |
| 811 | 3114 | RTA22200232F.n.22.1.P.Seq | F | M00022149:36 | CH03MAH |
| 812 | 734078 | RTA22200013F.j.12.1.P.Seq | F | M00056870:510 | CH16COP |
| 813 | 450323 | RTA22200015F.o.17.1.P.Seq | F | M00057141:42 | CH16COP |
| 814 | 11567 | RTA22200232F.f.16.1.P.Seq | F | M00022010:81 | CH03MAH |
| 815 | 11567 | RTA22200232F.c.14.1.P.Seq | F | M00021915:510 | CH03MAH |
| 816 | 6660 | RTA22200222F.j.01.1.P.Seq | F | M00003986:712 | CH01COH |
| 817 | 9026 | RTA22200222F.n.12.1.P.Seq | F | M00004198:111 | CH01COH |
| 818 | 185539 | RTA22200243F.b.09.1.P.Seq | F | M00027084:810 | CH04MAL |
| 819 | 3224 | RTA22200222F.d.01.1.P.Seq | F | M00001537:610 | CH01COH |
| 820 | 95700 | RTA22200240F.l.12.1.P.Seq | F | M00026804:43 | CH04MAL |
| 821 | 4439 | RTA22200222F.n.01.1.P.Seq | F | M00004167:411 | CH01COH |
| 822 | 3428 | RTA22200236F.n.08.1.P.Seq | F | M00022667:511 | CH03MAH |
| 823 | 1456 | RTA22200236F.p.05.1.P.Seq | F | M00022681:56 | CH03MAH |
| 824 | 11343 | RTA22200228F.g.03.1.P.Seq | F | M00006822:59 | CH02COH |
| 825 | 729206 | RTA22200012F.k.20.1.P.Seq | F | M00056746:46 | CH16COP |
| 826 | 558371 | RTA22200015F.p.13.1.P.Seq | F | M00057147:11 | CH16COP |
| 827 | 451589 | RTA22200011F.m.21.1.P.Seq | F | M00056635:59 | CH16COP |
| 828 | 404475 | RTA22200012F.f.10.1.P.Seq | F | M00056708:36 | CH16COP |
| 829 | 734582 | RTA22200017F.e.07.1.P.Seq | F | M00057334:61 | CH16COP |
| 830 | 729779 | RTA22200011F.i.11.1.P.Seq | F | M00056596:52 | CH16COP |
| 831 | 555244 | RTA22200023F.l.23.1.P.Seq | F | M00055093:85 | CH17COHLV |
| 832 | 449269 | RTA22200014F.p.17.2.P.Seq | F | M00057035:39 | CH16COP |
| 833 | 4609 | RTA22200227F.a.11.1.P.Seq | F | M00006583:83 | CH02COH |
| 834 | 640318 | RTA22200011F.i.13.1.P.Seq | F | M00056596:56 | CH16COP |
| 835 | 729851 | RTA22200011F.j.22.1.P.Seq | F | M00056610:812 | CH16COP |
| 836 | 11028 | RTA22200230F.a.15.1.P.Seq | F | M00007098:35 | CH02COH |
| 837 | 643924 | RTA22200008F.e.17.1.P.Seq | F | M00056345:53 | CH15CON |
| 838 | 630259 | RTA22200005F.i.07.1.P.Seq | F | M00055908:811 | CH15CON |
| 839 | 11286 | RTA22200230F.k.07.1.P.Seq | F | M00007194:56 | CH02COH |
| 840 | 185651 | RTA22200248F.g.16.1.P.Seq | F | M00027556:710 | CH04MAL |
| 841 | 7379 | RTA22200230F.d.19.1.P.Seq | F | M00007128:76 | CH02COH |
| 842 | 728408 | RTA22200014F.p.13.2.P.Seq | F | M00057033:69 | CH16COP |
| 843 | 646309 | RTA22200007F.e.15.1.P.Seq | F | M00056184:83 | CH15CON |
| 844 | 405073 | RTA22200003F.m.14.1.P.Seq | F | M00055704:47 | CH15CON |
| 845 | 185489 | RTA22200241F.j.24.1.P.Seq | F | M00026926:35 | CH04MAL |
| 846 | 447326 | RTA22200005F.i.15.1.P.Seq | F | M00055909:58 | CH15CON |
| 847 | 111006 | RTA22200227F.c.15.1.P.Seq | F | M00006610:48 | CH02COH |
| 848 | 6863 | RTA22200227F.a.23.1.P.Seq | F | M00006588:86 | CH02COH |
| 849 | 11351 | RTA22200225F.p.12.1.P.Seq | F | M00005563:212 | CH02COH |
| 850 | 401553 | RTA2220000SF.f.13.1.P.Seq | F | M00055884:21 | CH15CON |
| 851 | 504513 | RTA22200007F.h.19.1.P.Seq | F | M00056213:14 | CH15CON |
| 852 | 645979 | RTA22200012F.g.11.1.P.Seq | F | M00056713:78 | CH16COP |
| 853 | 6923 | RTA22200233F.b.21.1.P.Seq | F | M00008023:412 | CH03MAH |
| 854 | 1924 | RTA22200229F.l.18.1.P.Seq | F | M00007034:26 | CH02COH |
| 855 | 5838 | RTA22200230F.f.02.1.P.Seq | F | M00007136:35 | CH02COH |
| 856 | 2062 | RTA22200230F.f.24.1.P.Seq | F | M00007146:611 | CH02COH |
| 857 | 447388 | RTA22200003F.b.06.1.P.Seq | F | M00055581:21 | CH15CON |
| 858 | 12419 | RTA22200241F.c.04.1.P.Seq | F | M00026865:711 | CH04MAL |
| 859 | 3224 | RTA22200222F.c.24.1.P.Seq | F | M00001537:610 | CH01COH |
| 860 | 5474 | RTA22200230F.i.23.1.P.Seq | F | M00007174:46 | CH02COH |
| 861 | 3522 | RTA22200227F.f.23.1.P.Seq | F | M00006650:45 | CH02COH |
| 862 | 731785 | RTA22200012F.o.21.1.P.Seq | F | M00056777:67 | CH16COP |
| 863 | 3765 | RTA22200228F.d.10.1.P.Seq | F | M00006803:37 | CH02COH |
| 864 | 640323 | RTA22200012F.a.10.1.P.Seq | F | M00056664:76 | CH16COP |
| 865 | 379105 | RTA22200012F.i.11.1.P.Seq | F | M00056728:85 | CH16COP |
| 866 | 448029 | RTA22200012F.n.03.1.P.Seq | F | M00056764:58 | CH16COP |
| 867 | 650476 | RTA22200005F.h.07.1.P.Seq | F | M00055893:35 | CH15CON |
| 868 | 640525 | RTA22200007F.f.12.1.P.Seq | F | M00056192:82 | CH15CON |
| 869 | 390124 | RTA22200007F.n.09.1.P.Seq | F | M00056267:52 | CH15CON |
| 870 | 464029 | RTA22200023F.p.21.1.P.Seq | F | M00055137:34 | CH17COHLV |
| 871 | 468109 | RTA22200005F.e.18.1.P.Seq | F | M00055880:612 | CH15CON |
| 872 | 21669 | RTA22200023F.h.20.1.P.Seq | F | M00055064:512 | CH17COHLV |
| 873 | 651088 | RTA22200007F.j.07.2.P.Seq | F | M00056224:210 | CH15CON |
| 874 | 2737 | RTA22200023F.d.15.1.P.Seq | F | M00055039:71 | CH17COHLV |
| 875 | 556421 | RTA22200005F.e.14.1.P.Seq | F | M00055879:44 | CH15CON |
| 876 | 452245 | RTA22200023F.c.23.1.P.Seq | F | M00055034:81 | CH17COHLV |
| 877 | 447539 | RTA22200005F.e.22.1.P.Seq | F | M00055882:19 | CH15CON |
| 878 | 546642 | RTA22200014F.o.20.2.P.Seq | F | M00057029:312 | CH16COP |
| 879 | 236368 | RTA22200006F.j.23.2.P.Seq | F | M00056077:56 | CH15CON |
| 880 | 644523 | RTA22200012F.b.12.1.P.Seq | F | M00056673:56 | CH16COP |
| 881 | 729173 | RTA22200012F.k.02.1.P.Seq | F | M00056739:411 | CH16COP |
| 882 | 8315 | RTA22200231F.a.06.1.P.Seq | F | M00007927:31 | CH03MAH |
| 883 | 450463 | RTA22200007F.i.11.2.P.Seq | F | M00056218:73 | CH15C0N |
| 884 | 650856 | RTA22200012F.o.13.1.P.Seq | F | M00056775:31 | CH16COP |
| 885 | 648109 | RTA22200005F.i.14.1.P.Seq | F | M00055909:71 | CH15CON |
| 886 | 726644 | RTA22200013F.d.19.1.P.Seq | F | M00056822:711 | CH16COP |
| 887 | 727224 | RTA22200013F.h.19.1.P.Seq | F | M00056859:412 | CH16COP |
| 888 | 557906 | RTA22200023F.h.18.1.P.Seq | F | M00055063:71 | CH17COHLV |
| 889 | 502683 | RTA22200007F.e.22.1.P.Seq | F | M00056185:46 | CH15CON |
| 890 | 728408 | RTA22200011F.i.06.1.P.Seq | F | M00056594:310 | CH16COP |
| 891 | 647952 | RTA22200005F.j.11.1.P.Seq | F | M00055919:16 | CH15CON |
| 892 | 639991 | RTA22200009F.l.23.2.P.Seq | F | M00042846:49 | CH16COP |
| 893 | 735346 | RTA22200016F.m.21.1.P.Seq | F | M00057266:44 | CH16COP |
| 894 | 102655 | RTA22200007F.i.23.2.P.Seq | F | M00056222:62 | CH15CON |
| 895 | 553629 | RTA22200012F.o.03.1.P.Seq | F | M00056772:58 | CH16COP |
| 896 | 1609 | RTA22200226F.i.23.1.P.Seq | F | M00005708:27 | CH02COH |
| 897 | 641884 | RTA22200005F.l.16.1.P.Seq | F | M00055937:32 | CH15CON |
| 898 | 648872 | RTA22200007F.p.10.1.P.Seq | F | M00056286:112 | CH15CON |
| 899 | 644242 | RTA22200005F.i.20.1.P.Seq | F | M00055911:56 | CH15CON |
| 900 | 63559 | RTA22200244F.f.14.1.P.Seq | F | M00027183:21 | CH04MAL |
| 901 | 550108 | RTA22200012F.o.24.1.P.Seq | F | M00056779:810 | CH16COP |
| 902 | 374306 | RTA22200011F.n.05.1.P.Seq | F | M00056639:52 | CH16COP |
| 903 | 5838 | RTA22200226F.c.19.1.P.Seq | F | M00005621:88 | CH02COH |
| 904 | 645530 | RTA22200006F.k.07.2.P.Seq | F | M00056079:67 | CH15CON |
| 905 | 649732 | RTA22200012F.b.05.1.P.Seq | F | M00056669:77 | CH16COP |
| 906 | 649143 | RTA22200007F.p.01.1.P.Seq | F | M00056283:52 | CH15CON |
| 907 | 7571 | RTA22200225F.h.24.1.P.Seq | F | M00005481:36 | CH02COH |
| 908 | 4572 | RTA22200236F.j.09.1.P.Seq | F | M00022651:31 | CH03MAH |
| 909 | 2147 | RTA22200229F.j.14.1.P.Seq | F | M00007006:11 | CH02COH |
| 910 | 462659 | RTA22200008F.h.10.1.P.Seq | F | M00056475:62 | CH15CON |
| 911 | 727723 | RTA22200012F.m.21.1.P.Seq | F | M00056761:65 | CH16COP |
| 912 | 2636 | RTA22200226F.g.21.1.P.Seq | F | M00005657:112 | CH02COH |
| 913 | 500959 | RTA22200005F.k.21.1.P.Seq | F | M00055931:51 | CH15CON |
| 914 | 3428 | RTA22200231F.f.07.1.P.Seq | F | M00007954:24 | CH03MAH |
| 915 | 734929 | RTA22200011F.h.24.1.P.Seq | F | M00056592:47 | CH16COP |
| 916 | 453592 | RTA22200001F.g.19.1.P.Seq | F | M00042706:14 | CH15CON |
| 917 | 15414 | RTA22200026F.d.21.1.P.Seq | F | M00055423:810 | CH17COHLV |
| 918 | 648959 | RTA22200002F.m.22.1.P.Seq | F | M00055542:19 | CH15CON |
| 919 | 453470 | RTA22200007F.g.06.1.P.Seq | F | M00056198:47 | CH15CON |
| 920 | 649272 | RTA22200007F.k.03.2.P.Seq | F | M00056232:56 | CH15CON |
| 921 | 1699 | RTA22200012F.p.06.1.P.Seq | F | M00056781:512 | CH16COP |
| 922 | 649719 | RTA22200006F.m.21.2.P.Seq | F | M00056098:81 | CH15CON |
| 923 | 562805 | RTA22200023F.d.08.1.P.Seq | F | M00055037:51 | CH17COHLV |
| 924 | 452204 | RTA22200001F.p.24.1.P.Seq | F | M00054918:311 | CH15CON |
| 925 | 549178 | RTA22200023F.d.22.1.P.Seq | F | M00055041:52 | CH17COHLV |
| 926 | 639177 | RTA22200015F.n.24.1.P.Seq | F | M00057134:710 | CH16COP |
| 927 | 562550 | RTA22200022F.i.05.1.P.Seq | F | M00054941:74 | CH17COHLVI |
| 928 | 561807 | RTA22200004F.l.07.1.P.Seq | F | M00055808:711 | CH15CON |
| 929 | 641373 | RTA22200006F.m.23.2.P.Seq | F | M00056099:79 | CH15CON |
| 930 | 514418 | RTA22200022F.a.15.1.P.Seq | F | M00054870:49 | CH17COHLV |
| 931 | 567078 | RTA22200006F.b.18.2.P.Seq | F | M00056007:111 | CH15CON |
| 932 | 643061 | RTA22200006F.n.01.2.P.Seq | F | M00056099:811 | CH15CON |
| 933 | 549160 | RTA22200020F.i.09.1.P.Seq | F | M00054638:38 | CH17COHLV |
| 934 | 449269 | RTA22200009F.e.07.1.P.Seq | F | M00042770:212 | CH16COP |
| 935 | 453082 | RTA22200001F.e.03.1.P.Seq | F | M00042564:811 | CH15CON |
| 936 | 418135 | RTA22200026F.a.09.1.P.Seq | F | M00055406:45 | CH17COHLV |
| 937 | 2783 | RTA22200023F.b.23.1.P.Seq | F | M00055027:47 | CH17COHLV |
| 938 | 549435 | RTA22200007F.j.06.2.P.Seq | F | M00056223:73 | CH15CON |
| 939 | 446614 | RTA22200009F.i.13.2.P.Seq | F | M00042816:64 | CH16COP |
| 940 | 449477 | RTA22200012F.p.04.1.P.Seq | F | M00056780:32 | CH16COP |
| 941 | 454380 | RTA22200013F.c.09.1.P.Seq | F | M00056815:21 | CH16COP |
| 942 | 450914 | RTA22200013F.c.05.1.P.Seq | F | M00056812:58 | CH16COP |
| 943 | 736860 | RTA22200016F.p.14.1.P.Seq | F | M00057283:56 | CH16COP |
| 944 | 727224 | RTA22200011F.h.19.1.P.Seq | F | M00056592:64 | CH16COP |
| 945 | 644242 | RTA22200006F.a.19.2.P.Seq | F | M00056000:612 | CH15CON |
| 946 | 562550 | RTA22200022F.p.09.1.P.Seq | F | M00055002:54 | CH17COHLV |
| 947 | 649148 | RTA22200007F.j.24.2.P.Seq | F | M00056231:79 | CH15CON |
| 948 | 375889 | RTA22200023F.c.13.1.P.Seq | F | M00055032:16 | CH17COHLV |
| 949 | 449437 | RTA22200009F.e.03.1.P.Seq | F | M00042767:42 | CH16COP |
| 950 | 449044 | RTA22200006F.b.07.2.P.Seq | F | M00056003:56 | CH15CON |
| 951 | 555318 | RTA22200022F.e.11.1.P.Seq | F | M00054903:312 | CH17COHLV |
| 952 | 456764 | RTA22200005F.k.20.1.P.Seq | F | M00055931:31 | CH15CON |
| 953 | 11567 | RTA22200231F.g.22.1.P.Seq | F | M00007965:710 | CH03MAH |
| 954 | 3522 | RTA22200229F.j.18.1.P.Seq | F | M00007007:85 | CH02COH |
| 955 | 456528 | RTA22200016F.p.05.1.P.Seq | F | M00057280:16 | CH16COP |
| 956 | 639142 | RTA22200013F.m.02.1.P.Seq | F | M00056887:68 | CH16COP |
| 957 | 446371 | RTA22200001F.e.10.1.P.Seq | F | M00042567:31 | CH15CON |
| 958 | 554742 | RTA22200026F.a.03.1.P.Seq | F | M00055405:85 | CH17COHLV |
| 959 | 448029 | RTA22200009F.m.10.1.P.Seq | F | M00042848:712 | CH16COP |
| 960 | 551380 | RTA22200022F.j.23.1.P.Seq | F | M00054952:86 | CH17COHLV |
| 961 | 551527 | RTA22200020F.n.22.1.P.Seq | F | M00654689:512 | CH17COHLV |
| 962 | 729295 | RTA22200014F.a.21.2.P.Seq | F | M00056925:37 | CH16COP |
| 963 | 349744 | RTA22200022F.m.17.1.P.Seq | F | M00054975:34 | CH17COHLV |
| 964 | 648996 | RTA22200004F.d.22.1.P.Seq | F | M00055768:25 | CH15CON |
| 965 | 447126 | RTA22200001F.e.18.1.P.Seq | F | M00042573:17 | CH15CON |
| 966 | 730866 | RTA22200013F.p.20.1.P.Seq | F | M00056916:22 | CH16COP |
| 967 | 420686 | RTA22200020F.o.02.1.P.Seq | F | M00054692:41 | CH17COHLV |
| 968 | 451753 | RTA22200017F.b.07.1.P.Seq | F | M00057304:51 | CH16COP |
| 969 | 451380 | RTA22200021F.d.12.2.P.Seq | F | M00054743:52 | CH17COHLV |
| 970 | 645530 | RTA22200003F.l.11.1.P.Seq | F | M00055689:67 | CH15CON |
| 971 | 554703 | RTA22200016F.m.22.1.P.Seq | F | M00057266:712 | CH16COP |
| 972 | 562835 | RTA22200015F.n.20.1.P.Seq | F | M00057134:11 | CH16COP |
| 973 | 732764 | RTA22200016F.m.14.1.P.Seq | F | M00057259:28 | CH16COP |
| 974 | 556216 | RTA22200019F.l.17.1.P.Seq | F | M00054534:42 | CH17COHLV |
| 975 | 728779 | RTA22200017F.c.19.1.P.Seq | F | M00057318:29 | CH16COP |
| 976 | 414739 | RTA22200002F.n.19.1.P.Seq | F | M00055547:410 | CH15CON |
| 977 | 551514 | RTA22200022F.k.05.1.P.Seq | F | M00054953:710 | CH17COHLV |
| 978 | 550107 | RTA22200023F.p.02.1.P.Seq | F | M00055130:71 | CH17COHLV |
| 979 | 726786 | RTA22200017F.a.10.1.P.Seq | F | M00057291:26 | CH16COP |
| 980 | 456747 | RTA22200001F.f.23.1.P.Seq | F | M00042700:11 | CH15CON |
| 981 | 562550 | RTA22200023F.n.13.1.P.Seq | F | M00055111:43 | CH17COHLV |
| 982 | 549722 | RTA22200002F.p.13.1.P.Seq | F | M00055560:66 | CH15CON |
| 983 | 640525 | RTA22200004F.i.10.1.P.Seq | F | M00055796:510 | CH15CON |
| 984 | 455542 | RTA22200009F.f.23.1.P.Seq | F | M00042787:59 | CH16COP |
| 985 | 9436 | RTA22200016F.n.12.1.P.Seq | F | M00057270:54 | CH16COP |
| 986 | 380284 | RTA22200004F.c.17.1.P.Seq | F | M00055755:83 | CH15CON |
| 987 | 556260 | RTA22200020F.p.08.1.P.Seq | F | M00054707:55 | CH17COHLV |
| 988 | 650476 | RTA22200013F.p.21.1.P.Seq | F | M00056916:64 | CH16COP |
| 989 | 554500 | RTA22200006F.n.14.2.P.Seq | F | M00056103:812 | CH15CON |
| 990 | 422375 | RTA22200016F.n.16.1.P.Seq | F | M00057272:23 | CH16COP |
| 991 | 456528 | RTA22200019F.l.23.1.P.Seq | F | M00054535:89 | CH17COHLV |
| 992 | 644190 | RTA22200009F.m.23.1.P.Seq | F | M00042853:73 | CH16COP |
| 993 | 554080 | RTA22200014F.o.19.3.P.Seq | F | M00057028:49 | CH16COP |
| 994 | 546705 | RTA22200021F.i.06.3.P.Seq | F | M00054781:24 | CH17COHLV |
| 995 | 558337 | RTA22200021F.a.02.2.P.Seq | F | M00054720:611 | CH17COHLV |
| 996 | 449269 | RTA22200014F.p.17.3.P.Seq | F | M00057035:39 | CH16COP |
| 997 | 645799 | RTA22200014F.d.22.2.P.Seq | F | M00056952:84 | CH16COP |
| 998 | 456506 | RTA22200001F.l.10.1.P.Seq | F | M00042889:19 | CH15CON |
| 999 | 218416 | RTA22200026F.c.09.1.P.Seq | F | M00055414:111 | CH17COHLV |
| 1000 | 455820 | RTA22200023F.m.12.1.P.Seq | F | M00055097:76 | CH17COHLV |
| 1001 | 554703 | RTA22200024F.p.19.1.P.Seq | F | M00055262:211 | CH17COHLV |
| 1002 | 650204 | RTA22200008F.c.16.1.P.Seq | F | M00056313:67 | CH15CON |
| 1003 | 456808 | RTA22200014F.a.19.2.P.Seq | F | M00056924:26 | CH16COP |
| 1004 | 420686 | RTA22200004F.n.01.1.P.Seq | F | M00055821:16 | CH15CON |
| 1005 | 378373 | RTA22200023F.n.20.1.P.Seq | F | M00055114:111 | CH17COHLV |
| 1006 | 463824 | RTA22200004F.a.22.1.P.Seq | F | M00055740:69 | CH15CON |
| 1007 | 24939 | RTA22200016F.i.15.1.P.Seq | F | M00057229:66 | CH16COP |
| 1008 | 556561 | RTA22200022F.n.08.1.P.Seq | F | M00054981:36 | CH17COHLV |
| 1009 | 380406 | RTA22200001F.l.13.1.P.Seq | F | M00042890:38 | CH15CON |
| 1010 | 456764 | RTA22200004F.a.10.1.P.Seq | F | M00055736:46 | CH15CON |
| 1011 | 725703 | RTA22200016F.a.04.1.P.Seq | F | M00057154:46 | CH16COP |
| 1012 | 185465 | RTA22200009F.g.10.1.P.Seq | F | M00042793:76 | CH16COP |
| 1013 | 5830 | RTA22200013F.m.20.1.P.Seq | F | M00056894:76 | CH16COP |
| 1014 | 539955 | RTA22200013F.o.22.1.P.Seq | F | M00056909:49 | CH16COP |
| 1015 | 640747 | RTA22200002F.o.17.1.P.Seq | F | M00055553:37 | CH16COP |
| 1016 | 500630 | RTA22200013F.n.12.1.P.Seq | F | M00056901:16 | CH16COP |
| 1017 | 448511 | RTA22200022F.n.14.1.P.Seq | F | M00054984:37 | CH17COHLV |
| 1018 | 405073 | RTA22200002F.b.21.1.P.Seq | F | M00055438:36 | CH15CON |
| 1019 | 641439 | RTA22200003F.l.08.1.P.Seq | F | M00055688:24 | CH15CON |
| 1020 | 406092 | RTA22200004F.a.20.1.P.Seq | F | M00055740:212 | CH15CON |
| 1021 | 559806 | RTA22200022F.o.02.1.P.Seq | F | M00054987:32 | CH17COHLV |
| 1022 | 380284 | RTA22200002F.l.11.1.P.Seq | F | M00055529:22 | CH15COP |
| 1023 | 560700 | RTA22200026F.f.08.1.P.Seq | F | M00055475:78 | CH17COHLV |
| 1024 | 552879 | RTA22200024F.m.13.1.P.Seq | F | M00055242:11 | CH17COHLV |
| 1025 | 640590 | RTA22200003F.l.14.1.P.Seq | F | M00055692:57 | CH15CON |
| 1026 | 641683 | RTA22200003F.k.24.1.P.Seq | F | M00055685:51 | CH15CON |
| 1027 | 648934 | RTA22200003F.a.23.1.P.Seq | F | M00055578:89 | CH15CON |
| 1028 | 557948 | RTA22200021F.g.09.3.P.Seq | F | M00054766:24 | CH17COHLV |
| 1029 | 377094 | RTA22200021F.d.05.2.P.Seq | F | M00054741:510 | CH17COHLV |
| 1030 | 449617 | RTA22200026F.c.15.1.P.Seq | F | M00055415:811 | CH17COHLV |
| 1031 | 978 | RTA22200004F.m.17.1.P.Seq | F | M00055820:58 | CH15CON |
| 1032 | 607430 | RTA22200024F.n.10.1.P.Seq | F | M00055247:111 | CH17COHLV |
| 1033 | 641837 | RTA22200003F.b.20.1.P.Seq | F | M00055586:65 | CH15CON |
| 1034 | 449750 | RTA22200021F.h.08.3.P.Seq | F | M00054773:112 | CH17COHLV |
| 1035 | 646780 | RTA22200004F.m.23.1.P.Seq | F | M00055820:710 | CH15CON |
| 1036 | 546642 | RTA22200014F.o.20.3.P.Seq | F | M00057029:312 | CH16COP |
| 1037 | 642906 | RTA22200014F.i.13.2.P.Seq | F | M00056985:35 | CH16COP |
| 1038 | 552879 | RTA22200024F.g.10.1.P.Seq | F | M00055196:19 | CH17COHLV |
| 1039 | 644205 | RTA22200003F.d.03.1.P.Seq | F | M00055602:710 | CH15CON |
| 1040 | 506744 | RTA22200021F.n.01.2.P.Seq | F | M00054841:27 | CH17COHLV |
| 1041 | 557797 | RTA22200024F.c.03.1.P.Seq | F | M00055154:64 | CH17COHLV |
| 1042 | 640356 | RTA22200004F.h.18.1.P.Seq | F | M00055792:57 | CH15CON |
| 1043 | 462659 | RTA22200001F.i.16.1.P.Seq | F | M00042732:79 | CH15CON |
| 1044 | 645633 | RTA22200008F.a.01.1.P.Seq | F | M00056291:25 | CH15CON |
| 1045 | 237288 | RTA22200002F.g.19.1.P.Seq | F | M00055495:63 | CH15CON |
| 1046 | 454343 | RTA22200016F.d.13.1.P.Seq | F | M00057181:46 | CH16COP |
| 1047 | 386543 | RTA22200024F.n.20.1.P.Seq | F | M00055198:67 | CH17COHLV |
| 1048 | 446404 | RTA22200003F.b.10.1.P.Seq | F | M00055582:54 | CH15CON |
| 1049 | 456528 | RTA22200014F.n.13.2.P.Seq | F | M00057019:82 | CH16COP |
| 1050 | 456528 | RTA22200015F.l.08.1.P.Seq | F | M00057118:32 | CH16COP |
| 1051 | 452781 | RTA22200004F.o.03.1.P.Seq | F | M00055827:36 | CH15CON |
| 1052 | 551671 | RTA22200021F.b.20.1.PSeq | F | M00054729:51 | CH17COHLV |
| 1053 | 644242 | RTA22200004F.h.08.1.P.Seq | F | M00055791:45 | CH15CON |
| 1054 | 561892 | RTA22200025F.n.14.2.P.Seq | F | M00055391:76 | CH17COHLV |
| 1055 | 450429 | RTA22200004F.e.09.1.P.Seq | F | M00055771:41 | CH15CON |
| 1056 | 533588 | RTA22200001F.k.13.1.P.Seq | F | M00042881:38 | CH15CON |
| 1057 | 553877 | RTA22200004F.f.03.1.P.Seq | F | M00055775210 | CH15CON |
| 1058 | 650195 | RTA22200004F.f.23.1.P.Seq | F | M00055782:62 | CH15CON |
| 1059 | 934861 | RTA22200020F.b.22.1.P.Seq | F | M00054580:411 | CH17COHLV |
| 1060 | 650195 | RTA22200001F.k.22.1.P.Seq | F | M00042886:61 | CH15CON |
| 1061 | 562835 | RTA22200025F.m.22.2.P.Seq | F | M00055388:78 | CH17COHLV |
| 1062 | 736816 | RTA22200015F.b.20.1.P.Seq | F | M00057052:79 | CH16COP |
| 1063 | 403632 | RTA22200020F.b.08.1.P.Seq | F | M00054575:31 | CH17COHLV |
| 1064 | 390124 | RTA22200021F.n.17.2.P.Seq | F | M00054849:811 | CH17COHLV |
| 1065 | 390124 | RTA22200016F.i.16.1.P.Seq | F | M00057230:412 | CH16COP |
| 1066 | 422687 | RTA22200015F.e.14.1.P.Seq | F | M00057072:52 | CH16COP |
| 1067 | 394413 | RTA22200001F.k.11.1.P.Seq | F | M00042750:29 | CH15CON |
| 1068 | 549178 | RTA22200020F.a.18.1.P.Seq | F | M00054571:31 | CH17COHLV |
| 1069 | 453079 | RTA22200004F.e.Al.1.P.Seq | F | M00055771:111 | CH15CON |
| 1070 | 463824 | RTA22200001F.j.03.1.P.Seq | F | M00042735:17 | CH15CON |
| 1071 | 736595 | RTA22200016F.d.16.1.P.Seq | F | M0057182:211 | CH16COP |
| 1072 | 102655 | RTA22200004F.m.02.1.P.Seq | F | M00055816:61 | CH15CON |
| 1073 | 448606 | RTA22200014F.j.14.2.P.Seq | F | M00056994:33 | CH16COP |
| 1074 | 504513 | RTA22200004F.n.12.1.P.Seq | F | M00055823:411 | CH15CON |
| 1075 | 20036 | RTA22200004F.f.12.1.P.Seq | F | M00055779:12 | CH15CON |
| 1076 | 530883 | RTA22200016F.d.08.1.P.Seq | F | M00057180:811 | CH16COP |
| 1077 | 447126 | RTA22200004F.o.18.1.P.Seq | F | M00055832:512 | CH15CON |
| 1078 | 556561 | RTA22200025F.d.13.1.P.Seq | F | M00055302:62 | CH17COHLV |
| 1079 | 455096 | RTA22200020F.e.15.1.P.Seq | F | M00054600:77 | CH17COHLV |
| 1080 | 549320 | RTA22200020F.g.16.1.P.Seq | F | M00054621:411 | CH17COHLV |
| 1081 | 560984 | RTA22200025F.d.20.1.P.Seq | F | M00055306:79 | CH17COHLV |
| 1082 | 450791 | RTA22200018F.k.03.1.P.Seq | F | M00043338:13 | CH17COHLV |
| 1083 | 16556 | RTA22200025F.c.02.1.P.Seq | F | M00055281:58 | CH17COHLV |
| 1084 | 402707 | RTA22200018F.d.23.1.P.Seq | F | M00042520:64 | CH17COHLV |
| 1085 | 557903 | RTA22200025F.b.15.1.P.Seq | F | M00055279:78 | CH17COHLV |
| 1086 | 451243 | RTA22200018F.i.18.1.P.Seq | F | M00043324:44 | CH17COHLV |
| 1087 | 452506 | RTA22200025F.b.06.1.P.Seq | F | M00055274:62 | CH17COHLV |
| 1088 | 554703 | RTA22200025F.g.23.1.P.Seq | F | M00055345:811 | CH17COHLV |
| 1089 | 449580 | RTA22200018F.c.03.1.P.Seq | F | M00042450:810 | CH17COHLV |
| 1090 | 3316 | RTA22200025F.i.02.1.P.Seq | F | M00055356:36 | CH17COHLV |
| 1091 | 97507 | RTA22200025F.i.22.1.P.Seq | F | M00055364:51 | CH17COHLV |
| 1092 | 556216 | RTA22200025F.k.07.1.P.Seq | F | M00055373:410 | CH17COHLV |
| 1093 | 185401 | RTA22200250F.d.22.1.P.Seq | F | M00027808:710 | CH04MAL |
| 1094 | 3758 | RTA22200248F.o.08.1.P.Seq | F | M00027605:55 | CH04MAL |
| 1095 | 95700 | RTA22200248F.l.15.1.P.Seq | F | M00027588:16 | CH04MAL |
| 1096 | 2478 | RTA22200250F.d.12.1.P.Seq | F | M00027803:810 | CH04MAL |
| 1097 | 550267 | RTA22200011F.d.14.1.P.Seq | F | M00056552:210 | CH16COP |
| 1098 | 185652 | RTA22200250F.c.19.1.P.Seq | F | M00027786:21 | CH04MAL |
| 1099 | 55798 | RTA22200250F.a.04.1.P.Seq | F | M00027757:26 | CH04MAL |
| 1100 | 5078 | RTA22200011F.a.19.1.P.Seq | F | M00056529:89 | CH16COP |
| 1101 | 9784 | RTA22200249F.h.22.1.P.Seq | F | M00027681:42 | CH04MAL |
| 1102 | 2245 | RTA22200234F.e.08.1.P.Seq | F | M00022216:46 | CH03MAH |
| 1103 | 11606 | RTA22200234F.e.19.1.P.Seq | F | M00022221:46 | CH03MAH |
| 1104 | 2245 | RTA22200234F.e.21.1.P.Seq | F | M00022221:46 | CH03MAH |
| 1105 | 551172 | RTA22200011F.b.19.1.P.Seq | F | M00056537:19 | CH16COP |
| 1106 | 729175 | RTA22200010F.i.01.1.P.Seq | F | M00056420:47 | CH16COP |
| 1107 | 6317 | RTA22200224F.e.13.1.P.Seq | F | M00004971:74 | CH02COH |
| 1108 | 2478 | RTA22200234F.f.21.1.P.Seq | F | M00022235:311 | CH03MAH |
| 1109 | 4727 | RTA22200234F.f.10.1.P.Seq | F | M00022231:512 | CH03MAH |
| 1110 | 185598 | RTA22200249F.p.13.1.P.Seq | F | M00027747:41 | CH04MAL |
| 1111 | 736349 | RTA22200010F.j.16.1.P.Seq | F | M00056434:571 | CH16COP |
| 1112 | 8001 | RTA22200224F.g.15.1.P.Seq | F | M00005313:22 | CH02COH |
| 1113 | 189561 | RTA22200010F.i.20.1.P.Seq | F | M00056425:88 | CH16COP |
| 1114 | 728131 | RTA22200010F.n.22.1.P.Seq | F | M00056507:210 | CH16COP |
| 1115 | 560984 | RTA22200019F.k.07.1.P.Seq | F | M00054521:64 | CH17COHLV |
| 1116 | 549945 | RTA22200019F.j.03.1.P.Seq | F | M00054513:112 | CH17COHLV |
| 1117 | 554785 | RTA22200019F.g.21.1.P.Seq | F | M00054499:511 | CH17COHLV |
| 1118 | 554785 | RTA22200019F.h.01.1.P.Seq | F | M00054499:511 | CH17COHLV |
| 1119 | 551235 | RTA22200019F.e.14.1.P.Seq | F | M00043506:89 | CH17COHLV |
| 1120 | 2634 | RTA22200237F.e.18.1.P.Seq | F | M00022720:39 | CH03MAH |
| 1121 | 548858 | RTA22200019F.g.06.1.P.Seq | F | M00054493:110 | CH17COHLV |
| 1122 | 15625 | RTA22200005F.n.15.1.P.Seq | F | M00055966:46 | CH15CON |
| 1123 | 649259 | RTA22200006F.a.21.2.P.Seq | F | M00056000:49 | CH15CON |
| 1124 | 55267 | RTA22200005F.b.15.1.P.Seq | F | M00055856:67 | CH15CON |
| 1125 | 7436 | RTA22200237F.b.08.1.P.Seq | F | M00022697:412 | CH03MAH |
| 1126 | 451794 | RTA22200005F.c.13.1.P.Seq | F | M00055866:12 | CH15CON |
| 1127 | 5744 | RTA22200227F.l.15.1.P.Seq | F | M00006719:512 | CH02COH |
| 1128 | 3516 | RTA22200228F.n.13.2.P.Seq | F | M00006892:69 | CH02COH |
| 1129 | 730555 | RTA22200014F.j.23.1.P.Seq | F | M00056997:89 | CH16COP |
| 1130 | 3085 | RTA22200237F.n.05.1.P.Seq | F | M00022829:86 | CH03MAH |
| 1131 | 638854 | RTA22200005F.b.09.1.P.Seq | F | M00055854:54 | CH15CON |
| 1132 | 7379 | RTA22200225F.l.06.1.P.Seq | F | M00005501:79 | CH02COH |
| 1133 | 185562 | RTA22200248F.i.11.1.P.Seq | F | M00027571:311 | CH04MAL |
| 1134 | 452491 | RTA22200005F.a.16.1.P.Seq | F | M00055849:48 | CH15CON |
| 1135 | 646248 | RTA22200005F.a.14.1.P.Seq | F | M00055848:86 | CH15CON |
| 1136 | 6056 | RTA22200235F.f.14.1.P.Seq | F | M00022468:512 | CH03MAH |
| 1137 | 643103 | RTA22200005F.d.12.1.P.Seq | F | M00055873:211 | CH15CON |
| 1138 | 6923 | RTA22200237F.h.10.1.P.Seq | F | M00022741:211 | CH03MAH |
| 1139 | 6923 | RTA22200237F.h.16.1.P.Seq | F | M00022745:37 | CH03MAH |
| 1140 | 901 | RTA22200237F.o.03.1.P.Seq | F | M00022831:34 | CH03MAH |
| 1141 | 901 | RTA22200237F.n.23.1.P.Seq | F | M00022831:19 | CH03MAH |
| 1142 | 367 | RTA22200236F.h.18.1.P.Seq | F | M00022641:310 | CH03MAH |
| 1143 | 4043 | RTA22200228F.l.13.2.P.Seq | F | M00006873:21 | CH02COH |
| 1144 | 3299 | RTA22200236F.h.19.1.P.Seq | F | M00022641:56 | CH03MAH |
| 1145 | 11881 | RTA22200238F.e.15.1.P.Seq | F | M00022899:39 | CH03MAH |
| 1146 | 9113 | RTA22200230F.l.04.1.P.Seq | F | M00007204:712 | CH02COH |
| 1147 | 185460 | RTA22200243F.p.24.1.P.Seq | F | M00027165:611 | CH04MAL |
| 1148 | 185716 | RTA22200241F.d.03.1.P.Seq | F | M00026873:511 | CH04MAL |
| 1149 | 5753 | RTA22200227F.o.22.1.P.Seq | F | M00006756:68 | CH02COH |
| 1150 | 24939 | RTA22200012F.e.06.1.P.Seq | F | M00056701:58 | CH16COP |
| 1151 | 649684 | RTA22200007F.a.14.1.P.Seq | F | M00056140:87 | CH15CON |
| 1152 | 642109 | RTA22200012F.h.03.1.P.Seq | F | M00056717:34 | CH16COP |
| 1153 | 15035 | RTA22200007F.l.11.1.P.Seq | F | M00056246:23 | CH15CON |
| 1154 | 649354 | RTA22200007F.a.15.1.P.Seq | F | M00056140:57 | CH15CON |
| 1155 | 4465 | RTA22200228F.e.19.1.P.Seq | F | M00006811:412 | CH02COH |
| 1156 | 647952 | RTA22200007F.b.02.1.P.Seq | F | M00056144:39 | CH15CON |
| 1157 | 455601 | RTA22200005F.p.18.1.P.Seq | F | M00055990:25 | CH15CON |
| 1158 | 641901 | RTA22200005F.p.07.1.P.Seq | F | M00055984:32 | CH15CON |
| 1159 | 446878 | RTA22200009F.b.21.2.P.Seq | F | M00042466:86 | CH16COP |
| 1160 | 7436 | RTA22200232F.h.08.1.P.Seq | F | M00022058:11 | CH03MAH |
| 1161 | 2245 | RTA22200230F.i.03.1.P.Seq | F | M00007166:56 | CH02COH |
| 1162 | 3531 | RTA22200227F.o.01.1.P.Seq | F | M00006746:26 | CH02COH |
| 1163 | 9625 | RTA22200240F.k.21.1.P.Seq | F | M00023520:77 | CH04MAL |
| 1164 | 727489 | RTA22200012F.l.19.1.P.Seq | F | M00056754:14 | CH16COP |
| 1165 | 159925 | RTA22200240F.j.14.1.P.Seq | F | M00023428:43 | CH04MAL |
| 1166 | 645210 | RTA22200012F.g.17.1.P.Seq | F | M00056715:54 | CH16COP |
| 1167 | 157629 | RTA22200235F.d.24.1.P.Seq | F | M00022453:84 | CH03MAH |
| 1168 | 8375 | RTA22200231F.l.07.1.P.Seq | F | M00007982:611 | CH03MAH |
| 1169 | 4319 | RTA22200230F.e.02.1.P.Seq | F | M00007129:68 | CH02COH |
| 1170 | 4045 | RTA22200231F.n.07.1.P.Seq | F | M00007992:78 | CH03MAH |
| 1171 | 185642 | RTA22200240F.p.21.1.P.Seq | F | M00026848:711 | CH04MAL |
| 1172 | 7436 | RTA22200238F.l.11.1.P.Seq | F | M00022974:410 | CH03MAH |
| 1173 | 3531 | RTA22200227F.n.24.1.P.Seq | F | M00006746:26 | CH02COH |
| 1174 | 644776 | RTA22200012F.g.21.1.P.Seq | F | M00056715:58 | CH02COH |
| 1175 | 8354 | RTA22200238F.d.13.1.P.Seq | F | M00022892:77 | CH03MAH |
| 1176 | 2099 | RTA22200243F.f.05.1.P.Seq | F | M00027111:84 | CH04MAL |
| 1177 | 449956 | RTA22200011F.l.18.1.P.Seq | F | M00056624:85 | CH16COP |
| 1178 | 649106 | RTA22200003F.o.09.1.P.Seq | F | M00055723:55 | CH15CON |
| 1179 | 452414 | RTA22200005F.i.09.1.P.Seq | F | M00055908:512 | CH15CON |
| 1180 | 732712 | RTA22200011F.k.21.1.P.Seq | F | M00056616:110 | CH16COP |
| 1181 | 185562 | RTA22200225F.m.01.1.P.Seq | F | M00026938:64 | CH04MAL |
| 1182 | 3516 | RTA22200241F.i.17.1.P.Seq | F | M00005485:19 | CH02COH |
| 1183 | 185562 | RTA22200241F.l.24.1.P.Seq | F | M00026938:64 | CH04MAL |
| 1184 | 185460 | RTA22200241F.a.08.1.P.Seq | F | M00026854:57 | CH04MAL |
| 1185 | 10947 | RTA22200237F.l.16.1.P.Seq | F | M00022813:18 | CH03MAH |
| 1186 | 452856 | RTA22200007F.d.13.1.P.Seq | F | M00056169:66 | CH15CON |
| 1187 | 558767 | RTA22200015F.m.17.1.P.Seq | F | M00057127:77 | CH16COP |
| 1188 | 15035 | RTA22200008F.e.22.1.P.Seq | F | M00056438:16 | CH15CON |
| 1189 | 556421 | RTA22200005F.h.23.1.P.Seq | F | M00055906:612 | CH15CON |
| 1190 | 7082 | RTA22200235F.o.24.2.P.Seq | F | M00022565:15 | CH03MAH |
| 1191 | 452523 | RTA22200013F.j.07.1.P.Seq | F | M00056868:59 | CH16COP |
| 1192 | 3242 | RTA22200230F.a.01.1.P.Seq | F | M00007092:63 | CH02COH |
| 1193 | 6660 | RTA22200222F.i.24.1.P.Seq | F | M00003986:712 | CH01COH |
| 1194 | 547 | RTA22200244F.j.02.1.P.Seq | F | M00027197:77 | CH04MAL |
| 1195 | 121213 | RTA22200011F.i.23.1.P.Seq | F | M00056599:411 | CH16COP |
| 1196 | 4378 | RTA22200226F.p.15.1.P.Seq | F | M00005830:410 | CH02COH |
| 1197 | 185554 | RTA22200244F.m.17.1.P.Seq | F | M00027217:73 | CH04MAL |
| 1198 | 185482 | RTA22200241F.d.04.1.P.Seq | F | M00026873:28 | CH04MAL |
| 1199 | 185715 | RTA22200240F.l.14.1.P.Seq | F | M00026805:24 | CH04MAL |
| 1200 | 66017 | RTA22200243F.d.10.1.P.Seq | F | M00027097:711 | CH04MAL |
| 1201 | 403111 | RTA22200007F.j.14.1.P.Seq | F | M00056226:612 | CH15CON |
| 1202 | 3224 | RTA22200222F.o.18.1.P.Seq | F | M00004296:711 | CH01COH |
| 1203 | 966 | RTA22200238F.k.11.1.P.Seq | F | M00022961:211 | CH03MAH |
| 1204 | 3639 | RTA22200235F.j.05.2.P.Seq | F | M00022509:26 | CH03MAH |
| 1205 | 5388 | RTA22200243F.k.17.1.P.Seq | F | M00027139:36 | CH04MAL |
| 1206 | 3299 | RTA22200238F.b.05.1.P.Seq | F | M00022872:25 | CH03MAH |
| 1207 | 23760 | RTA22200241F.n.13.1.P.Seq | F | M00026949:810 | CH04MAL |
| 1208 | 729384 | RTA22200012F.n.06.1.P.Seq | F | M00056765:512 | CH16COP |
| 1209 | 46559 | RTA22200016F.g.16.1.P.Seq | F | M00057215:22 | CH16COP |
| 1210 | 449750 | RTA22200022F.n.05.1.P.Seq | F | M00054980:32 | CH17COHLV |
| 1211 | 735936 | RTA22200011F.i.02.1.P.Seq | F | M00056593:55 | CH16COP |
| 1212 | 607430 | RTA22200005F.o.04.1.P.Seq | F | M00055971:57 | CH15CON |
| 1213 | 452856 | RTA22200007F.g.23.1.P.Seq | F | M00056205:41 | CH15CON |
| 1214 | 557903 | RTA22200016F.g.07.1.P.Seq | F | M00057211:67 | CH16COP |
| 1215 | 453112 | RTA22200001F.g.01.1.P.Seq | F | M00042700:43 | CH15CON |
| 1216 | 645900 | RTA22200006F.i.24.2.P.Seq | F | M00056067:48 | CH15CON |
| 1217 | 415114 | RTA22200002F.m.20.1.P.Seq | F | M00055542:31 | CH15CON |
| 1218 | 418763 | RTA22200004F.l.11.1.P.Seq | F | M00055811:18 | CH15CON |
| 1219 | 2245 | RTA22200230F.a.12.1.P.Seq | F | M00007097:24 | CH02COH |
| 1220 | 403668 | RTA22200012F.o.18.1.P.Seq | F | M00056777:33 | CH16COP |
| 1221 | 15427 | RTA22200020F.m.08.1.P.Seq | F | M00054677:42 | CH17COHLV |
| 1222 | 555714 | RTA22200020F.n.23.1.P.Seq | F | M00054691:55 | CH17COHLV |
| 1223 | 555830 | RTA22200022F.d.19.1.P.Seq | F | M00054899:67 | CH17COHLV |
| 1224 | 4620 | RTA22200231F.e.13.1.P.Seq | F | M00007951:15 | CH03MAH |
| 1225 | 171511 | RTA22200012F.p.21.1.P.Seq | F | M00056789:34 | CH16COP |
| 1226 | 451401 | RTA22200008F.h.08.1.P.Seq | F | M00056475:312 | CH15CON |
| 1227 | 447501 | RTA22200002F.e.01.1.P.Seq | F | M00055453:51 | CH15CON |
| 1228 | 460445 | RTA22200022F.p.12.1.P.Seq | F | M00055005:28 | CH17COHLV |
| 1229 | 375814 | RTA22200004F.k.20.1.P.Seq | F | M00055806:59 | CH15CON |
| 1230 | 449356 | RTA22200026F.d.19.1.P.Seq | F | M00055423:78 | CH17COHLV |
| 1231 | 468736 | RTA22200022F.i.10.1.P.Seq | F | M00054943:33 | CH17COHLV |
| 1232 | 548858 | RTA22200023F.a.04.1.P.Seq | F | M00055011:54 | CH17COHLV |
| 1233 | 3693 | RTA22200240F.g.16.1.P.Seq | F | M00023393:32 | CH04MAL |
| 1234 | 642973 | RTA22200005F.k.19.1.P.Seq | F | M00055931:13 | CH15CON |
| 1235 | 561180 | RTA22200012F.n.04.1.P.Seq | F | M00056765:110 | CH16COP |
| 1236 | 453708 | RTA22200020F.p.07.1.P.Seq | F | M00054707:28 | CH17COHLV |
| 1237 | 645305 | RTA22200001F.p.17.1.P.Seq | F | M00054917:69 | CH15CON |
| 1238 | 463487 | RTA22200007F.e.12.1.P.Seq | F | M00056184:48 | CH15CON |
| 1239 | 11131 | RTA22200230F.b.14.1.P.Seq | F | M00007105:312 | CH02COH |
| 1240 | 561807 | RTA22200020F.k.18.1.P.Seq | F | M00054660:65 | CH17COHLV |
| 1241 | 452800 | RTA22200023F.n.03.1.P.Seq | F | M00055104:212 | CH17COHLV |
| 1242 | 372960 | RTA22200012F.p.07.1.P.Seq | F | M00056782:26 | CH16COP |
| 1243 | 449317 | RTA22200009F.k.10.2.P.Seq | F | M00042835:31 | CH16COP |
| 1244 | 730759 | RTA22200011F.n.19.1.P.Seq | F | M00056643:76 | CH16COP |
| 1245 | 9113 | RTA22200226F.i.12.1.P.Seq | F | M00005704:410 | CH02COH |
| 1246 | 630259 | RTA22200004F.k.14.1.P.Seq | F | M00055805:410 | CH15CON |
| 1247 | 3516 | RTA22200226F.o.20.1.P.Seq | F | M00005819:211 | CH02COH |
| 1248 | 447494 | RTA22200004F.j.14.1.P.Seq | F | M00055802:72 | CH15CON |
| 1249 | 554500 | RTA22200019F.n.10.1.P.Seq | F | M00054547:59 | CH17COHLV |
| 1250 | 639662 | RTA22200001F.p.19.1.P.Seq | F | M00054917:412 | CH15CON |
| 1251 | 421 | RTA22200009F.o.23.1.P.Seq | F | M00042869:56 | CH16COP |
| 1252 | 736014 | RTA22200016F.o.19.1.P.Seq | F | M00057277:39 | CH16COP |
| 1253 | 643061 | RTA22200006F.m.24.2.P.Seq | F | M00056099:811 | CH15CON |
| 1254 | 9113 | RTA22200229F.m.02.1.P.Seq | F | M00007035:56 | CH02COH |
| 1255 | 650856 | RTA22200007F.i.19.2.P.Seq | F | M00056221:55 | CH15CON |
| 1256 | 476223 | RTA22200009F.g.09.1.P.Seq | F | M00042792:64 | CH16COP |
| 1257 | 737088 | RTA22200011F.f.01.1.P.Seq | F | M00056564:59 | CH16COP |
| 1258 | 449512 | RTA22200022F.P.03.1.P.Seq | F | M00055000:64 | CH17COHLV |
| 1259 | 449457 | RTA22200023F.m.21.1.P.Seq | F | M00055100:48 | CH17COHLV |
| 1260 | 521901 | RTA22200022F.l.07.1.P.Seq | F | M00054964:811 | CH17COHLV |
| 1261 | 175799 | RTA22200001F.f.12.1.P.Seq | F | M00042695:85 | CH15CON |
| 1262 | 550108 | RTA22200012F.p.01.1.P.Seq | F | M00056779:810 | CH15CON |
| 1263 | 203605 | RTA22200003F.e.03.1.P.Seq | F | M00055618:16 | CH16COP |
| 1264 | 450429 | RTA22200013F.b.10.1.P.Seq | F | M00056805:29 | CH16COP |
| 1265 | 2478 | RTA22200238F.j.18.1.P.Seq | F | M00022956:29 | CH03MAH |
| 1266 | 644099 | RTA22200004F.b.06.1.P.Seq | F | M00055743:312 | CH15CON |
| 1267 | 552614 | RTA22200008F.e.13.1.P.Seq | F | M00056344:73 | CH15CON |
| 1268 | 452523 | RTA22200007F.g.15.1.P.Seq | F | M00056203:810 | CH15CON |
| 1269 | 446789 | RTA22200004F.l.01.1.P.Seq | F | M00055807:710 | CH15CON |
| 1270 | 515631 | RTA22200010F.c.15.1.P.Seq | F | M00056369:412 | CH16COP |
| 1271 | 452523 | RTA22200009F.h.13.1.P.Seq | F | M00042805:88 | CH16COP |
| 1272 | 640116 | RTA22200007F.l.23.2.P.Seq | F | M00056250:61 | CH15CON |
| 1273 | 9113 | RTA22200230F.a.14.1.P.Seq | F | M00007097:47 | CH02COH |
| 1274 | 562221 | RTA22200006F.b.03.2.P.Seq | F | M00056001:59 | CH15CON |
| 1275 | 455972 | RTA22200012F.i.18.1.P.Seq | F | M00056729:44 | CH16COP |
| 1276 | 449137 | RTA22200009F.e.12.1.P.Seq | F | M00042771:13 | CH16COP |
| 1277 | 5078 | RTA22200013F.p.15.1.P.Seq | F | M00056914:29 | CH16COP |
| 1278 | 5078 | RTA22200013F.g.09.1.P.Seq | F | M00056844:57 | CH16COP |
| 1279 | 4016 | RTA22200228F.d.20.1.P.Seq | F | M00006807:712 | CH02COH |
| 1280 | 403111 | RTA22200005F.m.11.1.P.Seq | F | M00055946:77 | CH15CON |
| 1281 | 562292 | RTA22200023F.g.07.1.P.Seq | F | M00055053:23 | CH17COHLV |
| 1282 | 403111 | RTA22200007F.j.14.2.P.Seq | F | M00056226:612 | CH15CON |
| 1283 | 403111 | RTA22200007F.m.22.1.P.Seq | F | M00056262:28 | CH15CON |
| 1284 | 500959 | RTA22200006F.i.23.2.P.Seq | F | M00056066:87 | CH15CON |
| 1285 | 763 | RTA22200231F.g.24.1.P.Seq | F | M00007965:33 | CH03MAH |
| 1286 | 763 | RTA22200231F.h.01.1.P.Seq | F | M00007965:33 | CH03MAH |
| 1287 | 500959 | RTA22200008F.f.19.1.P.Seq | F | M00056456:19 | CH15CON |
| 1288 | 452071 | RTA22200001F.g.03.1.P.Seq | F | M00042700:85 | CH15CON |
| 1289 | 468672 | RTA22200026F.e.16.1.P.Seq | F | M00055472:62 | CH17COHLV |
| 1290 | 455492 | RTA22200001F.j.08.1.P.Seq | F | M00042738:410 | CH15CON |
| 1291 | 639667 | RTA22200015F.f.11.1.P.Seq | F | M00057079:59 | CH16COP |
| 1292 | 549829 | RTA22200024F.e.11.1.P.Seq | F | M00055181:51 | CH17COHLV |
| 1293 | 553158 | RTA22200021F.f.02.3.P.Seq | F | M00054752:17 | CH17COHLV |
| 1294 | 561485 | RTA22200026F.d.14.1.P.Seq | F | M00055421:311 | CH17COHLV |
| 1295 | 639352 | RTA22200002F.l.04.1.P.Seq | F | M00055528:66 | CH15CON |
| 1296 | 451401 | RTA22200018F.h.16.1.P.Seq | F | M00043317:64 | CH17COHLV |
| 1297 | 643103 | RTA22200001F.l.19.1.P.Seq | F | M00042893:38 | CH15CON |
| 1298 | 468736 | RTA22200001F.j.23.1.P.Seq | F | M00042746:62 | CH15CON |
| 1299 | 218416 | RTA22200004F.b.09.1.P.Seq | F | M00055744:38 | CH15CON |
| 1300 | 447501 | RTA22200002F.d.24.1.P.Seq | F | M00055453:51 | CH15CON |
| 1301 | 558371 | RTA22200024F.m.15.1.P.Seq | F | M00055242:44 | CH17COHLV |
| 1302 | 561794 | RTA22200015F.f.14.1.P.Seq | F | M00057080:32 | CH16C0P |
| 1303 | 645065 | RTA22200004F.b.15.1.P.Seq | F | M00055744:78 | CH15CON |
| 1304 | 451269 | RTA22200001F.b.05.1.P.Seq | F | M00042534:27 | CH15CON |
| 1305 | 401553 | RTA22200004F.e.16.1.P.Seq | F | M00055771:67 | CH15CON |
| 1306 | 555276 | RTA22200022F.n.12.1.P.Seq | F | M00054984:212 | CH17COHLV |
| 1307 | 551617 | RTA22200021F.f.21.3.P.Seq | F | M00054762:67 | CH17COHLV |
| 1308 | 463480 | RTA22200001F.i.08.1.P.Seq | F | M00042727:812 | CH15CON |
| 1309 | 549178 | RTA22200022F.n.07.1.P.Seq | F | M00054981:511 | CH17COHLV |
| 1310 | 374450 | RTA22200003F.d.02.1.P.Seq | F | M00055601:49 | CH15CON |
| 1311 | 562835 | RTA22200009F.o.07.1.P.Seq | F | M00042864:55 | CH16COP |
| 1312 | 730555 | RTA22200014F.j.23.2.P.Seq | F | M00056997:89 | CH16COP |
| 1313 | 732978 | RTA22200015F.f.09.1.P.Seq | F | M00057078:312 | CH16COP |
| 1314 | 1609 | RTA22200001F.k.16.1.P.Seq | F | M00042883:612 | CH15CON |
| 1315 | 18591 | RTA22200015F.c.09.1.P.Seq | F | M00057058:69 | CH16COP |
| 1316 | 553158 | RTA22200021F.f.12.3.P.Seq | F | M00054759:28 | CH17COHLV |
| 1317 | 470602 | RTA22200024F.i.03.1.P.Seq | F | M00055206:84 | CH17COHLV |
| 1318 | 639662 | RTA22200003F.d.14.1.P.Seq | F | M00055609:612 | CH15CON |
| 1319 | 644721 | RTA22200004F.p.13.1.P.Seq | F | M00055839:69 | CH15CON |
| 1320 | 453202 | RTA22200025F.n.08.2.P.Seq | F | M00055390:48 | CH17COHLV |
| 1321 | 554655 | RTA222ObO15F.e.19.1.P.Seq | F | M00057073:15 | CH16COP |
| 1322 | 641988 | RTA22200002F.c.20.1.P.Seq | F | M00055446:26 | CH15CON |
| 1323 | 453112 | RTA22200001F.f.24.1.P.Seq | F | M00042700:43 | CH15CON |
| 1324 | 550694 | RTA22200020F.c.09.1.P.Seq | F | M00054583:65 | CH17COHLV |
| 1325 | 649106 | RTA22200004F.h.02.1.P.Seq | F | M00055788:13 | CH15CON |
| 1326 | 638973 | RTA22200002F.g.14.1.P.Seq | F | M00055494:39 | CH15CON |
| 1327 | 549911 | RTA22200024F.h.16.1.P.Seq | F | M00055203:82 | CH17COHLV |
| 1328 | 648774 | RTA22200013F.o.21.1.P.Seq | F | M00056909:511 | CH16COP |
| 1329 | 549911 | RTA22200024F.g.03.1.P.Seq | F | M00055194:35 | CH17COHLV |
| 1330 | 639662 | RTA22200003F.a.10.1.P.Seq | F | M00055572:212 | CH15CON |
| 1331 | 560455 | RTA22200024F.l.03.1.P.Seq | F | M00055231:410 | CH17COHLV |
| 1332 | 735805 | RTA22200016F.d.23.1.P.Seq | F | M00057191:13 | CH15COP |
| 1333 | 732712 | RTA22200014F.p.03.2.P.Seq | F | M00057029:16 | CH16COP |
| 1334 | 446663 | RTA22200026F.f.10.1.P.Seq | F | M00055479:72 | CH17COHLV |
| 1335 | 226324 | RTA22200024F.e.17.1.P.Seq | F | M00055182:37 | CH17COHLV |
| 1336 | 453016 | RTA22200013F.m.14.1.P.Seq | F | M00056893:86 | CH16COP |
| 1337 | 550998 | RTA22200016F.d.20.1.P.Seq | F | M00057189:75 | CH16COP |
| 1338 | 452414 | RTA22200008F.d.21.1.P.Seq | F | M00056331:41 | CH15CON |
| 1339 | 129535 | RTA22200003F.a.13.1.P.Seq | F | M00055572:63 | CH15CON |
| 1340 | 447089 | RTA22200001F.c.04.1.P.Seq | F | M00042543:82 | CH15CON |
| 1341 | 447850 | RTA22200021F.c.07.2.P.Seq | F | M00054732:53 | CH17COHLV |
| 1342 | 556216 | RTA22200021F.l.05.3.P.Seq | F | M00054823:87 | CH17COHLV |
| 1343 | 452523 | RTA22200003F.d.13.1.P.Seq | F | M00055609:73 | CH15CON |
| 1344 | 44424 | RTA22200015F.i.23.1.P.Seq | F | M00057103:89 | CH16COP |
| 1345 | 648872 | RTA22200004F.m.05.1.P.Seq | F | M00055817:48 | CH15CON |
| 1346 | 451636 | RTA22200018F.e.12.1.P.Seq | F | M00043300:811 | CH17COHLV |
| 1347 | 5078 | RTA22200024F.a.14.1.P.Seq | F | M00055145:67 | CH17COHLV |
| 1348 | 403111 | RTA22200002F.g.21.1.P.Seq | F | M00055495:52 | CH15CON |
| 1349 | 648959 | RTA22200002F.d.02.1.P.Seq | F | M00055448:48 | CH15CON |
| 1350 | 380291 | RTA22200001F.b.12.1.P.Seq | F | M00042537:85 | CH15CON |
| 1351 | 380291 | RTA22200001F.c.09.1.P.Seq | F | M00042547:12 | CH15CON |
| 1352 | 230995 | RTA22200020F.e.24.1.P.Seq | F | M00054604:49 | CH17COHLV |
| 1353 | 562221 | RTA22200025F.a.22.1.P.Seq | F | M00055273:35 | CH17COHLV |
| 1354 | 450959 | RTA22200018F.f.14.1.P.Seq | F | M00043306:27 | CH17COHLV |
| 1355 | 452833 | RTA22200018F.d.24.1.P.Seq | F | M00042520:69 | CH17COHLV |
| 1356 | 550195 | RTA22200020F.e.19.1.P.Seq | F | M00054602:34 | CH17COHLV |
| 1357 | 448927 | RTA22200018F.l.10.1.P.Seq | F | M00043349:38 | CH17COHLV |
| 1358 | 551514 | RTA22200020F.h.08.1.P.Seq | F | M00054629:59 | CH17COHLV |
| 1359 | 549829 | RTA22200020F.f.14.1.P.Seq | F | M00054609:86 | CH17COHLV |
| 1360 | 551514 | RTA22200025F.f.12.1.P.Seq | F | M00055327:88 | CH17COHLV |
| 1361 | 561485 | RTA22200025F.i.07.1.P.Seq | F | M00055358:31 | CH17COHLV |
| 1362 | 453846 | RTA22200025F.k.20.1.P.Seq | F | M00055376:21 | CH17COHLV |
| 1363 | 69863 | RTA22200249F.j.23.1.P.Seq | F | M00027699:42 | CH17COHLV |
| 1364 | 727181 | RTA22200011F.d.23.1.P.Seq | F | M00056555:69 | CH16COP |
| 1365 | 454050 | RTA22200011F.c.06.1.P.Seq | F | M00056541:18 | CH16COP |
| 1366 | 725994 | RTA22200011F.b.07.1.P.Seq | F | M00056534:411 | CH16COP |
| 1367 | 1495 | RTA22200234F.j.11.1.P.Seq | F | M00022273:19 | CH03MAH |
| 1368 | 5665 | RTA2220b233F.n.01.1.P.Seq | F | M00021654:14 | CH03MAH |
| 1369 | 5665 | RTA22200233F.m.24.1.P.Seq | F | M00021654:14 | CH03MAH |
| 1370 | 646146 | RTA22200010F.i.03.1.P.Seq | F | M00056421:612 | CH16COP |
| 1371 | 8371 | RTA22200224F.e.18.1.P.Seq | F | M00005000:88 | CH02COH |
| 1372 | 73812 | RTA22200250F.e.14.1.P.Seq | F | M00027817:211 | CH04MAL |
| 1373 | 4242 | RTA22200233F.k.20.1.P.Seq | F | M00021620:610 | CH03MAH |
| 1374 | 5482 | RTA22200225F.a.01.1.P.Seq | F | M00005411:37 | CH02COH |
| 1375 | 5474 | RTA22200224F.p.13.1.P.Seq | F | M00005407:512 | CH02COH |
| 1376 | 5448 | RTA22200225F.a.13.1.P.Seq | F | M00005413:63 | CH02COH |
| 1377 | 7607 | RTA22200225F.c.10.1.P.Seq | F | M00005438:16 | CH02COH |
| 1378 | 555928 | RTA22200010F.o.23.1.P.Seq | F | M00056514:71 | CH16COP |
| 1379 | 4046 | RTA22200224F.j.04.1.P.Seq | F | M00005359:16 | CH02COH |
| 1380 | 554080 | RTA22200019F.f.02.1.P.Seq | F | M00043508:18 | CH17COHLV |
| 1381 | 451092 | RTA22200018F.p.13.1.P.Seq | F | M00043377:33 | CH17COHLV |
| 1382 | 551380 | RTA22200018F.p.22.1.P.Seq | F | M00043381:510 | CH17COHLV |
| 1383 | 546642 | RTA22200014F.o.20.1.P.Seq | F | M00057029:312 | CH16COP |
| 1384 | 1764 | RTA22200228F.c.06.1.P.Seq | F | M00006789:111 | CH02COH |
| 1385 | 650773 | RTA22200005F.c.05.1.P.Seq | F | M00055864:511 | CH15CON |
| 1386 | 644205 | RTA22200005F.d.20.1.P.Seq | F | M00055874:26 | CH15CON |
| 1387 | 185718 | RTA22200242F.m.05.1.P.Seq | F | M00027051:17 | CH04MAL |
| 1388 | 5538 | RTA22200227F.k.23.1.P.Seq | F | M00006712:62 | CH02COH |
| 1389 | 7546 | RTA22200222F.e.06.1.P.Seq | F | M00001625:28 | CH01COH |
| 1390 | 727789 | RTA22200014F.b.21.1.P.Seq | F | M00056939:68 | CH16C0P |
| 1391 | 3837 | RTA22200232F.m.13.1.P.Seq | F | M00022137:74 | CH03MAH |
| 1392 | 380477 | RTA22200007F.d.03.1.P.Seq | F | M00056162:46 | CH15CON |
| 1393 | 3299 | RTA22200238F.a.22.1.P.Seq | F | M00022861:210 | CH03MAH |
| 1394 | 448853 | RTA22200009F.b.12.2.P.Seq | F | M00042463:69 | CH16COP |
| 1395 | 736701 | RTA22200012F.h.21.1.P.Seq | F | M00056723:410 | CH16COP |
| 1396 | 735296 | RTA22200013F.k.05.1.P.Seq | F | M00056873:56 | CH16COP |
| 1397 | 13666 | RTA22200241F.f.02.1.P.Seq | F | M00026893:11 | CH04MAL |
| 1398 | 732712 | RTA22200013F.l.11.1.P.Seq | F | M00056883:17 | CH16COP |
| 1399 | 3765 | RTA22200227F.o.19.1.P.Seq | F | M00006755:14 | CH02COH |
| 1400 | 185596 | RTA22200243F.j.05.1.P.Seq | F | M00027134:72 | CH04MAL |
| 1401 | 1943 | RTA22200233F.e.19.1.P.Seq | F | M00008065:25 | CH03MAH |
| 1402 | 448193 | RTA22200009F.d.18.2.P.Seq | F | M00042764:24 | CH16COP |
| 1403 | 1793 | RTA22200235F.d.08.1.P.Seq | F | M00022444:311 | CH03MAH |
| 1404 | 2475 | RTA22200243F.c.22.1.P.Seq | F | M00027096:77 | CH04MAL |
| 1405 | 730866 | RTA22200012F.c.23.1.P.Seq | F | M00056690:69 | CH16COP |
| 1406 | 730389 | RTA22200013F.k.10.1.P.Seq | F | M00056874:86 | CH16COP |
| 1407 | 641884 | RTA22200003F.m.17.1.P.Seq | F | M00055705:77 | CH15CON |
| 1408 | 463487 | RTA22200014F.f.09.2.P.Seq | F | M00056961:28 | CH16COP |
| 1409 | 5156 | RTA22200225F.m.12.1.P.Seq | F | M00005516:86 | CH02COH |
| 1410 | 728408 | RTA22200012F.h.05.1.P.Seq | F | M00056718:72 | CH16COP |
| 1411 | 73812 | RTA22200242F.m.20.1.P.Seq | F | M00027054:23 | CH04MAL |
| 1412 | 1662 | RTA22200231F.n.18.1.P.Seq | F | M00007994:15 | CH03MAH |
| 1413 | 736556 | RTA22200012F.c.09.1.P.Seq | F | M00056683:29 | CH16COP |
| 1414 | 5240 | RTA22200230F.i.16.1.P.Seq | F | M00007172:33 | CH02COH |
| 1415 | 6184 | RTA22200230F.a.11.1.P.Seq | F | M00007096:82 | CH02COH |
| 1416 | 446404 | RTA22200007F.m.11.1.P.Seq | F | M00056253:86 | CH15CON |
| 1417 | 646825 | RTA22200005F.m.17.1.P.Seq | F | M00055951:32 | CH15CON |
| 1418 | 734929 | RTA22200011F.i.10.1.P.Seq | F | M00056592:47 | CH16COP |
| 1419 | 648851 | RTA22200007F.e.21.1.P.Seq | F | M00056185:13 | CH15CON |
| 1420 | 640135 | RTA22200014F.o.14.2.P.Seq | F | M00057027:76 | CH16COP |
| 1421 | 7443 | RTA22200234F.p.21.1.P.Seq | F | M00022411:712 | CH03MAH |
| 1422 | 454050 | RTA22200007F.h.03.1.P.Seq | F | M00056206:56 | CH15CON |
| 1423 | 3765 | RTA22200226F.l.11.1.P.Seq | F | M00005766:610 | CH02COH |
| 1424 | 648320 | RTA22200003F.p.03.1.P.Seq | F | M00055726:710 | CH15CON |
| 1425 | 451269 | RTA22200005F.g.17.1.P.Seq | F | M00055889:812 | CH15CON |
| 1426 | 535208 | RTA22200005F.h.10.1.P.Seq | F | M00055896:611 | CH15CON |
| 1427 | 728115 | RTA22200012F.i.04.1.P.Seq | F | M00056724:511 | CH16COP |
| 1428 | 5240 | RTA22200228F.b.08.1.P.Seq | F | M00006783:67 | CH02COH |
| 1429 | 909 | RTA22200233F.d.12.1.P.Seq | F | M00008043:211 | CH03MAH |
| 1430 | 447697 | RTA22200001F.m.18.1.P.Seq | F | M00042905:611 | CH15CON |
| 1431 | 447737 | RTA22200005F.k.16.1.P.Seq | F | M00055930:28 | CH15CON |
| 1432 | 651100 | RTA22200010F.d.03.1.P.Seq | F | M00056371:612 | CH16COP |
| 1433 | 735477 | RTA22200010F.d.20.1.P.Seq | F | M00056383:310 | CH16COP |
| 1434 | 3774 | RTA22200230F.c.04.1.P.Seq | F | M00007110:63 | CH02COH |
| 1435 | 646146 | RTA22200006F.a.09.2.P.Seq | F | M00055997:711 | CH15CON |
| 1436 | 643931 | RTA22200005F.j.20.1.P.Seq | F | M00055921:211 | CH15CON |
| 1437 | 463487 | RTA22200013F.h.14.1.P.Seq | F | M00056857:39 | CH16COP |
| 1438 | 650097 | RTA22200016F.h.08.1.P.Seq | F | M00057218:31 | CH16COP |
| 1439 | 554469 | RTA22200012F.o.20.1.P.Seq | F | M00056777:22 | CH16COP |
| 1440 | 476223 | RTA22200010F.a.21.1.P.Seq | F | M00056356:64 | CH16COP |
| 1441 | 8738 | RTA22200231F.c.10.1.P.Seq | F | M00007939:13 | CH03MAH |
| 1442 | 403978 | RTA22200008F.e.10.1.P.Seq | F | M00056342:73 | CH15CON |
| 1443 | 185539 | RTA22200240F.f.19.1.P.Seq | F | M00023377:39 | CH04MAL |
| 1444 | 451811 | RTA22200001F.p.04.1.P.Seq | F | M00054913:73 | CH15CON |
| 1445 | 140731 | RTA22200015F.n.18.1.P.Seq | F | M00057133:61 | CH16COP |
| 1446 | 734582 | RTA22200013F.c.12.1.P.Seq | F | M00056816:110 | CH16COP |
| 1447 | 463487 | RTA22200001F.e.05.1.P.Seq | F | M00042565:83 | CH15CON |
| 1448 | 558719 | RTA22200004F.d.17.1.P.Seq | F | M00055766:83 | CH15CON |
| 1449 | 21669 | RTA22200023F.j.14.1.P.Seq | F | M00055073:812 | CH17COHLV |
| 1450 | 470462 | RTA22200006F.b.13.2.P.Seq | F | M0005600:55 | CH15CON |
| 1451 | 3316 | RTA22200022F.i.09.1.P.Seq | F | M00054943:34 | CH17COHLV |
| 1452 | 553728 | RTA22200021F.e.07.3.P.Seq | F | M00054747:61 | CH17COHLV |
| 1453 | 736014 | RTA22200016F.p.01.1.P.Seq | F | M00057279:72 | CH16COP |
| 1454 | 237288 | RTA22200002F.m.12.1.P.Seq | F | M00055538:811 | CH15CON |
| 1455 | 11141 | RTA22200226F.m.20.1.P.Seq | F | M00005782:210 | CH02COH |
| 1456 | 556421 | RTA22200022F.k.11.1.P.Seq | F | M00054957:22 | CH17COHLV |
| 1457 | 549435 | RTA22200006F.h.16.1.P.Seq | F | M00056055:43 | CH15CON |
| 1458 | 448927 | RTA22200009F.g.08.1.P.Seq | F | M00042792:710 | CH16COP |
| 1459 | 379105 | RTA22200023F.o.09.1.P.Seq | F | M0005512:37 | CH17COHLV |
| 1460 | 552614 | RTA22200020F.m.20.1.P.Seq | F | M00054680:56 | CH17COHLV |
| 1461 | 470602 | RTA22200004F.l.04.1.P.Seq | F | M00055807:34 | CH15CON |
| 1462 | 557039 | RTA22200022F.o.03.1.P.Seq | F | M00054988:72 | CH17COHLV |
| 1463 | 549864 | RTA22200022F.k.06.1.P.Seq | F | M00054954:33 | CH17COHLV |
| 1464 | 449836 | RTA22200017F.a.23.1.P.Seq | F | M00057301:812 | CH16COP |
| 1465 | 554812 | RTA22200023F.m.06.1.P.Seq | F | M00055094:89 | CH17COHLV |
| 1466 | 3316 | RTA22200021F.l.17.3.P.Seq | F | M00054827:81 | CH17COHLV |
| 1467 | 649852 | RTA22200003F.l.09.1.P.Seq | F | M00055689:712 | CH15CON |
| 1468 | 453592 | RTA22200001F.g.17.1.P.Seq | F | M00042705:42 | CH15CON |
| 1469 | 455096 | RTA22200026F.g.02.1.P.Seq | F | M00055482:11 | CH17COHLV |
| 1470 | 446199 | RTA22200009F.n.08.1.P.Seq | F | M00042855:29 | CH16COP |
| 1471 | 558427 | RTA22200024F.m.11.1.P.Seq | F | M00055242:56 | CH17COHLV |
| 1472 | 450255 | RTA22200021F.j.13.3.P.Seq | F | M00054810:82 | CH17COHLV |
| 1473 | 452026 | RTA22200003F.l.10.1.P.Seq | F | M00055689:62 | CH15CON |
| 1474 | 374971 | RTA22200024F.p.11.1.P.Seq | F | M00055260:612 | CH17COHLV |
| 1475 | 446404 | RTA22200003F.l.04.1.P.Seq | F | M00055686:511 | CH15CON |
| 1476 | 549591 | RTA22200025F.p.19.2.P.Seq | F | M00055404:37 | CH17COHLV |
| 1477 | 640135 | RTA22200014F.o.14.3.P.Seq | F | M00057027:76 | CH16COP |
| 1478 | 646248 | RTA22200016F.k.06.1.P.Seq | F | M00057239:78 | CH16COP |
| 1479 | 639705 | RTA22200004F.e.05.1.P.Seq | F | M00055770:71 | CH15CON |
| 1480 | 483084 | RTA22200004F.m.03.1.P.Seq | F | M00055817:61 | CH15CON |
| 1481 | 464029 | RTA22200026F.b.04.1.P.Seq | F | M00055408:63 | CH17COHLV |
| 1482 | 428005 | RTA22200021F.c.03.2.P.Seq | F | M00054731:312 | CH17COHLV |
| 1483 | 91178 | RTA22200024F.o.02.1.P.Seq | F | M00055252:32 | CH17COHLV |
| 1484 | 550571 | RTA22200024F.k.21.1.P.Seq | F | M00055227:57 | CH17COHLV |
| 1485 | 735028 | RTA22200015F.e.10.1.P.Seq | F | M00057070:28 | CH16COP |
| 1486 | 559409 | RTA22200021F.m.17.2.P.Seq | F | M00054839:66 | CH17COHLV |
| 1487 | 551172 | RTA22200021F.m.09.2.P.Seq | F | M00054836:25 | CH17COHLV |
| 1488 | 648872 | RTA22200002F.g.23.1.P.Seq | F | M00055496:69 | CH15CON |
| 1489 | 446404 | RTA22200001F.l.13.1.P.Seq | F | M00042887:47 | CH15CON |
| 1490 | 734063 | RTA22200016F.j.24.1.P.Seq | F | M00057237:211 | CH16COP |
| 1491 | 467991 | RTA22200008F.c.22.1.P.Seq | F | M00056320:82 | CH15CON |
| 1492 | 454050 | RTA22200008F.d.06.1.P.Seq | F | M00056323.312 | CH16COP |
| 1493 | 734646 | RTA22200015F.j.06.1.P.Seq | F | M00057104:68 | CH16COP |
| 1494 | 450192 | RTA22200018F.n.06.1.P.Seq | F | M00043361:45 | CH17COHLV |
| 1495 | 403978 | RTA22200018F.f.01.1.P.Seq | F | M00043304:35 | CH17COHLV |
| 1496 | 734209 | RTA22200016F.c.16.1.P.Seq | F | M00057174:36 | CH16COP |
| 1497 | 14805 | RTA22200021F.o.12.2.P.Seq | F | M00054856:12 | CH17COHLV |
| 1498 | 230995 | RTA22200020F.f.01.1.P.Seq | F | M00054604:49 | CH17COHLV |
| 1499 | 120049 | RTA22200020F.e.18.1.P.Seq | F | M00054601:58 | CH17COHLV |
| 1500 | 642142 | RTA22200004F.f.16.1.P.Seq | F | M00055780:27 | CH15CON |
| 1501 | 403978 | RTA22200018F.e.24.1.P.Seq | F | M00043304:35 | CH17COHLV |
| 1502 | 386543 | RTA22200018F.g.02.1.P.Seq | F | M00043309:87 | CH17COHLV |
| 1503 | 379105 | RTA22200018F.a.14.1.P.Seq | F | M00042355:72 | CH17COHLV |
| 1504 | 450255 | RTA22200018F.c.16.1.P.Seq | F | M00042455:411 | CH17COHLV |
| 1505 | 730143 | RTA22200011F.a.03.1.P.Seq | F | M00056526:511 | CH16COP |
| 1506 | 734209 | RTA22200010F.m.02.1.P.Seq | F | M00056496:11 | CH16COP |
| 1507 | 401553 | RTA22200010F.m.16.1.P.Seq | F | M00056501:87 | CH16COP |
| 1508 | 72979 | RTA22200019F.a.15.1.P.Seq | F | M00043388:39 | CH17COHLV |
| 1509 | 726307 | RTA22200010F.o.16.1.P.Seq | F | M00056512:312 | CH16COP |
| 1510 | 230995 | RTA22200019F.b.14.1.P.Seq | F | M00043397:29 | CH17COHLV |
| 1511 | 35241 | RTA22200227F.f.19.1.P.Seq | F | M00006650:13 | CH02COH |
| 1512 | 8112 | RTA22200237F.m.13.1.P.Seq | F | M00022823:43 | CH03MAH |
| 1513 | 5240 | RTA22200237F.m.05.1.P.Seq | F | M00022820:36 | CH03MAH |
| 1514 | 447326 | RTA22200005F.c.22.1.P.Seq | F | M00055868:69 | CH15CON |
| 1515 | 2676 | RTA22200227F.k.01.1.P.Seq | F | M00006695:88 | CH02COH |
| 1516 | 736701 | RTA22200014F.e.11.1.P.Seq | F | M00056955:79 | CH16COP |
| 1517 | 736701 | RTA22200014F.e.11.2.P.Seq | F | M00056955:79 | CH16COP |
| 1518 | 8371 | RTA22200229F.a.07.1.P.Seq | F | M00006921:88 | CH02COH |
| 1519 | 8336 | RTA22200235F.f.24.1.P.Seq | F | M00022470:71 | CH03MAH |
| 1520 | 185542 | RTA22200248F.h.05.1.P.Seq | F | M00027564:43 | CH04MAL |
| 1521 | 448046 | RTA22200014F.d.13.1.P.Seq | F | M00056951:69 | CH16COP |
| 1522 | 185422 | RTA22200242F.j.15.1.P.Seq | F | M00027034:411 | CH04MAL |
| 1523 | 650448 | RTA22200005F.c.17.1.P.Seq | F | M00055868:66 | CH15CON |
| 1524 | 5753 | RTA22200227F.n.02.1.P.Seq | F | M00006737:111 | CH02COH |
| 1525 | 8001 | RTA22200222F.a.01.1.P.Seq | F | M00001340:51 | CH01COH |
| 1526 | 1644 | RTA22200240F.j.17.1.P.Seq | F | M00023430:410 | CH04MAL |
| 1527 | 4453 | RTA22200237F.o.18.1.P.Seq | F | M00022836:73 | CH03MAH |
| 1528 | 454152 | RTA22200020F.j.08.1.P.Seq | F | M00054646:21 | CH17COHLV |
| 1529 | 9913 | RTA22200231F.m.19.1.P.Seq | F | M00007991:71 | CH03MAH |
| 1530 | 1350 | RTA22200227F.m.14.1.P.Seq | F | M00006731:51 | CH02COH |
| 1531 | 188 | RTA22200244F.i.18.1.P.Seq | F | M00027194:15 | CH04MAL |
| 1532 | 4471 | RTA22200232F.g.09.1.P.Seq | F | M00022025:42 | CH03MAH |
| 1533 | 2622 | RTA22200248F.i.16.1.P.Seq | F | M00027573:69 | CH04MAL |
| 1534 | 185465 | RTA22200248F.j.08.1.P.Seq | F | M00027578:54 | CH04MAL |
| 1535 | 19205 | RTA22200244F.b.14.1.P.Seq | F | M00027171:27 | CH04MAL |
| 1536 | 185635 | RTA22200242F.l.11.1.P.Seq | F | M00027046:55 | CH04MAL |
| 1537 | 5289 | RTA22200235F.o.06.2.P.Seq | F | M00022559:49 | CH03MAH |
| 1538 | 779 | RTA22200238F.f.01.1.P.Seq | F | M00022901:511 | CH03MAH |
| 1539 | 779 | RTA22200238F.e.24.1.P.Seq | F | M00022901:511 | CH03MAH |
| 1540 | 5289 | RTA22200235F.m.20.2.P.Seq | F | M00022550:24 | CH03MAH |
| 1541 | 456808 | RTA22200013F.d.22.1.P.Seq | F | M00056823:17 | CH16COP |
| 1542 | 646620 | RTA22200007F.n.14.1.P.Seq | F | M00056267:22 | CH15CON |
| 1543 | 546642 | RTA22200013F.d.21.1.P.Seq | F | M00056823:25 | CH16COP |
| 1544 | 649732 | RTA22200003F.n.10.1.P.Seq | F | M00055716:24 | CH15CON |
| 1545 | 5240 | RTA22200231F.f.05.1.P.Seq | F | M00007953:89 | CH03MAH |
| 1546 | 448046 | RTA22200009F.c.06.2.P.Seq | F | M00042511:14 | CH16COP |
| 1547 | 650476 | RTA22200008F.f.07.1.P.Seq | F | M00056447:14 | CH15CON |
| 1548 | 379341 | RTA22200016F.n.21.1.P.Seq | F | M00057272:84 | CH16COP |
| 1549 | 401849 | RTA22200003F.p.02.1.P.Seq | F | M00055726:412 | CH15CON |
| 1550 | 11452 | RTA22200227F.f.18.1.P.Seq | F | M00006649:211 | CH02COH |
| 1551 | 185417 | RTA22200242F.b.12.1.P.Seq | F | M00026977:59 | CH04MAL |
| 1552 | 4471 | RTA22200232F.c.20.1.P.Seq | F | M00021925:810 | CH03MAH |
| 1553 | 2557 | RTA22200228F.e.11.1.P.Seq | F | M00006810:411 | CH02COH |
| 1554 | 3656 | RTA22200227F.f.01.1.P.Seq | F | M00006641:83 | CH02COH |
| 1555 | 2327 | RTA22200241F.i.20.1.P.Seq | F | M00026917:83 | CH04MAL |
| 1556 | 449026 | RTA22200009F.o.17.1.P.Seq | F | M00042867:81 | CH16COP |
| 1557 | 730227 | RTA22200013F.n.03.1.P.Seq | F | M00056896:610 | CH16COP |
| 1558 | 650864 | RTA22200006F.e.08.2.P.Seq | F | M00056028:63 | CH15CON |
| 1559 | 530774 | RTA22200004F.j.19.1.P.Seq | F | M00055803:111 | CH15CON |
| 1560 | 395341 | RTA22200011F.g.06.1.P.Seq | F | M00056580:610 | CH16COP |
| 1561 | 557906 | RTA22200004F.a.07.1.P.Seq | F | M00055735:75 | CH15CON |
| 1562 | 452531 | RTA22200009F.l.08.2.P.Seq | F | M00042842:58 | CH16COP |
| 1563 | 559057 | RTA22200021F.e.09.3.P.Seq | F | M00054747:36 | CH17COHLV |
| 1564 | 448046 | RTA22200014F.d.13.2.P.Seq | F | M00056951:69 | CH16COP |
| 1565 | 553547 | RTA22200022F.g.22.1.P.Seq | F | M00054935:51 | CH17COHLV |
| 1566 | 4636 | RTA22200231F.e.20.1.P.Seq | F | M00007953:11 | CH03MAH |
| 1567 | 455601 | RTA22200006F.f.06.2.P.Seq | F | M00056037:22 | CH15CON |
| 1568 | 172013 | RTA22200002F.c.05.1.P.Seq | F | M00055441:42 | CH15CON |
| 1569 | 552597 | RTA22200013F.a.02.1.P.Seq | F | M00056791:612 | CH16COP |
| 1570 | 446531 | RTA22200001F.c.11.1.P.Seq | F | M00042547:411 | CH15CON |
| 1571 | 639352 | RTA22200008F.a.09.1.P.Seq | F | M00056293:79 | CH15CON |
| 1572 | 642604 | RTA22200004F.e.12.1.P.Seq | F | M00055771:65 | CH15CON |
| 1573 | 558534 | RTA22200008F.c.04.1.P.Seq | F | M00056310:76 | CH15CON |
| 1574 | 556421 | RTA22200026F.b.02.1.P.Seq | F | M00055408:59 | CH17COHLV |
| 1575 | 735477 | RTA22200016F.k.10.1.P.Seq | F | M00057242:67 | CH16COP |
| 1576 | 640703 | RTA22200014F.o.06.2.P.Seq | F | M00057024:52 | CH16COP |
| 1577 | 643878 | RTA22200015F.j.14.1.P.Seq | F | M00057106:13 | CH16COP |
| 1578 | 557797 | RTA22200004F.e.07.1.P.Seq | F | M00055771:111 | CH15CON |
| 1579 | 557200 | RTA22200021F.b.08.2.P.Seq | F | M00054727:510 | CH17COHLV |
| 1580 | 729531 | RTA22200015F.h.06.1.P.Seq | F | M00057091:512 | CH16COP |
| 1581 | 734554 | RTA22200014F.o.09.2.P.Seq | F | M00057024:88 | CH16COP |
| 1582 | 418008 | RTA22200020F.e.16.1.P.Seq | F | M00054601:810 | CH17COHLV |
| 1583 | 558614 | RTA22200024F.k.16.1.P.Seq | F | M00055227:89 | CH17COHLV |
| 1584 | 452245 | RTA22200025F.l.16.1.P.Seq | F | M00055381:59 | CH17COHLV |
| 1585 | 449891 | RTA22200019F.b.13.1.P.Seq | F | M00043397:22 | CH17COHLV |
| 1586 | 547916 | RTA22200011F.a.18.1.P.Seq | F | M00056529:612 | CH16COP |
| 1587 | 6162 | RTA22200233F.l.19.1.P.Seq | F | M00021628:211 | CH03MAH |
| 1588 | 6162 | RTA22200234F.f.14.1.P.Seq | F | M00022234:45 | CH03MAH |
| 1589 | 4809 | RTA22200224F.d.21.1.P.Seq | F | M00004868:412 | CH02COH |
| 1590 | 3926 | RTA22200242F.d.18.1.P.Seq | F | M00026993:86 | CH04MAL |
| 1591 | 185693 | RTA22200248F.d.20.1.P.Seq | F | M00027532:32 | CH04MAL |
| 1592 | 641683 | RTA22200007F.m.16.1.P.Seq | F | M00056256:811 | CH15CON |
| 1593 | 11351 | RTA22200226F.o.18.1.P.Seq | F | M00005818:29 | CH02COH |
| 1594 | 650864 | RTA22200008F.g.16.1.P.Seq | F | M00056467:57 | CH15CON |
| 1595 | 460445 | RTA22200001F.i.11.1.P.Seq | F | M00042728:57 | CH15CON |
| 1596 | 447669 | RTA22200011F.e.16.1.P.Seq | F | M00056561:48 | CH16COP |
| 1597 | 227936 | RTA22200016F.m.13.1.P.Seq | F | M00057259:810 | CH16COP |
| 1598 | 639459 | RTA22200002F.l.20.1.P.Seq | F | M00055532:78 | CH15CON |
| 1599 | 650195 | RTA22200004F.a.04.1.P.Seq | F | M00055734:85 | CH15CON |
| 1600 | 734793 | RTA22200016F.n.06.1.P.Seq | F | M00057268:510 | CH16COP |
| 1601 | 540787 | RTA22200002F.m.10.1.P.Seq | F | M00055536:56 | CH15CON |
| 1602 | 400654 | RTA22200003F.n.13.1.P.Seq | F | M00055718:65 | CH15CON |
| 1603 | 731467 | RTA22200016F.f.21.1.P.Seq | F | M00057208:36 | CH16COP |
| 1604 | 4045 | RTA22200232F.g.23.1.P.Seq | F | M00022052:18 | CH03MAH |
| 1605 | 447669 | RTA22200011F.l.23.1.P.Seq | F | M00056628:61 | CH16COP |
| 1606 | 11351 | RTA22200230F.c.20.1.P.Seq | F | M00007121:78 | CH02COH |
| 1607 | 648931 | RTA22200002F.l.16.1.P.Seq | F | M00055530:27 | CH15CON |
| 1608 | 726786 | RTA22200016F.e.08.1.P.Seq | F | M00057192:72 | CH16COP |
| 1609 | 4508 | RTA22200226F.o.02.1.P.Seq | F | M00005810:710 | CH02COH |
| 1610 | 415058 | RTA22200022F.h.07.1.P.Seq | F | M00054937:23 | CH17COHLV |
| 1611 | 450633 | RTA22200006F.l.11.2.P.Seq | F | M00056085:811 | CH15SCON |
| 1612 | 736955 | RTA22200014F.h.05.2.P.Seq | F | M00056973:28 | CH16COP |
| 1613 | 729851 | RTA22200013F.h.15.1.P.Seq | F | M00056858:23 | CH16COP |
| 1614 | 2512 | RTA22200235F.c.11.1.P.Seq | F | M00022430:410 | CH03MAH |
| 1615 | 452704 | RTA22200008F.f.21.1.P.Seq | F | M00056456:61 | CH15CON |
| 1616 | 4589 | RTA22200230F.c.15.1.P.Seq | F | M00007117:83 | CH02COH |
| 1617 | 4727 | RTA22200233F.e.16.1.P.Seq | F | M00008059:28 | CH03MAH |
| 1618 | 454380 | RTA22200014F.d.23.1.P.Seq | F | M00056953:16 | CH16COP |
| 1619 | 553912 | RTA22200021F.j.04.3.P.Seq | F | M00054808:47 | CH17COHLV |
| 1620 | 450004 | RTA22200012F.k.08.1.P.Seq | F | M00058741:36 | CH16COP |
| 1621 | 448193 | RTA22200012F.g.20.1.P.Seq | F | M00056715:34 | CH16COP |
| 1622 | 549591 | RTA22200020F.i.03.1.P.Seq | F | M00054636:62 | CH17COHLV |
| 1623 | 448511 | RTA22200023F.b.05.1.P.Seq | F | M00055021:411 | CH17COHLV |
| 1624 | 335 | RTA22200012F.l.18.1.P.Seq | F | M00056753:110 | CH16COP |
| 1625 | 561382 | RTA22200026F.c.05.1.P.Seq | F | M00055413:27 | CH17COHLV |
| 1626 | 3447 | RTA22200227F.k.15.1.P.Seq | F | M00006705:19 | CH02COH |
| 1627 | 639896 | RTA22200012F.c.15.1.P.Seq | F | M00056684:611 | CH16COP |
| 1628 | 1353 | RTA22200241F.e.20.1.P.Seq | F | M00026890:42 | CH02COH |
| 1629 | 3031 | RTA22200229F.i.23.1.P.Seq | F | M00006996:610 | CH02COH |
| 1630 | 557928 | RTA22200021F.g.03.3.P.Seq | F | M00054765:35 | CH17COHLV |
| 1631 | 4727 | RTA22200237F.n.09.1.P.Seq | F | M00022829:810 | CH03MAH |
| 1632 | 4046 | RTA22200226F.g.16.1.P.Seq | F | M00005655:68 | CH02COH |
| 1633 | 10882 | RTA22200241F.b.11.1.P.Seq | F | M00926861:19 | CH04MAL |
| 1634 | 646283 | RTA22200007F.c.22.1.P.Seq | F | M00056161:74 | CH15CON |
| 1635 | 646283 | RTA22200002F.d.05.1.P.Seq | F | M00055448:57 | CH15CON |
| 1636 | 139516 | RTA22200242F.e.16.1.P.Seq | F | M00007131:11 | CH02COH |
| 1637 | 6184 | RTA22200230F.e.13.1.P.Seq | F | M00007131:11 | CH02COH |
| 1638 | 6184 | RTA22200228F.c.12.1.P.Seq | F | M00006795:13 | CH02COH |
| 1639 | 454653 | RTA22200013F.e.17.1.P.Seq | F | M00056830:72 | CH16COP |
| 1640 | 3309 | RTA22200232F.c.11.1.P.Seq | F | M00021911:83 | CH03MAH |
| 1641 | 1037 | RTA22200248F.e..02.1P.Seq | F | M00027535:58 | CH04MAL |
| 1642 | 450665 | RTA22200002F.g.06.1.P.Seq | F | M00055491:78 | CH15CON |
| 1643 | 726307 | RTA22200016F.b.16.1.P.Seq | F | M00057167:512 | CH16COP |
| 1644 | 447669 | RTA22200001F.b.14.1.P.Seq | F | M00042538:56 | CH15CON |
| 1645 | 639651 | RTA22200003F.b.15.1.P.Seq | F | M00055584:76 | CH15CON |
| 1646 | 736860 | RTA22200015F.b.11.1.P.Seq | F | M00057049:76 | CH16COP |
| 1647 | 553705 | RTA22200025F.j.22.1.P.Seq | F | M00055371:61 | CH17COHLV |
| 1648 | 451375 | RTA22200018F.j.20.1.P.Seq | F | M00043336:58 | CH17COHLV |
| 1649 | 204862 | RTA22200006F.h.24.2.P.Seq | F | M00056058:84 | CH15CON |
| 1650 | 530883 | RTA22200011F.o.17.1.P.Seq | F | M00056652:64 | CH16COP |
| 1651 | 447539 | RTA22200001F.e.22.1.P.Seq | F | M00042575:41 | CH15CON |
| 1652 | 455096 | RTA22200020F.m.23.1.P.Seq | F | M00054680:211 | CH17COHLV |
| 1653 | 8336 | RTA22200235F.g.01.1.P.Seq | F | M00022470:71 | CH03MAH |
| 1654 | 449142 | RTA22200006F.f.09.2.P.Seq | F | M00056041:34 | CH15CON |
| 1655 | 557401 | RTA22200003F.j.04.1.P.Seq | F | M00055668:27 | CH15CON |
| 1656 | 418763 | RTA22200002F.e.07.1.P.Seq | F | M00055454:75 | CH15SCON |
| 1657 | 17649 | RTA22200238F.b.23.1.P.Seq | F | M00022876:48 | CH03MAH |
| 1658 | 2078 | RTA22200238F.h.11.1.P.Seq | F | M00022928:63 | CH03MAH |
| 1659 | 640370 | RTA22206010F.g.14.1.P.Seq | F | M00056410:54 | CH16COP |
| 1660 | 449269 | RTA22200005F.h.22.1.P.Seq | F | M00055906:410 | CH15CON |
| 1661 | 639029 | RTA22200006F.o.10.2.P.Seq | F | M00056114:36 | CH15CON |
| 1662 | 448677 | RTA22200023F.l.07.1.P.Seq | F | M00055088:59 | CH17COHLV |
| 1663 | 349 | RTA22200233F.a.09.1.P.Seq | F | M00008014:21 | CH03MAH |
| 1664 | 447494 | RTA22200002F.e.05.1.P.Seq | F | M00055454:811 | CH15CON |
| 1665 | 551433 | RTA22200021F.k.12.3.P.Seq | F | M00054816:411 | CH17COHLV |
| 1666 | 414739 | RTA22200019F.f.07.1.P.Seq | F | M00043508:31 | CH17COHLV |
| 1667 | 640525 | RTA22200002F.n.01.1.P.Seq | F | M00055543:35 | CH15CON |
| 1668 | 640525 | RTA22200002F.m.24.1.P.Seq | F | M00055543:35 | CH15CON |
| 1669 | 233108 | RTA22200002F.p.10.1.P.Seq | F | M00055560:212 | CH15CON |
| 1670 | 643594 | RTA22200003F.l.06.1.P.Seq | F | M00055687:61 | CH15CON |
| 1671 | 1642 | RTA22200231F.a.18.1.P.Seq | F | M00007931:110 | CH03MAH |
| 1672 | 643804 | RTA22200003F.h.09.1.P.Seq | F | M00055656:59 | CH15CON |
| 1673 | 449701 | RTA22200012F.h.22.1.P.Seq | F | M00056723:311 | CH16COP |
| 1674 | 185695 | RTA22200242F.c.21.1.P.Seq | F | M00026986:810 | CH04MAL |
| 1675 | 555830 | RTA22200003F.k.13.1.P.Seq | F | M00055679:17 | CH15CON |
| 1676 | 227936 | RTA22200010F.n.11.1.P.Seq | F | M00056505:47 | CH16COP |
| 1677 | 1609 | RTA22200226F.g.09.1.P.Seq | F | M00005650:16 | CH02COH |
| 1678 | 643938 | RTA22200005F.o.22.1.P.Seq | F | M00055980:110 | CH15CON |
| 1679 | 3656 | RTA22200227F.e.24.1.P.Seq | F | M00006641:83 | CH02COH |
| 1680 | 16576 | RTA22200248F.e.07.1.P.Seq | F | M00027536:712 | CH04MAL |
| 1681 | 9784 | RTA22200243F.c.16.1.P.Seq | F | M00027093:28 | CH04MAL |
| 1682 | 2557 | RTA22200228F.p.12.2.P.Seq | F | M00006917:48 | CH02COH |
| 1683 | 4620 | RTA22200237F.o.15.1.P.Seq | F | M00022834:71 | CH03MAH |
| 1684 | 43642 | RTA22200021F.p.23.2.P.Seq | F | M00054865:84 | CH17COHLV |
| 1685 | 555103 | RTA22200016F.l.08.1.P.Seq | F | M00005724:36 | CH16COP |
| 1686 | 643341 | RTA22200002F.g.16.1.P.Seq | F | M00055495:72 | CH15CON |
| 1687 | 185531 | RTA22200248F.l.12.1.P.Seq | F | M00027588:31 | CH04MAL |
| 1688 | 4045 | RTA22200224F.b.17.1.P.Seq | F | M00004842:27 | CH02COH |
| 1689 | 400258 | RTA22200011F.e.10.1.P.Seq | F | M00056557:42 | CH16COP |
| 1690 | 96618 | RTA22200248F.j.22.1.P.Seq | F | M00027581:51 | CH04MAL |
| 1691 | 646060 | RTA22200007F.p.23.1.P.Seq | F | M00056291:71 | CH15CON |
| 1692 | 5665 | RTA22200232F.a.17.1.P.Seq | F | M00021854:57 | CH03MAH |
| 1693 | 149265 | RTA22200241F.o.03.1.P.Seq | F | M00026951:43 | CH04MAL |
| 1694 | 727314 | RTA22200012F.g.14.1.P.Seq | F | M00056714:86 | CH16COP |
| 1695 | 736349 | RTA22200014F.d.03.1.P.Seq | F | M00056947:69 | CH16COP |
| 1996 | 648931 | RTA22200006F.k.13.2.P.Seq | F | M00056081:25 | CH15CON |
| 1697 | 553881 | RTA22200003F.m.10.1.P.Seq | F | M00055703:79 | CH15CON |
| 1698 | 7444 | RTA22200235F.d.02.1.P.Seq | F | M00022440:41 | CH03MAH |
| 1699 | 150 | RTA22200235F.p.14.2.P.Seq | F | M00022571:411 | CH03MAH |
| 1700 | 2889 | RTA22200228F.p.09.2.P.Seq | F | M00006917:15 | CH02COH |
| 1701 | 730670 | RTA22200013F.a.09.1.P.Seq | F | M00056793:87 | CH16COP |
| 1702 | 560984 | RTA22200021F.n.20.2.P.Seq | F | M00054851:53 | CH17COHLV |
| 1703 | 453708 | RTA22200026F.e.10.1.P.Seq | F | M00055425:35 | CH17COHLV |
| 1704 | 48977 | RTA22200024F.p.08.1.P.Seq | F | M00055259:64 | CH17COHLV |
| 1705 | 547916 | RTA22200008F.g.01.1.P.Seq | F | M00056459:37 | CH15CON |
| 1706 | 547916 | RTA22200008F.f.24.1.P.Seq | F | M00056459:37 | CH15CON |
| 1707 | 97507 | RTA22200023F.p.11.1.P.Seq | F | M00055134:82 | CH17COHLV |
| 1708 | 735966 | RTA22200012F.n.08.1.P.Seq | F | M00056766:110 | CH16COP |
| 1709 | 35 | RTA22200012F.m.20.1.P.Seq | F | M00056760:14 | CH16COP |
| 1710 | 650195 | RTA22200002F.m.11.1.P Seq | F | M00055536:63 | CH15CON |
| 1711 | 639705 | RTA22200002F.i.11.1.P.Seq | F | M00055510:27 | CH15CON |
| 1712 | 185465 | RTA22200013F.c.17.1.P.Seq | F | M00056817:33 | CH16COP |
| 1713 | 378525 | RTA22200015F.h.03.1.P.Seq | F | M00057091:33 | CH16COP |
| 1714 | 2889 | RTA22200228F.p.10.2.P.Seq | F | M00006917:14 | CH02COH |
| 1715 | 557686 | RTA22200025F.f.19.1.P.Seq | F | M00055334:89 | CH17COHLV |
| 1716 | 735786 | RTA22200010F.h.02.1.P.Seq | F | M00056414:15 | CH16COP |
| 1717 | 455145 | RTA22200003F.f.10.1.P.Seq | F | M00055636:610 | CH15CON |
| 1718 | 639667 | RTA22200002F.k.13.1.P.Seq | F | M00055525:27 | CH15CON |
| 1719 | 446913 | RTA22200001F.m.21.1.P.Seq | F | M00042905:310 | CH15CON |
| 1720 | 402494 | RTA22200014F.c.09.2.P.Seq | F | M00056941:52 | CH16COP |
| 1721 | 734256 | RTA22200014F.l.11.1.P.Seq | F | M00057005:43 | CH16COP |
| 1722 | 734256 | RTA22200012F.g.05.1.P.Seq | F | M00056711:15 | CH16COP |
| 1723 | 559362 | RTA22200023F.n.21.1.P.Seq | F | M00055115:55 | CH17COHLV |
| 1724 | 639651 | RTA22200003F.c.22.1.P.Seq | F | M00055597:58 | CH15CON |
| 1725 | 419774 | RTA22200008F.g.11.1.P.Seq | F | M00056466:52 | CH15CON |
| 1726 | 555318 | RTA22200025F.a.17.1.P.Seq | F | M00055271:35 | CH17COHLV |
| 1727 | 449956 | RTA22200010F.b.08.1.P.Seq | F | M00056359:111 | CH16COP |
| 1728 | 558427 | RTA22200015F.p.12.1.P.Seq | F | M00057146:39 | CH16COP |
| 1729 | 7531 | RTA22200225F.j.13.1.P.Seq | F | M00005491:812 | CH02COH |
| 1730 | 446514 | RTA22200001F.a.16.1.P.Seq | F | M00042527:47 | CH15CON |
| 1731 | 456808 | RTA22200010F.o.15.1.P.Seq | F | M00056512:59 | CH16COP |
| 1732 | 447035 | RTA22200003F.f.08.1.P.Seq | F | M00055635:510 | CH15CON |
| 1733 | 446913 | RTA22200001F.j.16.1.P.Seq | F | M00042742:16 | CH15CON |
| 1734 | 446900 | RTA22200022F.g.20.1.P.Seq | F | M00054934:43 | CH17COHLV |
| 1735 | 504513 | RTA22200003F.f.07.1.P.Seq | F | M00055635:810 | CH15CON |
| 1736 | 380477 | RTA22200014F.j.09.1.P.Seq | F | M00056993:26 | CH16COP |
| 1737 | 1213 | RTA22200244F.p.13.1.P.Seq | F | M00027231:13 | CH04MAL |
| 1738 | 8259 | RTA22200222F.J.14.1.P.Seq | F | M00004093:310 | CH01COH |
| 1739 | 8259 | RTA22200222F.h.23.1.P.Seq | F | M00003922:83 | CH01COH |
| 1740 | 552968 | RTA22200019F.d.20.1.P.Seq | F | M00043503:38 | CH17COHLV |
| 1741 | 650845 | RTA22200010F.o.09.1.P.Seq | F | M00056511:812 | CH16COP |
| 1742 | 648594 | RTA22200014F.h.17.1.P.Seq | F | M00056977:73 | CH16COP |
| 1743 | 648594 | RTA22200014F.h.17.2.P.Seq | F | M00056977:73 | CH16COP |
| 1744 | 2796 | RTA22200240F.c.13.1.P.Seq | F | M00023331:111 | CH04MAL |
| 1745 | 5753 | RTA22200227F.f.13.1.P.Seq | F | M00006648:15 | CH02COH |
| 1746 | 734256 | RTA22200014F.l.11.2.P.Seq | F | M00057005:43 | CH16COP |
| 1747 | 449580 | RTA22200007F.p.19.1.P.Seq | F | M00056290:58 | CH15CON |
| 1748 | 553705 | RTA22200021F.a.23.2.P.Seq | F | M00054726:68 | CH17COHLV |
| 1749 | 730670 | RTA22200011F.n.02.1.P.Seq | F | M00056638:21 | CH16COP |
| 1750 | 15035 | RTA22200001F.c.07.1.P.Seq | F | M00042544:610 | CH15CON |
| 1751 | 394436 | RTA22200002F.o.12.1.P.Seq | F | M00055552:39 | CH15CON |
| 1752 | 726810 | RTA22200011F.h.22.1.P.Seq | F | M00056592:33 | CH16COP |
| 1753 | 352763 | RTA22200022F.j.12.1.P.Seq | F | M00054949:17 | CH17COHLV |
| 1754 | 3506 | RTA22200240F.i.14.1.P.Seq | F | M00023414:63 | CH04MAL |
| 1755 | 726377 | RTA22200015F.i.14.1.P.Seq | F | M00057100:23 | CH16COP |
| 1756 | 562111 | RTA22200018F.d.10.1.P.Seq | F | M00042460:17 | CH17COHLV |
| 1757 | 404475 | RTA22200010F.m.04.1.P.Seq | F | M00056496:89 | CH16COP |
| 1758 | 13824 | RTA22200234F.m.16.1.P.Seq | F | M00022370:72 | CH03MAH |
| 1759 | 558222 | RTA22200019F.h.07.1.P.Seq | F | M00054502:41 | CH17COHLV |
| 1760 | 2834 | RTA22200224F.p.09.1.P.Seq | F | M00005406:111 | CH02COH |
| 1761 | 453470 | RTA22200001F.a.10.1.P.Seq | F | M00042523:35 | CH15CON |
| 1762 | 558682 | RTA22200015F.o.16.1.P.Seq | F | M00057141:22 | CH16COP |
| 1763 | 641710 | RTA22200016F.j.03.1.P.Seq | F | M00057231:79 | CH16COP |
| 1764 | 640221 | RTA22200013F.k.12.1.P.Seq | F | M00056874:71 | CH16COP |
| 1765 | 559057 | RTA22200016F.n.11.1.P.Seq | F | M00057270:89 | CH16COP |
| 1766 | 551433 | RTA22200025F.m.12.2.P.Seq | F | M00055385:76 | CH17COHLV |
| 1767 | 5729 | RTA22200228F.f.10.1.P.Seq | F | M00006819:25 | CH02COH |
| 1768 | 352763 | RTA22200022F.g.09.1.P.Seq | F | M00054931:19 | CH17COHLV |
| 1769 | 375651 | RTA22200012F.m.08.1.P.Seq | F | M00056757:63 | CH16COP |
| 1770 | 644032 | RTA22200012F.m.04.1.P.Seq | F | M00056756:15 | CH16COP |
| 1771 | 185562 | RTA22200240F.j.15.1.P.Seq | F | M00023428:611 | CH04MAL |
| 1772 | 736349 | RTA22200014F.d.03.2.P.Seq | F | M00056947:69 | CH16COP |
| 1773 | 638870 | RTA22200012F.h.13.1.P.Seq | F | M00056721:67 | CH16COP |
| 1774 | 649719 | RTA22200012F.k.03.1.P.Seq | F | M00056739:48 | CH16COP |
| 1775 | 62016 | RTA22200026F.d.09.1.P.Seq | F | M00055420:74 | CH17COHLV |
| 1776 | 2889 | RTA22200229F.p.17.1.P.Seq | F | M00007084:74 | CH02COH |
| 1777 | 647135 | RTA22200010F.k.08.1.P.Seq | F | M00056480:110 | CH16COP |
| 1778 | 8283 | RTA22200224F.g.10.1.P.Seq | F | M00005309:55 | CH02COH |
| 1779 | 732121 | RTA22200014F.d.06.1.P.Seq | F | M00056949:66 | CH16COP |
| 1780 | 532307 | RTA22200022F.h.09.1.P.Seq | F | M00054937:112 | CH17COHLV |
| 1781 | 6589 | RTA22200226F.n.24.1.P.Seq | F | M00005810:67 | CH02COH |
| 1782 | 554678 | RTA22200010F.b.19.1.P.Seq | F | M00056363:26 | CH16COP |
| 1783 | 450410 | RTA22200021F.a.09.2.P.Seq | F | M00054722:41 | CH17COHLV |
| 1784 | 643924 | RTA22200002F.a.21.1.P.Seq | F | M00055427:61 | CH15CON |
| 1785 | 453719 | RTA22200002F.a.17.1.P.Seq | F | M00055426:86 | CH15CON |
| 1786 | 451811 | RTA22200003F.k.17.1.P.Seq | F | M00055681:72 | CH15CON |
| 1787 | 453059 | RTA22200003F.b.22.1.P.Seq | F | M00055586:62 | CH15CON |
| 1788 | 453457 | RTA22200021F.a.06.2.P.Seq | F | M00054721:611 | CH17COHLV |
| 1789 | 558454 | RTA22200020F.a.20.1.P.Seq | F | M00054572:31 | CH17COHLV |
| 1790 | 417467 | RTA22200025F.c.11.1.P.Seq | F | M00055289:42 | CH17COHLV |
| 1791 | 447850 | RTA22200001F.a.23.1.P.Seq | F | M00042532:68 | CH15CON |
| 1792 | 557948 | RTA22200024F.n.05.1.P.Seq | F | M00055245:19 | CH17COHLV |
| 1793 | 452685 | RTA22200022F.b.14.1.P.Seq | F | M00054877:812 | CH17COHLV |
| 1794 | 446964 | RTA22200019F.e.05.1.P.Seq | F | M00043504:78 | CH17COHLV |
| 1795 | 550318 | RTA22200021F.d.04.2.P.Seq | F | M00054741:310 | CH17COHLV |
| 1796 | 407077 | RTA22200023F.p.13.1.P.Seq | F | M00055134:23 | CH17COHLV |
| 1797 | 650864 | RTA22200007F.o.19.1.P.Seq | F | M00056282:44 | CH15CON |
| 1798 | 644721 | RTA22200003F.p.16.1.P.Seq | F | M00055731:812 | CH15CON |
| 1799 | 485431 | RTA22200013F.l.17.1.P.Seq | F | M00056885:36 | CH16COP |
| 1800 | 651073 | RTA22200007F.f.05.1.P.Seq | F | M00056186:62 | CH15CON |
| 1801 | 725811 | RTA22200012F.o.17.1.P.Seq | F | M00056776:49 | CH16COP |
| 1802 | 645139 | RTA22200005F.k.02.1.P.Seq | F | M00055924:811 | CH15CON |
| 1803 | 185478 | RTA22200248F.j.05.1.P.Seq | F | M00027578:65 | CH04MAL |
| 1804 | 1441 | RTA22200228F.j.04.2.P.Seq | F | M00006859:44 | CH02COH |
| 1805 | 640005 | RTA22200002F.i.16.1.P.Seq | F | M00055511:59 | CH15CON |
| 1806 | 728273 | RTA22200015F.h.04.1.P.Seq | F | M00057091:34 | CH16COP |
| 1807 | 185579 | RTA22200242F.e.07.1.P.Seq | F | M00026996:16 | CH04MAL |
| 1808 | 724473 | RTA22200012F.f.23.1.P.Seq | F | M00056711:31 | CH16COP |
| 1809 | 559674 | RTA22200014F.h.06.1.P.Seq | F | M00056974:64 | CH16COP |
| 1810 | 456026 | RTA22200005F.d.13.1.P.Seq | F | M00055873:53 | CH15CON |
| 1811 | 549320 | RTA22200022F.a.02.1.P.Seq | F | M00054867:37 | CH17COHLV |
| 1812 | 447338 | RTA22200001F.k.04.1.P.Seq | F | M00042746:29 | CH15CON |
| 1813 | 560700 | RTA22200002F.b.09.1.P.Seq | F | M00055430.82 | CH15CON |
| 1814 | 3070 | RTA22200021F.g.19.3.P.Seq | F | M00054769:67 | CH17COHLV |
| 1815 | 3070 | RTA22200002F.f.03.1.P.Seq | F | M00055463:810 | CH15CON |
| 1816 | 380477 | RTA22200014F.j.09.2.P.Seq | F | M00056993:26 | CH16COP |
| 1817 | 735040 | RTA22200015F.b.05.1.P.Seq | F | M00057047:32 | CH16COP |
| 1818 | 378525 | RTA22200009F.j.16.2.P.Seq | F | M00042826:33 | CH16COP |
| 1819 | 284586 | RTA22200009F.c.23.2.P.Seq | F | M00042756:21 | CH16COP |
| 1820 | 640276 | RTA22200014F.e.15.2.P.Seq | F | M00056956:61 | CH16COP |
| 1821 | 3344 | RTA22200230F.j.07.1.P.Seq | F | M00007178:43 | CH02COH |
| 1822 | 555830 | RTA22200005F.b.23.1.P.Seq | F | M00055861:43 | CH15CON |
| 1823 | 726307 | RTA22200011F.i.10.1.P.Seq | F | M00056595:62 | CH16COP |
| 1824 | 416 | RTA22200011F.m.06.1.P.Seq | F | M00056631:75 | CH16COP |
| 1825 | 2543 | RTA22200225F.o.10.1.P.Seq | F | M00005545:18 | CH02COH |
| 1826 | 639352 | RTA22200001F.n.09.1.P.Seq | F | M00042908:71 | CH15CON |
| 1827 | 453592 | RTA22200009F.l.04.1.P.Seq | F | M00042842:31 | CH16COP |
| 1828 | 450633 | RTA22200006F.n.19.2.P.Seq | F | M00056107:56 | CH15CON |
| 1829 | 448383 | RTA22200009F.l.15.2.P.Seq | F | M00042844:312 | CH16COP |
| 1830 | 648719 | RTA22200008F.a.08.1.P.Seq | F | M00056293:65 | CH15CON |
| 1831 | 730655 | RTA22200012F.h.16.1.P.Seq | F | M00056722:71 | CH16COP |
| 1832 | 141185 | RTA22200007F.j.21.2.P.Seq | F | M00056228:17 | CH15CON |
| 1833 | 640498 | RTA22200003F.m.07.1.P.Seq | F | M00055703:28 | CH15CON |
| 1834 | 9029 | RTA22200222F.l.08.1.P.Seq | F | M00004087:211 | CH01COH |
| 1835 | 559674 | RTA22200014F.h.06.2.P.Seq | F | M00056974:64 | CH16COP |
| 1836 | 555734 | RTA22200021F.n.09.2.P.Seq | F | M00054844:66 | CH17COHLV |
| 1837 | 1943 | RTA22200017F.d.22.1.P.Seq | F | M00057333:69 | CH16COP |
| 1838 | 648320 | RTA22200004F.d.05.1.P.Seq | F | M00055761:33 | CH15CON |
| 1839 | 558098 | RTA22200024F.p.10.1.P.Seq | F | M00055260:15 | CH17COHLV |
| 1840 | 468672 | RTA22200004F.h.23.1.P.Seq | F | M00055794:26 | CH15CON |
| 1841 | 456596 | RTA22200022F.f.19.1.P.Seq | F | M00054926:61 | CH17COHLV |
| 1842 | 649722 | RTA22200012F.n.07.1.P.Seq | F | M00056765:410 | CH16COP |
| 1843 | 550708 | RTA22200022F.o.19.1.P.Seq | F | M00054996:211 | CH17COHLV |
| 1844 | 643931 | RTA22200005F.n.14.1.P.Seq | F | M00055966:13 | CH15CON |
| 1845 | 726927 | RTA22200011F.j.08.1.P.Seq | F | M00056603:41 | CH16COP |
| 1846 | 459012 | RTA22200007F.b.13.1.P.Seq | F | M00056149:21 | CH15CON |
| 1847 | 397773 | RTA22200003F.c.13.1.P.Seq | F | M00055592:15 | CH15CON |
| 1848 | 450004 | RTA22200011F.f.04.1.P.Seq | F | M00056566:81 | CH16COP |
| 1849 | 649732 | RTA22200007F.o.23.1.P.Seq | F | M00056282:89 | CH15CON |
| 1850 | 553955 | RTA22200021F.f.08.3.P.Seq | F | M00054755:86 | CH17COHLV |
| 1851 | 646309 | RTA22200012F.h.04.1.P.Seq | F | M00056718:21 | CH16COP |
| 1852 | 402727 | RTA22200024F.m.16.1.P.Seq | F | M00055243:64 | CH17COHLV |
| 1853 | 468736 | RTA22200024F.m.17.1.P.Seq | F | M00055243:71 | CH17COHLV |
| 1854 | 650422 | RTA22200007F.a.16.1.P.Seq | F | M00056141:45 | CH15CON |
| 1855 | 730533 | RTA22200013F.b.15.1.P.Seq | F | M00056808:212 | CH16COP |
| 1856 | 726307 | RTA22200015F.a.17.1.P.Seq | F | M00057044:66 | CH16COP |
| 1857 | 450311 | RTA22200005F.j.22.1.P.Seq | F | M00055922:32 | CH15CON |
| 1858 | 450940 | RTA22200001F.j.15.1.P.Seq | F | M00042742:83 | CH15CON |
| 1859 | 726786 | RTA22200015F.f.06.1.P.Seq | F | M00057077:42 | CH16COP |
| 1860 | 7634 | RTA22200235F.b.19.1.P.Seq | F | M00022420:83 | CH03MAH |
| 1861 | 230995 | RTA22200016F.e.14.1.P.Seq | F | M00057196:64 | CH16COP |
| 1862 | 374770 | RTA22200003F.n.21.1.P.Seq | F | M00055720:16 | CH15CON |
| 1863 | 9275 | RTA22200249F.g.11.1.P.Seq | F | M00027665:41 | CH04MAL |
| 1864 | 553860 | RTA22200023F.k.02.1.P.Seq | F | M00055077:611 | CH17COHLV |
| 1865 | 452010 | RTA22200001F.m.07.1.P.Seq | F | M00042900:37 | CH15CON |
| 1866 | 649560 | RTA22200015F.j.16.1.P.Seq | F | M00057106:52 | CH16COP |
| 1867 | 452704 | RTA22200008F.b.18.1.P.Seq | F | M00056307:812 | CH15CON |
| 1868 | 447594 | RTA22200007F.j.10.1.P.Seq | F | M00056225:79 | CH15CON |
| 1869 | 555444 | RTA22200015F.n.10.1.P.Seq | F | M00057130:811 | CH16COP |
| 1870 | 736556 | RTA22200017F.c.20.1.P.Seq | F | M00057318:13 | CH16COP |
| 1871 | 5289 | RTA22200235F.l.12.1.P.Seq | F | M00022533:56 | CH03MAH |
| 1872 | 732121 | RTA22200015F.c.11.1.P.Seq | F | M00057059:66 | CH16COP |
| 1873 | 452567 | RTA22200009F.f.12.1.P.Seq | F | M00042783:13 | CH16COP |
| 1874 | 551634 | RTA22200002F.n.04.1.P.Seq | F | M00055544:54 | CH15CON |
| 1875 | 644099 | RTA22200005F.g.07.1.P.Seq | F | M00055887:67 | CH15CON |
| 1876 | 726788 | RTA22200012F.h.08.1.P.Seq | F | M00056719:19 | CH16COP |
| 1877 | 638802 | RTA22200012F.a.02.1.P.Seq | F | M00056662:63 | CH16C0P |
| 1878 | 646283 | RTA22200002F.o.18.1.P.Seq | F | M00055553:82 | CH15CON |
| 1879 | 8403 | RTA22200238F.n.03.1.P.Seq | F | M00022998:38 | CH03MAH |
| 1880 | 2224 | RTA22200233F.g.23.1.P.Seq | F | M00008079:33 | CH03MAH |
| 1881 | 650053 | RTA22200008F.g.02.1.P.Seq | F | M00056459:47 | CH15CON |
| 1882 | 380477 | RTA22200008F.e.20.1.P.Seq | F | M00056437:87 | CH15CON |
| 1883 | 450867 | RTA22200019F.a.09.1.P.Seq | F | M00043386:16 | CH17COHLV |
| 1884 | 456764 | RTA22200003F.n.22.1.P.Seq | F | M00055720:11 | CH15CON |
| 1885 | 641373 | RTA22200003F.b.11.1.P.Seq | F | M00055583:24 | CH15CON |
| 1886 | 555882 | RTA22200004F.m.12.1.P.Seq | F | M00055818:510 | CH15CON |
| 1887 | 644046 | RTA22200008F.b.04.1.P.Seq | F | M00056302:612 | CH15CON |
| 1888 | 447250 | RTA22200009F.d.15.2.P.Seq | F | M00042760:37 | CH16COP |
| 1889 | 456596 | RTA22200022F.o.14.1.P.Seq | F | M00054995:310 | CH17COHLV |
| 1890 | 2218 | RTA22200225F.d.15.1.P.Seq | F | M00005447:41 | CH02COH |
| 1891 | 446450 | RTA22200001F.h.16.1.P.Seq | F | M00042718:33 | CH15CON |
| 1892 | 640889 | RTA22200003F.l.12.1.P.Seq | F | M00055691:57 | CH15C0N |
| 1893 | 530774 | RTA22200025F.n.12.2.P.Seq | F | M00055391:78 | CH17COHLV |
| 1894 | 649062 | RTA22200002F.j.14.1.P.Seq | F | M00055519:87 | CH15CON |
| 1895 | 12808 | RTA22200233F.m.15.1.P.Seq | F | M00021649:512 | CH03MAH |
| 1896 | 468672 | RTA22200001F.g.11.1.P.Seq | F | M00042704:64 | CH15CON |
| 1897 | 650773 | RTA22200008F.d.12.1.P.Seq | F | M00056324:42 | CH15CON |
| 1898 | 732237 | RTA22200014F.i.12.2.P.Seq | F | M00056994:67 | CH16COP |
| 1899 | 650773 | RTA22200001F.o.15.1.P.Seq | F | M00054798:61 | CH15CON |
| 1900 | 550216 | RTA22200021F.b.09.2.P.Seq | F | M00054727:86 | CH17COHLV |
| 1901 | 639189 | RTA22200002F.n.20.1.P.Seq | F | M00055547:55 | CH15CON |
| 1902 | 3447 | RTA22200227F.l.17.1.P.Seq | F | M00006719:87 | CH02COH |
| 1903 | 2012 | RTA22200248F.c.03.1.P.Seq | F | M00027524:211 | CH04MAL |
| 1904 | 642876 | RTA22200005F.b.13.1.P.Seq | F | M00055856:64 | CH15CON |
| 1905 | 449690 | RTA22200009F.j.10.2.P.Seq | F | M00042823:74 | CH16COP |
| 1906 | 451208 | RTA22200004F.k.07.1.P.Seq | F | M00055804:63 | CH15CON |
| 1907 | 725811 | RTA22200011F.k.18.1.P.Seq | F | M00056616:38 | CH16COP |
| 1908 | 1256 | RTA22200232F.b.11.1.P.Seq | F | M00021869:41 | CH03MAH |
| 1909 | 446599 | RTA22200004F.f.18.1.P.Seq | F | M00055780:78 | CH15CON |
| 1910 | 446537 | RTA22200001F.c.05.1.P.Seq | F | M00042544:42 | CH15CON |
| 1911 | 726281 | RTA22200010F.l.02.1.P.Seq | F | M00056484:72 | CH16COP |
| 1912 | 11286 | RTA22200226F.n.17.1.P.Seq | F | M00005802:810 | CH02COH |
| 1913 | 556082 | RTA22200022F.p.11.1.P.Seq | F | M00055005:811 | CH17COHLV |
| 1914 | 97507 | RTA22200005F.a.21.1.P.Seq | F | M00055851:711 | CH17COHLV |
| 1915 | 535955 | RTA22200022F.b.11.1.P.Seq | F | M00054876:73 | CH17COHLV |
| 1916 | 728251 | RTA22200011F.i.08.1.P.Seq | F | M00056595:37 | CH16COP |
| 1917 | 733849 | RTA22200015F.g.08.1.P.Seq | F | M00057085:13 | CH16COP |
| 1918 | 447574 | RTA22200014F.k.20.2.P.Seq | F | M00057002:25 | CH16COP |
| 1919 | 7607 | RTA22200229F.g.17.1.P.Seq | F | M00006976:45 | CH02COH |
| 1920 | 644032 | RTA22200010F.i.21.1.P.Seq | F | M00056425:23 | CH16COP |
| 1921 | 454087 | RTA22200012F.f.13.1.P.Seq | F | M00056709:15 | CH16COP |
| 1922 | 412364 | RTA22200007F.p.02.1.P.Seq | F | M00056283:33 | CH15CON |
| 1923 | 535208 | RTA22200002F.m.04.1.P.Seq | F | M00055534:81 | CH15CON |
| 1924 | 644609 | RTA22200002F.m.21.1.P.Seq | F | M00055542:66 | CH15CON |
| 1925 | 645073 | RTA22200004F.o.14.1.P.Seq | F | M00055830:710 | CH15CON |
| 1926 | 417467 | RTA22200012F.g.08.1.P.Seq | F | M00056712:26 | CH16COP |
| 1927 | 554188 | RTA22200004F.n.02.1.P.Seq | F | M00055821:712 | CH15CON |
| 1928 | 647185 | RTA22200005F.n.02.1.P.Seq | F | M00055958:62 | CH15CON |
| 1929 | 736679 | RTA22200012F.a.20.1.P.Seq | F | M00056666:38 | CH16COP |
| 1930 | 553547 | RTA22200022F.j.02.1.P.Seq | F | M00054947:712 | CH17COHLV |
| 1931 | 641524 | RTA22200016F.c.06.1.P.Seq | F | M00057170:83 | CH16COP |
| 1932 | 649717 | RTA22200003F.f.02.1.P.Seq | F | M00055633:12 | CH15CON |
| 1933 | 451041 | RTA22200018F.a.16.1.P.Seq | F | M00042355:31 | CH17COHLV |
| 1934 | 3483 | RTA22200225F.e.24.1.P.Seq | F | M00005459:21 | CH02COH |
| 1935 | 500959 | RTA22200008F.c.24.1.P.Seq | F | M00056323:810 | CH15CON |
| 1936 | 500959 | RTA22200008F.d.01.1.P.Seq | F | M00056323:810 | CH15CON |
| 1937 | 697 | RTA22200233F.b.10.1.P.Seq | F | M00008020:33 | CH03MAH |
| 1938 | 736955 | RTA22200013F.f.22.1.P.Seq | F | M00056840:89 | CH16COP |
| 1939 | 554742 | RTA22200004F.l.14.1.P.Seq | F | M00055811:312 | CH15CON |
| 1940 | 642973 | RTA22200015F.n.14.1.P.Seq | F | M00057132:68 | CH16COP |
| 1941 | 449437 | RTA22200008F.b.13.1.P.Seq | F | M00056304:711 | CH15CON |
| 1942 | 467991 | RTA22200002F.j.11.1.P.Seq | F | M00055517:43 | CH15CON |
| 1943 | 650204 | RTA22200004F.n.13.1.P.Seq | F | M00055825:53 | CH15CON |
| 1944 | 640618 | RTA22200004F.p.17.1.P.Seq | F | M00055840:46 | CH15CON |
| 1945 | 452366 | RTA22200002F.j.05.1.P.Seq | F | M00055514:85 | CH15CON |
| 1946 | 640276 | RTA22200012F.p.05.1.P.Seq | F | M00056780:69 | CH16COP |
| 1947 | 554101 | RTA22200012F.f.24.1.P.Seq | F | M00056711:65 | CH16COP |
| 1948 | 185432 | RTA22200249F.p.01.1.P.Seq | F | M00027742:21 | CH04MAL |
| 1949 | 455598 | RTA22200022F.m.06.1.P.Seq | F | M00054970:73 | CH17COHLV |
| 1950 | 649354 | RTA22200001F.o.05.1.P.Seq | F | M00054792:59 | CH15CON |
| 1951 | 4408 | RTA22200242F.j.13.1.P.Seq | F | M00027034:49 | CH04MAL |
| 1952 | 452366 | RTA22200006F.o.03.2.P.Seq | F | M00056110:49 | CH15CON |
| 1953 | 452366 | RTA22200001F.e.02.1.P.Seq | F | M00042563:79 | CH15CON |
| 1954 | 727331 | RTA22200016F.a.19.1.P.Seq | F | M00057161:59 | CH16COP |
| 1955 | 644853 | RTA22200008F.a.05.1.P.Seq | F | M00056293:58 | CH15CON |
| 1956 | 554079 | RTA22200014F.k.21.2.P.Seq | F | M00057002:26 | CH16COP |
| 1957 | 556245 | RTA22200023F.l.05.1.P.Seq | F | M00055088:112 | CH17COHLV |
| 1958 | 557388 | RTA22200012F.p.13.1.P.Seq | F | M00056783:711 | CH16COP |
| 1959 | 449468 | RTA22200012F.l.11.1.P.Seq | F | M00056752:51 | CH16COP |
| 1960 | 556245 | RTA22200023F.o.12.1.P.Seq | F | M00055125:56 | CH17COHLV |
| 1961 | 455327 | RTA22200013F.b.03.1.P.Seq | F | M00056804:56 | CH16COP |
| 1962 | 546632 | RTA22200015F.a.10.1.P.Seq | F | M00057041:211 | CH16COP |
| 1963 | 558762 | RTA22200022F.a.03.1.P.Seq | F | M00054867:22 | CH17COHLV |
| 1964 | 550818 | RTA22200020F.d.10.1.P.Seq | F | M00054590:72 | CH17COHLV |
| 1965 | 554079 | RTA22200021F.p.09.2.P.Seq | F | M00054862:27 | CH17COHLV |
| 1966 | 452430 | RTA22200016F.e.07.1.P.Seq | F | M00057192:52 | CH16COP |
| 1967 | 452430 | RTA22200008F.e.11.1.P.Seq | F | M00056342:611 | CH15CON |
| 1968 | 556082 | RTA22200019F.i.02.1.P.Seq | F | M00064507:311 | CH17COHLV |
| 1969 | 514418 | RTA22200023F.e.23.1.P.Seq | F | M00055046:511 | CH17COHLV |
| 1970 | 426895 | RTA22200021F.p.14.2.P.Seq | F | M00054863:73 | CH17COHLV |
| 1971 | 560803 | RTA22200025F.c.05.1.P.Seq | F | M00055283:65 | CH17COHLV |
| 1972 | 447737 | RTA22200018F.c.11.1.P.Seq | F | M00042453:51 | CH17COHLV |
| 1973 | 373432 | RTA22200018F.l.18.1.P.Seq | F | M00043350:89 | CH17COHLV |
| 1974 | 779 | RTA22200250F.f.02.1.P.Seq | F | M00027820:32 | CH04MAL |
| 1975 | 455327 | RTA22200015F.m.05.1.P.Seq | F | M00057124:410 | CH16COP |
| 1976 | 554742 | RTA22200004F.k.16.1.P.Seq | F | M00055806:812 | CH15CON |
| 1977 | 455327 | RTA22200022F.g.14.1.P.Seq | F | M00054933:47 | CH17COHLV |
| 1978 | 11043 | RTA22200228F.j.10.2.P.Seq | F | M00006861:18 | CH02COH |
| 1979 | 727447 | RTA22200012F.c.14.1.P.Seq | F | M00056684:15 | CH16COP |
| 1980 | 552905 | RTA22200011F.i.16.1.P.Seq | F | M00056597:67 | CH16COP |
| 1981 | 446900 | RTA22200001F.g.23.1.P.Seq | F | M00042711:711 | CH15CON |
| 1982 | 644190 | RTA22200005F.j.02.1.P.Seq | F | M00055913:45 | CH15CON |
| 1983 | 455327 | RTA22200021F.c.18.2.P.Seq | F | M00054739:73 | CH17COHLV |
| 1984 | 422375 | RTA22200020F.g.01.1.P.Seq | F | M00054613:49 | CH17COHLV |
| 1985 | 422375 | RTA22200020F.f.24.1.P.Seq | F | M00054613:49 | CH17COHLV |
| 1986 | 530774 | RTA22200025F.p.22.2.P.Seq | F | M00055405:49 | CH17COHLV |
| 1987 | 554101 | RTA22200012F.g.10.1.P.Seq | F | M00056711:65 | CH16COP |
| 1988 | 5268 | RTA22200226F.g.10.1.P.Seq | F | M00005650:44 | CH02COH |
| 1989 | 642461 | RTA22200012F.g.18.1.P.Seq | F | M00056715:71 | CH16COP |
| 1990 | 770 | RTA22200003F.a.18.1.P.Seq | F | M00055575:48 | CH15CON |
| 1991 | 3837 | RTA22200231F.b.24.1.P.Seq | F | M00007936:29 | CH03MAH |
| 1992 | 561382 | RTA22200022F.d.20.1.P.Seq | F | M00054899:71 | CH17COHLV |
| 1993 | 4408 | RTA22200248F.e.13.1.P.Seq | F | M00027543:29 | CH04MAL |
| 1994 | 5686 | RTA22200234F.j.21.1.P.Seq | F | M00022279:84 | CH03MAH |
| 1995 | 374609 | RTA22200013F.e.23.1.P.Seq | F | M00056833:31 | CH16C0P |
| 1996 | 734793 | RTA22200012F.h.06.1.P.Seq | F | M00056719:46 | CH16COP |
| 1997 | 452430 | RTA2220O014F.f.18.2.P.Seq | F | M00056964:12 | CH16COP |
| 1998 | 450940 | RTA22200020F.n.11.1.P.Seq | F | M00054684:812 | CH17COHLV |
| 1999 | 460445 | RTA22200024F.c.23.1.P.Seq | F | M00055162:66 | CH17COHLV |
| 2000 | 549041 | RTA22200020F.i.01.1.P.Seq | F | M00054636:23 | CH17COHLV |
| 2001 | 555276 | RTA22200018F.g.13.1.P.Seq | F | M00043313:77 | CH17COHLV |
| 2002 | 426895 | RTA22200015F.l.18.1.P.Seq | F | M00057120:512 | CH16COP |
| 2003 | 1833 | RTA22200224F.f.16.1.P.Seq | F | M00005293:46 | CH02COH |
| 2004 | 446450 | RTA22200009F.h.08.1.P.Seq | F | M00042805:56 | CH16COP |
| 2005 | 650517 | RTA22200007F.l.18.2.P.Seq | F | M00056248:19 | CH15CON |
| 2006 | 554785 | RTA22200026F.f.14.1.P.Seq | F | M00055479:312 | CH17COHLV |
| 2007 | 607430 | RTA22200002F.i.06.1.P.Seq | F | M00055508:53 | CH15CON |
| 2008 | 446673 | RTA22200012F.a.18.1.P.Seq | F | M00056665:55 | CH16COP |
| 2009 | 734685 | RTA22200014F.i.17.2.P.Seq | F | M00056986:71 | CH16COP |
| 2010 | 11630 | RTA22200248F.h.02.1.P.Seq | F | M00027562:32 | CH04MAL |
| 2011 | 2930 | RTA22200228F.c.13.1.P.Seq | F | M00006795:47 | CH02COH |
| 2012 | 44424 | RTA22200006F.f.07.2.P.Seq | F | M00056038:64 | CH15CON |
| 2013 | 452052 | RTA22200001F.e.19.1.P.Seq | F | M00042574:88 | CH15CON |
| 2014 | 449356 | RTA22200009F.j.14.2.P.Seq | F | M00042826:35 | CH16COP |
| 2015 | 726225 | RTA22200010F.d.04.1.P.Seq | F | M00056372:310 | CH16COP |
| 2016 | 453708 | RTA22200022F.j.14.1.P.Seq | F | M00054950:76 | CH17COHLV |
| 2017 | 447858 | RTA22200003F.n.20.1.P.Seq | F | M00055720:79 | CH15CON |
| 2018 | 451613 | RTA22200018F.e.18.1.P.Seq | F | M00043301:89 | CH17COHLV |
| 2019 | 650337 | RTA22200008F.c.18.1.P.Seq | F | M00056319:71 | CH15CON |
| 2020 | 62016 | RTA22200019F.k.10.1.P.Seq | F | M00054522:811 | CH17COHLV |
| 2021 | 447250 | RTA22200014F.f.02.2.P.Seq | F | M00056960:35 | CH16COP |
| 2022 | 3837 | RTA22200231F.c.01.1.P.Seq | F | M00007936:29 | CH03MAH |
| 2023 | 640614 | RTA22200017F.e.22.1.P.Seq | F | M00057340:312 | CH16COP |
| 2024 | 729531 | RTA22200013F.g.24.1.P.Seq | F | M00056850:511 | CH16COP |
| 2025 | 729531 | RTA22200013F.h.01.1.P.Seq | F | M00056850:511 | CH16COP |
| 2026 | 647952 | RTA22200004F.j.07.1.P.Seq | F | M00055800:38 | CH15CON |
| 2027 | 446913 | RTA22200004F.k.19.1.P.Seq | F | M00055806:67 | CH15CON |
| 2028 | 2675 | RTA22200233F.a.20.1.P.Seq | F | M00008015:68 | CH03MAH |
| 2029 | 643481 | RTA22200003F.i.13.1.P.Seq | F | M00055664:18 | CH15CON |
| 2030 | 1345 | RTA22200012F.l.22.1.P.Seq | F | M00056754:84 | CH16COP |
| 2031 | 26 | RTA22200231F.a.03.1.P.Seq | F | M00007926:15 | CH03MAH |
| 2032 | 945 | RTA22200230F.h.02.1.P.Seq | F | M00007156:56 | CH02COH |
| 2033 | 449169 | RTA22200009F.b.09.2.P.Seq | F | M00042461:110 | CH16COP |
| 2034 | 394193 | RTA22200007F.h.10.1.P.Seq | F | M00056209:612 | CH15CON |
| 2035 | 452212 | RTA22200006F.h.13.2.P.Seq | F | M00056055:21 | CH15CON |
| 2036 | 394193 | RTA22200007F.i.15.2.P.Seq | F | M00056220:42 | CH15CON |
| 2037 | 1310 | RTA22200235F.o.10.2.P.Seq | F | M00022561:16 | CH03MAH |
| 2038 | 734094 | RTA22200016F.k.07.1.P.Seq | F | M00057241:24 | CH16COP |
| 2039 | 646579 | RTA22200002F.h.08.1.P.Seq | F | M00055498:89 | CH15CON |
| 2040 | 4471 | RTA22200233F.j.22.1.P.Seq | F | M00008098:81 | CH03MAH |
| 2041 | 729173 | RTA22200016F.e.13.1.P.Seq | F | M00057196:53 | CH16COP |
| 2042 | 450323 | RTA22200009F.i.11.2.P.Seq | F | M00042814:211 | CH16COP |
| 2043 | 4652 | RTA22200241F.n.17.1.P.Seq | F | M00026950:612 | CH04MAL |
| 2044 | 553316 | RTA00022201F.a.10.2.P.Seq | F | M00054722:38 | CH17COHLV |
| 2045 | 642604 | RTA22200016F.j.10.1.P.Seq | F | M00057233:67 | CH16COP |
| 2046 | 553316 | RTA22200002F.i.02.1.P.Seq | F | M00055504:48 | CH15CON |
| 2047 | 4097 | RTA22200249F.f.14.1.P.Seq | F | M00027652:73 | CH04MAL |
| 2048 | 6818 | RTA22200006F.f.12.2.P.Seq | F | M00056042:11 | CH15CON |
| 2049 | 395341 | RTA22200024F.c.22.1.P.Seq | F | M00055161:111 | CH17COHLV |
| 2050 | 649143 | RTA22200007F.o.20.1.P.Seq | F | M00056282:31 | CH15CON |
| 2051 | 649143 | RTA22200007F.o.18.1.P.Seq | F | M00056282:611 | CH15CON |
| 2052 | 648310 | RTA22200007F.e.16.1.P.Seq | F | M00056184:61 | CH15CON |
| 2053 | 447574 | RTA22200014F.k.20.1.P.Seq | F | M00057002:25 | CH16COP |
| 2054 | 648931 | RTA22200004F.b.12.1.P.Seq | F | M00055744:68 | CH15CON |
| 2055 | 6878 | RTA22200231F.a.01.1.P.Seq | F | M00007926:17 | CH03MAH |
| 2056 | 452238 | RTA22200009F.d.09.2.P.Seq | F | M00042758:812 | CH16COP |
| 2057 | 1870 | RTA22200238F.g.09.1.P.Seq | F | M00022911:71 | CH05MAH |
| 2058 | 559259 | RTA22200002F.n.12.1.P.Seq | F | M00055545:812 | CH15CON |
| 2059 | 453457 | RTA22200004F.a.06.1.P.Seq | F | M00055735:37 | CH15CON |
| 2060 | 8868 | RTA22200233F.l.13.1.P.Seq | F | M00021626:64 | CH03MAH |
| 2061 | 453059 | RTA22200001F.b.21.1.P.Seq | F | M00042540:63 | CH15CON |
| 2062 | 236368 | RTA22200008F.d.09.1.P.Seq | F | M00056323:17 | CH15CON |
| 2063 | 453059 | RTA22200001F.b.18.1.P.Seq | F | M00042539:55 | CH15CON |
| 2064 | 549979 | RTA22200007F.c.06.1.P.Seq | F | M00056153:41 | CH15CON |
| 2065 | 515631 | RTA22200014F.j.22.2.P.Seq | F | M00056996:112 | CH16COP |
| 2066 | 2235 | RTA22200234F.f.22.1.P.Seq | F | M00022236:12 | CH03MAH |
| 2067 | 448193 | RTA22200016F.n.15.1.P.Seq | F | M00057271:51 | CH16COP |
| 2068 | 530774 | RTA22200010F.c.12.1.P.Seq | F | M00056368:64 | CH16COP |
| 2069 | 650204 | RTA22200002F.d.14.1.P.Seq | F | M00055450:28 | CH15CON |
| 2070 | 644240 | RTA22200005F.j.19.1.P.Seq | F | M00055921:53 | CH15CON |
| 2071 | 552614 | RTA22200022F.l.11.1.P.Seq | F | M00054965:82 | CH17COHLV |
| 2072 | 727331 | RTA22200011F.b.04.1.P.Seq | F | M00056532:76 | CH16COP |
| 2073 | 185457 | RTA22200244F.n.05.1.P.Seq | F | M00027220:212 | CH04MAL |
| 2074 | 454531 | RTA22200022F.a.11.1.P.Seq | F | M00054869:41 | CH17COHLV |
| 2075 | 643485 | RTA22200006F.g.19.2.P.Seq | F | M00056050:13 | CH15CON |
| 2076 | 733669 | RTA22200013F.m.22.1.P.Seq | F | M00056895:17 | CH16COP |
| 2077 | 452344 | RTA22200016F.b.19.1.P.Seq | F | M00057167:27 | CH16COP |
| 2078 | 63602 | RTA22200002F.b.11.1.P.Seq | F | M00055431:59 | CH15CON |
| 2079 | 454155 | RTA22200004F.j.13.1.P.Seq | F | M00055802:48 | CH15CON |
| 2080 | 549903 | RTA22200013F.b.22.1.P.Seq | F | M00056811:34 | CH16COP |
| 2081 | 515631 | RTA22200014F.j.22.1.P.Seq | F | M00056996:112 | CH16COP |
| 2082 | 6878 | RTA22200248F.j.20.1.P.Seq | F | M00027580:510 | CH04MAL |
| 2083 | 2977 | RTA22200231F.l.18.1.P.Seq | F | M00007983:86 | CH03MAH |
| 2084 | 553823 | RTA22200006F.i.02.2.P.Seq | F | M00056060:210 | CH15CON |
| 2085 | 3070 | RTA22200026F.a.07.1.P.Seq | F | M00055405:77 | CH17COHLV |
| 2086 | 728884 | RTA22200012F.k.06.1.P.Seq | F | M00056740:25 | CH16COP |
| 2087 | 8166 | RTA22200009F.c.03.2.P.Seq | F | M00042469:84 | CH16COP |
| 2088 | 644190 | RTA22200010F.b.18.1.P.Seq | F | M00056362:75 | CH16COP |
| 2089 | 733669 | RTA22200015F.l.05.1.P.Seq | F | M00057117:711 | CH16COP |
| 2090 | 728273 | RTA22200011F.i.07.1.P.Seq | F | M00056595:12 | CH16COP |
| 2091 | 406499 | RTA22200004F.a.13.1.P.Seq | F | M00055736:77 | CH15CON |
| 2092 | 557720 | RTA22200022F.k.07.1.P.Seq | F | M00054954:61 | CH17COHLV |
| 2093 | 732050 | RTA22200015F.d.10.1.P.Seq | F | M00057065:44 | CH16COP |
| 2094 | 450867 | RTA22200020F.j.18.1.P.Seq | F | M00054648:810 | CH17COHLV |
| 2095 | 650297 | RTA22200016F.a.01.1.P.Seq | F | M00057151:24 | CH16COP |
| 2096 | 448064 | RTA22200009F.f.08.1.P.Seq | F | M00042781:16 | CH16COP |
| 2097 | 452530 | RTA22200018F.p.03.1.P.Seq | F | M00043374:26 | CH17COHLV |
| 2098 | 7592 | RTA22200225F.m.10.1.P.Seq | F | M00005515:84 | CH02COH |
| 2099 | 733669 | RTA22200016F.b.13.1.P.Seq | F | M00057165:512 | CH16COP |
| 2100 | 11028 | RTA22200228F.d.13.1.P.Seq | F | M00006806:39 | CH02COH |
| 2101 | 1013 | RTA22200001F.h.22.1.P.Seq | F | M00042720:710 | CH15CON |
| 2102 | 549265 | RTA22200002F.h.03.1.P.Seq | F | M00055496:39 | CH15CON |
| 2103 | 376600 | RTA22200013F.p.08.1.P.Seq | F | M00056911:62 | CH16COP |
| 2104 | 643804 | RTA22200004F.p.14.1.P.Seq | F | M00055839:110 | CH15CON |
| 2105 | 454927 | RTA22200026F.d.08.1.P.Seq | F | M00055420:610 | CH17COHLV |
| 2106 | 446528 | RTA22200006F.i.06.2.P.Seq | F | M00056061:66 | CH15CON |
| 2107 | 2218 | RTA22200226F.n.14.1.P.Seq | F | M00005800:411 | CH02COH |
| 2108 | 452704 | RTA22200004F.a.01.1.P.Seq | F | M00055733:711 | CH15CON |
| 2109 | 84895 | RTA22200014F.m.13.2.P.Seq | F | M00057013:41 | CH16COP |
| 2110 | 157629 | RTA22200242F.c.11.1.P.Seq | F | M00026984:410 | CH04MAL |
| 2111 | 2930 | RTA22200229F.h.22.1.P.Seq | F | M00006990:52 | CH02COH |
| 2112 | 7037 | RTA22200230F.k.08.1.P.Seq | F | M00007194:24 | CH02COH |
| 2113 | 559806 | RTA22200002F.i.04.1.P.Seq | F | M00055505:110 | CH15CON |
| 2114 | 452076 | RTA22200004F.g.03.1.P.Seq | F | M00055783:311 | CH15CON |
| 2115 | 454869 | RTA22200004F.e.14.1.P.Seq | F | M00055771:44 | CH15CON |
| 2116 | 559674 | RTA22200023F.j.05.1.P.Seq | F | M00055071:12 | CH17COHLV |
| 2117 | 2235 | RTA22200232F.n.21.1.P.Seq | F | M00022149:31 | CH03MAH |
| 2118 | 7545 | RTA22200222F.k.06.1.P.Seq | F | M00004054:75 | CH01COH |
| 2119 | 729173 | RTA22200016F.n.22.1.P.Seq | F | M00057272:11 | CH16COP |
| 2120 | 650448 | RTA22200004F.m.09.1.P.Seq | F | M00055818:612 | CH15CON |
| 2121 | 172013 | RTA22200002F.j.16.1.P.Seq | F | M00055520:16 | CH15CON |
| 2122 | 651088 | RTA22200014F.c.15.1.P.Seq | F | M00056945:211 | CH16COP |
| 2123 | 651088 | RTA22200014F.c.15.2.P.Seq | F | M00056945:211 | CH16COP |
| 2124 | 726810 | RTA22200015F.o.06.1.P.Seq | F | M00057136:61 | CH16COP |
| 2125 | 406499 | RTA22200004F.n.23.1.P.Seq | F | M00055827:12 | CH15CON |
| 2126 | 556325 | RTA22200023F.l.03.1.P.Seq | F | M00055087:110 | CH17COHLV1 |
| 2127 | 644836 | RTA22200004F.i.13.1.P.Seq | F | M00055797:48 | CH15CON |
| 2128 | 649062 | RTA22200007F.d.04.1.P.Seq | F | M00056162:59 | CH15CON |
| 2129 | 454776 | RTA22200011F.d.07.1.P.Seq | F | M00056551:62 | CH16COP |
| 2130 | 377579 | RTA22200025F.f.17.1.P.Seq | F | M00055332:711 | CH17COHLV |
| 2131 | 728131 | RTA22200014F.o.08.1.P.Seq | F | M00057024:75 | CH16COP |
| 2132 | 475203 | RTA22200016F.a.05.1.P.Seq | F | M00057154:24 | CH16COP |
| 2133 | 727314 | RTA22200011F.p.15.1.P.Seq | F | M00056659:78 | CH16COP |
| 2134 | 552025 | RTA22200019F.k.15.1.P.Seq | F | M00054523:110 | CH17COHLV |
| 2135 | 561382 | RTA22200004F.d.11.1.P.Seq | F | M00055765:25 | CH15CON |
| 2136 | 732579 | RTA22200017F.a.22.1.P.Seq | F | M00057300:62 | CH16COP |
| 2137 | 167 | RTA22200012F.o.22.1.P.Seq | F | M00056779:512 | CH16COP |
| 2138 | 185585 | RTA22200241F.l.21.1.P.Seq | F | M00026937:28 | CH04MAL |
| 2139 | 728131 | RTA22200014F.o.08.2.P.Seq | F | M00057024:75 | CH16COP |
| 2140 | 475203 | RTA22200014F.m.07.2.P.Seq | F | M00057011:43 | CH16COP |
| 2141 | 724616 | RTA22200011F.l.16.1.P.Seq | F | M00056622:33 | CH16COP |
| 2142 | 726594 | RTA22200015F.d.13.1.P.Seq | F | M00057066:14 | CH16COP |
| 2143 | 645222 | RTA22200008F.a.16.1.P.Seq | F | M00056300:15 | CH15CON |
| 2144 | 400362 | RTA22200024F.j.22.1.P.Seq | F | M00055222:85 | CH17COHLV |
| 2145 | 646583 | RTA22200009F.m.15.1.P.Seq | F | M00042850:310 | CH16COP |
| 2146 | 475203 | RTA22200014F.m.07.1.P.Seq | F | M00057011:43 | CH16COP |
| 2147 | 550001 | RTA22200002F.e.03.1.P.Seq | F | M00055454:17 | CH15CON |
| 2148 | 640703 | RTA22200002F.i.19.1.P.Seq | F | M00055512:47 | CH15CON |
| 2149 | 646583 | RTA22200007F.k.10.2.P.Seq | F | M00056236:71 | CH15CON |
| 2150 | 449468 | RTA22200014F.l.23.1.P.Seq | F | M00057008:59 | CH16COP |
| 2151 | 449468 | RTA22200016F.h.22.1.P.Seq | F | M00057223:36 | CH16COP |
| 2152 | 551628 | RTA22200001F.l.16.1.P.Seq | F | M00042891:34 | CH15CON |
| 2153 | 449468 | RTA22200014F.l.23.2.P.Seq | F | M00057008:59 | CH16COP |
| 2154 | 417259 | RTA22200021F.i.19.3.P.Seq | F | M00054804:812 | CH17COHLV |
| 2155 | 448029 | RTA22200009F.n.10.1.P.Seq | F | M00042856:67 | CH15COP |
| 2156 | 524363 | RTA22200024F.h.07.1.P.Seq | F | M00055201:13 | CH17COHLV |
| 2157 | 446531 | RTA22200023F.n.24.1.P.Seq | F | M00055116:22 | CH17COHLV |
| 2158 | 561359 | RTA22200008F.a.11.1.P.Seq | F | M00056295:67 | CH15CON |
| 2159 | 711297 | RTA22200007F.f.10.1.P.Seq | F | M00056190:72 | CH15CON |
| 2160 | 650097 | RTA22200015F.g.20.1.P.Seq | F | M00057088:32 | CH16COP |
| 2161 | 495715 | RTA22200021F.m.16.2.P.Seq | F | M00054839:22 | CH17COHLV |
| 2162 | 734685 | RTA22200014F.i.17.1.P.Seq | F | M00056986:71 | CH16COP |
| 2163 | 560515 | RTA22200023F.g.10.1.P.Seq | F | M00055054:32 | CH17COHLV |
| 2164 | 3441 | RTA22200235F.l.18.2.P.Seq | F | M00022537:311 | CH03MAH |
| 2165 | 729273 | RTA22200016F.i.03.1.P.Seq | F | M00057224:310 | CH16COP |
| 2166 | 557039 | RTA22200004F.g.15.1.P.Seq | F | M00055786:45 | CH15CON |
| 2167 | 711297 | RTA22200015F.k.22.1.P.Seq | F | M00057114:210 | CH16COP |
| 2168 | 711297 | RTA22200014F.g.08.2.P.Seq | F | M00056967:42 | CH16COP |
| 2169 | 2860 | RTA22200012F.i.07.1.P.Seq | F | M00056726:75 | CH16COP |
| 2170 | 558534 | RTA22200020F.l.05.1.P.Seq | F | M00054665:511 | CH17COHLV |
| 2171 | 711297 | RTA22200014F.g.08.1.P.Seq | F | M00056967:42 | CH16COP |
| 2172 | 378457 | RTA22200025F.g.08.1.P.Seq | F | M00055338:87 | CH17COHLV |
| 2173 | 646583 | RTA22200007F.n.04.1.P.Seq | F | M00056263:31 | CH15CON |
| 2174 | 646583 | RTA22200006F.p.06.2.P.Seq | F | M00056128:210 | CH15CON |
| 2175 | 1996 | RTA22200012F.m.23.1.P.Seq | F | M00056762:55 | CH16COP |
| 2176 | 7962 | RTA22200235F.d.18.1.P.Seq | F | M00022452:27 | CH03MAH |
| 2177 | 645139 | RTA22200013F.n.18.1.P.Seq | F | M00056902:812 | CH16COP |
| 2178 | 449468 | RTA22200018F.g.12.1.P.Seq | F | M00043313:13 | CH17COHLV |
| 2179 | 9898 | RTA22200238F.f.06.1.P.Seq | F | M00022902:810 | CH03MAH |
| 2180 | 406499 | RTA22200004F.i.15.1.P.Seq | F | M00055797:57 | CH15CON |
| 2181 | 1257 | RTA22200230F.h.06.1.P.Seq | F | M00007156.68 | CH02COH |
| 2182 | 549903 | RTA22200004F.g.12.1.P.Seq | F | M00055785:58 | CH15CON |
| 2183 | 557906 | RTA22200004F.j.22.1.P.Seq | F | M00055803:48 | CH15CON |
| 2184 | 3538 | RTA22200222F.c.09.1.P.Seq | F | M00001491:38 | CH01COH |
| 2185 | 3114 | RTA22200231F.n.12.1.P.Seq | F | M00007994:311 | CH03MAH |
| 2186 | 426895 | RTA22200018F.l.24.1.P.Seq | F | M00043352:25 | CH17COHLV |
| 2187 | 923 | RTA22200015F.c.15.1.P.Seq | F | M00057060:112 | CH16COP |
| 2188 | 645194 | RTA22200007F.h.17.1.P.Seq | F | M00056212:71 | CH15C0N |
| 2189 | 550161 | RTA22200014F.a.06.1.P.Seq | F | M00056921:37 | CH16COP |
| 2190 | 650119 | RTA22200005F.d.21.1.P.Seq | F | M00055874:43 | CH15CON |
| 2191 | 642142 | RTA22200016F.g.21.1.P.Seq | F | M00057217:27 | CH16COP |
| 2192 | 419255 | RTA22200004F.l.20.1.P.Seq | F | M00055814:411 | CH15CON |
| 2193 | 552905 | RTA22200019F.j.20.1.P.Seq | F | M00054518:43 | CH17COHLV |
| 2194 | 511997 | RTA22200005F.b.22.1.P.Seq | F | M00055860:18 | CH15CON |
| 2195 | 551434 | RTA22200003F.m.02.1.P.Seq | F | M00055701:410 | CH15CON |
| 2196 | 727447 | RTA22200012F.m.18.1.P.Seq | F | M00056759:73 | CH16COP |
| 2197 | 378786 | RTA22200025F.o.11.2.P.Seq | F | M00055396:77 | CH17COHLV |
| 2198 | 649152 | RTA22200006F.p.11.2.P.Seq | F | M00056131:312 | CH15CON |
| 2199 | 18853 | RTA22200023F.p.10.1.P.Seq | F | M00055134:53 | CH17COHLV |
| 2200 | 643481 | RTA22200005F.f.05.1.P.Seq | F | M00055884:510 | CH15CON |
| 2201 | 644417 | RTA22200004F.p.08.1.P.Seq | F | M00055838:712 | CH15CON |
| 2202 | 726788 | RTA22200011F.i.17.1.P.Seq | F | M00056597:32 | CH16COP |
| 2203 | 206 | RTA22200248F.m.13.1.P.Seq | F | M00027594:33 | CH04MAL |
| 2204 | 395930 | RTA22200011F.c.20.1.P.Seq | F | M00056548:311 | CH16COP |
| 2205 | 185589 | RTA22200244F.a.15.1.P.Seq | F | M00027168:810 | CH04MAL |
| 2206 | 1441 | RTA22200225F.i.15.1.P.Seq | F | M00005485:25 | CH02COH |
| 2207 | 14522 | RTA22200241F.h.19.1.P.Seq | F | M00026909:73 | CH04MAL |
| 2208 | 203605 | RTA22200013F.n.02.1.P.Seq | F | M00056896:65 | CH16COP |
| 2209 | 551527 | RTA22200019F.g.15.1.P.Seq | F | M00054496:29 | CH17COHLV |
| 2210 | 4509 | RTA22200225F.m.07.1.P.Seq | F | M00005514:16 | CH02COH |
| 2211 | 447737 | RTA22200001F.g.12.1.P.Seq | F | M00042704:69 | CH15CON |
| 2212 | 447388 | RTA22200004F.k.08.1.P.Seq | F | M00055804:61 | CH15CON |
| 2213 | 451932 | RTA22200002F.c.24.1.P.Seq | F | M00055447:84 | CH15CON |
| 2214 | 559043 | RTA22200025F.c.20.1.P.Seq | F | M00055294:44 | CH17COHLV |
| 2215 | 380634 | RTA22200021F.n.06.2.P.Seq | F | M00054842:311 | CH17COH |
| 2216 | 495715 | RTA22200018F.p.18.1.P.Seq | F | M00043379:37 | CH17COHLV |
| 2217 | 451932 | RTA22200002F.d.01.1.P.Seq | F | M00055447:84 | CH15CON |
| 2218 | 447939 | RTA22200002F.d.12.1.P.Seq | F | M00055450:79 | CH15CON |
| 2219 | 1181 | RTA22200250F.c.03.1.P.Seq | F | M00027781:54 | CH04MAL |
| 2220 | 376600 | RTA22200017F.a.O6.1.P.Seq | F | M00057288:58 | CH16COP |
| 2221 | 234761 | RTA22200001F.j.10.1.P.Seq | F | M00042739:41 | CH15CON |
| 2222 | 644417 | RTA22200014F.d.20.1.P.Seq | F | M00056952:16 | CH16COP |
| 2223 | 639048 | RTA22200004F.c.05.1.P.Seq | F | M00055749:811 | CH15CON |
| 2224 | 11452 | RTA22200226F.d.16.1.P.Seq | F | M00005628:310 | CH02COH |
| 2225 | 452076 | RTA22200001F.c.17.1.P.Seq | F | M00042551:49 | CH15CON |
| 2226 | 644523 | RTA22200005F.c.07.1.P.Seq | F | M00055864:82 | CH15CON |
| 2227 | 554678 | RTA22200021F.h.11.3.P.Seq | F | M00054776:61 | CH17COHLV |
| 2228 | 3550 | RTA22200236F.k.16.1.P.Seq | F | M00022656:511 | CH03MAH |
| 2229 | 450311 | RTA22200007F.d.12.1.P.Seq | F | M00056167:28 | CH15CON |
| 2230 | 647280 | RTA22200005F.o.13.1.P.Seq | F | M00055976:61 | CH15CON |
| 2231 | 548858 | RTA22200026F.f.18.1.P.Seq | F | M00055480:810 | CH17COHLV |
| 2232 | 4204 | RTA22200225F.p.22.1.P.Seq | F | M00005568:29 | CH02COH |
| 2233 | 540690 | RTA22200011F.f.19.1.P.Seq | F | M00056574:17 | CH16COP |
| 2234 | 404774 | RTA22200009F.k.06.2.P.Seq | F | M00042833:71 | CH16COP |
| 2235 | 557823 | RTA22200023F.h.01.1.P.Seq | F | M00055057:63 | CH17COHLV |
| 2236 | 1458 | RTA22200242F.g.04.1.P.Seq | F | M00027014:74 | CH04MAL |
| 2237 | 485431 | RTA22200020F.e.01.1.P.Seq | F | M00054595:89 | CH17COHLV |
| 2238 | 2245 | RTA22200229F.l.11.1.P.Seq | F | M00007032:69 | CH02COH |
| 2239 | 3242 | RTA22200226F.p.09.1.P.Seq | F | M00005826:710 | CH02COH |
| 2240 | 648747 | RTA22200007F.m.12.1.P.Seq | F | M00056254:82 | CH15CON |
| 2241 | 3805 | RTA22200222F.c.01.1.P.Seq | F | M00001470:71 | CH01COH |
| 2242 | 475203 | RTA22200012F.c.17.1.P.Seq | F | M00056688:65 | CH16COP |
| 2243 | 12018 | RTA22200240F.h.11.1.P.Seq | F | M00023399:38 | CH04MAL |
| 2244 | 475203 | RTA22200009F.i.18.2.P.Seq | F | M00042818:512 | CH16COP |
| 2245 | 3805 | RTA22200222F.b.24.1.P.Seq | F | M00001470:71 | CH01COH |
| 2246 | 496132 | RTA22200018F.e.23.1.P.Seq | F | M00043304:41 | CH17COHLV |
| 2247 | 650600 | RTA22200005F.m.01.1.P.Seq | F | M00055941:212 | CH15CON |
| 2248 | 650749 | RTA22200004F.g.10.1.P.Seq | F | M00055785:63 | CH15CON |
| 2249 | 223148 | RTA22200023F.n.05.1.P.Seq | F | M00055106:47 | CH17COHLV |
| 2250 | 449 | RTA22200018F.m.12.1.P.Seq | F | M00043355:811 | CH17COHLV |
| 2251 | 735620 | RTA22200013F.g.07.1.P.Seq | F | M00056843:89 | CH16COP |
| 2252 | 650600 | RTA22200005F.l.24.1.P.Seq | F | M00055941:212 | CH15CON |
| 2253 | 218 | RTA22200021F.m.03.2.P.Seq | F | M00054832:59 | CH17COHLV |
| 2254 | 4161 | RTA22200222F.d.07.1.P.Seq | F | M00001561:71 | CH01COH |
| 2255 | 373202 | RTA22200025F.f.01.1.P.Seq | F | M00055322:51 | CH17COHLV |
| 2256 | 724339 | RTA22200010F.l.14.1.P.Seq | F | M00056490:52 | CH16COP |
| 2257 | 133291 | RTA22200007F.p.17.1.P.Seq | F | M00056290:65 | CH15CON |
| 2258 | 736753 | RTA22200016F.f.22.1.P.Seq | F | M00057208:48 | CH16C0P |
| 2259 | 650600 | RTA22200003F.m.18.1.P.Seq | F | M00055706:11 | CH15CON |
| 2260 | 451569 | RTA22200013F.l.03.1.P.Seq | F | M00056879:12 | CH16COP |
| 2261 | 1297 | RTA22200233F.n.06.1.P.Seq | F | M00021655:82 | CH03MAH |
| 2262 | 3680 | RTA22200241F.h.12.1.P.Seq | F | M00026906:310 | CH01MAL |
| 2263 | 63602 | RTA22200005F.g.11.1.P.Seq | F | M00055888:25 | CH15CON |
| 2264 | 2757 | RTA22200238F.l.07.1.P.Seq | F | M00022973:77 | CH03MAH |
| 2265 | 373128 | RTA22200020F.a.11.1.P.Seq | F | M00054569:87 | CH17COHLV |
| 2266 | 641479 | RTA22200014F.l.16.2.P.Seq | F | M00057007:212 | CH16COP |
| 2267 | 450380 | RTA22200015F.b.12.1.P.Seq | F | M00057049:85 | CH16COP |
| 2268 | 133512 | RTA22200024F.i.12.1.P.Seq | F | M00055209:18 | CH17COHLV |
| 2269 | 447211 | RTA22200006F.b.19.2.P.Seq | F | M00056007:66 | CH15CON |
| 2270 | 645222 | RTA22200013F.a.24.1.P.Seq | F | M00056802:81 | CH16COP |
| 2271 | 645222 | RTA22200013F.b.01.1.P.Seq | F | M00056802:81 | CH16COP |
| 2272 | 17372 | RTA22200002F.i.22.1.P.Seq | F | M0055513:46 | CH15CON |
| 2273 | 451619 | RTA22200025F.m.23.2.P.Seq | F | M00055388:88 | CH17COHLV |
| 2274 | 2510 | RTA22200238F.m.21.1.P.Seq | F | M00022995:52 | CH03MAH |
| 2275 | 643974 | RTA22200007F.n.15.1.P.Seq | F | M00056268:24 | CH15CON |
| 2276 | 500630 | RTA22200013F.j.19.1.P.Seq | F | M00056872:16 | CH16COP |
| 2277 | 3101 | RTA22200225F.n.05.1.P.Seq | F | M00005530:17 | CH02COH |
| 2278 | 446938 | RTA22200004F.c.11.1.P.Seq | F | M00055751:41 | CH15CON |
| 2279 | 554469 | RTA22200013F.f.23.1.P.Seq | F | M00056841:79 | CH16COP |
| 2280 | 554469 | RTA222000O3F.n.24.1.P.Seq | F | M00055721:66 | CH15CON |
| 2281 | 2894 | RTA22200016F.b.05.1.P.Seq | F | M00057163:21 | CH16COP |
| 2282 | 650600 | RTA22200008F.e.04.1.P.Seq | F | M00056338:210 | CH15CON |
| 2283 | 3101 | RTA22200226F.j.12.1.P.Seq | F | M00005710:41 | CH02COH |
| 2284 | 554469 | RTA22200003F.o.01.1.P.Seq | F | M00055721:66 | CH15CON |
| 2285 | 9910 | RTA22200236F.n.06.1.P.Seq | F | M00022667:35 | CH03MAH |
| 2286 | 400608 | RTA22200008F.g.03.1.P.Seq | F | M00056460:711 | CH15CON |
| 2287 | 555051 | RTA22200021F.o.07.2.P.Seq | F | M00054854:58 | CH17COHLV |
| 2288 | 185400 | RTA22200002F.j.21.1.P.Seq | F | M00055522:57 | CH15CON |
| 2289 | 3059 | RTA22200233F.g.05.1.P.Seq | F | M00008075:112 | CH03MAH |
| 2290 | 647185 | RTA22200004F.b.20.1.P.Seq | F | M00055747:49 | CH15CON |
| 2291 | 1669 | RTA22200237F.p.19.1.P.Seq | F | M00022853:311 | CH03MAH |
| 2292 | 7158 | RTA22200232F.l.21.1.P.Seq | F | M00022129:512 | CH03MAH |
| 2293 | 496132 | RTA22200005F.d.06.1.P.Seq | F | M00055871:76 | CH15CON |
| 2294 | 378623 | RTA33300035F.k.20.1.P.Seq | F | M00055227:52 | CH17COHLV |
| 2295 | 1257 | RTA22200227F.i.05.1.P.Seq | F | M00006679:34 | CH02COH |
| 2296 | 648499 | RTA22200012F.d.21.1.P.Seq | F | M00056698:512 | CH16COP |
| 2297 | 185627 | RTA22200242F.k.21.1.P.Seq | F | M00027042:711 | CH04MAL |
| 2298 | 640005 | RTA22200008F.c.15.1.P.Seq | F | M00056312:33 | CH15CON |
| 2299 | 553462 | RTA22200021F.e.14.3.P.Seq | F | M00054750:412 | CH17COHLV |
| 2300 | 649852 | RTA22200002F.g.01.1.P.Seq | F | M00055470:71 | CH15CON |
| 2301 | 422375 | RTA22200018F.e.04.1.P.Seq | F | M00043296:210 | CH17COHLV |
| 2302 | 10910 | RTA22200227F.n.22.1.P.Seq | F | M00006745:42 | CH02COH |
| 2303 | 2737 | RTA22200023F.c.21.1.P.Seq | F | M00055034:71 | CH17COHLV |
| 2304 | 3438 | RTA22200225F.o.18.1.P.Seq | F | M00005548:12 | CH02COH |
| 2305 | 3438 | RTA22200228F.m.18.2.P.Seq | F | M00006885:711 | CH02COH |
| 2306 | 3763 | RTA22200225F.o.12.1.P.Seq | F | M00005546:72 | CH02COH |
| 2307 | 648966 | RTA22200007F.a.18.1.P.Seq | F | M00056141:29 | CH15C0N |
| 2308 | 724339 | RTA22200013F.m.10.1.P.Seq | F | M00056892:11 | CH16COP |
| 2309 | 451569 | RTA22200018F.l.17.1.P.Seq | F | M00043350:86 | CH17COHLV |
| 2310 | 554109 | RTA22200007F.d.17.1.P.Seq | F | M0056171:812 | CH15CON |
| 2311 | 380339 | RTA22200006F.h.10.2.P.Seq | F | M00056055:17 | CH15CON |
| 2312 | 729903 | RTA22200014F.g.06.1.P.Seq | F | M00056966:111 | CH16COP |
| 2313 | 45 | RTA22200025F.l.10.1.P.Seq | F | M00055379:38 | CH17COHLV |
| 2314 | 454653 | RTA22200019F.d.21.1.P.Seq | F | M00043503:55 | CH17COHLV |
| 2315 | 11536 | RTA22200237F.n.02.1.P.Seq | F | M00022828:36 | CH03MAH |
| 2316 | 373134 | RTA22200001F.h.02.1.P.Seq | F | M00042711:34 | CH15CON |
| 2317 | 185691 | RTA22200241F.g.04.1.P.Seq | F | M00026900:32 | CH04MAL |
| 2318 | 234761 | RTA22200011F.o.06.1.P.Seq | F | M00056646:75 | CH16COP |
| 2319 | 724339 | RTA22200012F.f.19.1.P.Seq | F | M00056710:31 | CH16COP |
| 2320 | 732740 | RTA22200016F.j.04.1.P.Seq | F | M00057231:19 | CH16COP |
| 2321 | 35895 | RTA22200017F.d.10.1.P.Seq | F | M00057325:310 | CH16COP |
| 2322 | 133512 | RTA22200018F.d.16.1.P.Seq | F | M00042517:67 | CH17COHLV |
| 2323 | 2974 | RTA22200227F.b.03.1.P.Seq | F | M00006590:310 | CH02COH |
| 2324 | 500 | RTA22200231F.l.13.1.P.Seq | F | M00007983:43 | CH03MAH |
| 2325 | 376919 | RTA22200020F.m.09.1.P.Seq | F | M00054678:57 | CH17COHLV |
| 2326 | 8403 | RTA22200233F.g.08.1.P.Seq | F | M00008076:62 | CH03MAH |
| 2327 | 3643 | RTA22200227F.e.11.1.P.Seq | F | M00006638:72 | CH02COH |
| 2328 | 447211 | RTA22200001F.j.13.1.P.Seq | F | M00042741:410 | CH15CON |
| 2329 | 447211 | RTA22200001F.b.15.1.P.Seq | F | M00042538:18 | CH15CON |
| 2330 | 14929 | RTA22200025F.i.20.1.P.Seq | F | M00055363:712 | CH17COHLV |
| 2331 | 648934 | RTA22200006F.k.14.2.P.Seq | F | M0005081:29 | CH15CON |
| 2332 | 731785 | RTA22200014F.j.11.2.P.Seq | F | M00056993:43 | CH16COP |
| 2333 | 639908 | RTA22200005F.g.08.1.P.Seq | F | M00055887:54 | CH15CON |
| 2334 | 344577 | RTA22200014F.m.20.1.P.Seq | F | M00057015:312 | CH16COP |
| 2335 | 2906 | RTA22200015F.m.13.1.P.Seq | F | M00057127:611 | CH16COP |
| 2336 | 446938 | RTA22200002F.c.18.1.P.Seq | F | M00055445:76 | CH15CON |
| 2337 | 2493 | RTA22200234F.h.08.1.P.Seq | F | M00022251:19 | CH03MAH |
| 2338 | 38 | RTA22200006F.o.08.2.P.Seq | F | M00056112:82 | CH15CON |
| 2339 | 13818 | RTA22200238F.e.13.1.P.Seq | F | M00022898:64 | CH03MAH |
| 2340 | 8371 | RTA22200229F.o.12.1.P.Seq | F | M00007065:611 | CH02COH |
| 2341 | 402494 | RTA22200012F.o.16.1.P.Seq | F | M00056776:16 | CH16COP |
| 2342 | 731785 | RTA22200014F.j.11.1.P.Seq | F | M00056993:43 | CH16COP |
| 2343 | 4621 | RTA22200235F.f.15.1.P.Seq | F | M00022468:510 | CH03MAH |
| 2344 | 9750 | RTA22200229F.g.23.1.P.Seq | F | M00006979:210 | CH02COH |
| 2345 | 133512 | RTA22200024F.p.23.1.P.Seq | F | M00055263:79 | CH17COHLV |
| 2346 | 162626 | RTA22200250F.f.03.1.P.Seq | F | M00027823:77 | CH04MAL |
| 2347 | 730059 | RTA22200011F.a.20.1.P.Seq | F | M00056530:41 | CH16COP |
| 2348 | 2069 | RTA22200224F.k.12.1.P.Seq | F | M00005373:86 | CH02COH |
| 2349 | 5868 | RTA22200242F.i.17.1.P.Seq | F | M00027030:38 | CH04MAL |
| 2350 | 2683 | RTA22200226F.o.08.1.P.Seq | F | M00005813:510 | CH02COH |
| 2351 | 380409 | RTA22200012F.h.07.1.P.Seq | F | M00056719:612 | CH16COP |
| 2352 | 639991 | RTA22200002F.k.20.1.P.Seq | F | M00055527:54 | CH15CON |
| 2353 | 535 | RTA22200233F.a.19.1.P.Seq | F | M00008015:210 | CH03MAH |
| 2354 | 14929 | RTA22200025F.f.21.1.P.Seq | F | M00055335:83 | CH17COHLV |
| 2355 | 134702 | RTA22200249F.n.23.1.P.Seq | F | M00027733:45 | CH04MAL |
| 2356 | 642477 | RTA22200004F.j.16.1.P.Seq | F | M00055802:612 | CH15CON |
| 2357 | 14929 | RTA22200014F.i.15.2.P.Seq | F | M00056986:611 | CH16COP |
| 2358 | 134702 | RTA22200241F.n.23.1.P.Seq | F | M00026951:76 | CH04MAL |
| 2359 | 185649 | RTA22200250F.g.08.1.P.Seq | F | M00027833:41 | CH04MAL |
| 2360 | 10702 | RTA22200241F.b.05.1.P.Seq | F | M00026860:51 | CH04MAL |
| 2361 | 643955 | RTA22200001F.k.21.1.P.Seq | F | M00042886:33 | CH15CON |
| 2362 | 643955 | RTA22200004F.p.19.1.P.Seq | F | M00055841:29 | CH15CON |
| 2363 | 4455 | RTA22200233F.o.08.1.P.Seq | F | M00021670:75 | CH03MAH |
| 2364 | 185567 | RTA22200242F.c.16.1.P.Seq | F | M00026985:25 | CH04MAL |
| 2365 | 9115 | RTA22200226F.e.04.1.P.Seq | F | M00005632:82 | CH02COH |
| 2366 | 14929 | RTA22200015F.i.12.1.P.Seq | F | M00057100:59 | CH16COP |
| 2367 | 14929 | RTA22200014F.i.15.1.P.Seq | F | M00056986:611 | CH16COP |
| 2368 | 4181 | RTA22200244F.k.18.1.P.Seq | F | M00027203:88 | CH04MAL |
| 2369 | 5206 | RTA22200238F.n.19.1.P.Seq | F | M00023002:710 | CH03MAH |
| 2370 | 825 | RTA22200237F.c.05.1.P.Seq | F | M00022702:24 | CH03MAH |
| 2371 | 825 | RTA22200238F.o.24.1.P.Seq | F | M00023020:79 | CH03MAH |
| 2372 | 825 | RTA22200231F.d.09.1.P.Seq | F | M00007943:32 | CH03MAH |
| 2373 | 2748 | RTA22200225F.d.18.1.P.Seq | F | M00005449:510 | CH02COH |
| 2374 | 2748 | RTA22200238F.i.02.1.P.Seq | F | M00022934:23 | CH03MAH |
| 2375 | 2748 | RTA22200225F.d.08.1.P.Seq | F | M00005445:12 | CH02COH |
| 2376 | 133512 | RTA22200014F.a.15.2.P.Seq | F | M00056923:59 | CH16COP |
| 2377 | 2748 | RTA22200237F.e.09.1.P.Seq | F | M00022719:612 | CH03MAH |
| 2378 | 642477 | RTA22200008F.e.02.1.P.Seq | F | M00056338:26 | CH15CON |
| 2379 | 642477 | RTA22200007F.n.16.1.P.Seq | F | M00056268:41 | CH15CON |
| 2380 | 2493 | RTA22200236F.f.17.1.P.Seq | F | M00022624:32 | CH03MAH |
| 2381 | 5796 | RTA22200235F.j.03.2.P.Seg | F | M00022508:32 | CH03MAH |
| 2382 | 3782 | RTA22200236F.k.02.1.P.Seq | F | M00022655:410 | CH03MAH |
| 2383 | 884 | RTA22200016F.b.01.1.P.Seq | F | M00057162:82 | CH16COP |
| 2384 | 5860 | RTA22200238F.p.04.1.P.Seq | F | M00023023:63 | CH03MAH |
| 2385 | 5275 | RTA22200226F.h.09.1.P.Seq | F | M00005675:72 | CH02COH |
| 2386 | 3932 | RTA22200233F.l.12.1.P.Seq | F | M00021626:34 | CH03MAH |
| 2387 | 884 | RTA22200017F.a.17.1.P.Seq | F | M00057297:63 | CH16COP |
| 2388 | 4455 | RTA22200235F.j.23.2.P.Seq | F | M00022517:53 | CH03MAH |
| 2389 | 5860 | RTA22200236F.c.15.1.P.Seq | F | M00022598:45 | CH03MAH |
| 2390 | 5860 | RTA22200232F.m.23.1.P.Seq | F | M00022143:310 | CH03MAH |
| 2391 | 372791 | RTA22200003F.o.02.1.P.Seq | F | M00055721:55 | CH15CON |
| 2392 | 5206 | RTA22200238F.o.14.1.P.Seq | F | M00023015:410 | CH03MAH |
| 2393 | 372791 | RTA22200005F.o.07.1.P.Seq | F | M00055973:44 | CH15CON |
| 2394 | 2846 | RTA22200228F.l.12.2.P.Seq | F | M00006873:512 | CH02COH |
| 2395 | 5275 | RTA22200225F.g.17.1.P.Seq | F | M00005469:17 | CH02COH |
| 2396 | 2846 | RTA22200228F.i.21.2.P.Seq | F | M00006852:37 | CH02COH |

TABLE 2B
Nearest Neighbor (BlastX vs. Non-Redundant Proteins)

| SEQ ID | ACCESSION | DESCRIPTION1 | P VALUE |
|---|---|---|---|
| 63 | 3236249 | (AC004684) hypothetical protein [Arabidopsis thaliana] | 9.9 |
| 64 | 123111 | H-2 CLASS II HISTOCOMPATIBILITY ANTIGEN, GAMMA | 9.7 |
| | | CHAIN (HLA-DR ANTIGENS ASSOCIATED INVARIANT | |
| | | CHAIN) >gi\|92086\|pir\|\|S04362 class II histocompatibility antigen- | |
| | | associated gamma chain, long splice form - rat | |
| 65 | 94377 | hypothetical protein 221 - turnip yellow mosaic virus | 9.6 |
| 66 | 2736449 | (AF039047) contains similarity to the BPTI/kunitz family of | 9.5 |
| | | inhibitors [Caenorhabditis elegans] | |
| 67 | 543894 | BETA-LACTAMASE PRECURSOR | 8.2 |
| 68 | 3881525 | (Z70038) cDNA EST EMBL:D32579 comes from this gene; cDNA | 7.6 |
| | | EST EMBL:D35254 comes from this gene; cDNA EST yk224b3.5 | |
| | | comes from this gene; cDNA EST yk357f10.5 comes from this gene | |
| 69 | 3128358 | (AF010496) ribose transport system permease protein RbsC | 7.5 |
| 70 | 3875771 | (Z68297) Weak similarity to Mouse DNA-binding protein BMI-1 | 7.3 |
| | | (SW:BMI1_MOUSE); cDNA EST EMBL:C07407 comes from this | |
| | | gene; cDNA EST EMBL:C07408 comes from this gene | |
| 71 | 21293 | (Y00759) 20 kDa protein (AA 1-212) [Spinacia oleracea] | 7.3 |
| 72 | 2131007 | (Z95890) pknE [Mycobacterium tuberculosis] | 7.2 |
| 73 | 245923 | (S83583) multiple-epitope polypeptide 1, MEP-1 construct] | 7 |
| 74 | 115347 | PROCOLLAGEN ALPHA 2(IV) CHAIN PRECURSOR | 6.9 |
| | | >gi\|84486\|pir\|\|S16366 collagen alpha 2(IV) chain precursor - pig | |
| | | roundworm >gi\|159649 (M67507) putative [Ascaris suum] | |
| 75 | 2072674 | (Z95120) rhlE [Mycobacterium tuberculosis] | 5.8 |
| 76 | 807646 | (M17294) unknown protein [Human herpesvirus 4] | 5.8 |
| 77 | 131706 | URID1NE 5′-MONOPHOSPHATE SYNTHASE OROTIDINE 5′- | 5.6 |
| | | PHOSPHATE DECARBOXYLASE | |
| 78 | 631593 | glucose transport protein homolog - sheep | 5 |
| 79 | 854064 | (X83413) U87 [Human herpesvirus 6] | 4.8 |
| 80 | 484695 | vascular cell adhesion molecule 1 - human | 4.2 |
| 81 | 1236146 | (U49864) fus 1 protein [Chlamydomonas reinhardtii] | 3.8 |
| 82 | 1083846 | acetyl-CoA carboxylase (EC 6.4.1.2) - Cyclotella cryptica >gi\|409450 | 3.5 |
| | | (L20784) acetyl-CoA carboxylase [Cyclotella cryptica] | |
| 83 | 2245054 | (Z97342) protein kinase homolog | 3.4 |
| 84 | 1717863 | UBIQUITIN-CONJUGATING ENZYME E2-21.2 KD (UBIQUITIN- | 3.4 |
| | | PROTEIN LIGASE) (UBIQUITIN CARRIER PROTEIN) | |
| | | >gi\|1077331\|pir\|\|S51438 probable membrane protein YLR306w - | |
| 85 | 1363331 | transcription factor IIIC alpha chain - rat alpha-subunit [Rattus | 3.4 |
| 86 | 2736327 | (AF038615) No definition line found [Caenorhabditis elegans] | 3.1 |
| 87 | 4587895 | (AF072509) glutamate receptor interacting protein 2 [Rattus | 3.1 |
| 88 | 1480746 | (U62529) matrix metalloproteinase 3 [Equus caballus] | 3 |
| 89 | 225858 | thyroid/steroid receptor related gene [Homo sapiens] | 3 |
| 90 | 3861293 | (AJ235273) 3-OXOACYL-[ACYL-CARRIER-PROTEIN] | 2.4 |
| 91 | 103812 | hypothetical protein 1 - zebra fish | 2 |
| 92 | 3256583 | (AP000001) 361aa long hypothetical protein [Pyrococcus horikoshii] | 1.7 |
| 93 | 1684985 | (U20633) NADH dehydrogenase subunit [Neuwiedia veratrifolia] | 1.5 |
| 94 | 452517 | (D26361) KIAA0042 [Homo sapiens] | 1.4 |
| 95 | 4218558 | (AJ011500) gra-orf26 [Streptomyces violaceoruber] | 1.1 |
| 96 | 4539280 | (AL049498) putative transcription factor | 0.28 |
| 97 | 4539280 | (AL049498) putative transcription factor | 0.26 |
| 98 | 3881842 | (Z78201) Similarity to E.coli 2-oxoglutarate dehydrogenase | 4e−019 |
| | | (SW:ODO1_ECOLI); cDNA EST EMBL:D32590 comes from this | |
| | | gene; cDNA EST EMBL:D32841 comes from this gene; cDNA EST | |
| | | EMBL:D34051 comes from this gene; cDNA EST EMBL:D35268 | |
| 99 | 4102877 | (AF017152) Shc binding protein [Mus musculus] | 6e−050 |
| 100 | 3327062 | (AB014524) KIAA0624 protein [Homo sapiens] | 5e−050 |
| 102 | 3126979 | (AF062483) SDP3 [Homo sapiens] | 6.1 |
| 103 | 3930776 | (AF099149) TRIAD1 type I [Homo sapiens] | 9e−068 |
| 106 | 2708741 | (AC003952) hypothetical protein [Arabidopsis thaliana] | 3.6 |
| 107 | 733554 | (U23450) similar to RNA-binding protein [Caenorhabditis elegans] | 1e−014 |
| 108 | 3851703 | (AF100421) p80 [Rattus norvegicus] | 7e−055 |
| 112 | 2120729 | GumG protein - Xanthomonas campestris | 6 |
| 120 | 3334526 | (AL021306) predicted using FGENEH [Homo sapiens] | 7.7 |
| 121 | 2407956 | (X87612) mono ATP-ribosaltransferase [Mus musculus] | 5.9 |
| 122 | 3877701 | (Z69662) predicted using Genefinder; similar to collagen; cDNA | 5.9 |
| | | EST EMBL:D75049 comes from this gene; cDNA EST | |
| | | EMBL:D72067 comes from this gene; cDNA EST EMBL:D72223 | |
| | | comes from this gene; cDNA EST EMBL:D72768 comes from this | |
| 123 | 2117780 | serine/threonine protein kinase - quail | 3.5 |
| 124 | 1722738 | MINOR CAPSID PROTEIN L2 >gi\|1020224 type 36] | 3.4 |
| 125 | 2494448 | HYPOTHETICAL PROTEIN MJ0208 Methanococcus jannaschii | 3.4 |
| | | >gi\|1498983 (U67476) 4Fe-4S iron-sulfur protein [Methanococcus | |
| 126 | 2494294 | NEUROGENIC LOCUS NOTCH 3 PROTEIN | 3.4 |
| 127 | 4493971 | (AL034559) predicted using hexExon; MAL3P7.11 (PFC0910w), | 0.61 |
| | | Hypothetical protein, len: 430 aa | |
| 138 | 3025005 | HYPOTHETICAL 28.8 KD PROTEIN IN MOAE-RHLE | 9.8 |
| | | INTERGENIC REGION >gi\|1787008 (AE000181) orf, hypothetical | |
| 139 | 3881856 | (Z79759) Similarity to Yeast endosomal P24A protein | 9.6 |
| | | (SW:EM70_YEAST); cDNA EST CEMSB40F comes from this | |
| | | gene; cDNA EST EMBL:C13538 comes from this gene; cDNA EST | |
| 140 | 1945493 | (U56965) Similar to NAD(P) transhydrogenase, mitochondrial; coded | 5.6 |
| | | for by C. elegans cDNA yk27c1.5; coded for by C. elegans cDNA | |
| | | yk35b9.5; coded for by C. elegans cDNA yk35b9.3; coded for by C. | |
| | | elegans cDNA yk161c9.3; coded for by C. elegans ... | |
| 141 | 3746071 | (AC005311) putative GTP-binding protein [Arabidopsis thaliana] | 5.6 |
| 142 | 2088843 | (AF003386) F59E12.9 gene product [Caenorhabditis elegans] | 3.2 |
| 143 | 1706551 | GLUCAN ENDO-1,3-BETA-GLUCOSIDASE PRECURSOR ((1- | 1.9 |
| | | >3)-BETA-GLUCAN ENDOHYDROLASE) aestivum] | |
| 144 | 629778 | chitinase (EC 3.2.1.14) - barley vulgare] | 1.9 |
| 145 | 2791276 | (Z95327) Cleavage Stimulation Factor sapiens] | 0.85 |
| 146 | 736767 | helper component protease [Turnip mosaic virus] | 0.5 |
| 147 | 1731209 | HYPOTHETICAL 35.4 KD PROTEIN CY20G9.19C | 0.22 |
| | | >gi\|1449291\|emb\|CAB00954\|(Z77162) hypothetical protein | |
| 148 | 3877063 | (Z37092) F44F4.10 [Caenorhabditis elegans] | 0.075 |
| 149 | 3878739 | (Z73428) similar to Zinc finger, C3HC4 type (RING finger); cDNA | 2e−029 |
| | | EST EMBL:D67323 comes from this gene [Caenorhabditis elegans] | |
| | | >gi\|3881096\|emb\|CAB16481\|finger); cDNA EST EMBL:D67323 | |
| | | comes from this gene | |
| 159 | 4454483 | (AC006234) putative kinase, 5′ partial | 9.1 |
| 160 | 3044086 | (AF055904) unknown [Myxococcus xanthus] | 5.4 |
| 161 | 1945493 | (U56965) Similar to NAD(P) transhydrogenase, mitochondrial; coded | 5.4 |
| | | for by C. elegans cDNA yk27c1.5; coded for by C. elegans cDNA | |
| | | yk35b9.5; coded for by C. elegans cDNA yk35b9.3; coded for by C. | |
| | | elegans cDNA yk161c9.3; coded for by C. elegans ... | |
| 162 | 730883 | SYNAPTIC VESICLE PROTEIN 2 (SV2) norvegicus] | 5.4 |
| 163 | 2351212 | (D88386) gag-pol polyprotein (precursor protein) [Friend murine | 4.2 |
| | | leukemia virus] | |
| 164 | 1722738 | MINOR CAPSID PROTEIN L2 >gi\|1020224 type 3] | 3.2 |
| 165 | 4115922 | (AF118222) contains similarity to ubiquitin carboxyl-terminal | 2.4 |
| | | hydrolase family 2 (Pfam:PF00443, score = 48.3, E = 3.5e−13, N = 2) | |
| | | and (Pfam:PF00442, Score = 40.0 E = 5.2e−08, N = 1) [Arabidopsis | |
| 166 | 1351639 | VERY HYPOTHETICAL 52.7 KD PROTEIN C8A4.05C IN | 2.4 |
| | | CHROMOSOME I >gi\|2130446\|pir\|\|S62521 hypothetical protein | |
| 167 | 4506857 | small inducible cytokine subfamily D (Cys-X3-Cys), member 1 | 2.4 |
| | | (fractalkine, neurotactin) >gi\|1888523 (U84487) CX3C chemokine | |
| | | precursor [Homo sapiens] >gi\|1899259 (U91835) CX3C chemokine | |
| 168 | 4505637 | protocadherin 8; PCDH8 sapiens] | 1.9 |
| 169 | 3249055 | (AF071210) casein kinase II alpha subunit [Spodoptera frugiperda] | 1.4 |
| 170 | 854065 | (X83413) U88 [Human herpesvirus 6] | 1e−005 |
| 171 | 854065 | (X83413) U88 [Human herpesvirus 6] | 1e−005 |
| 191 | 3150072 | (AF046996) preS1 surface protein [woolly monkey hepatitis B Virus] | 6.9 |
| 192 | 2622845 | (AE000928) corrinoid/iron-sulfur protein, large subunit | 6.9 |
| 193 | 1083554 | tyrosine phosphoprotein SLP-76 - mouse | 6.9 |
| 194 | 1708868 | LOW-DENSITY LIPOPROTEIN RECEPTOR-RELATED | 5.2 |
| | | PROTEIN PRECURSOR (LRP) Caenorhabditis elegans >gi\|156360 | |
| | | (M96150) LDL receptor-related protein [Caenorhabditis elegans] | |
| | | Genefinder; Identity to C.elegans Low density lipid (LDL) receptor- | |
| 195 | 3169030 | (AL023702) putative insertion element IS1647 transposase | 4 |
| | | [Streptomyces coelicolor] | |
| 196 | 728835 | !!!! ALU SUBFAMILY SC WARNING ENTRY | 4 |
| 197 | 484695 | vascular cell adhesion molecule 1 - human | 3.9 |
| 198 | 2204102 | (Y13898) glutathione-S-transferase | 3.9 |
| 199 | 1118071 | (U41554) coded for by C. elegan cDNA yk38a7.3; coded for by C. | 2.3 |
| | | elegans cDNA yk8c6.3; coded for by C. elegans cDNA yk25d12.5; | |
| | | coded for by C. elegans cDNA yk25d12.3; coded for by C. elegans | |
| | | cDNA yk8c6.5; coded for by C. elegans cDNA yk7f8.5;... | |
| 200 | 799146 | (U24495) 2a protein [Broad bean mottle virus] | 1.4 |
| 201 | 73416 | E2 protein - human papillomavirus type 18 papillomavirus type 18] | 1.4 |
| 202 | 294529 | (L14933) convertase PC5 [Rattus norvegicus] | 0.45 |
| 203 | 124141 | TRANS-ACTING TRANSCRIPTIONAL PROTEIN ICP4 | 0.031 |
| | | (TRANSCRIPTIONAL ACTIVATOR IE175) (ALPHA-4 | |
| | | PROTEIN) human herpesvirus 1 >gi\|59558\|emb\|CAA32286\| | |
| | | (X14112) RS1 RS1 [Human herpesvirus 1] | |
| | | >gi\|59849\|emb\|CAA29763\|1298) [Human herpesvirus 1] | |
| 204 | 4503511 | UNKNOWN >gi\|3264861 (U97670) eukaryotic translation initiation | 3e−010 |
| | | factor eIF3, p35 subunit [Homo sapiens] | |
| 205 | 4503511 | UNKNOWN >gi\|3264861 (U97670) eukaryotic translation initiation | 3e−010 |
| | | factor eIF3, p35 subunit [Homo sapiens] | |
| 206 | 3298605 | (AF057365) UDP N-acetylglucosamine transporter [Canis familiaris] | 8e−033 |
| 221 | 3116148 | (AL023290) putative ATP-dependent RNA helicase | 8.7 |
| | | [Schizosaccharomyces pombe] | |
| 222 | 4544400 | (AC007047) hypothetical protein [Arabidopsis thaliana] | 8.7 |
| 223 | 1086982 | E1 replication protein [bovine papillomavirus type 1 BPV-1, Peptide, | 8.6 |
| 224 | 1762434 | (U59924) nitric oxide synthase [Sus scrofa] | 8.6 |
| 225 | 731689 | HYPOTHETICAL 433.2 KD PROTEIN IN HXT5-NRK1 | 8.5 |
| | | INTERGENIC REGION >gi\|626646\|pir\|\|S46715 hypothetical protein | |
| | | YHR099w - yeast (Saccharomyces cerevisiae) >gi\|487929 (U00060) | |
| 226 | 2351132 | (D85200) S glycoprotein [Brassica oleracea] | 8.5 |
| 227 | 1762434 | (U59924) nitric oxide synthase [Sus scrofa] | 8.5 |
| 228 | 4493995 | (AL034559) predicted using hexExon; MAL3P7.47 (PFC1080c), | 6.7 |
| | | Hypothetical protein, len: 232 aa | |
| 229 | 462685 | NUCLEOCAPSID PROTEIN coronavirus (strain K378) | 5.2 |
| | | >gi\|58850\|emb\|CAA47246\| | |
| 230 | 4519268 | (AB024314) CREA [Aspergillus aculeatus] | 5 |
| 231 | 1364138 | probable polymerase - soybean dwarf virus polymerase [Soybean | 5 |
| 232 | 1709938 | ADENYLOSUCCINATE SYNTHETASE adenylosuccinate synthase | 3.8 |
| | | (EC 6.3.4.4) - Thiobacillus ferrooxidans >gi\|48168\|emb\|CAA40593\| | |
| | | (X57324) purA adenylosuccinate synthetase [Thiobacillus | |
| 233 | 1947132 | (AF000262) the second exon has similarity to collagen alpha in a | 2.9 |
| | | glycine- and proline-rich region | |
| 234 | 2245054 | (Z97342) protein kinase homolog | 2.9 |
| 235 | 2736517 | (AF039052) contains similarity to helicases | 1.7 |
| 236 | 1620170 | (U42580) a499L [Paramecium bursaria Chlorella virus 1] | 1.7 |
| 237 | 4336718 | (AF104031) caudal-related homeobox protein | 1.3 |
| 238 | 114969 | BETA-GLUCOSIDASE A (GENTIOBIASE) 3.2.1.21) - | 0.76 |
| | | Caldocellum saccharolyticum 1-455) [Caldicellulosiruptor | |
| 239 | 1170313 | HOMEOBOX PROTEIN CDX-1 (CAUDAL-TYPE HOMEOBOX | 0.44 |
| | | PROTEIN 1) >gi\|1083361\|pir\|\|A49303 homeotic protein Cdx-1 - | |
| 258 | 1172222 | (U44834) polyphosphate glucokinase [Mycobacterium tuberculosis] | 8.4 |
| | | >gi\|1588398\|prf\|\|2208389A phosphate glucokinase [Mycobacterium | |
| 259 | 2959862 | (AJ002303) synaptogyrin 1c [Homo sapiens] | 8.1 |
| 260 | 2983552 | (AE000721) cation efflux system (czcD-like) [Aquifex aeolicus] | 8 |
| 261 | 104800 | nicotinic acetylcholine receptor alpha-5 chain precursor - chicken | 4.8 |
| 262 | 3875957 | (Z78013) predicted using Genefinder; cDNA EST EMBL:D72806 | 4.8 |
| | | comes from this gene; cDNA EST EMBL:D75743 comes from this | |
| | | gene; cDNA EST yk417b6.3 comes from this gene; cDNA EST | |
| | | yk417b6.5 comes from this gene; cDNA EST yk276c6.3 com ... | |
| 263 | 746475 | (U23511) No definition line found [Caenorhabditis elegans] | 4.7 |
| 264 | 1899232 | (U90333) aquarius gene product [Mus musculus] | 2.8 |
| 265 | 3123186 | AFLATOXIN BIOSYNTHESIS REGULATORY PROTEIN | 2.8 |
| 266 | 2500589 | NUCLEOLYSIN TIAR (TIA-1 RELATED PROTEIN) >gi\|1592563 | 2.8 |
| | | (U55861) RNA binding protein TIAR | |
| 267 | 90253 | leukocyte common antigen precursor - mouse musculus] | 1.7 |
| 268 | 2827553 | (AL021635) predicted protein [Arabidopsis thaliana] | 1.6 |
| 269 | 3695397 | (AF096372) No definition line found [Arabidopsis thaliana] | 0.42 |
| 270 | 2564330 | (AB006629) KIAA0291 [Homo sapiens] | 8e−006 |
| 271 | 3413860 | (AB007918) KIAA0449 protein [Homo sapiens] | 3e−009 |
| 272 | 4454698 | (AF070661) HSPC005 [Homo sapiens] | 1e−010 |
| 289 | 266328 | PROBABLE PROCESSING AND TRANSPORT PROTEIN | 6 |
| | | (INFECTED CELL PROTEIN 18.5) murine cytomegalovirus (strain | |
| 290 | 2204102 | (Y13898) glutathione-S-transferase | 3.5 |
| 291 | 2887280 | (AJ002140) DNA | 3.5 |
| 292 | 4567223 | (AC007119) hypothetical protein | 3.1 |
| 303 | 2077849 | (AB003348) E1 protein [Rubella virus] | 9.9 |
| 304 | 845311 | (L40021) polyprotein [Feline calicivirus] | 7.3 |
| 305 | 266328 | PROBABLE PROCESSING AND TRANSPORT PROTEIN | 5.6 |
| | | (INFECTED CELL PROTEIN 18.5) murine cytomegalovirus (strain | |
| 306 | 103469 | homeotic protein bicoid - fruit fly pseudoobscura] | 5.6 |
| 307 | 2204102 | (Y13898) glutathione-S-transferase | 3.3 |
| 308 | 125394 | HOMOSERINE KINASE (HK) Pseudomonas aeruginosa | 3.3 |
| | | >gi\|45425\|emb\|CAA46169\|(X65034) homoserine kinase | |
| | | [Pseudomonas aeruginosa] | |
| 309 | 2353163 | (AF015560) RO11 [Neurospora crassa] | 0.66 |
| 310 | 484695 | vascular cell adhesion molecule 1 - human | 0.64 |
| 311 | 3874146 | (Z72502) Similarity with proline-rich proteoglycan (PIR accession | 0.52 |
| | | number B48013); cDNA EST EMBL:D66054 comes from this gene; | |
| | | cDNA EST EMBL:D69700 comes from this gene; cDNA EST | |
| | | yk446b10.3 comes from this gene; cDNA EST yk446b10... | |
| 312 | 117589 | CIRCUMSPOROZOITE PROTEIN PRECURSOR precursor - | 9e−005 |
| | | Plasmodium knowlesi (strain Nuri) >gi\|160198 | |
| 313 | 2781381 | (AC004013) Similar to rabbit A-kinase-anchoring protein sapiens] | 1e−019 |
| 317 | 730269 | PAN1 PROTEIN >gi\|626783\|pir\|\|S48440 poly(A)-specific | 7.2 |
| | | ribonuclease (EC 3.1.13.4) - yeast PAN1_YEAST P32521 PAB- | |
| | | DEPENDENT POLY(A)-SPECIFIC RIBONONUCLEASE but | |
| 318 | 3201610 | (AC004669) unknown protein [Arabidopsis thaliana] | 5.4 |
| 319 | 3129975 | (AL023516) Complement C4 [Gallus gallus] | 2.4 |
| 322 | 1850592 | (U88295) carnitine palmitoyltransferase II [Rattus norvegicus] | 6.9 |
| 323 | 3881650 | (Z70757) Weak similarity to the XFIN protein (Swiss Prot accession | 6.6 |
| | | number P08045) | |
| 324 | 2653998 | (AF032884) tnsC [Thiobacillus ferrooxidans] | 8.2 |
| 326 | 145111 | (M18083) periplasmic [NiFe] hydrogenase protein small subunit | 0.37 |
| | | precursor [Desulfovibrio gigas] | |
| 327 | 2244839 | (Z97337) hypothetical protein [Arabidopsis thaliana] | 3.1 |
| 328 | 3935180 | (AC004557) F17L21.23 [Arabidopsis thaliana] | 4.6 |
| 332 | 3328899 | (AE001320) hypothetical protein [Chlamydia trachomatis] | 7.4 |
| 333 | 4096264 | (U26528) Ig heavy chain [Oryctolagus cuniculus] | 7.1 |
| 334 | 3023209 | 168 KD SURFACE-LAYER PROTEIN PRECURSOR | 5.8 |
| | | [CONTAINS: 120 KD SURFACE-EXPOSED PROTEIN | |
| | | MEMBRANE PROTEIN OMPB); 32 KD BETA PEPTIDE] | |
| | | precursor - Rickettsia typhi >gi\|1871223 (L04661) crystalline surface | |
| 335 | 4589400 | (AB009958) polyprotein [satsuma dwarf virus] | 5.7 |
| 336 | 1709356 | SODIUM-DEPENDENT NORADRENALINE TRANSPORTER | 5.4 |
| | | (NOREPINEPHRINE TRANSPORTER) (NET) bovine | |
| | | >gi\|1050439\|emb\|CAA55645\|(X79015) norepinephrine transporter | |
| 337 | 4512671 | (AC006931) unknown protein [Arabidopsis thaliana] | 1.9 |
| 343 | 3757516 | (AC005167) putative TMV resistance protein [Arabidopsis thaliana] | 3.8 |
| 344 | 586480 | HYPOTHETICAL 62.6 KD PROTEIN IN CDS1-RPL2 | 0.26 |
| | | INTERGENIC REGION >gi\|626496\|pir\|\|S45886 hypothetical protein | |
| | | YBR030w - yeast (Saccharomyces cerevisiae) | |
| 345 | 4239381 | 88K E-26-specific domain protein Pok - Drosophila | 0.054 |
| | | >gi\|217342\|dbj\|BAA01080\|(D10228) Ets domain protein | |
| 347 | 1653153 | (D90911) acriflavin resistance protein | 5.5 |
| 348 | 2147342 | E4 protein - human papillomavirus type 14D | 0.67 |
| 349 | 3914412 | GENOME POLYPROTEIN [CONTAINS: N-TERMINAL | 0.95 |
| | | PROTEIN (P1); HELPER COMPONENT PROTEINASE | |
| | | INCLUSION PROTEIN (CI); 6 KD PROTEIN 2 (6K2); GENOME- | |
| | | LINKED PROTEIN (VPG); NUCLEA... mosaic virus] | |
| 350 | 2997741 | (AF054838) tetraspan TM4SF; Tspan-1 [Homo sapiens] | 1e−017 |
| 351 | 1086900 | (U41278) contains similarity to G beta repeats | 3e−027 |
| 372 | 129036 | 2-OXOGLUTARATE DEHYDROGENASE E1 COMPONENT | 9.8 |
| | | (ALPHA-KETOGLUTARATE DEHYDROGENASE) | |
| | | dehydrogenase [Azotobacter vinelandii] | |
| 373 | 2120777 | cellulose synthase - Agrobacterium tumefaciens >gi\|710493 (L38609) | 9.7 |
| | | cellulose synthase celA gene [Agrobacterium tumefaciens] | |
| 374 | 628527 | hypothetical protein - Pseudomonas syringae syringae] | 9.6 |
| 375 | 123395 | HOMEOBOX PROTEIN NK-1 (S59/2) fly (Drosophila | 7.8 |
| | | melanogaster) >gi\|8531\|emb\|CAA390671 sp.] | |
| 376 | 3722000 | (AF035323) survival motor neuron protein | 7.8 |
| 377 | 3881030 | (AL021493) Y51A2B.2 [Caenorhabditis elegans] | 7.4 |
| 378 | 4505777 | PHD finger protein 1 >gi\|2660720 1) [Homo sapiens] | 6 |
| 379 | 2190501 | (X53706) immunoglobulin alpha-2 heavy chain [Pan troglodytes] | 5.8 |
| 380 | 2688643 | (AE001171) conserved hypothetical integral membrane protein | 5.7 |
| | | [Borrelia burgdorferi] | |
| 381 | 1086832 | (U41264) coded for by C. elegans cDNA cm13g1; Similar to | 5.4 |
| | | bumetanide-sensitive Na-K-Cl cotransporter. | |
| 382 | 1931639 | (U95973) lysophospholipase isolog [Arabidopsis thaliana] | 4.6 |
| 383 | 3757516 | (AC005167) putative TMV resistance protein [Arabidopsis thaliana] | 4.5 |
| 384 | 4504567 | interferon consensus sequence binding protein 1 >gi\|2275153 | 4.4 |
| | | (M91196) DNA-binding protein [Homo sapiens] | |
| 385 | 464822 | SUR1 PROTEIN >gi\|542362\|pir\|\|S41798 SUR1 protein - yeast | 4.4 |
| | | (Saccharomyces cerevisiae) cerevisiae] >gi\|976268\|dbj\|BAA05628\| | |
| 386 | 3023738 | EXOSTOSIN-L (MULTIPLE EXOSTOSIS-LIKE PROTEIN) | 4.3 |
| | | >gi\|1524413 (U67191) multiple exostosis-like protein [Homo | |
| 387 | 3878603 | (Z83116) M01B2.3 [Caenorhabditis elegans] | 3.4 |
| 388 | 3745858 | (L33180) BRO-a [Bombyx mori nuclear polyhedrosis virus] | 3.3 |
| 389 | 4321758 | (AF060669) polyprotein [Hepatitis E virus] | 3.3 |
| 390 | 2661037 | (AF035285) dihydroxyacetone phosphate acyltransferase | 3.3 |
| 391 | 2950355 | (AJ223300) homebox protein DRx [Drosophila melanogaster] | 2.7 |
| 392 | 3821973 | (AF061140) merozoite surface protein 1 [Plasmodium falciparum] | 2.5 |
| 393 | 1552187 | (D84375) ORF3 [Oryzias latipes] | 2 |
| 394 | 2493417 | S100 CALCIUM-BINDING PROTEIN A13 calcium-binding protein | 1.6 |
| | | A13 [Mus musculus] | |
| 395 | 3550082 | (AF071186) WW domain binding protein 11 [Mus musculus] | 1.5 |
| 396 | 1082665 | oligodendrocyte-specific proline-rich protein 2 - human | 1.5 |
| | | >gi\|1408050\|dbj\|BAA05660\|(D28114) MOBP [Homo sapiens] | |
| 397 | 3874925 | (Z68296) Similarity to Mouse A-RAF proto-oncogene | 1.2 |
| | | serine/threonine-protein kinase gene; cDNA EST EMBL:T01018 | |
| | | comes from this gene; cDNA EST EMBL:D33256 comes from this | |
| 398 | 2500886 | PROBABLE SIGNAL RECOGNITION 54 KD PROTEIN (SRP54) | 1.2 |
| | | >gi\|2129283\|pir\|\|E64312 signal recognition particle protein - | |
| | | Methanococcus jannaschii subunit SRP54 [Methanococcus | |
| 399 | 2291241 | (AF016428) contains similarity to Vaccinia virus 37 kd envelope | 1.1 |
| | | protein [Caenorhabditis elegans] | |
| 400 | 309958 | (L06798) class D tetracycline/H+ antiporter [Plasmid pRA1] | 0.91 |
| | | >gi\|575937\|dbj\|BAA03719\|(D16172) PP-TETA protein | |
| 401 | 3877379 | (Z46267) F49E2.2 [Caenorhabditis elegans] | 0.39 |
| 402 | 4539280 | (AL049498) putative transcription factor | 0.29 |
| 403 | 3293235 | (U96413) putative opine synthase [Agrobacterium tumefaciens] | 0.097 |
| 404 | 3329636 | (AF078786) No definition line found [Caenorhabditis elegans] | 0.003 |
| 405 | 3947614 | (AL023828) cDNA EST yk491f8.5 comes from this gene | 8e−019 |
| | | [Caenorhabditis elegans] | |
| 406 | 3947614 | (AL023828) cDNA EST yk491f8.5 comes from this gene | 1e−019 |
| | | [Caenorhabditis elegans] | |
| 407 | 3880930 | (AL021481) similar to Phosphoglucomutase and | 1e−022 |
| | | phosphomannomutase phosphoserine; cDNA EST EMBL:D36168 | |
| | | comes from this gene; cDNA EST EMBL:D70697 comes from this | |
| | | gene; cDNA EST yk373h9.5 comes from this gene; cDNA EST | |
| 408 | 3184082 | (AL023781) N-terminal acetyltransferase 1 | 1e−028 |
| 409 | 2500558 | PUTATIVE RIBONUCLEASE III (RNASE III) | 7e−030 |
| | | >gi\|3876420\|emb\|CAB03005\|(Z81070) similar to ribonuclease | |
| 410 | 3877493 | (Z48583) similar to ATPases associated with various cellular | 5e−044 |
| | | activities (AAA); cDNA EST EMBL:Z14623 comes from this gene; | |
| | | cDNA EST EMBL:D75090 comes from this gene; cDNA EST | |
| | | EMBL:D72255 comes from this gene; cDNA EST yk200e4.5... | |
| 411 | 3298605 | (AF057365) UDP N-acetylglucosamine transporter [Canis familiaris] | 1e−051 |
| 412 | 4103604 | (AF026031) putative mitochondrial outer membrane protein import | 1e−059 |
| | | receptor [Homo sapiens] | |
| 413 | 2662165 | (AB007902) HH0712 cDNA clone for KIAA0442 has a 574-bp | 4e−070 |
| | | insertion at position 1474 of the sequence of KIAA0442. [Homo | |
| 414 | 3882145 | (AB018255) KIAA0712 protein [Homo sapiens] | 9e−072 |
| 477 | 117552 | CHAPERONE PROTEIN CS3-1 PRECURSOR Escherichia coli | 9.3 |
| | | >gi\|41156\|emb\|CAA34815\|(X16944) 27 kD protein (AA 1 to 241) | |
| | | [Escherichia coli] | |
| 478 | 2983622 | (AE000726) hypothetical protein [Aquifex aeolicus] | 9.3 |
| 479 | 2088694 | (AF003135) W03F11.1 gene product [Caenorhabditis elegans] | 9.3 |
| 480 | 4240237 | (AB020681) KIAA0874 protein [Homo sapiens] | 9.2 |
| 481 | 1864073 | (U63002) T-cell receptor beta chain [Callithrix jacchus] | 9 |
| 482 | 2147334 | DNA helicase II 70K chain homolog - Rhipicephalus appendiculatus | 8.9 |
| | | >gi\|1063592 (L41356) ku autoantigen p70 homologue [Rhipicephalus | |
| | | appendiculatus] | |
| 483 | 3323145 | (AE001253) T. pallidum predicted coding region TP0827 | 8.7 |
| 484 | 2183251 | (AF002227) putative polyprotein [border disease virus strain C413] | 8.6 |
| 485 | 3875768 | (Z92830) cDNA EST yk223c7.5 comes from this gene; cDNA EST | 8.6 |
| | | yk307b2.5 comes from this gene; cDNA EST yk377h2.5 comes from | |
| | | this gene; cDNA EST yk223c7.3 comes from this gene; cDNA EST | |
| | | yk307b2.3 comes from this gene [Caenorhabditi... | |
| 486 | 1351589 | HYPOTHETICAL PROTEIN MG456 Mycoplasma genitalium | 8.4 |
| | | (SGC3) >gi\|1046175 (U39732) M. genitalium predicted coding | |
| | | region MG456 [Mycoplasma genitalium] >gi\|3845050 (U39727) | |
| | | conserved hypothetical protein [Mycoplasma genitalium] | |
| 487 | 3023800 | GLUCOSE-6-PHOSPHATASE (G6PASE) familiaris] | 6.9 |
| 488 | 450722 | (X71982) ORF j11R [African swine fever virus] | 6.8 |
| 489 | 2633756 | (Z99111) similar to heavy metal-transporting ATPase [Bacillus | 6.6 |
| 490 | 4049887 | (AF063866) ORF MSV024 ALI motif gene family protein | 6.5 |
| 491 | 1363994 | abdominal-A homeodomain protein - Junonia coenia >gi\|797277 | 5.3 |
| | | (L41931) abdominal-A homeodomain protein [Junonia coenia] | |
| 492 | 2370493 | (Z98944) hypothetical protein | 5.3 |
| 493 | 140631 | HYPOTHETICAL 25.9 KD PROTEIN FP25.9 [Fowlpox virus] | 5.3 |
| 494 | 400927 | RIBONUCLEOPROTEIN RB97D ribonucleoprotein [Drosophila | 5.1 |
| 495 | 2462935 | (Y12321) open reading frame 1 [Brassica oleracea] | 5 |
| 496 | 2160189 | (AC000132) Similar to A. thaliana receptor-like protein kinase | 5 |
| | | (gb\|RLK5_ARATH). ESTs gb\|ATTS0475, gb\|ATTS4362 come from | |
| | | this gene. [Arabidopsis thaliana] | |
| 497 | 1850972 | (U84144) putative fimbrial chaperone [Escherichia coli] | 4.9 |
| 498 | 2145678 | B1549_C3_230 protein - Mycobacterium leprae | 4.2 |
| 499 | 3182952 | CGMP-INHIBITED 3′, 5′-CYCLIC PHOSPHODIESTERASE A | 4.2 |
| | | (CYCLIC GMP INHIBITED PHOSPHODIESTERASE A) (CGI- | |
| | | PDE A) >gi\|1145304 (U38179) cyclic nucleotide phosphodiesterase | |
| 500 | 4493738 | (AL034358) predicted using hexExon; L4830.1, Hypothetical | 4.2 |
| | | protein, len: 1107 [Leishmania major] | |
| 501 | 730336 | PUTATIVE POLYKETIDE SYNTHASE PKSL 2.3.1.-) - Bacillus | 4.2 |
| | | subtilis >gi\|40058\|emb\|CAA78479\|subtilis] >gi\|528996 (U11039) | 4.2 |
| | | polyketide synthase polyketide synthase of type I [Bacillus subtilis] | |
| 502 | 188908 | (M19720) L-myc protein [Homo sapiens] | 4 |
| 503 | 4056436 | (AC005990) EST gb\|AA650912 comes from this gene. | 3.9 |
| 504 | 3874349 | (Z81035) predicted using Genefinder; Similarity to Sheep | 3.9 |
| | | vasopressin V1A receptor (SW:P48043) | |
| 505 | 3874067 | (Z93374) similar to 7TM receptor elegans] | 3.9 |
| 506 | 3979818 | (Z49967) cDNA EST EMBL:T00743 comes from this gene; cDNA | 3.2 |
| | | EST EMBL:D69356 comes from this gene; cDNA EST | |
| | | EMBL:D65790 comes from this gene; cDNA EST EMBL:D70463 | |
| | | comes from this gene; cDNA EST EMBL:D66620 comes from this | |
| 507 | 119462 | ENVELOPE POLYPROTEIN GP160 PRECURSOR 2] | 3.2 |
| | | >gi\|225570\|prf\|\|1306388H gene env [Human immunodeficiency virus | |
| 508 | 117657 | COLICIN V PRODUCTION PROTEIN (DEDE PROTEIN) (PUR | 3.1 |
| | | REGULON 18 KD PROTEIN) colicin V production [Escherichia | |
| | | coli] dedE protein [Escherichia coli] | |
| 509 | 114441 | ATP SYNTHASE A CHAIN (PROTEIN 6) 3.6.1.34) protein 6 - fruit | 3 |
| | | fly (Drosophila yakuba) mitochondrion (SGC4) | |
| | | >gi\|12921\|emb\|CAA25442\|(X00924) ATPase subunit 6 [Drosophila | |
| 510 | 1280094 | (U55369) No definition line found [Caenorhabditis elegans] | 3 |
| 511 | 226131 | thyroid hormone receptor alpha 2 [Rattus norvegicus] | 3 |
| 512 | 3860855 | (AJ235271) GUANOSINE PENTAPHOSPHATE | 2.9 |
| | | PHOSPHOHYDROLASE (gppA) [Rickettsia prowazekii] | |
| 513 | 584834 | CELLULOSE SYNTHASE OPERON C PROTEIN xylinus] | 2.4 |
| | | >gi\|1090660\|prf\|\|2019362C acsC gene | |
| 514 | 629777 | chitinase (EC 3.2.1.14) - barley vulgare] | 2.2 |
| 515 | 2555183 | (AF026504) SPA-1 like protein p1294 [Rattus norvegicus] | 2.2 |
| 516 | 4503737 | forkhead (Drosophila) homolog 1 HEAD DOMAIN PROTEIN | 1.8 |
| | | FKHR >gi\|631145\|pir\|\|S40521 FKHR protein - human >gi\|435423 | |
| | | (U02310) fork head domain protein [Homo sapiens] | |
| | | >gi\|737918\|prf\|\|1923399A FKHR gene [Homo sapiens] | |
| 517 | 3790719 | (AF099916) contains similarity to C2H2-type zinc fingers | 1.8 |
| 518 | 1785942 | (U83412) CAG [Drosophila melanogaster] | 1.8 |
| 519 | 3877036 | (Z81079) predicted using Genefinder; similar to collagen; cDNA | 1.4 |
| | | EST EMBL:M88890 comes from this gene; cDNA EST | |
| | | EMBL:Z14325 comes from this gene; cDNA EST EMBL:D27520 | |
| | | comes from this gene; cDNA EST EMBL:D72240 comes from this | |
| 520 | 281654 | hypothetical protein 24 - Agrobacterium tumefaciens plasmid | 1.3 |
| | | pTi15955 >gi\|39086\|emb\|CAA25186\|tumefaciens] | |
| 521 | 114972 | BETA-GLUCOSIDASE (GENTIOBIASE) Ruminococcus albus | 1.3 |
| | | >gi\|45968\|emb\|CAA33461\|(X15415) beta-glucosidase (AA 1-947) | |
| | | [Ruminococcus albus] albus] | |
| 522 | 1707085 | (U80451) Similar to collagen [Caenorhabditis elegans] | 1.3 |
| 523 | 483163 | nonstructural protein - hepatitis E virus RNA-directed RNA | 1.3 |
| | | polymerase [Hepatitis E virus] | |
| 524 | 4455275 | (AL035527) putative protein [Arabidopsis thaliana] | 0.99 |
| 525 | 4376875 | (AE001642) CT465 hypothetical protein | 0.45 |
| 526 | 2494911 | HYPOTHETICAL PROTEIN KIAA0124 product is novel. [Homo | 0.44 |
| 527 | 4539280 | (AL049498) putative transcription factor | 0.28 |
| 528 | 1842255 | (U74613) hepatocyte nuclear factor-3/fork head homolog 11B [Homo | 0.28 |
| 529 | 231977 | D(4) DOPAMINE RECEPTOR (D(2C) DOPAMINE RECEPTOR) | 0.073 |
| | | >gi\|203916 (M84009) dopamine receptor D4 | |
| 530 | 987050 | (X65335) lacZ [Cloning vector pSV-beta-Galactosidase Control] | 0.042 |
| 531 | 2493416 | S100 CALCIUM-BINDING PROTEIN A13 calcium-binding protein | 0.031 |
| | | A13 (S100A13) [Homo sapiens] | |
| 532 | 1778844 | (U83086) LimA [Dictyostelium discoideum] | 0.0006 |
| 533 | 3288470 | (AJ224360) surf5c [Homo sapiens] | 5e−015 |
| 534 | 3947614 | (AL023828) cDNA EST yk491f8.5 comes from this gene | 1e−015 |
| | | [Caenorhabditis elegans] | |
| 535 | 1086860 | (U41272) Similar to man(9)-alpha-mannosidase. | 3e−028 |
| 536 | 3875451 | (Z66496) cDNA EST EMBL:D71941 comes from this gene; cDNA | 2e−030 |
| | | EST EMBL:D74691 comes from this gene; cDNA EST | |
| | | EMBL:D76330 comes from this gene; cDNA EST EMBL:D65192 | |
| | | comes from this gene; cDNA EST EMBL:D68540 comes from this | |
| 537 | 3877493 | (Z48583) similar to ATPases associated with various cellular | 1e−035 |
| | | activities (AAA); cDNA EST EMBL:Z14623 comes from this gene; | |
| | | cDNA EST EMBL:D75090 comes from this gene; cDNA EST | |
| | | EMBL:D72255 comes from this gene; cDNA EST yk200e4.5... | |
| 538 | 3169010 | (AJ006412) putative GTP-binding protein | 2e−042 |
| 539 | 3877493 | (Z48583) similar to ATPases associated with various cellular | 4e−044 |
| | | activities (AAA); cDNA EST EMBL:Z14623 comes from this gene; | |
| | | cDNA EST EMBL:D75090 comes from this gene; cDNA EST | |
| | | EMBL:D72255 comes from this gene; cDNA EST yk200e4.5... | |
| 540 | 3877493 | (Z48583) similar to ATPases associated with various cellular | 3e−044 |
| | | activities (AAA); cDNA EST EMBL:Z14623 comes from this gene; | |
| | | cDNA EST EMBL:D75090 comes from this gene; cDNA EST | |
| | | EMBL:D72255 comes from this gene; cDNA EST yk200e4.5... | |
| 608 | 3882189 | (AB018277) KIAA0734 protein [Homo sapiens] | 9.9 |
| 609 | 3877937 | (Z48716) similarity to a transmembranous region of ubiquinol- | 9.6 |
| | | cytochrome-C reductase (PIR accession number S38960); cDNA | |
| | | EST EMBL:T00461 comes from this gene; cDNA EST | |
| | | EMBL:D27071 comes from this gene; cDNA EST EMBL:D27070 | |
| 610 | 3643019 | (AF064703) glucose transporter 1; CeGT1 [Drosophila | 8.4 |
| 611 | 3219946 | HYPOTHETICAL PROTEIN MJ1394 Methanococcus jannaschii | 8 |
| | | >gi\|1592041 (U67579) conserved hypothetical protein | |
| 612 | 3219946 | HYPOTHETICAL PROTEIN MJ1394 Methanococcus jannaschii | 8 |
| | | >gi\|1592041 (U67579) conserved hypothetical protein | |
| 613 | 2833328 | FIBRILLARIN | 7.9 |
| 614 | 4505481 | nucleoporin 88 kD complex protein [Homo sapiens] | 7.8 |
| 615 | 220578 | (D00570) open reading frame (251 AA) [Mus musculus] | 7.8 |
| 616 | 266810 | NAD(P) TRANSHYDROGENASE SUBUNIT BETA | 7.6 |
| | | transhydrogenase [Escherichia coli] transhydrogenase (B-specific) | |
| | | (EC 1.6.1.1) b chain NAD(P)+transhydrogenase (B-specific) (EC | |
| | | (1.6.1.1) b chain [Escherichia coli] >gi\|1787886 (AE000255) pyridine | |
| 617 | 807646 | (M17294) unknown protein [Human herpesvirus 4] | 7.6 |
| 618 | 829186 | (X03879) rudimentary protein fragment | 7.4 |
| 619 | 4322346 | (AF081825) sodium-dependent high-affinity dicarboxylate | 7.4 |
| | | transporter [Rattus norvegicus] | |
| 620 | 3334785 | (AL031107) hypothetical protein SC5A7.04c | 7.4 |
| 621 | 1346720 | PHOSPHATIDYLINOSITOL-4-PHOSPHATE 5-KINASE TYPE II | 7.3 |
| | | ALPHA (PIP5KII-ALPHA) KINASE) >gi\|1079474\|pir\|\|A55967 1- | |
| | | phosphatidylinositol-4-phosphate 5-kinase (EC 2.7.1.68) - human | |
| | | >gi\|758697 (U14957) 53 K isoform of Type II phosphatidylinositol-4- | |
| 622 | 4105819 | (AF050175) Rab7 [Homo sapiens] | 6.4 |
| 623 | 155865 | (M93125) 80 kDa protein [Babesia bovis] | 6.3 |
| 624 | 2133638 | boule protein - fruit fly (Drosophila melanogaster) >gi\|1395211 | 6.2 |
| | | (U51858) boule protein | |
| 625 | 1788052 | (AE000270) putative transport system permease protein | 6.2 |
| 626 | 3875616 | (Z77657) F08H9.9 [Caenorhabditis elegans] | 6.2 |
| 627 | 2499150 | HYPOTHETICAL PROTEIN IN CPS REGION | 6.2 |
| 628 | 1170758 | GALECTIN-3 (GALACTOSE-SPECIFIC LECTIN 3) (MAC-2 | 6.1 |
| | | ANTIGEN) (IGE-BINDING PROTEIN) (35 KD LECTIN) | |
| | | (CARBOHYDRATE BINDING PROTEIN 35) (CBP 35) | |
| 629 | 2495335 | HEAT SHOCK PROTEIN 42 (42 KD HEAT SHOCK PROTEIN) | 6 |
| | | >gi\|1077219\|pir\|\|S49767 heat shock protein HSP42 - yeast | |
| | | (Saccharomyces cerevisiae) | |
| 630 | 1086677 | (U41020) coded for by C. elegans cDNA yk64f5.3; coded for by C. | 6 |
| | | elegans cDNA yk64f5.5; Similar to zinc finger. [Caenorhabditis | |
| 631 | 1170758 | GALECTIN-3 (GALACTOSE-SPECIFIC LECTIN 3) (MAC-2 | 6 |
| | | ANTIGEN) (IGE-BINDING PROTEIN) (35 KD LECTIN) | |
| | | (CARBOHYDRATE BINDING PROTEIN 35) (CBP 35) | |
| 632 | 440957 | Achaete-Scute homolog Mash-1 gene product | 6 |
| 633 | 1786037 | (U72284) NADH dehydrogenase subunit 2 [Apis mellifera] | 6 |
| 634 | 2495335 | HEAT SHOCK PROTEIN 42 (42 KD HEAT SHOCK PROTEIN) | 6 |
| | | >gi\|1077219\|pir\|\|S49767 heat shock protein HSP42 - yeast | |
| | | (Saccharomyces cerevisiae) | |
| 635 | 2662541 | (AF036687) contains similarity to protease inhibitors, WAP-type four- | 5.7 |
| | | disulfide core domains and thyroglobulin type-1 repeats | |
| 636 | 118249 | DAUGHTERLESS PROTEIN fly (Drosophila melanogaster) | 5.6 |
| | | >gi\|7839\|emb\|CAA68368\|melanogaster] >gi\|157174 (J03148) | |
| | | daughterless protein | |
| 637 | 2447066 | (U42580) A570L [Paramecium bursaria Chlorella virus 1] | 5.6 |
| 638 | 400927 | RIBONUCLEOPROTEIN RB97D ribonucleoprotein [Drosophila | 4.9 |
| 639 | 121189 | GLUCOSE INHIBITED DIVISION PROTEIN A gidA | 4.8 |
| 640 | 3033398 | (AC004238) putative phosphoribosylaminoimidazolecarboxamide | 4.6 |
| | | formyltransferase [Arabidopsis thaliana] | |
| 641 | 126691 | POSSIBLE MALTASE PRECURSOR (LARVAL VISCERAL | 4.5 |
| | | PROTEIN D) >gi\|103222\|pir\|\|S08597 hypothetical protein D - fruit | |
| | | fly (Drosophila melanogaster) melanogaster] | |
| 642 | 134087 | RETROTRANSPOSABLE ELEMENT SLACS 132 KD PROTEIN | 4.3 |
| | | (ORF2) >gi\|84054\|pir\|\|S14916 hypothetical protein 2 - Trypanosoma | |
| | | brucei gambiense transposon SLACS >gi\|10535\|emb\|CAA34931\| | |
| 643 | 567166 | (L03172) This CDS feature is included to show the translation of the | 3.7 |
| | | corresponding V_region. Presently translation qualifiers on V_region | |
| | | features are illegal | |
| 644 | 4355 | (Z14126) RhoNUC protein [Saccharomyces cerevisiae] | 3.7 |
| 645 | 462679 | MYOSIN IB HEAVY CHAIN heavy chain [Dictyostelium | 3.6 |
| 646 | 1098989 | (U41508) similar to C. elegans proteins C26E6.9A and C26E6.9B; | 2.1 |
| | | weakly similar to malate synthase G | |
| 647 | 3249559 | (AF018261) EH domain binding protein Epsin [Rattus norvegicus] | 2 |
| 648 | 2435594 | (AF026212) No definition line found [Caenorhabditis elegans] | 2 |
| 649 | 995808 | (U32240) Ig heavy chain [Mus musculus] | 2 |
| 650 | 3281870 | (AL031004) putative protein [Arabidopsis thaliana] | 1.8 |
| 651 | 728837 | !!!ALU SUBFAMILY SQ WARNING ENTRY | 1.6 |
| 652 | 1628461 | (Y08775) Men-3 [Silene latifolia] | 1.2 |
| 653 | 483165 | hypothetical protein - Marek's disease virus gammaherpesvirus | 0.93 |
| | | tumorigenicity associated mRNA, two complete cds's.], gene products | |
| | | [Gallid herpesvirus type 1] >gi\|299459\|bbs\|129316 (S58431) CD4 | |
| | | precursor homolog to CD4 and IgM heavy chain [Gallid herpesvir | |
| 654 | 483165 | hypothetical protein - Marek's disease virus gammaherpesvirus | 0.87 |
| | | tumorigenicity associated mRNA, two complete cds's.], gene products | |
| | | [Gallid herpesvirus type 1] >gi\|299459\|bbs\|129316 (S58431) CD4 | |
| | | precursor homolog to CD4 and IgM heavy chain [Gallid herpesvir | |
| 655 | 120359 | RECOMBINASE FLP PROTEIN | 0.71 |
| 656 | 1711563 | STERYL-SULFATASE PRECURSOR (STEROID SULFATASE) | 0.55 |
| | | (STERYL-SULFATE SULFOHYDROLASE) sulfatase [Mus | |
| 657 | 731849 | HYPOTHETICAL 13.5 KD PROTEIN IN MOB1-SGA1 | 0.55 |
| | | INTERGENIC REGION >gi\|626376\|pir\|\|S48473 probable membrane | |
| | | protein YIL100w - yeast (Saccharomyces cerevisiae) | |
| | | >gi\|558707\|emb\|CAA86281\|(Z38125) orf, len: 117, CAI: 0.08, better | |
| 658 | 1669674 | (X86819) Microtubule-associated protein 4 | 0.3 |
| 659 | 2493735 | SKD3 PROTEIN SKD3 [Mus musculus] | 0.25 |
| 660 | 110030 | homeotic protein Hox 5.1 - mouse | 0.19 |
| 661 | 1947160 | (AF000298) weak similarity to collagens; glycine- and proline-rich | 0.012 |
| | | [Caenorhabditis elegans] | |
| 662 | 4567275 | (AC006841) hypothetical protein [Arabidopsis thaliana] | 0.005 |
| 663 | 2677676 | (AC002467) DRA protein (down-regulated in adenoma); sulfate | 4e−056 |
| | | transporter; match to P40879 (PID:g729367) [Homo sapiens] | |
| 671 | 345474 | hypothetical protein 2 - Mediterranean fruit fly | 9.4 |
| | | >gi\|5977\|emb\|CAA49660\|(X70053) unknown [Ceratitis capitata] | |
| 672 | 4538951 | (AL049488) putative protein [Arabidopsis thaliana] | 9.1 |
| 673 | 1749646 | (D89219) unnamed protein product | 7 |
| 674 | 320302 | ORF X protein - human papillomavirus type 41 | 5.5 |
| 675 | 267288 | REPLICATION PROTEIN E1 papillomavirus (type 1) | 5.4 |
| | | >gi\|61013\|emb\|CAA44657\|(X62844) E1 [Pygmy chimpanzee | |
| 676 | 3581899 | (AL031543) hypothetical serine-rich protein [Schizosaccharomyces | 4.2 |
| 677 | 3881119 | (AL032653) predicted using Genefinder; cDNA EST EMBL:D36367 | 4.2 |
| | | comes from this gene; cDNA EST yk408c12.5 comes from this gene | |
| | | [Caenorhabditis elegans] | |
| 678 | 1169663 | TRANSCRIPTIONAL ACTIVATOR FE6S APP interacting protein | 4 |
| | | [Rattus rattus] | |
| 679 | 1565257 | (X57108) cerebroside sulfate activator | 3.2 |
| 680 | 3093358 | (AJ005559) SPR2A protein [Mus musculus] | 1.1 |
| 681 | 1763113 | (U71019) NADH dehydrogenase subunit F [Arrhenatherum elatius] | 0.6 |
| 682 | 3548791 | (AC005620) R33590_1 [Homo sapiens] | 0.46 |
| 683 | 4567275 | (AC006841) hypothetical protein [Arabidopsis thaliana] | 0.025 |
| 684 | 3882311 | (AB018338) KIAA0795 protein [Homo sapiens] | 7e−017 |
| 687 | 2443342 | (D88764) alpha 2 type I collagen [Rana catesbeiana] | 5.1 |
| 688 | 220464 | (D00232) E3 anti-[4-hydroxy-3-nitrophenyl(phenolate + phenolic | 6.4 |
| | | form)]acetyl mAb V-L region [Mus musculus] | |
| 689 | 1351502 | HYPOTHETICAL PROTEIN MG181 Mycoplasma genitalium | 2.1 |
| | | (SGC3) >gi\|3844777 (U39697) conserved hypothetical protein | |
| 693 | 250891 | (S39392) protein tyrosine phosphatase, PTPase | 4.6 |
| 695 | 3323042 | (AE001245) sugar ABC transporter, periplasmic binding protein | 5.8 |
| | | (msmE) [Treponema pallidum] | |
| 696 | 1001741 | (D64004) hypothetical protein | 2.7 |
| 697 | 1169288 | DIHYDROXYACETONE KINASE (GLYCERONE KINASE) | 3e−010 |
| | | >gi\|493083 (U09771) dihydroxyacetone kinase | |
| 698 | 1169288 | DIHYDROXYACETONE KINASE (GLYCERONE KINASE) | 3e−010 |
| | | >gi\|493083 (U09771) dihydroxyacetone kinase | |
| 699 | 3879530 | (Z49130) cDNA EST yk486b9.3 comes from this gene; cDNA EST | 7e−005 |
| | | yk486b9.5 comes from this gene | |
| 700 | 2191127 | (AF007269) A_IG002N01.1 gene product [Arabidopsis thaliana] | 0.0004 |
| 701 | 4504923 | keratin, hair, acidic, 2 type I intermediate filament [Homo sapiens] | 1.5 |
| 702 | 2633756 | (Z99111) similar to heavy metal-transporting ATPase [Bacillus | 7.4 |
| 703 | 3876796 | (Z81531) F36D3.6 [Caenorhabditis elegans] | 7.3 |
| 705 | 123299 | HOMEOBOX PROTEIN HOX-D4 (HOX-4B) protein Hox D4 - | 2.4 |
| | | human >gi\|296652\|emb\|CAA35237\|(X17360) hox 5.1 protein [Homo | |
| 706 | 99899 | DNA-directed RNA polymerase (EC 2.7.7.6) largest chain (isoform | 0.83 |
| | | C) - soybean (fragment) polymerase [Glycine max] | |
| 707 | 4263788 | (AC006068) hypothetical protein | 0.28 |
| 708 | 500858 | (D14168) 50 kDa lectin [Bombyx mori] | 4e−011 |
| 711 | 142283 | (M30318) put. periplasmic receptor protein (chvE); putative | 9.2 |
| | | [Agrobacterium tumefaciens] | |
| 712 | 4502949 | collagen, type II, alpha 1 congenital) | 6.9 |
| | | >gi\|115287\|sp\|P02458\|CA12_HUMAN PROCOLLAGEN ALPHA | |
| | | 1(II) CHAIN PRECURSOR [CONTAINS: CHONDROCALCIN]1- | |
| 713 | 4539163 | (AL049485) putative phytoene synthase | 0.81 |
| 715 | 4056437 | (AC005990) Strong similarity to PFAM PF\|00069 Eukaryotic protein | 2.3 |
| | | kinase domain. [Arabidopsis thaliana] | |
| 716 | 119296 | ELASTIN PRECURSOR (TROPOELASTIN) gallus] | 1.3 |
| 717 | 4226073 | (AF125443) contains similarity to S. pombe phosphatidyl synthase | 8e−016 |
| | | (GB:Z28295) [Caenorhabditis elegans] | |
| 719 | 2498761 | PEROXISOMAL MEMBRANE PROTEIN PMP30A protein - yeast | 1.3 |
| | | (Candida boidinii) >gi\|457391 (L27999) peroxisomal membrane | |
| | | protein 31 [Candida boidinii] | |
| 720 | 3914963 | SECRETORY CARRIER-ASSOCIATED MEMBRANE PROTEIN | 0.58 |
| | | 3 >gi\|2232237 (AF005036) secretory carrier membrane protein [Mus | |
| 721 | 4457204 | (AF108226) immunoglobulin mu heavy chain precursor | 0.15 |
| | | [Monodelphis domestica] | |
| 722 | 3860573 | (AJ235270) unknown [Rickettsia prowazekii] | 4.9 |
| 723 | 2829912 | (AC002291) SimilarATP-dependent RNA Helicase | 0.0002 |
| 724 | 2130573 | (U96771) putative polygalacturonase [Prevotella bryantii] | 6.1 |
| 725 | 539244 | hypothetical protein YKR028w - yeast | 6.1 |
| 726 | 2633502 | (Z99110) similar to hypothetical proteins from B. subtilis [Bacillus | 4.7 |
| 728 | 1078087 | hypothetical protein YLR424w - yeast | 1.6 |
| 729 | 4240219 | (AB020672) KIAA0865 protein [Homo sapiens] | 2 |
| 732 | 3165370 | (AB011874) alpha subunit of dinitrogenase reductase (Fe protein) | 9.3 |
| | | [unidentified nitrogen-fixing bacteria] | |
| 733 | 3882195 | (AB018280) KIAA0737 protein [Homo sapiens] | 2e−061 |
| 735 | 3859938 | (AB081101) reverse transcriptase [Lymantria dispar] | 2.3 |
| 737 | 974143 | (L42542) RLIP76 protein [Homo sapiens] | 8.4 |
| 738 | 3877493 | (Z48583) similar to ATPases associated with various cellular | 3e−047 |
| | | activities (AAA); cDNA EST EMBL:Z14623 comes from this gene; | |
| | | cDNA EST EMBL:D75090 comes from this gene; cDNA EST | |
| | | EMBL:D72255 comes from this gene; cDNA EST yk200e4.5... | |
| 739 | 4240235 | (AB020680) KIAA0873 protein [Homo sapiens] | 3e−052 |
| 740 | 4191810 | (AB006532) DNA helicase [Homo sapiens] | 1e−065 |
| 741 | 4507851 | reserved protease [Homo sapiens] | 2e−071 |
| 742 | 3876797 | (Z81531 cDNA EST EMBL:D66579 comes from this gene; cDNA | 2.7 |
| | | EST EMBL:D70408 comes from this gene; cDNA EST yk263d3.5 | |
| | | comes from this gene; cDNA EST yk275c1.5 comes from this gene; | |
| | | cDNA EST EMBL:C10270 comes from this gene [Caenorh... | |
| 743 | 4376875 | (AE001642) CT465 hypothetical protein | 0.53 |
| 744 | 91305 | sperm mitochondrial capsule selenoprotein - mouse | 0.32 |
| 745 | 1938549 | (U97016) similar to drosophila Rlc1 gene product ribosomal protein | 9e−016 |
| | | L4 (YML4) (NID:g459259) | |
| 751 | 3882189 | (AB018277) KIAA0734 protein [Homo sapiens] | 7.8 |
| 752 | 505665 | (U08023) novel cellular proto-oncogene [Homo sapiens] | 7.8 |
| 753 | 3170498 | (AF052872) APETALA3 homolog PcAP3 [Papaver californicum] | 6 |
| 754 | 1082243 | autotaxin precursor - human >gi\|537906 | 2.6 |
| 755 | 4507721 | titin >gi\|1212992\|emb\|CAA62188\|via Swiss-Prot; available at | 1.5 |
| | | present via e-mail from LABEIT@EMBL-Heidelberg.DE [Homo | |
| 756 | 3036883 | (AL022374) putative ABC transporter | 0.68 |
| 757 | 543593 | hypothetical 39.8 K protein (clone GV-B) - garlic virus B | 0.06 |
| 758 | 79960 | hypothetical 30.5 K protein - Enterococcus faecalis plasmid pAM-beta- | 1e−024 |
| | | 1 >gi\|3023044 (AF007787) orfC | |
| 759 | 3882195 | (AB018280) KIAA0737 protein [Homo sapiens] | 6e−060 |
| 766 | 1707719 | (Y08256) orf c02007 [Sulfolobus solfataricus] | 9.8 |
| 767 | 2133808 | immunoglobulin heavy chain - nurse shark | 7.6 |
| 768 | 1469880 | (D63483) The KIAA0149 gene product is related to Notch3. [Homo | 7.5 |
| 769 | 4454062 | (AJ132911) NorD protein [Bradyrhizobium japonicum] | 5.9 |
| 770 | 137532 | PROTEIN C2 >gi\|74386\|pir\|\|WZVZB6 59 K HindIII-C protein - | 4.4 |
| | | vaccinia virus (strain WR) | |
| 771 | 1098985 | (U41031) proline-rich [Caenorhabditis elegans] | 3.4 |
| 772 | 4378891 | (AF132481) Ese1L protein [Mus musculus] | 2.6 |
| 773 | 4557489 | cone-rod homeobox PROTEIN >gi\|2665534 (AF024711) cone rod | 1.5 |
| | | homeobox protein | |
| 774 | 2135894 | peripheral benzodiazepine receptor - human | 0.52 |
| 775 | 477495 | cell-fate determining gene Notch2 protein - rat | 0.51 |
| 776 | 1945493 | (U56965) Similar to NAD(P) transhydrogenase, mitochondrial; coded | 0.39 |
| | | for by C. elegans cDNA yk27c1.5; coded for by C. elegans cDNA | |
| | | yk35b9.5; coded for by C. elegans cDNA yk35b9.3; coded for by C. | |
| | | elegans cDNA yk161c9.3; coded for by C. elegans... | |
| 777 | 746516 | (U23517) D1022.7 [Caenorhabditis elegans] >gi\|3258651 elegans] | 0.059 |
| 778 | 2498786 | GLYCEROL-3-PHOSPHATE ACYLTRANSFERASE | 0.058 |
| | | PRECURSOR (GPAT) (P90) acyltransferase homolog - mouse | |
| | | >gi\|193367 (M77003) glycerol-3-phosphate acyltransferase [Mus | |
| 779 | 2134384 | procKr2 - chicken (fragment) gallus] | 0.015 |
| 780 | 4176500 | (AL031177) dJ889M15.3 (novel protein) | 0.001 |
| 781 | 500858 | (D14168) 50 kDa lectin [Bombyx mori] | 5e−010 |
| 782 | 4204294 | (AC003027) lcl\|prt_seq No definition line found | 3e−010 |
| 783 | 500858 | (D14168) 50 kDa lectin [Bombyx mori] | 2e−010 |
| 784 | 3184082 | (AL023781) N-terminal acetyltransferase 1 | 6e−014 |
| 785 | 3184082 | (AL023781) N-terminal acetyltransferase 1 | 3e−014 |
| 786 | 1330401 | (U58762) T27F7.1 gene product [Caenorhabditis elegans] | 6e−030 |
| 787 | 1330401 | (U58762) T27F7.1 gene product [Caenorhabditis elegans] | 3e−030 |
| 788 | 3879850 | (Z81592) predicted using Genefinder | 8e−034 |
| 789 | 1072198 | (U40942) No definition line found [Caenorhabditis elegans] | 1e−037 |
| 790 | 3327160 | (AB014573) KIAA0673 protein [Homo sapiens] | 1e−062 |
| 791 | 3413886 | (AB007931) KIAA0462 protein [Homo sapiens] | 1e−073 |
| 800 | 1903264 | (Y11824) hypothetical protein [Pisum sativum] | 9.5 |
| 801 | 2739276 | (AJ223176) Ser/Thr protein kinase | 5.6 |
| 802 | 4521264 | (AB015874) guanylate cyclase O1GC-R2 | 5.6 |
| 803 | 2144044 | zinc finger protein AT-BP2 - black rat protein AT-BP2 [Rattus rattus] | 5.5 |
| 804 | 4049682 | (AF063866) ORF MSV092 hypothetical protein [Melanoplus | 4.3 |
| | | sanguinipes entomopoxvirus] | |
| 805 | 2842704 | HYPOTHETICAL 73.3 KD PROTEIN C6G9.14 IN | 3.2 |
| | | CHROMOSOME I >gi\|1644326\|emb\|CAB03616.1\|protein | |
| 806 | 1526981 | (X99945) amino acid permease YeeF like protein [Salmonella | 2.5 |
| 807 | 1082243 | autotaxin precursor - human >gi\|537906 | 2.5 |
| 808 | 3660667 | (AF055079) inositol 1,4,5-trisphosphate receptor | 2.4 |
| 809 | 2500002 | PHOSPHORIBOSYLAMINE-GLYCINE LIGASE | 2.4 |
| | | PHOSPHORIBOSYLFORMYLGLYCINAMIDINE CYCLO- | |
| | | LIGASE (AIRS) synthetase, aminoimidazole ribonucleotide | |
| | | synthetase, glycinamide.ribonucleotide transformylase {EC 6.3.4.13, | |
| | | 6.3.3.1, 2.1.2.2} [Chironomus tentans, Peptide, 1371 aa | |
| 810 | 4507721 | titin >gi\|1212992\|emb\|CAA62188\|via Swiss-Prot; available at | |
| | | present via e-mail from LABEIT@EMBL-Heidelberg.DE [Homo | 1.5 |
| 811 | 4507721 | titin >gi\|1212992\|emb\|CAA62188\|via Swiss-Prot; available at | 1.4 |
| | | present via e-mail from LABEIT@EMBL-Heidelberg.DE [Homo | |
| 812 | 2833215 | INTERFERON-ACTIVATABLE PROTEIN 205 protein - mouse | 1.1 |
| | | >gi\|385703\|bbs\|133592 (S62227) D3=lipopolysaccharide-inducible | |
| | | [mice, macrophages, Peptide, 425 aa] [Mus sp.] | |
| 813 | 1262910 | (U51645) cytidine triphosphate synthetase [Plasmodium falciparum] | 0.64 |
| 814 | 1245061 | (U46069) fertilin alpha subunit [Oryctolagus cuniculus] | 0.005 |
| 815 | 4493746 | (AL034358) predicted using hexExon; L4830.10, Hypothetical | 0.003 |
| | | protein, len: 816 aa [Leishmania major] | |
| 816 | 3283350 | (AF062378) calmodulin-binding protein SHA1 [Mus musculus] | 0.003 |
| 817 | 4204294 | (AC003027) lcl\|prt_seq No definition line found | 3e−006 |
| 818 | 4309681 | (AC006930) R33423_1 [Homo sapiens] | 4e−007 |
| 819 | 3293547 | (AF072709) putative oxidoreductase [Streptomyces lividans] | 5e−013 |
| 820 | 1086900 | (U41278) contains similarity to G beta repeats | 2e−028 |
| 821 | 3878739 | (Z73428) similar to Zinc finger, C3HC4 type (RING finger); cDNA | 6e−031 |
| | | EST EMBL:D67323 comes from this gene [Caenorhabditis elegans] | |
| | | >gi\|3881096\|emb\|CAB16481\|finger); cDNA EST EMBL:D67323 | |
| | | comes from this gene | |
| 822 | 3882195 | (AB018280) KIAA0737 protein [Homo sapiens] | 1e−053 |
| 823 | 3095186 | (AF057140) cargo selection protein TIP47 [Homo sapiens] | 1e−060 |
| 841 | 1915885 | (Y08370) alpha-amylase [Crassostrea gigas] | 9.2 |
| 842 | 1621461 | (U73103) laccase [Liriodendron tulipifera] | 9.2 |
| 843 | 232620 | (S41487) possible ribosomal protein=1(3)S12 Canton S wild type | 9.1 |
| | | [Drosophila melanogaster, Peptide, 73 aa] [Drosophila melanogaster] | |
| 844 | 1731338 | HYPOTHETICAL 73.6 KD PROTEIN CY49.21 | 6.9 |
| | | >gi\|1370248\|emb\|CAA98194\|(Z73966) hypothetical protein Rv2082 | |
| 845 | 3873261 | (AF096295) cytochrome oxidase subunit I [Naja siamensis] | 6.9 |
| 846 | 4049727 | (AF063866) ORF MSV173 putative serine/threonine protein kinase | 5.5 |
| | | Swinepox virus C20L homolog (vaccinia F10L), similar to | |
| | | SW:P32216 [Melanoplus sanguinipes entomopoxvirus] | |
| 847 | 1175595 | HYPOTHETICAL PROTEIN HI1265 | 4.1 |
| 848 | 446631 | collagen:SUBUNIT=alpha2:ISOTYPE=IX [Homo sapiens] | 4.1 |
| 849 | 627171 | odz protein - fruit fly (Drosophila sp.) product=tenascin homolog | 3.2 |
| | | [Drosophila melanogaster, 9- to 12-hour-old embryos, Peptide, 2406 | |
| 850 | 3986440 | (AF076785) serum amyloid A-activating factor SAF-5 | 3.1 |
| 851 | 91312 | stem cell protein ERA-1-399, retinoic acid-induced - mouse | 1.4 |
| | | >gi\|387146 (M22115) ERA-1-399 protein [Mus musculus] | |
| 852 | 4102043 | (AF008203) homeobox protein [Homo sapiens] | 1.3 |
| 853 | 746516 | (U23517) D1022.7 [Caenorhabditis elegans] >gi\|3258651 elegans] | 0.28 |
| 854 | 2935691 | (AF032122) unknown [Streptococcus thermophilus bacteriophage | 0.21 |
| 855 | 99972 | nodule-specific (hydroxy) proline-rich protein | 0.014 |
| 856 | 1169288 | DIHYDROXYACETONE KINASE (GLYCERONE KINASE) | 1e−005 |
| | | >gi\|493083 (U09771) dihydroxyacetone kinase | |
| 857 | 3721912 | (AB017156) gob-5 [Mus musculus] | 1e−011 |
| 858 | 868241 | (U29488) C56C10.3 gene product [Caenorhabditis elegans] | 3e−012 |
| 859 | 3293547 | (AF072709) putative oxidoreductase [Streptomyces lividans] | 5e−013 |
| 860 | 2228750 | (U93868) RNA polymerase III subunit [Homo sapiens] | 1e−015 |
| 861 | 1175412 | HYPOTHETICAL 24.2 KD PROTEIN C13A11.03 IN | 5e−021 |
| | | CHROMOSOME I >gi\|984224\|emb\|CAA90804\| | |
| 883 | 4505413 | non-metastatic cells 5, protein expressed in (nucleoside-diphosphate | 9 |
| | | kinase) KINASE HOMOLOG 5 (NDK-H 5) (NDP KINASE | |
| | | HOMOLOG 5) nucleoside-diphosphate kinase [Homo sapiens] | |
| 884 | 3308984 | (AB008516) mtprd [Mus musculus] | 9 |
| 885 | 3273643 | (AF042021) olfactory receptor [Sus scrofa] | 9 |
| 886 | 1170338 | HETEROCHROMATIN PROTEIN 1 HOMOLOG ALPHA (HP1 | 9 |
| | | ALPHA) (ANTIGEN P25) >gi\|184311 (L07515) complete cds.], | |
| | | gene product [Homo sapiens] chromosomal autoantigen [human, | |
| 887 | 1125745 | (U42832) coded for by C. elegans cDNA yk107c8.5; coded for by C. | 6.9 |
| | | elegans cDNA yk107c8.3; Similar to UDP-glucuronosyltransferase. | |
| | | [Caenorhabditis elegans] | |
| 888 | 2921102 | (AF020337) P6 [rice ragged stunt virus] | 6.9 |
| 889 | 1707199 | (U80841) C13A10.1 gene product [Caenorhabditis elegans] | 6.8 |
| 890 | 1125745 | (U42832) coded for by C. elegans cDNA yk107c8.5; coded for by C. | 5.3 |
| | | elegans cDNA yk107c8.3; Similar to UDP-glucuronosyltransferase. | |
| | | [Caenorhabditis elegans] | |
| 891 | 2493564 | PUTATIVE RIBOFLAVIN BIOSYNTHESIS ENZYME | 5.2 |
| | | >gi\|1707704\|emb\|CAA69508\|(Y08256) riboflavin biosynthesis | |
| | | protein ribG [Sulfolobus solfataricus] | |
| 892 | 2276148 | (Z81463) Similarity to C.elegans zinc finger proteins [Caenorhabditis | 5.2 |
| 893 | 1346425 | L-LACTATE DEHYDROGENASE 1.1.1.27) - Mycoplasma | 5.2 |
| | | genitalium (SGC3) >gi\|1046180 | |
| 894 | 4234795 | (AF078135) unknown [Leptospira borgpetersenii] | 5.2 |
| 895 | 3874963 | (Z92780) cDNA EST EMBL:D75953 comes from this gene | 4 |
| | | [Caenorhabditis elegans] | |
| 896 | 3875723 | (Z54270) similar to membrane glycoprotein | 3.9 |
| 897 | 1086593 | (U41007) C33H5.15 gene product [Caenorhabditis elegans] | 3.9 |
| 898 | 3289979 | (AC005263) SP62_HUMAN; SAP 62; SF3A66 [Homo sapiens] | 3.1 |
| 899 | 2208965 | (Y10528) cyanide insensitive terminal oxidase [Pseudomonas | 3 |
| 900 | 4539386 | (AL035526) extensin-like protein | 1.8 |
| 901 | 2662561 | (AF036692) Similar to seven transmembrane receptor | 1.8 |
| 902 | 2129184 | pheromone shutdown protein homolog - Methanococcus jannaschii | 1.3 |
| | | >gi\|592009 (U67576) pheromone shutdown protein (traB) | |
| | | [Methanococcus jannaschii] | |
| 903 | 2317864 | (U78289) tylactone synthase module 7 [Streptomyces fradiae] | 0.61 |
| 904 | 3808242 | (AF069669) pol protein [Human immunodeficiency virus type 1] | 0.6 |
| 905 | 3327128 | (AB014557) KIAA0657 protein [Homo sapiens] | 0.46 |
| 906 | 294529 | (L14933) convertase PC5 [Rattus norvegicus] | 0.35 |
| 907 | 3005087 | (AF044924) hook2 protein [Homo sapiens] | 0.12 |
| 908 | 3297817 | (AL031032) putative protein [Arabidopsis thaliana] | 0.025 |
| 909 | 3941342 | (AF043250) mitochondrial outer membrane protein [Homo sapiens] | 0.01 |
| | | >gi\|3941347 (AF043253) mitochondrial outer membrane protein | |
| | | [Homo sapiens] >gi\|4105703\|gb\|AAD02504\| | |
| 910 | 480989 | finger protein rfp - mouse (fragment) | 5e−006 |
| 911 | 2842526 | (AL021746) hypothetical anaphase promoting factor component | 3e−018 |
| | | [Schizosaccharomyces pombe] anaphase promoting complex | |
| 912 | 4226073 | (AF125443) contains similarity to S. pombe phosphatidyl synthase | 4e−022 |
| | | (GB:Z28295) [Caenorhabditis elegans] | |
| 913 | 3551821 | (AF058803) mucin 4 [Homo sapiens] | 3e−038 |
| 914 | 3882195 | (AB018280) KIAA0737 protein [Homo sapiens] | 4e−051 |
| 940 | 279539 | RNA-directed RNA polymerase (EC 2.7.7.48) - Marburg virus (strain | 8.7 |
| | | Musoke) >gi\|332179 (M92834) L Protein [Marburg virus] | |
| 941 | 117865 | CYTOCHROME B >gi\|2144288\|pir\|\|H22848 ubiquinol-cytochrome- | 8.6 |
| | | c reductase (EC 1.10.2.2) cytochrome b - Sauroleishmania tarentolae | |
| 942 | 3764008 | (AL008970) predicted using hexExon; MAL3P4.10 (PFC0515c), | 8.6 |
| | | Hypothetical protein, len: 1237 aa | |
| 943 | 3649770 | (Z98547) predicted using hexExon; MAL3P3.11 (PFC0380w), Dual- | 6.5 |
| | | specificity protein phosphatase, len: 581 aa; Similarity to protein | |
| | | phosphatases. S.cerevisiae protein-tyrosine phosphatase YVH1 | |
| | | (SW:PVH1_YEAST) BLAST Score: 123, s... | |
| 944 | 1125745 | (U42832) coded for by C. elegans cDNA yk107c8.5; coded for by C. | 5.1 |
| | | elegans cDNA yk107c8.3; Similar to UDP-glucuronosyltransferase. | |
| | | [Caenorhabditis elegans] | |
| 945 | 2208965 | (Y10528) cyanide insensitive terminal oxidase [Pseudomonas | 5 |
| 946 | 2146997 | L-selectin precursor - rabbit >gi\|847788 | 5 |
| 947 | 135091 | ALANYL-TRNA SYNTHETASE (ALANINE-TRNA LIGASE) | 3.8 |
| | | (ALARS) >gi\|95227\|pir\|\|S16897 alanine-tRNA ligase (EC 6.1.1.7) - | |
| | | Rhizobium leguminosarum bv. viciae synthetase [Rhizobium | |
| 948 | 1171815 | NADH-UBIQUINONE OXIDOREDUCTASE CHAIN 4 | 3.8 |
| | | >gi\|102585\|pir\|\|S26021 NADH dehydrogenase mitochondrion | |
| | | (SGC4) >gi\|559495\|emb\|CAA38170\|(X54253) ND4 protein [Ascaris | |
| 949 | 2120034 | glycoprotein B homolog precursor - feline herpesvirus 1 | 1.7 |
| | | >gi\|261095\|bbs\|120003 (S49775) glycoprotein B homolog [feline | |
| | | herpesvirus type 1 FHV-1, Peptide, 948 aa] [Feline herpesvirus 1] | |
| 950 | 4507539 | UNKNOWN >gi\|2587054 (AF027204) putative tetraspan | 0.99 |
| | | transmembrane protein L6H [Homo sapiens] | |
| 951 | 417237 | CHEMOTAXIS LAFT PROTEIN parahaemolyticus >gi\|677909 | 0.26 |
| | | (U20541) LafT [Vibrio parahaemolyticus] >gi\|1518953 (U52957) | |
| 952 | 3798624 | (AF082100) FK506 polyketide synthase [Streptomyces sp. MA6548] | 0.12 |
| 953 | 4493746 | (AL034358) predicted using hexExon; L4830.10, Hypothetical | 0.003 |
| | | protein, len: 816 aa [Leishmania major] | |
| 954 | 1175412 | HYPOTHETICAL 24.2 KD PROTEIN C13A11.03 IN | 6e−014 |
| | | CHROMOSOME I >gi\|984224\|emb\|CAA90804\| | |
| 955 | 4240235 | (AB020680) KIAA0873 protein [Homo sapiens] | 8e−044 |
| 976 | 1350599 | RIBONUCLEOSIDE-DIPHOSPHATE REDUCTASE ALPHA | 8.2 |
| | | CHAIN (RIBONUCLEOTIDE REDUCTASE) reductase (EC | |
| | | 1.17.4.1) - Mycoplasma genitalium (SGC3) reductase, alpha chain | |
| 977 | 731840 | HYPOTHETICAL 23.9 KD PROTEIN IN SGA1-KTR7 | 6.4 |
| | | INTERGENIC REGION >gi\|1077785\|pir\|\|S49791 probable | |
| | | membrane protein YIL089w - yeast (Saccharomyces cerevisiae) | |
| | | >gi\|577125\|emb\|CAA86705.1\|(Z46728) YI9910.07, unknown orf, | |
| 978 | 2132436 | probable membrane protein YDL118w - yeast | 6.3 |
| 979 | 1710518 | 60S RIBOSOMAL PROTEIN L22 homologue to human L22 | 6.3 |
| 980 | 3602956 | (AF041468) rbcR homolog [Guillardia theta] | 4.9 |
| 981 | 2146997 | L-selectin precursor - rabbit >gi\|847788 | 4.8 |
| 982 | 3880890 | (AL023838) predicted using Genefinder; similar to Helix-hairpin- | 4.8 |
| | | helix motif.; cDNA EST yk241d12.5 comes from this gene; cDNA | |
| | | EST yk401c3.5 comes from this gene; cDNA EST CEMSE47F | |
| | | comes from this gene [Caenorhabditis elegans] | |
| 983 | 3880727 | (AL032632) predicted using Genefinder | 2.8 |
| 984 | 1352547 | NADH-UBIQUINONE OXIDOREDUCTASE CHAIN 2 | 2.2 |
| | | >gi\|2147757\|pir\|\|S62766 NADH dehydrogenase caldarium) | |
| | | mitochondrion (fragment) subunit 2 [Cyanidium caldarium] | |
| 985 | 3551845 | (AF071879) capsid protein [Porcine circovirus] | 2.1 |
| 986 | 3249009 | (AF068139) S5 ribosomal protein/maturase fusion protein | 2.1 |
| | | [Cryphonectria parasitica] | |
| 987 | 1707126 | (U80454) T16A1.2 [Caenorhabditis elegans] | 2.1 |
| 988 | 1497971 | (U55797) VP6 [Bluetongue virus] | 1.7 |
| 989 | 3877568 | (Z70208) similar to collagen | 0.97 |
| 990 | 3309543 | (AF036382) MLL [Fugu rubripes] | 1e−005 |
| 991 | 4240235 | (AB020680) KIAA0873 protein [Homo sapiens] | 2e−039 |
| 1015 | 3257605 | (AP000005) 484aa long hypothetical protein | 8.2 |
| 1016 | 37593 | (X57019) unnamed protein product [Homo sapiens] | 8.1 |
| | | >gi\|238775\|bbs\|65126 (S65125) putative tyrosine kinase | |
| | | receptor=UFO [human, NIH3T3, Peptide, 894 aa] | |
| 1017 | 746533 | (U23520) similar to cuticular collagen [Caenorhabditis elegans] | 8 |
| 1018 | 729045 | BETA CASEIN PRECURSOR scrofa] | 7.9 |
| 1019 | 1170463 | INTERLEUKIN-1 BETA CONVERTASE PRECURSOR (IL-1BC) | 7.8 |
| | | (IL-1 BETA CONVERTING ENZYME) (ICE) converting enzyme | |
| 1020 | 1197641 | (U46859) DdhB [Yersinia enterocolitica (type 0:8)] | 6 |
| 1021 | 2104691 | (U92794) alpha glucosidase II, beta subunit [Mus musculus] | 4.7 |
| 1022 | 1139577 | (D63706) Orf5 [Streptomyces griseus] | 4.7 |
| 1023 | 136785 | EARLY GLYCOPROTEIN GP48 PRECURSOR glycoprotein (18) | 3.5 |
| | | [human herpesvirus 5] | |
| 1024 | 1314757 | (U54761) phosphoglucose isomerase [Erwinia amylovora] | 2.6 |
| 1025 | 2497383 | PUTATIVE TRANSPOSASE FOR INSERTION SEQUENCE IS408 | 1.6 |
| | | >gi\|309869 (L09108) IS408 transposase; putative [Pseudomonas | |
| | | cepacia] >gi\|1097384\|prf\|\|2113421C transposase [Burkholderia | |
| 1026 | 1346281 | CHITOOLIGOSACCHARIDOLYTIC BETA-N- | 0.7 |
| | | ACETYLGLUCOSAMINIDASE PRECURSOR (BETA- | |
| | | GLCNACASE) 3.2.1.-) - silkworm >gi\|998377\|bbs\|165703 (S77548) | |
| | | chitooligosaccharidolytic beta-N-acetylglucosaminidase, beta- | |
| 1027 | 3121722 | ALPHA-1D ADRENERGIC RECEPTOR (ALPHA 1D- | 0.18 |
| | | ADRENOCEPTOR) (ALPHA-1A ADRENERGIC RECEPTOR) | |
| | | receptor [mice, brain, Peptide, 562 aa] [Mus sp.] | |
| 1028 | 4071321 | (AF073954) Y-box protein MSY2 [Mus musculus] | 0.14 |
| 1029 | 1293835 | (U56965) C15H9.5 gene product [Caenorhabditis elegans] | 0.11 |
| 1030 | 2822157 | (AC004084) similar to GTPase-activating proteins; 35% similar to | 2e−013 |
| | | JC5047 (PID:g2136083) [Homo sapiens] | |
| 1040 | 1877301 | (Z92774) hypothetical protein Rv3570c | 9.7 |
| 1041 | 3024629 | GAMMA-SOLUBLE NSF ATTACHMENT PROTEIN (SNAP- | 7.8 |
| | | GAMMA) >gi\|423252\|pir\|\|S32369 gamma-SNAP protein - bovine | |
| | | >gi\|298669\|bbs\|127528 gamma soluble NSF attachment protein, | |
| | | gamma SNAP=N-ethyl-maleimide-sensitive fusion protein attachment | |
| 1042 | 731044 | UBIQUITIN CARBOXYL-TERMINAL HYDROLASE 7 | 7.6 |
| | | (UBIQUITIN THIOLESTERASE 7) probable membrane protein | |
| | | YIL156w - yeast (Saccharomyces cerevisiae) | |
| | | >gi\|557767\|emb\|CAA86122\|(Z38059) orf, len: 1071, CAI: 0.13, | |
| 1043 | 2190592 | (U78315) Hermansky-Pudlak syndrome protein homolog [Mus | 4.3 |
| 1044 | 2627435 | (AF013614) No definition line found [Fugu rubripes] | 3.5 |
| 1045 | 3881166 | (AL032641) Y5F2A.3 [Caenorhabditis elegans] | 2 |
| 1046 | 729918 | LA PROTEIN HOMOLOG (LA RIBONUCLEOPROTEIN) (LA | 0.009 |
| | | AUTOANTIGEN HOMOLOG) | |
| 1047 | 4106562 | (Z83819) dJ146H21.2 (similar to CYTOCHROME B-245 HEAVY | 3e−017 |
| | | CHAIN) [Homo sapiens] | |
| 1048 | 4557535 | down-regulated in adenoma protein down-regulated in adenoma | 4e−026 |
| | | (DRA) - human >gi\|291964 576-580, 579-583; acidic transcr. activ. | |
| | | domain 620-640,; homeobox motif 653-676 [Homo sapiens] | |
| 1049 | 4240235 | (AB020680) KIAA0873 protein [Homo sapiens] | 1e−037 |
| 1050 | 4240235 | (AB020680) KIAA0873 protein [Homo sapiens] | 2e−056 |
| 1065 | 586240 | VITAMIN K-DEPENDENT GAMMA-CARBOXYLASE (GAMMA- | 9.6 |
| | | GLUTAMYL CARBOXYLASE) 4.1.1.-) - bovine >gi\|289399 | |
| | | (L09726) gamma-carboxylase | |
| 1066 | 3413410 | (AL031231) 30S ribosomal protein S15 | 7.4 |
| 1067 | 1170758 | GALECTIN-3 (GALACTOSE-SPECIFIC LECTIN 3) (MAC-2 | 7.4 |
| | | ANTIGEN) (IGE-BINDING PROTEIN) (35 KD LECTIN) | |
| | | (CARBOHYDRATE BINDING PROTEIN 35) (CBP 35) | |
| 1068 | 867985 | (L39002) S1 gene product [Avian orthoreovirus] orthoreovirus] | 7.4 |
| 1069 | 127494 | METALLOTHIONEIN (MT) lucius] >gi\|62783\|emb\|CAA49636\| | 7.3 |
| | | (X70042) Metallothioein | |
| 1070 | 3150259 | (AL023634) palmitoyl-protein thioesterase precursor | 5.7 |
| | | [Schizosaccharomyces pombe] | |
| 1071 | 328647 | (M81729) tat [Human immunodeficiency virus type 1] | 4.3 |
| 1072 | 2605979 | (AF030027) 35 [Equine herpesvirus 4] | 3.3 |
| 1073 | 137532 | PROTEIN C2 >gi\|74386\|pir\|\|WZVZB6 59K HindIII-C protein - | 3.3 |
| | | vaccinia virus (strain WR) | |
| 1074 | 3769620 | (AF091563) olfactory receptor [Rattus norvegicus] | 3.3 |
| 1075 | 687367 | (U15829) envelope glycoprotein V3-V5 loop region [Human | 1.4 |
| | | immunodeficiency virus type 1] | |
| 1076 | 3915901 | TRANSCRIPTION INITIATION FACTOR TFIID 60 KD | 0.48 |
| | | SUBUNIT (TAFII-60) (TAFII60) | |
| 1077 | 2317934 | (U97553) unknown [murine herpesvirus 68] | 0.37 |
| 1080 | 3878966 | (Z68009) R09A8.1 [Caenorhabditis elegans] | 9.1 |
| 1081 | 2341037 | (AC000104) F19P19.17 [Arabidopsis thaliana] | 2.3 |
| 1084 | 4508019 | zinc finger protein 231 protein [Homo sapiens] | 8.9 |
| 1085 | 3877198 | (Z69903) predicted using Genefinder; Similarity to Rat casein kinase | 4 |
| | | I (SW:KC1D_RAT); cDNA EST EMBL:D65322 comes from this | |
| | | gene; cDNA EST EMBL:D68704 comes from this gene; cDNA EST | |
| | | yk475f2.5 comes from this gene [Caenorhabditis... | |
| 1086 | 171998 | (J02691) mitochondrial phenylalanyl-tRNA synthetase alpha subunit | 2.3 |
| | | precursor [Saccharomyces cerevisiae] | |
| 1087 | 135574 | LARGE TEGUMENT PROTEIN BPLF1 reading frame, 1 NXT/S, | 0.77 |
| | | analogous to VZV RF22 BPLF1 [Human herpesvirus 4] | |
| 1091 | 1718312 | (U75698) ORF K10 [Kaposi's sarcoma-associated herpesvirus] | 9.8 |
| 1092 | 1076012 | stress-sensitive restriction system protein 2 - Corynebacterium | 7.5 |
| | | glutamicum (ATCC 13032) >gi\|549844 restriction endonuclease | |
| | | which is stress-sensitive and ATP-dependent. It contains a typical | |
| 1093 | 882341 | (U24702) LRP1 [Arabidopsis thaliana] | 0.71 |
| 1094 | 3955011 | (AJ005438) beta adrenoreceptor B | 0.54 |
| 1095 | 1086900 | (U41278) contains similarity to G beta repeats | 1e−028 |
| 1096 | 4519908 | (AB018106) HrSmad1/5 [Halocynthia roretzi] | 6 |
| 1097 | 3043868 | (U95835) extracellular protein Exp5 precursor | 5.9 |
| 1098 | 2494294 | NEUROGENIC LOCUS NOTCH 3 PROTEIN | 3.5 |
| 1099 | 3879748 | (Z72514) predicted using Genefinder; similar to collagen; cDNA | 0.079 |
| | | EST EMBL:M89258 comes from this gene; cDNA EST | |
| | | EMBL:D68856 comes from this gene; cDNA EST yk232e11.3 comes | |
| | | from this gene; cDNA EST yk232e11.5 comes from this gene;... | |
| 1100 | 1123087 | (U42436) C49H3.3 gene product [Caenorhabditis elegans] | 5e−006 |
| 1101 | 3880809 | (AL021483) similar to Probable rabGAP domains; cDNA EST | 3e−011 |
| | | EMBL:D34945 comes from this gene; cDNA EST EMBL:D27313 | |
| | | comes from this gene; cDNA EST EMBL:D34829 comes from this | |
| | | gene; cDNA EST EMBL:D27312 comes from this gene; cDNA | |
| 1105 | 1772652 | (U70664) 2-keto-3-deoxygluconate kinase [Haloferax alicantei] | 5.8 |
| 1106 | 113000 | ACTIN BINDING PROTEIN | 5.5 |
| 1107 | 2702276 | (AC003033) putative beta-glucosidase [Arabidopsis thaliana] | 4.2 |
| 1108 | 1504006 | (D86966) similarto human ZFY protein. [Homo sapiens] | 4.2 |
| 1109 | 2337833 | (Z98604) hypothetical protein MLCB2052.27 | 1.8 |
| 1110 | 3122952 | TIPD PROTEIN >gi\|2407788 (AF019236) TipD [Dictyostelium | 6e−019 |
| 1112 | 4049344 | (AL034567) putative protein [Arabidopsis thaliana] | 6.8 |
| 1113 | 2143767 | glycoprotein - rat >gi\|986943 (L08134) glycoprotein [Rattus | 0.018 |
| | | norvegicus] norvegicus] | |
| 1116 | 200135 | (M63849) 2′- 5′oligoadenylate synthetase [Mus musculus] | 7.9 |
| 1119 | 133135 | MITOCHONDRIAL RIBOSOMAL PROTEIN VAR1 1-339) | 9.7 |
| 1120 | 1580768 | (X92688) NADH dehydrogenase [Pecten maximus] | 4.4 |
| 1121 | 4587868 | (AF069302) Orf2 [Pediococcus pentosaceus] | 1.5 |
| 1123 | 3851496 | (AF051933) cyclin T; positive elongation transcription factor b cyclin | 3.4 |
| | | subunit [Drosophila melanogaster] | |
| 1131 | 3877133 | (Z83109) predicted using Genefinder | 6 |
| 1132 | 118864 | DNA POLYMERASE (A PROTEIN) 2.7.7.7) - ground squirrel | 4.6 |
| | | hepatitis virus >gi\|325402 | |
| 1133 | 415638 | (U03388) cyclooxygenase 1 [Rattus rattus] | 4.5 |
| 1134 | 4100099 | (U93502) hypothetical protein 256 [Secale cereale] | 2.7 |
| 1135 | 2130154 | hypothetical protein 241 - maize chloroplast | 1.2 |
| 1136 | 4521280 | (AB011832) cytochrome c oxidase subunit I [Dicyema misakiense] | 0.94 |
| 1137 | 2706875 | (D85084) NCAM-180 [Cynops pyrrhogaster] | 0.68 |
| 1138 | 746516 | (U23517) D1022.7 [Caenorhabditis elegans] >gi\|3258651 elegans] | 0.061 |
| 1139 | 746516 | (U23517) D1022.7 [Caenorhabditis elegans] >gi\|3258651 elegans] | 0.061 |
| 1140 | 1938549 | (U97016) similar to drosophila Rlc1 gene product ribosomal protein | 5e−017 |
| | | L4 (YML4) (NID:g459259) | |
| 1141 | 1938549 | (U97016) similar to drosophila Rlc1 gene product ribosomal protein | 3e−017 |
| | | L4 (YML4) (NID:g459259) | |
| 1142 | 3851703 | (AF100421) p80 [Rattus norvegicus] | 1e−056 |
| 1143 | 4191810 | (AB006532) DNA helicase [Homo sapiens] | 8e−066 |
| 1144 | 3851703 | (AF100421) p80 [Rattus norvegicus] | 8e−070 |
| 1163 | 82620 | probable serine-type carboxypeptidase (EC 3.4.16.1) - wheat | 9.7 |
| 1164 | 3879471 | (Z69885) predicted using Genefinder | 9.6 |
| 1165 | 1075011 | mercury resistance regulatory protein KW20) | 9.5 |
| 1166 | 4490715 | (AL035680) putative protein [Arabidopsis thaliana] | 9.3 |
| 1167 | 487418 | (L20303) actin filament-associated protein [Gallus gallus] | 7.6 |
| 1168 | 560610 | trypsin inhibitor, WTI [Triticum aestivum=wheat, variety San | 7.5 |
| | | Pastore, endosperm, Peptide, 71 aa] | |
| 1169 | 3322954 | (AE001240) spermidine/putrescine ABC transporter, permease | 7.5 |
| | | protein (potB) [Treponema pallidum] | |
| 1170 | 1707179 | (U80839) ZC204.15 gene product [Caenorhabditis elegans] | 7.4 |
| 1171 | 209619 | (J01901) major coat protein A [Adeno associated virus 2] | 7.3 |
| 1172 | 1657233 | (D88529) serine acetyltransferase serine acetyltransferase [Spinacia | 7.3 |
| 1173 | 722375 | (U23139) similar to phospholipase ADRAB-B precursor | 7.2 |
| 1174 | 138018 | TAIL FIBER PROTEIN GP37 (RECEPTOR RECOGNIZING | 5.5 |
| | | PROTEIN) >gi\|76090\|pir\|\|TLBP74 tail fiber protein gp37 - phage T4 | |
| | | >gi\|15374\|emb\|CAA24228\|(V00863) gene 37 [coliphage T4] | |
| | | >gi\|215887 (J02509) tail fiber protein 37 [Bacteriophage T4] | |
| 1175 | 1001217 | (D64003) hypothetical protein | 4.4 |
| 1176 | 1175410 | HYPOTHETICAL 126.5 KD PROTEIN C31A2.16 IN | 4.4 |
| | | CHROMOSOME I >gi\|2130427\|pir\|\|S58108 hypothetical protein | |
| 1177 | 2829216 | (AF044287) delta adaptin subunit of AP-3 [Drosophila melanogaster] | 4.2 |
| 1178 | 1084374 | histone-lysine N-methyltransferase (EC 2.1.1.43) large chain N- | 4.1 |
| | | methyltransferase - garden pea carboxylase large subunit N- | |
| | | methyltransferase [Pisum sativum] | |
| 1179 | 567166 | (L03172) This CDS feature is included to show the translation of the | 3.8 |
| | | corresponding V_region. Presently translation qualifiers on V_region | |
| | | features are illegal. | |
| 1180 | 2492625 | HYPOTHETICAL 49.3 KD PROTEIN C09G5.1 IN | 3.2 |
| | | CHROMOSOME II >gi\|3874104\|emb\|CAA86760\| | |
| 1181 | 1065455 | (U40410) coded for by C. elegans cDNA yk9e10.5; coded for by C. | 2.6 |
| | | elegans cDNA yk9e10.3; multiple regions of similarity to EGF-like | |
| | | repeats and cysteine-rich repeats | |
| 1182 | 3406753 | (AF068182) B cell linker protein BLNK [Mus musculus] | 2.6 |
| 1183 | 1065455 | (U40410) coded for by C. elegans cDNA yk9e10.5; coded for by C. | 2.5 |
| | | elegans cDNA yk9e10.3; multiple regions of similarity to EGF-like | |
| | | repeats and cysteine-rich repeats | |
| 1184 | 4539761 | (AF118391) salivary peroxidase | 2.5 |
| 1185 | 101919 | mating-type locus protein b3 - smut fungus | 2.1 |
| 1186 | 329876 | (M32084) polyprotein [Hepatitis C virus] | 1.5 |
| 1187 | 3881059 | (AL023844) similar to Ion transport proteins [Caenorhabditis | 1.4 |
| 1188 | 2981221 | (AF053091) eyelid [Drosophila melanogaster] | 1.4 |
| 1189 | 4566750 | (AF115773) basic helix-loop-helix transcription factor Ndr1b [Danio | 1.4 |
| 1190 | 552072 | (M13235) abl-like putative oncogene; putative | 0.62 |
| 1191 | 1438951 | (U61842) cutinase negative acting protein [Fusarium solani f. sp. | 0.5 |
| 1192 | 3875904 | (Z70207) predicted using Genefinder; similar to collagen; cDNA | 0.025 |
| | | EST EMBL:D65905 comes from this gene; cDNA EST | |
| | | EMBL:D65858 comes from this gene; cDNA EST EMBL:D69306 | |
| | | comes from this gene; cDNA EST EMBL:D65755 comes from this | |
| 1193 | 3150503 | (AF067217) contains similarity to myosin head (motor) domains | 0.002 |
| | | (Pfam: myosin_head.hmm score: 602.72, 40.38 and 128.290) and | |
| | | phorbol esters/diacylglycerol binding domains (Pfam: DAG_PE- | |
| | | bind.hmm, score: 21.52 and 36.32) | |
| 1194 | 110849 | proline-rich protein - mouse proline-rich protein [Mus musculus] | 5e−005 |
| 1195 | 3435157 | (AF049910) TACC1 [Homo sapiens] | 7e−011 |
| 1196 | 3287336 | (AF014903) NADH dehydrogenase subunit 2 [Pan troglodytes] | 5e−011 |
| | | >gi\|3287338 (AF014904) NADH dehydrogenase subunit 2 [Pan | |
| 1197 | 4226073 | (AF125443) contains similarity to S. pombe phosphatidyl synthase | 2e−011 |
| | | (GB:Z28295) [Caenorhabditis elegans] | |
| 1198 | 868241 | (U29488) C56C10.3 gene product [Caenorhabditis elegans] | 2e−011 |
| 1199 | 3894323 | (AB020063) Keap1 [Mus musculus] | 5e−012 |
| 1200 | 466102 | PUTATIVE AMINOPEPTIDASE ZK353.6 IN CHROMOSOME III | 3e−014 |
| | | >gi\|1078908\|pir\|\|S44657 ZK353.6 protein - Caenorhabditis elegans | |
| | | >gi\|289760 (L15313) homology with leucine aminopeptidase; coded | |
| | | for by C. elegans cDNAs CE2F12 (GenBank: Z14714) and CE15D11 | |
| 1201 | 3608372 | (AF053768) brain specific cortactin-binding protein CBP90 [Rattus | 2e−016 |
| 1202 | 3293547 | (AF072709) putative oxidoreductase [Streptomyces lividans] | 2e−019 |
| 1203 | 4567068 | (AF125568) tumor suppressing STF cDNA 4 [Homo sapiens] | 3e−031 |
| 1204 | 4507851 | reserved >gi\|3928762\|dbj\|BAA34703\| | 1e−048 |
| 1205 | 3746791 | (AF081788) putative spliceosome associated protein [Homo sapiens] | 4e−051 |
| | | >gi\|3985930\|dbj\|BAA34863\|(AB020623) DAM1 [Homo sapiens] | |
| 1206 | 3851703 | (AF100421) p80 [Rattus norvegicus] | 2e−064 |
| 1207 | 4519883 | (AB017970) dipeptidyl peptidase III | 3e−071 |
| 1245 | 2370558 | (Z98763) putative Inositol polyphosphate phosphatase | 9.1 |
| | | [Schizosaccharomyces pombe] | |
| 1246 | 1076815 | NADH dehydrogenase subunit I - maize | 8.3 |
| 1247 | 409711 | (L25658) ORF [Blueberry scorch virus] | 8.3 |
| 1248 | 4323053 | (AF098293) pyruvate decarboxylase | 8.3 |
| 1249 | 2493794 | C4B-BINDING PROTEIN BETA CHAIN PRECURSOR | 8.2 |
| | | >gi\|2143627\|pir\|\|S57960 C4BP protein beta chain precursor - rat | |
| | | >gi\|899382\|emb\|CAA90392\|(Z50052) C4BP beta chain, leader | |
| 1250 | 1703342 | APOLIPOPROTEIN D PRECURSOR | 6.8 |
| 1251 | 133652 | PROBABLE RNA-DIRECTED RNA POLYMERASE polymerase | 6.6 |
| | | (EC 2.7.7.48) - southern bean mosaic virus mosaic virus] | |
| 1252 | 2128472 | hypothetical protein MJ0749 - Methanococcus jannaschii | 6.5 |
| | | >gi\|1591462 (U67520) conserved hypothetical protein | |
| 1253 | 2499265 | NADH-UBIQUINONE OXIDOREDUCTASE CHAIN 4 | 6.4 |
| | | >gi\|1182022\|emb\|CAA56534\|(X80245) ND4 [Locusta migratoria] | |
| 1254 | 1098322 | Ran/TC4-binding nucleopore protein [Homo sapiens] | 5.3 |
| 1255 | 283518 | homeotic protein smox-5 - fluke (Schistosoma mansoni) >gi\|161110 | 5.2 |
| | | (M85304) homeodomain protein | |
| 1256 | 119714 | EXTENSIN PRECURSOR (CELL WALL HYDROXYPROLINE- | 4.9 |
| | | RICH GLYCOPROTEIN) >gi\|82169\|pir\|\|S06733 hydroxyproline−rich | |
| | | glycoprotein precursor - common tobacco >gi\|19867\|emb\|CAA32090\| | |
| | | (X13885) extensin (AA 1-620) [Nicotiana tabacum] | |
| 1257 | 1176529 | HYPOTHETICAL 100.9 KD PROTEIN C34E10.3 IN | 4.8 |
| | | CHROMOSOME III >gi\|500726 (U10402) C34E10.3 gene product | |
| 1258 | 116922 | COPPER RESISTANCE PROTEIN B PRECURSOR | 3.8 |
| | | >gi\|77826\|pir\|\|B32018 copper resistance protein B precursor - | |
| 1259 | 1352916 | HYPOTHETICAL 19.0 KD PROTEIN IN NNF1-STE24 | 3.7 |
| | | INTERGENIC REGION >gi\|1077909\|pir\|\|S57138 hypothetical | |
| | | protein YJR115w - yeast (Saccharomyces cerevisiae) | |
| | | >gi\|1015833\|emb\|CAA89645\|(Z49615) ORF YJR115w | |
| 1260 | 184111 | (M20677) Kruppel-related protein (AA at 172) [Homo sapiens] | 3.7 |
| 1261 | 122137 | BOLA CLASS I HISTOCOMPATIBILITY ANTIGEN, ALPHA | 3.6 |
| | | CHAIN BL3-6 PRECURSOR antigen alpha chain precursor (BL3-6) | |
| 1262 | 1086822 | (U41263) this gene lies in the intron of T19D12.4 and on the opposite | 3 |
| | | strand; strong similarity to casein kinases and to C. elegans proteins | |
| | | CO3C10.2, F41G3.5 and ZK507.1 | |
| 1263 | 3257206 | (AP000003) 338aa long hypothetical protein | 3 |
| 1264 | 4493896 | (AL034558) predicted using hexExon; MAL3P2.18 (PFCO245c), | 2.8 |
| | | Hypothetical protein, len: 3934 aa | |
| 1265 | 732330 | GENERAL STRESS PROTEIN A Bacillus subtilis | 2.2 |
| | | >gi\|580866\|emb\|CAA51568\|(X73124) ipa-12d [Bacillus subtilis] | |
| 1266 | 4155225 | (AE001499) putative ROD SHAPE-DETERMINING PROTEIN | 2.1 |
| 1267 | 2224448 | (AB001684) ORF54c [Chlorella vulgaris] | 1.4 |
| 1268 | 3406654 | (AF079369) transcriptional repressor TUP1 [Dictyostelium | 1.4 |
| 1269 | 3335561 | (AF069736) PCAF associated factor 65 beta [Homo sapiens] | 1.3 |
| 1270 | 2326171 | (U62794) CDC42 GAP-related protein [Homo sapiens] | 1.3 |
| 1271 | 3406654 | (AF079369) transcriptional repressor TUP1 [Dictyostelium | 1.2 |
| 1272 | 2145956 | probable phosphomannomutase (EC 5.4.2.8) - Mycobacterium leprae | 1 |
| | | >gi\|467178 (U00022) u0308b; L308_F1_13 [Mycobacterium leprae] | |
| 1273 | 2649256 | (AE001012) GMP synthase (guaA-2) [Archaeoglobus fulgidus] | 1 |
| 1274 | 3451437 | (AL031350) putative secreted protein | 0.76 |
| 1275 | 1653006 | (D90910) hypothetical protein | 0.21 |
| 1276 | 1001821 | (D64005) nickel resistance [Synechocystis sp.] | 0.14 |
| 1277 | 1123087 | (U42436) C49H3.3 gene product [Caenorhabditis elegans] | 0.013 |
| 1278 | 1123087 | (U42436) C49H3.3 gene product [Caenorhabditis elegans] | 0.0004 |
| 1279 | 183894 | (M69054) insulin-like growth factor binding protein 6 | 7e−005 |
| 1280 | 3608372 | (AF053768) brain specific cortactin-binding protein CBP9O [Rattus | 9e−011 |
| 1281 | 3114818 | (AJ005891) JM2 [Homo sapiens] | 5e−011 |
| 1282 | 3608372 | (AF053768) brain specific cortactin-binding protein CBP90 [Rattus | 7e−016 |
| 1283 | 3608372 | (AF053768) brain specific cortactin-binding protein CBP9O [Rattus | 2e−016 |
| 1284 | 3649741 | (AJ000281) mucin [Homo sapiens] | 2e−031 |
| 1285 | 4240307 | (AB020716) KIAA0909 protein [Homo sapiens] | 1e−043 |
| 1286 | 4240307 | (AB020716) KIAA0909 protein [Homo sapiens] | 1e−043 |
| 1287 | 3649741 | (AJ000281) mucin [Homo sapiens] | 1e−047 |
| 1288 | 4557535 | down-regulated in adenoma protein down-regulated in adenoma | 1e−060 |
| | | (DRA) - human >gi\|291964 576-580, 579-583; acidic transcr. activ. | |
| | | domain 620-640,; homeobox motif 653-676 [Homo sapiens] | |
| 1324 | 3879589 | (Z50875) Proline rich domain; cDNA EST EMBL:D35637 comes | 9.9 |
| | | from this gene; cDNA EST yk322a3.5 comes from this gene; cDNA | |
| | | EST yk397b2.5 comes from this gene; cDNA EST yk348b11.5 | |
| | | comes from this gene; cDNA EST yk397b2.3 comes from... | |
| 1325 | 3879505 | (AL023816) T05G11.4 [Caenorhabditis elegans] | 9.4 |
| 1326 | 1346035 | FOS-RELATED ANTIGEN-1 >gi\|998348 | 8 |
| 1327 | 2648784 | (AE000981) dipeptide ABC transporter, dipeptide-binding protein | 7.9 |
| | | (dppA) [Archaeoglobus fulgidus] | |
| 1328 | 142774 | (L07023) delta-endotoxin [Bacillus thuringiensis] | 7.9 |
| 1329 | 2648784 | (AE000981) dipeptide ABC transporter, dipeptide-binding protein | 7.9 |
| | | (dppA) [Archaeoglobus fulgidus] | |
| 1330 | 3874201 | (Z81015) predicted using Genefinder | 7.7 |
| 1331 | 2358287 | (AF010404) ALR [Homo sapiens] | 7.7 |
| 1332 | 3881262 | (AL021175) Y6E2A.6 [Caenorhabditis elegans] | 6 |
| 1333 | 974143 | (L42542) RLIP76 protein [Homo sapiens] | 6 |
| 1334 | 3287188 | (Y10601) ankyrin-like protein [Homo sapiens] | 6 |
| 1335 | 730684 | UBIQUITIN-PROTEIN LIGASE RSP5 yeast (Saccharomyces | 4.6 |
| | | cerevisiae)>gi\|603364 (U18916) Rsp5p [Saccharomyces cerevisiae] | |
| 1336 | 1255865 | (U53340) coded for by C. elegans cDNA yk39e8.5; weakly similar to | 3.6 |
| | | C. elegans proteins F09G8.4 and F02E8.6 | |
| 1337 | 2687999 | (AE001123) B. burgdorferi predicted coding region BB0110 | 3.3 |
| 1338 | 623044 | (L38896) This CDS feature is included to show the translation of the | 3.1 |
| | | corresponding V_region. Presently translation qualifiers on V_region | |
| | | features are illegal | |
| 1339 | 987050 | (X65335) lacZ [Cloning vector pSV-beta-Galactosidase Control] | 2.7 |
| 1340 | 1136390 | (D79986) similar to human DNA-binding protein 5. [Homo sapiens] | 2.6 |
| 1341 | 3599395 | (AF083072) histone H1 DNA binding protein [Cenarchaeum | 1.4 |
| 1342 | 1144514 | (U34781) Antho-LWamidII preprohormone [Anthopleura | 1.2 |
| | | elegantissima] >gi\|1586846\|prf\|\|2204411A prepro-hormone | |
| 1343 | 3406654 | (AF079369) transcriptional repressor TUP1 [Dictyostelium | 0.98 |
| 1344 | 3132825 | (AF063403) putative cytosine-5 DNA methyltransferase [Zea mays] | 0.89 |
| 1345 | 1754989 | (U47661) proline-rich protein PRP2 precursor [Lupinus luteus] | 0.078 |
| 1346 | 728834 | !!!! ALU SUBFAMILY SB2 WARNING ENTRY | 0.025 |
| 1347 | 1123087 | (U42436) C49H3.3 gene product [Caenorhabditis elegans] | 0.021 |
| 1348 | 3608372 | (AF053768) brain specific cortactin-binding protein CBP90 [Rattus | 1e−009 |
| 1349 | 4507029 | sodium bicarbonate cotransporter 2 >gi\|3097316\|dbj\|BAA25898\| | 5e−016 |
| | | (AB012130) sodium bicarbonate cotransporter2 [Homo sapiens] | |
| 1350 | 2781381 | (AC004013) Similar to rabbit A-kinase-anchoring protein sapiens] | 1e−029 |
| 1351 | 4584423 | (AJ131693) AKAP450 protein [Homo sapiens] | 3e−052 |
| 1359 | 1518135 | (U66260) multidrug resistance related protein 1 | 5.2 |
| 1362 | 3450974 | (AF082496) interleukin-2 [Marmota monax] | 6.8 |
| 1363 | 1123087 | (U42436) C49H3.3 gene product [Caenorhabditis elegans] | 0.001 |
| 1364 | 119760 | COAGULATION FACTOR X PRECURSOR 3.4.21.6) precursor - | 2 |
| | | chicken >gi\|222870\|dbj\|BAA00724\| | |
| 1367 | 3861111 | (AJ235272) unknown [Rickettsia prowazekii] | 5.5 |
| 1370 | 117143 | CYTOCHROME P450 1A1 (CYPIA1) rainbow trout >gi\|213780 | 7.1 |
| | | (M21310) cytochrome P450IA1 | |
| 1371 | 3915910 | HYPOTHETICAL 36.3 KD PROTEIN IN NRDC-MOBD | 5.4 |
| | | INTERGENIC REGION >gi\|1667570 (U76612) unknown | |
| 1372 | 1708230 | HOMEOBOX PROTEIN ABDOMINAL-A | 4.1 |
| 1373 | 3024292 | RHODOPSIN >gi\|2290717 (AF000947) rhodopsin [Sepia officinalis] | 5.3 |
| 1374 | 2996206 | (AF053723) region 2 capsular polysaccharide biosynthesis protein | 2.3 |
| | | [Actinobacillus pleuropneumoniae] | |
| 1375 | 2228750 | (U93868) RNA polymerase III subunit [Homo sapiens] | 5e−011 |
| 1379 | 79960 | hypothetical 30.5 K protein - Enterococcus faecalis plasmid pAM-beta | 2e−025 |
| | | 1 >gi\|3023044 (AF007787) orfC | |
| 1380 | 465532 | HYPOTHETICAL 14.2 KD PROTEIN IN BLAB 3′REGION | 5.7 |
| | | >gi\|282541\|pir\|\|C41855 orf3 - Streptomyces cacaoi | |
| | | >gi\|217001\|dbj\|BAA00776\|(D00937) regulatory protein for beta- | |
| 1381 | 3913279 | COAT PROTEIN PRECURSOR (CAPSID PROTEIN) >gi\|535774 | 0.66 |
| | | (L09205) capsid protein [Tobacco ringspot virus] | |
| 1389 | 4493762 | (AL034368) predicted using hexExon; L779.1, Serine/threonine | 4.6 |
| | | protein kinase, len: 1359 aa; Similarity to protein kinases. A.thaliana | |
| | | NPK1-related protein kinase (TR:O22041) BLAST score: 303, sum | |
| 1390 | 131002 | PROLINE-RICH PROTEIN MP-3 >gi\|200549 | 0.1 |
| 1391 | 632098 | isp3 protein - fission yeast | 4.5 |
| 1392 | 2496862 | HYPOTHETICAL 6.1 KD PROTEIN C03B1.10 IN | 6e−006 |
| | | CHROMOSOME X >gi\|1072242 (U40952) C03B1.10 gene product | |
| 1393 | 3851703 | (AF100421) p80 [Rattus norvegicus] | 4e−056 |
| 1399 | 4493762 | (AL034368) predicted using hexExon; L779.1, Serine/threonine | 4.3 |
| | | protein kinase, len: 1359 aa; Similarity to protein kinases. A.thaliana | |
| | | NPK1-related protein kinase (TR:O22041) BLAST score: 303, sum | |
| 1400 | 129648 | PAIRED BOX PROTEIN PAX-1 | 4.3 |
| 1401 | 4115789 | (D89861) cytochrome C-type biogenesis protein CCMF | 1.9 |
| | | [Cyanidioschyzon merolae] | |
| 1402 | 135514 | T-CELL RECEPTOR BETA CHAIN PRECURSOR precursor (ANA | 0.034 |
| 1403 | 1086833 | (U41264) coded for by C. elegans cDNA CEESN26F; coded for by | 3e−009 |
| | | C. elegans cDNA CEESI89F; similar to 60S acidic ribosomal protein | |
| | | Po (L10) [Caenorhabditis elegans] | |
| 1404 | 4104168 | (AF033339) UNC-45 [Caenorhabditis briggsae] | 7e−013 |
| 1410 | 1175805 | HYPOTHETICAL PROTEIN HI1452 Haemophilus influenzae | 7.3 |
| | | (strain Rd KW20) >gi\|1574290 | |
| 1411 | 1708230 | HOMEOBOX PROTEIN ABDOMINAL-A | 4.2 |
| 1412 | 127774 | MYOSIN II HEAVY CHAIN, NON MUSCLE heavy chain | 0.56 |
| | | [Dictyostelium discoideum] | |
| 1413 | 728833 | !!!! ALU SUBFAMILY SB1 WARNING ENTRY | 0.48 |
| 1414 | 3080645 | (AC004611) Hsp27 ERE-TATA-binding protein [Homo sapiens] | 3e−008 |
| 1415 | 3687476 | (AL031786) putative atp dependent rna helicase | 1e−014 |
| | | [Schizosaccharomyces pombe] | |
| 1416 | 4557535 | down-regulated in adenoma protein down-regulated in adenoma | 5e−060 |
| | | (DRA) - human >gi\|291964 576-580, 579-583; acidic transcr. activ. | |
| | | domain 620-640,; homeobox motif 653-676 [Homo sapiens] | |
| 1421 | 2088648 | (AF002109) hypothetical protein [Arabidopsis thaliana] | 6.9 |
| 1422 | 1707768 | (Y08256) orf c01038 [Sulfolobus solfataricus] | 5.3 |
| 1423 | 3370996 | (AB008674) mBlm [Mus musculus] | 4 |
| 1424 | 3878128 | (Z69663) Weak similarity to Cotton isocitrate lyase | 1.8 |
| | | (SW:ACEA_GOSHI); cDNA EST EMBL:D33259 comes from this | |
| | | gene; cDNA EST EMBL:D35346 comes from this gene; cDNA EST | |
| | | EMBL:D36032 comes from this gene; cDNA EST EMBL:D73253 | |
| 1425 | 83996 | hypothetical protein 1 (CYb-COII intergenic region) - | 1.8 |
| | | Sauroleishmania tarentolae mitochondrion uncertain [Leishmania | |
| 1426 | 1322210 | (U26347) immunogobulin kappa, VJ region [Homo sapiens] | 1.4 |
| 1427 | 3043810 | (U88273) NADH dehydrogenase subunit 4 [Sceloporus aeneus] | 0.46 |
| 1428 | 3080645 | (AC004611) Hsp27 ERE-TATA-binding protein [Homo sapiens] | 0.0007 |
| 1429 | 4506223 | proteasome (prosome, macropain) 26S subunit, non-ATPase, 13 | 1e−066 |
| | | >gi\|36183431\|dbj\|BAA33214\| | |
| 1438 | 2944430 | (AF050157) butyrophilin-like [Mus musculus] | 8.9 |
| 1439 | 1652823 | (D90908) hypothetical protein | 3 |
| 1440 | 119714 | EXTENSIN PRECURSOR (CELL WALL HYDROXYPROLINE- | 0.9 |
| | | RICH GLYCOPROTEIN) >gi\|82169\|pir\|\|S06733 hydroxyproline-rich | |
| | | glycoprotein precursor - common tobacco >gi\|19867\|emb\|CAA32090\| | |
| | | (X13885) extensin (AA 1-620) [Nicotiana tabacum] | |
| 1441 | 509813 | (L29010) ORFB [Cryphonectria hypovirus] | 0.78 |
| 1442 | 238617 | (S64572) nonfibrillar collagen Partial, 907 aa] [Strongylocentrotus | 0.2 |
| 1443 | 3522970 | (U42390) Trio [Homo sapiens] | 0.12 |
| 1449 | 3328726 | (AE001303) ATP Synthase Subunit E [Chlamydia trachomatis] | 8.6 |
| 1450 | 501174 | (L33965) MHC class II protein [Morone saxatilis] | 8.5 |
| 1451 | 2661685 | (AL009199) hypothetical protein SC7B7.10 | 8.5 |
| 1452 | 1575684 | (U70316) IonA [Dictyostelium discoideum] | 8.4 |
| 1453 | 2128472 | hypothetical protein MJ0749 - Methanococcus jannaschii | 5 |
| | | >gi\|1591462 (U67520) conserved hypothetical protein | |
| 1454 | 1549329 | (U52845) class IV chitinase EP3-1/H5 [Daucus carota] | 4.9 |
| 1455 | 3845167 | (AE001390) hypothetical protein [Plasmodium falciparum] | 2.9 |
| 1456 | 2833306 | PUTATIVE IMPORTIN ALPHA SUBUNIT for by C. elegans | 1.7 |
| | | cDNA yk117h8.5; coded for by C. elegans cDNA yk8f10.5; coded | |
| | | for by C. elegans cDNA yk134a4.5; coded for by C. elegans cDNA | |
| | | yk53c4.5; coded for by C. elegans cDNA yk8f10.3; coded for by C. | |
| 1463 | 1731097 | HYPOTHETICAL 34.2 KD PROTEIN F07F6.2 IN | 8.3 |
| | | CHROMOSOME III >gi\|746449 (U23486) No definition line found | |
| 1464 | 4321805 | (AF063232) variant 1 major surface glycoprotein [Pneumocystis | 8.3 |
| 1465 | 4557059 | (AC007154) hypothetical protein | 4.8 |
| 1466 | 139372 | ENDOPROTEASE (LATE L3 23 KD PROTEIN) adenovirus type 7] | 4.8 |
| 1467 | 8509 | (X04813) CAD protein (AA 1 -2236) [Drosophila melanogaster] | 3.6 |
| 1468 | 422690 | myosin-binding protein H - chicken | 2e−005 |
| 1472 | 265108 | (S54379) sucrose-phosphate synthase, SPS oleracea] | 2.1 |
| 1473 | 2842704 | HYPOTHETICAL 73.3 KD PROTEIN C6G9.14 IN | 1.6 |
| | | CHROMOSOME I >gi\|1644326\|emb\|CAB03616.1\|protein | |
| 1474 | 113109 | NEURONAL ACETYLCHOLINE RECEPTOR PROTEIN, NON- | 0.7 |
| | | ALPHA-3 CHAIN PRECURSOR (GFN-ALPHA-3) non-alpha-3 | |
| | | chain precursor - goldfish >gi\|212956 auratus] | |
| 1475 | 4557535 | down-regulated in adenoma protein down-regulated in adenoma | 1e−051 |
| | | (DRA) - human >gi\|291964 576-580, 579-583; acidic transcr. activ. | |
| | | domain 620-640,; homeobox motif 653-676 [Homo sapiens] | |
| 1485 | 2315339 | (AF016438) contains similarity to C4-type zinc fingers | 9.9 |
| 1486 | 549664 | HYPOTHETICAL 23.0 KD PROTEIN IN IXR1-TFA1 | 4.4 |
| | | INTERGENIC REGION >gi\|539152\|pir\|\|S37847 hypothetical protein | |
| | | YKL030w - yeast (Saccharomyces cerevisiae) | |
| | | >gi\|486029\|emb\|CAA81864\|(Z28029) ORF YKLO3Ow | |
| 1487 | 4204234 | (AF035379) MADS-box protein 2 [Lolium temulentum] | 0.51 |
| 1488 | 126390 | LORICRIN >gi\|110649\|pir\|\|A35628 loricrin - mouse >gi\|198871 | 0.14 |
| | | (M34398) loricrin [Mus musculus] >gi\|520480 (U09189) loricrin | |
| 1489 | 4557535 | down-regulated in adenoma protein down-regulated in adenoma | 4e−050 |
| | | (DRA) - human >gi\|291964 576-580, 579-583; acidic transcr. activ. | |
| | | domain 620-64O,; homeobox motif 653-676 [Homo sapiens] | |
| 1492 | 1707768 | (Y08256) orf c01038 [Sulfolobus solfataricus] | 4.3 |
| 1493 | 4376203 | (U35226) putative cytochrome P-450 | 4.3 |
| 1494 | 3721884 | (AB016248) sterol-C5-desaturase [Mus musculus] | 4.2 |
| 1495 | 131000 | ACIDIC PROLINE-RICH PROTEIN PRECURSOR salivary protein | 3.3 |
| | | [Rattus norvegicus] | |
| 1496 | 3123638 | (AJ223069) TCF-3 protein [Mus musculus] | 0.49 |
| 1497 | 854065 | (X83413) U88 [Human herpesvirus 6] | 5e−010 |
| 1501 | 126679 | GALECTIN-3 (GALACTOSE-SPECIFIC LECTIN 3) (MAC-2 | 0.21 |
| | | ANTIGEN) (IGE-BINDING PROTEIN) (35 KD LECTIN) | |
| | | (CARBOHYDRATE BINDING PROTEIN 35) (CBP 35) | |
| | | GALACTOSIDE-BINDING LECTIN) >gi\|1072481\|pir\|\|A28651 | |
| 1502 | 4106562 | (Z83819) dJ146H21.2 (similar to CYTOCHROME B-245 HEAVY | 1e−011 |
| | | CHAIN) [Homo sapiens] | |
| 1504 | 265108 | (S54379) sucrose-phosphate synthase, SPS oleracea] | 1.6 |
| 1505 | 1177322 | (X95466) CPG2 protein [Rattus norvegicus] | 0.0002 |
| 1507 | 3878057 | (Z99942) similar to von Willebrand factor type A domain; cDNA | 0.039 |
| | | EST yk412d4.5 comes from this gene; cDNA EST yk412d4.3 comes | |
| | | from this gene [Caenorhabditis elegans] | |
| 1509 | 2394509 | (AF024503) No definition line found [Caenorhabditis elegans] | 6.1 |
| 1510 | 2827872 | (U79229) NADH dehydrogenase subunit F [Tradescantia zebrina] | 4.6 |
| 1511 | 3004653 | (AF017777) tweety [Drosophila melanogaster] | 3.7 |
| 1512 | 3873773 | (Z83316) Similarity to S.pombe hypothetical protein C2F7.02C | 1.6 |
| 1513 | 586295 | HYPOTHETICAL 33.8 KD PROTEIN IN YSW1-RIB7 | 0.081 |
| | | INTERGENIC REGION >gi\|626526\|pir\|\|S46023 hypothetical protein | |
| | | YBR152w - yeast (Saccharomyces cerevisiae) | |
| | | >gi\|536480\|emb\|CAA85111\|(Z36021) ORF YBR152w | |
| 1519 | 3882147 | (AB018256) KIAA0713 protein [Homo sapiens] | 9.9 |
| 1520 | 2117181 | (Z95584) mcr [Mycobacterium tuberculosis] | 7.3 |
| 1521 | 1169862 | G-BOX BINDING FACTOR 3 >gi\|600863 thaliana] | 6 |
| 1522 | 3257950 | (AP000006) 236aa long hypothetical protein | 6 |
| 1523 | 4138677 | (AJ009814) polymerase [Viral hemorrhagic septicemia virus] | 5.9 |
| 1524 | 2736413 | (AF039044) No definition line found [Caenorhabditis elegans] | 5.7 |
| 1525 | 2058691 | (U94836) ERPROT 213-21 [Homo sapiens] | 2.7 |
| 1526 | 347124 | (L20967) phosphodiesterase [Homo sapiens] | 2 |
| 1527 | 3913674 | FERRIC REDUCTASE TRANSMEMBRANE COMPONENT 3 | 2 |
| | | PRECURSOR >gi\|2132959\|pir\|\|S67293 probable membrane protein | |
| | | YOR381w - yeast (Saccharomyces cerevisiae) | |
| | | >gi\|1420821\|emb\|CAA99713\|(Z75289) ORF YOR381w | |
| 1528 | 1086636 | (U41013) similar to phosphoribosylaminoimidazolecarboxamide | 0.9 |
| | | formyltransferase [Caenorhabditis elegans] | |
| 1529 | 2495706 | HYPOTHETICAL PROTEIN KIAA0136 product is novel. [Homo | 0.86 |
| 1530 | 4007775 | (AL034486) hypothetical fungal binuclear cluster domain protein | 0.65 |
| | | [Schizosaccharomyces pombe] | |
| 1531 | 113671 | !!!! ALU CLASS F WARNING ENTRY !!!! | 0.39 |
| 1532 | 4098124 | (U73522) AMSH [Homo sapiens] | 8e−008 |
| 1533 | 1174546 | SYNAPTOTAGMIN II | 5e−010 |
| 1534 | 4139228 | (AF109134) 7-60 [Homo sapiens] >gi\|4139272 (AF112980) 7-60 | 5e−044 |
| 1535 | 2564328 | (AB006628) KIAA0290 [Homo sapiens] | 3e−050 |
| 1536 | 3668141 | (AJ007398) PBK1 protein [Homo sapiens] | 3e−057 |
| 1544 | 3256484 | (AP000001) 158aa long hypothetical protein [Pyrococcus horikoshii] | 9.1 |
| 1545 | 2735945 | (AF015193) NADH dehydrogenase subunit 3 [Onchocerca volvulus] | 7.5 |
| 1546 | 3983015 | (AF096056) antigen receptor [Ginglymostoma cirratum] | 3.2 |
| 1547 | 1552169 | (D42138) PIG-B [Homo sapiens] | 3 |
| 1548 | 2440180 | (Z99531) ubiquitin system protein | 2.4 |
| 1549 | 3882159 | (AB018262) KIAA0719 protein [Homo sapiens] | 1.8 |
| 1550 | 3004653 | (AF017777) tweety [Drosophila melanogaster] | 1.3 |
| 1551 | 77657 | hypothetical 30.1 K protein - Pseudomonas aeruginosa | 0.26 |
| 1552 | 4098124 | (U73522) AMSH [Homo sapiens] | 0.002 |
| 1553 | 3979818 | (Z49967) cDNA EST EMBL:T00743 comes from this gene; cDNA | 0.001 |
| | | EST EMBL:D69356 comes from this gene; cDNA EST | |
| | | EMBL:D65790 comes from this gene; cDNA EST EMBL:D70463 | |
| | | comes from this gene; cDNA EST EMBL:D66620 comes from this | |
| 1554 | 3550295 | (AJ009947) putative ATPase [Homo sapiens] | 1e−008 |
| 1555 | 4507507 | UNKNOWN >gi\|3929583 (AF098162) timeless homolog [Homo | 1e−035 |
| 1562 | 3152567 | (AC002986) Contains similarity to membrane associated salt- | 8.4 |
| | | inducible protein gb\|AF007269 from A. thaliana. | |
| 1563 | 4493978 | (AL034559) predicted using hexExon; MAL3P7.21 (PFC0960c), | 5 |
| | | Hypothetical protein, len: 1929 aa | |
| 1564 | 1169862 | G-BOX BINDING FACTOR 3 >gi\|600863 thaliana] | 4.7 |
| 1565 | 140550 | HYPOTHETICAL 259 KD PROTEIN (ORF 2136) | 3.7 |
| | | >gi\|81341\|pir\|\|A05037 hypothetical protein 2136 - liverwort | |
| | | (Marchantia polymorpha) chloroplast polymorpha] | |
| 1566 | 2224354 | (AB001684) ORF104 [Chlorella vulgaris] | 0.76 |
| 1567 | 106322 | hypothetical protein (L1H 3′ region) - human | 0.53 |
| 1568 | 3522937 | (AC004411) unknown protein [Arabidopsis thaliana] | 0.4 |
| 1569 | 4097433 | (U61084) phorbolin 3 [Homo sapiens] | 4e−017 |
| 1578 | 1944122 | (AB002531) SSU1p [Saccharomyces cerevisiae] | 9.7 |
| 1579 | 4127662 | (Y11176) fructosidase [Cichorium intybus] | 5.3 |
| 1580 | 119515 | EPENDYMIN I PRECURSOR (EPD-I) rainbow trout >gi\|213412 | 4.4 |
| | | (M93697) ependymin | |
| 1581 | 1439625 | (U64598) weakly similar to S. cervisiae PTM1 precursor | 2.6 |
| 1582 | 2183261 | (AF002133) MAV264 [Mycobacterium avium] | 2.3 |
| 1583 | 2388564 | (AC000098) ESTs gb\|AA042402, gb\|ATTS1380 come from this gene. | 1.9 |
| | | [Arabidopsis thaliana] | |
| 1584 | 1381091 | (U51723) V-SERA 1 [Plasmodium vivax] | 9.8 |
| 1587 | 2496317 | HYPOTHETICAL PROTEIN MG144 HOMOLOG Mycoplasma | 0.65 |
| | | pneumoniae (SGC3) (ATCC 29342) >gi\|1674380 genitalium | |
| 1588 | 2496317 | HYPOTHETICAL PROTEIN MG144 HOMOLOG Mycoplasma | 0.64 |
| | | pneumoniae (SGC3) (ATCC 29342) >gi\|1674380 genitalium | |
| 1589 | 3184073 | (AL023779) hypothetical protein | 8e−009 |
| 1590 | 3560165 | (AL031525) hypothetical protein | 3.7 |
| 1612 | 133594 | RNA-DIRECTED RNA POLYMERASE (ORF1B) 2.7.7.48) - avian | 9.7 |
| | | infectious bronchitis virus (strain Beaudette) >gi\|292953 (M95169) | |
| | | pol protein [Avian infectious bronchitis virus] >gi\|331173 (M94356) | |
| | | ORF 1b encodes a polypeptide of potential mol. wt. 300,000. | |
| 1613 | 3043622 | (AB011121) KIAA0549 protein [Homo sapiens] | 6.8 |
| 1614 | 72870 | translation elongation factor eEF-1 alpha chain factor 1-alpha (AA 1- | 5.6 |
| | | 461) [Mus musculus] | |
| 1615 | 2315365 | (AF016441) No definition line found [Caenorhabditis elegans] | 5.3 |
| 1616 | 586120 | TRICHOHYALIN >gi\|539701\|pir\|\|A45973 trichohyalin - human | 4.9 |
| | | >gi\|292836 (L09190) trichohyalin | |
| 1617 | 281206 | nuclear antigen EBNA-3B - human herpesvirus 4 | 3.9 |
| 1618 | 2146731 | FK506-binding protein - Arabidopsis thaliana | 3.5 |
| 1619 | 3876045 | (Z81506) similar to Lectin C-type domain short and long forms, CUB | 3.5 |
| | | domain [Caenorhabditis elegans] | |
| 1620 | 2833191 | LACCASE 4 PRECURSOR precursor - Rhizoctonia solani | 3.3 |
| | | >gi\|1150568\|emb\|CAA91042\| | |
| 1621 | 2981221 | (AF053091) eyelid [Drosophila melanogaster] | 3.2 |
| 1622 | 1118179 | (U42304) chitin synthase [Phytophthora capsici] | 2.9 |
| 1623 | 1351576 | HYPOTHETICAL PROTEIN MG419 Mycoplasma genitalium | 2.9 |
| 1624 | 320309 | AL2 protein - beet curly top virus >gi\|210683 | 2.5 |
| 1625 | 1523997 | (X99510) CDK4/6 kinase [Drosophila melanogaster] | 2.1 |
| 1626 | 3646450 | (AL031603) conserved hypothetical protein. | 2 |
| 1627 | 3599342 | (AF081112) ORF2 [Mus musculus domesticus] | 1.9 |
| 1628 | 2114473 | (U96963) p140mDia [Mus musculus] | 1.5 |
| 1629 | 3638957 | (AC004877) sco-spondin-mucin-like; similar to P98167 uncertain | 1 |
| | | [Homo sapiens] | |
| 1630 | 2558942 | (AF024714) interferon-inducible protein [Homo sapiens] | 0.93 |
| 1631 | 3257709 | (AP000005) 115aa long hypothetical protein | 0.69 |
| 1632 | 346430 | adenylylcyclase type V-alpha - dog >gi\|290082 | 0.46 |
| 1633 | 4263788 | (AC006068) hypothetical protein | 0.3 |
| 1634 | 2134199 | myosin I beta - bullfrog >gi\|602138 (U14549) myosin I beta [Rana | 0.044 |
| | | catesbeiana] >gi\|603692 (U14382) myosin I beta [Rana catesbeiana] | |
| 1635 | 2134199 | myosin I beta - bullfrog >gi\|602138 (U14549) myosin I beta [Rana | 0.036 |
| | | catesbeiana] >gi\|603692 (U14382) myosin I beta [Rana catesbeiana] | |
| 1636 | 3776027 | (AJ010475) RNA helicase [Arabidopsis thaliana] | 2e−006 |
| 1637 | 134787 | ATP-DEPENDENT RRNA HELICASE SPB4 cerevisiae] | 1e−015 |
| | | >gi\|836754\|dbj\|BAA09238\|(D50617) rRNA helicase | |
| | | [Saccharomyces cerevisiae] SPB4 [Saccharomyces cerevisiae] | |
| 1638 | 134787 | ATP-DEPENDENT RRNA HELICASE SPB4 cerevisiae] | 4e−016 |
| | | >gi\|836754\|dbj\|BAA09238\|(D50617) rRNA helicase | |
| | | [Saccharomyces cerevisiae] SPB4 [Saccharomyces cerevisiae] | |
| 1639 | 4454698 | (AF070661) HSPC005 [Homo sapiens] | 2e−016 |
| 1640 | 4185892 | (AL033125) 1-evidence=predicted by content; 1- | 6e−021 |
| | | method=genefinder; 084; 1-evidence_end; 2-evidence=predicted by | |
| | | match; 2-match_accession=SWISS-PROT:P38205; 2- | |
| | | match_description=HYPOTHETICAL 77.9 KD PROTEIN IN | |
| 1641 | 3043548 | (AB011084) KIAA0512 protein [Homo sapiens] | 9e−041 |
| 1643 | 2435574 | (AF026209) similar to C. elegans olfactory receptor ODR-10 | 7.3 |
| | | (NID:g1235900) [Caenorhabditis elegans] | |
| 1644 | 3880252 | (Z82055) similar to Zinc finger, C4 type | 5.7 |
| 1645 | 3023744 | PUTATIVE FLAGELLA-RELATED PROTEIN C | 4.5 |
| 1646 | 267478 | HYPOTHETICAL 64.3 KD PROTEIN IN RPS3 3′ REGION | 2 |
| | | (ORF516) >gi\|419731\|pir\|\|S34525 hypothetical protein 516 (rps3 3′ | |
| | | region) - Euglena gracilis chloroplast >gi\|2673852\|emb\|CAA50104\| | |
| | | (X70810) orf516; ttg start [Euglena gracilis] | |
| 1647 | 82551 | hypothetical 237 protein (psbA 5′ region) - rye chloroplast (fragment) | 0.49 |
| 1648 | 1255792 | (U51993) similar drosophila lethal (2) giant larvae protien | 0.09 |
| | | (SP:Q08470) [Caenorhabditis elegans] | |
| 1663 | 627837 | All-1 protein +GTE form - mouse (fragment) | 9.6 |
| 1664 | 4115789 | (D89861) cytochrome C-type biogenesis protein CCMF | 6.3 |
| | | [Cyanidioschyzon merolae] | |
| 1665 | 942618 | (U31081) MnxB [Bacillus sp.] | 6 |
| 1666 | 220579 | (D00570) open reading frame (196 AA) [Mus musculus] | 5.8 |
| 1667 | 2120082 | hypothetical protein II (retroposon LINE-1) | 4.9 |
| 1668 | 2120082 | hypothetical protein II (retroposon LINE-1) | 4.8 |
| 1669 | 862343 | (L10908) Gcap1 gene product [Mus musculus] marker protein [Mus | 4.6 |
| 1670 | 1168540 | ARGININOSUCCINATE SYNTHASE argininosuccinate synthetase | 4.5 |
| | | (argG) homolog - Haemophilus influenzae (strain Rd KW20) | |
| 1671 | 160385 | (M63277) malaria antigen [Plasmodium falciparum] | 4 |
| 1672 | 1177607 | (X92485) pva1 [Plasmodium vivax] | 3.8 |
| 1673 | 2497678 | ZYXIN >gi\|1430883\|emb\|CAA67510\| | 2.5 |
| 1674 | 4049518 | (AL031852) conserved hypothetical protein | 2.1 |
| 1675 | 135514 | T-CELL RECEPTOR BETA CHAIN PRECURSOR precursor (ANA | 0.55 |
| 1676 | 729942 | LIPASE 1 PRECURSOR (TRIACYLGLYCEROL LIPASE) | 0.45 |
| | | >gi\|538817\|pir\|\|A47081 triacylglycerol lipase | |
| 1677 | 4503629 | coagulation factor XII (Hageman factor) | 0.27 |
| | | >gi\|119763\|sp\|P00748\|FA12_HUMAN COAGULATION FACTOR | |
| | | XII PRECURSOR (HAGEMAN FACTOR) (HAF) 3.4.21.38) | |
| | | precursor - human >gi\|180357 (M17466) coagulation factor XII | |
| 1678 | 1708392 | CASPASE-5 PRECURSOR (ICH-3 PROTEASE) (TY PROTEASE) | 4e−006 |
| | | (ICE(REL)-III) enzyme ICErel-III - human >gi\|903936 (U28015) | |
| | | cysteine protease [Homo sapiens] | |
| 1679 | 3550295 | (AJ009947) putative ATPase [Homo sapiens] | 1e−008 |
| 1680 | 3875400 | (Z73906) cDNA EST EMBL:M88866 comes from this gene | 1e−009 |
| | | [Caenorhabditis elegans] | |
| 1681 | 3874685 | (Z78539) Similarity to S.pombe hypothetical protein C4G8.04 | 6e−010 |
| | | (SW:YAD4_SCHPO); cDNA EST EMBL:D27846 comes from this | |
| | | gene; cDNA EST EMBL:D27845 comes from this gene; cDNA EST | |
| | | yk202h7.3 comes from this gene; cDNA EST yk202h7.5 come... | |
| 1682 | 3979818 | (Z49967) cDNA EST EMBL:T00743 comes from this gene; cDNA | 2e−015 |
| | | EST EMBL:D69356 comes from this gene; cDNA EST | |
| | | EMBL:D65790 comes from this gene; cDNA EST EMBL:D70463 | |
| | | comes from this gene; cDNA EST EMBL:D66620 comes from this | |
| 1683 | 4584877 | (AF068302) choline/ethanolaminephosphotransferase [Homo | 5e−020 |
| 1685 | 3914503 | ASPARTATE CARBAMOYLTRANSFERASE 2 PRECURSOR | 9.9 |
| | | (ASPARTATE TRANSCARBAMYLASE 2) (ATCASE 2) sativum] | |
| 1686 | 1845893 | (U69426) envelope glycoprotein [Human immunodeficiency virus | 9.7 |
| 1687 | 1730629 | HYPOTHETICAL 43.0 KD PROTEIN B0361.6 IN | 5e−014 |
| | | CHROMOSOME III >gi\|458954 (U00031) similar to H. marismortui | |
| | | hypothetical 23.1 kd protein in HMAL3 5′region [Caenorhabditis | |
| 1690 | 2133580 | gag polyprotein - red flour beetle retrotransposon Woot >gi\|805076 | 1.1 |
| | | (U09586) ORF 1 | |
| 1695 | 126296 | LINE-1 REVERSE TRANSCRIPTASE HOMOLOG protein | 6 |
| | | [Nycticebus coucang] | |
| 1696 | 111816 | probable pol polyprotein-related protein 4 - rat | 5.3 |
| | | >gi\|56590\|emb\|CAA37647\|(X53581) ORF4 [Rattus norvegicus] | |
| 1697 | 1517923 | (U51117) ascospore maturation 1 protein [Neurospora crassa] | 5.3 |
| 1698 | 2924552 | (AL022018) 1-evidence=predicted by content; 1- | 6e−012 |
| | | method=genefinder; 084; 1-method_score=165.48; 1-evidence_end; 2- | |
| | | evidence=predicted by match; 2-match_accession=AA264666; 2- | |
| | | match_description=LD08351.5prime LD Drosophila melanogaste... | |
| 1699 | 1663706 | (D87685) similar to human transcription factor TFIIS (S34159). | 2e−014 |
| 1700 | 3879850 | (Z81592) predicted using Genefinder | 3e−034 |
| 1707 | 3184085 | (AL023781) cytochrome c oxidase polypeptide v precursor | 8.7 |
| | | [Schizosaccharomyces pombe] | |
| 1708 | 3845206 | (AE001400) novel protein kinase [Plasmodium falciparum] | 3.9 |
| 1709 | 283435 | hypothetical protein DGF-1 - Trypanosoma cruzi cruzi] | 3 |
| 1710 | 3329623 | (AF078790) No definition line found [Caenorhabditis elegans] | 1.3 |
| 1711 | 2459999 | (AF013108) tubulin Uni3 [Chlamydomonas reinhardtii] | 0.32 |
| 1712 | 1705447 | BACTENECIN 7 PRECURSOR (BAC7) aries] | 0.26 |
| 1713 | 3043596 | (AB011108) KIAA0536 protein [Homo sapiens] | 4e−022 |
| 1714 | 3879850 | (Z81592) predicted using Genefinder | 3e−033 |
| 1724 | 987050 | (X65335) lacZ [Cloning vector pSV-beta-Galactosidase Control] | 7.6 |
| 1725 | 1334582 | (X55026) ORF16; no ATG start codon | 6.8 |
| 1726 | 2981631 | (AB012223) ORF2 [Canis familiaris] | 5.2 |
| 1727 | 628300 | gene F protein - rinderpest virus virus] | 4.8 |
| 1728 | 4008417 | (Z77669) Similarity to Human aminopeptidase N | 3.9 |
| | | (SW:AMPN_HUMAN); cDNA EST EMBL:D36412 comes from this | |
| | | gene; cDNA EST EMBL:D37688 comes from this gene; cDNA EST | |
| | | EMBL:D34550 comes from this gene; cDNA EST EMBL:D33568 | |
| 1729 | 1170083 | PROBABLE GLOBAL TRANSACTIVATOR transactivator-like | 3.4 |
| | | protein [Autographa californica nucleopolyhedrovirus] | |
| 1730 | 3757727 | (AL022727) dJ80I19.7 (olfactory receptor-like protein (hs6M1-3)) | 3.3 |
| | | [Homo sapiens] | |
| 1731 | 126296 | LINE-1 REVERSE TRANSCRIPTASE HOMOLOG protein | 1.2 |
| | | [Nycticebus coucang] | |
| 1732 | 130702 | LARGE STRUCTURAL PHOSPHOPROTEIN | 0.97 |
| | | PHOSPHOPROTEIN) (BPP) >gi\|73955\|pir\|\|XPBEA9 large structural | |
| | | phosphoprotein pp 150 - human cytomegalovirus phosphoprotein 150 | |
| 1733 | 1706571 | EPITHELIAL CHLORIDE CHANNEL PROTEIN calcium-activated | 0.87 |
| | | chloride channel [Bos taurus] | |
| 1734 | 2981631 | (AB012223) ORF2 [Canis familiaris] | 0.56 |
| 1735 | 1280072 | (U55366) coded for by C. elegans cDNA yk85a2.5; coded for by C. | 0.44 |
| | | elegans cDNA yk85a2.3; Similar to cuticlin | |
| 1736 | 2496862 | HYPOTHETICAL 6.1 KD PROTEIN C03B1.10 IN | 0.001 |
| | | CHROMOSOME X >gi\|1072242 (U40952) C03B1.10 gene product | |
| 1737 | 2228750 | (U93868) RNA polymerase III subunit [Homo sapiens] | 1e−007 |
| 1738 | 4503511 | UNKNOWN >gi\|3264861 (U97670) eukaryotic translation initiation | 4e−031 |
| | | factor eIF3, p35 subunit [Homo sapiens] | |
| 1739 | 4503511 | UNKNOWN >gi\|3264861 (U97670) eukaryotic translation initiation | 1e−032 |
| | | factor eIF3, p35 subunit [Homo sapiens] | |
| 1740 | 113667 | !!!! ALU CLASS B WARNING ENTRY !!!! | 5.3 |
| 1742 | 2736375 | (AF039040) No definition line found [Caenorhabditis elegans] | 6 |
| 1743 | 2736375 | (AF039040) No definition line found [Caenorhabditis elegans] | 3.4 |
| 1744 | 1002672 | (U30261) G protein beta subunit-like; Method: conceptual translation | 5e−024 |
| | | supplied by author [Schistosoma mansoni] | |
| 1752 | 4200151 | (AJ011538) hypothetical protein virus] | 3.9 |
| 1753 | 134528 | SLP1 PROTEIN (VACUOLAR PROTEIN SORTING PROTEIN 33) | 1.6 |
| | | >gi\|101624\|pir\|\|A34708 SLP1 protein SLP1 protein [Saccharomyces | |
| | | cerevisiae] >gi\|173185 | |
| 1754 | 1175386 | HYPOTHETICAL 37.7 KD PROTEIN C18B11.06 IN | 0.13 |
| | | CHROMOSOME I >gi\|2130289\|pir\|\|S58305 hypothetical protein | |
| | | SPAC18B11.06 - fission yeast pombe] | |
| 1756 | 2909528 | (AL021932) PPE [Mycobacterium tuberculosis] | 4.9 |
| 1760 | 3876797 | (Z81531) cDNA EST EMBL:D66579 comes from this gene; cDNA | 3.9 |
| | | EST EMBL:D70408 comes from this gene; cDNA EST yk263d3.5 | |
| | | comes from this gene; cDNA EST yk275c1.5 comes from this gene; | |
| | | cDNA EST EMBL:C10270 comes from this gene [Caenorh... | |
| 1770 | 3608418 | (AF086634) cyclin B [Dreissena polymorpha] | 8.9 |
| 1771 | 416868 | CYTOKINE RECEPTOR COMMON BETA CHAIN PRECURSOR | 4.3 |
| | | >gi\|110595\|pir\|\|A35782 cytokine receptor common beta chain | |
| | | precursor - mouse >gi\|309101 (M34397) interleukin 3 receptor-like | |
| 1772 | 126296 | LINE-1 REVERSE TRANSCRIPTASE HOMOLOG protein | 2.9 |
| | | [Nycticebus coucang] | |
| 1773 | 728834 | !!!! ALU SUBFAMILY SB2 WARNING ENTRY | 1.9 |
| 1774 | 728836 | !!!! ALU SUBFAMILY SP WARNING ENTRY | 0.3 |
| 1775 | 126296 | LINE-1 REVERSE TRANSCRIPTASE HOMOLOG protein | 0.001 |
| | | [Nycticebus coucang] | |
| 1776 | 3879850 | (Z81592) predicted using Genefinder | 2e−037 |
| 1777 | 4155993 | (AE001560) putative [Helicobacter pylori J99] | 1.4 |
| 1782 | 225047 | reverse transcriptase related protein [Homo sapiens] | 2.2 |
| 1790 | 3785977 | (AC005560) putative growth regulator protein | 9.4 |
| 1791 | 3257681 | (AP000005) 617aa long hypothetical prolyl endopeptidase | 7.8 |
| | | [Pyrococcus horikoshii] | |
| 1792 | 2317972 | (U97553) capsid protein [murine herpesvirus 68] | 1.5 |
| 1793 | 1085738 | cytochrome-c oxidase (EC 1.9.3.1) chain I - Trypanoplasma borreli | 0.014 |
| | | mitochondrion (SGC6) >gi\|563140 borreli] | |
| 1794 | 4493973 | (AL034559) predicted using hexExon; MAL3P7.14 (PFC0925w), | 0.002 |
| | | Hypothetical protein, len: 489 aa | |
| 1800 | 461796 | CYTOCHROME C OXIDASE POLYPEPTIDE III honeybee | 9.1 |
| | | mitochondrion (SGC4) >gi\|552443 (L06178) cytochrome oxidase | |
| | | subunit 3 [Apis mellifera ligustica] | |
| 1801 | 1850592 | (U88295) carnitine palmitoyltransferase II [Rattus norvegicus] | 9 |
| 1802 | 207854 | (M35837) alpha-amylase/alpha-galactosidase fusion protein | 8.7 |
| 1803 | 2136989 | Ksp-cadherin - rabbit>gi\|902886 (U28945) Ksp-cadherin | 3.4 |
| | | [Oryctolagus cuniculus] cuniculus] | |
| 1804 | 288448 | (X06487) bcl2-Ig fusion gene [Homo sapiens] | 3.4 |
| 1805 | 1657752 | (U62325) FE65-like protein [Homo sapiens] | 2.1 |
| 1806 | 2981631 | (AB012223) ORF2 [Canis familiaris] | 5e−007 |
| 1807 | 4502103 | annexin 31 XXXI) [Homo sapiens] | 1e−043 |
| 1808 | 2149830 | (U92864) maturase [Quercus rubra] | 9.6 |
| 1809 | 2981631 | (AB012223) ORF2 [Canis familiaris] | 0.046 |
| 1810 | 728832 | !!!! ALU SUBFAMILY SB WARNING ENTRY | 0.035 |
| 1813 | 3649769 | (Z98547) predicted using hexExon; MAL3P3.17 (PFC0420w), | 2.1 |
| | | Calcium-dependent protein kinase, len: 591 aa; Similarity to calcium- | |
| | | dependent protein kinases. P.falciparum calcium-dependent protein | |
| | | kinase CDPK2 (TR:O15865) BLAST Sco... | |
| 1814 | 126296 | LINE-1 REVERSE TRANSCRIPTASE HOMOLOG protein | 1.6 |
| | | [Nycticebus coucang] | |
| 1815 | 197968 | (M26361) LINE/Ig H-chain fusion protein [Mus musculus] | 0.036 |
| 1816 | 2496862 | HYPOTHETICAL 6.1 KD PROTEIN C03B1.10 IN | 0.001 |
| | | CHROMOSOME X >gi\|1072242 (U40952) C03B1.10 gene product | |
| 1817 | 135514 | T-CELL RECEPTOR BETA CHAIN PRECURSOR precursor (ANA | 0.0006 |
| 1818 | 3043596 | (AB011108) KIAA0536 protein [Homo sapiens] | 4e−016 |
| 1821 | 728838 | !!!! ALU SUBFAMILY SX WARNING ENTRY | 7 |
| 1822 | 1177607 | (X92485) pva1 [Plasmodium vivax] | 0.23 |
| 1823 | 106323 | hypothetical protein (L1H 5′ region) - human | 0.071 |
| 1824 | 2981631 | (AB012223) ORF2 [Canis familiaris] | 2e−009 |
| 1825 | 1086860 | (U41272) Similar to man(9)-alpha-mannosidase. | 1e−032 |
| 1829 | 2258350 | (AF007261) Sec Y-type transporter protein [Reclinomonas | 2.9 |
| 1830 | 1169643 | FMRFAMIDE-RELATED NEUROPEPTIDES PRECURSOR | 0.026 |
| | | >gi\|416208 (U03137) neuropeptide precursor FMRFamide-related | |
| 1834 | 585424 | LIPOATE-PROTEIN LIGASE A 6.3.4.-) A - Escherichia coli | 7.1 |
| | | >gi\|504496 (L27665) lipoate-protein ligase A [Escherichia coli] | |
| 1835 | 2981631 | (AB012223) ORF2 [Canis familiaris] | 0.044 |
| 1841 | 4584540 | (AL049608) putative protein [Arabidopsis thaliana] | 7.3 |
| 1842 | 4493975 | (AL034559) predicted using hexExon; MAL3P7.17 (PFC0940c), | 3 |
| | | Hypothetical protein, len: 807 aa | |
| 1843 | 343539 | (M14820) NADH dehydrogenase subunit 4 [Trypanosoma brucei] | 1.7 |
| 1844 | 3335138 | (AF047441) RNA polymerase I 40 kD subunit [Homo sapiens] | 1 |
| 1852 | 2612897 | (AF015825) hypothetical ABC transporter [Bacillus subtilis] | 5.9 |
| | | >gi\|2633579\|emb\|CAB13082\|(Z99110) similar to amino acid ABC | |
| | | transporter (ATP-binding protein) | |
| 1853 | 728836 | !!!! ALU SUBFAMILY SP WARNING ENTRY | 1.2 |
| 1854 | 113668 | !!!! ALU CLASS C WARNING ENTRY !!!! | 0.17 |
| 1855 | 728836 | !!!! ALU SUBFAMILY SP WARNING ENTRY | 0.05 |
| 1856 | 106323 | hypothetical protein (L1H 5′ region) - human | 0.048 |
| 1857 | 2981631 | (AB012223) ORF2 [Canis familiaris] | 0.0002 |
| 1859 | 729785 | HEXOSE TRANSPORTER HXT8 YJL214w - yeast (Saccharomyces | 0.12 |
| | | cerevisiae) cerevisiae] >gi\|1015600\|emb\|CAA89511\|(Z49489) ORF | |
| | | YJL214w [Saccharomyces cerevisiae] | |
| 1861 | 1791243 | (U83119) ORF2 consensus sequence encoding endonuclease and | 4e−006 |
| | | reverse transcriptase minus RNaseH [Rattus norvegicus] | |
| 1862 | 854065 | (X83413) U88 [Human herpesvirus 6] | 2e−006 |
| 1863 | 1707274 | (U80931) strong similarity to class-III of pyridoxal-phoshate- | 3e−032 |
| | | dependent aminotransferases | |
| 1871 | 4049828 | (AF063866) ORF MSV145 hypothetical protein [Melanoplus | 5.6 |
| | | sanguinipes entomopoxvirus] | |
| 1872 | 1109853 | (U41538) weak similarity to the S. cerevisiae activator 1 05 KD | 4.5 |
| | | subunit (replication factor C 95 KD subunit) | |
| 1873 | 113668 | !!!! ALU CLASS C WARNING ENTRY !!!! | 3.6 |
| 1874 | 1684995 | (U20663) NADH dehydrogenase subunit [Encyclia tampensis] | 1.6 |
| 1875 | 3599325 | (AF081106) ORF1 [Mus musculus domesticus] | 0.36 |
| 1876 | 160379 | (M63274) malaria antigen [Plasmodium falciparum] | 0.16 |
| 1877 | 728837 | !!!! ALU SUBFAMILY SQ WARNING ENTRY | 0.12 |
| 1878 | 2134199 | myosin I beta - bullfrog >gi\|602138 (U14549) myosin I beta [Rana | 0.036 |
| | | catesbeiana] >gi\|603692 (U14382) myosin I beta [Rana catesbeiana] | |
| 1879 | 1872200 | (U22376) alternatively spliced product using exon 13A | 0.009 |
| 1880 | 1938524 | (U97012) contains similarity to a ground domain, also weakly similar | 0.17 |
| | | to drosophila fork head domain transcription factor SLP1 | |
| | | (SP:P32030) [Caenorhabditis elegans] | |
| 1882 | 3510507 | (AF032382) metalloprotease-disintegrin [Xenopus laevis] | 0.77 |
| 1883 | 728837 | !!!! ALU SUBFAMILY SQ WARNING ENTRY | 0.33 |
| 1884 | 854065 | (X83413) U88 [Human herpesvirus 6] | 1e−007 |
| 1886 | 728833 | !!!! ALU SUBFAMILY SB1 WARNING ENTRY | 7.4 |
| 1887 | 1353178 | PUTATIVE SERINE/THREONINE PROTEIN PHOSPHATASE | 7.2 |
| | | C27B7.6 IN CHROMOSOME IV serine/threonine protein | |
| | | phosphatase [Caenorhabditis elegans] | |
| 1888 | 728837 | !!!! ALU SUBFAMILY SQ WARNING ENTRY | 7e−009 |
| 1890 | 435942 | (U04295) DNA-binding factor of bZIP class [Oryza sativa] | 1 |
| 1891 | 728832 | !!!! ALU SUBFAMILY SB WARNING ENTRY | 0.94 |
| 1892 | 3599347 | (AF081114) ORF2 [Mus musculus domesticus] | 0.55 |
| 1893 | 728837 | !!!! ALU SUBFAMILY SQ WARNING ENTRY | 0.056 |
| 1894 | 1196432 | (M22333) unknown protein [Homo sapiens] | 0.002 |
| 1895 | 728838 | !!!! ALU SUBFAMILY SX WARNING ENTRY | 0.004 |
| 1901 | 728831 | !!!! ALU SUBFAMILY J WARNING ENTRY | 8 |
| 1902 | 3646450 | (AL031603) conserved hypothetical protein. | 7e−028 |
| 1903 | 2213560 | (Z97052) hypothetical protein | 5e−026 |
| 1905 | 3002527 | (AF010144) neuronal thread protein AD7c-NTP [Homo sapiens] | 0.066 |
| 1906 | 2072977 | (U93574) putative p150 [Homo sapiens] | 0.022 |
| 1907 | 728835 | !!!ALU SUBFAMILY SC WARNING ENTRY | 0.019 |
| 1908 | 4153886 | (AB013357) 49 kDa zinc finger protein [Mus musculus] | 2e−005 |
| 1910 | 2072974 | (U93573) p40 [Homo sapiens] | 2 |
| 1911 | 728837 | !!!! ALU SUBFAMILY SQ WARNING ENTRY | 0.011 |
| 1914 | 4572297 | (AF071799) T-cell surface glycoprotein CD4 precursor | 6 |
| 1915 | 1350904 | RIBOSOME RECYCLING FACTOR (RIBOSOME RELEASING | 3.7 |
| | | FACTOR) (RRF) >gi\|1361841\|pir\|\|A64248 ribosome releasing factor | |
| | | Mycoplasma genitalium (SGC3) releasing factor (frr) [Mycoplasma | |
| 1916 | 728831 | !!!! ALU SUBFAMILY J WARNING ENTRY | 1.4 |
| 1917 | 728832 | !!!! ALU SUBFAMILY SB WARNING ENTRY | 0.0006 |
| 1918 | 126296 | LINE-1 REVERSE TRANSCRIPTASE HOMOLOG protein | 4e−005 |
| | | [Nycticebus coucang] | |
| 1919 | 1791243 | (U83119) ORF2 consensus sequence encoding endonuclease and | 4e−009 |
| | | reverse transcriptase minus RNaseH [Rattus norvegicus] | |
| 1920 | 126295 | LINE-1 REVERSE TRANSCRIPTASE HOMOLOG | 0.006 |
| 1921 | 348317 | ATPase 6 - Sauroleishmania tarentolae | 7.7 |
| 1923 | 1723547 | HYPOTHETICAL 61.8 KD PROTEIN C12B10.03 IN | 8.6 |
| | | CHROMOSOME I>gi\|1262416\|emb\|CAA94693\| | |
| 1924 | 1177607 | (X92485) pva1 [Plasmodium vivax] | 4.9 |
| 1925 | 3068583 | (AF000580) Rep-like [Dictyostelium discoideum] | 2.4 |
| 1926 | 2120082 | hypothetical protein II (retroposon LINE-1) | 0.21 |
| 1927 | 1335198 | (X03145) pot. ORF III [Homo sapiens] | 0.0003 |
| 1930 | 2134328 | ECH - chicken >gi\|1037160\|dbj \|BAA08364\| | 8.3 |
| 1931 | 4502377 | B-cell growth factor 1 (12 kD) | 0.094 |
| 1932 | 3417289 | (U95740) Unknown gene product [Homo sapiens] | 0.065 |
| 1933 | 565080 | (U14550) SThM [Homo sapiens] | 0.006 |
| 1934 | 728831 | !!!! ALU SUBFAMILY J WARNING ENTRY | 7e−006 |
| 1935 | 3649741 | (AJ000281) mucin [Homo sapiens] | 5e−020 |
| 1936 | 3551821 | (AF058803) mucin 4 [Homo sapiens] | 8e−021 |
| 1937 | 4519443 | (AB017430) kinesin-like DNA binding protein [Homo sapiens] | 3e−029 |
| 1939 | 1710216 | (U79260) unknown [Homo sapiens] | 1e−005 |
| 1940 | 728837 | !!!! ALU SUBFAMILY SQ WARNING ENTRY | 5e−008 |
| 1942 | 4494012 | (AL034559) predicted using hexExon; MAL3P7.32 (PFC1010w), | 4.5 |
| | | Hypothetical protein, len: 1322 aa | |
| 1943 | 220578 | (D00570) open reading frame (251 AA) [Mus musculus] | 0.077 |
| 1944 | 2731377 | (U28739) similar to alcohol dehydrogenase/ribitol dehydrogenase | 1e−028 |
| | | [Caenorhabditis elegans] | |
| 1945 | 4587207 | (AB020527) Na/PO4 cotransporter homolog | 4e−034 |
| 1948 | 728832 | !!!! ALU SUBFAMILY SB WARNING ENTRY | 0.21 |
| 1949 | 929913 | (V01442) ribosomal protein S8 [Xenopus laevis] | 0.066 |
| 1950 | 728831 | !!!! ALU SUBFAMILY J WARNING ENTRY | 2e−006 |
| 1951 | 1125754 | (U42833) coded for by C. elegans cDNA cm16f6; coded for by C. | 1e−028 |
| | | elegans cDNA CEESU63F; similar to S. cerevisiae SOF1 protein | |
| | | (SP:P33750) [Caenorhabditis elegans] | |
| 1952 | 4587207 | (AB020527) Na/PO4 cotransporter homolog | 6e−047 |
| 1953 | 4587207 | (AB020527) Na/PO4 cotransporter homolog | 2e−051 |
| 1955 | 2981631 | (AB012223) ORF2 [Canis familiaris] | 0.027 |
| 1956 | 4138064 | (Y18301) reverse transcriptase [Ovine pulmonary adenocarcinoma | 0.0002 |
| 1961 | 4507991 | zinc finger protein 140 (clone pHZ-39) | 8.6 |
| | | >gi\|1731416\|sp\|P52738\|Z140_HUMAN ZINC FINGER PROTEIN | |
| | | 140 >gi\|2136409\|pir\|\|C57785 zinc finger protein ZNF140 - human | |
| | | >gi\|487787 (U09368) zinc finger protein ZNF140 [Homo sapiens] | |
| 1962 | 1711541 | SERINE/THREONINE PROTEIN KINASE SSK2 (SUPPRESSOR | 5.9 |
| | | OF SENSOR KINASE 2) SSK2 gene product [Saccharomyces | |
| 1963 | 3236252 | (AC004684) CER1-like protein [Arabidopsis thaliana] | 3 |
| 1964 | 728836 | !!!! ALU SUBFAMILY SP WARNING ENTRY | 0.001 |
| 1965 | 4138070 | (Y18303) reverse trascriptase [Ovine pulmonary adenocarcinoma | 0.0002 |
| 1966 | 114128 | ADP-RIBOSYLATION FACTOR 6 1-175) [Gallus gallus] | 9e−011 |
| 1967 | 2293566 | (AF012896) ADP-ribosylation factor 1 [Oryza sativa] | 2e−012 |
| 1969 | 728831 | !!!! ALU SUBFAMILY J WARNING ENTRY | 0.091 |
| 1970 | 59977 | (Z14310) tripartite fusion transcript PLA2L | 2e−005 |
| 1971 | 126296 | LINE-1 REVERSE TRANSCRIPTASE HOMOLOG protein | 9 |
| | | [Nycticebus coucang] | |
| 1972 | 4155181 | (AE001495) putative TYPE II RESTRICTION ENZYME | 4.8 |
| 1973 | 2239204 | (Z97209) vacuolar carboxypeptidase | 0.008 |
| 1974 | 3342107 | (AF075269) nef protein [Simian immunodeficiency virus] | 6.9 |
| 1978 | 423149 | X-linked retinopathy protein (C-terminal, clone XEH.8c) - human | 0.66 |
| | | (fragment) >gi\|299471\|bbs\|129340 XEH.8c} [human, Peptide Partial, | |
| 1979 | 1791243 | (U83119) ORF2 consensus sequence encoding endonuclease and | 0.025 |
| | | reverse transcriptase minus RNaseH [Rattus norvegicus] | |
| 1980 | 126296 | LINE-1 REVERSE TRANSCRIPTASE HOMOLOG protein | 0.011 |
| | | [Nycticebus coucang] | |
| 1981 | 728831 | !!!! ALU SUBFAMILY J WARNING ENTRY | 0.0009 |
| 1982 | 1791243 | (U83119) ORF2 consensus sequence encoding endonuclease and | 4e−006 |
| | | reverse transcriptase minus RNaseH [Rattus norvegicus] | |
| 1984 | 1903379 | (U52073) differentially repressed by testosterone and | 3.2 |
| | | dihydrotestosterone [Mus musculus] | |
| 1985 | 1903379 | (U52073) differentially repressed by testosterone and | 3.1 |
| | | dihydrotestosterone [Mus musculus] | |
| 1986 | 3599339 | (AF081111) ORF2 [Mus musculus domesticus] | 2e−007 |
| 1990 | 728835 | !!!! ALU SUBFAMILY SC WARNING ENTRY | 5.9 |
| 1991 | 729093 | CCAAT DISPLACEMENT PROTEIN (HOMEOBOX PROTEIN | 0.018 |
| | | CLOX) (CLOX-1) >gi\|423173\|pir\|\|S33121 homeotic protein Clox - | |
| | | dog (fragment) >gi\|2202\|emb\|CAA48782\| | |
| 1992 | 126295 | LINE-1 REVERSE TRANSCRIPTASE HOMOLOG | 0.004 |
| 1993 | 1125754 | (U42833) coded for by C. elegans cDNA cm16f6; coded for by C. | 1e−030 |
| | | elegans cDNA CEESU63F; similar to S. cerevisiae SOF1 protein | |
| | | (SP:P33750) [Caenorhabditis elegans] | |
| 1994 | 3789771 | (AF055386) MHC class I related protein precursor | 1.9 |
| 1995 | 728832 | !!!! ALU SUBFAMILY SB WARNING ENTRY | 3.1 |
| 1996 | 3023729 | FRUCTOSE-1,6-BISPHOSPHATASE fructose-1,6-bisphosphatase | 0.21 |
| | | [Mus musculus] | |
| 1997 | 3182916 | ADP-RIBOSYLATION FACTOR factor [Plasmodium falciparum] | 2e−015 |
| | | >gi\|1932731 (U57370) ADP-ribosylation factor [Plasmodium | |
| 2000 | 113668 | !!!! ALU CLASS C WARNING ENTRY !!!! | 1 |
| 2001 | 728836 | !!!! ALU SUBFAMILY SP WARNING ENTRY | 0.0003 |
| 2002 | 59977 | (Z14310) tripartite fusion transcript PLA2L | 7e−007 |
| 2003 | 91035 | laminin receptor homolog - mouse 295) [Mus musculus] | 0.12 |
| | | >gi\|228997\|prf\|\|1815216A laminin receptor [Cricetinae gen. sp.] | |
| 2005 | 4506545 | regulator of mitotic spindle assembly 1 | 3.7 |
| | | >gi\|1350799\|sp\|P49646\|RMS1_HUMAN REGULATOR OF | |
| 2006 | 1619936 | (U70935) reverse transcriptase [Peromyscus maniculatus] | 0.015 |
| 2007 | 728837 | !!!! ALU SUBFAMILY SQ WARNING ENTRY | 0.007 |
| 2008 | 728831 | !!!! ALU SUBFAMILY J WARNING ENTRY | 0.0002 |
| 2009 | 728831 | !!!! ALU SUBFAMILY J WARNING ENTRY | 5e−006 |
| 2010 | 3157938 | (AC002131) Similar to seryl-tRNA synthetase gb\|U10400 from S | 5e−029 |
| | | cerevisiae. EST gb\|N96627 comes from this gene. | |
| 2011 | 2496862 | HYPOTHETICAL 6.1 KD PROTEIN C03B1.10 IN | 0.17 |
| | | CHROMOSOME X >gi\|1072242 (U40952) C03B1.10 gene product | |
| 2012 | 2443342 | (D88764) alpha 2 type I collagen [Rana catesbeiana] | 1.2 |
| 2013 | 1546035 | (U64570) myelin/oligodendrocyte glycoprotein-16.3 kD(a) precursor | 0.15 |
| | | [Homo sapiens] >gi\|1546037 (U64571) myelin/oligodendrocyte | |
| | | glycoprotein-16.3 kD(b) precursor | |
| 2014 | 728831 | !!!! ALU SUBFAMlLY J WARNING ENTRY | 0.006 |
| 2015 | 113668 | !!!! ALU CLASS C WARNING ENTRY !!!! | 0.003 |
| 2016 | 728838 | !!!! ALU SUBFAMILY SX WARNING ENTRY | 0.0003 |
| 2017 | 728837 | !!!! ALU SUBFAMILY SQ WARNING ENTRY | 1e−005 |
| 2019 | 3002527 | (AF010144) neuronal thread protein AD7c-NTP [Homo sapiens] | 1e−005 |
| 2021 | 728838 | !!!! ALU SUBFAMILY SX WARNING ENTRY | 3e−005 |
| 2022 | 2879805 | (AL021813) hypothetical protein | 0.6 |
| 2023 | 1711659 | TCP1-CHAPERONIN COFACTOR A taurus] | 0.003 |
| 2024 | 113667 | !!!! ALU CLASS B WARNING ENTRY !!!! | 0.0007 |
| 2025 | 113667 | !!!! ALU CLASS B WARNING ENTRY !!!! | 0.0007 |
| 2026 | 87765 | hypothetical L1 protein (third intron of gene TS) - human | 0.0003 |
| | | >gi\|364964\|prf\|\|1510254A L1 repetitive element ORF [Homo | |
| 2027 | 4009460 | (AF039401) calcium-dependent chloride channel-1 [Homo sapiens] | 2e−011 |
| 2028 | 1730840 | PUTATIVE CYSTEINYL-TRNA SYNTHETASE C29E6.06C | 2e−035 |
| | | (CYSTEINE-TRNA LIGASE) (CYSRS) YNL247w - yeast | |
| 2030 | 4200165 | (Y16262) neutral invertase [Daucus carota] | 5.7 |
| 2031 | 1150834 | (U42471) Wiscott-Aldrich Syndrome protein homolog [Mus | 0.032 |
| 2032 | 113669 | !!!! ALU CLASS D WARNING ENTRY !!!! | 0.002 |
| 2033 | 728831 | !!!! ALU SUBFAMILY J WARNING ENTRY | 0.0008 |
| 2034 | 1657758 | (U66707) densin-180 [Rattus norvegicus] | 1e−024 |
| 2035 | 4165313 | (AB022083) SOX30 protein [Homo sapiens] | 0.42 |
| 2036 | 1657758 | (U66707) densin-180 [Rattus norvegicus] | 1e−024 |
| 2037 | 3193336 | (AF069301) DBI-related protein [Homo sapiens] | 7e−055 |
| 2038 | 126296 | LINE-1 REVERSE TRANSCRIPTASE HOMOLOG protein | 8e−008 |
| | | [Nycticebus coucang] | |
| 2039 | 2981631 | (AB012223) ORF2 [Canis familiaris] | 6e−008 |
| 2040 | 4098124 | (U73522) AMSH [Homo sapiens] | 3e−018 |
| 2042 | 113668 | !!!! ALU CLASS C WARNING ENTRY !!!! | 9e−006 |
| 2043 | 728837 | !!!! ALU SUBFAMILY SQ WARNING ENTRY | 5e−009 |
| 2044 | 3123312 | ZINC FINGER PROTEIN 142 (KIAA0236) Human zinc finger | 0.7 |
| | | protein (ZNF142) [Homo sapiens] | |
| 2045 | 728831 | !!!! ALU SUBFAMILY J WARNING ENTRY | 0.13 |
| 2046 | 1710216 | (U79260) unknown [Homo sapiens] | 0.005 |
| 2047 | 558401 | (Z38113) incomplete orf, len: 160, CAI: 0.09 similar to MRP_ECOLI | 1e−035 |
| | | P21590 39.9 KD PROTEIN | |
| 2048 | 224398 | ORF [Simian virus 40] | 1.6 |
| 2049 | 728838 | !!!! ALU SUBFAMILY SX WARNING ENTRY | 0.001 |
| 2050 | 728831 | !!!! ALU SUBFAMILY J WARNING ENTRY | 3e−005 |
| 2051 | 728831 | !!!! ALU SUBFAMILY J WARNING ENTRY | 3e−005 |
| 2052 | 2981631 | (AB012223) ORF2 [Canis familiaris] | 6e−007 |
| 2053 | 2981631 | (AB012223) ORF2 [Canis familiaris] | 3e−007 |
| 2054 | 2052393 | (U86758) netrin-2 like protein [Homo sapiens] sapiens] | 5e−012 |
| 2055 | 3023928 | PROBABLE HISTONE DEACETYLASE 1-2 | 6e−031 |
| 2056 | 2500285 | 60S RIBOSOMAL PROTEIN L5 | 0.015 |
| 2057 | 728836 | !!!! ALU SUBFAMILY SP WARNING ENTRY | 0.0003 |
| 2058 | 113668 | !!!! ALU CLASS C WARNING ENTRY !!!! | 0.001 |
| 2059 | 1546035 | (U64570) myelin/oligodendrocyte glycoprotein-16.3 kD(a) precursor | 0.0006 |
| | | [Homo sapiens] >gi\|1546037 (U64571) myelin/oligodendrocyte | |
| | | glycoprotein-16.3 kD(b) precursor | |
| 2060 | 543944 | CBID PROTEIN >gi\|154423 (L12006) putative [Salmonella | 5.9 |
| 2064 | 728836 | !!!! ALU SUBFAMILY SP WARNING ENTRY | 0.002 |
| 2065 | 728831 | !!!! ALU SUBFAMILY J WARNING ENTRY | 6e−006 |
| 2068 | 728831 | !!!! ALU SUBFAMILY J WARNING ENTRY | 0.013 |
| 2069 | 2072977 | (U93574) putative p150 [Homo sapiens] | 0.001 |
| 2070 | 2072972 | (U93572) putative p150 [Homo sapiens] | 8e−005 |
| 2071 | 130402 | RETROVIRUS-RELATED POL POLYPROTEIN | 4e−013 |
| 2072 | 728831 | !!!! ALU SUBFAMILY J WARNING ENTRY | 0.002 |
| 2073 | 728831 | !!!! ALU SUBFAMILY J WARNING ENTRY | 0.007 |
| 2074 | 728831 | !!!! ALU SUBFAMILY J WARNING ENTRY | 4e−006 |
| 2075 | 987050 | (X65335) lacZ [Cloning vector pSV-beta-Galactosidase Control] | 0.017 |
| 2076 | 106322 | hypothetical protein (L1H 3′ region) - human | 7e−006 |
| 2078 | 804808 | (M13100) unknown protein [Rattus norvegicus] | 0.11 |
| 2079 | 347964 | (L22453) TARBP-b gene product [Homo sapiens] | 0.001 |
| 2080 | 1196432 | (M22333) unknown protein [Homo sapiens] | 4e−005 |
| 2081 | 728831 | !!!! ALU SUBFAMILY J WARNING ENTRY | 8e−006 |
| 2082 | 3023928 | PROBABLE HISTONE DEACETYLASE 1-2 | 5e−037 |
| 2083 | 4505895 | pleiotropic regulator 1 (PRL1, Arabidopsis homolog) >gi\|2832296 | 6e−071 |
| | | (AF044333) pleiotropic regulator 1 [Homo sapiens] | |
| 2086 | 728836 | !!!! ALU SUBFAMILY SP WARNING ENTRY | 2e−005 |
| 2087 | 126296 | LINE-1 REVERSE TRANSCRIPTASE HOMOLOG protein | 6e−008 |
| | | [Nycticebus coucang] | |
| 2088 | 126296 | LINE-1 REVERSE TRANSCRIPTASE HOMOLOG protein | 7e−006 |
| | | [Nycticebus coucang] | |
| 2089 | 106322 | hypothetical protein (L1H 3′ region) - human | 2e−006 |
| 2090 | 2981631 | (AB012223) ORF2 [Canis familiaris] | 1e−014 |
| 2091 | 3336903 | (Y10809) bZIP DNA-binding protein | 2.1 |
| 2092 | 728831 | !!!! ALU SUBFAMILY J WARNING ENTRY | 0.01 |
| 2093 | 126296 | LINE-1 REVERSE TRANSCRIPTASE HOMOLOG protein | 0.002 |
| | | [Nycticebus coucang] | |
| 2094 | 728837 | !!!! ALU SUBFAMILY SQ WARNING ENTRY | 4e−008 |
| 2095 | 113671 | !!!! ALU CLASS F WARNING ENTRY !!!! | 0.0001 |
| 2096 | 386786 | (J04988) 90 kD heat shock protein [Homo sapiens] | 3e−009 |
| 2097 | 106322 | hypothetical protein (L1H 3′ region) - human | 7e−005 |
| 2099 | 126296 | LINE-1 REVERSE TRANSCRIPTASE HOMOLOG protein | 0.1 |
| | | [Nycticebus coucang] | |
| 2100 | 728831 | !!!! ALU SUBFAMILY J WARNING ENTRY | 1e−005 |
| 2101 | 2981631 | (AB012223) ORF2 [Canis familiaris] | 1e−006 |
| 2102 | 2500367 | 60S RIBOSOMAL PROTEIN L21 musculus] | 4e−007 |
| 2103 | 4106562 | (Z83819) dJ146H21.2 (similar to CYTOCHROME B-245 HEAVY | 2e−061 |
| | | CHAIN) [Homo sapiens] | |
| 2104 | 728831 | !!!! ALU SUBFAMILY J WARNING ENTRY | 8e−006 |
| 2106 | 1869835 | (Z86099) protein kinase [human herpesvirus 2] | 8.9 |
| 2107 | 1754989 | (U47661) proline-rich protein PRP2 precursor [Lupinus luteus] | 6.7 |
| 2108 | 2580578 | (AF000996) ubiquitous TPR motif, Y isoform [Homo sapiens] | 0.0004 |
| 2109 | 1173220 | 40S RIBOSOMAL PROTEIN S15A | 0.0003 |
| 2110 | 3646139 | (AJ011081) mader-drop8 [Homo sapiens] | 2e−006 |
| 2111 | 3192897 | (AF066071) SP85; PsB [Dictyostelium discoideum] | 1e−008 |
| 2112 | 126295 | LINE-1 REVERSE TRANSCRIPTASE HOMOLOG | 8e−009 |
| 2113 | 3599336 | (AF081110) ORF2 [Mus musculus domesticus] | 5e−012 |
| 2115 | 4185943 | (Y17833) pol protein [Human endogenous retrovirus K] | 2e−022 |
| 2118 | 53912 | (X57960) ribosomal protein L7 [Mus musculus] | 3e−027 |
| 2120 | 2506605 | HYPOTHETICAL 65.0 KD PROTEIN IN HUPB-COF | 4.5 |
| | | INTERGENIC REGION >gi\|1580716\|dbj\|BAA11649\|hypothetical | |
| 2121 | 2642222 | (AF030885) telomere-associated recQ-like helicase | 0.001 |
| 2125 | 1085573 | microtubule-associated protein MAP2 - rat molecular weight | 1.9 |
| | | microtubule-associated protein cell line, Peptide Partial, 381 aa] | |
| 2126 | 3192899 | (AF066072) SP85; PsB [Dictyostelium discoideum] | 0.024 |
| 2127 | 106322 | hypothetical protein (L1H 3′ region) - human | 1e−005 |
| 2128 | 2981631 | (AB012223) ORF2 [Canis familiaris] | 2e−010 |
| 2129 | 403460 | (L24521) transformation-related protein [Homo sapiens] | 0.003 |
| 2130 | 1082633 | Nascent polypeptide associated complex alpha chain - human | 3e−006 |
| | | >gi\|556642\|emb\|CAA56869\|(X80909) Nascent polypeptide | |
| | | associated complex alpha subunit [Homo sapiens] >gi\|4092060 | |
| | | (AF054187) alpha NAC [Homo sapiens] | |
| 2132 | 1669472 | (U53757) pol polyprotein [Feline immunodeficiency virus] | 6.2 |
| 2133 | 1127036 | (D26178) serine/threonine protein kinase | 0.81 |
| 2134 | 728835 | !!!! ALU SUBFAMILY SC WARNING ENTRY | 2e−007 |
| 2136 | 728837 | !!!! ALU SUBFAMILY SQ WARNING ENTRY | 5e−008 |
| 2137 | 730246 | OX-2 MEMBRANE GLYCOPROTEIN PRECURSOR precursor - | |
| | | human (fragment) >gi\|1335216\|emb\|CAA28943\| | 3e−008 |
| 2138 | 3341992 | (AF054174) histone macroH2A1.2 [Homo sapiens] | 6e−033 |
| 2139 | 1363325 | RNA helicase HEL117 - rat >gi\|897915 | 9.9 |
| 2140 | 3892705 | (AL033545) putative protein [Arabidopsis thaliana] | 0.14 |
| 2141 | 2565196 | (AF000381) non-functional folate binding protein [Homo sapiens] | 0.007 |
| 2142 | 106322 | hypothetical protein (L1H 3′ region) - human | 5e−007 |
| 2143 | 106322 | hypothetical protein (L1H 3′ region) - human | 0.033 |
| 2144 | 133902 | 40S RIBOSOMAL PROTEIN S27 ribosomal protein S27 isoform | 0.0004 |
| | | [Homo sapiens] | |
| 2145 | 111814 | hypothetical protein 3 - rat norvegicus] | 2e−011 |
| 2146 | 1669454 | (U53748) pol polyprotein [Feline immunodeficiency virus] | 8.6 |
| 2147 | 126296 | LINE-1 REVERSE TRANSCRIPTASE HOMOLOG protein | 6e−005 |
| | | [Nycticebus coucang] | |
| 2148 | 4506701 | ribosomal protein S23 S23 >gi\|543449\|pir\|\|S41955 ribosomal protein | 3e−009 |
| | | S23 - rat protein [Homo sapiens] >gi\|453281\|emb\|CAA54584\| | |
| | | (X77398) ribosomal protein S23 [Rattus norvegicus] | |
| 2149 | 126296 | LINE-1 REVERSE TRANSCRIPTASE HOMOLOG protein | 3e−011 |
| | | [Nycticebus coucang] | |
| 2152 | 1353390 | (U34998) Rad9 [Coprinus cinereus] | 8 |
| 2153 | 2134082 | hepatocyte growth factor receptor - African clawed frog | 4.8 |
| 2154 | 1196432 | (M22333) unknown protein [Homo sapiens] | 3e−006 |
| 2155 | 730451 | 60S RIBOSOMAL PROTEIN L13A (23 KD HIGHLY BASIC | 2e−008 |
| | | PROTEIN) >gi\|345897\|pir\|\|S29539 basic protein, 23K - human | |
| | | >gi\|23691\|emb\|CAA40254\|(X56932) 23 kD highly basic protein | |
| 2156 | 2072964 | (U93569) putative p150 [Homo sapiens] | 3e−009 |
| 2157 | 1791243 | (U83119) ORF2 consensus sequence encoding endonuclease and | 4e−012 |
| | | reverse transcriptase minus RNaseH [Rattus norvegicus] | |
| 2158 | 4506437 | retinoblastoma-binding protein 1; RBP 1 | 5e−008 |
| | | >gi\|1710030\|sp\|P29374\|RBB1_HUMAN RETINOBLASTOMA | |
| | | BINDING PROTEIN 1 (RBBP-1) >gi\|2136103\|pir\|\|I58383 | |
| | | retinoblastoma binding protein 1 - human protein 1, RBP1 [human, | |
| 2160 | 2981631 | (AB012223) ORF2 [Canis familiaris] | 0.0008 |
| 2161 | 126296 | LINE-1 REVERSE TRANSCRIPTASE HOMOLOG protein | 0.0005 |
| | | [Nycticebus coucang] | |
| 2162 | 728831 | !!!! ALU SUBFAMILY J WARNING ENTRY | 2e−007 |
| 2163 | 114977 | MEMBRANE-ASSOCIATED PROTEIN HEM-2 H19 protein - | 2e−012 |
| | | mouse (fragment) >gi\|51136\|emb\|CAA43693\| | |
| 2165 | 126296 | LINE-1 REVERSE TRANSCRIPTASE HOMOLOG protein | 6e−005 |
| | | [Nycticebus coucang] | |
| 2166 | 126296 | LINE-1 REVERSE TRANSCRIPTASE HOMOLOG protein | 1e−012 |
| | | [Nycticebus coucang] | |
| 2172 | 106323 | hypothetical protein (L1H 5′ region) - human | 9e−010 |
| 2173 | 126296 | LINE-1 REVERSE TRANSCRIPTASE HOMOLOG protein | 2e−013 |
| | | [Nycticebus coucang] | |
| 2174 | 126296 | LINE-1 REVERSE TRANSCRIPTASE HOMOLOG protein | 2e−013 |
| | | [Nycticebus coucang] | |
| 2175 | 695802 | (L39061) transcription factor SL1 [Homo sapiens] | 4e−014 |
| 2177 | 2072964 | (U93569) putative p150 [Homo sapiens] | 5e−007 |
| 2180 | 1085573 | microtubule-associated protein MAP2 - rat molecular weight | 3.8 |
| | | microtubule-associated protein cell line, Peptide Partial, 381 aa] | |
| 2181 | 104623 | collagen, corneal - chicken (fragment) | 0.097 |
| 2182 | 2981631 | (AB012223) ORF2 [Canis familiaris] | 1e−010 |
| 2183 | 2493820 | CYTOCHROME C OXIDASE POLYPEPTIDE I ornatipinnis] | 3e−011 |
| 2184 | 2494737 | HYPOTHETICAL 71.7 KD PROTEIN F52H3.2 IN | 3e−046 |
| | | CHROMOSOME II >gi\|3877390\|emb\|CAA91322\| | |
| 2185 | 1709997 | DNA REPAIR PROTEIN RAD18 pombe] | 0.64 |
| 2186 | 59977 | (Z14310) tripartite fusion transcript PLA2L | 1e−006 |
| 2187 | 2072967 | (U93570) putative p150 [Homo sapiens] | 5e−007 |
| 2188 | 1800307 | (U83883) p105 coactivator [Rattus norvegicus] | 4e−010 |
| 2189 | 728831 | !!!! ALU SUBFAMILY J WARNING ENTRY | 4e−009 |
| 2190 | 2245560 | (AF004339) cytochrome c oxidase subunit II [Homo sapiens] | 9e−007 |
| 2191 | 3875269 | (Z77655) Weak similarity to Human calcium-dependent proetase | 0.2 |
| | | (SW:CANS_HUMAN) | |
| 2192 | 728835 | !!!! ALU SUBFAMILY SC WARNING ENTRY | 2e−010 |
| 2193 | 106322 | hypothetical protein (L1H 3′ region) - human | 3e−013 |
| 2194 | 2072967 | (U93570) putative p150 [Homo sapiens] | 1e−015 |
| 2196 | 189086 | (M18728) ORF1 [Homo sapiens] | 0.0005 |
| 2197 | 2605776 | (AF027404) signal recognition particle 14a [Macaca radiata] | 0.0003 |
| 2202 | 339777 | (M80344) ORF2 contains a reverse transcriptase domain. | 5e−017 |
| 2203 | 4539386 | (AL035526) extensin-like protein | 1 |
| 2204 | 3043654 | (AB011137) KIAA0565 protein [Homo sapiens] | 3e−020 |
| 2206 | 3929396 | HYPOTHETICAL 47.8 KD PROTEIN F57B9.5 IN | 0.1 |
| | | CHROMOSOME III >gi\|532824 (U13876) F57B9.5 gene product | |
| 2207 | 1769472 | (U15780) p82 [Homo sapiens] | 9e−037 |
| 2208 | 728837 | !!!! ALU SUBFAMILY SQ WARNING ENTRY | 8e−015 |
| 2209 | 2506089 | 26S PROTEASE REGULATORY SUBUNIT 7 | 1e−016 |
| 2210 | 1073466 | aldehyde dehydrogenase (EC 1.2.1.-) aldB - Escherichia coli | 1.4 |
| | | >gi\|912476 (U00039) No definition line found [Escherichia coli] | |
| | | >gi\|1790014 (AE000436) aldehyde dehydrogenase B (lactaldehyde | |
| 2211 | 2072960 | (U93568) p40 [Homo sapiens] | 3e−005 |
| 2212 | 4009460 | (AF039401) calcium-dependent chloride channel-1 [Homo sapiens] | 2e−020 |
| 2214 | 100687 | hydroxyproline-rich glycoprotein - rice glycoprotein [Oryza sativa] | 8.7 |
| 2215 | 3043616 | (AB011118) KIAA0546 protein [Homo sapiens] | 2e−010 |
| 2216 | 126296 | LINE-1 REVERSE TRANSCRIPTASE HOMOLOG protein | 6e−014 |
| | | [Nycticebus coucang] | |
| 2218 | 91043 | LINE-1 hypothetical protein - mouse (fragment) musculus] | 3e−005 |
| 2220 | 4106562 | (Z83819) dJ146H21.2 (similar to CYTOCHROME B-245 HEAVY | 5e−061 |
| | | CHAIN) [Homo sapiens] | |
| 2221 | 631507 | zinc-containing protein - human | 0.0001 |
| 2223 | 2072972 | (U93572) putative p150 [Homo sapiens] | 3e−009 |
| 2224 | 3123174 | HYPOTHETICAL PROTEIN | 1e−012 |
| 2226 | 126296 | LINE-1 REVERSE TRANSCRIPTASE HOMOLOG protein | 1e−007 |
| | | [Nycticebus coucang] | |
| 2227 | 2981631 | (AB012223) ORF2 [Canis familiaris] | 3e−013 |
| 2228 | 109930 | GTP-binding protein rah - mouse (fragment) G-protein=low- | 4e−020 |
| | | molecular-weight GTP-binding protein GTP-binding protein [Mus | |
| 2229 | 2981631 | (AB012223) ORF2 [Canis familiaris] | 5e−007 |
| 2230 | 1698455 | (U49974) mariner transposase [Homo sapiens] | 5e−007 |
| 2231 | 2289235 | (U95016) myocyte nuclear factor-beta [Mus musculus] | 8e−015 |
| 2232 | 2827495 | (AL009196) 1-evidence=predicted by content; 1- | 1.9 |
| | | method=genefinder; 084; 1-evidence_end; 2-evidence=predicted by | |
| | | match; 2-match_accession=AA391048; 2- | |
| | | match_description=LD09991 .5prime LD Drosophila melanogaster | |
| 2233 | 2465330 | (U92818) unnamed HERV-H protein [Homo sapiens] | 1e−011 |
| 2234 | 2135837 | nuclear protein H731 - human >gi\|1825562 | 4e−014 |
| 2235 | 4164448 | (AF044958) NADH:ubiquinone oxidoreductase ASHI subunit | 2e−025 |
| 2236 | 4506077 | protein kinase C substrate 80 K-H | 4e−031 |
| | | >gi\|120629\|sp\|P14314\|G19P_HUMAN PROTEIN KINASE C | |
| | | SUBSTRATE, 80 KD PROTEIN, HEAVY CHAIN (PKCSH) (80 K- | |
| | | H PROTEIN) >gi\|105167\|pir\|\|A32469 80 K protein H precursor 80 K- | |
| 2237 | 2981631 | (AB012223) ORF2 [Canis familiaris] | 0.007 |
| 2238 | 1176422 | (U43194) rhophilin [Mus musculus] | 0.81 |
| 2239 | 4263743 | (AC004923) similar to UNC-93; similar to U89424 (PID:g3642687) | 3e−041 |
| | | [Homo sapiens] | |
| 2240 | 106322 | hypothetical protein (L1H 3′ region - human | 2e−018 |
| 2241 | 2352427 | (AF004161) peroxisomal Ca-dependent solute carrier | 1e−044 |
| 2242 | 4104400 | (AF035401) exocellobiohydrolase precursor [Piromyces rhizinflata] | 4.2 |
| 2243 | 158154 | (M81959) POU domain protein [Drosophila melanogaster] | 3 |
| 2244 | 114858 | MAGNESIUM-PROTOPORPHYRIN IX MONOMETHYL ESTER | 2.2 |
| | | OXIDATIVE CYCLASE 66 KD SUBUNIT Rhodobacter capsulatus | |
| | | >gi\|46113\|emb\|CAA77530\|(Z11165) 575 aa (66 kD) oxidative | |
| 2245 | 2352427 | (AF004161) peroxisomal Ca-dependent solute carrier | 3e−044 |
| 2246 | 116746 | PROBABLE COAT PROTEIN virus >gi\|58812\|emb\|CAA29526\| | 4.3 |
| | | (X06166) ORF IV (AA 1-489) | |
| 2247 | 2072967 | (U93570) putative p150 [Homo sapiens] | 6e−008 |
| 2248 | 2137494 | M-sema F protein precusor - mouse homolog=M-Sema F [mice, | 5.5 |
| | | neonatal brain, Peptide, 834 aa] | |
| 2249 | 1709971 | 60S RIBOSOMAL PROTEIN L10A (CSA-19) | 1e−015 |
| 2250 | 1498225 | (Y07569) PHAP12a protein [Homo sapiens] | 4e−012 |
| 2251 | 1711550 | TRANSLOCON-ASSOCIATED PROTEIN, DELTA SUBUNIT | 3e−012 |
| | | PRECURSOR (TRAP-DELTA) (SIGNAL SEQUENCE RECEPTOR | |
| | | DELTA SUBUNIT) (SSR-DELTA) unnamed protein product [Homo | |
| | | sapiens] >gi\|1302656 sapiens] >gi\|1673433\|emb\|CAA92215\| | |
| 2252 | 126296 | LINE-1 REVERSE TRANSCRIPTASE HOMOLOG protein | 1e−013 |
| | | [Nycticebus coucang] | |
| 2253 | 4502643 | chaperonin containing T-complex subunit 6 | 1e−014 |
| | | >gi\|730922\|sp\|P40227\|TCPZ_HUMAN T-COMPLEX PROTEIN 1, | |
| | | ZETA SUBUNIT (TCP-1-ZETA) (CCT-ZETA) (TCP20) sapiens] | |
| 2254 | 2737894 | (U59151) Cbf5p homolog [Homo sapiens] | 4e−070 |
| 2255 | 1076557 | extensin-like protein - cowpea (fragment) | 0.6 |
| 2257 | 3878245 | (Z69664) Similarity to Yeast cell divison control protein cdc25 | 9.2 |
| | | (SW:CC@%_SACKL); cDNA EST EMBL:D32475 comes from this | |
| | | gene; cDNA EST EMBL:D34376 comes from this gene; cDNA EST | |
| | | EMBL:D35124 comes from this gene; cDNA EST EMBL:D37... | |
| 2258 | 4505067 | MAD2 (mitotic arrest deficient, yeast, homolog)-like 1 >gi\|950199 | 2e−009 |
| | | (U31278) mitotic feedback control protein Madp2 homolog [Homo | |
| 2259 | 126296 | LINE-1 REVERSE TRANSCRIPTASE HOMOLOG protein | 4e−018 |
| | | [Nycticebus coucang] | |
| 2260 | 4507669 | tumor protein, translationally-controlled 1 CONTROLLED TUMOR | 0.043 |
| | | PROTEIN (TCTP) (P23) factor - human >gi\|37496\|emb\|CAA34200\| | |
| | | (X16064) tumor protein (AA 1-172) [Homo sapiens] | |
| 2261 | 66499 | arylamine N-acetyltransferase (EC 2.3.1.5), monomorphic - rabbit | 1.8 |
| | | >gi\|217728\|dbj\|BAA00989\|(D10108) arylamine N-acetyltransferase | |
| | | [Oryctolagus cuniculus] | |
| 2262 | 733532 | (U23420) unknown [Drosophila melanogaster] | 0.51 |
| 2263 | 3041664 | DEOXYURIDINE 5′-TRIPHOSPHATE | 2e−029 |
| | | NUCLEOTIDOHYDROLASE PRECURSOR (DUTPASE) (DUTP | |
| | | PYROPHOSPHATASE) >gi\|2443580 (AF018432) dUTPase [Homo | |
| | | sapiens] >gi\|2735292 (U90223) deoxyuridine triphosphate | |
| 2264 | 2136246 | tastin - human >gi\|905356 (U04810) tastin | 1.1 |
| 2266 | 126295 | LINE-1 REVERSE TRANSCRIPTASE HOMOLOG | 0.0002 |
| 2267 | 825671 | (X16934) B23 nucleophosmin (280 AA) [Homo sapiens] | 4e−013 |
| 2268 | 2231019 | (Z97207) B-IND1 protein [Mus musculus] | 2e−023 |
| 2269 | 4502189 | aquaporin 8 sapiens] | 1e−014 |
| 2270 | 126296 | LINE-1 REVERSE TRANSCRIPTASE HOMOLOG protein | 5e−015 |
| | | [Nycticebus coucang] | |
| 2271 | 126296 | LINE-1 REVERSE TRANSCRIPTASE HOMOLOG protein | 5e−015 |
| | | [Nycticebus coucang] | |
| 2272 | 218564 | (D90358) HB-SOD [Schizosaccharomyces pombe] | 4e−024 |
| 2274 | 1352736 | PELOTA PROTEIN >gi\|973224 (U27197) pelota [Drosophila | 3e−038 |
| 2275 | 102177 | hypothetical protein (ribosomal RNA repeat region) - Giardia lamblia | 1.3 |
| 2276 | 113668 | !!!! ALU CLASS C WARNING ENTRY !!!! | 0.13 |
| 2277 | 961444 | (D63876) KIAA0154 gene product is related to mouse gamma | 7e−026 |
| | | adaptin. [Homo sapiens] | |
| 2278 | 3183217 | HYPOTHETICAL PROTEIN KIAA0103 sapiens] | 5e−017 |
| 2282 | 2072972 | (U93572) putative p150 [Homo sapiens] | 4e−017 |
| 2283 | 961444 | (D63876) KIAA0154 gene product is related to mouse gamma | 6e−028 |
| | | adaptin. [Homo sapiens] | |
| 2285 | 4586287 | (AB004794) DUF140 [Xenopus laevis] | 0.005 |
| 2286 | 3879684 | (Z74042) predicted using Genefinder; Similarity to Haemophilus 3- | 0.0002 |
| | | oxoacyl-(acyl-carrier protein) reductase (SW:FABG_HAEIN); cDNA | |
| | | EST yk470b2.3 comes from this gene; cDNA EST yk470b2.5 comes | |
| | | from this gene [Caenorhabditis elegans] | |
| 2287 | 131786 | RAS-RELATED PROTEIN RAB-1A protein ypt1 - mouse | 1e−017 |
| | | >gi\|2144599\|pir\|\|TVHUYP GTP-binding protein Rab1 - human | |
| | | >gi\|2144600\|pir\|\|TVDGYP GTP-binding protein Rab1 - dog | |
| | | >gi\|55457\|emb\|CAA68284\|(Y00094) Ypt1 protein (AA 1-205) [Mus | |
| | | musculus] >gi\|550060 (M28209) GTP | |
| 2288 | 4115532 | (AB012043) NBR13 [Homo sapiens] | 0.69 |
| 2289 | 4586287 | (AB004794) DUF140 [Xenopus laevis] | 1e−007 |
| 2290 | 126296 | LINE-1 REVERSE TRANSCRIPTASE HOMOLOG protein | 9e−020 |
| | | [Nycticebus coucang] | |
| 2293 | 2981950 | Pathogenesis-Related Protein 5d From Nicotiana Tabacum | 7.8 |
| 2294 | 482300 | cell surface antigen CD34 precursor - human | 1e−019 |
| 2295 | 104623 | collagen, corneal - chicken (fragment) | 0.14 |
| 2296 | 4191746 | (L30113) alcohol dehydrogenase; ADH [Papio hamadryas] | 2e−018 |
| 2297 | 1518609 | (U57715) FGF receptor activating protein FRAG1 [Rattus | 1e−020 |
| 2298 | 4502953 | collagen, type IV, alpha 4 CHAIN PRECURSOR | 0.025 |
| | | >gi\|1360674\|pir\|\|CGHU1B collagen alpha 4(IV) chain precursor- | |
| | | human >gi\|574806\|emb\|CAA56943\|IV [Homo sapiens] | |
| 2299 | 2565196 | (AF000381) non-functional folate binding protein [Homo sapiens] | 1e−006 |
| 2300 | 2492620 | NEURONAL ACETYLCHOLINE RECEPTOR PROTEIN, ALPHA- | 1e−009 |
| | | 6 CHAIN PRECURSOR >gi\|1458118 (U62435) nicotinic | |
| | | acetylcholine receptor alpha6 subunit precursor [Homo sapiens] | |
| | | >gi\|2815225\|emb\|CAA76155\|precursor [Homo sapiens] | |
| 2301 | 95095 | hypothetical protein 2 - Agrobacterium tumefaciens | 3.3 |
| | | >gi\|39102\|emb\|CAA37890.1\|(X53945) ORF2 | |
| 2302 | 1765938 | (U47077) DNA-dependent protein kinase catalytic subunit | 6e−021 |
| 2303 | 2493783 | COLLAGEN ALPHA 4(IV) CHAIN bovine (fragment) | 3 |
| 2304 | 2645205 | (U63648) p160 myb-binding protein [Mus musculus] | 3e−029 |
| 2305 | 2645205 | (U63648) p160 myb-binding protein [Mus musculus] | 9e−030 |
| 2306 | 2495322 | HOMEOBOX PROTEIN HOX-A9 | 8e−014 |
| 2307 | 116509 | CALCYCLIN (PROLACTIN RECEPTOR ASSOCIATED | 4e−006 |
| | | PROTEIN) (PRA) (GROWTH FACTOR-INDUCIBLE PROTEIN | |
| | | 2A9) (S100 CALCIUM-BINDING PROTEIN A6) put. calcyclin; | |
| 2308 | 4502991 | cytochrome c oxidase subunit VIIb | 0.002 |
| | | >gi\|461804\|sp\|P24311\|COXM_HUMAN CYTOCHROME C | |
| | | OXIDASE POLYPEPTIDE VIIB PRECURSOR 1.9.3.1) chain VIIb- | |
| | | human >gi\|30151\|emb\|CAA78613\|sapiens] | |
| 2309 | 4507669 | tumor protein, translationally-controlled 1 CONTROLLED TUMOR | 6e−020 |
| | | PROTEIN (TCTP) (P23) factor - human >gi\|37496\|emb\|CAA34200\| | |
| | | (X16064) tumor protein (AA 1-172) [Homo sapiens] | |
| 2311 | 4507207 | sorcin sorcin [Homo sapiens] >gi\|1094394\|prf\|\|2106141A sorcin | 2e−018 |
| 2312 | 117061 | CYTOCHROME C OXIDASE POLYPEPTIDE III chain III - human | 3e−017 |
| | | mitochondrion(SGC1) oxidase III [Homo sapiens] | |
| 2313 | 1709972 | 60S RIBOSOMAL PROTEIN L10A (CSA-19) | 5e−013 |
| 2314 | 4454698 | (AF070661) HSPC005 [Homo sapiens] | 3e−014 |
| 2315 | 1885381 | (U77665) RNaseP protein P30 [Homo sapiens] | 1e−020 |
| 2316 | 209383 | (M27786) MS-2 pol-stefin B fusion protein [Artificial gene] | 7e−008 |
| 2318 | 631507 | zinc-containing protein - human | 0.03 |
| 2319 | 4502991 | cytochrome c oxidase subunit VIIb | 5e−013 |
| | | >gi\|461804\|sp\|P24311\|COXM_HUMAN CYTOCHROME C | |
| | | OXIDASE POLYPEPTIDE VIIB PRECURSOR 1.9.3.1) chain VIIb- | |
| | | human >gi\|30151\|emb\|CAA78613\|sapiens] | |
| 2320 | 87765 | hypothetical L1 protein (third intron of gene TS) - human | 2e−015 |
| | | >gi\|364964\|prf\|\|1510254A L1 repetitive element ORF [Homo | |
| 2321 | 129379 | MITOCHONDRIAL MATRIX PROTEIN P1 PRECURSOR (P60 | 8e−016 |
| | | LYMPHOCYTE PROTEIN) (60 KD CHAPERONIN) PROTEIN) | |
| | | (HUCHA60) >gi\|107086\|pir\|\|A32800 heat shock protein 60 precursor- | |
| | | human >gi\|190127 (M22382) mitochondrial matrix protein [Homo | |
| 2322 | 2231019 | (Z97207) B-IND1 protein [Mus musculus] | 6e−022 |
| 2323 | 4154176 | (U96639) ATPase subunit 6 [Canis familiaris] | 0.007 |
| 2324 | 2575807 | (D49692) adenylate cyclase [Spirulina platensis] | 4.4 |
| 2325 | 4502991 | cytochrome c oxidase subunit VIIb | 5e−007 |
| | | >gi\|461804\|sp\|P24311\|COXM_HUMAN CYTOCHROME C | |
| | | OXIDASE POLYPEPTIDE VIIB PRECURSOR 1.9.3.1) chain VIIb- | |
| | | human >gi\|30151\|emb\|CAA78613\|sapiens] | |
| 2326 | 2119918 | P43 - human >gi\|833999\|bbs\|160014 (S75463) P43=mitochondrial | 3e−020 |
| | | elongation factor homolog [human, liver, Peptide, 452 aa] [Homo | |
| 2327 | 4503145 | cathepsin E precursor - human >gi\|181194 (J05036) cathepsin E | 1e−041 |
| | | precursor [Homo sapiens] >gi\|181205 (M84424) cathepsin E | |
| 2328 | 4502189 | aquaporin 8 sapiens] | 2e−023 |
| 2329 | 4502189 | aquaporin 8 sapiens] | 2e−026 |
| 2330 | 2306969 | (AF007860) xl-Mago [Xenopus laevis] | 2e−037 |
| 2333 | 3970731 | (AJ006480) SBT4B protein [Lycopersicon esculentum] | 9 |
| 2334 | 2736524 | (AF039052) Similar to inosine monophosphate dehydrogenase; | 2.7 |
| | | coded for by C. elegans cDNA CEMSF04F; coded for by C. elegans | |
| | | cDNA yk247b12.3; coded for by C. elegans cDNA cm20d8; coded | |
| | | for by C. elegans cDNA yk247b12.5; coded for by C. elegans... | |
| 2335 | 1085957 | hypothetical protein Y - Streptomyces nogalater | 1.1 |
| | | >gi\|2147591\|pir\|\|S69232 hypothetical protein Y | |
| 2336 | 3183217 | HYPOTHETICAL PROTEIN KIAA0103 sapiens] | 3e−021 |
| 2337 | 2494312 | TRANSLATION INITIATION FACTOR EIF-2B GAMMA | 3e−037 |
| | | SUBUNIT (EIF-2B GDP-GTP EXCHANGE FACTOR) subunit | |
| 2338 | 337930 | (M22146) scar protein [Homo sapiens] | 1e−024 |
| 2339 | 68891 | transforming protein ets - chicken >gi\|211753 | 2e−007 |
| 2340 | 126296 | LINE-1 REVERSE TRANSCRIPTASE HOMOLOG protein | 9e−017 |
| | | [Nycticebus coucang] | |
| 2341 | 4507517 | tight junction protein 1 (zona occludens 1) | 9e−023 |
| | | >gi\|585098\|sp\|Q07157\|ZO1_HUMAN TIGHT JUNCTION | |
| | | PROTEIN ZO-1 (TIGHT JUNCTION PROTEIN 1) human | |
| | | >gi\|292938 (L14837) tight junction (zonula occludens) protein ZO-1 | |
| 2343 | 106851 | keratin 18, cytoskeletal - human (fragment) sapiens] | 4e−023 |
| 2344 | 1723438 | HYPOTHETICAL 52.3 KD PROTEIN C56F8.06C IN | 7.5 |
| | | CHROMOSOME I PRECURSOR | |
| 2345 | 2231019 | (Z97207) B-IND1 protein [Mus musculus] | 7e−022 |
| 2346 | 2047300 | (L40459) latent transforming growth factor-beta binding protein [Mus | 0.48 |
| | | musculus] | |
| 2347 | 129383 | PROBABLE RNA-DEPENDENT HELICASE P68 human | 2e−023 |
| | | >gi\|35220\|emb\|CAA36324\|(X52104) p68 protein (AA 1-614) [Homo | |
| | | sapiens] >gi\|38318\|emb\|CAA33751\|(X15729) protein p68 (AA 1- | |
| | | 614) [Homo sapiens] >gi\|2599360 | |
| 2351 | 4502411 | BCL2-interacting killer BCL-2 INTERACTING KILLER | 3e−025 |
| | | (APOPTOSIS INDUCER NBK) (BP4) sapiens] >gi\|1235989 | |
| 2352 | 2072961 | (U93568) putative p150 [Homo sapiens] | 2e−014 |
| 2354 | 2306969 | (AF007860) xl-Mago [Xenopus laevis] | 3e−041 |
| 2355 | 1872498 | (U74297) PiUS [Oryctolagus cuniculus] | 2e−033 |
| 2356 | 1783123 | (AB000170) endopeptidase 24.16 type M3 endopeptidase 24.16 type | 1e−029 |
| | | M3 [Sus scrofa] type M3 [Sus scrofa] >gi\|1783130\|dbj\|BAA19065\| | |
| | | type M3 [Sus scrofa] >gi\|1783134\|dbj\|BAA19067\|type M3 [Sus | |
| 2357 | 2306969 | (AF007860) xl-Mago [Xenopus laevis] | 2e−041 |
| 2358 | 1872498 | (U74297) PiUS [Oryctolagus cuniculus] | 6e−034 |
| 2359 | 4519602 | (AB017563) IGSF4 [Homo sapiens] | 3e−041 |
| 2360 | 1944330 | (D49545) KIFC2 [Mus musculus] | 2e−019 |
| 2361 | 4502189 | aquaporin 8 sapiens] | 6e−040 |
| 2362 | 4502189 | aquaporin 8 sapiens] | 2e−041 |
| 2363 | 1537070 | (U63840) nucleoporin p54 [Rattus norvegicus] | 2e−040 |
| 2364 | 3024124 | HOMEOBOX PROTEIN MEIS3 | 8e−030 |
| 2365 | 107215 | notch protein homolog TAN-1 precursor - human | 5e−040 |
| 2366 | 2306969 | (AF007860) xl-Mago [Xenopus laevis] | 7e−054 |
| 2367 | 2306969 | (AF007860) xl-Mago [Xenopus laevis] | 2e−054 |
| 2368 | 4464284 | (AC007136) Putative map kinase interacting kinase [Homo sapiens] | 7e−045 |
| | | [Homo sapiens] | |
| 2369 | 4502741 | cyclin-dependent kinase 6 KINASE 6 (KINASE PLSTIRE) | 1e−060 |
| | | >gi\|107662\|pir\|\|S23387 protein kinase (EC 2.7.1.37) cdk6 - human | |
| | | >gi\|4389286\|pdb\|1BI8\|A Chain A, Mechanism Of G1 Cyclin | |
| | | Dependent Kinase Inhibition From The Structures Cdk6-P19ink4d | |
| 2371 | 2496815 | HYPOTHETICAL 68.1 KD PROTEIN B0304.7 IN | 0.59 |
| | | CHROMOSOME II >gi\|1041881 (U39472) similar to f44f4.5 | |
| 2372 | 2496815 | HYPOTHETICAL 68.1 KD PROTEIN B0304.7 IN | 0.56 |
| | | CHROMOSOME II>gi\|1041881 (U39472) similar to f44f4.5 | |
| 2373 | 3327226 | (AB014606) KIAA0706 protein [Homo sapiens] | 1e−031 |
| 2374 | 3327226 | (AB014606) KIAA0706 protein [Homo sapiens] | 2e−034 |
| 2375 | 3327226 | (AB014606) KIAA0706 protein [Homo sapiens] | 2e−038 |
| 2376 | 2231019 | (Z97207) B-IND1 protein [Mus musculus] | 2e−039 |
| 2377 | 3327226 | (AB014606) KIAA0706 protein [Homo sapiens] | 4e−043 |
| 2378 | 417743 | NEUROLYSIN PRECURSOR (NEUROTENSIN | 1e−047 |
| | | ENDOPEPTIDASE) (MITOCHONDRIAL OLIGOPEPTIDASE M) | |
| | | ANGIOTENSIN-BINDING PROTEIN) (SABP) protein - pig | |
| | | >gi\|217709\|dbj\|BAA01949\|(D11336) soluble angiotesin-binding | |
| | | protein [Sus scrofa] type M1 [Sus scrofa] >gi\|1871389\|dbj\|BAA1 | |
| 2379 | 1783127 | (AB000172) endopeptidase 24.16 type M2 endopeptidase 24.16 type | 7e−050 |
| | | M2 [Sus scrofa] | |
| 2380 | 2494312 | TRANSLATION INITIATION FACTOR EIF-2B GAMMA | 7e−056 |
| | | SUBUNIT (EIF-2B GDP-GTP EXCHANGE FACTOR) subunit | |
| 2381 | 1085499 | CDC4 repeat unit-containing protein - mouse | 4e−056 |
| 2382 | 4092846 | (AB019987) chromosome-associated polypeptide-C [Homo sapiens] | 4e−058 |
| 2383 | 4103446 | (AF023265) NAD+-specific isocitrate dehydrogenase beta subunit | 5e−059 |
| | | isoform A [Homo sapiens] | |
| 2384 | 1353709 | (U42385) FIN16 gene product [Mus musculus] | 8e−061 |
| 2385 | 4106818 | (AF083395) phospholipase A2-activating protein [Homo sapiens] | 5e−061 |
| 2386 | 473407 | (U08215) NST-1 [Mus musculus] | 2e−061 |
| 2387 | 4103446 | (AF023265) NAD+- specific isocitrate dehydrogenase beta subunit | 6e−062 |
| | | isoform A [Homo sapiens] | |
| 2388 | 1537070 | (U63840) nucleoporin p54 [Rattus norvegicus] | 2e−062 |
| 2389 | 3851584 | (AF092563) chromosome-associated protein-E [Homo sapiens] | 7e−063 |
| 2390 | 3851584 | (AF092563) chromosome-associated protein-E [Homo sapiens] | 6e−063 |
| 2391 | 3493209 | (AF052577) aldo-keto reductase [Homo sapiens] | 3e−064 |
| 2392 | 4502741 | cyclin-dependent kinase 6 KINASE 6 (KINASE PLSTIRE) | 5e−065 |
| | | >gi\|107662\|pir\|\|S23387 protein kinase (EC 2.7.1.37) cdk6 - human | |
| | | >gi\|4389286\|pdb\|1BI8\|A Chain A, Mechanism Of G1 Cyclin | |
| | | Dependent Kinase Inhibition From The Structures Cdk6-P19ink4d | |
| 2393 | 3493209 | (AF052577) aldo-keto reductase [Homo sapiens] | 7e−066 |
| 2394 | 4165018 | (D89053) Acyl-CoA synthetase 3 [Homo sapiens] | 3e−068 |
| 2395 | 4106818 | (AF083395) phospholipase A2-activating protein [Homo sapiens] | 1e−068 |
| 2396 | 4165018 | (D89053) Acyl-CoA synthetase 3 [Homo sapiens] | 1e−070 |

TABLE 2A
Nearest Neighbor (BlastN vs. GenBank)

| SEQ ID | ACCESSION | DESCRIPTION | P VALUE | |
|---|---|---|---|---|
| 101 | U73106 | Liriodendron tulipifera high-pI laccase (LAC2-4) mRNA, | 4.9 | |
| 102 | M63897 | Bacillus thuringiensis insecticidal crystal protein (CryIF) | 4.8 | |
| | | gene, complete cds. | | |
| 103 | X75014 | M. musculus Phox2 mRNA for homeodomain protein | 4.4 | |
| 104 | X77320 | A. officinalis L. unknown mRNA | 4.1 | |
| 105 | M64982 | Human fibrinogen alpha chain gene, complete mRNAs. | 4.1 | |
| 106 | U28241 | Gallus gallus collapsin-3 mRNA, partial cds. | 4.1 | |
| 107 | M36860 | Human elastin mRNA, complete cds. | 4.1 | |
| 108 | X99641 | M. musculus mRNA for HP1 alpha protein | 4.1 | |
| 109 | X92557 | S. erythraea pccB, bcpA2, and orfX genes | 4 | |
| 110 | M21514 | Rinderpest virus fusion protein mRNA, complete cds. 4 | | |
| 111 | M64982 | Human fibrinogen alpha chain gene, complete mRNAs. | 4 | |
| 112 | D87218 | Trypanosoma cruzi mRNA, partial cds, clone:TcEST002 | 4 | |
| 113 | M27273 | E. coli rafA, rafB, and rafD genes encoding Alpha-D- | 3.9 | |
| | | galactosidase, raf-permease, and raf-invertase, complete cds. | | |
| 114 | U50065 | Caenorhabditis elegans cosmid T23A7. | 3.9 | |
| 115 | X76186 | Rinderpest virus (REOK vaccine) mRNA for matrix protein | 3.9 | |
| 116 | L05165 | Gorilla gorilla glycophorin-gene related sequence. | 3.9 | |
| 117 | M77212 | Apis koschevnikovi niltochondrial cytocbrome oxidase II | 3.9 | |
| | | gene, complete cds. | | |
| 118 | U64453 | Human ELK1 pseudogene (ELK2) and immunoglobulin | 3.9 | |
| | | heavy chain gamma pseudogene (IGHGP) | | |
| 119 | M87502 | Rotavirus SA114F nonstructural protein 34 (NS34) mRNA, | 3.9 | |
| | | complete cds. | | |
| 120 | L24376 | Bacillus subtilis ribosomal protein L7/12 (rplL) gene, beta | 3.9 | |
| | | subunit of RNA polymerase (rpoBC) gene, 3′ end, complete | | |
| 121 | U97143 | Rattus norvegicus RET ligand 2 (RETL2) mRNA, complete | 3.9 | |
| 122 | M95610 | Human alpha 2 type IX collagen (COL9A2) mRNA, partial | 3.9 | |
| 123 | M21890 | Human alpha-1-antitrypsin like (PIL) pseudogene, exon 2. | 3.9 | |
| 124 | NM_002205.1 | Homo sapiens integrin, alpha 5 (fibronectin receptor, alpha | 3.9 | |
| | | polypeptide) (ITGA5) mRNA > ::emb\|X062S6.1\|HSFNRA | | |
| | | Human mRNA for integrin alpha 5 subunit | | |
| 125 | U38949 | Gallus gallus C-protein mRNA, complete cds. | 3.9 | |
| 126 | U04985 | Simian immunodeficiency virus clone SIVsm62J vpx gene, | 3.9 | |
| | | partial cds, env, nef, tat and rev genes, complete cds. | | |
| 127 | U05237 | Human fetal Alz-50-reactive clone 1 (FAC1) mRNA, | 3.9 | |
| 128 | X96616 | P. primaurelia gene encoding 156D surface antigen | 3.8 | |
| 129 | U31929 | Human orphan nuclear receptor (DAX1) gene, complete cds | 3.8 | |
| 130 | X13523 | Yeast CBS2 gene for cytocbrome b translational activator | 3.8 | |
| 131 | M57769 | Chinese vaccinia virus I segment DNA fragment. | 3.8 | |
| 132 | U08443 | HIV-1 isolate 652 clone 11 from Haiti, envelope | 3.8 | |
| | | glycoprotein partial cds. | | |
| 133 | L39876 | Caldicellulosiruptor saccharolyticus alpha-dextrin 6- | 3.8 | |
| | | glucanohydrolase (pu1A) and pepX genes, complete cds and | | |
| 134 | M20363 | Soybean heat-shock protein (Gmhsp26-A) gene, complete | 3.8 | |
| 135 | J02836 | Mouse beta-glucuronidase gene, complete cds.. | 3.8 | |
| 136 | U64880 | Eubacterium thermomarinus ribonuclease P RNA | 3.8 | |
| 137 | X93520 | E. caballus microsatellite DNA marker (clone ASB6) | 3.8 | |
| 138 | Z83151 | H. sapiens Fanconi anaemia group A gene, exon 6 | 3.8 | |
| 139 | D28484 | Aspergillus oryzae pgkA gene for phosphoglycerate kinase, | 3.8 | |
| | | complete cds > ::dbj\|E04898\|E04898 gDNA encoding | | |
| 140 | Z28091 | S. cerevisiae chromosome XI reading frame ORF YKL091c | 3.8 | |
| 141 | V01291 | Yeast gene for alcohol dehydrogenase | 3.8 | |
| 142 | U44843 | Lactococcus lactis plasmid pND324, complete sequence | 3.8 | |
| 143 | M13496 | Chicken type X collagen gene. | 3.8 | |
| 144 | Y14946 | Homo sapiens mRNA for SPIN protein | 3.8 | |
| 145 | U94776 | Human muscle glycogen phosphorylase (PYGM) gene, | 3.8 | |
| | | exons 6 through 17 | | |
| 146 | U43844 | Mus musculus cyclin D3 gene, complete cds | 3.8 | |
| 147 | X03431 | Drosophila melanogaster transposable element 297 | 3.8 | |
| 148 | U05237 | Human fetal Alz-50-reactive clone 1 (FAC1) mRNA, | 3.8 | |
| 149 | U92856 | Comptonia peregrina maturase (matK) gene, chloroplast | 3.8 | |
| | | gene encoding chloroplast protein, complete cds | | |
| 150 | X94165 | Human papillomavirus type 73 E6, E7, E1, E2, E4, L2, and | 3.7 | |
| 151 | U47875 | Drosophila azteca NDSSC 14012-0171.6 glycerolphosphate | 3.7 | |
| | | dehydrogenase (Gpd) gene, partial cds | | |
| 152 | X02882 | Human HLA class II alpha chain gene DZ-alpha | 3.7 | |
| 153 | AF005932 | Clavispora opuntiae Spt3 (SPT3) gene, complete cds | 3.7 | |
| 154 | Z11840 | D. melanogaster hedgehog gene DNA | 3.7 | |
| 155 | U06745 | Arabidopsis thaliana ecotype Landsberg K+ transport system | 3.7 | |
| | | AKT1 gene, complete cds. | | |
| 156 | U63362 | Unidentified crenarchaeote 16S ribosomal RNA gene, 5′ | 3.7 | |
| | | partial sequence | | |
| 157 | D30810 | Wheat gene for transcription factor HBP-1b(c38), final | 3.7 | |
| | | exon, partial cds | | |
| 158 | X56089 | X. laevis mRNA for alpha-subunit of G-protein, type G- | 3.7 | |
| 159 | X07701 | Chironomus tentans Balbiani ring mRNA BR 2.1 3′-end | 3.7 | |
| 160 | X64649 | G. gallus mRNA for restrictin | 3.7 | |
| 161 | Y13426 | Homo sapiens TCRDV2 gene, partial | 3.7 | |
| 162 | Y14443 | Homo sapiens mRNA for zinc finger protein | 3.7 | |
| 163 | U92794 | Mus musculus alpha glucosidase II beta subunit m RNA, | 3.7 | |
| 164 | Y09480 | A. europaeus genes encoding dehydrogenase and cytochrome | 3.7 | |
| 165 | NM_001659.1 | Homo sapiens ADP-ribosylation factor 3 (ARF3) mRNA> | 3.7 | |
| | | gb\|M74491\|HUMADPRF3A Human ADP-ribosylation | | |
| | | factor 3 mRNA, complete cds. | | |
| 166 | L20893 | Rice yellow mottle virus complete genome. | 3.7 | |
| 167 | AF019759 | Canis familiaris beta-glucuronidase (GUSB) mRNA, | 3.7 | |
| 168 | U62587 | Cricetulus griseus beta-1,6-N-acetylglucosaminyltransferase | 3.7 | |
| | | Lec4A cell line point mutant mRNA, complete cds | | |
| 169 | D50085 | Cucumis sativus mRNA for NADPH-protocblorophyllide | 3.7 | |
| | | oxidoreductase, complete cds | | |
| 170 | M81890 | Human interleukin 11 (IL11) gene, complete mRNA. | 3.7 | |
| 171 | M57765 | Human interleukin 11 mRNA, complete cds. | 3.7 | |
| 172 | X55880 | T. reesei ura3 (OMPdecase) gene for orotidine-5′-phosphate | 3.6 | |
| | | decarboxylase (EC 4.1.1.23) | | |
| 173 | J03028 | P. falciparum dihydrofolate reductase-thymidylate synthase | 3.6 | |
| | | gene, complete cds. | | |
| 174 | AF000949 | Canis familiaris keratin (KRT9) gene, complete cds | 3.6 | |
| 175 | U78718 | Dugesia tigrina 26S ribosomal RNA gene, partial sequence | 3.6 | |
| 176 | D16471 | Human mRNA, Xq terminal portion | 3.6 | |
| 177 | X69838 | H. sapiens mRNA for G9a | 3.6 | |
| 178 | M24685 | Human angiotensinogen (AGT) gene, exon 1 | 3.6 | |
| 179 | L05468 | Trichomonas vaginalis beta-tubulin (btub 1) gene, complete | 3.6 | |
| 180 | Y08926 | P. falciparum mRNA for AARP1 protein, partial | 3.6 | |
| 181 | M59743 | Rabbit cardiac muscle Ca-2+ release channel | 3.6 | |
| 182 | X51952 | Human UCP gene for uncoupling protein exons 1 and 2 | 3.6 | |
| 183 | U25180 | Candida albicans phosphoglycerate kinase (PGK1) gene, | 3.6 | |
| | | complete cds. | | |
| 184 | X58080 | Maize chioroplast ORF170 and psaA gene | 3.6 | |
| 185 | Z24609 | H. sapiens (D15502) DNA segment containing (CA) repeat; | 3.6 | |
| | | clone AFM361td9; single read | | |
| 186 | M24936 | Mus musculus (BALB/c) L1 repeat insertion in the kappa | 3.6 | |
| | | chain 5′ flank. | | |
| 187 | U50951 | Thermoanaerobacterium thermosulfurigenes orfA gene, | 3.6 | |
| | | partial cds, polygalacturonase precursor (pg1A), abcA, abcB | | |
| | | and sigma factor (sigA) genes, complete cds | | |
| 188 | U57999 | Mus musculus prosaposin (psap/SGP-1) gene, complete cds. | 3.6 | |
| 189 | AF000949 | Canis familiaris keratin (KRT9) gene, complete cds | 3.6 | |
| 190 | S54325 | nucleoprotein [tomato chlorotic spot virus, isolate BR-03, | 3.6 | |
| | | Genomic RNA, 292 nt] | | |
| 191 | S70572 | {endogenous retrovirus SY-3, provirus}[human, | 3.6 | |
| | | lymphocytes, Genomic, 2189 nt] | | |
| 192 | AE000092 | Rhizobium sp. NGR234 plasmid pNGR234a, section 29 of | 3.6 | |
| | | 46 of the complete plasmid sequence | | |
| 193 | U75285 | Homo sapiens apoptosis inhibitor survivin gene, | 3.6 | |
| 194 | X91404 | W. mirabilis mRNA for phosphoenolpyruvate carboxylase | 3.6 | |
| 195 | M17376 | Mouse alpha-1-acid glycoprotein I (AGP-1) gene, complete | 3.6 | |
| 196 | Z73360 | Human DNA sequence from cosmid 92M18, BRCA2 gene | 3.6 | |
| | | region chromosome 13q12-13 | | |
| 197 | L05364 | Arabidopsis thaliana polyubiquitin (ubq7) gene sequence. | 3.6 | |
| 198 | J04353 | Human papillomavirus type 31 (HPV-31) complete genome. | 3.6 | |
| 199 | X03882 | Paramecium primaurelia gene for G surface protein | 3.6 | |
| 200 | U53152 | Caenorhabditis elegans cosmid K11D5. | 3.6 | |
| 201 | M65126 | Human snRNP E protein pseudogene EB. | 3.6 | |
| 202 | J04186 | Yeast (S. cerevisiae) lysyl-tRNA synthetase (KRS1) alpha-2 | 3.6 | |
| | | subunit gene, complete cds. | | |
| 203 | L35281 | Mus musculus (clone MKT6) morphogenetic protein 1 | 3.6 | |
| | | (BMP-1/His), alternative splice | | |
| 204 | D83390 | Gallus gallus mRNA for connectin/titin, partial cds | 3.6 | |
| 205 | U22103 | Glycine max partial SIRE-1 sequence gag-protease | 3.6 | |
| | | polyprotein mRNA, complete cds | | |
| 206 | U82705 | Human interferon alpha2 upstream scaffold associated | 3.6 | |
| | | region 3 (SAR3) and non-SAR region DNA | | |
| 207 | U39389 | Rhagolefis cerasi 16S ribosomal RNA gene, mitochondrial | 3.5 | |
| | | gene encoding mitochondrial RNA, partial sequence | | |
| 208 | X13287 | Medicago sativa nodulin-25 gene | 3.5 | |
| 209 | X91337 | H. sapiens La/SS-B pseudogene 2 | 3.5 | |
| 210 | M19684 | Human alpha-1-antitrypsin-related protein gene, exons 3, 4 | 3.5 | |
| 211 | M35296 | Human tyrosine kinase arg gene mRNA. | 3.5 | |
| 212 | X88000 | S. tenacellus mRNA for ubiquinol:cytochrome c | 3.5 | |
| 213 | M61906 | Human P13-kinase associated p85 mRNA sequence. | 3.5 | |
| 214 | Z29084 | C. butyricum transposon containing tbcC gene | 3.5 | |
| 215 | M92039 | Gallus gallus violet sensitive cone opsin mRNA, complete | 3.5 | |
| 216 | D86478 | Schizosaccharornyces pombe DNA for Crb2, complete cds | 3.5 | |
| 217 | U35737 | Saccharomyces cerevisiae nuclear polyadenylated RNA- | 3.5 | |
| | | protein (NAB4) gene, complete cds. | | |
| 218 | M22860 | B. thuringiensis 20 and 67 kd mosquitocidal protein genes, | 3.5 | |
| | | complete cds and IS231-like transposase, 3′ end. | | |
| 219 | Z57857 | H. sapiens CpG island DNA genomic Mse1 fragment, clone | 3.5 | |
| | | 1d10, forward read cpg1d10.fla | | |
| 220 | U07059 | Pneumocystis carinii clone PC14103 major surface | 3.5 | |
| | | glycoprotein mRNA, complete cds. | | |
| 221 | X52978 | H. sapiens gene for lipoprotein lipase | 3.5 | |
| 222 | M24566 | Dictyostelium discoideum tRNA-Glu-GAA gene, clone | 3.5 | |
| 223 | U11058 | Homo sapiens calcium dependent potassium channel alpha | 3.5 | |
| | | subunit (MaxiK) mRNA, complete cds | | |
| 224 | M21514 | Rinderpest virus fusion rotein mRNA, complete cds. | 3.5 | |
| 225 | X80454 | HIV type 1 (CA7) env-gene | 3.5 | |
| 226 | X98695 | Bacteriophage T4 modA and modB genes | 3.5 | |
| 227 | X76186 | Rinderpest virus (RBOK vaccine) mRNA for protein | 3.5 | |
| 228 | S82456 | PGHS-2 = prostaglandin endoperoxide H synthase-2 | 3.5 | |
| 229 | D85194 | Arabidopsis thaliana mRNA, partial cds | 3.5 | |
| 230 | L35661 | Homo sapiens (subclone H8 4_b9 from P1 35 H5 C8) DNA | 3.5 | |
| 231 | L76205 | Colletotrichum gloeosporioides non-LTR retrotransposon | 3.5 | |
| 232 | NM_000464.1 | Homo sapiens xeroderma pigmentosum, complementation | 3.5 | |
| | | group F (XPF) mRNA > ::gb\|U64315\|HSU64315 Human | | |
| | | DNA repair endonuclease subunit | | |
| 233 | X02155 | Bovine mRNA fragment for thyroglobulin N-terminal region | 3.5 | |
| 234 | D16437 | Synechococcus sp. DNA for PacS, complete cds | 3.5 | |
| 235 | X97570 | Z. mays dek34 gene | 3.5 | |
| 236 | S65225 | PEP-19 = neuron-specific [mice, Genomic, 1358 nt, segment | 3.5 | |
| 237 | U33099 | Human immunodeficiency virus type 1 isolate GM4, | 3.5 | |
| | | envelope glycoprotein (env) gene, V1-V5 region, partial cds | | |
| 238 | X71604 | H. sapiens son-pseudogene | 3.5 | |
| 239 | L47357 | Homo sapiens TIMP1 gene, exon 1. | 3.5 | |
| 240 | AF015490 | Bos taurus immunoglobulin variable region mRNA, partial | 3.4 | |
| | | cds > :: gb\|AF015492\|AF015492 Bos taurus | | |
| | | immunoglobulin variable region mRNA, partial cds | | |
| 241 | U93308 | Arabidopsis thaliana decoy (DECOY) gene, complete cds | 3.4 | |
| | | and valyl tRNA synthetase (valRS) gene, partial cds | | |
| 242 | Y12576 | Arabidopsis thaliana mRNA for histone H2B | 3.4 | |
| 243 | X07977 | Aspergillus amstelodami mtDNA with ARS element | 3.4 | |
| 244 | X70276 | Yeast centromere-containing shuttle vector YCp50 | 3.4 | |
| 245 | J03268 | Yeast (S. cerevisiae) polymerase I gene, complete cds. | 3.4 | |
| 246 | AF013168 | Homo sapiens hamartin (TSC1) mRNA, complete cds | 3.4 | |
| 247 | M32476 | Rat carcinoembryonic antigen-related protein | 3.4 | |
| 248 | X83390 | Albinaria coerulea complete mithochondria DNA | 3.4 | |
| 249 | U63337 | Mus musculus cyclin-dependent kinase-2 alpha | 3.4 | |
| 250 | D85530 | Human CpG island sequence, clone G0310 | 3.4 | |
| 251 | M92423 | Human FK506-binding protein 12 (FKBP12) mRNA, exons | 3.4 | |
| | | 3 and 4, complete FKBP12A mRNA and complete cds. | | |
| 252 | AB002693 | Mouse mRNA for ISBT, complete cds | 3.4 | |
| 253 | L01057 | Human (clone TRI-6) satellite I repeat region. | 3.4 | |
| 254 | U11270 | Human antithrombin III gene, exon 1 and partial cds. | 3.4 | |
| 255 | AB000280 | Rattus norvegicus mRNA for peptide/histidine transporter, | 3.4 | |
| | | complete cds | | |
| 256 | J03886 | Rat skeletal muscle myosin light chain kinase, complete cds. | 3.4 | |
| 257 | M16809 | Yeast (S. cerevisiae) CLS4 gene encoding a Ca regulatory | 3.4 | |
| | | protein, complete cds. | | |
| 258 | X58286 | Drosophila genes z600, gdl, Eip28/29 and mex1 | 3.4 | |
| 259 | X07267 | Rat gene 33 5′-region | 3.4 | |
| 260 | Z68129 | H. sapiens IDH gamma gene and TRAP delta gene | 3.4 | |
| 261 | X87241 | H. sapiens mRNA for hFat protein | 3.4 | |
| 262 | Z93650 | O. bellus 28S rRNA gene, D2 variable region | 3.4 | |
| 263 | D87471 | Mus musculus mRNA for gsg3, complete cds | 3.4 | |
| 264 | L34193 | Ipomopsis aggregata chloroplast maturase | 3.4 | |
| 265 | AF012899 | Sambucus nigra ribosome inactivating protein precursor | 3.4 | |
| | | mRNA, complete cds | | |
| 266 | X17318 | Zea mays chioroplast trnC gene, rpoB gene, rpoC1 gene, | 3.4 | |
| | | rpoC2 gene and rps2 gene for transfer RNA-Cys, RNA | | |
| | | polymerase subunits beta, beta-1, beta-2 and ribosomal | | |
| 267 | L35661 | Homo sapiens (subclone H8 4_b9 from P1 35 H5 C8) DNA | 3.4 | |
| 268 | U25236 | Human immunodeficiency virus type 1 clone XSH159D1D | 3.4 | |
| | | envelope glycoprotein (env) pseudogene, v1-v5 region, | | |
| 269 | L81392 | Homo sapiens (subclone 1_c12 from P1 H39) DNA | 3.4 | |
| 270 | M60172 | G. domesticus novel collagen protein mRNA, 3′ end. | 3.4 | |
| 271 | M15263 | E. coli araBAD operon encoding L-ribulokinase, L-arabinose | 3.4 | |
| | | isomerase, and L-ribulose 5-phosphate 4-epimerase. | | |
| 272 | S53273 | OMP = olfactory marker protein {promoter} [mice, Genomic, | 3.4 | |
| 273 | U22056 | Mus musculus fertilin alpha precursor (ADAM 1) mRNA, | 3.3 | |
| 274 | X63382 | Antithamnion sp. rhodoplast genes atpI, atpH, atpG, atpF, | 3.3 | |
| | | atpD, atpA, orf1, orf2 and orf3 | | |
| 275 | U67462 | Methanococcus jannaschii section 4 of 150 of the complete | 3.3 | |
| 276 | M24566 | Dictyostelium discoideum tRiNA-Glu-GAA gene, clone | 3.3 | |
| 277 | L13609 | Human catalase (CAT) gene, exon 1, 5′ end. | | |
| 278 | Z11486 | Pinus strobus L. mRNA for pine globulin-1 | 3.3 | |
| 279 | X03366 | Bovine spleen trypsin inhibitor II (SI) gene | 3.3 | |
| 280 | D49558 | Human DNA for gastric inhibitory polypeptide receptor, | 3.3 | |
| | | exon 5, 6, 7, 8, 9, 10, 11 and 12 | | |
| 281 | U52110 | Sulfolobus solfataricus putative ribokinase and Dbh genes, | 3.3 | |
| | | complete cds, and putative ATPase gene, partial cds | | |
| 282 | M34663 | Human chaperonin (HSP60) non-functional pseudogene 4. | 3.3 | |
| 283 | X82303 | P. groenlandica mitochondrial cytochrome b gene | 3.3 | |
| 284 | D78174 | Mouse cerebellum mRNA for Zic4 protein, complete cds | 3.3 | |
| 285 | D86966 | Human mRNA for KIAA0211 gene, complete cds | 3.3 | |
| 286 | L13198 | Clortridium perfringens type B beta-toxin gene, complete | 3.3 | |
| 287 | J05516 | E. coli leucine-specific transport (LS-BP; LIV-BP) system | 3.3 | |
| | | (livHMGF) genes, complete cds. | | |
| 288 | M58318 | Homo sapiens ala gene. | 3.3 | |
| 289 | X57297 | A. majus TAM1 gene for TNP1 and TNP2 | 3.3 | |
| 290 | U33099 | Human immunodeficiency virus type 1 isolate GM4, | 3.3 | |
| | | envelope glycoprotein (env) gene, V1-V5 region, partial cds | | |
| 291 | D29809 | Coptis japonica mRNA for S-adenosyl-L- | 3.3 | |
| | | methionine:scoulerine 9-O-methyltransferase, complete cds | | |
| 292 | M12727 | Human T-cell surface antigen T3 delta-chain gene, exons | 3.3 | |
| | | 2, 3, 4 and 5, clone pKR-1. | | |
| 293 | X54601 | Human MFD72 dinucleotide repeat DNA >:: | 3.2 | |
| | | gb\|I31132\|I31132 Sequence 44 from U.S. Pat. No. 5582979 | 3.2 | |
| 294 | Y11740 | H. sapiens whn gene, exon 1a and 1b | 3.2 | |
| 295 | M24566 | Dictyostelium discoideum tRNA-Glu-GAA gene, clone | 3.2 | |
| 296 | S83358 | focal adhesion kinase/pp125FAK/FAK + {altematively | 3.2 | |
| | | spliced} [rats, striatum, mRNA, 4575 nt] | | |
| 297 | M68519 | Human pulmonary surfactant-associated protein SP-A | 3.2 | |
| | | (SFTP1) gene, complete cds. | | |
| 298 | U22056 | Mus musculus fertilin alpha precursor (ADAM 1) mRNA, | 3.2 | |
| 299 | AB005803.1 | Homo sapiens DNA for histidine-rich glycoprotein, | 3.2 | |
| 300 | M24566 | Dictyostelium discoideum tRNA-Glu-GAA gene, clone | 3.2 | |
| 301 | X66139 | M. fascicularis mRNA for epididymal apical protein I | 3.2 | |
| 302 | U16955 | Plasmodium falciparum ATPase 2 gene, complete cds. | 3.2 | |
| 303 | M87108 | Human immunodeficiency virus type 2 (FOPOLC4) | 3.2 | |
| | | polymerase fragment. | | |
| 304 | U67585 | Methanococcus jannaschii section 127 of 150 of the | 3.2 | |
| 305 | U70559 | Saccharomyces cerevisiae DNA repair/transcription protein | 3.2 | |
| | | Mms19p (MMS19) gene, complete cds | | |
| 306 | D88191 | Chlorella Virus vChta-1 gene, complete cds | 3.2 | |
| 307 | M32352 | Mouse renin (Ren-1-d) gene, complete cds. | 3.2 | |
| 308 | X64406 | Marburg Virus RNA for ORF-2 and ORF-3 | 3.2 | |
| 309 | NM_002763.1 | Homo sapiens prospero-related homeobox 1 protein (Prox 1) | 3.2 | |
| | | mRNA, complete cds | | |
| 310 | Y11681 | Homo sapiens for mitochondrial ribosomal protein | 3.2 | |
| 311 | S80986 | svp[40]= svp-related nuclear receptor/retinoid signaling | 3.2 | |
| | | modulator [zebrafishes, mRNA, 3876 nt] | | |
| 312 | Z18630 | R. norvegicus mRNA for J1-160/180 neural recognition | 3.2 | |
| 313 | AF012899 | Sambucus nigra ribosome inactivating protein precursor | 3.2 | |
| | | mRNA, complete cds | | |
| 314 | X14336 | Filamentous Bacteriophage I2-2 genome | 3.1 | |
| 315 | U58884 | Mus musculus SH3-containing protein SH3P7 mRNA, | 3.1 | |
| | | complete cds. similar to Human Drebrin | | |
| 316 | X66139 | M. fascicularis mRNA for epididymal apical protein I | 3.1 | |
| 317 | D90819 | E. coli genomic DNA, Kohara clone #328(39.4-39.8 min.) | 3.1 | |
| 318 | U78770 | Mus musculus spasmolytic polypeptide (mSP) gene, | 3.1 | |
| 319 | U06083 | Human N-acetylgalactosamine 6-sulphatase | 3.1 | |
| 320 | U48228 | Plasmodium falciparum ribosomal RNA gene, partial | 3 | |
| | | sequence, internal transcribed spacer 2, and large subunit | | |
| | | ribosomal RNA gene, complete sequence | | |
| 321 | X95188 | R. norvegicus mRNA for Pristanoyl:CoA Oxidase | 3 | |
| 322 | Z34932 | S. scrofa mRNA for protein phosphatase 2A 55 kDaf | 3 | |
| | | regulatory subunit, alpha isoform (partial) | | |
| 323 | U40837 | Ovine adenovirus terminal protein gene, partial cds, 52/55 K | 3 | |
| | | pIIIa, III, pVII, pX, pVI, II, endopeptidase and DNA | | |
| | | binding protein genes, complete cds, and 100 K hexon | | |
| 324 | X77233 | L. esculentum (de Ruiter 83G38) Adb2 gene | 2.9 | |
| 325 | M81341 | Plasmodium falciparum cysteine proteinase gene, complete | 2.8 | |
| 326 | X99832 | H. sapiens CLN3 gene, complete CDS | 2.7 | |
| 327 | M64703 | N. crassa valyl-tRNA synthetase (cyt-20/un-3) gene. | 2.1 | |
| 328 | AB001635.1 | Homo sapiens DNA for cGMP-binding cGMP-specific | 1.9 | |
| | | phosphodiesterase (PDE5), exon 21 and complete cds | | |
| 329 | X12669 | H. sapiens gene for arginase exon 8 and 3′-flanking region | 1.8 | |
| 330 | M19238 | Yeast (S. cerevisiae) ribosomal protein L44′ gene, complete | 1.8 | |
| 331 | AE001665 | Chlamydia pneumoniae section 81 of 103 of the complete | 1.8 | |
| 332 | X15603 | Human elastin gene, exon 1 | 1.8 | |
| 333 | AE000553.1 | Helicobacter pylori 26695 section 31 of 134 of the complete | 1.8 | |
| 334 | AB022333 | obligately oligotrophic bacteria POC-111 DNA for 16S | 1.8 | |
| | | rRNA, partial sequence | | |
| 335 | X51666 | S. cerevisiae DNA for SEC62 gene | 1.8 | |
| 336 | X16588 | B. nigra repeat DNA (clone pBN 35) | 1.8 | |
| 337 | U19253 | Xenopus laevis/gilli complement component C3 mRNA, | 1.8 | |
| 338 | U32770 | Haemophilus influenzae Rd section 85 of 163 of the | 1.7 | |
| 339 | U64618 | Propithecus verreauxi epsilon globin gene, 5′flanking region | 1.7 | |
| | | and exons 1-3, complete cds | | |
| 340 | U39700 | Mycoplasma genitalium section 22 of 51 of the complete | 1.7 | |
| 341 | Z82656 | R. prowazekii genomic DNA fragment (clone A45F) | 1.7 | |
| 342 | AL049337.1 | Homo sapiens mRNA; cDNA DKFZp564P016 (from clone | 1.7 | |
| | | DKFZp564P016) | | |
| 343 | Z60848 | H. sapiens CpG island DNA genomic Mse1 fragment, clone | 1.7 | |
| | | 36g10, forward read cpg36g10.ft1a | | |
| 344 | Z28054 | S. cerevisiae chromosome XI reading frame ORF YKL054c | 1.7 | |
| 345 | S79213 | phosphatase inhibitor-2 = cytosolic regulatory subunit of type | 1.7 | |
| | | 1 protein phosphatase [rats, brain, mRNA, 867 nt] | | |
| 346 | X82265 | C. anuum mRNA for 1-aminocylopropane-1-carboxylate | 1.6 | |
| 347 | U46561 | Tetrahymena thermophila polyubiquitin (TTU3) gene, | 1.5 | |
| | | complete cds, and RNA polyinerase II subunit 2 | | |
| 348 | M12132 | Quail fast skeletal muscle troposin I gene, complete cds. | 1.5 | |
| 349 | X98097 | M. musculus promoter region | 1.4 | |
| 350 | D29963 | Homo sapiens mRNA for CD151, complete cds | 1.4 | |
| 351 | D10471 | Herpes simplex virus type 2 genomic DNA for 0.74-0.84 | 1.4 | |
| | | region, complete cds | | |
| 352 | U34673 | Micoureus demerarae cytochrome b light strand gene, | 1.3 | |
| | | mitochondrial gene encoding niltochondrial protein, | | |
| 353 | M15274 | Human Pro-tRNA and Leu-tRNA genes. | 1.3 | |
| 354 | AJ000486 | Trichomonas vaginalis mgl1 gene | 1.3 | |
| 355 | Z47075 | Caenorhabditis elegans cosmid E02H1, complete sequence | 1.3 | |
| | | [Caenorhabditis elegans] | | |
| 356 | NM_001854.1 | Homo sapiens collagen, type XI, alpha 1 type XI collagen | 1.3 | |
| | | (COL11A1)mRNA, complete cds. | | |
| 357 | U37056 | Clostridium cellulovoran endo-1,4-beta glucanase EngF | 1.3 | |
| | | (engF) gene, complete cds | | |
| 358 | U53328 | Human cyclin G mRNA, complete cds. | 1.3 | |
| 359 | Z48230 | Caenorhabditis elegans cosmid F42G10, complete sequence | 1.3 | |
| | | [Caenorhabditis elegans] | | |
| 360 | L42102 | Homo sapiens (subclone 2_c7 from P1 H25) DNA sequence. | 1.3 | |
| 361 | X89417 | S. cerevisiae DNA for protein phosphatase T gene | 1.3 | |
| 362 | Z79884 | H. sapiens chromosome 22 CpG island DNA genomic Mse1 | 1.3 | |
| | | fragment, clone 303c5, complete read | | |
| 363 | 768129 | H. sapiens IDH gamma gene and TRAP delta gene | 1.3 | |
| 364 | U13800 | Human insulin-like growth factor I (IGF1) gene, intron 4. | 1.3 | |
| 365 | L28995 | Oryza saliva 3-hydroxy-3-methylglutaryl coenzyme A | 1.3 | |
| | | reductase gene, complete cds. | | |
| 366 | L43493 | Saccharomyces cerevisiae Jsn1 gene, complete cds | 1.3 | |
| 367 | X08066 | Caenorhabditis elegans myo-2 gene for myosin heavy chain | 1.3 | |
| | | 2 (MHC-C) | | |
| 368 | U37056 | Clostridium Cellulovorane endo-1,4-beta glucanase EngF | 1.3 | |
| | | (engF) gene, complete cds | | |
| 369 | U19905 | Rickettsia tsutsugamushi TA716 56 kDa type-specific | 1.3 | |
| | | antigen gene, complete cds. | | |
| 370 | U74496 | Human chromosome 4q35 subtelomeric sequence | 1.3 | |
| 371 | U46781 | Pasteurella haemolytica putative coproporphyrinogen III | 1.3 | |
| | | oxidase (hemN′) gene, partial cds, leukotoxin transcriptional | | |
| | | and restriction modification methylase subunit | | |
| 372 | X77300 | S. scrofa genomic DNA microsatellite SO344 | 1.3 | |
| 373 | U53745 | Feline immunodeficiency virus isolate FIV-Pco336-7 pol | 1.3 | |
| | | polyprotein (pol) gene, partial cds | | |
| 374 | L08266 | Mouse Facc mRNA, complete cds. | 1.3 | |
| 375 | X52519 | Human gene for tyrosine aminotransferase (TAT) | 1.3 | |
| 376 | U87543 | Aedes aegypti steroid honnone receptor homolog protein | 1.3 | |
| | | gene, complete cds | | |
| 377 | D16682 | Mycoplasma hyopneumoniae gene for 46 kDa surface | 1.3 | |
| | | antigen, complete cds | | |
| 378 | AF022725 | Hordeum vulgare limit dextrinase (HvLD99) gene, complete | 1.3 | |
| 379 | L18987 | Human alpha-2 type XI collagen mRNA, partial cds. | 1.3 | |
| 380 | Y09400 | S. scrofa mRNA for apical organic cation transporter protein | 1.3 | |
| 381 | U10117 | Human endothelial-monocyte activating polypeptide II | 1.3 | |
| | | mRNA, complete cds. | | |
| 382 | U02618 | Saccharomyces cerevisiae molasses resistance | 1.3 | |
| 383 | Z60848 | H. sapiens CpG island DNA genomic Mse1 fragment, clone | 1.3 | |
| | | 36g10, forward read cpg36g10.ft1a | | |
| 384 | X63203 | H. sapiens gene for pregnancy specific beta-1 glycoprotein > | 1.3 | |
| | | :: gb\|S49771\|S49771 pregnancy-specific beta 1 glycoprotein | | |
| | | {5′ region, promoter} [human,placenta, Genomic, 3036 nt] | | |
| 385 | L31854 | Mus musculus Ig epsilon-chain C gene, exon 4, Mg | 1.3 | |
| 386 | X16588 | B. nigra repeat DNA (clone pBN 35) | 1.3 | |
| 387 | U25342 | Xenopus laevis epithelial sodium channel, gamma subunit | 1.3 | |
| | | (gammaxENaC) mRNA, complete cds. | | |
| 388 | L40806 | Neurospora crassa open reading frame gene, complete cds, | 1.3 | |
| | | met-10+ gene, complete cds | | |
| 389 | S49760 | diacylglycerol kinase [rats, brain, mRNA, 3043 nt] | 1.3 | |
| 390 | L15328 | Saccharomyces cerevisiae RNA helicase gene, complete cds. | 1.3 | |
| 391 | S50809 | protein LG = immunoglobulin binding protein Recombinant | 1.3 | |
| 392 | U14662 | Baboon herpesvirus HVP2 gB glycoprotein (UL27) gene, | 1.3 | |
| | | complete cds. | | |
| 393 | U13173 | Human intestinal H+/peptide cotransporter (Hpept1) gene | 1.3 | |
| | | complete cds | | |
| 394 | W71212 | me33e04.r1 Sores mouse embryo NbME13.5 14.5 Mus | 1.3 | |
| | | musculus cDNA clone 389310 5′ similar to | | |
| | | SW:S105_HUMAN P33763 S100 CALCIUM-BINDING | | |
| | | PROTEIN A5;. > :: emb\|X99921\|MMS100A13 M. musculus | | |
| 395 | U25536 | Petunia hybrida alcohol dehydrogenase-2 (Adh2) gene, | 1.3 | |
| | | promoter, 5′UTR, and partial cds. | | |
| 396 | X75014 | M. musculus Phox2 mRNA for homeodomain protein | 1.3 | |
| 397 | Z49436 | S. cerevisiae chromosome X reading frame ORF YJL161w | 1.3 | |
| 398 | X12780 | Chicken MHC class I (B-F) mRNA F10 | 1.3 | |
| 399 | X04319 | E. coli fhuB gene involved in transport of ferrichrome | 1.3 | |
| 400 | U61297 | Human progesterone receptor (PGR) gene, far 5′flanking | 1.3 | |
| 401 | X99518 | Herpesvirus saimiri virion, transformation-associated region, | 1.3 | |
| | | strain C139 | | |
| 402 | M24001 | Mink enteritis virus antigenic type 2 capsid protein genes | 1.3 | |
| | | VP1 and VP2, complete cds. | | |
| 403 | U44877 | Arabidopsis thaliana geranylgeranyl pyrophosphate synthase | 1.3 | |
| | | (GGPS3) mRNA, partial cds | | |
| 404 | AB003431 | Incilaria fruhstorferi mRNA for Incilarin B, complete cds | 1.3 | |
| 405 | M12792 | Human steroid 21-hydroxylase (P-450(C21)) B gene, | 1.3 | |
| | | complete cds, clone lambda-C21B-1. | | |
| 406 | M28548 | Human mutant 21-hydroxylase B gene, complete cds. | 1.3 | |
| 407 | Y10260 | H. sapiens EYA1A gene | 1.3 | |
| 408 | U41740 | Human trans-Golgi p230 mRNA, complete cds | 1.3 | |
| 409 | X02295 | Paramecium 5′ region of gene for surface protein 51A | 1.3 | |
| 410 | S45791 | band 3-related protein = renal anion exchanger AE2 homolog | 1.3 | |
| | | [rabbits, New Zealand White, ileal epithelial cells, mRNA, | | |
| 411 | AE000625. | Helicobacter pylori 26695 section 103 of 134 of the | 1.3 | |
| 412 | X16028 | R. norvegicus gene encoding alkaline phosphatase, exon 3 | 1.3 | |
| | | and joined CDS | | |
| 413 | M73461 | Saccharomyces cerevisiae FL100 RNA14 gene, complete | 1.3 | |
| 414 | L08845 | Drosophila melanogaster disabled mRNA,complete cds | 1.3 | |
| 415 | AE000635.1 | Helicobacter pylori 26695 section 113 of 134 of the | 1.2 | |
| 416 | L39962 | Medicago sativa middle repetitive DNA | 1.2 | |
| 417 | U55371 | Caenorhabditis elegans cosmid T19F4. | 1.2 | |
| 418 | X13679 | Oryza sativa H3 histone pseudogene H3R-12 | 1.2 | |
| 419 | J00223 | Homo sapiens epsilon-1 pseudogene (IGHEP1) gene, CH3 | 1.2 | |
| | | and CH4 regions, exons 3 and 4 and partial sequence | | |
| 420 | AE000649.1 | Helicobacter pylori 26695 section 127 of 134 of the | 1.2 | |
| 421 | X52256 | A. thaliana tufA gene for elongation factor Tu | 1.2 | |
| 422 | M81388 | Chilo iridescent virus DNA-directed RNA polymerase and | 1.2 | |
| | | helicase genes, complete cds's. > :: gb\|S75674\|S75674 DNA | | |
| | | helicase homolog, DNA-depenent RNA polymerase largest | | |
| | | subunit homolog {fragment M} [Chilo iridescent virus CIV, | | |
| 423 | S57565 | histamine H2-receptor [rats, Genomic, 1928 nt] | 1.2 | |
| 424 | X84347 | H. sapiens mRNA for sperm adhesion molecule hPH-20 | 1.2 | |
| 425 | X53579 | A Ahaliana agamous (AG) gene | 1.2 | |
| 426 | Y09539 | L. japonicus gene encoding RING finger protein | 1.2 | |
| 427 | L05500 | Human fetal brain adenylyl cyclase mRNA, 3′ end. | 1.2 | |
| 428 | X68019 | Feline Immunodeficiency Virus GAG gene | 1.2 | |
| 429 | L76739 | Human immunodeficiency virus type 2 (HIV-2 ARM) | 1.2 | |
| | | proviral surface glycoprotein (gp125) gene, partial cds Type | | |
| | | 2 partial envelope sequence, isolate arm from mother in | | |
| 430 | L38769 | Pisolithus tinctorius (F00035) mRNA, EST0049. | 1.2 | |
| 431 | L39786 | Lupinus angustifolius conglutin gamma gene, complete cds | 1.2 | |
| 432 | D10510 | Homo sapiens MAT gene for mitochondrial acetoacetyl- | 1.2 | |
| | | CoA thiolase, exon 11 | | |
| 433 | U47687 | Streptococcus pneumoniae immunoglobulin A1 protease | 1.2 | |
| | | (iga) gene, complete cds | | |
| 434 | U63922 | Xenopus laevis beta-transducin repeat containing protein-3 | 1.2 | |
| | | mRNA, partial cds | | |
| 435 | Z27234 | S. tuberosum STACS2 gene for 1-Aminocyclopropane-1- | 1.2 | |
| | | carboxylate synthase | | |
| 436 | X65365.1 | R. norvegicus GHF1 gene, exon 2B and alternative spliced | 1.2 | |
| 437 | X04336 | Podospora anserina race A mitochondrial DNA class II | 1.2 | |
| | | intron downstream of alpha-sen DNA near CoI gene 5′end | | |
| 438 | X12864 | Yeast (S. douglassi) NAM2 gene for mitochondrial leucyl- | 1.2 | |
| | | tRNA synthetase (EC 6.1.1.4) | | |
| 439 | U66032 | Methanosarcina thermophila CO dehydrogenase/acetyl-CoA | 1.2 | |
| | | synthase alpha subunit (cdhA), epsiolon subunit (cdhB), beta | | |
| | | subunit (cdhC), and NifH class IV protein homolog genes, | | |
| | | complete cds, CO dehydrogenase/acetyl-CoA synthas . . . | | |
| 440 | L08266 | Mouse Facc mRNA, complete cds. | 1.2 | |
| 441 | M64085 | Mouse spi2 proteinase inhibitor (spi2/eb1) mRNA, 3′ end. | 1.2 | |
| 442 | X12773 | Strongylocentrotus purpuratus Spec2d gene 5′-flank and | 1.2 | |
| 443 | U13988 | Peanut chlorotic streak caulimovirus, complete genome. | 1.2 | |
| 444 | U23180 | Caenorhabditis elegans cosmid C28F5 | 1.2 | |
| 445 | M20537 | Mouse thyrotropin beta-subunit gene, exon 5. | 1.2 | |
| 446 | U25881 | Agrius cingulata NADH dehydrogenase subunit 1 protein, | 1.2 | |
| 447 | Y08581 | F. rubripes hsp70-4 gene, complete | 1.2 | |
| 448 | L31848 | Homo sapiens serine/threonine kinase receptor 2 | 1.2 | |
| 449 | M15840 | Human interleukin 1-beta (IL1B) gene, complete cds. | 1.2 | |
| 450 | Z23977 | H. sapiens (D6S443) DNA segment containing (CA) repeat; | 1.2 | |
| | | clone AFM277wb5; single read | | |
| 451 | X14592 | P. hybrida chsB gene for chalcone synthase | 1.2 | |
| 452 | Z49900 | P. sativum mRNA for small GTP-binding protein | 1.2 | |
| 453 | U28154 | Haemophilus somnus cryptic propliage genes, capsid | 1.2 | |
| | | scaffolding protein gene, partial cds, major capsid protein | | |
| | | precursor, endonuclease, capsid completion protein, tail | | |
| | | synthesis proteins, holin, and lysozyme genes, complet . . . | | |
| 454 | D13987 | Brassica napus PE3-PEPCase gene phosphoenolpyruvate | 1.2 | |
| | | carboxylase, complete cds | | |
| 455 | D45243 | Mouse mRNA for ctk, complete cds | 1.2 | |
| 456 | U81144 | Caenorhabditis elegans non-alpha nicotinic acetylcholine | 1.2 | |
| | | receptor subunit precursor (unc-29) gene, complete cds | | |
| 457 | Z92970 | Caenorhabditis elegans cosmid H06O01, complete sequence | 1.2 | |
| | | [Caenorhabditis elegans] | | |
| 458 | L44118 | Homo sapiens proximal CMT1A-REP repeat | 1.2 | |
| 459 | M17120 | D. melanogaster achaete encoding differentiation, | 1.2 | |
| | | complete cds. | | |
| 460 | U55737 | Human ataxia-telangiectasia (ATM) exon42 | 1.2 | |
| 461 | X63525 | P. vulgaris loxA gene for lipoxygenase | 1.2 | |
| 462 | D16402 | Fruitfly Dcdrk gene for Dcdrk kinase, complete cds | 1.2 | |
| 463 | M65287 | Mouse activin receptor (ActR) mRNA, complete cds. | 1.2 | |
| 464 | X13679 | Oryza sativa H3 histonepseudogene H3R-12 | 1.2 | |
| 465 | D28484 | Aspergillus oryzae pgkA gene for phosphoglycerate kinase | 1.2 | |
| | | cds > :: dbj\|E04898\|E04898 gDNA encoding | | |
| 466 | AE000283 | Escherichia coli K-12 MG1655 section 173 of 400 of the | 1.2 | |
| | | complete genome | | |
| 467 | X99832 | H. sapiens CLN3 gene, complete CDS | 1.2 | |
| 468 | U06864 | Rattus norvegicus follistatin-related protein | 1.2 | |
| | | mRNA, complete cds. | | |
| 469 | M87710 | Human simple repeat polymorphism. | 1.2 | |
| 470 | M64497 | Human apolipoprotein AI regulatory protein (ARP-1) | 1.2 | |
| | | mRNA, complete cds. | | |
| 471 | M87710 | Human simple repeat polymorphism. | 1.2 | |
| 472 | X60196 | D. melanogaster partial Mhc gene for myosin heavy chain | 1.2 | |
| 473 | X99719 | S. enterica hsdM, hsdS & hsdR genes | 1.2 | |
| 474 | Z95706 | Microtus rossiaemeridionalis repetitive DNA | 1.2 | |
| 475 | L76372 | Musca domestica (clone F0) arylphorin mRNA fragment | 1.2 | |
| 476 | D26359 | Exogenous mouse mammary tumor virus gene for | 1.2 | |
| | | superantigen, complete cds | | |
| 477 | NM_000694.1 | Homo sapiens aldehyde dehydrogenase 7 (ALDH7) mRNA > | 1.2 | |
| | | :: gb\|U10868\|HSU10868 Human aldehyde dehydrogenase | | |
| | | ALDH7 mRNA, complete cds. | | |
| 478 | AF015882 | Caenorhabditis elegans protein tyrosine phosphatase (ptp-2) | 1.2 | |
| | | mRNA, complete cds | | |
| 479 | M58047 | Mouse 2′,3′-cyclic-nucleotide 3′-phosphodiesterase gene, | 1.2 | |
| 480 | U71121 | Arabidopsis pyruvate decarboxylase-1 (Pdc1) gene, | 1.2 | |
| 481 | U60804 | Danio rerio tumor suppressor p53 (p53) mRNA, complete | 1.2 | |
| 482 | X99254 | P. falciparum gene encoding primase, small subunit | 1.2 | |
| 483 | D10197 | Bovine mRNA for histamine H1 receptor, complete cds | 1.2 | |
| 484 | Y09764 | Homo sapiens GABRE gene, exon 2-8 | 1.2 | |
| 485 | U72396 | Lycopersicon esculentum class II small heat shock protein | 1.2 | |
| | | Le-HSP17.6 mRNA, complete cds | | |
| 486 | X72950 | X. laevis H31 gene for histone H3 | 1.2 | |
| 487 | D29956 | Human mRNA for KIAA0055 gene, complete cds | 1.2 | |
| 488 | X56003 | E. coli (plasmidpFM205) faeE and faeF genes | 1.2 | |
| 489 | M64269 | Human mast cell chymase gene, complete cds. | 1.2 | |
| 490 | AB002963 | Human immunodeficiency virus type 1 env gene for | 1.2 | |
| | | envelope glycoprotein, partial cds, clone 205E5B2t | | |
| 491 | X90846 | H. sapiens mRNA for mixed lineage kinase 2 | 1.2 | |
| 492 | X03715 | Spiroplasma melliferum tRNA gene cluster | 1.2 | |
| 493 | U83494 | Tropidurus hispidus ATPase subunit 6 (ATPase6) gene, | 1.2 | |
| | | mitochondrial gene encoding mitochondrial protein, partial | | |
| 494 | U60804 | Danio rerio tumor suppressor p53 (Ps3) mRNA complete | 1.2 | |
| 495 | M24081 | Tetrahymena pyriformis (clone pTU2) ubiquitin genes, 3′and | 1.2 | |
| 496 | U54803 | Mus musculus cysteine protease (Lice) gene, exons 3-7, and | 1.2 | |
| | | complete cds | | |
| 497 | L13748 | Human dihydrolipoamide dehydrogenase gene, exon 1. | 1.2 | |
| 498 | U29390 | Chrysosporium parvum 18S ribosomal RNA gene, partial | 1.2 | |
| 499 | L81694 | Homo sapiens (subclone 1_d1 from P1 H56) DNA sequence | 1.2 | |
| 500 | X65366.1 | R. norvegicus GHF1 gene, intron 2 | 1.2 | |
| 501 | U74651 | Human DNA polymerase gamma (polg) gene, promoter | 1.2 | |
| | | region and partial cds | | |
| 502 | X65591 | S. mansoni mRNA for myosin II heavy chain | 1.2 | |
| 503 | D17255 | Human HepG2 3′ region MboI cDNA, clone hmdsc12m3 | 1.2 | |
| 504 | L33792 | Senecio odorus lipid transfer protein mRNA, 3′ end. | 1.2 | |
| 505 | L13612 | Drosophila melanogaster dead-box protein (dbp45A) gene, | 1.2 | |
| | | complete cds. > :: emb\|Z23266\|DMDEADBXA | | |
| | | D. melanogaster DEAD-box gene, complete CDS | | |
| 506 | X81650 | M. musculus for c-ros protooncogene | 1.2 | |
| 507 | NM_000037.1 | Homo sapiens ankyrin 1, erythrocytic (ANK1) mRNA > :: | 1.2 | |
| | | gb\|M28880\|HUMANK Human erythroid ankyrin mRNA, | | |
| 508 | X98543 | A. thaliana endo-1,4-beta-glucanase gene | 1.2 | |
| 509 | D89501 | Human PBI gene, complete cds | 1.2 | |
| 510 | Z82174 | Human DNA sequence from cosmid B20F6 on chromosome | 1.2 | |
| | | 22, complete sequence [Homo sapiens] | | |
| 511 | M36881 | Human lymphocyte-specific protein tyrosine kinase | 1.2 | |
| 512 | U92014 | Human clone 121711 defecfive mariner transposon Hsmar2 | 1.2 | |
| | | mRNA sequence | | |
| 513 | U09948 | Morone saxatilis Hox-B5-like homeodomain protein gene, | 1.2 | |
| 514 | M58155 | African swine fever virus multigene families 360 and 110. | 1.2 | |
| 515 | U30500 | Sicilian sandfly fever virus glycoprotein precursor | 1.2 | |
| | | polypeptide mRNA, complete cds. | | |
| 516 | L09190 | Human trichohyalin (TRHY) gene, complete cds. | 1.2 | |
| 517 | U67508 | Methanococcus jannaschii section 50 of 150 of the complete | 1.2 | |
| 518 | M81186 | Clostridium botulinum neurotoxin type B (botB) gene | 1.2 | |
| 519 | U93037 | Homo sapiens elastin gene, exons 5-27 and alternatively | 1.2 | |
| | | spliced products, partial cds | | |
| 520 | D87454 | Human mRNA for KIAA0265 gene, partial cds | 1.2 | |
| 521 | D87558 | Gallus gallus mRNA for C-Serrate-2, partial cds | 1.2 | |
| 522 | Z32847 | L. infantum (10541) kmetoplast DNA | 1.2 | |
| 523 | U09584 | Human PL6 protein (PL6) mRNA, complete cds. | 1.2 | |
| 524 | AC001530 | Homo sapiens (subclone 2_b8 from P1 H56) DNA sequence | 1.2 | |
| 525 | X74322 | H. sapiens gap-I gene | 1.2 | |
| 526 | D29792 | Mouse gene for T cell receptor gamna chain | 1.2 | |
| 527 | M24001 | Mink enteritis virus antigenic type 2 capsid protein genes | 1.2 | |
| | | VP1 and VP2, complete cds. | | |
| 528 | K02819 | Rabbit MHC RLA region class I 19-1 gene, complete cds. | 1.2 | |
| 529 | L33879 | Insertion sequence IS1245 (from Mycobacterium avium) | 1.2 | |
| | | transposase gene, complete cds. | | |
| 530 | AE000607.1 | Helicobacter pylori 26695 section 85 of 134 of the complete | 1.2 | |
| 531 | W71212 | me33e04.r1 Soares embryo NbME13.5 14.5 Mus | 1.2 | |
| | | musculus cDNA clone 389310 5′ similar to | | |
| | | SW:S105_HUMAN P33763 S100 CALCIUM-BINDING | | |
| | | PROTEIN A5;. > :: emb\|X99921\|MMS100A13 M. musculus | | |
| 532 | X83078 | M. musculus nid gene (exon 4) | 1.2 | |
| 533 | U95041 | Rattus norvegicus transcriptional corepressor KAP1/TIF1B | 1.2 | |
| | | mRNA, partial cds | | |
| 534 | X58907 | H. sapiens CYP21 gene for steroid 21-monooxygenas | 1.2 | |
| 535 | L11669 | Human tetracycline transporter-like protein mRNA, | 1.2 | |
| 536 | L37053 | Gorilla gorilla (clone Gor-ID) Rhesus-like protein mRNA, | 1.2 | |
| 537 | M33782 | Human TFEB protein mRNA, partial cds. | 1.2 | |
| 538 | D78172 | Spinacia oleracea mRNA for 26S proteasome beta subunit, | 1.2 | |
| | | complete cds | | |
| 539 | M33782 | Human TFEB protein mRNA, partial cds. | 1.2 | |
| 540 | M33782 | Human TFEB protein mRNA, partial cds. | 1.2 | |
| 541 | Z54312 | L. sake las ]A, M, P, T] genes | 1.1 | |
| 542 | S81773 | inwardly rectifying K+ channel IRK3(HIT) [hamsters, | 1.1 | |
| | | insulinoma cell line HIT-T15, mRNA Partial, 1791 nt] | | |
| 543 | Z78910 | H. sapiens flow-sorted chromosome 6 HindIII fragment, | 1.1 | |
| 544 | U08408 | Xenopus laevis arginase 3 inRNA, complete cds. | 1.1 | |
| 545 | U26444 | Bacillus subtilis 2,3-dihydro-2,3-dihydroxybenzoate | 1.1 | |
| | | dehydrogenase (dhbA), isochorismate synthase (dhbC), 2,3- | | |
| | | dihydroxybenzoate-AMP ligase (dhbE), and isochorismatase | | |
| | | (dhbB) genes, complete cds, and (dhbF) gene, partial cds | | |
| 546 | X62170 | N. crassa bli-7 gene | 1.1 | |
| 547 | U94403 | Rattus norvegicus proton gated cation channel ASIC1 | 1.1 | |
| | | mRNA, complete cds | | |
| 548 | AJ000498 | Homo sapiens DNA for integration site of HBV in a | 1.1 | |
| | | hepatocellular carcinoma | | |
| 549 | X99485 | L. luteus mRNA for alpha-subunit of G protein | 1.1 | |
| 550 | U67520 | Methanococcus jannaschii section 62 of 150 of the complete | 1.1 | |
| 551 | L25415 | Mycoplasma pulmonis glutamyl tRNA synthetase (gltX) | 1.1 | |
| | | gene restriction-modification enzyme subunits S1A, R1, | | |
| | | M1, S1B (hsds1A, hsdR1, hsdM1, hsds1B) genes, complete | | |
| | | cds's, DNA polymerase III (polC) gene, 3' region. | | |
| 552 | X68107 | M. sativamsCHSII mRNA for chalcone synthase | 1.1 | |
| 553 | X89246 | D. melanogaster mRNA for DHR38 protein | 1.1 | |
| 554 | X64332 | C. lewisii PgiC2-a gene for phosphoglucose isomerase | 1.1 | |
| 555 | U47331 | Rattus norvegicus metabotropic glutamate receptor 4b | 1.1 | |
| | | mRNA, complete cds. | | |
| 556 | X16055 | Bacteriophage T4 gene 20 encoding gp20, structural protein | 1.1 | |
| 557 | D00596 | Homo sapiens gene for thyrmdylate synthase, exons 1, 2, 3, | 1.1 | |
| | | 4, 5, 6, 7, complete cds | | |
| 558 | X14639 | Tomato ribosomal DNA intergenic spacer | 1.1 | |
| 559 | U67520 | Methanococcus jannaschii section 62 of 150 of the complete | 1.1 | |
| 560 | Y11786 | R. prowazekii ksgA gene and 2 open reading frames | 1.1 | |
| 561 | Z81065 | Caenorhabditis elegans cosmid F16C3, complete sequence | 1.1 | |
| | | [Caenorhabditis elegans] | | |
| 562 | X60694 | C. perfringens plasmid epsilon-toxin gene | 1.1 | |
| 563 | X52648 | Schizosaccharomyces pombe p68 gene for p68 protein | 1.1 | |
| 564 | X04078 | Potato patatin pseudogene (SA10C) | 1.1 | |
| 565 | U38783 | Schizosaccharomyces pombe brefeldin A resistance protein | 1.1 | |
| | | (hba1) and unknown orf genes, complete cds | | |
| 566 | U32769 | Haemophilus influenzae Rd section 84 of 163 of the | 1.1 | |
| 567 | D89066 | Staphylococcus aureus DNA for DnaA, complete cds | 1.1 | |
| 568 | U07797 | Rattus norvegicus Sprague-Dawley (T1-alpha) mRNA, | 1.1 | |
| 569 | L14710 | C. elegans cosmid K02D10. | 1.1 | |
| 570 | U42599 | Borrelia burgdorferi plasmid cp18, OspE (ospE) gene, | 1.1 | |
| 571 | U48726 | Human epidermal growth factor receptor (EGFR) precursor- | 1.1 | |
| | | mRNA, exons 8 and 9, partial cds | | |
| 572 | U38844 | Xenopus laevis cyclin-dependent kinase inhibitor p28 gene, | 1.1 | |
| | | complete cds | | |
| 573 | S82864 | Elk-3 = Ets transcription factor [mice, 16-day embryos, | 1.1 | |
| 574 | X65720 | M. musculus gene for protein kinase C-gamma (exon1 and | 1.1 | |
| 575 | D14484 | Hepatitis C virus strain J33 genomic RNA, complete genome | 1.1 | |
| 576 | L11998 | Staphylococcus aureus conjugative transfer gene complex | 1.1 | |
| 577 | D14339 | Rice mitochondrion DNA for ATPase subunit 6 and ORFs, | 1.1 | |
| | | complete cds | | |
| 578 | D38413 | Yeast DNA for Ppf2p, complete cds | 1.1 | |
| 579 | D90210 | Bacteriophage c-st (from C. botulinum) C1-tox gene or | 1.1 | |
| | | botulinum C1 neurotoxin | | |
| 580 | X67838 | B. napus DNA for myrosinase | 1.1 | |
| 581 | X17053 | Rat immediate-early serum-responsive JE gene | 1.1 | |
| 582 | X12426 | Xenopus laevis U1 70K gene exon 4, 5, 6 and 7 | 1.1 | |
| 583 | U55043 | Bacillus subtilis plasmid pPOD2000 Rep, RapAB, RapA, | 1.1 | |
| | | ParA, ParB, and ParC genes, complete cds. | | |
| 584 | M34046 | Human placental protein 14 (PP14) gene, complete cds. | 1.1 | |
| 585 | U52367 | Clostridium acetobutylicum ATCC 824 F-type ATP synthase | 1.1 | |
| | | subunit a (atpA) gene, F-type ATP synthase subunit c (atpC) | | |
| | | gene, and F-type ATP synthase subunit b | | |
| 586 | Z35955 | S. cerevisiae chromosome II reading frame ORF YBRO86c | 1.1 | |
| 587 | U53179 | Caenorhabditis elegans cosmid T27B2. | 1.1 | |
| 588 | X77253 | C. herbarum Cla h III mRNA > ::gb\|I26207\|I26207 | 1.1 | |
| | | Sequence 1 from U.S. Pat. No. 5556953 | | |
| 589 | X72713 | A franciscana mRNA for Sarco/endoplasmic reticulum Ca- | 1.1 | |
| 590 | U29145 | Caenorhabditis elegans (mab-18) mRNA, transcript | 1.1 | |
| 591 | U06061 | Cubanichthys pengelleyi mitochondrial control region | 1.1 | |
| | | partial sequence | | |
| 592 | X62244 | B. mycoides blacI gene for beta-lactamase I | 1.1 | |
| 593 | M88355 | Mouse oxytocin-neurophysin I gene, complete cds | 1.1 | |
| 594 | U83489 | Emericella nidulans septin B (aspB) mRNA, complete cds | 1.1 | |
| 595 | M18193 | Human inter-alpha-tiypsin inhibitor heavy chain mRNA, | 1.1 | |
| | | partial cds, clones lambda-HuHITI-[9, 33]. | | |
| 596 | U71273 | Sus scrofa glucosidase II mRNA, complete cds | 1.1 | |
| 597 | S53497 | immunoglobulin epsilon chain constant region = secreted | 1.1 | |
| | | form {3′ region} [human, B cell myeloma U-266, Genomic, | | |
| 598 | U25119 | Caenorhabditis elegans calcium channel alpha-1 subunit | 1.1 | |
| | | homolog Unc-2 (unc-2) gene, partial cds. | | |
| 599 | U56959 | Caenorhabditis elegans cosmid T21F4. | 1.1 | |
| 600 | X99485 | L. luteus mRNA for alpha-subunit of G protein | 1.1 | |
| 601 | L35848 | Homo sapiens IgE receptor beta chain (HTm4) mRNA, | 1.1 | |
| 602 | U93237 | Human menin (MEN1) gene, complete cds | 1.1 | |
| 603 | L07042 | Medicago sativa MAP kinase MsERK1 mRNA, complete | 1.1 | |
| 604 | Z36977 | N. plumbaginifolia mRNA for catalase (cat3 gene) | 1.1 | |
| 605 | J00738 Rattus norvegicus submaxillary gland alpha-2u globulin | 1.1 | | |
| | | mRNA, complete cds. | | |
| 606 | X03018 | Xenopus laevis histone gene cluster X1H3-A with gene | 1.1 | |
| | | H1A, H2B, H3 and H4 | | |
| 607 | X68449 | L. esculentum U6 snRNA pseudogene (LeU6.1ps) | 1.1 | |
| 608 | U53921 | Pneumocystis carinii major surface glycoprotein | 1.1 | |
| 609 | M87106 | Human immunodeficiency virus type 2 (FOPOLC2) | 1.1 | |
| | | polymerase fragment. > :: gb\|M87107\|HIVPOLC3 Human | | |
| | | immunodeficiency virus type 2 (FOPOLC3) polymerase | | |
| 610 | U84539 | Human dystrobrevin (DTN) gene, exon 11A | 11.1 | |
| 611 | J02896 | S. purpuratus speract egg protein mRNA, complete cds. | 1.1 | |
| 612 | J02896 | S. purpuratus speract egg protein mRNA, complete cds. | 1.1 | |
| 613 | AF016253 | Klebsiella aerogenes D-amino acid dehydrogenase | 1.1 | |
| 614 | L22173 | Saccharomyces cerevisiae aminonitrophenyl propanediol | 1.1 | |
| | | (ANP1), UV excision repair protein (RAD23), cytochrome c | | |
| | | isozyme (CYC7) genes, complete cds. | | |
| 615 | U41357 | Tetrahymena thermophila P-type ATPase (TPA11) gene, | 1.1 | |
| 616 | X14383 | Bunyamwera virus L protein RNA, complete cds | 1.1 | |
| 617 | U50378 | Mus musculus DNA repair enzyme (Ku 70) gene, exon 13 | 1.1 | |
| | | and complete cds | | |
| 618 | Z18278 | Mus musculus of 5HT5 receptor cDNA gene encoding | 1.1 | |
| | | 5HT5 serotonin receptor | | |
| 619 | U14172 | Mus musculus p162 protein mRNA, complete cds. | 1.1 | |
| 620 | X76762 | H. sapiens serotonin transporter gene, exon 14 | 1.1 | |
| 621 | U62055 | Bacillus subtilis CzcD (czcD) gene, partial cds, TrkA (trkA) | 1.1 | |
| | | gene, complete cds | | |
| 622 | X81847 | E. carotovora pel1, pel2 and pel3 genes | 1.1 | |
| 623 | M25477 | Caenorhabditis elegans collagen (col6) gene, complete cds. | 1.1 | |
| 624 | X97196 | D. melanogaster X gene | 1.1 | |
| 625 | L39059 | Homo sapiens transcription factor SL1 mRNA, complete | 1.1 | |
| 626 | M21790 | X. laevis complement component 3 (C3) mRNA, 3′ end. | 1.1 | |
| 627 | X95161 | H. sapiens brca2 gene exon 11 > :: emb\|A62786.1\|A62786 | 1.1 | |
| | | Sequence 27 from Patent WO9719110 | | |
| 628 | K02446 | Chicken smooth-muscle alpha-topomyosin gene, complete | 1.1 | |
| 629 | U24171 | Mus musculus p21 (WAF1) gene, partial promoter sequence | 1.1 | |
| 630 | Z26306 | H. sapiens isoform 1 gene for calcium channel, exons | 1.1 | |
| 631 | U23070 | Human putative transmembrane protein (nma) mRNA, | 1.1 | |
| 632 | AE000046 | Mycoplasma pneumoniae section 46 of 63 of the complete | 1.1 | |
| 633 | X17548 | D. melanogaster singed gene, exon 1 | 1.1 | |
| 634 | U54497 | Xenopus laevis integrin alpha 4 mRNA, complete cds. | 1.1 | |
| 635 | J02793 | Mouse R and L1 (3′ end) repetitive elements. | 1.1 | |
| 636 | D10832 | Equine herpesvirus genomic DNA for ‘TREC’ OCT- | 1.1 | |
| 637 | X51766 | L. polyphyllus mRNA for protein S16 | 1.1 | |
| 638 | U60804 | Danio rerio tumor suppressor p53 (p53) mRNA,complete | 1.1 | |
| 639 | Z79068 | H. sapiens flow-sorted_chromosome 6 HindIII fragment, | 1.1 | |
| 640 | Z73585 | S. cerevisiae chromosome XVI reading frame ORF | 1.1 | |
| 641 | U22818 | Cricetulus griseus SRD-2 mutant sterol regulatory element | 1.1 | |
| | | binding protein-2( SREBP-2) mRNA, complete cds. | | |
| 642 | M25710 | Human thyroid peroxidase (TPO) gene, exon 11. | 1.1 | |
| 643 | L12591 | Human alkaline phosphatase gene, 5′ flanking region. | 1.1 | |
| 644 | L25616 | Homo sapiens kinectin mRNA, complete cds | 1.1 | |
| 645 | U32251 | Bos taurus clone 9 immunoglobulin lambda light chain | 1.1 | |
| | | variable region (Vlambda1b) mRNA, partial cds | 1.1 | |
| 646 | M97516 | Mouse alpha-2 adrenergic receptor gene, complete cds. | 1.1 | |
| 647 | AJ000060 | Mus musculus gene encoding iysosomal hayluronidase, | 1.1 | |
| 648 | L07067 | Simian varicella virus tegument protein gene, complete cds; | | |
| | | serine/threonine protein kinase genes, complete cds; | | |
| | | membrane glycoprotein genes, complete cds's. | | |
| 649 | L00619 | Mouse T-cell-specific tyrosine kinase (Itk) mRNA, complete | 1.1 | |
| 650 | M76981 | Glycine max vspA gene, complete cds. | 1.1 | |
| 651 | D87460 | Human mRNA for KIAA0270 gene, partial cds | 1.1 | |
| 652 | D17466 | Streptomyces setonii crtS gene for sigma factor, complete | 1.1 | |
| 653 | D89066 | Staphylococcus aureus DNA for DnaA, complete cds | 1.1 | |
| 654 | X96853 | P. persica mRNA for endo-beta-1,4-glucanase, pcel1 | 1.1 | |
| 655 | NM_002248.1 | Homo sapiens potassium intermediate/small conductance | 1.1 | |
| | | calcium-activated channel, subfamily N, member 1 | | |
| | | (KCNN1) mRNA > :: gb\|U69883\|HSU69883 Human | | |
| | | calcium-activated potassium channel hSK1 (5K) mRNA, | | |
| 656 | Z63493 | H. sapiens CpG island DNA genomic Mse1 fragment, clone | 1.1 | |
| | | 85c1, reverse read cpg85c1.rt1a | | |
| 657 | M21995 | Human coagulation factor XIII a subunit gene, exon 9. | 1.1 | |
| 658 | X60367 | Mouse CRBPI mRNA for cellular retinol binding protein I | 1.1 | |
| 659 | M63224 | Wheat germin 9f-3.8 gene, complete cds. | 1.1 | |
| 660 | M74515 | Mouse GA binding protein (GABP-alpha subunit) mRNA, | 1.1 | |
| | | complete cds. | | |
| 661 | X52949.1 | G. intestinalis DNA for rRNA tandem repeat unit | 1.1 | |
| 662 | D11388 | Rattus norvegicus gene for ribosomal protein S15, exon | 1.1 | |
| | | 3, 4, complete cds | | |
| 663 | U22302 | Human histo blood group ABO glycosyltransferase | 1.1 | |
| 664 | M38132 | S. pombe rad1 gene, complete cds. | 1 | |
| 665 | U07261 | Perissodus microlepis T51a mitochondrion NADH | 1 | |
| | | dehydrogenase subunit 2 gene, complete cds | | |
| 666 | Z75244 | S. cerevisiae chromosome XV reading frame ORF YOR336w | 1 | |
| 667 | D63816 | Mouse DNA for glutamate trans orter S1c1a3, exon 1 | 1 | |
| 668 | U04699 | Euproctus platycephalus mitochondrion 16S rRNA gene, | 1 | |
| | | partial sequence. | | |
| 669 | M19881 | P. falciparum knop protein (KP) gene, complete cds. | 1 | |
| 670 | X64310 | H. sapiens DNA for pu-py sequence on 11p13 | 1 | |
| 671 | X56469 | Mouse HSA-A gene coding for heat stable antigen | 1 | |
| 672 | L76262 | Meloidogyne hapla mitochondrial COII gene, 3′ end of cds; | 1 | |
| | | transfer RNA-His gene; 16S ribosomal RNA gene; ND3 | | |
| | | complete cds; cytochrome b (cytb) gene, 5′ end of cds. | | |
| 673 | X52574 | Mouse mRNA from Mov10 locus | 1 | |
| 674 | Z11711 | H. sapiens gene for alpha-2 macroglobulin, exon 1 | 1 | |
| 675 | M37240 | P. juncea N8 family repetitive sequence DNA. | 1 | |
| 676 | Z72947 | S. cerevisiae chromosome VII reading frame ORF YGR162w | 1 | |
| 677 | X58713 | N. crassa phr gene for photolyase | 1 | |
| 678 | M30502 | Human immunodeficiency virus type 2 (HIV-2), complete | 1 | |
| | | proviral genome. | | |
| 679 | M23082 | Chicken embryo fibroblast tropomyosin mRNA, complete | 1 | |
| 680 | K03203 | Human PRH1 locus salivary proline-rich protein mRNA | 1 | |
| 681 | M86844 | Microtus chrotorrhinus tandem satellite array DNA sequence | 1 | |
| 682 | U55381 | Leishmania tropica P-glycoprotein E gene, complete cds | 1 | |
| 683 | D11388 | Rattus norvegicus gene for ribosomal protein S15, exon 1, 2, | 1 | |
| | | 3, 4, complete cds | | |
| 684 | L43496 | Xenopus laevis ligase I (ligI) mRNA, complete cds | 1 | |
| 685 | X69103 | C. glutamicum csp2 gene | 0.99 | |
| 686 | M32883 | Alfalfa leghemoglobin gene, complete cds. | 0.98 | |
| 687 | M30502 | Human immunodeficiency virus type 2 (HIV-2), complete | 0.98 | |
| | | proviral genome. | | |
| 688 | K02212 | Human alpha-1-antitrypsin gene (S variant), complete cds. | 0.96 | |
| 689 | Y09746 | H. oligactis mRNA for heat shock protein | 0.96 | |
| 690 | D12580 | Group II phytoplasma gene for 16S ribosomal RNA | 0.95 | |
| 691 | L10465 | Haematobia irritans (clone Horn.fly.3.7) mariner transposase | 0.95 | |
| | | pseudogene, partial cds. | | |
| 692 | U22541 | Enterococcus hirae insertion sequence. | 0.95 | |
| 693 | U66261 | Caenorhabditis elegans multidrug resistance related protein 2 | 0.93 | |
| | | (mrp-2) mRNA, complete cds | | |
| 694 | L05517 | Plasmodium chabaudi DNA sequence, exon 1. | 0.91 | |
| 695 | S40532 | NSCL-2 = basic domain helix-loop-helix gene [mice, embryo, | 0.91 | |
| | | mRNA, 2230 nt] | | |
| 696 | L48339 | Pseudomonas aureofaciens phzFABCD genes, complete | 0.65 | |
| | | cds's. > :: gb\|AR008980\|AR008980 Sequence 11 from patent | | |
| 697 | LX77515 | R. rubrum nifJ gene | 0.65 | |
| 698 | X51394 | Xenopus mRNA for APEG protein, containing a highly | 0.65 | |
| | | repetitive amino acid sequence | | |
| 699 | AF097906 | Rana catesbeiana myosin heavy chain (MHC-3) mRNA, | 0.64 | |
| 700 | X64724 | M. musculus NKR-P1 2 gene for natural killer cell receptor, | 0.62 | |
| 701 | NM_001462.1 | Homo sapiens formyl peptide receptor-like 1 (FPRL1) | 0.61 | |
| | | mRNA > :: gb\|M84562\|HUMFPRL1A Human formyl | | |
| | | peptide receptor-like receptor (FPRL1) mRNA, complete | | |
| 702 | AF110966.1 | HIV-1 isolate C-96BW04.10 country Botswana, complete | 0.6 | |
| 703 | Y10743 | S. scrofa mRNA for p120-PI3K protein | 0.6 | |
| 704 | U39669 | Xenopus laevis pyruvate dehydrogenase E1-beta subunit | 0.59 | |
| | | (PdhE1beta-2) mRNA, partial cds | | |
| 705 | AJ004952 | Bos taurus mRNA for fibroblast growth factor receptor type | 0.59 | |
| 706 | AF125454 | Caenorhabditis elegans cosmid Y47G7C | 0.59 | |
| 707 | X59002 | Leukemogenic retrovirus T1223/B env gene | 0.59 | |
| 708 | Z73039 | S. cerevisiae chromosome VII reading frame ORF YGR254w | 0.59 | |
| 709 | Z28236 | S. cerevisiae chromosome XI reading frame ORF YKR011c | 0.58 | |
| 710 | U39669 | Xenopus laevis pyruvate dehydrogenase E1-beta subunit | 0.58 | |
| | | (PdhE1beta-2) mRNA, partial cds | | |
| 711 | Z16651 | H. sapiens (D10S199) DNA segment contaming | 0.58 | |
| 712 | X60163 | Human TSK39.1 telomere junction | 0.58 | |
| 713 | D10465 | Zymomonas mobilis invA gene for intracellular invertase | 0.58 | |
| | | E1, complete cds | | |
| 714 | Z33280 | M. capricolum DNA for CONTIG MC376 | 0.57 | |
| 715 | AF012899 | Sambucus nigra ribosome inactivating protein precursor | 0.57 | |
| | | mRNA, complete cds | | |
| 716 | AB014572 | Homo sapiens mRNA for KIAA0672 protein, complete cds | 0.57 | |
| 717 | NM_002714.1 | Homo sapiens protein phosphatase 1, regulatory subunit 10 | 0.57 | |
| | | (PPP1R10) mRNA > :: emb\|Y13247\|HSFB19 Homo sapiens | | |
| 718 | M10630 | Bacteriophage U3 cleavage site for phage phi-X174 gene A | 0.56 | |
| 719 | Z55474 H. sapiens CpG island DNA genomic Mse1 fragment, clone | 0.56 | | |
| | | 42d3, reverse read cpg42d3.rt1a | | |
| 720 | U96697 | Drosophila melanogaster DPP2C1 (dpp2c1) mRNA, | 0.56 | |
| 721 | AC001048 | Homo sapiens (subclone 1_f12 from P1 H55) DNA | 0.56 | |
| 722 | M32245 | Human aromatase gene, 5′ flank. | 0.55 | |
| 723 | AF051944 | Gallus gallus Xin mRNA, complete cds | 0.55 | |
| 724 | AF077539 | Caenorhabditis elegans cosmid T25D3 | 0.54 | |
| 725 | U43841 | Entamoeba histolytica U6 small nuclear RNA gene, | 0.54 | |
| | | complete sequence | | |
| 726 | NM_000551.1 | Homo sapiens von Hippel-Lindau syndrome (VHL) mRNA, | 0.54 | |
| | | and translated products | | |
| 727 | U55215 | Cavia porcellus interleukin-5 receptor alpha precursor (gpIL- | 0.53 | |
| | | 5ra) mRNA, complete cds | | |
| 728 | D16471 | Human mRNA, Xq terminal portion | 0.53 | |
| 729 | X76245 | S. cerevisiae NOP77 gene for essential nucleolar protein | 0.52 | |
| 730 | Z11993 | V. proteolyticus aminopeptidase | 0.51 | |
| 731 | L34620 | Eubacterium fosser 16S ribosomal RNA. | 0.49 | |
| 732 | U28757 | Sus scrofa lysozyme gene, complete cds | 0.49 | |
| 733 | U75187 | Arabidopsis thaliana germin-like protein (GLP1) mRNA, | 0.49 | |
| 734 | Z96514 | H. sapiens telomeric DNA sequence, clone 2PTEL005, read | 0.48 | |
| | | 2PTELOO005.seq | | |
| 735 | AE000579.1 | Helicobacter pylori 26695 section 57 of 134 of the complete | 0.48 | |
| 736 | Y13852 | Drosophila diplacantha clone D3 inactive Ban-1 family | 0.47 | |
| 737 | X14036 | Tomato cab-7 gene for type II chlorophyll a/b-binding | 0.47 | |
| 738 | X84308 | H. vulgare mRNA for photosysteme I antenna protein | 0.47 | |
| 739 | S78378 | Oct-4 (t12 haplotype) [mice, Genomic, 1191 nt, segment | 0.46 | |
| 740 | NM_000254.1 | Homo sapiens 5-methyltetrahydrofolate-homocysteine | 0.46 | |
| | | methyltransferase synthase mRNA, complete cds | | |
| 741 | L04161 | Plasmodium falciparum (clone Pfg377 [PfsXLX]) DNA | 0.46 | |
| | | sequence, complete cds | | |
| 742 | X95887 | H. sapiens PLP gene, intron 1 | 0.45 | |
| 743 | U38179 | Rattus norvegicus cyclic nucleotide phosphodiesterase | 0.45 | |
| | | (RNPDE3A) mRNA, complete cds. | | |
| 744 | U41833 | Macaca mulatta MHC class I antigen Mamu B*02 mRNA, | 0.45 | |
| | | complete cds | | |
| 745 | L01794 | Plasmid pAD1 (from Enterococcus faecalis) replication- | 0.45 | |
| | | associate protein (repA, repB, and repC) genes, complete | | |
| 746 | U64573 | Human connexin43 gap junction protein (connexin43) gene, | 0.44 | |
| | | exon 1 and promoter region | | |
| 747 | X60832 | P. sativum gene (GS 3A) for glutamine synthase | 0.44 | |
| 748 | Z93997 | Unidentified bacterium DNA for 16S ribosomal RNA | 0.44 | |
| 749 | U32818 | Haemophilus influenzae Rd section 133 of 163 of the | 0.44 | |
| 750 | AF018161 | Sphaerozoum punctatum 16S-like ribosomal RNA gene, | 0.44 | |
| | | complete sequence | | |
| 751 | D78156 | Human mRNA for rasGTPase activating protein, partial cds | 0.44 | |
| 752 | AB000173 | Porcine mRNA for endopeptidase 24.16, complete cds | 0.44 | |
| 753 | M36626 | Rat simple sequence DNA, clone 5. | 0.44 | |
| 754 | Y09922 | M. musculus flanking region of exon 1 of SEZ-6 gene | 0.44 | |
| | | including promoter sequence | | |
| 755 | X13602 | Caldocellum saccharolyticum celB gene for | 0.44 | |
| | | cellobiohydrolase/endocellulase | | |
| 756 | AF005664 | Homo sapiens properdin (PFC) gene, complete cds | 0.44 | |
| 757 | M63312 | Chinese hamster cAMP-dependent protein kinase, catalytic | 0.44 | |
| | | subunit-beta mRNA, complete cds. | | |
| 758 | U43382 | Human Down Syndrome region of chromosome 21 DNA. | 0.44 | |
| 759 | U75187 | Arabidopsis thaliana germin-like protein (GLP1) mRNA, | 0.44 | |
| 760 | U21914 | Human duplicate spinal muscular atrophy mRNA, clone | 0.43 | |
| | | 5G7, partial cds. | | |
| 761 | AF010537 | Plasmodium falciparum microsatellite TA3 sequence | 0.43 | |
| 762 | X05034 | Rat C2A gene for prostatic binding protein (PBP) | 0.43 | |
| 763 | D16579 | Dictyostelium discoideum mitochondrial DNA for NADH | 0.43 | |
| | | dehydrogenase subunits and cytochrome oxidase subunit | | |
| 764 | M58155 | African swine fever virus multigene families 360 and 110. | 0.43 | |
| 765 | Z82587 | R. prowazekii genomic DNA fragment (clone A315R) | 0.43 | |
| 766 | X13011 | Bacillus subtilis DNA for glyceraldehyde-3-phosphate | 0.43 | |
| | | dehydrogenase (EC 1.2.1.12) | | |
| 767 | X59952 | T. thermophila SB2040 micronuclear limited DNA element | 0.43 | |
| 768 | Z70730 | L. lactis gene for beta-phosphoglucomutase | 0.43 | |
| 769 | X94445 | S. pombe cwl1 gene | 0.43 | |
| 770 | X63628 | S. pombe MFm2 gene | 0.43 | |
| 771 | X60049 | O. berteriana mitochondrial nad5 gene for NADH | 0.43 | |
| | | dehydrogenase subunit 5, exons d and e | 0.43 | |
| 772 | D45241 | Rat MHC class II gene (RTI.DOa), exon 2, 3, 4 and 5 | 0.43 | |
| 773 | D83472.1 | Bos taurus gene for adrenodoxin reductase, exon 1, 2 | 0.43 | |
| 774 | M34044 | Pig Na+/glucose cotransporter protein (SGLT1) mRNA, 3′ | 0.43 | |
| 775 | U28488 | Human putative G protein-coupled receptor (AZ3B) mRNA, | 0.43 | |
| | | complete cds | | |
| 776 | X89398 | H. sapiens ung gene for uracil DNA-glycosylase | 0.43 | |
| 777 | Z36879 | F. pringlei gene for P-protein of the glycine cleavage | 0.43 | |
| 778 | M73760 | Mouse serine protienase, complete cds. | 0.43 | |
| 779 | Z36803 | H. sapiens pl (xs141) mRNA, 355 bp | 0.43 | |
| 780 | M33940 | Human Val-tRNA and Lys-tRNA genes. | 0.43 | |
| 781 | M96159 | Rattus norvegicus adenylyl cyclase type V mRNA, complete | 0.43 | |
| 782 | X54134 | Human HPTP epsilon mRNA for protein tyrosine | 0.43 | |
| 783 | Z73039 | S. cerevisiae chromosome VII reading frame ORF YGR254w | 0.43 | |
| 784 | AB002312 | Human mRNA for KIAA0314 gene, partial cds | 0.43 | |
| 785 | AB002312 | Human mRNA for KIAA0314 gene, partial cds | 0.43 | |
| 786 | U88667 | Human ATP binding cassette transporter (ABCR) mRNA, | 0.43 | |
| | | complete cds | | |
| 787 | U88667 | Human ATP binding cassette transporter (ABCR) mRNA, | 0.43 | |
| | | complete cds | | |
| 788 | Z65552 | H. sapiens CpG island DNA genomic Mse1 fragment, clone | 0.43 | |
| | | 46b12, reverse read cpg46b12.rt1a | | |
| 789 | X15599 | Phanerochaete chrysosporium LIP2 gene for lignin | 0.43 | |
| 790 | U22106 | Drosophila melanogaster dpoamine D1-like receptor mRNA, | 0.43 | |
| | | partial cds. | | |
| 791 | M13177 | Mouse transforming growth factor beta mRNA | 0.43 | |
| 792 | NM_002135.1 | Homo sapiens hormone receptor (growth factor-inducible | 0.42 | |
| | | nuclear protein N10) (HMR) mRNA > :: | | |
| | | gb\|L13740\|HUMTR3E Human TR3 orphan receptor mRNA. | | |
| 793 | U19617 | Mus musculus Ets-family transcription factor Elf-1 mRNA, | 0.42 | |
| | | complete cds | | |
| 794 | M60858 | Human nucleolin gene, complete cds. | 0.42 | |
| 795 | Z71179 | Caenorhabditis elegans cosmid F07D3, complete sequence | 0.42 | |
| | | [Caenorhabditis elegans] | | |
| 796 | Z11804 | D. discoideum ras gene | 0.42 | |
| 797 | L07572 | Equus caballus (clone GEN2-9) germline Ig lambda chain | 0.42 | |
| | | gene, J4-C4 region, last 2 exons. > :: gb\|L07573\|HRSIGLL | | |
| | | Equus caballus (clone HVL1) germline Ig lambda chain | | |
| 798 | L25637 | Xenopus laevis HNF-3beta gene, complete cds. | 0.42 | |
| 799 | AE000293 | Escherichia coli K-12 MG1655 section 183 of 400 of the | 0.42 | |
| | | complete genome | | |
| 800 | Z55829 | H. sapiens CpG island DNA genomic Mse1 fragment, clone | 0.42 | |
| | | 68a3, forward read cpg68a3.ft1a | | |
| 801 | Y11280 | A. vinelandii yoh-1, ibpB, cynR, leuC, leuD, leuB, asd, usg-1 | 0.42 | |
| 802 | Y08925 | P. falciparum aarp3 gene, exon | 0.42 | |
| 803 | X89961 | H. sapiens DNA for MCS gene | 0.42 | |
| 804 | X82330 | A. hypogaea chi2.2 gene for chitinase (class II) | 0.42 | |
| 804 | D89655 | Rattus norvegicus mRNA for scavenger receptor class B, | 0.42 | |
| | | complete cds | | |
| 806 | U70730 | Human SnoN2 mRNA, complete cds | 0.42 | |
| 807 | Y09922 | M. musculus flankin region of exon 1 of SEZ-6 gene | 0.42 | |
| | | including promotoer sequence | | |
| 808 | X57638 | Mouse mRNA for peroxisome proliferator activated receptor | 0.42 | |
| 809 | M33196 | Human Fc-epsilon-receptor gamma-chain gene, complete | 0.42 | |
| 810 | X13602 | Caldocellum saccharolyticum celB gene for | 0.42 | |
| | | cellobiohydrolase/endocellulase | | |
| 811 | X13602 | Caldocellum saccharolyticum celB gene for | 0.42 | |
| | | cellobiohydrolase/endocellulase | | |
| 812 | U23947 | Mycoplasma pulmonis putative lipoprotein (lipA), VsaB | 0.42 | |
| | | lipoprotein (vsaB), VsaC2 lipoprotein (vsaC2), VsaE2 | | |
| | | lipoprotein (vsaE2), VsaD lipoprotein (vsaD) genes, partial | | |
| | | cds, VsaA lipoprotein (vsaA) gene, complete cds, VsaC1 1 . . . | | |
| 813 | U37312 | Sus scrofa clone ARO34B cytochrome P450 aromatase | 0.42 | |
| | | mRNA, complete cds | | |
| 814 | U70998 | Phanerochaete chrysosporium manganese peroxidase | 0.42 | |
| | | isozyme 3 (mnp3) gene, complete cds | | |
| 815 | U70998 | Phanerochaete chrysosporium manganese peroxidase | 0.42 | |
| | | isozyme 3 (mnp3) gene, complete cds | | |
| 816 | X59379 | Mouse mRNA for amyloid beta precursor (protease nexin II) | 0.42 | |
| 817 | X54134 | Human HPTP epsilon mRNA for protein tyrosine | 0.42 | |
| 818 | Z32676 | H. sapiens x11 gene, promoter region | 0.42 | |
| 819 | U28938 | Rattus norvegicus protein tyrosine phosphatase D30 mRNA, | 0.42 | |
| | | complete cds | | |
| 820 | L41867 | Drosophila extra sex combs gene, exon 1-4, complete cds | 0.42 | |
| 821 | AF004659 | Andes virus G1 protein gene, partial cds | 0.42 | |
| 822 | U75187 | Arabidopsis thaliana germin-like protein (GPL1) mRNA, | 0.42 | |
| 823 | U27120 | Chlamydomonas reinhardtii ADP-ribosylation factor (ARF) | 0.42 | |
| | | mRNA, complete cds. | | |
| 824 | U41759 | Chlamydia psittaci RecJ recombination protein gene, partial | 0.41 | |
| | | cds and ORF2, ORF4, glutamyl-tRNA synthetase, outer | | |
| | | membrande protein 3 (omp3), outer membrane protein 2 | | |
| | | (omp2), and hypothetical sulfur-rich protein (srp) genes, . . . | | |
| 825 | D12716 | Candida maltosa ALK4 (CYP52D1) gene for n-alkane | 0.41 | |
| | | inducible cytochrome P-450, complete cds | | |
| 826 | J01261 | P. vulgaris lectin gene, complete cds. | 0.41 | |
| 827 | K00131 | mouse b2 repeat sequence from clone mm61. | 0.41 | |
| 828 | AF000949 | Canis familiaris keratin (KRT9) gene, complete cds | 0.41 | |
| 829 | U67580 | Methanococcus jannaschii section 122 of 150 of the | 0.41 | |
| 830 | X98568 | H. sapiens type X collagen gene | 0.41 | |
| 831 | X13595 | Bean DNA for glycine-rich cell wall protein GRP 1.0 | 0.41 | |
| 832 | Y13852 | Drosophila diplacantha clone D3 inactive Bari-1 family | 0.41 | |
| 833 | X53815 | Human G6PD gene for glucose-6-phosphate dehydrogenase | 0.41 | |
| 834 | L34069 | Bovine (clone p59K2) 3′,5′-cyclic nucleotide | 0.41 | |
| | | phosphodiesterase (BTPDE1A1) mRNA, complete cds.> :: | | |
| | | gb\|I30433\|I30433 Sequence 16 from U.S. Pat. No. 558077 > :: | | |
| | | gb\|I35677\|I35677 Sequence 16 from U.S. Pat. No. 5602019 > :: | | |
| 835 | U26425 | Human phospholipase C-beta-3 (PLCB3) gene, complete | 0.41 | |
| 836 | J04431 Homo sapiens transforming growth factor-beta 1 | 0.41 | | |
| | | gb\|I14074\|I14074 Sequence 1 from U.S. Pat. No. 5445941 | | |
| 837 | U32802 | Haemophilus influenzae Rd section 117 of 163 of the | 0.41 | |
| 838 | U24676 | Drosophila melanogaster twinstar (tsr) gene, complete cds | 0.41 | |
| 839 | U87793 | Bacillus thuringiensis kurstaki insecticidal delta-endotoxin | 0.41 | |
| | | CryIA(c) (cryIA(c)) gene, complete cds | | |
| 840 | L06446 | Mus musculus domesticus insulin-like growth factor 2 | 0.41 | |
| | | receptor (Igf2r) gene, exon 2 and partial cds | | |
| 841 | X06660 | Candida tropicalis POX9 gene for peroxisomal catalase | 0.41 | |
| | | (PXP-9) > :: dbj\|E01922\|E01922 DNA encoding catalase of | | |
| 842 | U05349 | Equine rotavirus glycoprotein VP7 mRNA, complete cds. | 0.41 | |
| 843 | L12058 | Clostridium sordellii 7-alpha-hydroxysteroid dehydrogenase | 0.41 | |
| | | gene, complete cds. | | |
| 844 | X71327 | M. musculus mRNA for MRE-binding transcription factor | 0.41 | |
| 845 | AE000534.1 | Helicobacter pylori 26695 section 12 of 134 of the complete | 0.41 | |
| 846 | L33727 | Mouse 43kDa acetylcholine receptor-associated protein | 0.41 | |
| | | (Rapsn) gene, exons 3-8 | | |
| 847 | Y11270 | E. histolytica INO1 gene | 0.41 | |
| 848 | L10390 | Camptotheca acuminata 3-hydroxy-3-methylglutaryl | 0.41 | |
| | | coenzyme A reductase gene, complete cds. | | |
| 849 | M34044 | Pig Na+/glucose cotransporter protein (SGLT1) mRNA, 3′ | 0.41 | |
| 850 | M20543 | Human skeletal alpha-actin gene, complete cds. | 0.41 | |
| 851 | U97192 | Caenorhabditis elegans cosmid C01F4 | 0.41 | |
| 852 | U65625 | Mus musculus strain BALB/c IgH C alpha gene, 3′ | 0.41 | |
| | | regulatory region enhancer E | | |
| 853 | X78344 | S. cerevisiae CAT8 gene | 0.41 | |
| 854 | Z26280 | H. sapiens isoform 1 gene for L-type calcium channel, exon | 0.41 | |
| 855 | Z23995 | H. sapiens (D1S469) DNA segment containing (CA) repeat; | 0.41 | |
| | | clone AFM280za5; single read | | |
| 856 | X51394 | Xenopus mRNA for APEG protein, containing a highly | 0.41 | |
| | | repetitive amino acid sequence | | |
| 857 | U72719 | Streptococcus agalactiae heat shock protein 70 cds | 0.41 | |
| 858 | M95673 | Saccharomyces cerevisiae pta1 gene, complete cds. | 0.41 | |
| 859 | U28938 | Rattus norvegicus protein tyrosine phosphatase D30 mRNA, | 0.41 | |
| | | complete cds | | |
| 860 | NM_001072.1 | Homo sapiens UDP glycosyltransferase 1 phenol UDP- | 0.41 | |
| | | glucuronosyltransferase (UDPGT) mRNA, complete cds. | 0.41 | |
| 861 | Z72884 | S. cerevisiae chromosome VII reading frame ORF YGR099w | 0.41 | |
| 862 | X96883 | H. sapiens spcDNA tetranucleoid and O-LTR like repeat, | 0.4 | |
| 863 | M14602 | Human myoglobin gene, exon 2. | 0.4 | |
| 864 | D00844 | Gallus gallus mRNA for virus activating protease, complete | 0.4 | |
| 865 | D49817 | Homo sapiens mRNA for 6-phosphofructo-2-kinase/fructose- | 0.4 | |
| | | 2,6-bisophosphatase, complete cds | | |
| 866 | L78742 | Homo sapiens (subclone 10_b11 from P1 H16) DNA | 0.4 | |
| 867 | X51772 | Rat DNA for calpain II large subunit (EC 3.4.22.17) (exon | 0.4 | |
| 868 | U09473 | Luxilus chrysocephalus chrysocephalus Meramec R., | 0.4 | |
| | | Jefferson Co., MO, USA, mitochondrion 12S rRNA and 16S | | |
| | | rRNA genes, partial sequence, and tRNA-Val gene, | | |
| 869 | X52614 | Maize chloroplast rps15 gene for ribosomal protein S15 | 0.4 | |
| 870 | U76524 | Sambucus nigra ribosome inactivating protein precursor | 0.4 | |
| | | mRNA, complete cds | | |
| 871 | M30467 | D. discoideum uridine diphosphoglucose pyrophosphorylase | 0.4 | |
| | | (UDPGP1) gene, 5′ end. | | |
| 872 | NM_002248.1 | Homo sapiens potassium intermediate/small conductance | 0.4 | |
| | | calcium-activated channel, subfamily N, member 1 | | |
| | | (KCNN1) mRNA > :: gb\|U69883\|HSU69883 Human | | |
| | | calcium-activated potassium channel hSK1 (SK) mRNA, | | |
| 873 | M81686 | Petuna hybrida Sx-protein | 0.4 | |
| 874 | S39508 | myelin P2 [mice, Genomic, 3694 nt] | | |
| 875 | U82966 | Oryza sativa Ca2+-ATPase gene, complete cds | 0.4 | |
| 876 | X16323 | Human mRNA, for hepatocyte growth factor (HGF) | 0.4 | |
| 877 | U31631 | Dictostelium discoideum class II apurinic/apyrimidinic (AP)- | 0.4 | |
| | | endonuclease (DdapeA) gene, complete cds | | |
| 878 | X60753 | P. sylvestris BBS gene for bibenzyl synthase | 0.4 | |
| 879 | V00711 | Mus musculus mitochondrial genome > :: | 0.4 | |
| | | gb\|J01420\|MUSMTCG Mouse mitochondrion, complete | | |
| 880 | U66274 | Rattus norvegicus neuropeptide Y5 receptor (NPYR5) | 0.4 | |
| | | mRNA, complete cds | | |
| 881 | X83673 | X. laevis SSB1 gene | 0.4 | |
| 882 | X14036 | Tomato cab-7 gene for type II chlorophyll a/b-binding | 0.4 | |
| 883 | U76524 | Sambucus nigra ribosome inactivating protein precursor | 0.4 | |
| | | mRNA, complete cds | | |
| 884 | X64467 | H. sapiens ALAD gene for porphobilinogen synthase | 0.4 | |
| 885 | L81673 | Homo sapiens (subclone 1_f8 from P1 H49) DNA sequence | 0.4 | |
| 886 | L36887 | Saccharomyces cerevisiae mitochondrion transfer RNA- | 0.4 | |
| | | Leu, Gln, Lys, Arg, Gly, Asp, Ser2, Arg2, Ala, Ile, Tyr, Asn | | |
| 887 | L24905 | Bombyx mori nuclear polyhedrosis virus hr5 gene. | 0.4 | |
| 888 | K00841 | human beta-tubulin pseudogene, clone 21-beta. | 0.4 | |
| 889 | K02819 | Rabbit MHC RLA region class I 19-1 gene, complete cds. | 0.4 | |
| 890 | L24905 | Bombyx mori nuclear polyhedrosis virus hr5 gene. | 0.4 | |
| 891 | X69491 | G. gallus gene for gonadotroyhin releasing hormone I | 0.4 | |
| 892 | X95887 | H. sapiens PLP gene, intron 1 | 0.4 | |
| 893 | Z96576 | H. sapiens telomeric DNA sequence, clone 3PTEL001, read | 0.4 | |
| | | 3PTELOO001.seq | | |
| 894 | Z16503 | H. sapiens (D1S196) DNA segment containing (CA) repeat; | 0.4 | |
| | | clone AFM063xg9; single read | | |
| 895 | M20572 | Mouse interleukin 6 (IL-6) gene, complete cds. | 0.4 | |
| 896 | AF022372 | Candida albicans proteinase (Kex2) gene, complete cds | 0.4 | |
| 897 | Z77964 | H. sapiens flow-sorted chromosome 6 HindIII fragment, | 0.4 | |
| 898 | Z58874 | H. sapiens CpG island DNA genomic Mse1 fragment, clone | 0.4 | |
| | | 110g9, reverse read cpg110g9.rt1a | | |
| 899 | X53422 | D. grimshawi s18, s15, s19 and s16 chorion protein genes | 0.4 | |
| 900 | NM_002435.1 | Homo sapiens mannose phosphate isomerase (MPI) mRNA > | 0.4 | |
| | | :: emb\|76057\|HSRPMI H. sapiens PMI1 mRNA for | | |
| | | phosphomannose isomerase | | |
| 901 | X05914 | D. virilis mitochondrial DNA for origin of replication, small | 0.4 | |
| | | mitochondrial ribosomal RNA, transer tRNAs tRNA-fMet, | | |
| | | tRNA-Gln and tRNA-Ile | | |
| 902 | Z95179 | G. gallus microsatellite DNA (LEI0290 (= EC11EO7)) | 0.4 | |
| 903 | X65627 | M. musculus mRNA TNZ2 for p68 RNA helicase | 0.4 | |
| 904 | U87940 | Salmonella typhimurium hydroxyethyl thiazole kinase (thiM) | 0.4 | |
| | | and HMP-P kinase (thiD) genes, complete cds | | |
| 905 | U15450 | Sus scrofa clone pvg13 Ig heavy chain variable VDJ region | 0.4 | |
| | | mRNA, partial cds. | | |
| 906 | M27902 | Rat cardiac specific sodium channel alpha-subunit mRNA, | 0.4 | |
| | | complete cds. | | |
| 907 | U22893 | Rattus norvegicus muscle Y-box protein YB2 mRNA, | 0.4 | |
| 908 | NM_003632.1 | Homo sapiens neurexin 4 (contactin associated protein) | 0.4 | |
| | | (NRXN4) mRNA > :: gb\|U87223\|HSU87223 Homo sapiens | | |
| | | contactin associated protein (Caspr) mRNA, complete cds | | |
| 909 | AF001504 | Homo sapiens myasthenia gravis autoantigen gravin mRNA, | 0.4 | |
| | | partial cds | | |
| 910 | AB002384 | Human mRNA for KIAA0386 gene, complete cds | 0.4 | |
| 911 | U40145 | Mus musculus Mdm2 (Mdm2) gene, complete cds. | 0.4 | |
| 912 | NM_002714.1 | Homo sapiens protein phosphatase 1, regulatory subunit 10 | 0.4 | |
| | | (PPP1R10) mRNA > :: emb\|Y13247\|HSFB19 Homo sapiens | | |
| 913 | Y09762 | Mokola virus genes encoding nucleoprotein, | 0.4 | |
| | | phosphoprotein, matrice rotein, glycoprotein and | | |
| 914 | U75187 | Arabidopsis thaliana germin-liile protein (GLP1) mRNA, | 0.4 | |
| 915 | Z82448 | R. prowazekii genomic DNA fragment (clone A503F) | 0.39 | |
| 916 | Z50864 | L. delbrueckii sp. lactis plasmid pWS58 DNA, complete | 0.39 | |
| 917 | K03196 | Human interferon-beta-3 gene. | 0.39 | |
| 918 | U66614 | Marinococcus halophilus L-2,4-diaminobutyric acid acetyl | 0.39 | |
| | | transferase (ectA) gene, L-2,4-diaminobutyric acid | | |
| | | transaminase (ectB) gene, ectoine synthase (ectC) gene, | | |
| | | putative transposase orfA gene, complete cds, and putative . . . | | |
| 919 | L48522 | Homo sapiens tuberin (TSC2) gene, exons 6, 7, and 8 | 0.39 | |
| 920 | Z68133 | Caenorhabditis elegans cosmid F23C11, complete sequence | 0.39 | |
| | | [Caenorhabditis elegans] | | |
| 921 | U60973 | Candida albicans oligopeptide transporter (OPT1) gene, | 0.39 | |
| 922 | L31398 | Mus musculus dynamin (UDnm) mRNA, complete cds. | 0.39 | |
| 923 | X15966 | Rabbit DNA for L1Oc4 repeat | 0.39 | |
| 924 | Z95122 | Caenorhabditis elegans cosmid VZC374L, complete | 0.39 | |
| | | sequence [Caenorhabditis elegans] | | |
| 925 | X55036 | M. musculus long mosaic repeated sequence (LMRS) | 0.39 | |
| 926 | U35640 | Arabidopsis thaliana thioredoxin h (TRX3) gene, complete | 0.39 | |
| 927 | V00094 | Fibroin gene intervening sequence from Bombyx mori (silk | 0.39 | |
| | | moth), with 5′ flank and partial coding sequence | | |
| 928 | U47687 | Streptococcus pneumoniae immunoglobulin A1 protease | 0.39 | |
| | | (iga) gene, complete cds | | |
| 929 | Z93381 | Caenorhabditis elegans cosmid F28G4, complete sequence | 0.39 | |
| | | [Caenorhabditis elegans] | | |
| 930 | U76524 | Sambucus nigra ribosome inactivating protein precursor | 0.39 | |
| | | mRNA, complete cds | | |
| 931 | X71600 | H. sapiens DNA sequence of polymorphic pentanucleotide | 0.39 | |
| 932 | NM_000958.1 | Homo sapiens prostaglandin E receptor 4 sapiens | 0.39 | |
| | | prostaglandin E2 receptor EP2 subtype mRNA, complete | | |
| | | cds. > :: gb\|I36298\|I36298 Sequence 1 from U.S. Pat. No. | | |
| 933 | Y07648 | A. thaliana nit2 gene, nit1 gene and nit3 gene | 0.39 | |
| 934 | Y13852 | Drosophila diplacantha clone D3 inactive Bari-1 family | 0.39 | |
| 935 | M57851 | Human S protein-alpha (PS-alpha) gene, exon 13. | 0.39 | |
| 936 | X52270 | Maize chloroplast 3′part of rpoC2 gene, rps2 gene, atpI gene | 0.39 | |
| | | and 5′part of atpH gene | | |
| 937 | U50948 | Rattus norvegicus taste bud receptor protein TB 567 (TB | 0.39 | |
| | | 567) gene, complete cds | | |
| 938 | X60435 | H. sapiens gene PACAP for pituitary adenylate cyclase | 0.39 | |
| | | activating polypeptide | | |
| 939 | U12972 | Tetrahymena thermophila CU428.1VII micronuclear R | 0.39 | |
| 940 | X54709 | Kluyveromyces lactis BiP gene for BiP/GRP78 | 0.39 | |
| 941 | Z73360 | Human DNA sequence from cosmid 92M18, BRCA2 gene | 0.39 | |
| | | region chromosome 13q12-13 | | |
| 942 | L13841 | Plasmid pX01 (from Bacillus anthracis UM23-1) trans- | 0.39 | |
| | | acting positive regulator (Atx A) gene, complete cds. | | |
| 943 | Z72554 | S. cerevisiae chromosome VII reading frame ORF YGL032c | 0.39 | |
| 944 | L24905 | Bombyx mori nuclear polyhedrosis virus hr5 gene. | 0.39 | |
| 945 | Z11486 | Pinus strobus L. mRNA for pine globulin-1 | 0.39 | |
| 946 | X70675 | M. musculus gene for MERANTES protein | 0.39 | |
| 947 | AF010537 | Plasmodium falciparum microsatellite TA3 sequence | 0.39 | |
| 948 | X78219 | D. melanogaster (Barton) SED5 mRNA | 0.39 | |
| 949 | D42055 | Human mRNA for KIAA0093 gene, partial cds | 0.39 | |
| 950 | Z30698 | Rinderpest virus (RBOK) RNA for RNA polyinerase (L) | 0.39 | |
| 951 | M64715 | Plasmodium falciparum DNA polymerase-delta gene, | 0.39 | |
| 952 | Z32774 | H. sapiens gene for N-methyl-D-aspartate receptor R1 exons | 0.39 | |
| 953 | U70998 | Phanerochaete chrysosporium manganese peroxidase | 0.39 | |
| | | isozyme 3 (mnp3) gene, complete cds | | |
| 954 | Z72884 | S. cerevisiae chromosome VII reading frame ORF YGR099w | 0.39 | |
| 955 | S78378 | Oct-4 (t12 haplotype) [mice, Genomic, 1191 nt, segment 3 | 0.39 | |
| 956 | U78098 | Pyrococcus furiosus ribonucleotide reductase (rnr) gene, | 0.38 | |
| | | complete cds | | |
| 957 | U78857 | Rattus norvegicus protein serine/threonine kinase CPG167 | 0.38 | |
| | | (cpg16) mRNA, complete cds | | |
| 958 | Z54096 | S. pombe chromosome I cosmid c13A11 | 0.38 | |
| 959 | Z72778 | S. cerevisiae chromosome VII reading frame ORF YGL256w | 0.38 | |
| 960 | D14718 | Human chromosomal protein HMG1 related gene | 0.38 | |
| 961 | U09138 | Mus musculus peroxisome proliferator activated protein- | 0.38 | |
| | | gamma-2 mRNA, complete cds. | | |
| 962 | U32818 | Haemophilus influenze Rd section 133 of 163 of the | 0.38 | |
| 963 | X89398 | H. sapiens ung gene for uracil DNA-glycosylase | 0.38 | |
| 964 | X69440 | N. tabacum NTP303 gene | 0.38 | |
| 965 | U62660 | Toxoplasma gondii micronemal protein MIC2, complete cds | 0.38 | |
| 966 | AE000042 | Mycoplasma pneumoniae section 42 of 63 of the complete | 0.38 | |
| 967 | V00190 | Gene for small nuclear RNA D2 of slime mold (D. | 0.38 | |
| 968 | X98568 | H. sapiens type X collagen gene | 0.38 | |
| 969 | U54776 | Human NTT gene, L1, Alu, and MER 38 repeat regions | 0.38 | |
| 970 | Z72888 | S. cerevisiae chromosome VII reading frame ORF YGR103w | 0.38 | |
| 971 | U97128 | Plasmodium falciparum HMG-like protein Pf16 (Pf16) | 0.38 | |
| | | mRNA, partial cds | | |
| 972 | J02846 | Human tissue fact or gene, complete cds. | 0.38 | |
| 973 | J00803 | Sheep corticotropin-releasing factor (CRF) mRNA, complete | 0.38 | |
| 974 | X04310 | Rat thymocyte mRNA for 37K of CD8 antigen | 0.38 | |
| 975 | Y13129 | Rickettsia akari fmt gene, partial | 0.38 | |
| 976 | X95099 | C. albicans mRNA for neutral trehalase | 0.38 | |
| 977 | D83948 | Rat adult liver mRNA for S1-1 protein, complete cds | 0.38 | |
| 978 | Z84722 | Human DNA sequence from cosmid GG4 from a contig | 0.38 | |
| | | from the tip of the short arm of chromosome 16, spanning | | |
| 979 | U40604 | Listeria monocytogenes ClpC ATPase (mec) gene, complete | 0.38 | |
| 980 | Z92808 | Caenorhahditis elegans cosmid M04C3, complete sequence | 0.38 | |
| | | [Caenorhabditis elegans] | | |
| 981 | X91882 | Z. mays ZEMa gene | 0.38 | |
| 982 | M26394 | M. sexta larval cuticle protein (LCP-14) gene, exon 1. | 0.38 | |
| 983 | U32021 | Gossypium tomentosum 5S ribosomal RNA gene and non- | 0.38 | |
| | | transcribed spacer, clone 11 | | |
| 984 | X54061 | D. melanogaster mRNA coding for a 205K microtubule- | 0.38 | |
| | | associated protein (MAP) | | |
| 985 | X14612 | Chicken c-myb proto-oncogene 5′region exons 1-5 | 0.38 | |
| 986 | U12594 | Sus scrofa immunoglobulin alpha heavy chain constant | 0.38 | |
| | | region (IgA C alpha) mRNA, partial cds. | | |
| 987 | U64841 | Caenorhabditis elegans cosmid ZC142. | 0.38 | |
| 988 | M93148 | Mouse homeobox protein (Hox-1.11) gene, complete cds. | 0.38 | |
| 989 | X14049 | Canis familiaris RDC4 mRNA for G protein-coupled | 0.38 | |
| 990 | X94616 | M. musculus mRNA for glycogen synthase | 0.38 | |
| 991 S78378 | Oct-3 (t12 haplotype) [mice, Genomic, 1191 nt, segment 3 | 0.38 | | |
| 992 | D50091 | Drosophila ezoana G-3-P dehydrogenase | 0.37 | |
| 993 | Y11874 | M. musculus uPA gene, promoter sequence | 0.37 | |
| 994 | X63628 | S. pombe MFm2 gene | 0.37 | |
| 995 | Z69652 | Human DNA sequence from cosmid L75B9, Huntington's | 0.37 | |
| | | Disease Region, chromosome 4p16.3 | | |
| 996 | Y08305 | L. esculentum lap17.1a gene, promoter region and CDS | 0.37 | |
| 997 | U64453 | Human ELK1 pseudogene (ELK2) and immunoglobulin | 0.37 | |
| | | heavy chain gamma pseudogene (IGHGP) | | |
| 998 | X82286 | H. sapiens Fas, Apo-1 gene (exon IX) | 0.37 | |
| 999 | Z48231 | E. coli IncQ plasmid pIE639 sat3 gene and aphA gene | 0.37 | |
| 1000 | L36679 | Amycolatopsis methanolica plasmid pMEA300 stf gene, | 0.37 | |
| 1001 | X95275 | P. falciparum complete gene map of plastid-like DNA | 0.37 | |
| 1002 | Z58565 | H. sapiens CpG island DNA genomic Mse1 fragment, clone | 0.37 | |
| | | 44a5 forward read cpg44a5ft1a | | |
| 1003 | L14625 | Arcobacter skirrowi 16S ribosomal RNA. | 0.37 | |
| 1004 | U72236 | Dictyostelium discoideum ModA (modA) gene, complete | 0.37 | |
| 1005 | X73940 | B. taurus microsatellite DNA INRA190 | 0.37 | |
| 1006 | L06092 | Mesocricetus auratus acidic fibroblast growth factor gene, 5′ | 0.37 | |
| | | flank and non-coding exon 1. | | |
| 1007 | X98918 | Human rotavirus gene encoding outer capsid glycoprotein | 0.37 | |
| 1008 | X14448 | Human GLA gene for alpha-D-galactosidase A (EC | 0.37 | |
| 1009 | AF003533 | Homo sapiens cytosolic phagocyte oxidase protein | 0.37 | |
| 1010 | X67711 | O. sativa hsp70 gene for heat shock protein 70 | 0.37 | |
| 1011 | Z33465 | B. occidentalis gene for 18S rRNA | 0.37 | |
| 1012 | Z12652 | R. norvegicus upstream of gene for catechol | 0.37 | |
| 1013 | U45934 | Human papillomavirus type 68, isolate IS362, major capsid | 0.37 | |
| | | protein L1 (L1) gene, partial cds | | |
| 1014 | U17084 | Muman neurofibromin (NF1) gene, promoter region and | 0.37 | |
| 1015 | X95668 | B. burgdorferi gidA, gidB and moxR genes | 0.37 | |
| 1016 | X58541 | Mink mRNA for plasminogen activator inhibitor type 1 | 0.37 | |
| 1017 | U48706 | Dictyostelium discoideum small aggregate formation | 0.37 | |
| 1018 | M77176 | H. sapiens carbonic anhydrase II (CAII) gene, exons 1 and 2. | 0.37 | |
| 1019 | Z72888 | S. cerevisiae chromosome VII reading frame ORF YGR103w | 0.37 | |
| 1020 | Y13234 | Chironomus tentans mRNA for chitinase, 1695 bp | 0.37 | |
| 1021 | AF004166 | Lycopersicon pennellii 2-isopropylmalate synthase (lp- | 0.37 | |
| | | ipmsb) mRNA, complete cds | | |
| 1022 | D85240 | Aspergillus niger DNA for isopullulanase precursor, | 0.37 | |
| 1023 | Y00684 | Rabbit mRNA for gamma-subunit of phosphorylase kinase | 0.37 | |
| | | (EC 2.7.1.38) | | |
| 1024 | X54016 | D. discoideum mRNA for 24 kDa protein, homologous to C- | 0.37 | |
| | | terminal repeat sequence of rhodopsin and synaptophysin | | |
| 1025 | M22015 | Influenza virus type C (C/JJ/50) nonstructural | 0.37 | |
| 1026 | M62798 | pi F. ferrugineum 16S ribosomal RNA. | 0.37 | |
| 1027 | X76652 | M. musculus mRNA for 3f8 | 0.37 | |
| 1028 | X56047 | P. chrysosporium trpC gene for trifunctional polypeptide | 0.37 | |
| 1029 | Z74896 | S. cerevisiae chromosome XV reading frame ORF YOL154w | 0.37 | |
| 1030 | U20238 | Mus musculus GTPase-activating protein GAPIII mRNA, | 0.37 | |
| | | complete cds. | | |
| 1031 | Z54240 | L. plantarum pyrimidine biosynthetic operon genes | 0.36 | |
| 1032 | X54742 | Nicotiana plumbaginifolia beta-(1,3)-glucanase gene for a | 0.36 | |
| | | vacuolar isoform | | |
| 1033 | AF001415 | Arabidopsis thaliana 14-3-3-like protein GF14 upsilon | 0.36 | |
| | | (GRF5) gene, complete cds | | |
| 1034 | X77801 | S. beecheyi genomic DNA with direct repeats and integrated | 0.36 | |
| | | Hepatitis B virus | | |
| 1035 | Z84431 | D. ligulata internal transcribed spacer 2 DNA | 0.36 | |
| 1036 | X60753 | P. sylvestris BBS gene for bibenzyl synthase | 0.36 | |
| 1037 | D28503 | Clostridium josui hemA, hemC, hemD and hemB genes of | 0.36 | |
| | | porphyrin biosynthesis | | |
| 1038 | X66131 | R. americana mRNA for elongation factor 1-alpha | 0.36 | |
| 1039 | X69601 | B. burgdorferi p93 gene (partial) | 0.36 | |
| 1040 | M83758 | Mytilus edulis mitochondrial cytochrome oxidase subunit II | 0.36 | |
| | | (COII) gene, 3′ end and NADH dehydrogenase subunit 1 | | |
| 1041 | Z27392 | G. raimondii (D61) copia-like reverse transcriptase | 0.36 | |
| 1042 | U28832 | Infectious laryngotracheitis virus US10, US2, protein kinase, | 0.36 | |
| | | UL47, glycoprotein G, ORF5, glycoprotein D, glycoprotein | | |
| | | I, glycoprotein E, ORF9 genes, complete cds | | |
| 1043 | AB002384 | Human mRNA for KIAA0386 gene, complete cds | 0.36 | |
| 1044 | M22345 | Mouse endogenous provirus gag, pol, and env region DNA. | 0.36 | |
| 1045 | U16850 | Human calmodulin-I (CALM1) mRNA, 3′UTR, partial | 0.36 | |
| 1046 | M19197 | Dengue virus type 2 (S1 vaccine strain), complete genome. | 0.36 | |
| 1047 | Z49535 | S. cerevisiae chromosome X reading frame ORF YJR035w | 0.36 | |
| 1048 | Z74369 | S. cerevisiae chromosome IV reading frame ORF YDR073w | 0.36 | |
| 1049 | S78378 | Oct-4 (t12 haplotype) [mice, Genomic, 1191 nt, segment 3 | 0.36 | |
| 1050 | X60686 | B. taurus mRNA for transglutaminase | 0.36 | |
| 1051 | NM_001670.1 | Homo sapiens armadillo repeat gene deleted in velo-cardio- | 0.35 | |
| | | facial syndrome (ARVCF) mRNA > :: | | |
| | | gb\|U51269\|HSU51269 Human armadillo repeat protein | | |
| 1052 | X63149 | Y. enterocolitica ampC and ampR genes for beta-lactamase | 0.35 | |
| | | and AmpR regulatory protein | | |
| 1053 | L25779 | Kluyveromyces lactis (HAP3) gene, complete cds, | 0.35 | |
| 1054 | U77368 | Listeria monocytogenes internalin (in1C2), in1D, and in1E | 0.35 | |
| | | genes, complete cds | | |
| 1055 | M62750 | Homo sapiens intergenic locus pYNZ32 variable number | 0.35 | |
| | | tandem repeat (VNTR) sequence associated with Huntington | | |
| 1056 | K03073 | Slime mold (D. discoideum) mRNA complementary to the | 0.35 | |
| | | right inverted terminal repeat of DIRS-1, clone pLZ12. | | |
| 1057 | U78025 | Sus scrofa microsatellite S0399 sequence | 0.35 | |
| 1058 | M17478 | Drosophila melanogaster transformer (tra) gene, complete | 0.35 | |
| | | cds, and non-sex-specific transformer processed pseudogene | | |
| 1059 | L09708 | Homo sapiens complement component 2 (C2) gene allele b, | 0.35 | |
| | | exons 10 through 18 and complete cds | | |
| 1060 | Z16708 | H. sapiens (D6S275) DNA segment containing | 0.35 | |
| 1061 | M74201 | Rabbit cytochrome P450IIC4 (CYP2C4) gene, exons 2 and | 0.35 | |
| 1062 | U63634 | Mesembryanthemum crystallinum inositol methyltransferase | 0.35 | |
| | | (Imt1) gene, complete cds | | |
| 1063 | U73126 | Oryctolagus cuniculus calcitonin receptor isoform mRNA, | 0.35 | |
| | | complete cds | | |
| 1064 | X52614 | Maize chloroplast rps15 gene for ribosomal protein S15 | 0.35 | |
| 1065 | X52614 | Maize chloroplast rps15 gene for ribosomal protein S15 | 0.35 | |
| 1066 | X57520 | L. micdadei DNA for gro ELS operon | 0.35 | |
| 1067 | D38574 | Pyrodictium occultum polB gene for DNA polymerase II | 0.35 | |
| | | (family B) (alpha-like DNA polymerase), complete cds | | |
| 1068 | Z47075 | Caenorhabditis elegans cosmid E02H1, complete sequence | 0.35 | |
| | | [Caenorhabditis elegans] | | |
| 1069 | Y10686 | C. capreolus DNA, tandem repeat region | 0.35 | |
| 1070 | U17771 | Blaberus giganteus mitochondrion 16S ribosomal RNA, | 0.35 | |
| | | partial sequence | | |
| 1071 | U12186 | Oryctolagus cuniculus sodium/dicarboxylate cotransporter | 0.35 | |
| | | mRNA, partial cds. | | |
| 1072 | L25677 | Schizosaccharomyces pombe cdc42+ (Cdc42p) gene, | 0.35 | |
| 1073 | X63628 | S. pombe MFm2 gene | 0.35 | |
| 1074 | D67071 | Rat gene for regucalcin, exon1 (non-coding exon) | 0.35 | |
| 1075 | X93218 | Impatiens necrotic spot virus L gene | 0.35 | |
| 1076 | U16301 | Human lysyl hydroxylase (PLOD) gene, intron 15, complete | 0.35 | |
| 1077 | M37277 | Human Ig germline H-chain D-region genes, partial cds. | 0.35 | |
| 1078 | X14448 | Human GLA gene for alpha-D-galactosidase A (EC | 0.34 | |
| 1079 | X79930 | O. gibsoni Tbb gene | 0.34 | |
| 1080 | Z37352 | H. sapiens rearranged immunoglobulin lambda light chain V- | 0.34 | |
| | | region (V1-3 | ) | |
| 1081 | X59013 | Rat mRNA for trypsin V b-form | 0.34 | |
| 1082 | X90761 | Homo sapiens hHa2 gene | 0.33 | |
| 1083 | L23498 | Bovine microsatellite repeats | 0.33 | |
| 1084 | Z18361 | O. aries trichohyalin | 0.33 | |
| 1085 | L18785 | Plasmodium falciparum DNA polymerase alpha (Pol alpha) | 0.33 | |
| 1086 | X96685 | B. burgdorferi cell division genes | 0.33 | |
| 1087 | M58509 | Human adrenodoxin reductase gene, exons 3 to 12 | 0.33 | |
| 1088 | X70809 | C. reptans gene for 18S ribosomal RNA | 0.32 | |
| 1089 | D38116 | Pygmy chimpanzee mitochondrial DNA, complete sequence | 0.32 | |
| 1090 | U00310 | Human immunodeficiency virus type 1 isolate 583-78 | 0.31 | |
| | | envelope glycoprotein, V3 region (env) gene, partial cds. | | |
| 1091 | L35670 | Homo sapiens (subclone H8 10_g5 from P1 35 H5 C8) DNA | 0.3 | |
| 1092 | M63960 | Human protein phosphatase-1 catalytic subunit mRNA, | 0.3 | |
| 1093 | Z50747 | C. psittaci DNA for kdsA, dsk1 and dsk2 genes | 0.22 | |
| 1094 | M80515 | Streptococcus pneumoniae uvs402 protein gene, complete | 0.22 | |
| 1095 | M98776 | Human keratin 1 gene, complete cds | 0.22 | |
| 1096 | X66313 | H. sapiens GLUDP2 gene (exon 2) | 0.21 | |
| 1097 | AB001025 | Homo sapiens mRNA for brain ryanodine receptor, complete | 0.21 | |
| 1098 | AE000956 | Archaeogiobus fulgidus section 151 of 172 of the complete | 0.21 | |
| 1099 | Z46268 | Simian herpesvirus B DNA for glycoprotein G | 0.21 | |
| 1100 | X99403.1 | N. tabacum mRNA for defensin | 0.21 | |
| 1101 | U20335 | Drosophila pseudoobscura EP arrangement, BC p430#44 | 0.21 | |
| | | pAP5 clone alpha-amylase (amy1) gene, complete cds. > :: | | |
| | | gb\|U20337\|DPU20337 Drosophila pseudoobscura HI | | |
| | | arrangernent, Mex a-176x pAP9 clone alpha-amylase (amy1) | | |
| 1102 | X04077 | Potato patatin pseudogene (SB6B) | 0.2 | |
| 1103 | L11236 | Rattus rattus mRNA sequence. | 0.2 | |
| 1104 | X63547 | H. sapiens mRNA for tre oncogene (clone 213) > :: | 0.2 | |
| | | gb\|I76205\|I76205 Sequence 9 from U.S. Pat. No. 5691147 | | |
| 1105 | M69051 | Human liver glucokinase (ATP:D-hexose 6- | 0.2 | |
| | | phosphotransferase) mRNA, complete cds. | | |
| 1106 | U14950 | Rattus norvegicus synapse-associated protein 97 mRNA, | 0.2 | |
| | | complete cds. | | |
| 1107 | NM_002583.1 | Homo sapiens PRKC, apoptosis, WT1, regulator apoptosis | 0.2 | |
| | | response protein par-4 mRNA, complete cds | | |
| 1108 | X66313 | H. sapiens GLUDP2 gene (exon 2) | 0.2 | |
| 1109 | U48485 | Human skeletal muscle ryanodine receptor gene | 0.2 | |
| 1110 | AF018116 | Dendroica pityophila cytochrome oxidase I protein, partial | 0.2 | |
| 1111 | X87108 | S. cerevisiae CKI3 gene | 0.19 | |
| 1112 | M55249 | E. coli retron Ec67 DNA encoding reverse transcriptase and | 0.19 | |
| | | Dam methylase functions. | | |
| 1113 | L06898 | Actinomyces viscosus sialidase (nanH) gene, complete cds. | 0.19 | |
| 1114 | Y13631 | Clostridium botulinum P-21, P-47 ntnh, bonT genes | 0.18 | |
| 1115 | L24904 | Bombyx mori nuclear polyhedrosis virus hr4R gene. | 0.18 | |
| 1116 | M85049 | Drosophila melanogaster brahma protein mRNA, complete | 0.18 | |
| 1117 | X60777 | Shigella dysenteriae ipabCD genes | 0.17 | |
| 1118 | L42936 | Pseudoxycheila bipustulata mitochondrial large subunit 16S | 0.17 | |
| | | ribosomal RNA (16S rRNA) gene | | |
| 1119 | AE001359 | Chlamydia trachomatis section 86 of 87 of the complete | 0.17 | |
| 1120 | K00899 | yeast(s. cerevisiae) histone 3 gene (h3) fused with e. coli lacz | 0.17 | |
| | | gene and promoter, clone prm115. | | |
| 1121 | M83994 | Staphylococcus aureus prolipoprotein signal peptidase (lsp) | 0.17 | |
| | | gene, complete cds. | | |
| 1122 | Z28050 | S. cerevisiae XI reading frame ORF YKL050c | 0.16 | |
| 1123 | L23971 | Mus musculus fragile X mental retardation syndrome protein | 0.16 | |
| | | (Fmr1) (homologue) mRNA, complete cds. | | |
| 1124 | Z98560 | S. pombe chromosome I cosmid c4C5 | 0.15 | |
| 1125 | U89258 | Oxytricha fallax ‘D, D35E’ type transposase gene, partial cds | 0.15 | |
| 1126 | X62522 | R. norvegicus gene for CNS-myelin proteolipid protein (exon | 0.15 | |
| 1127 | X74504 | M. musculus T10 mRNA | 0.15 | |
| 1128 | D50931 | Human mRNA for KIAA0141 gene, complete cds | 0.15 | |
| 1129 | Z48951 | S. cerevisiae chromosome XVI cosmid 9723 | 0.15 | |
| 1130 | L12395 | Brassica napus small GTP-binding protein (bra) mRNA, | 0.15 | |
| 1131 | U39367 | Blepharoneura manchesteri 16S ribosomal RNA gene, | 0.15 | |
| | | mitochondrial gene encoding mitochondrial RNA, partial | | |
| 1132 | D50357 | Leptocarabus procerulus mitochondrial DNA for NADH | 0.15 | |
| | | dehydrogenase subunit 5, partial cds | | |
| 1133 | L35272 | Glycine max heat shock protein (SB100) complete | 0.15 | |
| 1134 | U63641 | Ligionella pneumophila rpoD operon LporfX, LpdnaG, and | 0.15 | |
| | | LprpoD genes, complete cds | | |
| 1135 | X72186 | H. sapiens C6 gene, exons 10, 11 and 12 | 0.15 | |
| 1136 | D45893 | Neurospora crassa acr-2 DNA, complete cds | 0.15 | |
| 1137 | L47106 | Neurospora crassa kinesin (NKIN) mRNA, complete cds | 0.15 | |
| 1137 | L04457 | Phytophthora megasperma mitochondrial ORF152, complete | 0.15 | |
| | | cds, cytochrome c oxidase subunit I (cox1) gene, complete | | |
| | | cds, cytochrome c oxidase subunit II | | |
| 1139 | L04457 | Phytophthora megasperma mitochondrial ORF152, complete | 0.15 | |
| | | cds, cytochrome c oxidase subunit I (cox1) gene, complete | | |
| | | cds, cytochrome c oxidase subunit II | | |
| 1140 | U12539 | Schizosaccharomyces pombe scd2 (scd2) gene, complete | 0.15 | |
| 1141 | U12539 | Schizosaccharomyces pombe scd2 (scd2) gene, complete | 0.15 | |
| 1142 | AB001901 | Homo sapiens PACE4 gene, exon 4-7 | 0.15 | |
| 1143 | M97550 | Maize sucrose phosphate synthase mRNA, complete cds. > :: | 0.15 | |
| | | emb\|A17878.1\|A17878 Synthetic SPS sequence > | | |
| 1144 | AB001901 | Homo sapiens PACE4 gene, exon 4-7 | 0.15 | |
| 1145 | X07438 | Human DNA for cellular retinol binding protein | 0.14 | |
| 1146 | Z57339 | H. sapiens CpG island DNA genomic Mse1 fragment, clone | 0.14 | |
| | | 171g3, reverse read cpg171g3.rt1a | | |
| 1l47 | X05034 | Rat C2A gene for prostatic binding protein (PBP) | 0.14 | |
| 1148 | U55712 | Human ataxia-telangiectasia (ATM) gene, exon 12 | 0.14 | |
| 1149 | M20273 | Human hepatitis virus type A RNA, complete genome. | 0.14 | |
| 1150 | J01764 | Plasmid pT181, complete genome. | 0.14 | |
| 1151 | X61109 | G. gorilla beta-globingene | 0.14 | |
| 1152 | Z30587 | S. hominis (972) genes for potential ABC transporter and | 0.14 | |
| | | potential membrane spanning protein | | |
| 1153 | U60400 | Babanki virus polymerase nsP4 gene, partial cds virus | 0.14 | |
| | | polymerase nsP4 gene, partial cds | | |
| 1154 | U67478 | Methanococcus jannaschii section 20 of 150 of the complete | 0.14 | |
| 1155 | Z71641 | S. cerevisiae chromosome XIV reading frame ORF | 0.14 | |
| 1156 | X53522 | M. musculus VL1 gene (1G-5) | 0.14 | |
| 1157 | Z21753 | O. tanganicae mitochondrion genes for tRNA-Thr | 0.14 | |
| 1158 | L33841 | Carthamus tinctorius glycerol-3-phosphate acyltransferase | 0.14 | |
| | | mRNA, complete cds. | | |
| 1159 | AF003277 | Glossiphonia complanata cytochrome c oxidase subunit I | 0.14 | |
| | | (COI) gene, partial cds | | |
| 1160 | U89035 | Oxytricha fallax transposon TBE1, insertion fal6, 42 kDa | 0.14 | |
| | | transposase gene, partial cds | | |
| 1161 | L36923 | Streptococcus pneumomae beta-N-acetylhexosaminidase | 0.14 | |
| | | (strH) gene, complete cds | | |
| 1162 | U64830 | Dictyostelium discoideum AX2 protein tyrosine kinase gene, | 0.14 | |
| | | complete cds. | | |
| 1163 | U43082 | Zea mays T cytoplasm male sterility restorer factor 2 (rf2) | 0.14 | |
| | | mRNA, complete cds | | |
| 1164 | Z26492 | T. repens TrMT1A mRNA for metallothionein-like protein | 0.14 | |
| 1165 | X83683 | V. sativa mRNA for early nodulin 40 | 0.14 | |
| 1166 | D63861 | Homo sapiens DNA for cyclophilin 40, complete cds | 0.14 | |
| 1167 | D86594 | Japanese jack bean clone CgHMGY1 DNA for high mobility | 0.14 | |
| | | group protein, complete cds | | |
| 1168 | Z18859 | H. sapiens gene for cone transducin alpha subunit | 0.14 | |
| 1169 | L28038 | Trypanosoma rangeli kinetoplast DNA sequence with | 0.14 | |
| | | conserved sequence blocks | | |
| 1170 | U72057 | Mus musculus chloride channel regulator (Ic1n) gene, exon 1 | 0.14 | |
| 1171 | AF003836 | Mesocricetus auratus isopentenyl diphosphate:dimethylallyl | 0.14 | |
| | | diphosphate isomerase mRNA, complete cds | | |
| 1172 | X78422 | A. thaliana HYP1 mRNA | 0.14 | |
| 1173 | U28042 | Human DEAD box RNA helicase-like protein mRNA, | 0.14 | |
| 1174 | U01070 | Mycoplasma flocculare Ms42 5S rRNA gene. | 0.14 | |
| 1175 | U54776 | Human NTT gene, L1, Alu, and MER 38 repeat regions | 0.14 | |
| 1176 | U40375 | Mus musculus Supt6h mRNA, complete cds | 0.14 | |
| 1177 | X71083 | G. max gene for coproporphyrinogen oxidase | 0.14 | |
| 1178 | D86253 | Human MHC (HLA) DRB intron 1 DNA, partial sequence | 0.14 | |
| 1179 | M55002 | Synechococcus sp. 6-phosphogluconate dehydrogenase | 0.14 | |
| | | gene, complete cds. | | |
| 1180 | X14036 | Tomato cab-7 gene for type II chlorophyll a/b-binding | 0.14 | |
| 1181 | L35272 | Glycine max heat shock protein (SB100) mRNA, complete | 0.14 | |
| 1182 | D50931 | Human mRNA for KIAA0141 gene, complete cds | 0.14 | |
| 1183 | L35272 | Glycine max heat shock protein (SB100) mRNA, complete | 0.14 | |
| 1184 | X05034 | Rat C2A gene for prostatic binding protein (PBP) | 0.14 | |
| 1185 | X89553 | Echovirus 26 DNA for VP2 capsid protein gene | 0.14 | |
| 1186 | Z77656 | Caenorhabditis elegans cosmid F07B10, complete sequence | 0.14 | |
| | | [Caenorhabditis elegans] | | |
| 1187 | Z84739 | H. sapiens flow-sorted chromosome 6 HindIII fragment, | 0.14 | |
| 1188 | L34219 | Homo sapiens retinaldehyde-binding protein (CRALBP) | 0.14 | |
| | | gene, complete cds. | | |
| 1189 | M30168 | D. melanogaster nested repetitive sequences F and G, | 0.14 | |
| 1190 | U07260 | Perissodus microlepis T32a mitochondrion NADH | 0.14 | |
| | | dehydrogenase subunit 2 gene, complete cds | | |
| 1191 | X15308 | H. sapiens NF-H gene,exon 3 | 0.14 | |
| 1192 | J02763 | Human calcyclin gene, complete cds. | 0.14 | |
| 1193 | M76175 | Mouse elongation factor 2 pseudogene, complete cds. | 0.14 | |
| 1194 | X67735 M. musculus mas gene for Mas proto-oncogene | 0.14 | | |
| 1195 | NM_002816.1 | Homo sapiens proteasome (prosome, macropain) 26S | 0.14 | |
| | | subunit, non-ATPase, 12 (PSMD12) mRNA > :: | | |
| | | dbj\|AB003103\|AB003103 Homo sapiens mRNA for 26S | | |
| 1196 | U20754 | Felis catus chromosome D2 mtDNA tandem repeat Numt | 0.14 | |
| 1197 | L00727 | Human myotonin protein kianse (Mt-PK) gene, complete | 0.14 | |
| | | cds. > :: gb\|I25678\|I25678 Sequence 10 from U.S. Pat. No. | | |
| 1198 | M57750 | S. pombe cut2+ gene, complete cds. | 0.14 | |
| 1199 | X03000 | Adenovirus type7 genome left end (0.0 to 31%) | 0.14 | |
| 1200 | NM_002911.1 | Homo sapiens regulator of nonsense transcripts 1 (RENT1) | 0.14 | |
| | | mRNA > :: gb\|U65533\|HSU65533 Human regulator of | | |
| | | nonsense transcript stability (RENT1) mRNA, complete cds | | |
| 1201 | Z71367 | S. cerevisiae chromosome XIV reading frame ORF | 0.14 | |
| 1202 | J04809 | Human cytosolic adenylate kinase (AK1) gene, complete | 0.14 | |
| 1203 | X75014 | M. musculus Phox2 ruRNA for homeodomain protein | 0.14 | |
| 1204 | Y12259 | R. norvegicus mRNA for Kir3.1 protein | 0.14 | |
| 1205 | X81981 | L. helveticus plasmid pLH2 DNK (4 ORFs) | 0.14 | |
| 1206 | AB001901 | Homo sapiens PACE4 gene, exon 4-7 | 0.14 | |
| 1207 | D38548 | Human mRNA for KIAA0076 gene, complete cds | 0.14 | |
| 1208 | X64273 | Herpesvirus saimiri gene 15 > :: gb\|I41338\|I41338 Sequence | 0.13 | |
| | | from U.S. Pat. No. 5624837 > :: gb\|I49086\|I49086 Sequence | | |
| | | 9 from U.S. Pat. No. 5627264 | | |
| 1209 | Z92779 | Caenorhabditis elegans cosmid C44E1, complete sequence | 0.13 | |
| | | [Caenorhabditis elegans] | | |
| 1210 | X14384 | Astasia longa chioroplast rp15 gene for ribosomal protein L5 | 0.13 | |
| 1211 | M97880 | Bacillus thuringiensis cryIC-related gene sequence. > :: | 0.13 | |
| | | gb\|I82521\|I82521 Sequence 1 from U.S. Pat. No. 5712248 > :: | | |
| | | gb\|I93759\|I93759 Sequence 1 from U.S. Pat. No. 5731194 | | |
| 1212 | X58049 | H. sapiens DNA for ferredoxin repeat region | 0.13 | |
| 1213 | U47542 | Vibrio cholerae ADP-L-glycero-D-mannoheptose-6- | 0.13 | |
| | | epimerase (rfaD) gene, complete cds. | | |
| 1214 | M90758 | Sheep (clone C5-2) MHC class II cell surface glycoprotein | 0.13 | |
| | | OLA-DRB (OLA-DRB) pseudogene, exon 4. > :: | | |
| | | gb\|S83918\|S83914S3 DRB = MHC class II b {pseudogene} | | |
| 1215 | U67478 | Methanococcus jannaschii section 20 of 150 of the complete | 0.13 | |
| 1216 | U90889 | Mus musculus transketolase (TKT) gene, partial cds | 0.13 | |
| 1217 | K03273 | C. elegans heat shock protein genes (hsp16-48 and hsp16-1), | 0.13 | |
| | | complete cds. | | |
| 1218 | D90736 | Escherichia coli genomic DNA. (22.6 -23.0 min) | 0.13 | |
| 1219 | X63546 | H. sapiens mRNA for tre oncogene (clone 210) | 0.13 | |
| 1220 | L16770 | Anas platyrhynchos mitochondrial complete transfer RNA- | 0.13 | |
| | | Glu, transfer RNA-Phe, transfer RNA-Val, transfer RNA- | | |
| | | Leu, 12S ribosomal RNA, and 16S ribosomal RNA genes | | |
| 1221 | U94410 | Dictyostelium discoideum plasmid Ddp6 Rep protein | 0.13 | |
| 1222 | AC001083 | Homo sapiens (subclone 2_a6 from BAG H75) DNA | 0.13 | |
| 1223 | Z96048 | Caenorhabditis elegans cosmid F57A10, complete sequence | 0.13 | |
| | | [Caenorhabditis elegans] | | |
| 1224 | M92914 | Drosophila virilis mastermind gene, complete cds | 0.13 | |
| 1225 | Z82961 | Drosophila virilis mastermind gene, complete cds | 0.13 | |
| 1226 | U66535 | Human beta4-integrin (ITGB4) gene, exons | 0.13 | |
| 1227 | NM_001432.1 | Homo sapiens epiregulin (EREG) mRNA > :: | 0.13 | |
| | | dbj\|D30783\|D30783 Homo sapiens mRNA for epiregulin, | | |
| 1228 | X96468 | P. tetraurelia gamma1-51D immobilisation antigen gene, 3′ | 0.13 | |
| | | coding and non-coding region | | |
| 1229 | X05918 | Kluyveromyces fragilis beta-glucosidase gene | 0.13 | |
| 1230 | Z48243 | A. thaliana PARP mRNA for PARP protein | 0.13 | |
| 1231 | X56276 | Human Hut 2 End gene | 0.13 | |
| 1232 | X55318 | Mus musculus Hox-3.2 gene | 0.13 | |
| 1233 | U90009 | Phalacrocorax pelagicus cytochrome b gene, mitochondrial | 0.13 | |
| | | gene encoding mitochondrial protein, partial cds | | |
| 1234 | L76571 | Homo sapiens nuclear hormone receptor (shp) gene, 3′ end | 0.13 | |
| 1235 | U60581 | Human c-jun gene, promoter region with flanking | 0.13 | |
| | | evolutionary conserved sequences | | |
| 1236 | Z26886 | B. mori gene for Nd-sD mutant fibroin light chain | 0.13 | |
| 1237 | L19740 | Canis familiaris Kv1.2 delayed rectifier K+ channel mRNA, | 0.13 | |
| | | complete cds | | |
| 1238 | U26948 | Glycine max ribosomal protein S16 (rps 16) gene, partial cds, | 0.13 | |
| | | beta-carboxyltransferase (accD), photosystem I component | | |
| | | (psaI), ORE 202 protein (ORF 203), ORF 151 protein (ORF | | |
| | | 151), ORF 103 protein (ORF 103), ORF 229 precurso . . . | | |
| 1239 | U48288 | Rattits norvegicus A-kinase anchoring protein AKAP 220 | 0.13 | |
| | | mRNA, complete cds | | |
| 1240 | X70064 | P. deltoides gene for poplar bark storage protein | 0.13 | |
| 1241 | L22857 | Colletotrichum gloeosporioides pectin lyase (pn1A) gene, | 0.13 | |
| 1242 | X75607 | P. halophilus RepA and RepB genes | 0.13 | |
| 1243 | X82584 | M. musculus mRNA for immunoglobulin heavy chain, V | 0.13 | |
| 1244 | M76713 | Spodoptera frugiperda 16S rRNA gene, Val-tRNA, and Leu- | 0.13 | |
| | | tRNA genes, and ND-1 protein gene, 5′ end. | | |
| 1245 | X57075 | H. sapiens FGF6 gene | 0.13 | |
| 1246 | Z17201 | H. sapiens (DXS1003) DNA segment containing | 0.13 | |
| 1247 | D50931 | Human mRNA for KIAA0141 gene, complete cds | 0.13 | |
| 1248 | D80002 | Human mRNA for KIAA0180 gene, partial cds | 0.13 | |
| 1249 | M58600 | Human heparin cofactor II (HCF2) gene, exons 1 through 5. | 0.13 | |
| 1250 | Z79998 | Human DNA sequence from cosmid B5E3 on chromosome | 0.13 | |
| | | 22 Contains CpG island, EST | | |
| 1251 | X82868 | G. gallus microsatellite DNA (63G2) | 0.13 | |
| 1252 | AF009074 | Hepatitis C virus genomic RNA, 3′ nonstranslated region, | 0.13 | |
| | | partial sequence. clone #19 | | |
| 1253 | M26716 | Rat cAMP phosphodiesterase mRNA, complete cds., clone | 0.13 | |
| 1254 | Z57339 | H. sapiens CpG island DNA genomic Mse1 fragment, clone | 0.13 | |
| | | 171g3, reverse read cpg171g3.rt1a | | |
| 1255 | U76524 | Sambucus nigra ribosome inactivating protein precursor | 0.13 | |
| | | mRNA, complete cds | | |
| 1256 | AF000582 | Mus musculus nuclear receptor coactivator protein 2 mRNA, | 0.13 | |
| | | complete cds | | |
| 1257 | D12917 | Yeast gene for a component of the pheromone signal | 0.13 | |
| | | transduction pathway, complete cds | | |
| 1258 | X70326 | H. sapiens MacMarcks mRNA | 0.13 | |
| 1259 | X82237 | S. chinensis RAPD DNA (523 bp) | 0.13 | |
| 1260 | U10116 | Human superoxide dismutase (SOD3) gene, complete cds. | 0.13 | |
| 1261 | X73293 | M. vannielii genes rpoH, rpoB and rpoA | 0.13 | |
| 1262 | U01766 | Mycoplasma genitalium major adhesion protein MgPa gene, | 0.13 | |
| | | partial cds | | |
| 1263 | X79706 | C. aietinum capr1 mRNA for pathogenesis-related protein | 0.13 | |
| 1264 | U45957 | Nicotiana alata SA2-RNase precursor gene, complete cds. | 0.13 | |
| 1265 | X66313 | H. sapiens GLUDP2 gene (exon 2) | 0.13 | |
| 1266 | X07946 | Yeast plasmid DNA coding for RNA polymerase subunit | 0.13 | |
| 1267 | X07870 | Drosophila melanogaster bicoid gene bcd | 0.13 | |
| 1268 | X15308 | H. sapiens NF-H gene, exon 3 | 0.13 | |
| 1269 | Z22551 | H. sapiens kinectin gene | 0.13 | |
| 1270 | X89398 | H. sapiens ung gene for uracil DNA-glycosylase | 0.13 | |
| 1271 | D10654 | S. coelicolor afsQ1 and afsQ2 genes | 0.13 | |
| 1272 | U10516 | Human DNA polymerase beta gene, exons 1 and 2 | 0.13 | |
| 1273 | X57075 | H. sapiens FGF6 gene | 0.13 | |
| 1274 | U71193 | Mus musculus Rp123 matrix-attachment region | 0.13 | |
| 1275 | X69908 | H. sapiens gene for mitochondrial ATP synthase c subunit | 0.13 | |
| 1277 | X78401 | Bacteriophage P22 fight operon, orf 48, replication genes 18 | 0.13 | |
| | | and 12, nin region genes, ninG phosphatase, late control | | |
| | | gene 23, orf60, complete cds, late control region, start of | | |
| 1278 | X78401 | Bacteriophage P22 right operon, orf 48, replication genes 18 | 0.13 | |
| | | and 12, nin region genes, ninG phosphatase, late control | | |
| | | gene 23, orf 60, complete cds, late control region, start of | | |
| 1279 | Y09852 | H. sapiens FGFR3 gene, partial | 0.13 | |
| 1280 | Z71367 | S. cerevisiae chromosome XIV reading frame ORF | 0.13 | |
| 1281 | U38292 | Human microtubule-associated protein 1a(MAP1A) mRNA, | 0.13 | |
| | | complete cds | | |
| 1282 | Z71367 | S. cerevisiae chromosome XIV reading frame ORF | 0.13 | |
| 1283 | Z71367 | S. cerevisiae chromosome XIV reading frame ORF | 0.13 | |
| 1284 | M87623 | Babesia bovis variable antigen 1 mRNA 5′ end. | 0.13 | |
| 1285 | M91452 | Sus scrofa ryanodine receptor (RYR1) gene, complete cds. | 0.13 | |
| 1286 | M91452 | Sus scrofa ryanodine receptor (RYR1) gene, complete cds. | 0.13 | |
| 1287 | M87623 | Babesia bovis variable antigen 1 mRNA, 5′ end. | 0.13 | |
| 1288 | X96616 | P. primaurelia gene encoding 156D surface antigen | 0.13 | |
| 1289 | Z33222 | M. capricolum DNA for CONTIG MC303 | 0.12 | |
| 1290 | X52626 | Phaseolus vulgaris gene for alpha-phaseolin | 0.12 | |
| 1291 | L32699 | Paracentrotus lividus glutamine synthetase (Gs) mRNA, | 0.12 | |
| 1292 | M63669 | Dog arginine esterase gene, complete cds. | 0.12 | |
| 1293 | X75653 | A. longa plastid genes for ribosomal proteins and tRNAs | 0.12 | |
| 1294 | Z11839 | T. maritima nusG gene and genes for ribosomal proteins | 0.12 | |
| 1295 | AB005744 | Perilla frutescens DNA for 1-limonene synthase, complete | 0.12 | |
| 1296 | X84904 | Plasmodium falciparum encoding Pfg27/25 | 0.12 | |
| 1297 | L02074 | Drosophila melanogaster ribosomal protein S6 gene and two | 0.12 | |
| | | potential alternatively spliced proteins, complete cds | | |
| 1298 | Z84812 | Human DNA sequence from phage pTEL from a contig from | 0.12 | |
| | | the tip of the short arm of chromosome 16, spanning 2 Mb of | | |
| | | 16p13.13 Contains ESTs | | |
| 1299 | X95276 | P. falciparum complete gene map of plastid-like DNA | 0.12 | |
| 1300 | NM_001432.1 | Homo sapiens epiregulin (EREG) mRNA > :: | 0.12 | |
| | | dbj\|D30783\|D30783 | Homo sapiens mRNA for epiregulin, | |
| 1301 | X84726 | M. musculus neurocan gene | 0.12 | |
| 1302 | Z35810 | S. cerevisiae chromosome II reading frame ORF YBL049w | 0.12 | |
| 1303 | L01655 | Plasmodium falciparum T9/106 triosephosphate isomerase | 0.12 | |
| 1304 | U36796 | Mus musculus presynaptic protein SNAP-25 (Snap-25) gene, | 0.12 | |
| 1305 | U22361 | Saccharomyces cerevisiae Rlr1p (RLR1) gene, complete cds. | 0.12 | |
| 1306 | L04161 | Plasmodium falciparum (clone Pfg377 [PfsXLX]) DNA | 0.12 | |
| | | sequence, complete cds | | |
| 1307 | U32768 | Haemophilus influenzae Rd section 83 of 163 of the | 0.12 | |
| 1308 | D28808 | Mycoplasma capricolum mt1A and gyrB genes for DNA | 0.12 | |
| | | gyrase subunit B and mannitol-specific phosphotransferase | | |
| 1309 | L05920 | Human constitutively expressed serum amyloid A protein | 0.12 | |
| | | (SAA4) gene, exons 1 through 4. | | |
| 1310 | M96642 | Paramecium tetraurelia P126 repetitive element. | 0.12 | |
| 1311 | L81724 | Homo sapiens (subclone 2_a3 from P1 H69) DNA sequence | 0.12 | |
| 1312 | AF010331 | Tenebrio molitor thermal hysteresis protein isoform YL-3 | 0.12 | |
| | | precursor, mRNA, complete cds | | |
| 1313 | U74496 | Human chromosome 4q35 subtelomeric sequence | 0.12 | |
| 1314 | U17295 | Haemophilus influenzae dppB, dppC, dppD, dppF, isn, artP | 0.12 | |
| | | artI/J, artQ, and artM genes, complete cds, and opa gene, | | |
| 1315 | X53921 | A. thaliana T-DNA insertion genomic target sequence 621- | 0.12 | |
| 1316 | X75653 | A. longa plastid genes for ribosomal proteins and tRNAs | 0.12 | |
| 1317 | U09347 | Human variant urokinase plasminogen activator receptor | 0.12 | |
| | | (uPAR2) mRNA, partial cds. | | |
| 1318 | S83182 | hyaluronan-binding protein = hepatocyte growth factor | 0.12 | |
| | | activator homolog [human, plasma, mRNA, 2408 nt] | | |
| 1319 | U21164 | Human dopamine D5 receptor gene, 5′ flanking and | 0.12 | |
| 1320 | AF022391 | Feline herpesvirus 1 immediate early protein, glycoprotein | 0.12 | |
| | | gL, and uracil DNA glycosylase genes, complete cds | | |
| 1321 | M74569 | Clostridium acetobutylicum heat shock protein | 0.12 | |
| 1322 | X15359 | Drosophila virilis hunchback (hb) gene for zinc-finger | 0.12 | |
| | | protein transcription factor | | |
| 1323 | U67478 | Methanococcus jannaschii section 20 of 150 of the complete | 0.12 | |
| 1324 | X77052 | Entomopoxvirus gene for spherulin | 0.12 | |
| 1325 | M97514 | Saccharomyces douglasii mitochondrial cytochrome c | 0.12 | |
| | | oxidase subunit I (COXI) gene, complete cds | | |
| 1326 | X56495 | H. sapiens DNA for the upstream regulatory region of the c- | 0.12 | |
| | | erbB2 gene | | |
| 1327 | D61398 | Human MSH3 gene, exon3 | 0.12 | |
| 1328 | Z81595 | Caenorhabditis elegans cosmid T22H2, complete sequence | 0.12 | |
| | | [Caenorhabditis elegans] | | |
| 1329 | D61398 | Human MSH3 gene, exon3 | 0.12 | |
| 1330 | Z79998 | Human DNA sequence from cosmid B5E3 on chromosome | 0.12 | |
| | | 22 Contains CpG island, EST | | |
| 1331 | NM_001280.1 | Homo sapiens cold inducible RNA-binding protein (CIRBP) | 0.12 | |
| | | mRNA > :: dbj\|D78134\|HUMCIRPA Homo sapiens mRNA | | |
| 1332 | Z49867 | Caenorhabditis elegans cosmid C33D3, complete sequence | 0.12 | |
| | | [Caenorhabditis elegans] | | |
| 1333 | X14036 | Tomato cab-7 gene for type II chlorophyll a/b-bin | 0.12 | |
| 1334 | AF012899 | Sambucus nigra ribosome inactivating protein precursor | 0.12 | |
| | | mRNA, complete cds | | |
| 1335 | U19159 | Human P protein (P) gene, exon 8 | 0.12 | |
| 1336 | X74987 | H. sapiens mRNA for 2′-5′ oligoadenylate binding protein > | 0.12 | |
| | | :: gb\|L24115\|HUMAAZ Human DNA fragment. > :: | | |
| | | emb\|A49723.1\|A49723 Sequence 2 from Patent | | |
| 1337 | U32112 | Dictyostelium discoideum 34 kDa actin binding protein | 0.12 | |
| | | gene, complete cds > :: emb\|Z50156\|DD30KDABP | | |
| | | D. discoideum gene for 34 kD actin binding protein | | |
| 1338 | M55002 | Synechococcus sp. 6-phosphogluconate dehydrogenase | 0.12 | |
| | | gene, complete cds. | | |
| 1339 | AF019225 | Homo sapiens apolipoprotein L mRNA, complete cds | 0.12 | |
| 1340 | U14190 | Mycobacterium smegmatis diptheria toxin repressor | 0.12 | |
| | | homolog (dtxR) gene, complete cds. | | |
| 1341 | J05276 | Rat 5-hydroxytryptamine-1a receptor (5-HT-1a) gene, | 0.12 | |
| 1342 | AE000603.1 | Helicobacter pylon 26695 section 81 of 134 of the complete | 0.12 | |
| 1343 | X15308 | H. sapiens NF-H gene, exon 3 | 0.12 | |
| 1344 | L35600 | Homo sapiens DNA sequence. | 0.12 | |
| 1345 | Z73360 | Human DNA sequence from cosmid 92M18, BRCA2 gene | 0.12 | |
| | | region chromosome 13q12-13 | | |
| 1346 | U93721 | Homo sapiens green cone photoreceptor pigment gene, 5′ | 0.12 | |
| | | flanking region | | |
| 1347 | X78401 | Bacteriophage P22 tight operon, orf 48, replication genes 18 | 0.12 | |
| | | and 12, nin region genes, ninG phosphatase, late control | | |
| | | gene 23, orf 60, complete cds, late control region, start of | | |
| 1348 | Z71367 | S. cerevisiae chromosome XIV reading frame ORF | 0.12 | |
| 1349 | U43673 | Mus musculus putative transmembrane receptor IL-1Rrp | 0.12 | |
| | | mRNA, complete cds > :: gb\|AR016448\|AR016448 | | |
| | | Sequence 3 from U.S. Pat. No. 5776731 | | |
| 1350 | X72863 | A. thaliana TMKL1 mRNA | 0.12 | |
| 1351 | U13769 | Vibrio sp. ppGpp synthetase I (relA) gene, complete cds. | 0.12 | |
| 1352 | M32732 | Chicken progesterone receptor gene, encoding forms A and | 0.11 | |
| 1353 | X04668 | Xenopus laevis XK81A1 keratin gene > :: | | |
| | | emb\|X04804\|XLXK81A1 Xenopus laevis DNA for stage- | | |
| | | specific epidermal type I keratin A1 (embryo- and larval- | | |
| 1354 | Z82197 | Human DNA sequence from PAC 293L6 on chromosome | 0.11 | |
| | | 22, complete sequence [Homo sapiens] | | |
| 1355 | M84214 | Cucumis sativus ORF 1, chintinase, and ORF 3 genes, | 0.11 | |
| | | complete cds > :: gb\|I38466\|I38466 Sequence 36 from U.S Pat. | | |
| | | No. 5614395 > :: gb\|I56941 \|I56941 Sequence 36 from U.S. Pat. | | |
| | | No. 5650505 > :: gb\|I59807\|I59807 Sequence 36 from U.S. Pat. | | |
| | | No. 5654414 > :: gb\|I75134\|I7513 | | |
| 1356 | Y08503 | F. domesticus mitochondrial 12S rRNA gene | 0.11 | |
| 1357 | L03286 | Hamster P-glycoprotein class I (pgp1) gene exons 1-2. | 0.11 | |
| 1358 | X57171 | D. caryophyllus CARSR12 gene | 0.11 | |
| 1359 | L07307 | Mus musculus ATPase mRNA-ampifying genomic DNA | 0.11 | |
| | | MOR6.5 sequence. > :: gb\|S55685\|S55685 | | |
| | | MOR6.5 = ouabain resistance gene {repeat sequence} [mice, | | |
| 1360 | Z22175 | Caenorhabditis elegans cosmid K01F9, complete sequence | 0.1 | |
| | | [Caenorhabditis elegans] | | |
| 1361 | Z11839 | T. maritima nusG gene and genes for ribosomal proteins | 0.1 | |
| 1362 | L36857 | Pisum sativum GTP-binding protein (IAP86) mRNA, | 0.096 | |
| 1363 | AF000994 | Homo sapiens ubiquitous TPR motif, Y isoform | 0.07 | |
| 1364 | U61950 | Caenorhabditis elegans cosmid C45E5 | 0.069 | |
| 1365 | U32446 | Mus musculus putative breast/ovarian cancer susceptibility | 0.068 | |
| | | protein homolog (Brca1) mRNA, complete cds. | | |
| 1366 | AB001914 | Homo sapiens PACE4 gene, exon 23-25, complete cds | 0.068 | |
| 1367 | L20934 | Anopheles gambiae complete mitochondrial genome | 0.066 | |
| 1368 | AJ001700 | Mus musculus mRNA for neuroserpin | 0.065 | |
| 1369 | AJ001700 | Mus musculus mRNA for neuroserpin | 0.065 | |
| 1370 | AF106932 | Drosophila melanogaster plexin A (plexA) mRNA, complete | 0.065 | |
| 1371 | D82813 | Populus kitakamiensis cyp73b gene for cinnamic acid 4- | 0.065 | |
| | | hydroxylase, partial cds | | |
| 1372 | V01087 | Hemagglutinin gene of influenza virus strain | 0.065 | |
| 1373 | AE001178 | Borrelia burgdorferi (section 64 of 70) of the complete | 0.064 | |
| 1374 | X80199 | H. sapiens MLN51 mRNA | 0.064 | |
| 1375 | AF042384 | Homo sapiens BC-2 protein mRNA, complete cds | 0.064 | |
| 1376 | AJ223485 | Enoploteuthis higginsi mitochondrial 16S rRNA | 0.063 | |
| 1377 | AF086094 | Homo sapiens full length insert cDNA clone YZ87H06 | 0.061 | |
| 1378 | AF017027 | African swine fever virus lectin homolog (8CR) gene, | 0.061 | |
| 1379 | U56084 | Bordetella bronchiseptica electron transfer flavoprotein | 0.06 | |
| | | alpha subunit (etfA) gene, partial cds, and exogenous ferric | | |
| | | sideerophore receptor (bfrA) gene, complete cds | | |
| 1380 | M33387 | Human debrisoquine 4-hydroxylase (CYP2D8P) and | 0.057 | |
| 1381 | M25315 | Homo sapiens (clone pAT 464) potential | 0.057 | |
| | | lymphokine/cytokine mRNA, complete cds. | | |
| 1382 | AB011121 | Homo sapiens mRNA for KIAA0549 protein, partial cds | 0.055 | |
| 1383 | NM_002647.1 | Homo sapiens phosphatidylinositol 3-kinase, class 3 | 0.051 | |
| | | (PIK3C3) mRNA > :: emb\|Z46973\|HSPITR1 H. sapiens | | |
| | | mRNA for phosphatidylinositol 3-kinase | | |
| 1384 | Z18274 | Homo sapiens satellite DNA | 0.05 | |
| 1385 | L25941 | Homo sapiens integral nuclear envelope inner membrane | 0.049 | |
| | | protein (LBR) gene, complete cds. | | |
| 1386 | U28171 | Trypanosoma cruzi kinetoplast putative maxicircle divergent | 0.049 | |
| 1387 | X76168 | R. norvegicus mRNA for connexin 30.3 | | |
| 1388 | L34345 | Morganella morganii xylitol repressor, complete cds; xylitol | 0.049 | |
| | | dehydrogenase, complete cds; xylulokinase, 5′ end. | | |
| 1389 | K01322 | Human Ig germline kappa L-chain V-region gene germline | 0.049 | |
| | | kappa L-chain V-region gene (HK189), V-kappa-1. | | |
| 1390 | S72468 | capsid protein {RNA} [tomato aspermy virus TAV-B, | 0.049 | |
| | | Blencowe, Genomic RNA, 2213 nt] | | |
| 1391 | L08426 | Zea mays auxin-binding protein (abp4) gene, exons 1-5 and | 0.048 | |
| | | complete cds. | | |
| 1392 | X96972 | D. buzzatii copia element DNA | 0.048 | |
| 1393 | X17147 | Canine mRNA for thyrotropin (TSH) receptor variant | 0.048 | |
| 1394 | L13164 | Saccharomyces cerevisiae k9 killer toxin resistant | 0.047 | |
| 1395 | X66451 | E. octocarinatus RPA2 gene for RNA polymerase I second | 0.047 | |
| | | largest subunit | | |
| 1396 | X75426 | G. biloba (developing endosperm) ginnacin mRNA | 0.047 | |
| 1397 | M95171 | Aedes aegypti LINE Juan-A including DNA | 0.047 | |
| | | binding protein and reverse transcriptase-like protein | | |
| | | mRNA, complete coding regions. | | |
| 1398 | Z49076 | R. prowazekii gene (unknown) | 0.047 | |
| 1399 | K01323 | Human Ig germline kappa L-chain V-region gene germline | 0.047 | |
| | | immunoglobulin heavy chain, kappa chain, | | |
| 1400 | L48713 | Homo sapiens galactose-1-phosphate uridyl transferase | 0.047 | |
| | | (GALT) mutant V44L gene, exon 7 (M96246 bases 303- | | |
| 1401 | U77310 | Drosophila melanogaster porcupine mRNA, complete cds | 0.047 | |
| 1402 | J01323 | Yeast (S. cerevisiae) enolase gene (clone peno8) and flanks. | 0.047 | |
| 1403 | L14321 | Bovine herpesvirus type 1 immedidate-early transcriptional | 0.047 | |
| | | control protein (BICP4) gene, 5′ end. | | |
| 1404 | L19266 | Homo sapiens myotonic dystrophy-associated protein kinase | 0.047 | |
| | | and 59 genes. | | |
| 1405 | M58600 | Human heparin cofactor II (HCF2) gene, exons 1 through 5. | 0.046 | |
| 1406 | D86964 | Human mRNA for KIAA0209 gene, partial cds | 0.046 | |
| 1408 | L27331 | Glyphinaphis bambusae mitochondrial cytochrome oxidase | 0.046 | |
| | | subunit I gene, 3′ end, and cytochrome oxidase subunit II | | |
| 1409 | U57613 | Human interleukin-2 receptor alpha chain (IL2RA) gene, | 0.046 | |
| | | promoter region | | |
| 1410 | U24088 | Solanum tuberosum sucrose synthase clone gPOSS65, | 0.046 | |
| | | complete cds. | | |
| 1411 | V01087 | Hemagglutinin gene of influenza virus strain | 0.046 | |
| 1412 | S76792 | OX40 = cell surface antigen [human, mRNA Partial, 1034 nt] | 0.046 | |
| 1413 | U72396 | Lycopersicon esculentum class II small heat shock protein | 0.046 | |
| | | Le-HSP17.6 mRNA, complete cds | | |
| 1414 | U51677 | Human non-histone chromatin protein HMG1 (HMG1) gene, | 0.046 | |
| | | complete cds | | |
| 1415 | X98743 | H. sapiens mRNA for RNA helicase (Myc-regulated dead | 0.046 | |
| 1416 | M63868 | C. hircus alpha-lactalbumin gene, exons 1-4. | 0.046 | |
| 1417 | Z92970 | Caenorhabditis elegans cosmid H06O01, complete sequence | 0.045 | |
| | | [Caenorhabditis elegans] | | |
| 1418 | M29482 | Human leukocyte adhesion protein p150, 95 alpha subunit | 0.045 | |
| | | gene, exons 2-6. | | |
| 1419 | Z78942 | H. sapeins flow-sorted chromosome 6 HindIII fragment, | 0.045 | |
| 1420 | L06863 | Cricetulus griseus collagen mRNA, 3′ end. | 0.045 | |
| 1421 | X86449 | E. caballus DNA segment containing microsatellite | 0.045 | |
| 1422 | U32446 | Mus musculus putative breast/ovarian cancer susceptibility | 0.045 | |
| | | protein homolog (Brca1) mRNA, complete cds. | | |
| 1423 | K01323 | Human Ig germime kappa L-chain V-region gene germline | 0.045 | |
| | | immunoglobulin heavy chain, kappa chain, | | |
| 1424 | M13555 | Human Ia-associated invariant gamma-chain gene, exon 1 | 0.045 | |
| | | clones lambda-y(1, 2, 3). | | |
| 1425 | U68562 | Rattus norvegicus chaperonin 60 (Hsp60) and chaperonin 10 | 0.045 | |
| | | (CPN10) genes, nuclear genes encoding mitochondrial | | |
| 1426 | AB003286 | Homo sapiens DNA for choline kinase like protein and | 0.045 | |
| | | muscle type carnitine palmitoyltransferase I, partial and | | |
| 1427 | U34372 | Human tyrosine kinase TXK (txk) gene, exon 6. | 0.045 | |
| 1428 | U51677 | Human non-histone chromatin protein HMG1 (HMG1) gene, | 0.045 | |
| | | complete cds | | |
| 1429 | U23476 | Dictyostelium discoideum phosphatidylinositol-4,5- | 0.045 | |
| | | diphosphate 3-kinase (PIK1) mRNA, complete cds. | | |
| 1430 | Z98975 | S. pombe chromosome I cosmid c19E9 | 0.044 | |
| 1431 | X16465 | Trypanosoma brucei mRNA for cysteine proteinase | 0.044 | |
| 1432 | D85274 | Macaca fascicularis mitochondrial DNA for NADH | 0.044 | |
| | | dehydrogenase subunit 4, subunit 5, partial cds | | |
| 1433 | X16876 | Soybean ENOD2B gene for Ngm-75 | 0.044 | |
| 1434 | U19755 | Mus domesticus thyroid transcription factor 1 gene, | 0.044 | |
| 1435 | L77700 | Gallus gallus 18C15 mRNA, complete cds. | 0.044 | |
| 1436 | AF019981 | Dictyostelium discoideum HelE (helE) gene, partial cds | 0.044 | |
| 1437 | L13469 | Saccharomyces cerevisiae antivirual protein Ski2p | 0.044 | |
| 1438 | M26238 | D. discoideum spore coat protein SP70 gene, complete cds. | 0.044 | |
| 1439 | U65391 | Lycoperisicon esculentum PRF (Prf) gene, complete cds | 0.044 | |
| 1440 | AF000582 | Mus musculus nuclear receptor coactivator protein 2 mRNA, | 0.044 | |
| | | complete cds | | |
| 1441 | X98880 | C. albicans ARG5, 6 gene | 0.044 | |
| 1442 | D89609 | Oryzias latipes mRNA for choriogenin H, complete cds | 0.044 | |
| 1443 | Y13544 | Homo sapiens cosmid C1 | 0.044 | |
| 1444 | Y14952 | Mus musculus gene encoding immunoglobulin J chain | 0.043 | |
| 1445 | Z69660 | Caenorhabditis elegans cosmid F39B1, complete sequence | 0.043 | |
| | | [Caenorhabditis elegans] | | |
| 1446 | X53404 | Glycine max glycinin A(1a)B(1b) and A(2)B(1a) boundary | 0.043 | |
| 1447 | U48271 | Dictyostelium discoideum UbpA deubiquitinase mRNA, | 0.043 | |
| 1448 | U24187 | Saccharomyces cerevisiae origin recognition complex, | 0.043 | |
| | | subunit 5 (ORC5) gene, complete cds > :: gb\|I32734\|I32734 | | |
| | | Sequence 9 from U.S. Pat. No. 5589341 > :: gb\|I38710\|I38710 | | |
| | | Sequence 9 from U.S. Pat. No. 5614618 | | |
| 1449 | Z28177 | S. cerevisiae chromosome XI reading frame ORF YKL178c | 0.043 | |
| 1450 | X05951 | Drosophila melanogaster calmodulin gene exon 4 and intron | 0.043 | |
| | | III (partial) | | |
| 1451 | L12999 | Daltonia sp. mitochondrial 16S ribosomal RNA | 0.043 | |
| 1452 | U68098 | Human poly(A)-binding protein (PABP) gene, exons 6 and 7 | 0.043 | |
| 1453 | US5774 | Caenorhabditis elegans cosmid F35G8. | 0.043 | |
| 1454 | Z46941 | H. sapiens DNA for alu repeats | 0.043 | |
| 1455 | U41747 | Caenorhabditis elegans cosmid ZK1131. | 0.043 | |
| 1456 | M30168 | D. melanogaster repetitive sequences F and G, | 0.043 | |
| 1457 | U05350 | Human immunodeficiency virus type 2 isolate HIV2CBL21 | 0.042 | |
| | | gp160 envelope (env) gene, complete cds. | | |
| 1458 | U44129 | Rattus norvegicus p58 mRNA, complete cds | 0.042 | |
| 1459 | L13377 | Staphylococcus aureus enterotoxin gene, 3′ end. | 0.042 | |
| 1460 | X83758 | P. falciparum topoisomerase I gene | 0.042 | |
| 1461 | U64896 | Anagrapha falcifera nuclear polyhedrosis virus protein- | 0.042 | |
| | | typrosine phosphatase, lef-2, polyhedrin, vp78 and protein | | |
| | | kinase genes, complete cds | | |
| 1462 | J02175 | Influenza A/wsn/33 (h1n1). defective interfering 13, cdna. | 0.042 | |
| 1463 | D86608 | Arabidopsis thaliana DNA for inorganic phosphate | 0.042 | |
| | | transporter, complete cds | | |
| 1464 | M93716 | Pan paniscus DNA fragment. | 0.042 | |
| 1465 | X14385 | Astasia longa chloroplast rps7 and tufA genes for ribosomal | 0.042 | |
| | | protein S7 and elongation factor Tu respectively | | |
| 1466 | M74069 | Saccharomyces cerevisiae endochitinase (CTS1-1) gene, | 0.042 | |
| | | complete cds. | | |
| 1467 | M73257 | C. parasitica laccase (lac-1) gene, complete cds. | 0.042 | |
| 1468 | M31126 | Human pregnancy-specific bet-1-glycoprotein (SP1) | 0.042 | |
| | | mRNA, complete cds, clone hPS2. | | |
| 1469 | U70826 | Fundulus heteroclitus vitellogenin II precursor mRNA, | 0.041 | |
| 1470 | AF000299 | Caenorhabditis elegans cosmid E03H12 | 0.041 | |
| 1471 | X83390 | Albinaria coerulea complete mithochondria DNA | 0.041 | |
| 1472 | L12582 | Human ornithine decarboxylase (ODC) gene, 5′ flanking | 0.041 | |
| 1473 | D89655 | Rattus norvegicus mRNA for scavenger receptor class B | 0.041 | |
| | | complete cds | | |
| 1474 | Z23267 | P. anserina AS1 gene, complete CDS | 0.041 | |
| 1475 | M63868 | C. hircus alpha-lactalbumin gene, exons 1-4. | 0.041 | |
| 1476 | U56440 | Human His-1 gene sequence | 0.04 | |
| 1477 | L06863 | Cricetulus griseus type VII collagen mRNA, 3′ end. | 0.04 | |
| 1478 | U02928 | Dictyostelium discoideum Ax3 Rab7 mRNA, complete cds. | 0.04 | |
| 1479 | M19828 | Human apolipoprotein B-100 (apoB) gene, exons 22 through | 0.04 | |
| 1480 | M34434 | Human angiotensin-converting enzyme (ACE) gene, 5′ end. | 0.04 | |
| 1481 | Z68884 | Human DNA sequence from cosmid L21F12, Huntington's | 0.04 | |
| | | Disease Region, chromosome 4p16.3 | | |
| 1482 | M99367 | Pig alveolar macrophage-derived chemotactic factor-I | 0.04 | |
| | | (AMCF-I) mRNA, compete cds. | | |
| 1483 | U70370 | Human hindlimb expressed homeobox protein backfoot (Bft) | 0.04 | |
| | | mRNA, complete cds | | |
| 1484 | U10698 | Rattus norvegicus liver microsomal carboxylesterase mRNA, | 0.04 | |
| | | complete cds. | | |
| 1485 | U28171 | Trypanosoma cruzi kinetoplast putative maxicircle divergent | 0.04 | |
| 1486 | U79297 | Human clone 23589 mRNA sequence | 0.04 | |
| 1487 | X13764 | Yeast centromere CEN1 repetitive DNA PSS113, part of | 0.04 | |
| 1488 | Z24568 | H. sapiens (D9S286) DNA segment containing | 0.04 | |
| 1489 | M63868 | C. hircus alpha:lactalbumin gene, exons 1-4. | 0.04 | |
| 1490 | X64143 | F. trinervia ppcA1 gene for phosphoenolpyruvate | 0.039 | |
| 1491 | M16339 | Entamoeba histolytica actin mRNA, complete cds. | 0.039 | |
| 1492 | U36475 | Mus musculus breast and ovarian cancer susceptibility | 0.039 | |
| | | protein (Brca1) mRNA, complete cds | | |
| 1493 | X02570 | Flesh fly gene for 25-kDa protein | 0.039 | |
| 1494 | M32475 | Rattus norvegicus carcinoembryonic antigen-related protein | 0.039 | |
| | | (CGM4) gene, exons 2 and 3 | | |
| 1495 | D89609 | Oryzias latipes mRNA for choriogenin H, complete cds | 0.039 | |
| 1496 | AF011573 | Homo sapiens finger protein (ZnF20) mRNA, complete | 0.039 | |
| 1497 | X15050 | Mouse mRNA for 3′-end of NCAM-120 isoform | 0.039 | |
| 1498 | Z11533 | D. discoideum rasG gene | 0.038 | |
| 1499 | AB000450 | Homo sapiens mRNA for VRK2, complete cds | 0.038 | |
| 1500 | X78983 | D. melanogaster Fab-7 regulatory region | 0.038 | |
| 1501 | D89609/ Oryzias latipes mRNA for choriogenin H, complete cds | 0.038 | | |
| 1502 | Z49535 | S. cerevisiae chromosome X reading frame ORF YJR035w | 0.038 | |
| 1503 | L13377 | Staphylococcus aureus enterotoxin gene, 3′ end. | 0.035 | |
| 1504 | L12582 | Human ornithine decarboxylase (ODC) gene, 5′ flanking | 0.035 | |
| 1505 | Z48584 | Caenorhabditis elegans cosmid ZK1321, complete sequence | 0.023 | |
| | | [Caenorhabditis elegans ] | | |
| 1506 | Y00204 | Xenopus laevis mRNA fragment for nucleoplasmin | 0.021 | |
| 1507 | S77817 | light-chain fibroin [Galleria mellonell = waxmoths, larvae, | 0.021 | |
| | | cocoons, posterior silk glands, mRNA, 1191 nt] | | |
| 1508 | L13926 | Trypanosoma cruzi (strain La Cruz, Jalisco) ribosomal RNA | 0.02 | |
| | | transcribed spacer and 18S ribosomal RNA gene, 5′ end. | | |
| 1509 | Z78910 | H. sapiens flow-sorted chromosome 6 HindIII fragment, | 0.02 | |
| 1510 | AB007976 | Homo sapiens mRNA, chromosome 1 specific transcript | 0.02 | |
| 1511 | U01307 | Human scRNA (BC200 beta) psuedogene. | 0.017 | |
| 1512 | J03998 | P. falciparum glutamic acid-rich protein gnen, complete cds. | 0.017 | |
| 1513 | X93497 | H. sapiens TRAP gene, intron 4 (partial) | 0.017 | |
| 1514 | M15656 | Human aldolase B (ALDOB) gene, exons 7 through 9. | 0.016 | |
| 1515 | X74965 | H. sapiens ACPP gene for prostatic acid phosphatase (non- | 0.016 | |
| | | coding region) | | |
| 1516 | X75653 | A. longa piastid genes for ribosomal proteins and tRNAs | 0.016 | |
| 1517 | X75653 | A. longa plastid genes for ribosomal proteins and tRNAs | 0.016 | |
| 1518 | X58852 | Human MLC1emb gene for embryonic myosin alkaline light | 0.016 | |
| | | chain, exon 2 | | |
| 1519 | U69695 | Mus musculus hyaluronan synthase homolog mRNA, | 0.016 | |
| 1520 | S45332 | erythropoietin receptor [human, placental, Genornic, 8647 | 0.016 | |
| 1521 | U09865 | Alcaligenes eutrophus pyruvate dehydrogenase | 0.016 | |
| | | dihydrolipoamide dehydrogenase (pdhL), and ORF3 genes, | 0.016 | |
| 1522 | U67815 | Human primary Alu transcript | 0.016 | |
| 1523 | AE000466 | Escherichia coli K-12 MG1655 section 356 of 400 of the | 0.016 | |
| | | complete genome | | |
| 1524 | M80785 | M. musculus tissue factor promoter (Cf-3) gene, exon 1. | 0.016 | |
| 1525 | L37035 | Drosophila virilis brown protein (bw) gene, complete cds. | 0.016 | |
| 1526 | M15009 | Mouse steroid 21-hydroxylase A (21-OHase A) gene, | 0.016 | |
| 1527 | U67500 | Methanococcus jannaschii section 42 of 150 of the complete | 0.016 | |
| 1528 | AB000044 | Rhizoctonia solani 5.8S rRNA gene, complete sequence | 0.016 | |
| 1529 | X52956 | Human CAMII-psi3 calmodulin retropseudogene | 0.016 | |
| 1530 | U80581 | Pleurodeles waltl Wnt-7a mRNA, complete cds | 0.016 | |
| 1531 | Z69918 | Human DNA sequence from cosmid 91K3, Huntington's | 0.016 | |
| | | Disease Region, chromosome 4p16.3 contains CpG island | | |
| 1532 | Z98031 | Human immunodeficiency virus type 1 nef gene | 0.016 | |
| | | immunodeficiency virus type 1 nef gene (strain KU15-1) | | |
| 1533 | D45858 | Mus musculus mRNA for synaptotagumin III, complete cds | 0.016 | |
| 1534 | L24549 | Gallus gallus Gi2 protein alpha-subunit mRNA, complete | 0.016 | |
| 1535 | U66291 | Catagonus wagneri cytochrome b gene, mitochondrial gene | 0.016 | |
| | | encoding mitochondrial protein, partial cds | | |
| 1536 | X63436 | B. taurus mRNA for poly(A) polymerase | 0.016 | |
| 1537 | L23498 | Bovine microsatellite repeats | 0.015 | |
| 1538 | AF003086 | Plasmodium falciparum transcription factor homolog | 0.015 | |
| | | PfSNF2L mRNA, complete cds | | |
| 1539 | U17377 | Strongylocentrotus purpiratus cortical granule protein with | 0.015 | |
| | | LDL-receptor-like repeats mRINA, partial cds. | | |
| 1540 | L23498 | Bovine microsatellite repeats | 0.015 | |
| 1541 | X85117 | H. sapiens epb72 gene exons 2, 3, 4, 5, 6, 7 | 0.015 | |
| 1542 | Z16906 | H. sapiens (D14S73) DNA segment containing | 0.015 | |
| 1543 | X99400 | S. pneumoniae dacA gene and ORF | 0.015 | |
| 1544 | M32061 | Rat alpha-2B-adrenergic receptor (RNG-alpha-2) mRNA, | 0.015 | |
| 1545 | X80930 | S. cerevisiae RHC18 genes | 0.015 | |
| 1546 | U09865 | Alcaligenes eutrophus pyruvate dehydrogenase | 0.015 | |
| | | dihydrolipoamide dehydrogenase (pdhL), and ORF3 genes, | | |
| 1547 | Z22952 | Mus musculus BALB/c or p65 gene encoding p65 subunit of | 0.015 | |
| | | transcription factor NF-kappaB | | |
| 1548 | L34610 | Mus musculus parathyroid hormone/parathyroid hormone | 0.015 | |
| | | related-peptide receptor (PTHR) gene, exons 5-9. | 0.015 | |
| 1549 | Z54850 | H.sapiens CpG island DNA genomic Mse1 fragment, clone | 0.015 | |
| | | 169b5, reverse read cpg169b5.rt1a | | |
| 1550 | U01307 | Human scRNA (BC200 beta) pseudogene. | 0.015 | |
| 1551 | M54994 | Human bile salt-activated lipase (BAL) mRNA, complete | 0.015 | |
| 1552 | Z98031 | Human immunodeficiency virus type 1 nef gene | 0.015 | |
| | | immunodeficiency virus type 1 nef gene (strain KU15-1) | | |
| 1553 | Z21858 | M. Musculus mRNA for P domain protein | 0.015 | |
| 1554 | M33518 | Human HLA-B-associated transcript 2 (BAT2) gene, 3′ end. | 0.015 | |
| 1555 | AB001383 | Rattus norvegicus mRNA for sialoprotem, complete cds | 0.015 | |
| 1556 | U72787 | Homo sapiens cosmid clone U163C11 from Xp22.1-22.2, | 0.014 | |
| | | complete sequence [Homo sapiens] | | |
| 1557 | AF012899 | Sambucus nigra ribosome inactivating protein precursor | 0.014 | |
| | | mRNA, complete cds | | |
| 1558 | J03764 | Human, plasminogen activator inliibitor-1 gene, exons 2 to | 0.014 | |
| 1559 | AF012899 | Sambucus nigra ribosome inactivating protein precursor | 0.014 | |
| | | mRNA, complete cds | | |
| 1560 | AB003097 | Fruitfly strain g20 mitochondrial DNA, A+T-rich region, | 0.014 | |
| | | partial sequence | | |
| 1561 | AC001017 | Homo sapiens (subclone 2_g8 from P1 H43) DNA sequence | 0.014 | |
| 1562 | Z48484 | H. sapiens gene for tissue-type plasminogen activator | 0.014 | |
| 1563 | AC001460 | Homo sapiens (subclone 2_f4 from BAG H107) DNA | 0.014 | |
| 1564 | U09865 | Alcaligenes eutrophus pyruvate dehydrogenase | 0.014 | |
| | | dihydrolipoamide dehydrogenase (pdhL), and ORF3 genes, | | |
| 1565 | U00691 | Dictyostelium discoideum plasmid Ddp1 D2 orf, D1/D3 orf | 0.014 | |
| | | G4/D5 orf G5/D6 orf, G1 orf, G2/G3/D4 orf complete cds | | |
| 1566 | D16482 | Sarcophage peregrina mRNA for poly(ADP-ribose) | 0.014 | |
| 1567 | AF012899 | Sambucus nigra ribosome inactivating protein precursor | 0.014 | |
| | | mRNA, complete cds | | |
| 1568 | M63599 | Human myelin basic protein (MBP) gene, exon 1. | 0.014 | |
| 1569 | U03891 | Human phorbolin I mRNA, partial cds. | 0.014 | |
| 1570 | X53422 | D. grimshawi s18, s15, s19 and s16 chorion protein genes | 0.013 | |
| 1571 | X01870 | Yeast mitochondrial ori(o) repeat unit of petite mutant 4 | 0.013 | |
| | | (petite strain a-10/3/2/B11) | | |
| 1572 | Z92804 | Caenorhabditis elegans cosmid K05D4, complete sequence | 0.013 | |
| | | [Caenorhabditis elegans] | | |
| 1573 | M76377 | Human cysteine-rich protein (CRP) gene, exons 3 and 4. | 0.013 | |
| 1574 | FD29801 | Mouse mRNA for unknown product, complete cds | 0.013 | |
| 1575 | U77984 | Sus scrofa microsatellite S0058 sequence | 0.013 | |
| 1576 | Z55882 | H. sapiens CpG island DNA genomic Mse1 fragment, clone | 0.013 | |
| | | 70g8, reverse read cpg70g8.rt1a | | |
| 1577 | V00531 | Human interferon genes LeIF-L and LeIF-J and pseudogene | 0.013 | |
| | | LeIF-M with intergenic regions. These genes are located on | | |
| 1578 | Z74361 | S. cerevisiae chromosome IV reading frame ORF YDR065w | 0.013 | |
| 1579 | D00374 | Herpes simplex virus type 1 (HSV-1) genome, rightmost part | 0.013 | |
| | | of the long unique region (UL) and all of the internal long | | |
| 1580 | U53502 | Arabidopsis thaliana chromosome I cosmid g17311 DNA. | 0.013 | |
| 1581 | K03196 | Human interferon-beta-3 gene. | 0.013 | |
| 1582 | M23456 | Zea mays (clone pCIB808) encoding maize nitrite reductase | 0.013 | |
| | | (NiR) cDNA to mRNA, partial cds. | | |
| 1583 | X66120 | H. sapiens F8VWFL DNA of the von Willebrand factor | 0.013 | |
| | | pseudogene (5′portion) | | |
| 1584 | M63962 | Human gastric H,K-ATPase catalytic subunit gene, complete. | 0.011 | |
| 1585 | AF039592 | Homo sapiens succinate dehydrogenase subunit C | 0.007 | |
| 1586 | Y10908 | B. cereus cysA and wapA genes | 0.007 | |
| 1587 | AF048988 | Homo sapiens MutS homolog 5 (MSH5) gene, exons 1 | 0.007 | |
| 1588 | M048988 | Homo sapiens MutS homolog 5 (MSH5) gene, exons 1 | 0.007 | |
| 1589 | D90822 | E. coli genomic DNA, Kohara clone #331(40.1-40.4 min.) | 0.007 | |
| 1590 | D87903 | Mouse mRNA for ARF6, complete cds | 0.006 | |
| 1591 | L38851 | Mycobacterium tuberculosis cell surface protein | 0.006 | |
| 1592 | U66362 | Human neuronal nitric oxide synthase (nNOS) gene, | 0.005 | |
| | | alternatively spliced 5′ exon Tex 2 and flanking sequences | | |
| 1593 | L11670 | Human trausmembrane glycoprotein (CD53) gene, exons 2 | 0.005 | |
| 1594 | U20587 | Sus scrofa domestica sister of P-glycoprotein | 0.005 | |
| 1595 | U53016 | Human DXYS154 microsatellite sequence | 0.005 | |
| 1596 | Z29641 | Zea mays of USE gene encoding U3snRNA | 0.005 | |
| 1597 | D16473 | Human mRNA, Xq terminal portion | 0.005 | |
| 1598 | X02175 | Schizosaccharomyces pombe cdc10 start gene | 0.005 | |
| 1599 | U25029 | Human glucocorticoid receptor alpha mRNA, variant 3′ | 0.005 | |
| 1600 | Z68685 | Human DNA sequence from cosmid N2E9 on chromosome | 0.005 | |
| | | 22, complete sequence [Homo sapiensp] | | |
| 1601 | U40369 | Human spermidine/spermine N1-acetyltransferase | 0.005 | |
| 1602 | U06965 | Aphytis melinus mitochondrion 16S rRNA gene, partial | 0.005 | |
| 1603 | Z81014 | Human DNA sequence from cosmid U65A4, between | 0.005 | |
| | | markers DX5366 and DXS87 on chromosome X * | | |
| 1604 | X91923 | H. sapiens ECE-1 gene (exon 3) | 0.005 | |
| 1605 | Z29641 | Zea mays of USE gene encoding U3snRNA | 0.005 | |
| 1606 | L11670 | Human transmembrane glycoprotein (CD53) gene, exons 2 | 0.005 | |
| 1607 | U15605 | Nicotiana glutinosa virus resistance (N) gene, complete cds. | 0.005 | |
| 1608 | X57698 | A. thaliana DNA for acyl carrier protein (ACP) gene A1 | 0.005 | |
| 1609 | L81391 | Homo sapiens (subclone 2_a6 from P1 H39) DNA sequence | 0.005 | |
| 1610 | X81789 | H. sapiens mRNA for splicing factor SF3a60 | 0.005 | |
| 1611 | X82818 | H. sapiens PTP1C/HCP gene | 0.005 | |
| 1612 | U33465 | Drosophila melanogaster S element and flanking sequence, | 0.005 | |
| 1613 | Z75042 | S. cerevisiae chromosome XV reading frame ORF YOR134w | 0.005 | |
| 1614 | M98574 | Mouse T cell receptor rearranged alpha--chain variable | 0.005 | |
| | | region, N_region, joining region, and constant region | | |
| 1615 | X51907 | Human vimentin gene 5′ regulatoiy region | 0.005 | |
| 1616 | U50383 | Human retinoic acid-responsive protein (NN8-4AG) mRNA, | 0.005 | |
| | | complete cds | | |
| 1617 | M25140 | Human cardiac alpha-myosin heavy chain (MYH6) gene, | 0.005 | |
| | | exons 2, 3 and 4. | | |
| 1618 | X66062 | G. max tufA gene for chloroplast translation elongation | 0.005 | |
| 1619 | U16345 | Gallus gallus protein kinase gene (cPITSLRE) gene, exons 4 | 0.005 | |
| 1620 | AF012899 | Sambucus nigra ribosome inactivating protein precursor | 0.005 | |
| | | mRNA, complete cds | | |
| 1621 | U43834 | Saccharomyces cerevisiae chromosome IV lambda 3073 and | 0.005 | |
| | | flanking region extending into right telomere | | |
| 1622 | X13602 | Caldocellum saccharolyticum celB gene for | 0.005 | |
| | | cellobiohydrolase/endocellulase | | |
| 1623 | J04809 | Human cytosolic adenylate kinase (AK1) gene, complete | 0.005 | |
| 1624 | L49144 | Homo sapiens neuroendocrine-specific protein (NSP) gene, | 0.005 | |
| | | exons 1B and 3 | | |
| 1625 | L34028 | Plasmodium falciparum (clone HB3) heat shock protein 86 | 0.005 | |
| | | gene, complete cds. | | |
| 1626 | U50383 | Human retinoic acid-responsive protein (NN8-4AG) mRNA, | 0.005 | |
| | | complete cds | | |
| 1627 | U23829 | Lucilia cuprina peritrophin-95 precursor gene, partial cds. | 0.005 | |
| 1628 | AE000629.1 | Helicobacter pylori 26695 section 107 of 134 of the | 0.005 | |
| 1629 | M16446 | Human adenine phosphonibosyltransferase (APRT) gene, | 0.005 | |
| | | complete cds. | | |
| 1630 | U34605 | Human retinoic acid- and interferon-inducible 58 K protein | 0.005 | |
| | | RI58 mRNA, complete cds. | | |
| 1631 | M25140 | Human cardiac alpha-myosin heavy chain (MYH6) gene, | 0.005 | |
| | | exons 2, 3 and 4. | | |
| 1632 | X16426 | Mouse MHC (Qa) Q10-k gene for class I antigen | 0.005 | |
| 1633 | M83985 | Mouse phosphoprotein (F1-20) mRNA, complete cds. | 0.005 | |
| 1634 | U31850 | Human dystonin isoform 1 mRNA, partial cds | 0.005 | |
| 1635 | U31850 | Human dystonin isoform 1 mRNA, partial cds | 0.005 | |
| 1636 | X64615 | O. berteriana chloroplast ORF80 (exon 1 and 2) and | 0.005 | |
| | | ORF2280 sequences | | |
| 1637 | X52647 | E. coli dbpA gene for DEAD box protein A | 0.005 | |
| 1638 | D90773 | E. coli genomic DNA, Kohara clone #262(30.3-30.5 min.) | 0.005 | |
| 1639 | M62946 | S. glaucescens novel deletion/rearrangement sequence, | 0.005 | |
| | | partial sequence. | | |
| 1640 | M88597 | Saccharomyces cerevisiae STP1 gene, complete cds. | 0.005 | |
| 1641 | L31521 | Homo sapiens (clone HG52) Z-crystallin/quinone reductase | 0.005 | |
| | | (CRYZ) gene sequence. | | |
| 1642 | D79986 | Human mRNA for KIAA0164 gene, complete cds | 0.004 | |
| 1643 | AC002183 | Homo sapiens (subclone 2_h8 from BAC H111) DNA | 0.004 | |
| 1644 | Z29641 | Zea mays of USE gene encoding U3snRNA | 0.004 | |
| 1645 | L22415 | Homo sapiens DNA sequence, repeat region. | 0.004 | |
| 1646 | U17357 | Chlamydomonas reinhardtii chloroplast 30S ribosomal | 0.004 | |
| | | protein S4 (rps4) gene, complete cds. | | |
| 1647 | M67465 | Rat 3-beta-hydroxysteroid dehydrogenase/delta-5-delta-4- | 0.004 | |
| | | ene-isomerase mRNA, complete cds. | | |
| 1648 | U08421 | Murine rotavirus EC outer capsid protein VP4 gene, | 0.004 | |
| 1649 | Z17089 | H. sapiens (D3S1309) DNA segment containing | 0.002 | |
| 1650 | L35531 | Human Alu repeat region. | 0.002 | |
| 1651 | M55120 | Human cystic fibrosis transmembrane conductance regulator | 0.002 | |
| | | (CFTR) gene, exon 14b | | |
| 1652 | J00922 | Gallus gallus ovalbumin (Y) gene, complete cds. | 0.002 | |
| 1653 | U69695 | Mus musculus hyaluronan synthase homolog mRNA, | 0.002 | |
| 1654 | L15248 | Human (clone: pHyTM1/60(R)) DNA sequence. | 0.002 | |
| 1655 | L35676 | Homo sapiens (subclone H8 2_e7 from P1 35 H5 C8) DNA | 0.002 | |
| 1656 | L48612 | Theileria parva (clone pTprUgB) ORF genes, partial cds. | 0.002 | |
| 1657 | Y08925 | P. falciparum aarp3 gene, exon | 0.002 | |
| 1658 | M60441 | Sheep lambda immunoglobulin V gene. | 0.002 | |
| 1659 | X91857 | Piromyces sp. mRNA for mannanase A | 0.002 | |
| 1660 | Z35948 | S. cerevisiae chromosome II reading frame ORF YBR079c | 0.002 | |
| 1661 | X16277 | Human gene for ornithine decarboxylase ODC (EC 4.1.1.17) | 0.002 | |
| 1662 | X78608 | G. gallus genomic DNA repeat region, clone 9C2 | 0.002 | |
| 1663 | U48449 | Human skeletal muscle ryanodine receptor gene | 0.002 | |
| 1664 | X51875 | Human breakpoint in translocation V-kappa gene region | 0.002 | |
| | | (WB) (partial) (537 bp) | | |
| 1665 | Z24205 | H. sapiens (D12S348) DNA segment containing | 0.002 | |
| 1666 | U85612 | Mus musculus cartilage-derived retinoic acid-sensitive | 0.002 | |
| | | protein/melanoma inhibitory activity protein gene, complete | | |
| 1667 | M61829 | Human alpha-1,3-mannosyl-glycoprotein beta-1, 2-N- | 0.002 | |
| | | acetylglucosaminyltransferase (MGAT) gene, complete cds. | | |
| 1668 | M61829 | Human alpha-1,3-mannosyl-glycoprotein beta-1, 2-N- | 0.002 | |
| | | acetylglucosaminyltransferase (MGAT) gene, complete cds. | | |
| 1669 | V00571 | Human gene encoding prepro form of corticotropin releasing | 0.002 | |
| 1670 | D29760 | Candida maltosa gene for chitin synthase 1, partial sequence | 0.002 | |
| 1671 | AJ001817 | Dama sp. mRNA for bone morphogenetic protein 2 | 0.002 | |
| 1672 | Z23575 | H. sapiens (D17S926) DNA segment containing | 0.002 | |
| 1673 | X59359 | T. marmorata mRNA for acetylcholinesterase | 0.002 | |
| 1674 | D12519 | Rat SAP gene for synaptotagmin associated 35kDa protein | 0.002 | |
| 1675 | U88534 | Mus musculus glucose-6-phosphate dehydrogenase protein, | 0.002 | |
| | | exons 10, 11 and partial cds | | |
| 1676 | Z24391 | H. sapiens (D11S1350) DNA segment containing | 0.002 | |
| 1677 | M31773 | Murine B cell 1 (mb-1) gene, complete cds. | 0.002 | |
| 1678 | U28014 | Human cysteine protease (ICErel-II) mRNA, complete cds. | 0.002 | j |
| 1679 | M33518 | Human HLA-B-associated franscript 2 (BAT2) gene, 3′ end. | 0.002 | |
| 1680 | U70983 | Pseudomonas aeruginosa heptosyl transferase II | 0.002 | |
| | | Pseudomonas aeruginosa heptosyl transferase II (rfaF) gene, | | |
| 1681 | Z74854 | S. cerevisiae chromosome XV reading frame ORF YOL112w | 0.002 | |
| 1682 | M13498 | Mouse renal kallikrein gene mGK-6, exon 1 | 0.002 | |
| 1683 | U17382 | Streptococcus pyogenes putative multiple membrane domain | 0.002 | |
| | | protein gene, complete cds. | | |
| 1684 | U14189 | Plasmodium falciparum MCP1 mRNA, complete cds | 0.001 | |
| 1685 | M27314 | Saccharomyces cerevisiae mitochondrial petite mutant BB5 | 0.001 | |
| | | origin of replication DNA. | | |
| 1686 | M64089 | Diclyostelium discoideum calmodulin (calA) gene, complete | 0.001 | |
| 1687 | J05138 | Rabbit calcium binding protein (calreticulin) mRNA, | 0.0009 | |
| 1688 | U92017 | Human clone 199288 defective mariner transposon Hsmar2 | 0.0008 | |
| | | mRNA sequence | | |
| 1689 | AJ011915 | Homo sapiens mRNA for synaptosome associated protein of | 0.0008 | |
| | | 23 kilodaltons, isoform A | | |
| 1690 | AJ223734 | Sus scrofa SCAMP1 gene, exon 1 and joined CDS | 0.0008 | |
| 1691 | Y08924 | P. falciparum mRNA for AARP2 protein | 0.0006 | |
| 1692 | X58139 | Human coxVIb gene, last exon and flanking sequence | 0.0006 | |
| 1693 | U47853 | Araneus diadematus fibroin-1 (ADF-1) mRNA, partial cds | 0.0006 | |
| 1694 | L34027 | Plasmodium falciparum (clone Dd2) heat shock protein 86 | 0.0006 | |
| | | gene, complete cds. | | |
| 1695 | D88271 | Human (lambda) DNA for immunogloblin light chain | 0.0006 | |
| 1696 | AC001546 | Homo sapiens (subclone 2_b1 from P1 H69) DNA sequence | 0.0006 | |
| 1697 | Z96325 | H. sapiens telomeric DNA sequence, clone 16QTEL024, | 0.0006 | |
| | | read 16QTELOO024.seq | | |
| 1698 | U14974 | Saccharomyces cerevisiae Nmd2p (NMD2) gene, complete | 0.0006 | |
| | | cds. > :: gb\|I70400\|I70400 Sequence 1 from patent US | | |
| 1699 | AB002331 | Human mRNA for KIAA0333 gene, partial cds | 0.0006 | |
| 1700 | AE000464 | Escherichla coli K-12 MG1655 section 354 of 400 of the | 0.0006 | |
| | | complete genome | | |
| 1701 | Z79060 | H. sapiens flow-sorted chromosome 6 HindIII fragment, | 0.0005 | |
| 1702 | Z60233 | H. sapiens CpG island DNA genomic Mse1 fragment, clone | 0.0005 | |
| | | 197c9, reverse read cpg197c9.rt1a | | |
| 1703 | U15018 | Dugbe virus L protein gene, complete cds | 0.0005 | |
| 1704 | X77607 | H. sapiens genomic DNA (leukocyte), corresponding to the | 0.0005 | |
| | | integration site of HPV 6a DNA in a tonsillar carcinoma | | |
| 1705 | M59428 | T. thermophila ribosomal protein L37 (L37) mRNA, | 0.0005 | |
| 1706 | M59428 | T. thermophila ribosomal protein L37 (L37) mRNA, | 0.0005 | |
| 1707 | AC002219 | Homo sapiens (subclone 2_d11 from P1 H43) DNA | 0.0005 | |
| 1708 | X95276 | P. falciparum complete gene map of plastid-like DNA (IR-B) | 0.0005 | |
| 1709 | L18972 | Homo sapiens anonymous gene, complete cds | 0.005 | |
| 1710 | U96974 | Homo sapiens MET proto-oncogene, intron 5, 3′ end | 0.0005 | |
| 1711 | Z60916 | H. sapiens CpG island DNA genoniic Mse1 fragment, clone | 0.0005 | |
| | | 39a5, forward read cpg39a5.ft1c | | |
| 1712 | X99587 | A. brasilense ipdC, gltX & cysS genes | 0.0005 | |
| 1713 | J03998 | P. falciparum glutamic acid-rich protein gnen, complete cds. | 0.0005 | |
| 1714 | AE000464 | Escherichla coli K-12 MG1655 section 354 of 400 of the | 0.0005 | |
| | | complete genome | | |
| 1715 | U21730 | Human 5′-nucleotidase (CD73) gene, partial cds. | 0.0004 | |
| 1716 | M22970 | Human pancreatic phospholipase A-2 (PLA-2) gene, exons 1 | 0.0003 | |
| 1717 | U37022 | Human cyclin-dependent kinase 4 (CDK4) gene, complete | 0.0002 | |
| 1718 | AC001517 | Homo sapiens (subclone 1_g5 from P1 H49) DNA sequence | 0.0002 | |
| 1719 | X90383 | A. thaliana DNA for Y13 gene | 0.0002 | |
| 1720 | NM_003407.1 | Homo sapiens zinc finger protein homologous to Zfp-36 in | 0.0002 | |
| | | mouse (ZFP36) mRNA > :: gb\|M92843\|HUMG0S24A | | |
| | | H. sapiens zinc finger transcriptional regulator mRNA, | | |
| 1721 | M86528 | Human neurotrophin-4 (NT-4) gene, complete cds. | 0.0002 | |
| 1722 | M86528 | Human neurotrophin-4 (NT-4) gene, complete cds. | 0.0002 | |
| 1723 | U19241 | Homo sapiens interferon-gamma receptor alpha chain gene | 0.0002 | |
| 1724 | M15205 | Human thymidine kinase gene, complete cds, with clustered | 0.0002 | |
| | | Alu repeats in the introns. | | |
| 1725 | M87510 | Mouse Y specific region of AC11 DNA sequence, LINE | 0.0002 | |
| | | repeat and Bkm satellite. | | |
| 1726 | Z84723 | Human DNA sequence from phage LAW2 from a contig | 0.0002 | |
| | | from the tip of the short arm of chromosome 16, spanning | | |
| | | 2Mb of 16p13.3 Contains Interleukin 9 receptor pseudogene | | |
| 1727 | X01392 | Human apolipoprotein CIII gene and apo AI-apo CIII | 0.0002 | |
| 1728 | Z92910 | H. sapiens HFE (HLA-H) gene | 0.0002 | |
| 1729 | D87001 | Human (lambda) DNA for immunoglobulin light chain | 0.0002 | |
| 1730 | M35612 | Yeast (S. cerevisiae) mitochondrial autonomously replicating | 0.0002 | |
| | | sequence DNA | | |
| 1731 | Z16956 | H. sapiens (D2S154) DNA segment containing | 0.0002 | |
| 1732 | L42456 | Mus musculus TGF-t gene, promoter region and exon I. | 0.0002 | |
| 1733 | X90383 | A. thaliana DNA for Y13 gene | 0.0002 | |
| 1734 | L35657 | Homo sapiens (subclone H8 5_a10 from P1 35 H5 C8) DNA | 0.0002 | |
| 1735 | M57902 | Mouse transforming growth factor-beta-1 | 0.0002 | |
| 1736 | U67167 | Homo sapiens intestinal mucin (MUC2) gene, promoter | 0.0002 | |
| | | region and partial cds | | |
| 1737 | NM_002824.1 | Homo sapiens parathymosin (PTMS) mRNA > :: | 0.0002 | |
| | | gb\|M24398\|HUMTHYP Human parathymosin MRNA, | | |
| 1738 | J05138 | Rabbit calcium binding protein (calreticulin) mRNA, | 0.0002 | |
| 1739 | U96566 | HIV-1 patient JO population variant JOS17 from USA, | 0.0002 | |
| | | envelope glycoprotem, C2-V5 region (env) gene, partial cds | | |
| 1740 | AF043461 | Homo sapiens I-REL gene, exon 9 | 9e-005 | |
| 1741 | M96943 | Human profilaggrin gene exons 1-3, 5′ end. | 8e-005 | |
| 1742 | S45332 | erythropoietin receptor [human, placental, Genomic, 8647 | 7e-005 | |
| 1743 | S45332 | erythropoietin receptor [human, placental, Genomic, 8647 | 7e-005 | |
| 1744 | L28125 | Podospora anserina beta transducin-like protein | 7e-005 | |
| 1745 | U53017 | Human microsatellite marker sJCW13 | 6e-005 | |
| 1746 | M86528 | Human neurotrophin-4 (NT-4) gene, complete cds. | 6e-005 | |
| 1747 | D17554 | Human mRNA for DNA-binding protein, TAXREB107, | 6e-005 | |
| 1748 | U26556 | Human ferritin H (FTHL13) pseudogene. | 6e-005 | |
| 1749 | Z79060 | H. sapiens flow-sorted chromosone 6 HindIII fragment, | 6e-005. | |
| 1750 | AC001460 | Homo sapiens (subclone 2_f4 from BAC H107) DNA | 6e-005 | |
| 1751 | AC001033 | Homo sapiens (subclone 2_c2 from P1 H48) DNA sequence | 6e-005 | |
| 1752 | AF007883 | Homo sapiens MHC class II HLA-DRB1 (HLA-DRB1*10) | 6e-005 | |
| | | intron 1 sequence | | |
| 1753 | U12971 | Tetrahymena thermophila CU428.1VII micronuclear M | 6e-005 | |
| 1754 | X83872 | H. vulgaris mRNA for cAMP response element binding | 6e-005 | |
| 1755 | Z73360 | Human DNA sequence from cosmid 92M18, BRCA2 gene | 5e-005 | |
| | | region chromosome 13q12-13 | | |
| 1756 | X85116 | H. sapiens epb72 gene exon 1 | 5e-005 | |
| 1757 | L81639 | Homo sapiens (subclone 2_b2 from P1 H39) DNA sequence | 3e-005 | |
| 1758 | D16184 | Chicken mRNA for nuclear b-Zip protein MafF, complete | 3e-005 | |
| 1759 | AF100694 | Mus musculus Pontin52 mRNA, complete cds | 3e-005 | |
| 1760 | Z30978 | G. gorilla DNA for Mhc Alu elements | 3e-005 | |
| 1761 | L23429 | Canis beta-galactosides-binding lectin (LGALS3) mRNA, | 2e-005 | |
| 1762 | X06559 | Human interferon-inducible gene IFI-56K 5′ region (56 kDa | 2e-005 | |
| | | coding capacity of unknown function) | | |
| 1763 | AC001546 | Homo sapiens (subclone 2_b1 from P1 H69) DNA sequence | 2e-005 | |
| 1764 | L35658 | Homo sapiens (subclone H8 9_d12 from P1 35 H5 C8) | 2e-005 | |
| 1765 | D16472 | Human mRNA, Xq terminal portion | 2e-005 | |
| 1766 | M83665 | Human high mobility group 2 protein (HMG-2) gene | 2e-005 | |
| 1767 | U07977 | Human T cell receptor beta (TCRBV9S1, TCRBV7S1) | 2e-005 | |
| | | genes, TCRBV inserted and TCRBV deleted haplotype, | | |
| 1768 | D83227 | Populus nigra gene for extensin like protein, complete cds | 2e-005 | |
| 1769 | U42053 | Mustela vison microsatellite repeat (Mvi 24). | 2e-005 | |
| 1770 | L29339 | Homo sapiens Na+/glucose cotransporter (SGLT1) gene, | 2e-005 | |
| | | exon 15 and complete cds. | | |
| 1771 | Z33620 | M. musculus (Balb/c) GATA-3 gene (partial) | 2e-005 | |
| 1772 | D88271 | Human (lambda) DNA for immunogloblin light chain | 2e-005 | |
| 1773 | AD000833 | Homo sapiens DNA from chromosome 19-cosmid f19399 | 2e-005 | |
| | | (˜17 kb EcoRI restriction fragment) | | |
| 1774 | U36755 | Human thrombin receptor (F2R) gene, 5′ region and partial | 2e-005 | |
| 1775 | AC002252 | Homo sapiens (subclone 1_g7 from BAC H76) DNA | 2e-005 | |
| 1776 | AE000464 | Escherichia coli K-12 MG1655 section 354 of 400 of the | 2e-005 | |
| | | complete genome | | |
| 1777 | X94768 | H. sapiens RP3 gene (XLRP gene 3) | 1e-005 | |
| 1778 | U57058 | Human WD protein IR10 pre-mRNA, partial cds | 9e-006 | |
| 1779 | AC001603 | Homo sapiens (subclone 2_a9 from PAC H92) DNA | 8e-006 | |
| 1780 | Z47046 | Human cosmid QLL2C9 from Xq28 | 7e-006 | |
| 1781 | U93275 | Mus musculus glucokinase gene, 5′ flanking region | 7e-006 | |
| 1782 | X60653 | human Histone H3.3 pseudogene (CIR-456) | 7e-006 | |
| 1783 | L81583 | Homo sapiens (subclone 3_g2 from P1 H11) DNA sequence | 6e-006 | |
| 1784 | L13381 | Plasmodium falciparum HB3\W2 transport protein | 6e-006 | |
| 1785 | U97576 | Homo sapiens TRE17 oncogene-associated GOS19- | 6e-006 | |
| | | 2/MIP1alpha gene, downstream sequence | | |
| 1786 | Y11204 | V. carteri gene encoding volvoxopsin | 6e-006 | |
| 1787 | Z16794 | H. sapiens (D4S409) DNA segment containing (CA) repeat; | 6e-006 | |
| | | clone AFM183xd6; single read | | |
| 1788 | D83737 | Human coagulation factor XII gene, intron 2 | 6e-006 | |
| 1789 | X04871 | Paramecium primaurelia macronuc1ear DNA telomeric | 6e-006 | |
| 1790 | M14292 | Human L1Heg repetitive element from the intergenic region | 6e-006 | |
| | | of the epsilon and G-gamnma globin genes. | | |
| 1791 | NM_003734.1 | Homo sapiens amine oxidase, copper containing 3 | 6e-006 | |
| | | gb\|U39447\|HSU39447 Human placenta copper monamine | | |
| | | oxidase mRNA, complete cds | | |
| 1792 | M27147 | Human alpha-2-plasmin inhibitor, allele A, 5′ end. | 6e-006 | |
| 1793 | X86012 | Human DNA sequence from intron 22 of the factor VIII | 6e-006 | |
| | | gene, Xq28. Contains the end of a 9.5 kb repeated region, | | |
| | | int22h-1, involed in many cases of haemophilia | | |
| 1794 | M33216 | Human aortic-type smooth muscle alpha-actin | 3e-006 | |
| 1795 | Z83334 | H. sapiens RPS3a gene | 2e-006 | |
| 1796 | M57682 | Rat brain calcium channel alpha-1 subunit mRNA, complete | 2e-006 | |
| 1797 | M19817 | Human apolipoprote1n B-100 (apoB) gene, intron J. | 2e-006 | |
| 1798 | Z24068 | H. sapiens (D22S427) DNA segment containing | 2e-006 | |
| 1799 | Z50155 | X. laevis mRNA for insulin-like growth factor I receptor | 2e-006 | |
| 1800 | Y12839 | H. sapiens BH30 mRNA | 2e-006 | |
| 1801 | Z70041 | Human DNA sequence from cosmid U39H5, between | 2e-006 | |
| | | markers DXS6791 and DXS8038 on chromosome X | | |
| 1802 | Z80128 | H. sapiens CACNL1A4 gene, exons 16 and 17 | 2e-006 | |
| 1803 | U80893 | Mus musculus CAG trinucleotide repeat mRNA, partial | 2e-006 | |
| 1804 | Z63192 | H. sapeins CpG island DNA genomic Mse1 fragment, clone | 2e-006 | |
| | | 7a7, forward read cpg7a7.ft1d | | |
| 1805 | U72964 | Human hepatocyte nuclear factor 4-alpha gene, exon 5 | 2e-006 | |
| 1806 | AC002183 | Homo sapiens (subclone 2_h8 from BAC H111) DNA | 2e-006 | |
| 1807 | S73557 | annexin II = 36 kDa calcium-dependent phospholipid-binding | 2e-006 | |
| | | protein [rats, RBL-2H3 basophilic leukemia cells, mRNA, | | |
| 1808 | U79258 | Human clone 23732 mRNA, partial cds | 8e-007 | |
| 1809 | Z62146 | H. sapiens CpG island DNA genomic Mse1 fragment, clone | 8e-007 | |
| | | 64b2, forward read cpg64b2.ft1a | | |
| 1810 | U44381 | Human tissue inhibitor of metalloprotemases-2 | 8e-007 | |
| 1811 | Z65575 | H. sapiens CpG island DNA genomic Mse1 fragment; clone | 7e-007 | |
| | | 47c5, reverse read cpg47c5.rt1a | | |
| 1812 | J03764 | Human, plasminogen activator inhibitor-1 gene, exons 2 to | 7e-007 | |
| 1813 | X75349 | H. sapiens 5′flanking DNA for clotting factor IX | 7e-007 | |
| 1814 | Z73360 | Human DNA sequence from cosmid 92M18, BRCA2 gene | 7e-007 | |
| | | region chromosome 13q12-13 | | |
| 1815 | Z73360 | Human DNA sequence from cosmid 92M18, BRCA2 gene | 7e-007 | |
| | | region chromosome 13q12-13 | | |
| 1816 | X77624 | H. sapiens simple sequence clone pg2m3, 5′ flank and repeats | 7e-007 | |
| 1817 | S46857 | SCL/TCL5/tal-1 = stem-cell leukemia {germline chromosome | | |
| | | 3 translocation/deletion breakpoint} [human, bone marrow | | |
| | | cells, Genomic Mutant, 239 nt] | | |
| 1818 | J03998 | P. falciparum glutamic acid-rich protein gnen, complete cds. | 7e-007 | |
| 1819 | AF012899 | Sambucus nigra ribosome inactivating protein precursor | 3e-007 | |
| | | mRNA, complete cds | | |
| 1820 | U47654 | Human pyruvate kinase PK-R gene, partial cds, and pyruvate | 3e-007 | |
| | | kinase PK-L gene, complete cds. | | |
| 1821 | U78096 | Human macrophage colony stimulating factor receptor (c- | 3e-007 | |
| | | fms) gene, exon 1A, 2 and partial cds | | |
| 1822 | L76927 | Human galactokinase (GALK1) gene, complete cds | 3e-007 | |
| 1823 | U22086 | Ursus americanus clone G10H GT and ATTT microsatellite | 3e-007 | |
| 1824 | J03069 | Human MYCL2 gene, complete cds. | 3e-007 | |
| 1825 | X82640 | D. melanogaster mRNA for alpha 1,2 mannosidase | 3e-007 | |
| 1826 | U18671 | Human Stat2 gene, complete cds. | 2e-007 | |
| 1827 | L02935 | Human major breakpoint cluster region (BCR) gene, exons 1- | 2e-007 | |
| | | 3 and repeat regions. | | |
| 1828 | L04193 | Human lens membrane protein (mp19) gene, exon 11. | 2e-007 | |
| 1829 | AC001050 | Homo sapiens (subclone 3_e9 from P1 H55) DNA sequence | 2e-007 | |
| 1830 | AF012899 | Sambucus nigra ribosome inactivating protein precursor | 2e-007 | |
| | | mRNA, complete cds | | |
| 1831 | AF012899 | Sambucus nigra ribosome inactivating protein precursor | 9e-008 | |
| | | mRNA, complete cds | | |
| 1832 | L78776 | Homo sapiens (subclone 2_a7 from P1 H49) DNA sequence | 9e-008 | |
| 1833 | U41315 | Human ring zinc-finger protein (ZNF127-Xp) gene and 5′ | 9e-008 | |
| | | flanking sequence. | | |
| 1834 | X95586 | H. sapiens MB1 gene | 9e-008 | |
| 1835 | M33387 | Human debrisoquine 4-hydroxylase (CYP2D8P) and | 9e-008 | |
| 1836 | U09954 | Human ribosomal protein L9 gene, 5′ region and complete | 8e-008 | |
| 1837 | Z15030 | H. sapiens gene for ventricular myosin light chain 2 > :: | 8e-008 | |
| | | gb\|L01652\|HUMVMLC Human ventricular myosin light | | |
| | | chain 2 gene, seven exons. | | |
| 1838 | AF012899 | Sambucus nigra ribosome inactivating protein precursor | 8e-008 | |
| | | mRNA, complete cds | | |
| 1839 | AF012899 | Sambucus nigra ribosome inactivating protein precursor | 8e-008 | |
| | | mRNA, complete cds | | |
| 1840 | Z77974 | H. sapiens flow-sorted chromosome 6 HindIII fragment, | 8e-008 | |
| 1841 | AF012899 | Sambucus nigra ribosome inactivating protein precursor | 8e-008 | |
| | | mRNA, complete cds | | |
| 1842 | L81802 | Homo sapiens (subclone 1_c12 from P1 H31) DNA | 8e-008 | |
| 1843 | D87001 | Human (lambda) DNA for immunoglobulin light chain | 8e-008 | |
| 1844 | Z23971 | H. sapiens (D2S338) DNA segment containing (CA) repeat; | 8e-008 | |
| | | clone AFM276zf5; single read | | |
| 1845 | X89398 | H. sapeins ung gene for uracil DNA-glycosylase | 3e-008 | |
| 1846 | Z68212 | Phocine Herpesvirus 1 DNA (clone 4; 280 bp) | 3e-008 | |
| 1847 | M85145 | Human tumor necrosis factor receptor, 3′ flank. | 3e-008 | |
| 1848 | M17919 | Human DNA with homology to EBV IR3 repeat, clone Hu3. | 3e-008 | |
| 1849 | M21339 | Human non-histone chromosomal protein HMG-14 gene, | 3e-008 | |
| | | complete cds. | | |
| 1850 | Z69655 | Human DNA sequence from cosmid L98A6, Huntington's | 3e-008 | |
| | | Disease Region, chromosome 4p16.3 | | |
| 1851 | S83526 | red photopigment gene {Alu repeat region, long intron 1} | | |
| | | [human, peripheral blood leucocytes, Genomic, 1987 net] | | |
| 1852 | M11809 | Human (2′-5′) oligo A synthetase E gene, exon 7 and flanks. | 3e-008 | |
| 1853 | X94768 | H. sapiens RP3 gene (XLRP gene 3) | 3e-008 | |
| 1854 | X62025 | H. sapiens rod cG-PDE G gene for 3′,5′-cyclic nucleotide | 3e-008 | |
| | | phosphodiesterase | | |
| 1855 | NM_000694.1 | Homo sapiens aldehyde dehydrogenase 7 (ALDH7) mRNA | 3e-008 | |
| | | > :: gb\|U10868\|HSU10868 Human aldehyde dehydrogenase | | |
| | | ALDH7 mRNA, complete cds. | | |
| 1856 | U22086 | Ursus americanus clone G10H GT and ATTT microsatellite | 3e-008 | |
| 1857 | AC001174 | Homo sapiens (subclone 1_e2 from BAC H94) DNA | 3e-008 | |
| 1858 | X69908 | H. sapiens gene for mitochondrial ATP synthase c subunit | 2e-008 | |
| 1859 | X91233 | H. sapiens IL15 gene | 2e-008 | |
| 1860 | M61835 | Human lactase phlorizin hydrolase (LCT) gene, exon 2. | 1e-008 | |
| 1861 | M29324 | Mouse L1Md-A13 repetitive sequence. | 1e-008 | |
| 1862 | AF021806 | Rattus norvegicus connexin 40 (GJA5) mRNA, complete cds | 1e-008 | |
| 1863 | AB002584 | Rattus norvegicus mRNA for beta-alanine-pyruvate | 1e-008 | |
| | | aminotransferase, complete cds | | |
| 1864 | Z54147 | Human DNA sequence from cosmid L129H7, Huntington's | 9e-009 | |
| | | Disease Region, chromosome 4p16.3 contains CpG island | | |
| 1865 | AF012899 | Sambucus nigra ribosome inactivating protein precursor | 9e-009 | |
| | | mRNA, complete cds | | |
| 1866 | Z54349 | H. sapiens MN/CA9 GENE | 8e-009 | |
| 1867 | M21487 | Human muscle creatine kinase gene (CKMM), 5′ flank. | 3e-009 | |
| 1868 | U02993 | Human cytochrome P450 (Cyp1A2) gene, 5′ region. | 3e-009 | |
| 1869 | Z78893 | H. sapiens flow-sorted chromosome 6 HindIII fragment, | 3e-009 | |
| 1870 | U89387 | Human RNA polymerase II subunit hsRPB4 gene, complete | 3e-009 | |
| 1871 | X57413 | Mouse mRNA for transforming growth factor-beta2 | 3e-009 | |
| 1872 | Z94828 | G. gallus microsatellite DNA (LEI0260 | 3e-009 | |
| 1873 | D26067 | Human mRNA for KIAA0033 gene, partial cds | 3e-009 | |
| 1874 | AB001914 | Homo sapiens PACE4 gene, exon 23-25, complete cds | 3e-009 | |
| 1875 | Z75894 | Human DNA sequence from cosmid U61F10, between | 3e-009 | |
| | | markers DXS366 and DXS87 on chromosome X contains | | |
| 1876 | AC001443 | Homo sapiens (subclone 2_f10 from BAC 2913 | 3e-009 | |
| 1877 | M96851 | Human CpG island containing upstream sequence | 3e-009 | |
| 1878 | D64108 | Human mRNA for DMC1 homologue, complete cds | 3e-009 | |
| 1879 | S80861 | {junction region} [human, KOPT-K1 cells, T-cell acute | 3e-009 | |
| | | lymphoblastic leukemia patient, Genomic, 895 nt] | | |
| 1880 | U79776 | Mus musculus ajuba (Ajuba) mRNA, complete cds | 2e-009 | |
| 1881 | S75283 | thyrotropin-releasing hormone receptor [human, Genomic, | 1e-009 | |
| | | 2312 nt, segment 2 of 2] | | |
| 1882 | X14445 | Human int-2 proto-oncogene | 1e-009 | |
| 1883 | J03764 | Human, plasminogen activator inhibitor-1 gene, exons 2 to | 1e-009 | |
| 1884 | L36911 | Pig microsatellite DNA (CA repeat) | 1e-009 | |
| 1885 | U79746 | Homo sapiens serotonin transporter (hSERT) gene, promoter | 9e-010 | |
| | | region, exons 1B and 2, and partial cds | | |
| 1886 | X56668 | Human DNA for calretinin exon 1 | 9e-010 | |
| 1887 | AF012899 | Sambucus nigra ribosome inactivating protein precursor | 9e-010 | |
| | | mRNA, complete cds | | |
| 1888 | X77738 | H. sapiens red cell anion exchanger (EPB3, AE1, Band 3) | 4e-010 | |
| | | gene, 3′ region | | |
| 1889 | L02897 | Dog nonerythroid beta-spectrin mRNA, 3′ end. | 3e-010 | |
| 1890 | D45198 | Human mRNA for template acyivating factor-I alpha, | 3e-010 | |
| 1891 | X04981 | H. sapiens gene for lecithin-cholesterol acyltransferase | 3e-010 | |
| 1892 | M14292 | Human L1Heg repetitive element from the intergenic region | 3e-010 | |
| | | of the epsilon and G-gamma globin genes. | | |
| 1893 | X14448 | Human GLA gene for alpha-D-galactosidase A (EC | 3e-010 | |
| 1894 | Z96616 | H. sapiens telomeric DNA sequence, clone 4QTEL025, read | 3e-010 | |
| | | 4QTELOO025.seq | | |
| 1895 | M12901 | Human c-mos pseudogene with Alu repeat insertions, partial | 2e-010 | |
| 1896 | Z68885 | Human DNA sequence from cosmid L21F12B, Huntington's | 1e-010 | |
| | | Disease Region, chromosome 4p16.3, contains EST | | |
| 1897 | L77036 | Homo sapiens (subclone 5_d9 from P1 H19) DNA | 1e-010 | |
| 1898 | Z58927 | H. sapiens CpG island DNA genomic Mse1 fragment, clone | 1e-010 | |
| | | 116g2, reverse read cpg116g2.rt1a | | |
| 1899 | L77036 | Homo sapiens (subclone 5_d9 from P1 H19) DNA | 1e-010 | |
| 1900 | Z79007 | H. sapiens flow-sorted chromosome 6 HindIII fragment, | 1e-010 | |
| 1901 | Z75891 | Human DNA sequence from cosmid F45C1 on chromosome | 1e-010 | |
| 1902 | Z72930 | S. cerevisiae chromosome VII reading frame ORF YGR145w | 1e-010 | |
| 1903 | Z36111 | S. cerevisiae chromosome II reading frame ORF YBR242w | 5e-011 | |
| 1904 | Z54147 | Human DNA sequence from cosmid L129H7, Huntington's | 4e-011 | |
| | | Disease Region, chromosome 4p16.3 contains CpG island | | |
| 1905 | X87579 | H. sapiens CD4 gene | 4e-011 | |
| 1906 | U43604 | Human unidentified mRNA, partial sequence. | 4e-011 | |
| 1907 | U08024 | Human clone A dehydroepiandrosterone sulfotransferase | 4e-011 | |
| | | (STD) mRNA, complete cds. | | |
| 1908 | M27825 | B. lactucae heat shock protein 70 (hsp70) gene, complete | 4e-011 | |
| 1909 | Z15026 | H. sapiens genes for tumor necrosis factor (Tnfa) and | 3e-011 | |
| | | lymphotoxine (Tnfb) | | |
| 1910 | Z67997 | Human DNA sequence from cosmid L206D7, Huntington's | 3e-011 | |
| | | Disease Region, chromosome 4p16.3 | | |
| 1911 | AC001046 | Homo sapiens (subclone 3_f2 from P1 H54) DNA sequence | 2e-011 | |
| 1912 | Z84518 | H. sapiens flow-sorted chromosome 6 HindIII fragment, | 1e-011 | |
| 1913 | M59709 | Human carcinoembryonic antigen (CEA) gene, exon 10. | 1e-011 | |
| 1914 | L35670 | Homo sapiens (subclone H8 10_g5 from P1 35 H5 C8) | 1e-011 | |
| 1915 | AF012899 | Sambucus nigra ribosome inactivating protein precursor | 1e-011 | |
| | | mRNA, complete cds | | |
| 1916 | Z96209 | H. sapiens telomeric DNA sequence, clone 12PTEL055, read | 1e-011 | |
| | | 12PTELOO055.seq | | |
| 1917 | U34052 | Human Down Syndrome region of chromosome 21, genomic | 1e-011 | |
| | | sequence, clone A35A7-1A2 | | |
| 1918 | Z96489 | H. sapiens telomeric DNA sequence, clone 21QTEL007, | 1e-011 | |
| | | read 21QTELOO007.seq | | |
| 1919 | AC001036 | Homo sapiens (subclone 2_f7 from P1 H48) DNA sequence | 1e-011 | |
| 1920 | L42098 | Homo sapiens (subclone 5_c7 from P1 H22) DNA sequence. | 6e-012 | |
| 1921 | X93341 | H. sapiens mitochondrial control region DNA | 5e-012 | |
| 1922 | D26141 | Human NF1 gene homologue | 4e-012 | |
| 1923 | U80228 | Human clotting factor VIII gene, intron 20 and exon 21, | 4e-012 | |
| | | partial sequence | | |
| 1924 | U16812 | Human Bak-2 gene, complete cds. | 4e-012 | |
| 1925 | Z68758 | Human DNA sequence from cosmid nN85E10 on | 4e-012 | |
| | | chromosome 22q11.2-qter | | |
| 1926 | AC001449 | Homo sapiens (subclone 2_f10 from P1 H103) DNA | 4e-012 | |
| 1927 | D50561 | Human DNA, replication enhancing element (REEl) | 4e-012 | |
| 1928 | Z96387 | H. sapiens telomeric DNA sequence, clone 18PTEL033, read | 1e-012 | |
| | | 18PTELOO033.seq | | |
| 1929 | AF004338 | Homo sapiens 16S ribosomal RNA, mitochondrial gene, | 1e-012 | |
| | | partial sequence | | |
| 1930 | M15360 | Human transposon-like element (THE) p2 solo LTR with | 1e-012 | |
| | | inserted Alu element. | | |
| 1931 | L81577 | Homo sapiens (subclone 3_h8 from P1 H11) DNA sequence | 1e-012 | |
| 1932 | U56979 | Human complement factor H precursor (Cfh) gene, partial | 1e-012 | |
| 1933 | U14550 | Human sialyltransferase SThM (sthm) mRNA, complete cds. | 1e-012 | |
| 1934 | M31061 | Human ornithine decarboxylase gene, complete cds. | 1e-012 | |
| 1935 | U06752 | Rattus norvegicus Fisher 344 pre-sialomucin complex | 1e-012 | |
| | | (pSMC) mRNA, repeat sequences 10-14, partial cds. | | |
| 1936 | U06752 | Rattus norvegicus Fisher 344 pre-sialomucin complex | 1e-012 | |
| | | (pSMC) mRNA, repeat sequences 10-14, partial cds. | | |
| 1937 | L29096 | Homo sapiens oriP binding protein (OBP-2) mRNA, | 1e-012 | |
| 1938 | U50156 | Human DNA segment containing CA repeat at locus | 5e-013 | |
| 1939 | X65708 | H. sapiens RRM1 gene for ribonucleoside diphosphate | 5e-013 | |
| | | reductase M1 subunit | | |
| 1940 | M61107 | Human p22-phox (CYBA) gene, exons 3 and 4. | 5e-013 | |
| 1941 | AC001502 | Homo sapiens (subclone 2_c7 from P1 H43) DNA sequence | 4e-013 | |
| 1942 | X69951 | H. sapiens gene for casein kinase II alpha subunit > subunit | 4e-013 | |
| | | alpha [human, Genomic, 18862 nt] | | |
| 1943 | AC002252 | Homo sapiens (subclone 1_g7 from BAC H76) DNA | 4e-013 | |
| 1944 | Z74029 | Caenorhabditis elegans cosmid C45B11, complete sequence | 4e-013 | |
| | | [Caenorhabditis elegans] | | |
| 1945 | U90544 | Human sodium phosphate transporter (NPT3) mRNA, | 1e-013 | |
| 1946 | L77032 | Homo sapiens (subclone 3_e5 from P1 H16) DNA sequence. | 2e-013 | |
| 1947 | X55367 | Human alpha-satellite DNA from clone pTRA-2 | 2e-013 | |
| 1948 | U40369 | Human spermidine/spermine N1-acetyltransferase | 2e-013 | |
| 1949 | Z25749 | H. sapiens gene for ribosomal protein S7 | 2e-013 | |
| 1950 | M96838 | Human proteinase 3 gene, exons 2 and 3. | 2e-013 | |
| 1951 | Z73116 | S. cerevisiae chromosome XII reading frame ORF YLL011w | 2e-013 | |
| 1952 | U90544 | Human sodium phosphate transporter (NPT3) mRNA, | 2e-013 | |
| 1953 | U90544 | Human sodium phosphate transporter (NPT3) mRNA, | 2e-013 | |
| 1954 | AC001016 | Homo sapiens (subclone 2_f8 from P1 H43) DNA sequence | 1e-013 | |
| 1955 | D00596 | Homo sapiens gene for thymidylate synthase, exons 1, 2, 3, | 1e-013 | |
| | | 4, 5, 6, 7, complete cds | | |
| 1956 | X80240 | H. sapiens endogenous retrovirus HERV-KC4 DNA | 1e-013 | |
| 1957 | M86181 | Human prosaposin (PSAP) gene. | 5e-014 | |
| 1958 | V00710 | Human mitochondrial genes for several tRNAs (Phe, Val, | 5e-014 | |
| | | Leu) and 12S and 16S ribosomal RNAs | | |
| 1959 | Z62151 | H. sapiens CpG island DNA genomic Mse1 fragment, clone | 5e-014 | |
| | | 64c7, forward read cpg64c7.ft1a | | |
| 1960 | NM_002187.1 | Homo sapiens interleukin 12B (natural killer cell stimuiatory | 5e-014 | |
| | | factor 2, cytotoxic lymphocyte maturation factor 2, p40) | | |
| | | (IL12B) mRNA > :: gb\|M65290\|HUMNKSFP40 Human | | |
| | | natural killer cell stimulatory factor (NKSF) mRNA, | | |
| 1961 | M18680 | Homo sapiens 5S rRNA pseudogene. | 5e-014 | |
| 1962 | X06274 | Human endogenous retrovirus-like sequence (LTR ERS-P1- | 5e-014 | |
| | | 1) 3′-LTR region | | |
| 1963 | D00835 | Human immunodeficiency virus type 2 proviral DNA, | 5e-014 | |
| | | complete genome | | |
| 1964 | V00581 | Human Alu repetitive located near the insulin gene > | 5e-014 | |
| | | :: gb\|J00268\|HUMINS02 Human insulin gene: repetitive | | |
| | | sequence in 3′ flank. | | |
| 1965 | Z95437 | Human DNA sequence from cosmid A1 on chromosome 6 | 5e-014 | |
| | | contains ESTs. HERV like retroviral sequence | | |
| 1966 | AB001051 | Dugesia japonica mRNA for ADP-ribosylation factor, | 5e-014 | |
| 1967 | AB001051 | Dugesia japonica mRNA for ADP-ribosylation factor, | 5e-014 | |
| 1968 | M59709 | Human carcinoembryonic antigen (CEA) gene, exon 10. | 2e-014 | |
| 1969 | X91233 | H. sapiens IL15 gene | 2e-014 | |
| 1970 | X12718 | Human Retrovirus mRNA for LTR (clone cPB-3) | 2e-014 | |
| 1971 | V00531 | Human interferon genes LeIF-L and LeIF-J and pseudogene | 1e-014 | |
| | | LeIF-M with intergenic regions. These genes are located on | | |
| 1972 | M14292 | Human L1Heg repetitive element from the intergenic region | 1e-014 | |
| | | of the epsilon and G-gamma globin genes. | | |
| 1973 | X56998 | Human UbA52 adrenal mRNA for ubiquitin-52 amino acid | 1e-014 | |
| | | fusion protein | | |
| 1974 | S74906 | E1 beta = pyruvate dehydrogenase beta {promoter} | 8e-015 | |
| 1975 | M18680 | Homo sapiens 5S rRNA pseudogene. | 6e-015 | |
| 1976 | L49046 | Homo sapiens (subclone 2_h3 from P1 H25) DNA sequence. | 6e-015 | |
| 1977 | M18680 | Homo sapiens 5S rRNA pseudogene. | 6e-015 | |
| 1978 | U93037 | Homo sapiens elastin gene, exons 5-27 and alternatively | 6e-015 | |
| | | spliced products, partial cds | | |
| 1979 | M76741 | Homo sapiens biliary glycoprotein (BGP) gene, partial cds. | 6e-015 | |
| 1980 | U72787 | Homo sapiens cosmid clone U163C11 from Xp22.114 22.2, | 6e-015 | |
| | | complete sequence [Homo sapiens] | | |
| 1981 | AB000931 | Homo sapiens FUT2 gene, intron 1, complete sequence | 6e-015 | |
| 1982 | M83137 | Human scaffold-attached region (SAR) DNA. | 6e-015 | |
| 1983 | M18680 | Homo sapiens 5S rRNA pseudogene. | 5e-015 | |
| 1984 | Z63454 | H. sapiens CpG island DNA genomic Mse1 fragment, clone | 5e-015 | |
| | | 84d2, reverse read cpg84d2.rt1a | | |
| 1985 | Z63454 | H. sapiens CpG island DNA genomic Mse1 fragment, clone | 5e-015 | |
| | | 84d2, reverse read cpg84d2.rt1a | | |
| 1986 | X97489 | H. sapiens PIT1/GHF1 gene silencer region | 5e-015 | |
| 1987 | X55367 | Human alpha-satellite DNA from clone pTRA-2 | 2e-015 | |
| 1988 | Z22795 | H. sapiens microsatellite repeat | 2e-015 | |
| 1989 | D38112 | Human mitochondrial DNA, complete sequence | 2e-015 | |
| 1990 | X69951 | H. sapiens gene for casein kinase II alpha subunit subunit | 2e-015 | |
| | | alpha [human, Genomic, 18862 nt] | | |
| 1991 | M59250 | Homo sapiens cytochrome c oxidase subunit Vb | 2e-015 | |
| 1992 | X15965 | Rabbit DNA fbr L1Oc5 repeat | 2e-015 | |
| 1993 | X70052 | S. cerevisiae sof1 gene | 2e-015 | |
| 1994 | M86667 | H. sapiens NAP (nucleosome assembly protein) mRNA, | 1e-015 | |
| 1995 | X78212 | H. sapiens diamine oxidase gene | 7e-016 | |
| 1996 | U21928 | Human fructose-1,6-biphosphatase (FBP1) gene, exon 4 | 7e-016 | |
| 1997 | AB001051 | Dugesia japonica mRNA for ADP-ribosylation factor, | 7e-016 | |
| 1998 | AC002181 | Homo sapiens (subclone 2_a12 from BAC H111) DNA | 6e-016 | |
| 1999 | X91413 | H. sapiens DNA for X-linked dinucleotide repeat | 6e-016 | |
| | | polimorphism (clone YCA3CL149) | | |
| 2000 | U65487 | Human ribosomal RNA upstream binding transcription | 6e-016 | |
| | | factor (UBTF) gene, partial cds | | |
| 2001 | Z69920 | Human DNA sequence from cosmid 91K3, Huntington's | 6e-016 | |
| | | Disease Region, chromosome 4p16.3 | | |
| 2002 | U92818 | Homo sapiens c33.28 unnamed HERV-H protein mRNA, | 6e-016 | |
| 2003 | J03799 | Human colin carcinoma laminin-binding protein mRNA, | 3e-016 | |
| 2004 | Z68885 | Human DNA sequence from cosmid L21F12B, Huntington's | 2e-016 | |
| | | Disease Region, chromosome 4p16.3, contains EST | | |
| 2005 | U75285 | Homo sapiens apoptosis inhibitor survivin gene,complete | 2e-016 | |
| 2006 | M23442 | Human interleukin 4 (IL-4) gene, complete cds. | 2e-016 | |
| 2007 | U18270 | Human thymopoietin (TMPO) gene, exons 4 and 5, and | 2e-016 | |
| | | complete cds for thymopoietin alpha | | |
| 2008 | AD000833 | Homo sapiens DNA from chromosome 19-cosmid f19399 | 2e-016 | |
| | | (˜17 kb EcoRI restriction fragment) | | |
| 2009 | AD000833 | Homo sapiens DNA from chromosome 19-cosmid f19399 | 2e-016 | |
| | | (˜17 kb EcoRI restriction fragment) | | |
| 2010 | Y14823 | Drosophila melanogaster SURF-4 gene and gene encoding | 2e-016 | |
| | | seryl-tRNA synthetase | | |
| 2011 | U67167 | Homo sapiens intestinal mucin (MUC2) gene, promoter | 8e-017 | |
| | | region and partial cds | | |
| 2012 | Z96177 | H. sapiens telomeric DNA sequence, clone 10QTEL040, | 7e-017 | |
| | | 10QTELOO040.seq | | |
| 2013 | AF003533 | Homo sapiens cytosolic phagocyte oxidase protein | 7e-017 | |
| | | (p47phox) gene, promoter region and partial cds | | |
| 2014 | NM_000151.1 | Homo sapiens glucose-6-phosphatase, catalytic glucose-6- | 7e-017 | |
| | | phosphatase mRNA, complete cds. > :: gb\|I15157\|I15157 | | |
| | | Sequence 1 from U.S. Pat. No. 5460942 | | |
| 2015 | X94912 | H. sapiens Pr22 gene | 7e-017 | |
| 2016 | U10580 | Human junction sequence from chimeric/rearranged YAC | 7e-017 | |
| | | yRM2252, chromosome 11p14. | | |
| 2017 | M22970 | Human pancreatic phospholipase A-2 (PLA-2) gene, exons 1 | 7e-017 | |
| 2018 | X02152 | Human mRNA for lactate dehydrogenase-A (LDH-A, EC | 6e-017 | |
| 2019 | Human DNA sequence from cosmid L118D5, Huntington's | 6e-017 | | |
| | | Disease Region, chromosome 4p16.3 | | |
| 2020 | M64804 | Human microsatellite DNA repeat region DNA. > :: | 3e-017 | |
| | | gb\|I31115\|I31115 Sequence 27 from U.S. Pat. No. 5582979 | | |
| 2021 | Z92910 | H. sapiens HFE (HLA-H) gene | 3e-017 | |
| 2022 | X74984 | H. sapiens 5′ flanking region of CD14 gene | 2e-017 | |
| 2023 | U05333 | Mus musculus co-chaperomn ‘cofactor A’ mRNA, complete | 2e-017 | |
| 2024 | U48485 | Human skeletal muscle ryanodine receptor gene | 2e-017 | |
| 2025 | X97869 | H. sapiens gene encoding La autoantigen | 2e-017 | |
| 2026 | X17579 | Human specific H55 DNA | 2e-017 | |
| 2027 | U36445 | Bos taurus calcium-activated chloride channel mRNA, | 2e-017 | |
| 2028 | L06845 | Human cysteinyl-tRNA synthetase mRNA, partial cds. | 2e-017 | |
| 2029 | X93334 | H. sapiens mitochondrial DNA, complete genome | 8e-018 | |
| 2030 | X62996 | H. sapiens mitochondrial genome (consensus sequence) | 8e-018 | |
| 2O31 | M98479 | Human transglutaminase mRNA, 3′ untranslated region. | 8e-018 | |
| 2032 | L42568 | Homo sapiens (clone 1SW11-1) non-gastric H, K-ATPase | 8e-018 | |
| | | (ATP1AL1) gene, exons 15-17 | | |
| 2033 | S52659 | lactoferrin {5′ region, promoter} [human, placenta, | 8e-018 | |
| 2034 | U66707 | Rattus norvegicus densin-180 mRNA, complete cds | 8e-018 | |
| 2035 | X76683 | Plasmid vector pHM2 betalactamase gene | 3e-018 | |
| 2036 | U66707 | Rattus norvegicus densin-180 mRNA, complete cds | 3e-018 | |
| 2037 | D17201 | Human HepG2 3′ region MboI cDNA, clone hmd3d04m3 | 3e-018 | |
| 2038 | Z24281 | H. sapiens (D12S355) DNA segment containing | 2e-018 | |
| 2039 | AC001443 | Homo sapiens (subclone 2_f10 from BAC 2913 | 2e-018 | |
| 2040 | U73522 | Homo sapiens AMSH mRNA, complete cds | 1e-018 | |
| 2041 | L77040 | Homo sapiens (subclone 8_c11 from P1 H22) DNA | 9e-019 | |
| 2042 | X93496 | H. sapiens TRAP gene, 5′ flanking region | 9e-019 | |
| 2043 | X54816 | Human gene for alpha-1-microglobulin-bikunin, exons 1-5 | 9e-019 | |
| | | (encoding alpha-1-microglobulin, N-terminus.) | | |
| 2044 | L35240 | Human enigma gene, complete cds | 8e-019 | |
| 2045 | Z46940 | H. sapiens PRM1 gene, PRM2 gene and TNP2 gene | 8e-019 | |
| 2046 | U60801.1 | Human poly(A)-binding protein (PABP) processed | 8e-019 | |
| | | pseudogene2, complete cds | | |
| 2047 | NM_002484.1 | Homo sapiens nucleotide binding protein 1 Human | 4e-019 | |
| | | nucleotide-binding protein mRNA, complete cds. | | |
| 2048 | X76683 | Plasmid vector pHM2 betalactamase gene | 3e-019 | |
| 2049 | U40369 | Human spermidine/spermine N1-acetyltransferase | 3e-019 | |
| 2050 | D13624 | Human Wilms tumor gene encoding WT1 protein, exon 6 | 3e-019 | |
| 2051 | D13624 | Human Wilms tumor gene encoding WT1 protein, exon 6 | 3e-019 | |
| 2052 | D86566 | Human DNA for NOTCH4, partial cds | 3e-019 | |
| 2053 | U72787 | Homo sapiens cosmid clone U163C11 from Xp22.1-22.2, | 3e-019 | |
| | | complete sequence [Homo sapiens] | | |
| 2054 | U86759 | Human netrin-2 like protein (NTN21) mRNA, complete cds | 3e-019 | |
| 2055 | X78454 | X. laevis AB21 for RPD3 homologue | 3e-019 | |
| 2056 | NM_000969.1 | Homo sapiens ribosomal protein L5 (RPL5) mRNA | 1e-019 | |
| 2057 | U49869 | Human ubiquitin gene, complete cds | 1e-019 | |
| 2058 | X78901 | H. sapiens (lambda63) DNA of apolipoprotein cluster | 9e-020 | |
| 2059 | U32515 | Human putative tumor suppressor (MXI1) gene, exons 4, 5, | 9e-020 | |
| | | and 6, and complete cds | | |
| 2060 | D87717 | Human mRNA for KIAA0013 gene, complete cds | 5e-020 | |
| 2061 | Z68758 | Human DNA sequence from cosmid cN85E10 on | 3e-020 | |
| | | chromosome 22q11.2-qter | | |
| 2062 | Z30584 | R. norvegicus (wistar) mRNA for ZG-16p | 3e-020 | |
| 2063 | Z68758 | Human DNA sequence from cosmid cN85E10 on | 3e-020 | |
| | | chromosome 22q11.2-qter | | |
| 2064 | M58600 | Human heparin cofactor II (HCF2) gene, exons 1 through 5. | 3e-020 | |
| 2065 | U93037 | Homo sapiens elastin gene, exons 5-27 and alternatively | 3e-020 | |
| | | spliced products, partial cds | | |
| 2066 | L38951 | Homo sapiens importin beta subunit mRNA, complete cds | 2e-020 | |
| 2067 | D26141 | Human NF1 gene homologue | 1e-020 | |
| 2068 | M18796 | Orangutan beta- and delta-globin gene intergenic region with | 1e-020 | |
| | | 2 Alu repeats. | | |
| 2069 | Z47046 | Human cosmid QLL2C9 from Xq28 | 1e-020 | |
| 2070 | AC001443 | Homo sapiens pl (subclone 2_f10 from BAC 2913 | 1e-020 | |
| 2071 | X15965 | Rabbit DNA for L1Oc5 repeat | 1e-020 | |
| 2072 | U86453 | Human phosphatidylinositol 3-kinase catalytic subunit | 6e-021 | |
| | | p110delta mRNA, complete cds | | |
| 2073 | M90058 | Human serglycin gene, exons 1, 2, and 3. | 4e-021 | |
| 2074 | L81932 | Homo sapiens (subclone 9_h2 from P1 H21) DNA sequence | 4e-021 | |
| 2075 | X68258 | Bicistronic transcription units (pSBC-2) | 3e-021 | |
| 2076 | L78777 | Homo sapiens (subclone 2_b8 from P1 H49) DNA sequence | 3e-021 | |
| 2077 | J01415 | Human mitochondrion, complete genome | 1e-021 | |
| 2078 | L43411 | Homo sapiens (subclone 5_g5 from P1 H25) DNA sequence. | 1e-021 | |
| 2079 | NM_000967.1 | Homo sapiens ribosomal protein L3 (RPL3) mRNA protein | 1e-021 | |
| 2080 | Z81014 | Human DNA sequence from cosmid U65A4, between | 1e-021 | |
| | | markers DXS366 and DXS87 on chromosome X * | | |
| 2081 | U93037 | Homo sapiens elastin gene, exons 5-27 and altematively | 1e-021 | |
| | | spliced products, partial cds | | |
| 2082 | X78454 | X. laevis AB21 mRNA for RPD3 homologue | 1e-021 | |
| 2083 | X82825 | A. thaliana PRL1 mRNA | 1e-021 | |
| 2084 | J01415 | Human mitochondrion, complete genome | 4e-022 | |
| 2085 | Z68885 | DNA sequence from cosmid L21F12B, Huntington's | 4e-022 | |
| | | Disease Region, chromosome 4p16.3, contains EST | | |
| 2086 | L81690 | Homo sapiens (subclone 1_c2 from P1 H56) DNA sequence | 4e-022 | |
| 2087 | Z7046 | Human cosmid QLL2C9 from Xq28 | 4e-022 | |
| 2088 | Z73360 | Human DNA sequence from cosmid 92M18, BRCA2 gene | 1e-022 | |
| | | region chromosome 13q12-13 | | |
| 2089 | L78776 | Homo sapiens (subclone 2_a7 from P1 H49) DNA sequence | 1e-022 | |
| 2090 | Z96210 | H. sapiens telomeric DNA sequence, clone 12PTEL057, read | 5e-023 | |
| | | 12PTELOO057.seq | | |
| 2091 | Z50751 | H. sapiens mRNA for B4B | 4e-023 | |
| 2092 | U14567 | ***ALU WARNING: Human Alu-J subfamily consensus | 4e-023 | |
| 2093 | L35657 | Homo sapiens (subclone H85 a_a10 from P1 35 H5 C8) DNA | 4e-023 | |
| 2094 | Z81315 | Human DNA sequence from fosmid F62D4 on chromosome | 4e-023 | |
| | | 22q12-qter > :: emb\|Z81316\|HSF62D4A Human DNA | | |
| | | sequence from fosmid F62D4 on chromosome 22, complete | | |
| 2095 | U93237 | Human menin (MEN 1) gene, complete cds | 1e-023 | |
| 2096 | M16660 | Human 90-kDa heat-shock protein gene, cDNA, complete | 1e-023 | |
| 2097 | Z93943 | Human DNA sequence from cosmid U235H3 on | 6e-024 | |
| 2098 | M84334 | Macacca mulatta hnRNP A1-gamma isoform mRNA, | 5e-024 | |
| 2099 | Z73360 | Human DNA sequence from casmid 92M18, BRCA2 gene | 5e-024 | |
| | | region chromosome 13q12-13 | | |
| 2100 | L32754 | Homo sapiens Ig-associated signalling molecule | 5e-024 | |
| 2101 | AC002252 | Homo sapiens (subclone 1_g7 from BAG H76) DNA | 5e-024 | |
| 2102 | NM_000982.1 | Homo sapiens ribosomal protein L21 (RPL21) mRNA > :: | 5e-024 | |
| | | gb\|U25789\|HSU25789 Human ribosomal protein L21 | | |
| | | mRNA, complete cds. | | |
| 2103 | NM_000397.1 | Homo sapiens cytochrome b-245, beta polypeptide encoding | 5e-024 | |
| | | mitochondrial protein, mRNA > :: emb\|X04011\|HSXCGD | | |
| | | Human mRNA of X-CGD gene involved in chronic | | |
| | | granulomatous disease located on chromosome X | | |
| 2104 | L19086 | Human LINE1 (L1.3) repetitive element DNA sequence. 4e-024 | | |
| 2105 | U88531 | Bos taurus phosphatidylinositol 4-kinase mRNA, complete | 2e-024 | |
| 2106 | D38112 | Human mitochondrial DNA, complete sequence | 2e-024 | |
| 2107 | NM_003011.1 | Homo sapiens SET translocation (myeloid leukemia- | 2e-024 | |
| | | associated) (SET) mRNA > :: gb\|M93651\|HUMSET Human | | |
| 2108 | M98512 | Human NFG genomic fragment. | 2e-024 | |
| 2109 | NM_001019.1 | Homo sapiens ribosomal protein S15a for ribosomal protein | 2e-024 | |
| 2110 | X70991 | H. sapiens MADER mRNA | 2e-024 | |
| 2111 | X14445 | Human int-2 proto-oncogene | 2e-024 | |
| 2112 | AC001174 | Homo sapiens (subclone 1_e2 from BAG H94) DNA | 2e-024 | |
| 2113 | D86566 | Human DNA for NOTCH4, partial cds | 2e-024 | |
| 2114 | Z78885 | H. sapiens flow-sorted chromosome 6 HindIII fragment, 1e-024 | | |
| 2115 | Z82272 | Human endogenous retrovirus env mRNA | 1e-024 | |
| 2116 | Z96167 | H. sapiens telomeric DNA sequence, clone 10QTEL017, | 6e-025 | |
| | | read 10QTELOO017.seq | | |
| 2117 | L38951 | Homo sapiens importin beta subunit mRNA, complete cds | 6e-025 | |
| 2118 | X53575 | Yeast RPL7 gene for ribosmal protein L7 | 6e-025 | |
| 2119 | L77040 | Homo sapiens (subclone 8_c11 from P1 H22) DNA | 5e-025 | |
| 2120 | Z23957 | H. sapiens (D2S336) DNA segment containing (CA) repeat; | 5e-025 | |
| | | clone AFM275yf5; single read | | |
| 2121 | X14445 | Human int-2 proto-oncogene | 5e-025 | |
| 2122 | L39062 | Homo sapiens interleukin 9 receptor IL9R pseudogene, | 2e-025 | |
| 2123 | Z84723 | Human DNA sequence from phage LAW2 from a contig | 2e-025 | |
| | | from the tip of the short arm of chromosome 16, spanning | | |
| | | 2 MB of 16p13.3 Contains Interleukin 9 receptor pseudogene | | |
| 2124 | AC001174 | Homo sapiens (subclone 1_e2 from BAC H94) DNA | 2e-025 | |
| 2125 | NM_001423.1 | Homo sapiens epithelial membrane protein 1 Progression | 2e-025 | |
| | | Associated Protein | | |
| 2126 | X14445 | Human int-2 proto-oncogene | 2e-025 | |
| 2127 | M68841 | Human L1 repetitive sequence with a region homologous to | 2e-025 | |
| | | a mouse ORF. | | |
| 2128 | X97489 | H. sapiens PIT1/GHF1 gene silencer region | 2e-025 | |
| 2129 | X73501 | H. sapiens gene for cytokeratin 20 | 1e-025 | |
| 2130 | U8363 | Mus musculus transcriptional activator alpha-NAG | 1e-025 | |
| 2131 | X01037 | Human 7SL RNA sequence | 7e-026 | |
| 2132 | M22485 | Human ring chromosome 21 [r(21)] breakpoint DNA. | 2e-026 | |
| 2133 | K03429 | Ape (chlmpanzee) 28S ribosomal RNA gene. | 2e-026 | |
| 2134 | AF053644 | Homo sapiens cellular apoptosis susceptibility protein | 9e-027 | |
| | | (CSE1) gene, exon 2 | | |
| 2135 | L06900 | Human dystrophin gene, intron 1 containing pseudo exon. | 7e-027 | |
| 2136 | U18271 | Human thymopoietin (TMPO) gene, partial exon 6, | 7e-027 | |
| | | complete exon 7, partial exon 8, and partial cds for | | |
| 2137 | X05323 | Human MRC OX-2 gene signal sequence | 7e-027 | |
| 2138 | M99065 | Rat core histone (MacroH2A.1) mRNA, complete cds. | 7e-027 | |
| 2139 | X01037 | Human 7SL RNA sequence | 6e-027 | |
| 2140 | M22485 | Human ring chromosome 21 [r(21)] breakpoint DNA. | 2e-027 | |
| 2141 | D50494 | Mouse mRNA for murine RCK, complete cds | 2e-027 | |
| 2142 | M14292 | Human L1Heg repetitive element from the intergenic region | 2e-027 | |
| | | of the epsilon and G-gamma globin genes. | | |
| 2143 | U72787 | Homo sapiens cosmid clone U163C11 from Xp22.1-22.2, | 7e-028 | |
| | | complete sequence [Homo sapiens ] | | |
| 2144 | NM_001030.1 | Homo sapiens ribosomal protein S27 gb\|L19739\|HUMMPSI | 7e-028 | |
| | | Homo sapiens metallopanstimul (MPS1) mRNA, complete | | |
| | | cds. > :: gb\|I60224\|I60224 Sequence 1 from patent US | | |
| 2145 | AC002186 | Homo sapiens (subclone 1_f12 from P1 H115) DNA | 7e-028 | |
| 2146 | M22485 | Human ring chromosome 21 [r(21)] breakpoint DNA. | 3e-028 | |
| 2147 | Z95437 | Human DNA sequence from cosmid A1 on chromosome 6 | 2e-028 | |
| | | contains ESTs. HERV like retroviral sequence | | |
| 2148 | NM_001025.1 | Homo sapiens ribosomal protein S23 (RPS23) mRNA > :: | 2e-028 | |
| | | dbj\|D14530\|HUMRSPT Human homolog of yeast ribosomal | | |
| | | protein S28, complete cds | | |
| 2149 | Z95437 | Human DNA sequence from cosmid A1 on chromosome 6 | 2e-028 | |
| | | contains ESTs. HERV like retroviral sequence | | |
| 2150 | Z62151 | H. sapiens CpG island DNA genomic Mse1 fragment, clone | 1e-028 | |
| | | 64c7, forward read cpg64c7.ft1a | | |
| 2151 | Z62151 | H. sapiens CpG island DNA genomic Mse1 fragment, clone | 8e-029 | |
| | | 64c7, forward read cpg64c7.ft1a | | |
| 2152 | X62996 | H. sapiens mitochondrial genome (consensus sequence) | 8e-029 | |
| 2153 | Z62151 | H. sapiens CpG island DNA genomic Mse1 fragment, clone | 8e-029 | |
| | | 64c7,forward read cpg64c7.ft1a | | |
| 2154 | AC001518 | Homo sapiens (subclone 2_b4 from P1 H49) DNA sequence | 8e-029 | |
| 2155 | X56932 | H. sapiens mRNA for 23 kD highly basic protein | 8e-029 | |
| 2156 | U93563 | Human L1 element L1.6 putative p150 gene, complete cds | 8e-029 | |
| 2157 | Z96282 | H. sapiens telomeric DNA sequence, clone 13QTEL058, | 8e-029 | |
| | | read 13QTELOO05.seq | | |
| 2158 | NM_002892.1 | Homo sapiens retinoblastoma-binding protein 1 (RBBP1) | 7e-029 | |
| | | mRNA > :: gb\|S66427\|S66427 RBP1 = retinoblastoma | | |
| | | binding protein 1 [human, Nalm-6 pre-B cell leukemia, | | |
| 2159 | M12855 | Human endogenous retrovirus DNA downstream of 5′ LTR, | 3e-029 | |
| | | clone HERV-K22. | | |
| 2160 | X86012 | Human DNA sequence from intron 22 of the factor VIII | 3e-029 | |
| | | gene, Xq28. Contains the end of a 9.5 kb repeated region, | | |
| | | int22h-1, involved in many cases of haemophilia | | |
| 2161 | L81840 | Homo sapiens (subclone 1_f8 from P1 H43) DNA sequence | 3e-029 | |
| 2162 | Z11711 | H. sapiens gene for alpha-2 macroglobulin, exon 1 | 3e-029 | |
| 2163 | X61453 | M. musculus mRNA for H19 clone | 3e-029 | |
| 2164 | NM_003389.1 | Homo sapiens WD repeat domain 2 (WDR2) mRNA > | 1e-029 | |
| 2165 | L81840 | Homo sapiens (subclone 1_fB from P1 H43) DNA sequence | 9e-030 | |
| 2166 | L05173 | Homo sapiens chromosome 21 DNA fragment with Alu, L1 | 8e-030 | |
| | | and O repetitive elements. | | |
| 2167 | M12855 | Human endogenous retrovirus DNA downstream of 5′ LTR, | 3e-030 | |
| | | clone HERV-K22. | | |
| 2168 | M12855 | Human endogenous retrovirus DNA downstream of 5′ LTR, | 3e-030 | |
| | | clone HERV-K22. | | |
| 2169 | J05211 | Human desmoplakin mRNA, 3′ end. | 3e-030 | |
| 2170 | Z96167 | H. sapiens telomeric DNA sequence, clone 10QTEL017, | 3e-030 | |
| | | read 10QTELOO017.seq | | |
| 2171 | M12855 | Human endogenous retrovirus DNA downstream of 5′ LTR, | 3e-030 | |
| | | clone HERV-K22. | | |
| 2172 | AC002186 | Homo sapiens (subclone 1_f12 from P1 H115) DNA | 3e-030 | |
| 2173 | L35657 | Homo sapiens (subclone H8 5_a10 from P1 35 H5 C8) DNA | 3e-030 | |
| 2174 | L35657 | Homo sapiens (subclone H8 5_a10 from P1 35 H5 C8) DNA | 3e-030 | |
| 2175 | L39061 | Homo sapiens transcription factor SL1 mRNA, partial cds. | 3e-030 | |
| 2176 | AF010313 | Homo sapiens Pig8 (PIG8) mRNA, complete cds | 1e-030 | |
| 2177 | AC001443 | Homo sapiens (subclone 2_f10 from BAC 2913 | 1e-030 | |
| 2178 | Z62151 | H. sapiens CpG island DNA genomic Mse1 fragment, clone | 3e-031 | |
| | | 64c7, forward read cpg64c7.ft1a | | |
| 2179 | AF010312 | Homo sapiens Pig7 (PIG7) mRNA, complete cds | 1e-031 | |
| 2180 | U77085 | Human epithelial membrane protein (CL-20) mRNA, | 1e-031 | |
| 2181 | D29805 Human mRNA for beta-1,4-galactosyltransferase, complete | 1e-031 | | |
| 2182 | Z95437 | Human DNA sequence from cosmid A1 on chromosome 6 | 1e-031 | |
| | | contains ESTs. HERV like retroviral sequence | | |
| 2183 | X62996 | H. sapiens mitochondrial genome (consensus sequence) | 1e-031 | |
| 2184 | U80409 | Lactococcus lactis cremoris glucose inhibited division | 4e-032 | |
| | | protein homolog GidA (gidA) gene, partial cds | | |
| 2185 | X00525 | Mouse 28S ribosomal RNA | 1e-032 | |
| 2186 | U35032 | Human endogenous retrovirus clone c5.11, HERV-H | 1e-032 | |
| | | multiply spliced subgenomic leader, protease and integrase | | |
| 2187 | Z75894 | Human DNA sequence from cosmid U61F10, between | 1e-032 | |
| | | markers DXS366 and DXS87 on chromosome X contains | | |
| 2188 | U22055 | Human 100 kDa coactivator mRNA, complete cds. | 1e-032 | |
| 2189 | M98509 | Human NFB genomic fragment. | 5e-033 | |
| 2190 | D38112 | Human mitochondrial DNA, complete sequence | 2e-033 | |
| 2191 | Z69364 | Human DNA sequence from cosmid L96F8, Huntington's | 5e-034 | |
| | | Disease Region, chromosome 4p16.3 contains EST and | | |
| | | cDNA > :: emb\|Z69365\|HSL96F8A Human DNA sequence | | |
| | | from cosmid L96F8, Huntington's Disease Region, | | |
| | | chromosome 4p16.3 contains EST and cDNA | | |
| 2192 | U59695 | Human apurinic/apyrimidinic endonuclease (HAP1) gene, 5′ | 5e-034 | |
| | | upstream region | | |
| 2193 | L35657 | Homo sapiens (subclone H8 5_a10 from P1 35 H5 C8) DNA | 2e-034 | |
| 2194 | L78778 | Homo sapiens (subclone 2_e10 from P1 H49) DNA | 2e-034 | |
| 2195 | V00662 | H. sapiens mitochondrial genome | 5e-035 | |
| 2196 | M30629 | Human pregnancy-specific glycoprotein beta-1 | 5e-035 | |
| 2197 | NM_003134.1 | Homo sapiens signal recognition particle 14 kD (homologous | 5e-035 | |
| | | Alu RNA-binding protein) (SRP14) mRNA > recognition | | |
| 2198 | U93572 | Human L1 element L1.25 p40 and putative p150 genes, | 2e-035 | |
| 2199 | Z96176 | H. sapiens telomeric DNA sequence, clone 10QTEL038, | 6e-036 | |
| | | read 10QTELOO038.seq | | |
| 2200 | V00710 | Human mitochondrial genes for several tRNAs (Phe, Val, | 6e-036 | |
| | | Leu) and 12S and 16S ribosomal RNAs | | |
| 2201 | Z78715 | H. sapiens flow-sorted chromosome 6 HindIII fragment, | 5e-036 | |
| 2202 | U93574 | Human L1 element L1.39 p40 and putative p150 genes, | 2e-036 | |
| 2203 | S63912 | D10S102 = FBRNP [human, fetal brain, mRNA, 3043 nt] | 1e-036 | |
| 2204 | AB011137 | Homo sapiens mRNA for KIAA0565 protein, complete cds | 1e-036 | |
| 2205 | Z55370 | H. sapiens CpG island DNA genomic Mse1 fragment, clone | 7e-037 | |
| | | 37a7, forward read cpg37a7.ft1a | | |
| 2206 | L36720 | Homo sapiens bystin mRNA, complete cds | 7e-037 | |
| 2207 | S45936 | HTS1 = HeLa tumor suppressor gene [human, revertant clone | 7e-037 | |
| | | F2, mRNA Partial, 2687 nt] | | |
| 2208 | U36755 | Human thrombin receptor (F2R) gene, 5′ region and partial | 6e-037 | |
| 2209 | D50694 | Rattus norvegicus mRNA for proteasomal ATPase | 3e-037 | |
| 2210 | AB001325 | Human AQP3 gene for aquaporine 3 (water channel), partail | 2e-037 | |
| 2211 | L35657 | Homo sapiens (subclone H8 5_a10 from P1 35 H5 C8) DNA | 2e-037 | |
| 2212 | U36445 | Bos taurus calcium-activated chloride channel mRNA, | 2e-037 | |
| 2213 | X66292 | P. pygmaeus (OX3910-11) alphoid repetitive DNA | 7e-038 | |
| 2214 | D38112 | Human mitochondrial DNA, complete sequence | 6e-038 | |
| 2215 | Z16571 | H. sapiens (D12S80) DNA segment containing (CA) repeat; | 6e-038 | |
| | | clone AFM102xd6; single read | | |
| 2216 | D50561 | Human DNA, replication enhancing element (REE1) | 3e-038 | |
| 2217 | X66292 | P. pygmaeus (OX3910-11) alphoid repetitive DNA | 8e-039 | |
| 2218 | Z73360 | Human DNA sequence from cosmid 92M18, BRCA2 gene | 7e-039 | |
| | | region chromosome 13q12-13 | | |
| 2219 | M25718 | Human rDNA and 4 Alu repeats. | 4e-039 | |
| 2220 | NM_000397.1 | Homo sapiens cytochrome b-245, beta polypeptide encoding | 3e-039 | |
| | | mitochondrial protein, mRNA > :: emb\|X04011\|HSXCGD | | |
| | | Human mRNA of X-CGD gene involved in chronic | | |
| | | granulomatous disease located on chromomosome X | | |
| 2221 | M13073 | Human metallothionein I processed pseudogene, complete | 2e-039 | |
| 2222 | Z78715 | H. sapiens flow-sorted chromosome 6 HindIII fragment, | 3e-040 | |
| 2223 | U72787 | Homo sapiens cosmid clone U163C11 from Xp22.1-22.2, | 3e-040 | |
| | | complete sequence [Homo sapiens] | | |
| 2224 | Z12962 | H. sapiens mRNA for homologue to yeast ribosomal protein | 3e-040 | |
| 2225 | Z78715 | H. sapiens flow-sorted chromosome 6 HindIII fragment, | 9e-041 | |
| 2226 | AC001443 | Homo sapiens (subclone 2_f10 from BAC 2913 | 4e-041 | |
| 2227 | Z73360 | Human DNA sequence from cosmid 92M18, BRCA2 gene | 3e-041 | |
| | | region chromosome 13q12-13 | | |
| 2228 | S72304 | rah = ras-related homolog [mice, HT4 neural cell line, | 3e-041 | |
| 2229 | AC001443 | Homo sapiens (subclone 2_f10 from BAG 2913 | 1e-041 | |
| 2230 | U49974 | Human mariner2 transposable element, complete consensus | 1e-041 | |
| 2231 | L26507 | Mouse myocyte nuclear factor (MNF) mRNA, complete cds. | 1e-041 | |
| 2232 | U08191 | Human R kappa B mRNA, complete cds. | 1e-042 | |
| 2233 | D11078 | Homo sapiens RGH2 gene, retrovirus-like element | 1e-042 | |
| 2234 | U83908 | Human nuclear antigen H731 mRNA, complete cds | 1e-042 | |
| 2235 | X63209 | B. taurus CI-ASHI mRNA for ubiquinone oxidoreductase | 1e-042 | |
| 2236 | NM_002743.1 | Homo sapiens protein kinase C substrate 80K-H (PRKCSH) | 1e-042 | |
| | | mRNA > :: gb\|J03075\|HUMG19P1A Human 80K-H protein | | |
| | | (kinase C substrate) mRNA, complete cds. | | |
| 2237 | L78777 | Homo sapiens (subclone 2_b8 from P1 H49) DNA sequence | 4e-043 | |
| 2238 | Z59382 | H. sapiens CpG island DNA genomic Mse1 fragment, clone | 1e-043 | |
| | | 152b10, reverse read cpg152b10.rt1a | | |
| 2239 | L124694 | Gallus gallus (max) gene, complete cds. | 1e-043 | |
| 2240 | U72789 | Homo sapiens cosmid clone U197H5 from Xp22.1-22.2, | 5e-044 | |
| | | complete sequence [Homo sapiens] | | |
| 2241 | AF004161 | Oryctolagus cuniculus peroxisomal Ca-dependent solute | 5e-044 | |
| | | carriet mRNA, complete cds | | |
| 2242 | M22485 | Human ring chromosome 21 [r(21)] breakpoint DNA. | 2e-044 | |
| 2243 | Z60212 | H. sapiens CpG island DNA genomic Mse1 fragment, clone | 2e-045 | |
| | | 195c8, forward read cpg195c8.ft1a | | |
| 2244 | M22485 | Human ring chromosome 21 [r(21)] breakpoint DNA. | 2e-045 | |
| 2245 | AF004161 | Oryctolagus cuniculus peroxisomal Ca-dependent solute | 2e-045 | |
| | | carrier mRNA, complete cds | | |
| 2246 | D38112 | Human mitochondrial DNA,complete sequence | 1e-045 | |
| 2247 | U72787 | Homo sapiens cosmid clone U163C11 from Xp22.1-22.2, | 6e-046 | |
| | | complete sequence [Homo sapiens ] | | |
| 2248 | Z69925 | Human DNA sequence from cosmid cN116A5, on | 5e-046 | |
| | | chromosome 22q12-qter contains exon trap | | |
| 2249 | U12404 | Human Csa-19 mRNA, complete cds. | 5e-046 | |
| 2250 | Y07969 | H. sapiens mRNA for APRIL protein | 2e-046 | |
| 2251 | X90583 | H. sapiens mRNA for rat translocon-associated protein delta | 2e-046 | |
| 2252 | L35657 | Homo sapiens (subclone H8 5_a10 from P1 35 H5 C8) DNA | 2e-046 | |
| 2253 | NM_001762.1 | Homo sapiens chaperonin containing T-complex subunit 6 | 2e-046 | |
| | | (CCT6) mRNA > :: gb\|L27706\|HUMTCP20 Human | | |
| | | chaperonin protein (Tcp20) gene complete cds. | | |
| 2254 | AF008563 | Kluyveromyces lactis centromere-binding factor 5 | 7e-047 | |
| 2255 | NM_002137.1 | Homo sapiens heterogeneous nuclear ribonucleoprotein | 5e-047 | |
| | | A2/B1 (HNRPA2B1) mRNA > :: | | |
| | | gb\|M29065\|HUMRNPA2A Human hnRNP A2 protein | | |
| 2256 | NM_001866.1 | Homo sapiens cytochrome c oxidase subunit VIIb (COX7B), | 3e-047 | |
| | | nuclear gene encoding mitochondrial protein, mRNA > :: | | |
| | | emb\|Z14244\|HSCOX7BM H. sapiens coxVIIb mRNA for | | |
| | | cytochrome c oxidase subunit VIIb | | |
| 2257 | Z55938 | H. sapiens CpG island DNA genomic Mse1 fragment, clone | 2e-047 | |
| | | 74a1, reverse read cpg74a1.rt1a | | |
| 2258 | U31278 | Homo sapiens mitotic feedback control protein Madp2 | 2e-047 | |
| | | homolog mRNA, complete cds | | |
| 2259 | U72787 | Homo sapiens cosmid clone U163C11 from Xp22.1-22.2, | 2e-047 | |
| | | complete sequence [Homo sapiens] | | |
| 2260 | NM_003295.1 | Homo sapiens tumor protein, translationally-controlled 1 | 7e-048 | |
| | | (TPT1) mRNA > :: emb\|X16064\|HSTUMP Human mRNA | | |
| | | for translationally controlled tumor protein | | |
| 2261 | NM_003503.1 | Homo sapiens CDC7 (cell division cycle 7, S. cerevisiae, | 3e-048 | |
| | | homolog)-like 1 (CDC7L1) mRNA, and translated products > | | |
| | | :: gb\|AF015592\|AF015592 Homo sapiens Cdc7 | | |
| 2262 | Z62151 | H. sapiens CpG island DNA genomic Mse1 fragment, clone | 2e-048 | |
| | | 64c7, forward read cpg64c7.ft1a | | |
| 2263 | L11877 | Homo sapiens dUTP nucleotidohydrolase mRNA, 5′ end. 8e-049 | | |
| 2264 | L40403 | Homo sapiens (clone zap3) mRNA, 3′ end of cds | 3e-049 | |
| 2265 | M81600 | Human NAD(P)H:quinone oxireductase gene, exon 6 > | 2e-049 | |
| 2266 | U72787 | Homo sapiens clone U163C11 from Xp22.1-22.2, | 8e-050 | |
| | | complete sequence [Homo sapiens] | | |
| 2267 | M31004 | Human nuclear phosphoprotein B23 mRNA, clone hpB2. | 8e-050 | |
| 2268 | Z97207 | Mus musculus mRNA for B-IND1 protein | 8e-050 | |
| 2269 | AF007775 | Rattus norvegicus aquaporin-pancreas and liver | 3e-650 | |
| 2270 | U72787 | Homo sapiens cosmid clone U163C11 from Xp22.1-22.2 | 9e-051 | |
| | | complete sequence [Homo sapiens ] | | |
| 2271 | U72787 | Homo sapiens cosmid clone U163C11 from Xp22.1-22.2, | 9e-051 | |
| | | complete sequence [Homo sapiens] | | |
| 2272 | X06683 | Mouse mRNA for Cu-Zn superoxide dismutase (EC | 9e-051 | |
| | | 1.15.1.1) > :: gb\|M35725\|MUSCZSOD Mouse Cu-Zn | | |
| | | superoxide dismutase mRNA, cornplete cds. | | |
| 2273 | X93334 | H. sapiens mitochondrial DNA, complete genome | 3e-051 | |
| 2274 | U27197 | Drosophila melanogaster pelota (pelo) mRNA, complete cds | 3e-051 | |
| 2275 | D87953 | Human mRNA for RTP, complete cds | 1e-051 | |
| 2276 | L81869 | Homo sapiens (subclone 1_c4 from P1 H55) DNA sequence | 4e-052 | |
| 2277 | D63876 | Human mRNA for KIAA0154 gene, partial cds | 4e-052 | |
| 2278 | D14659 | Human mRNA for KIAA0103 gene, complete cds | 3e-052 | |
| 2279 | X93334 | H. sapiens mitochondrial DNA, complete genome | 4e-053 | |
| 2280 | D38112 | Human mitochondrial DNA, complete sequence | 4e-053 | |
| 2281 | U97519 | Homo sapiens podocalyxin-like protein mRNA, complete | 4e-053 | |
| 2282 | L35657 | Homo sapiens (subclone H8 5_a10 from P1 35 H5 C8) DNA | 4e-053 | |
| 2283 | D63876 | Human mRNA for KIAA0I54 gene, partial cds | 4e-053 | |
| 2284 | D38112 | Human mitochondrial DNA, complete sequence | 1e-053 | |
| 2285 | Z57342 | H. sapiens CpG island DNA genomc Mse1 fragment, clone | 1e-053 | |
| | | 172a12, forward read cpg172a12.ft1a | | |
| 2286 | Z64479 | H. sapiens CpG island DNA genomic Mse1 fragment, clone | 1e-053 | |
| | | 127c4, reverse read cpg127c4.rt1a | | |
| 2287 | M28209 | Homo sapiens GTP-binding protein (RAB1) mRNA, | 1e-053 | |
| 2288 | S57803 | Ro60 protein gene [human, mRNA Partial, 176 nt, segment | 4e-054 | |
| 2289 | U01139 | Mus musculus B6D2F1 clone 2C1IB mRNA. | 1e-054 | |
| 2290 | U72787 | Homo sapiens cosmid clone U163C11 from Xp22.1-22.2, | 1e-054 | |
| | | complete sequence [Homo sapiens] | | |
| 2291 | M16553 | Human tissue factor mRNA, complete cds, with an Alu | 5e-055 | |
| | | repeat in the 3′ untranslated region. | | |
| 2292 | D10522 | Homo sapiens mRNA for 80K-L protein, complete cds | 5e-055 | |
| 2293 | Z71621 | H. sapiens Wnt-13 mRNA | 5e-055 | |
| 2294 | M81104 | Human CD34 mRNA, complete cds. | 4e-055 | |
| 2295 | D29805 | Human mRNA for beta-1,4-galactosyltransferase, complete | 2e-055 | |
| 2296 | X04299 | Human mRNA for liver alcohol dehydrogenase (EC 1.1.1.1) | 2e-055 | |
| | | gamma 2 subunit from ADH3 locus | | |
| 2297 | U57715 | Rattus norvegicus FGF receptor activating protein FRAG1 | 2e-055 | |
| | | (FRAG1) mRNA, complete cds | | |
| 2298 | NM_003150.1 | Homo sapiens signal transducer and activator of | 2e-056 | |
| | | transcription 3 (acute-phase response factor) (STAT3) | | |
| | | mRNA > :: gb\|L29277\|HUMAPRF Homo sapiens DNA- | | |
| | | binding protein (APRF) mRNA, complete cds. > :: | | |
| 2299 | X56974 | M. musculus mRNA for external transcribed spacer | 2e-057 | |
| 2300 | U62435 | Human nicotinic acetylcholine receptor alpha6 subunit | 2e-057 | |
| | | precursor, mRNA, complete cds | | |
| 2301 | Z63454 | H. sapiens CpG island DNA genomic Mse1 fragment, clone | 6e-058 | |
| | | 84d2, reverse read cpg84d2.rt1a | | |
| 2302 | U34994 | Human DNA-dependent protein kinase catalytic subunit | 6e-058 | |
| 2303 | Z60432 | H. sapiens CpG island DNA genonuc Mse1 fragment, clone | 2e-058 | |
| | | 22h4, reverse read cpg22h4.rt1a | | |
| 2304 | U83590 | Rattus norvegicus PAR interacting protein mRNA, complete | 2e-058 | |
| 2305 | U83590 | Rattus norvegicus PAR interacting protein mRNA, complete | 2e-058 | |
| 2306 | M28449 | Mouse Hox-1,7 protein mRNA, 3′ end. | 8e-059 | |
| 2307 | M18981 | Human prolactin receptor-associated protein | 3e-059 | |
| 2308 | NM_001866.1 | Homo sapiens cytochrome c oxidase subunit VIIb (COX7B), | 2e-059 | |
| | | nuclear gene encoding mitochondrial protein, mRNA > :: | | |
| | | emb\|Z14244\|HSCOX7BM H. sapiens coxVIIb mRNA for | | |
| | | cytochrome c oxidase subunit VIIb | | |
| 2309 | NM_003295.1 | Homo sapiens tumor protein, translationally-controlled 1 | 2e-059 | |
| | | (TPT1) mRNA > ::emb\|X16064\|HSTUMP Human mRNA | | |
| | | for translationally controlled tumor protein | | |
| 2310 | D26067 | Human mRNA for KIAA0033 gene, partial cds | 9e-060 | |
| 2311 | NM_003130.1 | Homo sapiens sorcin (SRI) mRNA > :: | 9e-060 | |
| | | gb\|M32886\|HUMSRICPA Human sorcin CP-22 mRNA, | | |
| 2312 | X93334 | H. sapiens mitochondrial DNA, complete genome | 3e-060 | |
| 2313 | U12404 | Human Csa-19 mRNA, complete cds. | 2e-060 | |
| 2314 | AF070661 | Homo sapiens HSPC005 mRNA, complete cds | 1e-060 | |
| 2315 | U77665 | Human RNaseP protein p30 (RPP30) mRNA, complete cds | 1e-060 | |
| 2316 | L03558 | Homo sapiens cystatin B mRNA, complete cds. | 9e-061 | |
| 2317 | D14048 | Rat mRNA for SP120, complete cds | 3e-061 | |
| 2318 | NM_002450.1 | Homo sapiens metallothionein 1L (MT1L) mRNA > | 3e-061 | |
| 2319 | NM_001866.1 | Homo sapiens cytochrome c oxidase subunit VIIb (COX7B) | 4e-062 | |
| | | nuclear gene encoding mitochondrial protein, mRNA > :: | | |
| | | emb\|Z14244\|HSCOX7BM H. sapiens coxVIIb mRNA for | | |
| | | cytochrome c oxidase subunit VIIb | | |
| 2320 | AC001443 | Homo sapiens (subclone 2_f10 from BAC 2913 | 3e-062 | |
| 2321 | NM_002156.1 | Homo sapiens heat shock 6OkD protein 1 chaperonin | 3e-062 | |
| | | (HSP60) mRNA, complete cds. | | |
| 2322 | Z97207 | Mus musculus mRNA for B-IND1 protein | 3e-062 | |
| 2323 | J01415 | Human mitochondrion, complete genome | 1e-062 | |
| 2324 | D83735 | Homo sapiens mRNA for neutral calponin, complete cds | 4e-063 | |
| 2325 | NM_001866.1 | Homo sapiens cytochrome c oxidase subunit VIIb (COX7B), | 4e-063 | |
| | | nuclear gene encoding mitochondrial protein, mRNA > :: | | |
| | | emb\|Z14244\|HSCOX7BM H. sapiens coxVIIb mRNA for | | |
| | | cytochrome c oxidase subunit VIIb | | |
| 2326 | X84694 | H. sapiens mRNA for elongations factor Tu-mitochondrial | 4e-063 | |
| 2327 | NM_001910.1 | Homo sapiens cathepsin E (CTSE) mRNA > :: | 4e-063 | |
| | | gb\|J05036\|HUMCTSE Human cathepsin E mRNA, complete | | |
| 2328 | AF007775 | Rattus norvegicus aquaporin-pancreas and liver | 3e-063 | |
| 2329 | AF007775 | Rattus norvegicus aquaporin-pancreas and liver | 3e-063 | |
| 2330 | AF007862 | Mus musculus mm-Mago mRNA, complete cds | 3e-063 | |
| 2331 | D38112 | Human mitochondrial DNA, complete sequence | 1e-063 | |
| 2332 | NM_003002.1 | Homo sapiens succinate dehydrogenase complex, subunit D, | 1e-063 | |
| | | integral membrane protein (SDHD) mRNA > :: | | |
| | | dbj\|AB006202\|AB006202 Homo sapiens mRNA for | | |
| | | cytochrome b small subunit of complex H, complete cds | | |
| 2333 | D38112 | Human mitochondrial DNA, complete sequence | 1e-063 | |
| 2334 | M12759 | Human Ig J chain gene, exons 3 and 4. | 1e-063 | |
| 2335 | U07802 | Human Tis11d gene, complete cds. | 1e-063 | |
| 2336 | D14659 | Human mRNA for KIAA0103 gene, complete cds | 1e-063 | |
| 2337 | U38253 | Rattus norvegicus initiation factor eIF-B gamma subunit | 6e-064 | |
| | | (eIF-2B gamma) mRNA, complete cds | | |
| 2338 | M22146 | Human scar protein mRNA, complete cds. | 5e-064 | |
| 2339 | L20681 | Rat proto-oncogene (Ets-1) mRNA, complete cds. | 4e-064 | |
| 2340 | M14292 | Human L1Heg repetitive element ftom the intergenic region | 4e-064 | |
| | | of the epsilon and G-gamma globin genes. | | |
| 2341 | NM_003257.1 | Homo sapiens tight junction protein 1 (zona occludens 1) | 4e-064 | |
| | | (TJP1) mRNA > :: gb\|L14837\|HUMZO1A Human tight | | |
| | | junction (zonula occludens) protein ZO-1 mRNA, complete | | |
| 2342 | NM_003002.1 | Homo sapiens succinate dehydrogenase complex, subunit D | 2e-064 | |
| | | integral membrane protein (SDHD) mRNA > :: | | |
| | | dbj\|AB006202\|AB006202 Homo sapiens mRNA for | | |
| | | cytochrome b small subunit of complex II, complete cds | | |
| 2343 | X12883 | Human mRNA for cytokeratin 18 | 2e-064 | |
| 2344 | D42044 | Human for KIAA0090 gene, partial cds | 1e-064 | |
| 2345 | Z97207 | Mus musculus mRNA for B-IND1 protein | 1e-064 | |
| 2346 | X51867 | H. sapiens MRP RNA gene encoding the RNA component of | 7e-065 | |
| | | RNase MRP | | |
| 2347 | X52104 | Human mRNA for p68 protein | 7e-065 | |
| 2348 | X74215 | H. sapiens mRNA for Lon protease-like protein | 6e-065 | |
| 2349 | U20796 | Rattus norvegicus nuclear receptor Rev-ErbA-beta mRNA, | 5e-065 | |
| 2350 | X79201 | H. sapiens mRNA for SYT | 5e-065 | |
| 2351 | U34584 | Human Bcl-2 interacting killer (BIK) mRNA, complete cds | 5e-065 | |
| 2352 | Z75894 | Human DNA sequence from cosmid U61F10, between | 4e-065 | |
| | | markers DXS366 and DXS87 on chromosome X contains | | |
| 2353 | NM_002131.1 | Homo sapiens high-mobility group (nonhistone | 2e-065 | |
| | | chromosomal) protein isoforms I and Y (HMGIY) mRNA > | | |
| | | :: emb\|X14958\|HSHMGY Human hmgI mRNA for high | | |
| 2354 | AF007862 | Mus musculus mm-Mago mRNA, complete cds | 5e-068 | |
| 2355 | U74297 | Oryctolagus cuniculus PiUS mRNA, complete cds | 3e-069 | |
| 2356 | D11336 | Sus scrofa mRNA for soluble angiotesin-binding protein, | 2e-069 | |
| | | complete cds | | |
| 2357 | AF007862 | Mus musculus mm-Mago mRNA, complete cds | 2e-069 | |
| 2358 | U74297 | Oryctolagus cuniculus PiUS mRNA, complete cds | 3e-071 | |
| 2359 | AF061260 | Mus musculus immunosuperfamily protein B12 mRNA, | 5e-073 | |
| 2360 | U92949 | Mus musculus kinesin motor protein KIFC2 mRNA, | 2e-077 | |
| 2361 | AF007775 | Rattus norvegicus aquaporin-pancreas and liver | 6e-079 | |
| 2362 | AF007775 | Rattus norvegicus aquapotin-pancreas and liver | 8e-082 | |
| 2363 | U63840 | Rattus norvegicus nucleoporin p54 mRNA,complete cds | 6e-085 | |
| 2364 | U57344 | Mus musculus homeobox protein Meis3 mRNA,complete cds | 1e-085 | |
| 2365 | Z11886 | M. musculus notch-1 mRNA | 5e-087 | |
| 2366 | AF007862 | Mus musculus mm-Mago mRNA, complete cds | 2e-089 | |
| 2367 | AF007862 | Mus musculus mm-Mago mRNA, complete cds | 1e-092 | |
| 2368 | Y11092 | M. musculus mRNA for map kinase interacting kinase, Mnk2 | 2e-097 | |
| 2369 | L77991 | Gallus gallus cyclin-dependent kinase (cdk6) gene, complete | 6e-098 | |
| 2370 | U42386 | Mus musculus fibroblast growth factor inducible gene 14 | e-163 | |
| | | (FIN14) mRNA, complete cds | | |
| 2371 | U42386 | Mus musculus fibroblast growth factor inducible gene 14 | e-160 | |
| | | (FIN14) mRNA, complete cds | | |
| 2372 | U42386 | Mus musculus fibroblast growth factor inducible gene 14 | e-144 | |
| | | (FIN14) mRNA, complete cds | | |
| 2373 | AJ000696 | Rattus norvegicus mRNA for a novel kinesin-related protein, | e-106 | |
| 2374 | AJ000696 | Rattus norvegicus mRNA for a novel kinesin-related protein, | e-101 | |
| 2375 | AJ000696 | Rattus norvegicus mRNA for a novel kinesin-related protein, | e-101 | |
| 2376 | Z97207 | Mus musculus for B-IND1 protein | e-102 | |
| 2377 | AJ000696 | Rattus norvegicus mRNA for a novel kinesin-related protein, | e-122 | |
| 2378 | AB000172 | Porcine mRNA for endopeptidase 24.16, complete cds | e-118 | |
| 2379 | AB000171 | Porcine mRNA for endopeptidase 24.16, complete cds | e-131 | |
| 2380 | U38253 | Rattus norvegicus initiation factor eIF-2B gamma subunit | e-129 | |
| | | (eIF-2B gamma) mRNA, complete cds | | |
| 2381 | X54352 | M. domesticus MD6 mRNA | e-142 | |
| 2382 | X14678 | Mouse TPA-induced TIS11 mRNA | e-121 | |
| 2383 | X82632 | M. fascicularis mRNA for NAD+-isocitrate dehydrogenase | e-142 | |
| 2384 | U42385 | Mus musculus fibroblast growth factor inducible gene 16 | e-123 | |
| | | (FIN16) mRNA, complete cds | | |
| 2385 | U17901 | Rattus norvegicus phospholipase A-2-activating protein | e-116 | |
| | | (plap) mRNA, complete cds. | | |
| 2386 | U08215 | Mus musculus Hsp70-related NST-1 (hsr.1) mRNA, | e-119 | |
| 2387 | X82632 | M. fascicularis mRNA for NAD+-isocitrate dehydrogenase | e-148 | |
| 2388 | U63840 | Rattus norvegicus nucleoporin p54 mRNA, complete cds | e-131 | |
| 2389 | U42385 | Mus musculus fibroblast growth factor inducible gene 16 | e-145 | |
| | | (FIN16) mRNA, complete cds | | |
| 2390 | U42385 | Mus musculus fibroblast growth factor inducible gene 16 | e-149 | |
| | | (FIN16) mRNA, complete cds | | |
| 2391 | U81045 | Cricetulus griseus aldo-keto reductase mRNA, complete cds | e-114 | |
| 2392 | L77991 | Gallus gallus cyclin-dependent kinase (cdk6)gene, complete | e-103 | |
| 2393 | U81045 | Cricetulus griseus aldo-keto reductase mRNA, complete cds | e-115 | |
| 2394 | D30666 | Rat mRNA for brain acyl-CoA synthetase II, complete cds | e-139 | |
| 2395 | U17901 | Rattus norvegicus phospholipase A-2 activating protein | e-135 | |
| | | (plap) mRNA, complete cds. | | |
| 2396 | D30666 | Rat mRNA for brain acyl-CoA synthetase II, complete cds | e-126 | |

TABLE 5

| SEQ | CLST | Library Pair A, B | A | B | A/B | B/A |
|---|---|---|---|---|---|---|
| 1 | 734646 | 16, 17 (Colon Tumor Tissue vs. Colon Metastasis) (Colon Tumor Tissue | 14 | 0 | 14.22 | |
| | | vs. Colon Metastasis) | | | | |
| | | 15, 16 (Normal Colon vs. Colon Tumor Tissue) (Normal Colon vs. | 0 | 14 | | 13.25 |
| | | Colon Tumor Tissue) | | | | |
| 2 | 400221 | 15, 16 (Normal Colon vs. Colon Tumor Tissue) | 13 | 2 | 6.87 | |
| 3 | 205329 | 15, 16 (Normal Colon vs. Colon Tumor Tissue) | 178 | 7 | 26.88 | |
| | | 15, 17 (Normal Colon Tissue vs. Colon Metastasis) (Normal Colon | 178 | 1 | 191.06 | |
| | | Tissue vs. Colon Metastasis) | | | | |
| | | 18, 19 (Normal Colon Tissue vs. Colon Tumor) (Normal Colon Tissue | 21 | 0 | 24 | |
| | | vs. Colon Tumor) | | | | |
| | | 18, 20 (Normal Colon Tissue vs. Colon Metastasis) (Normal Colon | 21 | 0 | 17.95 | |
| | | Tissue vs. Colon Metastasis) | | | | |
| 4 | 446680 | 15, 17 (Normal Colon Tissue vs. Colon Metastasis) | 29 | 84 | | 2.7 |
| | | 23, 24 (Normal Lung Tissue vs. Lung Tumor Tissue) (Normal Lung | 40 | 94 | | 2.33 |
| | | Tissue vs. Lung Tumor Tissue) | | | | |
| 5 | 1261 | 15, 17 (Normal Colon Tissue vs. Colon Metastasis) | 0 | 7 | | 6.52 |
| | | 16, 17 (Colon Tumor Tissue vs. Colon Metastasis) | 0 | 7 | | 6.89 |
| 6 | 400258 | 16, 17 (Colon Tumor Tissue vs. Colon Metastasis) | 6 | 0 | 6.09 | |
| 7 | 450559 | 16, 17 (Colon Tumor Tissue vs. Colon Metastasis) | 2 | 11 | | 5.42 |
| 8 | 450959 | 15, 16 (Normal Colon vs. Colon Tumor Tissue) | 1 | 9 | | 8.51 |
| 9 | 451794 | 15, 16 (Normal Colon vs. Colon Tumor Tissue) | 15 | 0 | 15.85 | |
| | | 15, 17 (Normal Colon Tissue vs. Colon Metastasis) | 15 | 1 | 16.1 | |
| 10 | 415058 | 16, 17 (Colon Tumor Tissue vs. Colon Metastasis) | 0 | 6 | | 5.91 |
| 11 | 31506 | 15, 16 (Normal Colon vs. Colon Tumor Tissue) | 20 | 77 | | 3.64 |
| | | Low Met) | 5 | 0 | 6.99 | |
| | | 15, 17 (Normal Colon Tissue vs. Colon Metastasis) | 20 | 58 | | 2.7 |
| 12 | 417155 | 15, 16 (Normal Colon vs. Colon Tumor Tissue) | 6 | 0 | 6.34 | |
| 13 | 448925 | 16, 17 (Colon Tumor Tissue vs. Colon Metastasis) | 5 | 15 | | 2.95 |
| 14 | 11329 | 19, 20 (Colon Tumor Tissue vs. Colon Metastasis) (Colon Tumor Tissue | 30 | 5 | 4.49 | |
| | | vs. Colon Metastasis) | | | | |
| | | 15, 16 (Normal Colon vs. Colon Tumor Tissue) | 112 | 38 | 3.12 | |
| 15 | 650422 | 15, 17 (Normal Colon Tissue vs. Colon Metastasis) | 18 | 0 | 19.32 | |
| | | 16, 17 (Colon Tumor Tissue vs. Colon Metastasis) | 6 | 0 | 6.09 | |
| | | 15, 16 (Normal Colon vs. Colon Tumor Tissue) | 18 | 6 | 3.17 | |
| 16 | 6863 | 01, 02 (Colon, High Met vs. Colon, Low Met) | 1 | 8 | | 8.67 |
| 17 | 449690 | 16, 17 (Colon Tumor Tissue vs. Colon Metastasis) | 3 | 17 | | 5.58 |
| 18 | 724616 | 15, 16 (Normal Colon vs. Colon Tumor Tissue) | 0 | 8 | | 7.57 |
| | | 16, 17 (Colon Tumor Tissue vs. Colon Metastasis) | 8 | 0 | 8.12 | |
| 19 | 549722 | 16, 17 (Colon Tumor Tissue vs. Colon Metastasis) | 0 | 6 | | 5.91 |
| 20 | 549722 | 16, 17 (Colon Tumor Tissue vs. Colon Metastasis) | 0 | 6 | | 5.91 |
| 21 | 448110 | 15, 17 (Normal Colon Tissue vs. Colon Metastasis) | 2 | 25 | | 11.65 |
| | | 16, 17 (Colon Tumor Tissue vs. Colon Metastasis) | 1 | 25 | | 24.62 |
| 22 | 515631 | 15, 16 (Normal Colon vs. Colon Tumor Tissue) | 0 | 6 | | 5.68 |
| 23 | 11881 | 03, 04 (Breast, High Met vs. Breast, Non-Met) | 6 | 0 | 5.85 | |
| 24 | 650856 | 16, 17 (Colon Tumor Tissue vs. Colon Metastasis) | 6 | 0 | 6.09 | |
| 25 | 449701 | 16, 17 (Colon Tumor Tissue vs. Colon Metastasis) | 17 | 1 | 17.26 | |
| | | 15, 16 (Normal Colon vs. Colon Tumor Tissue) | 1 | 17 | | 16.08 |
| 26 | 651073 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 27 | 10340 | 03, 04 (Breast, High Met vs. Breast, Non-Met) | 6 | 0 | 5.85 | |
| 28 | 648310 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 29 | 730336 | 15, 16 (Normal Colon vs. Colon Tumor Tissue) | 0 | 24 | | 22.71 |
| | | 16, 17 (Colon Tumor Tissue vs. Colon Metastasis) | 24 | 0 | 24.37 | |
| 30 | 3060 | 16, 17 (Colon Tumor Tissue vs. Colon Metastasis) | 4 | 17 | | 4.19 |
| | | 15, 16 (Normal Colon vs. Colon Tumor Tissue) | 14 | 4 | 3.7 | |
| | | 03, 04 (Breast, High Met vs. Breast, Non-Met) | 0 | 11 | | 11.27 |
| 31 | 453016 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 33 | 185461 | 03, 04 (Breast, High Met vs. Breast, Non-Met) | 0 | 17 | | 17.42 |
| 34 | 452530 | 15, 16 (Normal Colon vs. Colon Tumor Tissue) | 10 | 2 | 5.28 | |
| 35 | 448925 | 16, 17 (Colon Tumor Tissue vs. Colon Metastasis) | 5 | 15 | | 2.95 |
| 36 | 1013 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| | | 01, 02 (Colon, High Met vs. Colon, Low Met) | 40 | 84 | | 2.28 |
| 37 | 6545 | 03, 04 (Breast, High Met vs. Breast, Non-Met) | 0 | 9 | | 9.22 |
| 38 | 449891 | 15, 16 (Normal Colon vs. Colon Tumor Tissue) | 8 | 1 | 8.46 | |
| 39 | 4045 | 01, 02 (Colon, High Met vs. Colon, Low Met) | 2 | 11 | | 5.96 |
| | | 03, 04 (Breast, High Met vs. Breast, Non-Met) | 10 | 1 | 9.76 | |
| 40 | 404475 | 16, 17 (Colon Tumor Tissue vs. Colon Metastasis) | 11 | 2 | 5.59 | |
| | | 15, 17 (Normal Colon Tissue vs. Colon Metastasis) | 19 | 2 | 10.2 | |
| 41 | 650297 | 15, 17 (Normal Colon Tissue vs. Colon Metastasis) | 6 | 0 | 6.44 | |
| 42 | 650493 | 15, 17 (Normal Colon Tissue vs. Colon Metastasis) | 7 | 0 | 7.51 | |
| 43 | 644884 | 15, 17 (Normal Colon Tissue vs. Colon Metastasis) | 11 | 0 | 11.81 | |
| | | 16, 17 (Colon Tumor Tissue vs. Colon Metastasis) | 6 | 0 | 6.09 | |
| 44 | 452212 | 15, 16 (Normal Colon vs. Colon Tumor Tissue) | 6 | 0 | 6.34 | |
| 45 | 402727 | 15, 17 (Normal Colon Tissue vs. Colon Metastasis) | 5 | 17 | | 3.17 |
| 46 | 645194 | 15, 17 (Normal Colon Tissue vs. Colon Metastasis) | 7 | 0 | 7.51 | |
| 47 | 447501 | 15, 17 (Normal Colon Tissue vs. Colon Metastasis) | 15 | 3 | 5.37 | |
| 48 | 556326 | 16, 17 (Colon Tumor Tissue vs. Colon Metastasis) | 0 | 8 | | 7.88 |
| 49 | 447035 | 16, 17 (Colon Tumor Tissue vs. Colon Metastasis) | 8 | 1 | 8.12 | |
| 50 | 2551 | 16, 17 (Colon Tumor Tissue vs. Colon Metastasis) | 6 | 0 | 6.09 | |
| 51 | 736154 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 52 | 452028 | 15, 17 (Normal Colon Tissue vs. Colon Metastasis) | 0 | 7 | | 6.52 |
| 53 | 447441 | 15, 17 (Normal Colon Tissue vs. Colon Metastasis) | 34 | 129 | | 3.53 |
| | | 16, 17 (Colon Tumor Tissue vs. Colon Metastasis) | 34 | 129 | | 3.74 |
| | | 19, 20 (Colon Tumor Tissue vs. Colon Metastasis) | 1 | 8 | | 10.7 |
| | | 23, 24 (Normal Lung Tissue vs. Lung Tumor Tissue) | 155 | 32 | 4.89 | |
| 54 | 11028 | 01, 02 (Colon, High Met vs. Colon, Low Met) (Colon, High Met vs. | 0 | 6 | | 6.5 |
| | | Colon, Low Met) | | | | |
| 55 | 640974 | 15, 17 (Normal Colon Tissue vs. Colon Metastasis) | 9 | 0 | 9.66 | |
| 56 | 555103 | 15, 17 (Normal Colon Tissue vs. Colon Metastasis) | 0 | 7 | | 6.52 |
| | | 23, 24 (Normal Lung Tissue vs. Lung Tumor Tissue) | 0 | 6 | | 5.94 |
| 57 | 446789 | 15, 16 (Normal Colon vs. Colon Tumor Tissue) | 16 | 5 | 3.38 | |
| 58 | 644884 | 15, 17 (Normal Colon Tissue vs. Colon Metastasis) | 11 | 0 | 11.81 | |
| | | 16, 17 (Colon Tumor Tissue vs. Colon Metastasis) | 6 | 0 | 6.09 | |
| 59 | 9029 | 01, 02 (Colon, High Met vs. Colon, Low Met) | 7 | 0 | 6.46 | |
| 60 | 419255 | 15, 16 (Normal Colon vs. Colon Tumor Tissue) | 11 | 0 | 11.63 | |
| | | 15, 17 (Normal Colon Tissue vs. Colon Metastasis) | 11 | 1 | 11.81 | |
| 61 | 4309 | 01, 02 (Colon, High Met vs. Colon, Low Met) | 4 | 13 | | 3.52 |
| 62 | 554069 | 16, 17 (Colon Tumor Tissue vs. Colon Metastasis) | 0 | 6 | | 5.91 |
| 63 | 4330 | 03, 04 (Breast, High Met vs. Breast, Non-Met) (Breast, High Met vs. | 13 | 3 | 4.23 | |
| | | Breast, Non-Met) | | | | |
| | | 01, 02 (Colon, High Met vs. Colon, Low Met) | 1 | 10 | | 10.84 |
| 64 | 644903 | 15, 17 (Normal Colon Tissue vs. Colon Metastasis) | 7 | 0 | 7.51 | |
| 65 | 549395 | 15, 17 (Normal Colon Tissue vs. Colon Metastasis) | 2 | 13 | | 6.06 |
| | | 16, 17 (Colon Tumor Tissue vs. Colon Metastasis) | 1 | 13 | | 12.8 |
| 66 | 4974 | 01, 02 (Colon, High Met vs. Colon, Low Met) | 1 | 8 | | 8.67 |
| 67 | 447466 | 15, 16 (Normal Colon vs. Colon Tumor Tissue) | 155 | 5 | 32.77 | |
| | | 18, 19 (Normal Colon Tissue vs. Colon Tumor) | 16 | 1 | 18.28 | |
| | | 15, 17 (Normal Colon Tissue vs. Colon Metastasis) | 155 | 2 | 83.19 | |
| | | 18, 20 (Normal Colon Tissue vs. Colon Metastasis) | 16 | 0 | 13.68 | |
| 68 | 645073 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 69 | 447978 | 15, 17 (Normal Colon Tissue vs. Colon Metastasis) | 0 | 8 | | 7.45 |
| 70 | 607430 | 15, 16 (Normal Colon vs. Colon Tumor Tissue) | 6 | 0 | 6.34 | |
| 71 | 556198 | 15, 16 (Normal Colon vs. Colon Tumor Tissue) | 0 | 6 | | 5.68 |
| 72 | 450323 | 15, 17 (Normal Colon Tissue vs. Colon Metastasis) | 0 | 8 | | 7.45 |
| 73 | 21205 | 03, 04 (Breast, High Met vs. Breast, Non-Met) | 1 | 9 | | 9.22 |
| 74 | 561109 | 15, 16 (Normal Colon vs. Colon Tumor Tissue) | 0 | 8 | | 7.57 |
| 75 | 446673 | 16, 17 (Colon Tumor Tissue vs. Colon Metastasis) | 8 | 1 | 8.12 | |
| 76 | 456026 | 15, 16 (Normal Colon vs. Colon Tumor Tissue) | 24 | 4 | 6.34 | |
| | | 15, 17 (Normal Colon Tissue vs. Colon Metastasis) | 24 | 8 | 3.22 | |
| 77 | 449142 | 16, 17 (Colon Tumor Tissue vs. Colon Metastasis) | 9 | 1 | 9.14 | |
| 78 | 5830 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 79 | 554109 | 15, 16 (Normal Colon vs. Colon Tumor Tissue) | 15 | 2 | 7.93 | |
| | | 16, 17 (Colon Tumor Tissue vs. Colon Metastasis) | 2 | 11 | | 5.42 |
| 80 | 595506 | 15, 16 (Normal Colon vs. Colon Tumor Tissue) | 0 | 6 | | 5.68 |
| 81 | 453981 | 23, 24 (Normal Lung Tissue vs. Lung Tumor Tissue) | 43 | 258 | | 5.94 |
| | | 19, 20 (Colon Tumor Tissue vs. Colon Metastasis) | 2 | 110 | | 73.53 |
| | | 16, 17 (Colon Tumor Tissue vs. Colon Metastasis) | 590 | 6 | 99.86 | |
| | | 15, 17 (Normal Colon Tissue vs. Colon Metastasis) | 68 | 6 | 12.16 | |
| | | 15, 16 (Normal Colon vs. Colon Tumor Tissue) | 68 | 590 | | 8.21 |
| | | 18, 20 (Normal Colon Tissue vs. Colon Metastasis) | 0 | 110 | | 128.69 |
| 82 | 642461 | 15, 17 (Normal Colon Tissue vs. Colon Metastasis) | 14 | 0 | 15.03 | |
| | | 16, 17 (Colon Tumor Tissue vs. Colon Metastasis) | 10 | 0 | 10.16 | |
| 83 | 556198 | 15, 16 (Normal Colon vs. Colon Tumor Tissue) | 0 | 6 | | 5.68 |
| 84 | 2082 | 15, 16 (Normal Colon vs. Colon Tumor Tissue) | 0 | 7 | | 6.62 |
| | | 03, 04 (Breast, High Met vs. Breast, Non-Met) | 15 | 55 | | 3.76 |
| | | 08, 09 (Lung, High Met vs. Lung, Low Met) | 7 | 38 | | 3.88 |
| 85 | 549435 | 15, 16 (Normal Colon vs. Colon Tumor Tissue) | 14 | 2 | 7.4 | |
| 86 | 2286 | 15, 16 (Normal Colon vs. Colon Tumor Tissue) | 5 | 28 | | 5.3 |
| | | 19, 20 (Colon Tumor Tissue vs. Colon Metastasis) | 13 | 2 | 4.86 | |
| | | 15, 17 (Normal Colon Tissue vs. Colon Metastasis) | 5 | 19 | | 3.54 |
| | | 18, 19 (Normal Colon Tissue vs. Colon Tumor) | 3 | 13 | | 3.79 |
| 87 | 2737 | 16, 17 (Colon Tumor Tissue vs. Colon Metastasis) | 3 | 14 | | 4.6 |
| | | 15, 17 (Normal Colon Tissue vs. Colon Metastasis) | 4 | 14 | | 3.26 |
| 88 | 728115 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 89 | 650856 | 16, 17 (Colon Tumor Tissue vs. Colon Metastasis) | 6 | 0 | 6.09 | |
| 90 | 650476 | 15, 17 (Normal Colon Tissue vs. Colon Metastasis) | 7 | 0 | 7.51 | |
| 91 | 535208 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 92 | 733849 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 93 | 447978 | 15, 17 (Normal Colon Tissue vs. Colon Metastasis) | 0 | 8 | | 7.45 |
| 94 | 729483 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 95 | 12018 | 03, 04 (Breast, High Met vs. Breast, Non-Met) | 0 | 6 | | 6.15 |
| 96 | 4747 | 01, 02 (Colon, High Met vs. Colon, Low Met) | 2 | 10 | | 5.42 |
| 97 | 4747 | 01, 02 (Colon, High Met vs. Colon, Low Met) | 2 | 10 | | 5.42 |
| 98 | 185577 | 03, 04 (Breast, High Met vs. Breast, Non-Met) | 0 | 8 | | 8.2 |
| 99 | 4126 | 03, 04 (Breast, High Met vs. Breast, Non-Met) | 13 | 1 | 12.68 | |
| 100 | 11456 | 01, 02 (Colon, High Met vs. Colon, Low Met) | 0 | 6 | | 6.5 |
| 101 | 729851 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 102 | 449849 | 16, 17 (Colon Tumor Tissue vs. Colon Metastasis) | 0 | 6 | | 5.91 |
| 103 | 2490 | 08, 09 (Lung, High Met vs. Lung, Low Met) | 7 | 1 | 9.78 | |
| | | 01, 02 (Colon, High Met vs. Colon, Low Met) | 21 | 6 | 3.23 | |
| | | 03, 04 (Breast, High Met vs. Breast, Non-Met) | 14 | 3 | 4.55 | |
| 104 | 549041 | 16, 17 (Colon Tumor Tissue vs. Colon Metastasis) | 0 | 6 | | 5.91 |
| 105 | 11881 | 03, 04 (Breast, High Met vs. Breast, Non-Met) | 6 | 0 | 5.85 | |
| 106 | 724296 | 15, 16 (Normal Colon vs. Colon Tumor Tissue) | 0 | 128 | | 121.1 |
| | | 16, 17 (Colon Tumor Tissue vs. Colon Metastasis) | 128 | 0 | 129.99 | |
| 107 | 726173 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 108 | 2423 | 03, 04 (Breast, High Met vs. Breast, Non-Met) | 14 | 0 | 13.66 | |
| 109 | 556250 | 15, 16 (Normal Colon vs. Colon Tumor Tissue) | 1 | 28 | | 26.49 |
| | | 16, 17 (Colon Tumor Tissue vs. Colon Metastasis) | 28 | 4 | 7.11 | |
| 110 | 643594 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 111 | 11881 | 03, 04 (Breast, High Met vs. Breast, Non-Met) | 6 | 0 | 5.85 | |
| 112 | 7436 | 03, 04 (Breast, High Met vs. Breast, Non-Met) | 9 | 1 | 8.78 | |
| 113 | 2110 | 03, 04 (Breast, High Met vs. Breast, Non-Met) | 31 | 5 | 6.05 | |
| 114 | 10340 | 03, 04 (Breast, High Met vs. Breast, Non-Met) | 6 | 0 | 5.85 | |
| 115 | 643594 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 116 | 447035 | 16, 17 (Colon Tumor Tissue vs. Colon Metastasis) | 8 | 1 | 8.12 | |
| 117 | 402707 | 15, 17 (Normal Colon Tissue vs. Colon Metastasis) | 1 | 9 | | 8.38 |
| 118 | 645799 | 16, 17 (Colon Tumor Tissue vs. Colon Metastasis) | 7 | 0 | 7.11 | |
| 119 | 171511 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 120 | 451607 | 16, 17 (Colon Tumor Tissue vs. Colon Metastasis) | 1 | 9 | | 8.86 |
| 121 | 3138 | 03, 04 (Breast, High Met vs. Breast, Non-Met) | 1 | 10 | | 10.25 |
| 122 | 2988 | 01, 02 (Colon, High Met vs. Colon, Low Met) | 2 | 11 | | 5.96 |
| 123 | 447326 | 15, 17 (Normal Colon Tissue vs. Colon Metastasis) | 6 | 0 | 6.44 | |
| 124 | 561734 | 15, 16 (Normal Colon vs. Colon Tumor Tissue) | 0 | 6 | | 5.68 |
| 125 | 454999 | 15, 17 (Normal Colon Tissue vs. Colon Metastasis) | 28 | 15 | 2 | |
| | | 15, 16 (Normal Colon vs. Colon Tumor Tissue) | 28 | 11 | 2.69 | |
| 126 | 185652 | 03, 04 (Breast, High Met vs. Breast, Non-Met) | 0 | 7 | | 7.17 |
| 127 | 6725 | 03, 04 (Breast, High Met vs. Breast, Non-Met) | 10 | 0 | 9.76 | |
| 128 | 726644 | 15, 16 (Normal Colon vs. Colon Tumor Tissue) | 0 | 18 | | 17.03 |
| | | 16, 17 (Colon Tumor Tissue vs. Colon Metastasis) | 18 | 0 | 18.28 | |
| 129 | 11012 | 01, 02 (Colon, High Met vs. Colon, Low Met) | 0 | 6 | | 6.5 |
| 130 | 726377 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 131 | 735326 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 132 | 650845 | 16, 17 (Colon Tumor Tissue vs. Colon Metastasis) | 6 | 0 | 6.09 | |
| 133 | 9048 | 01, 02 (Colon, High Met vs. Colon, Low Met) | 7 | 0 | 6.46 | |
| 134 | 732254 | 15, 16 (Normal Colon vs. Colon Tumor Tissue) | 0 | 6 | | 5.68 |
| | | 23, 24 (Normal Lung Tissue vs. Lung Tumor Tissue) | 0 | 8 | | 7.92 |
| | | 16, 17 (Colon Tumor Tissue vs. Colon Metastasis) | 6 | 0 | 6.09 | |
| 135 | 452052 | 15, 17 (Normal Colon Tissue vs. Colon Metastasis) | 8 | 1 | 8.59 | |
| 136 | 554079 | 16, 17 (Colon Tumor Tissue vs. Colon Metastasis) | 2 | 10 | | 4.92 |
| 137 | 9049 | 01, 02 (Colon, High Met vs. Colon, Low Met) | 7 | 0 | 6.46 | |
| 138 | 1307 | 03, 04 (Breast, High Met vs. Breast, Non-Met) | 14 | 89 | | 6.52 |
| 139 | 139730 | 03, 04 (Breast, High Met vs. Breast, Non-Met) | 0 | 9 | | 9.22 |
| 140 | 7750 | 03, 04 (Breast, High Met vs. Breast, Non-Met) | 1 | 14 | | 14.35 |
| 141 | 8050 | 01, 02 (Colon, High Met vs. Colon, Low Met) | 0 | 7 | | 7.59 |
| 142 | 725222 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 143 | 3275 | 03, 04 (Breast, High Met vs. Breast, Non-Met) | 8 | 0 | 7.81 | |
| 144 | 7424 | 03, 04 (Breast, High Met vs. Breast, Non-Met) | 6 | 0 | 5.85 | |
| 145 | 8953 | 03, 04 (Breast, High Met vs. Breast, Non-Met) | 0 | 13 | | 13.32 |
| 146 | 8966 | 03, 04 (Breast, High Met vs. Breast, Non-Met) | 0 | 7 | | 7.17 |
| 147 | 530883 | 15, 16 (Normal Colon vs. Colon Tumor Tissue) | 0 | 7 | | 6.62 |
| 148 | 6725 | 03, 04 (Breast, High Met vs. Breast, Non-Met) | 10 | 0 | 9.76 | |
| 149 | 4439 | 08, 09 (Lung, High Met vs. Lung, Low Met) | 10 | 2 | 6.99 | |
| | | 01, 02 (Colon, High Met vs. Colon, Low Met) | 15 | 2 | 6.92 | |
| 150 | 648472 | 15, 17 (Normal Colon Tissue vs. Colon Metastasis) | 7 | 0 | 7.51 | |
| 151 | 735346 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 152 | 732121 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 153 | 650337 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 154 | 533588 | 15, 17 (Normal Colon Tissue vs. Colon Metastasis) | 7 | 0 | 7.51 | |
| 155 | 649667 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 156 | 394436 | 15, 17 (Normal Colon Tissue vs. Colon Metastasis) | 7 | 0 | 7.51 | |
| 157 | 649354 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 158 | 2022 | 03, 04 (Breast, High Met vs. Breast, Non-Met) | 5 | 15 | | 3.07 |
| | | 21, 22 (Normal Prostate vs. Prostate Cancer) | 0 | 6 | | 6.1 |
| 159 | 561359 | 15, 17 (Normal Colon Tissue vs. Colon Metastasis) | 12 | 3 | 4.29 | |
| | | 15, 16 (Normal Colon vs. Colon Tumor Tissue) | 12 | 2 | 6.34 | |
| 160 | 7607 | 15, 17 (Normal Colon Tissue vs. Colon Metastasis) | 13 | 3 | 4.65 | |
| | | 01, 02 (Colon, High Met vs. Colon, Low Met) | 0 | 7 | | 7.59 |
| | | 15, 16 (Normal Colon vs. Colon Tumor Tissue) | 13 | 4 | 3.44 | |
| 161 | 7750 | 03, 04 (Breast, High Met vs. Breast, Non-Met) | 1 | 14 | | 14.35 |
| 162 | 410554 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 163 | 2315 | 03, 04 (Breast, High Met vs. Breast, Non-Met) | 7 | 118 | | 17.28 |
| | | 01, 02 (Colon, High Met vs. Colon, Low Met) | 25 | 4 | 5.76 | |
| 164 | 561734 | 15, 16 (Normal Colon vs. Colon Tumor Tissue) | 0 | 6 | | 5.68 |
| 165 | 4420 | 03, 04 (Breast, High Met vs. Breast, Non-Met) | 6 | 0 | 5.85 | |
| | | 01, 02 (Colon, High Met vs. Colon, Low Met) | 1 | 10 | | 10.84 |
| 166 | 559663 | 15, 16 (Normal Colon vs. Colon Tumor Tissue) | 15 | 4 | 3.96 | |
| 167 | 7082 | 03, 04 (Breast, High Met vs. Breast, Non-Met) | 10 | 0 | 9.76 | |
| 168 | 2315 | 03, 04 (Breast, High Met vs. Breast, Non-Met) | 7 | 118 | | 17.28 |
| | | 01, 02 (Colon, High Met vs. Colon, Low Met) | 25 | 4 | 5.76 | |
| 169 | 650472 | 15, 17 (Normal Colon Tissue vs. Colon Metastasis) | 6 | 0 | 6.44 | |
| 170 | 6482 | 01, 02 (Colon, High Met vs. Colon, Low Met) | 0 | 6 | | 6.5 |
| 171 | 4584 | 01, 02 (Colon, High Met vs. Colon, Low Met) | 1 | 11 | | 11.93 |
| 172 | 453846 | 15, 17 (Normal Colon Tissue vs. Colon Metastasis) | 0 | 11 | | 10.25 |
| 173 | 650820 | 16, 17 (Colon Tumor Tissue vs. Colon Metastasis) | 8 | 0 | 8.12 | |
| 174 | 642906 | 16, 17 (Colon Tumor Tissue vs. Colon Metastasis) | 7 | 0 | 7.11 | |
| 175 | 448805 | 16, 17 (Colon Tumor Tissue vs. Colon Metastasis) | 6 | 20 | | 3.28 |
| 176 | 649667 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 177 | 735786 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 178 | 121457 | 08, 09 (Lung, High Met vs. Lung, Low Met) | 91 | 359 | | 2.82 |
| | | 15, 17 (Normal Colon Tissue vs. Colon Metastasis) | 2 | 11 | | 5.12 |
| 179 | 372960 | 15, 17 (Normal Colon Tissue vs. Colon Metastasis) | 6 | 18 | | 2.79 |
| | | 15, 16 (Normal Colon vs. Colon Tumor Tissue) | 6 | 33 | | 5.2 |
| 180 | 120049 | 15, 17 (Normal Colon Tissue vs. Colon Metastasis) | 10 | 2 | 5.37 | |
| 181 | 648996 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 182 | 3765 | 01, 02 (Colon, High Met vs. Colon, Low Met) | 19 | 6 | 2.92 | |
| 183 | 462642 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 184 | 727181 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 185 | 649259 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 186 | 649717 | 16, 17 (Colon Tumor Tissue vs. Colon Metastasis) | 6 | 0 | 6.09 | |
| 187 | 736860 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 188 | 729175 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 189 | 642906 | 16, 17 (Colon Tumor Tissue vs. Colon Metastasis) | 7 | 0 | 7.11 | |
| 190 | 4420 | 03, 04 (Breast, High Met vs. Breast, Non-Met) | 6 | 0 | 5.85 | |
| | | 01, 02 (Colon, High Met vs. Colon, Low Met) | 1 | 10 | | 10.84 |
| 191 | 2420 | 01, 02 (Colon, High Met vs. Colon, Low Met) | 28 | 11 | 2.35 | |
| 192 | 648109 | 15, 17 (Normal Colon Tissue vs. Colon Metastasis) | 8 | 0 | 8.59 | |
| | | 15, 16 (Normal Colon vs. Colon Tumor Tissue) | 8 | 1 | 8.46 | |
| 193 | 2334 | 15, 16 (Normal Colon vs. Colon Tumor Tissue) | 0 | 6 | | 5.68 |
| 194 | 639705 | 15, 16 (Normal Colon vs. Colon Tumor Tissue) | 10 | 0 | 10.57 | |
| | | 15, 17 (Normal Colon Tissue vs. Colon Metastasis) | 10 | 0 | 10.73 | |
| 195 | 551907 | 15, 16 (Normal Colon vs. Colon Tumor Tissue) | 7 | 0 | 7.4 | |
| 196 | 561382 | 15, 16 (Normal Colon vs. Colon Tumor Tissue) | 12 | 2 | 6.34 | |
| 197 | 595506 | 15, 16 (Normal Colon vs. Colon Tumor Tissue) | 0 | 6 | | 5.68 |
| 198 | 499424 | 15, 16 (Normal Colon vs. Colon Tumor Tissue) | 0 | 8 | | 7.57 |
| 199 | 735477 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 200 | 734370 | 15, 16 (Normal Colon vs. Colon Tumor Tissue) | 0 | 11 | | 10.41 |
| | | 16, 17 (Colon Tumor Tissue vs. Colon Metastasis) | 11 | 0 | 11.17 | |
| 201 | 779 | 01, 02 (Colon, High Met vs. Colon, Low Met) | 27 | 54 | | 2.17 |
| | | 03, 04 (Breast, High Met vs. Breast, Non-Met) | 60 | 22 | 2.66 | |
| 202 | 649143 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 203 | 489 | 15, 17 (Normal Colon Tissue vs. Colon Metastasis) | 12 | 58 | | 4.5 |
| | | 15, 16 (Normal Colon vs. Colon Tumor Tissue) | 12 | 58 | | 4.57 |
| 204 | 2994 | 01, 02 (Colon, High Met vs. Colon, Low Met) | 3 | 14 | | 5.06 |
| 205 | 2994 | 01, 02 (Colon, High Met vs. Colon, Low Met) | 3 | 14 | | 5.06 |
| 206 | 11147 | 01, 02 (Colon, High Met vs. Colon, Low Met) | 0 | 6 | | 6.5 |
| 207 | 549395 | 15, 17 (Normal Colon Tissue vs. Colon Metastasis) | 2 | 13 | | 6.06 |
| | | 16, 17 (Colon Tumor Tissue vs. Colon Metastasis) | 1 | 13 | | 12.8 |
| 208 | 559806 | 16, 17 (Colon Tumor Tissue vs. Colon Metastasis) | 0 | 6 | | 5.91 |
| 209 | 452238 | 15, 16 (Normal Colon vs. Colon Tumor Tissue) | 0 | 6 | | 5.68 |
| 210 | 225914 | 16, 17 (Colon Tumor Tissue vs. Colon Metastasis) | 0 | 6 | | 5.91 |
| 211 | 463480 | 16, 17 (Colon Tumor Tissue vs. Colon Metastasis) | 0 | 6 | | 5.91 |
| 212 | 184725 | 15, 17 (Normal Colon Tissue vs. Colon Metastasis) | 8 | 0 | 8.59 | |
| | | 16, 17 (Colon Tumor Tissue vs. Colon Metastasis) | 7 | 0 | 7.11 | |
| 213 | 557401 | 15, 17 (Normal Colon Tissue vs. Colon Metastasis) | 10 | 2 | 5.37 | |
| 214 | 455155 | 15, 16 (Normal Colon vs. Colon Tumor Tissue) | 12 | 3 | 4.23 | |
| 215 | 551117 | 16, 17 (Colon Tumor Tissue vs. Colon Metastasis) | 0 | 6 | | 5.91 |
| 217 | 729295 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 218 | 450429 | 16, 17 (Colon Tumor Tissue vs. Colon Metastasis) | 10 | 1 | 10.16 | |
| | | 15, 17 (Normal Colon Tissue vs. Colon Metastasis) | 13 | 1 | 13.95 | |
| 219 | 450148 | 16, 17 (Colon Tumor Tissue vs. Colon Metastasis) | 0 | 6 | | 5.91 |
| 220 | 380412 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 221 | 446614 | 16, 17 (Colon Tumor Tissue vs. Colon Metastasis) | 7 | 0 | 7.11 | |
| 222 | 555911 | 15, 17 (Normal Colon Tissue vs. Colon Metastasis) | 2 | 21 | | 9.78 |
| | | 16, 17 (Colon Tumor Tissue vs. Colon Metastasis) | 1 | 21 | | 20.68 |
| 223 | 450828 | 15, 17 (Normal Colon Tissue vs. Colon Metastasis) | 1 | 9 | | 8.38 |
| 224 | 28 | 18, 20 (Normal Colon Tissue vs. Colon Metastasis) | 2 | 11 | | 6.43 |
| | | 18, 19 (Normal Colon Tissue vs. Colon Tumor) | 2 | 43 | | 18.81 |
| | | 15, 17 (Normal Colon Tissue vs. Colon Metastasis) | 53 | 207 | | 3.64 |
| | | 03, 04 (Breast, High Met vs. Breast, Non-Met) | 697 | 1789 | | 2.63 |
| | | 19, 20 (Colon Tumor Tissue vs. Colon Metastasis) | 43 | 11 | 2.92 | |
| 225 | 446450 | 15, 16 (Normal Colon vs. Colon Tumor Tissue) | 11 | 3 | 3.88 | |
| 226 | 452026 | 15, 16 (Normal Colon vs. Colon Tumor Tissue) | 35 | 14 | 2.64 | |
| 227 | 643594 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 228 | 1905 | 01, 02 (Colon, High Met vs. Colon, Low Met) | 7 | 21 | | 3.25 |
| | | 15, 17 (Normal Colon Tissue vs. Colon Metastasis) | 0 | 7 | | 6.52 |
| 229 | 651073 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 230 | 553705 | 15, 16 (Normal Colon vs. Colon Tumor Tissue) | 12 | 0 | 12.68 | |
| 231 | 521840 | 15, 16 (Normal Colon vs. Colon Tumor Tissue) | 6 | 0 | 6.34 | |
| 232 | 648689 | 16, 17 (Colon Tumor Tissue vs. Colon Metastasis) | 6 | 0 | 6.09 | |
| 233 | 447858 | 15, 16 (Normal Colon vs. Colon Tumor Tissue) | 12 | 3 | 4.23 | |
| 234 | 556198 | 15, 16 (Normal Colon vs. Colon Tumor Tissue) | 0 | 6 | | 5.68 |
| 235 | 394436 | 15, 17 (Normal Colon Tissue vs. Colon Metastasis) | 7 | 0 | 7.51 | |
| 236 | 639651 | 15, 17 (Normal Colon Tissue vs. Colon Metastasis) | 6 | 0 | 6.44 | |
| 237 | 499424 | 15, 16 (Normal Colon vs. Colon Tumor Tissue) | 0 | 8 | | 7.57 |
| 238 | 468109 | 15, 17 (Normal Colon Tissue vs. Colon Metastasis) | 6 | 0 | 6.44 | |
| 239 | 185701 | 03, 04 (Breast, High Met vs. Breast, Non-Met) | 0 | 6 | | 6.15 |
| 240 | 451811 | 15, 17 (Normal Colon Tissue vs. Colon Metastasis) | 10 | 2 | 5.37 | |
| 241 | 730670 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 242 | 172013 | 15, 16 (Normal Colon vs. Colon Tumor Tissue) | 6 | 0 | 6.34 | |
| 243 | 449142 | 16, 17 (Colon Tumor Tissue vs. Colon Metastasis) | 9 | 1 | 9.14 | |
| 244 | 446964 | 15, 17 (Normal Colon Tissue vs. Colon Metastasis) | 1 | 10 | | 9.32 |
| 245 | 414739 | 16, 17 (Colon Tumor Tissue vs. Colon Metastasis) | 2 | 14 | | 6.89 |
| | | 15, 17 (Normal Colon Tissue vs. Colon Metastasis) | 2 | 14 | | 6.52 |
| 246 | 641124 | 15, 17 (Normal Colon Tissue vs. Colon Metastasis) | 6 | 0 | 6.44 | |
| 247 | 555702 | 15, 16 (Normal Colon vs. Colon Tumor Tissue) | 11 | 2 | 5.81 | |
| 248 | 549435 | 15, 16 (Normal Colon vs. Colon Tumor Tissue) | 14 | 2 | 7.4 | |
| 249 | 643954 | 16, 17 (Colon Tumor Tissue vs. Colon Metastasis) | 6 | 0 | 6.09 | |
| 250 | 5984 | 16, 17 (Colon Tumor Tissue vs. Colon Metastasis) | 0 | 6 | | 5.91 |
| 251 | 560526 | 16, 17 (Colon Tumor Tissue vs. Colon Metastasis) | 0 | 8 | | 7.88 |
| 252 | 411113 | 16, 17 (Colon Tumor Tissue vs. Colon Metastasis) | 0 | 6 | | 5.91 |
| 253 | 7607 | 15, 16 (Normal Colon vs. Colon Tumor Tissue) | 13 | 4 | 3.44 | |
| | | 01, 02 (Colon, High Met vs. Colon, Low Met) | 0 | 7 | | 7.59 |
| | | 15, 17 (Normal Colon Tissue vs. Colon Metastasis) | 13 | 3 | 4.65 | |
| 254 | 559409 | 16, 17 (Colon Tumor Tissue vs. Colon Metastasis) | 0 | 6 | | 5.91 |
| 255 | 650053 | 15, 16 (Normal Colon vs. Colon Tumor Tissue) | 10 | 1 | 10.57 | |
| | | 15, 17 (Normal Colon Tissue vs. Colon Metastasis) | 10 | 0 | 10.73 | |
| 256 | 448511 | 15, 17 (Normal Colon Tissue vs. Colon Metastasis) | 3 | 26 | | 8.07 |
| | | 16, 17 (Colon Tumor Tissue vs. Colon Metastasis) | 4 | 26 | | 6.4 |
| 257 | 642142 | 16, 17 (Colon Tumor Tissue vs. Colon Metastasis) | 11 | 0 | 11.17 | |
| | | l5, 16 (Normal Colon vs. Colon Tumor Tissue) | 2 | 11 | | 5.2 |
| 258 | 470462 | 15, 17 (Normal Colon Tissue vs. Colon Metastasis) | 7 | 0 | 7.51 | |
| 259 | 431601 | 18, 20 (Normal Colon Tissue vs. Colon Metastasis) | 9 | 0 | 7.69 | |
| | | 15, 17 (Normal Colon Tissue vs. Colon Metastasis) | 28 | 9 | 3.34 | |
| | | 15, 16 (Normal Colon vs. Colon Tumor Tissue) | 28 | 6 | 4.93 | |
| | | 08, 09 (Lung, High Met vs. Lung, Low Met) | 5 | 0 | 6.99 | |
| 260 | 421431 | 15, 16 (Normal Colon vs. Colon Tumor Tissue) | 8 | 1 | 8.46 | |
| 261 | 284586 | 15, 17 (Normal Colon Tissue vs. Colon Metastasis) | 6 | 0 | 6.44 | |
| 262 | 556198 | 15, 16 (Normal Colon vs. Colon Tumor Tissue) | 0 | 6 | | 5.68 |
| 263 | 431601 | 18, 20 (Normal Colon Tissue vs. Colon Metastasis) | 9 | 0 | 7.69 | |
| | | 08, 09 (Lung, High Met vs. Lung, Low Met) | 5 | 0 | 6.99 | |
| | | 15, 16 (Normal Colon vs. Colon Tumor Tissue) | 28 | 6 | 4.93 | |
| | | 15, 17 (Normal Colon Tissue vs. Colon Metastasis) | 28 | 9 | 3.34 | |
| 264 | 449891 | 15, 16 (Normal Colon vs. Colon Tumor Tissue) | 8 | 1 | 8.46 | |
| 265 | 556561 | 15, 17 (Normal Colon Tissue vs. Colon Metastasis) | 0 | 10 | | 9.32 |
| | | 16, 17 (Colon Tumor Tissue vs. Colon Metastasis) | 1 | 10 | | 9.85 |
| 266 | 554188 | 15, 16 (Normal Colon vs. Colon Tumor Tissue) | 6 | 0 | 6.34 | |
| 267 | 3247 | 01, 02 (Colon, High Met vs. Colon, Low Met) | 1 | 20 | | 21.68 |
| | | 16, 17 (Colon Tumor Tissue vs. Colon Metastasis) | 7 | 26 | | 3.66 |
| | | 15, 17 (Normal Colon Tissue vs. Colon Metastasis) | 4 | 26 | | 6.06 |
| 268 | 546705 | 16, 17 (Colon Tumor Tissue vs. Colon Metastasis) | 0 | 6 | | 5.91 |
| 269 | 560984 | 16, 17 (Colon Tumor Tissue vs. Colon Metastasis) | 0 | 6 | | 5.91 |
| 270 | 455820 | 16, 17 (Colon Tumor Tissue vs. Colon Metastasis) | 0 | 7 | | 6.89 |
| 271 | 643129 | 15, 17 (Normal Colon Tissue vs. Colon Metastasis) | 7 | 0 | 7.51 | |
| 272 | 454653 | 15, 17 (Normal Colon Tissue vs. Colon Metastasis) | 3 | 17 | | 5.28 |
| | | 16, 17 (Colon Tumor Tissue vs. Colon Metastasis) | 5 | 17 | | 3.35 |
| 273 | 456549 | 16, 17 (Colon Tumor Tissue vs. Colon Metastasis) | 1 | 8 | | 7.88 |
| 274 | 454806 | 15, 16 (Normal Colon vs. Colon Tumor Tissue) | 10 | 2 | 5.28 | |
| 275 | 724296 | 15, 16 (Normal Colon vs. Colon Tumor Tissue) | 0 | 128 | | 121.1 |
| | | 16, 17 (Colon Tumor Tissue vs. Colon Metastasis) | 128 | 0 | 129.99 | |
| 276 | 559280 | 15, 16 (Normal Colon vs. Colon Tumor Tissue) | 0 | 7 | | 6.62 |
| 277 | 171511 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 278 | 644242 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 279 | 734370 | 15, 16 (Normal Colon vs. Colon Tumor Tissue) | 0 | 11 | | 10.41 |
| | | 16, 17 (Colon Tumor Tissue vs. Colon Metastasis) | 11 | 0 | 11.17 | |
| 280 | 639459 | 15, 17 (Normal Colon Tissue vs. Colon Metastasis) | 11 | 0 | 11.81 | |
| | | 16, 17 (Colon Tumor Tissue vs. Colon Metastasis) | 9 | 0 | 9.14 | |
| 281 | 641679 | 15, 17 (Normal Colon Tissue vs. Colon Metastasis) | 8 | 0 | 8.59 | |
| | | 15, 16 (Normal Colon vs. Colon Tumor Tissue) | 8 | 0 | 8.46 | |
| 282 | 644611 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 283 | 550038 | 16, 17 (Colon Tumor Tissue vs. Colon Metastasis) | 0 | 7 | | 6.89 |
| | | 15, 17 (Normal Colon Tissue vs. Colon Metastasis) | 0 | 7 | | 6.52 |
| 284 | 452567 | 16, 17 (Colon Tumor Tissue vs. Colon Metastasis) | 3 | 14 | | 4.6 |
| | | 15, 16 (Normal Colon vs. Colon Tumor Tissue) | 21 | 3 | 7.4 | |
| 285 | 411113 | 16, 17 (Colon Tumor Tissue vs. Colon Metastasis) | 0 | 6 | | 5.91 |
| 286 | 650749 | 15, 17 (Normal Colon Tissue vs. Colon Metastasis) | 7 | 0 | 7.51 | |
| 287 | 558899 | 15, 16 (Normal Colon vs. Colon Tumor Tissue) | 6 | 0 | 6.34 | |
| 288 | 452986 | 15, 16 (Normal Colon vs. Colon Tumor Tissue) | 8 | 1 | 8.46 | |
| 289 | 393197 | 16, 17 (Colon Tumor Tissue vs. Colon Metastasis) | 7 | 0 | 7.11 | |
| 290 | 499424 | 15, 16 (Normal Colon vs. Colon Tumor Tissue) | 0 | 8 | | 7.57 |
| 291 | 21669 | 16, 17 (Colon Tumor Tissue vs. Colon Metastasis) | 0 | 7 | | 6.89 |
| 292 | 640590 | 15, 17 (Normal Colon Tissue vs. Colon Metastasis) | 9 | 0 | 9.66 | |
| | | 15, 16 (Normal Colon vs. Colon Tumor Tissue) | 9 | 1 | 9.51 | |
| 293 | 549936 | 16, 17 (Colon Tumor Tissue vs. Colon Metastasis) | 0 | 6 | | 5.91 |
| 294 | 448770 | 15, 17 (Normal Colon Tissue vs. Colon Metastasis) | 6 | 0 | 6.44 | |
| 295 | 559280 | 15, 16 (Normal Colon vs. Colon Tumor Tissue) | 0 | 7 | | 6.62 |
| 296 | 648934 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 297 | 452685 | 15, 16 (Normal Colon vs. Colon Tumor Tissue) | 15 | 5 | 3.17 | |
| 298 | 456549 | 16, 17 (Colon Tumor Tissue vs. Colon Metastasis) | 1 | 8 | | 7.88 |
| 299 | 446614 | 16, 17 (Colon Tumor Tissue vs. Colon Metastasis) | 7 | 0 | 7.11 | |
| 300 | 559280 | 15, 16 (Normal Colon vs. Colon Tumor Tissue) | 0 | 7 | | 6.62 |
| 301 | 446673 | 16, 17 (Colon Tumor Tissue vs. Colon Metastasis) | 8 | 1 | 8.12 | |
| 302 | 562550 | 16, 17 (Colon Tumor Tissue vs. Colon Metastasis) | 0 | 6 | | 5.91 |
| 303 | 467288 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 304 | 463824 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 305 | 393197 | 16, 17 (Colon Tumor Tissue vs. Colon Metastasis) | 7 | 0 | 7.11 | |
| 306 | 407077 | 15, 17 (Normal Colon Tissue vs. Colon Metastasis) | 0 | 8 | | 7.45 |
| | | 16, 17 (Colon Tumor Tissue vs. Colon Metastasis) | 1 | 8 | | 7.88 |
| 307 | 499424 | 15, 16 (Normal Colon vs. Colon Tumor Tissue) | 0 | 8 | | 7.57 |
| 308 | 554500 | 15, 16 (Normal Colon vs. Colon Tumor Tissue) | 6 | 0 | 6.34 | |
| 309 | 730143 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 310 | 595506 | 15, 16 (Normal Colon vs. Colon Tumor Tissue) | 0 | 6 | | 5.68 |
| 311 | 2334 | 15, 16 (Normal Colon vs. Colon Tumor Tissue) | 0 | 6 | | 5.68 |
| 312 | 647444 | 15, 17 (Normal Colon Tissue vs. Colon Metastasis) | 7 | 0 | 7.51 | |
| 313 | 380291 | 15, 16 (Normal Colon vs. Colon Tumor Tissue) | 6 | 0 | 6.34 | |
| 314 | 644849 | 15, 17 (Normal Colon Tissue vs. Colon Metastasis) | 8 | 0 | 8.59 | |
| 315 | 449457 | 15, 17 (Normal Colon Tissue vs. Colon Metastasis) | 0 | 7 | | 6.52 |
| 316 | 446673 | 16, 17 (Colon Tumor Tissue vs. Colon Metastasis) | 8 | 1 | 8.12 | |
| 317 | 549069 | 15, 16 (Normal Colon vs. Colon Tumor Tissue) | 11 | 56 | | 4.82 |
| | | 15, 17 (Normal Colon Tissue vs. Colon Metastasis) | 11 | 36 | | 3.05 |
| 318 | 728884 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 319 | 415058 | 16, 17 (Colon Tumor Tissue vs. Colon Metastasis) | 0 | 6 | | 5.91 |
| 320 | 553955 | 16, 17 (Colon Tumor Tissue vs. Colon Metastasis) | 0 | 6 | | 5.91 |
| 321 | 455820 | 16, 17 (Colon Tumor Tissue vs. Colon Metastasis) | 0 | 7 | | 6.89 |
| 322 | 549617 | 15, 16 (Normal Colon vs. Colon Tumor Tissue) | 0 | 6 | | 5.68 |
| 323 | 449831 | 16, 17 (Colon Tumor Tissue vs. Colon Metastasis) | 0 | 10 | | 9.85 |
| 324 | 451580 | 16, 17 (Colon Tumor Tissue vs. Colon Metastasis) | 0 | 6 | | 5.91 |
| 325 | 558899 | 15, 16 (Normal Colon vs. Colon Tumor Tissue) | 6 | 0 | 6.34 | |
| 326 | 562292 | 16, 17 (Colon Tumor Tissue vs. Colon Metastasis) | 0 | 6 | | 5.91 |
| 327 | 5830 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 328 | 8953 | 03, 04 (Breast, High Met vs. Breast, Non-Met) | 0 | 13 | | 13.32 |
| 329 | 8012 | 03, 04 (Breast, High Met vs. Breast, Non-Met) | 7 | 0 | 6.83 | |
| 330 | 185718 | 03, 04 (Breast, High Met vs. Breast, Non-Met) | 0 | 6 | | 6.15 |
| 331 | 729851 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 332 | 185597 | 03, 04 (Breast, High Met vs. Breast, Non-Met) | 0 | 8 | | 8.2 |
| 333 | 9887 | 03, 04 (Breast, High Met vs. Breast, Non-Met) | 7 | 0 | 6.83 | |
| 334 | 725825 | 15, 16 (Normal Colon vs. Colon Tumor Tissue) | 0 | 8 | | 7.57 |
| | | 16, 17 (Colon Tumor Tissue vs. Colon Metastasis) | 8 | 0 | 8.12 | |
| 335 | 6545 | 03, 04 (Breast, High Met vs. Breast, Non-Met) | 0 | 9 | | 9.22 |
| 336 | 21205 | 03, 04 (Breast, High Met vs. Breast, Non-Met) | 1 | 9 | | 9.22 |
| 337 | 8867 | 03, 04 (Breast, High Met vs. Breast, Non-Met) | 6 | 0 | 5.85 | |
| 338 | 729295 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 339 | 730430 | 15, 16 (Normal Colon vs. Colon Tumor Tissue) | 0 | 8 | | 7.57 |
| | | 16, 17 (Colon Tumor Tissue vs. Colon Metastasis) | 8 | 0 | 8.12 | |
| 340 | 7072 | 03, 04 (Breast, High Met vs. Breast, Non-Met) | 10 | 1 | 9.76 | |
| 341 | 730533 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 342 | 9121 | 01, 02 (Colon, High Met vs. Colon, Low Met) | 0 | 7 | | 7.59 |
| 343 | 11131 | 01, 02 (Colon, High Met vs. Colon, Low Met) | 0 | 6 | | 6.5 |
| 344 | 640116 | 15, 16 (Normal Colon vs. Colon Tumor Tissue) | 19 | 4 | 5.02 | |
| | | 15, 17 (Normal Colon Tissue vs. Colon Metastasis) | 19 | 0 | 20.39 | |
| 345 | 730282 | 16, 17 (Colon TumorTissue vs. Colon Metastasis) | 7 | 0 | 7.11 | |
| | | 15, 16 (Normal Colon vs. Colon Tumor Tissue) | 0 | 7 | | 6.62 |
| 346 | 550571 | 16, 17 (Colon Tumor Tissue vs. Colon Metastasis) | 0 | 6 | | 5.91 |
| 347 | 1183 | 01, 02 (Colon, High Met vs. Colon, Low Met) | 4 | 71 | | 19.24 |
| | | 03, 04 (Breast, High Met vs. Breast, Non-Met) | 32 | 15 | 2.08 | |
| 348 | 449437 | 15, 16 (Normal Colon vs. Colon Tumor Tissue) | 14 | 3 | 4.93 | |
| | | 16, 17 (Colon Tumor Tissue vs. Colon Metastasis) | 3 | 12 | | 3.94 |
| 349 | 8966 | 03, 04 (Breast, High Met vs. Breast, Non-Met) | 0 | 7 | | 7.17 |
| 350 | 6134 | 18, 20 (Normal Colon Tissue vs. Colon Metastasis) | 22 | 5 | 3.76 | |
| | | 15, 17 (Normal Colon Tissue vs. Colon Metastasis) | 142 | 40 | 3.81 | |
| | | 15, 16 (Normal Colon vs. Colon Tumor Tissue) | 142 | 42 | 3.57 | |
| | | 19, 20 (Colon Tumor Tissue vs. Colon Metastasis) | 22 | 5 | 3.29 | |
| 351 | 95700 | 03, 04 (Breast, High Met vs. Breast, Non-Met) | 1 | 21 | | 21.52 |
| 352 | 7066 | 01, 02 (Colon, High Met vs. Colon, Low Met) | 0 | 9 | | 9.76 |
| 353 | 648310 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 354 | 730059 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 355 | 736014 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 356 | 646577 | 16, 17 (Colon Tumor Tissue vs. Colon Metastasis) | 6 | 0 | 6.09 | |
| 357 | 732254 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| | | 23, 24 (Normal Lung Tissue vs. Lung Tumor Tissue) | 0 | 8 | | 7.92 |
| 358 | 7037 | 01, 02 (Colon, High Met vs. Colon, Low Met) | 0 | 9 | | 9.76 |
| 359 | 7037 | 01, 02 (Colon, High Met vs. Colon, Low Met) | 0 | 9 | | 9.76 |
| 360 | 6937 | 03, 04 (Breast, High Met vs. Breast, Non-Met) | 10 | 0 | 9.76 | |
| 361 | 7572 | 01, 02 (Colon, High Met vs. Colon, Low Met) | 0 | 8 | | 8.67 |
| 362 | 388085 | 15, 17 (Normal Colon Tissue vs. Colon Metastasis) | 9 | 0 | 9.66 | |
| | | 15, 16 (Normal Colon vs. Colon Tumor Tissue) | 9 | 0 | 9.51 | |
| 363 | 2676 | 01, 02 (Colon, High Met vs. Colon, Low Met) | 2 | 17 | | 9.22 |
| 364 | 639240 | 15, 16 (Normal Colon vs. Colon Tumor Tissue) | 9 | 0 | 9.51 | |
| | | 15, 17 (Normal Colon Tissue vs. Colon Metastasis) | 9 | 0 | 9.66 | |
| 365 | 650472 | 15, 17 (Normal Colon Tissue vs. Colon Metastasis) | 6 | 0 | 6.44 | |
| 366 | 727789 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 367 | 2495 | 03, 04 (Breast, High Met vs. Breast, Non-Met) | 27 | 5 | 5.27 | |
| | | 08, 09 (Lung, High Met vs. Lung, Low Met) | 13 | 2 | 9.08 | |
| 368 | 732254 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| | | 23, 24 (Normal Lung Tissue vs. Lung Tumor Tissue) | 0 | 8 | | 7.92 |
| 369 | 5268 | 01, 02 (Colon, High Met vs. Colon, Low Met) | 0 | 15 | | 16.26 |
| 370 | 11881 | 03, 04 (Breast, High Met vs. Breast, Non-Met) | 6 | 0 | 5.85 | |
| 371 | 448677 | 15, 16 (Normal Colon vs. Colon Tumor Tissue) | 11 | 1 | 11.63 | |
| 372 | 1876 | 03, 04 (Breast, High Met vs. Breast, Non-Met) | 15 | 3 | 4.88 | |
| 373 | 3441 | 01, 02 (Colon, High Met vs. Colon, Low Met) | 4 | 13 | | 3.52 |
| | | 03, 04 (Breast, High Met vs. Breast, Non-Met) | 8 | 0 | 7.81 | |
| 374 | 726134 | 15, 16 (Normal Colon vs. Colon Tumor Tissue) | 0 | 8 | | 7.57 |
| | | 16, 17 (Colon Tumor Tissue vs. Colon Metastasis) | 8 | 0 | 8.12 | |
| 375 | 9048 | 01, 02 (Colon, High Met vs. Colon, Low Met) | 7 | 0 | 6.46 | |
| 376 | 26489 | 03, 04 (Breast, High Met vs. Breast, Non-Met) | 1 | 8 | | 8.2 |
| 377 | 644205 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 378 | 468689 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 379 | 638971 | 15, 17 (Normal Colon Tissue vs. Colon Metastasis) | 8 | 0 | 8.59 | |
| 380 | 10274 | 03, 04 (Breast, High Met vs. Breast, Non-Met) | 0 | 7 | | 7.17 |
| 381 | 6725 | 03, 04 (Breast, High Met vs. Breast, Non-Met) | 10 | 0 | 9.76 | |
| 382 | 2488 | 15, 17 (Normal Colon Tissue vs. Colon Metastasis) | 0 | 8 | | 7.45 |
| 383 | 8366 | 21, 22 (Normal Prostate vs. Prostate Cancer) (Normal Prostate vs. | 2 | 15 | | 7.63 |
| | | Prostate Cancer) | | | | |
| | | 03, 04 (Breast, High Met vs. Breast, Non-Met) | 15 | 2 | 7.32 | |
| 384 | 502683 | 15, 16 (Normal Colon vs. Colon Tumor Tissue) | 6 | 0 | 6.34 | |
| 385 | 450914 | 15, 16 (Normal Colon vs. Colon Tumor Tissue) | 0 | 6 | | 5.68 |
| 386 | 21205 | 03, 04 (Breast, High Met vs. Breast, Non-Met) | 1 | 9 | | 9.22 |
| 387 | 644205 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 388 | 5268 | 01, 02 (Colon, High Met vs. Colon, Low Met) | 0 | 15 | | 16.26 |
| 389 | 8012 | 03, 04 (Breast, High Met vs. Breast, Non-Met) | 7 | 0 | 6.83 | |
| 390 | 11270 | 01, 02 (Colon, High Met vs. Colon, Low Met) | 0 | 6 | | 65 |
| 391 | 10924 | 03, 04 (Breast, High Met vs. Breast, Non-Met) | 6 | 0 | 5.85 | |
| 393 | 3650 | 03, 04 (Breast, High Met vs. Breast, Non-Met) | 10 | 2 | 4.88 | |
| 394 | 1655 | 01, 02 (Colon, High Met vs. Colon, Low Met) | 67 | 2 | 30.9 | |
| | | 21, 22 (Normal Prostate vs. Prostate Cancer) | 116 | 51 | 2.24 | |
| | | 03, 04 (Breast, High Met vs. Breast, Non-Met) | 0 | 58 | | 59.45 |
| 395 | 3275 | 03, 04 (Breast, High Met vs. Breast, Non-Met) | 8 | 0 | 7.81 | |
| 396 | 3355 | 03, 04 (Breast, High Met vs. Breast, Non-Met) | 21 | 7 | 2.93 | |
| 397 | 2078 | 03, 04 (Breast, High Met vs. Breast, Non-Met) | 11 | 2 | 5.37 | |
| 398 | 4809 | 08, 09 (Lung, High Met vs. Lung, Low Met) | 3 | 27 | | 6.44 |
| | | 01, 02 (Colon, High Met vs. Colon, Low Met) | 15 | 1 | 13.84 | |
| 399 | 6402 | 03, 04 (Breast, High Met vs. Breast, Non-Met) | 10 | 0 | 9.76 | |
| 400 | 555244 | 16, 17 (Colon Tumor Tissue vs. Colon Metastasis) | 0 | 11 | | 10.83 |
| 401 | 548965 | 16, 17 (Colon Tumor Tissue vs. Colon Metastasis) | 4 | 14 | | 3.45 |
| 402 | 4747 | 01, 02 (Colon, High Met vs. Colon, Low Met) | 2 | 10 | | 5.42 |
| 403 | 40208 | 03, 04 (Breast, High Met vs. Breast, Non-Met) | 0 | 6 | | 6.15 |
| 404 | 14596 | 08, 09 (Lung, High Met vs. Lung, Low Met) | 14 | 6 | 3.26 | |
| | | 03, 04 (Breast, High Met vs. Breast, Non-Met) | 1 | 17 | | 17.42 |
| 405 | 7110 | 03, 04 (Breast, High Met vs. Breast, Non-Met) | 4 | 19 | | 4.87 |
| 406 | 7110 | 03, 04 (Breast, High Met vs. Breast, Non-Met) | 4 | 19 | | 4.87 |
| 407 | 6592 | 03, 04 (Breast, High Met vs. Breast, Non-Met) | 12 | 1 | 11.71 | |
| 408 | 6455 | 03, 04 (Breast, High Met vs. Breast, Non-Met) | 6 | 0 | 5.85 | |
| 409 | 2738 | 03, 04 (Breast, High Met vs. Breast, Non-Met) | 15 | 4 | 3.66 | |
| 410 | 696 | 03, 04 (Breast, High Met vs. Breast, Non-Met) | 37 | 13 | 2.78 | |
| | | 01, 02 (Colon, High Met vs. Colon, Low Met) | 24 | 87 | | 3.93 |
| 411 | 379186 | 16, 17 (Colon Tumor Tissue vs. Colon Metastasis) | 6 | 0 | 6.09 | |
| 412 | 1588 | 03, 04 (Breast, High Met vs. Breast, Non-Met) | 15 | 95 | | 6.49 |
| | | 01, 02 (Colon, High Met vs. Colon, Low Met) | 45 | 19 | 2.18 | |
| 413 | 7007 | 01, 02 (Colon, High Met vs. Colon, Low Met) | 0 | 9 | | 9.76 |
| 414 | 9025 | 01, 02 (Colon, High Met vs. Colon, Low Met) | 7 | 0 | 6.46 | |
| 415 | 650749 | 15, 17 (Normal Colon Tissue vs. Colon Metastasis) | 7 | 0 | 7.51 | |
| 416 | 553158 | 16, 17 (Colon Tumor Tissue vs. Colon Metastasis) | 1 | 8 | | 7.88 |
| 417 | 641703 | 15, 17 (Normal Colon Tissue vs. Colon Metastasis) | 12 | 0 | 12.88 | |
| 418 | 833 | 15, 16 (Normal Colon vs. Colon Tumor Tissue) | 12 | 3 | 4.23 | |
| | | 08, 09 (Lung, High Met vs. Lung, Low Met) | 18 | 9 | 2.79 | |
| | | 03, 04 (Breast, High Met vs. Breast, Non-Met) | 16 | 3 | 5.2 | |
| 419 | 649259 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 420 | 451179 | 15, 17 (Normal Colon Tissue vs. Colon Metastasis) | 1 | 9 | | 8.38 |
| 421 | 9505 | 03, 04 (Breast, High Met vs. Breast, Non-Met) | 3 | 15 | | 5.12 |
| 422 | 736728 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 423 | 380412 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 424 | 642425 | 15, 17 (Normal Colon Tissue vs. Colon Metastasis) | 6 | 0 | 6.44 | |
| 425 | 405073 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 426 | 174250 | 16, 17 (Colon Tumor Tissue vs. Colon Metastasis) | 2 | 13 | | 6.4 |
| 427 | 726281 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 428 | 639029 | 15, 17 (Normal Colon Tissue vs. Colon Metastasis) | 6 | 0 | 6.44 | |
| 429 | 452245 | 16, 17 (Colon Tumor Tissue vs. Colon Metastasis) | 1 | 12 | | 11.82 |
| | | 15, 17 (Normal Colon Tissue vs. Colon Metastasis) | 1 | 12 | | 11.18 |
| 430 | 510254 | 15, 16 (Normal Colon vs. Colon Tumor Tissue) | 17 | 1 | 17.97 | |
| | | 16, 17 (Colon Tumor Tissue vs. Colon Metastasis) | 1 | 8 | | 7.88 |
| 431 | 642425 | 15, 17 (Normal Colon Tissue vs. Colon Metastasis) | 6 | 0 | 6.44 | |
| 432 | 51939 | 15, 16 (Normal Colon vs. Colon Tumor Tissue) | 28 | 3 | 9.87 | |
| | | 15, 17 (Normal Colon Tissue vs. Colon Metastasis) | 28 | 0 | 30.05 | |
| 433 | 7379 | 01, 02 (Colon, High Met vs. Colon, Low Met) | 0 | 6 | | 6.5 |
| 434 | 546632 | 16, 17 (Colon Tumor Tissue vs. Colon Metastasis) | 15 | 0 | 15.23 | |
| | | 23, 24 (Normal Lung Tissue vs. Lung Tumor Tissue) | 3 | 34 | | 11.22 |
| 435 | 734827 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 436 | 2554 | 03, 04 (Breast, High Met vs. Breast, Non-Met) | 20 | 7 | 2.79 | |
| 437 | 643285 | 16, 17 (Colon Tumor Tissue vs. Colon Metastasis) | 9 | 0 | 9.14 | |
| 438 | 448770 | 15, 17 (Normal Colon Tissue vs. Colon Metastasis) | 6 | 0 | 6.44 | |
| 439 | 375380 | 15, 17 (Normal Colon Tissue vs. Colon Metastasis) | 1 | 9 | | 8.38 |
| | | 16, 17 (Colon Tumor Tissue vs. Colon Metastasis) | 0 | 9 | | 8.86 |
| 440 | 726134 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 441 | 422687 | 16, 17 (Colon Tumor Tissue vs. Colon Metastasis) | 10 | 0 | 10.16 | |
| | | 15, 16 (Normal Colon vs. Colon Tumor Tissue) | 0 | 10 | | 9.46 |
| 442 | 448436 | 16, 17 (Colon Tumor Tissue vs. Colon Metastasis) | 20 | 2 | 10.16 | |
| | | 15, 16 (Normal Colon vs. Colon Tumor Tissue) | 4 | 20 | | 4.73 |
| 443 | 644893 | 16, 17 (Colon Tumor Tissue vs. Colon Metastasis) | 6 | 0 | 6.09 | |
| 444 | 559104 | 15, 16 (Normal Colon vs. Colon Tumor Tissue) | 0 | 6 | | 5.68 |
| 445 | 551172 | 15, 16 (Normal Colon vs. Colon Tumor Tissue) | 1 | 10 | | 9.46 |
| 446 | 724296 | 15, 16 (Normal Colon vs. Colon Tumor Tissue) | 0 | 128 | | 121.1 |
| | | 16, 17 (Colon Tumor Tissue vs. Colon Metastasis) | 128 | 0 | 129.99 | |
| 447 | 735936 | 15, 16 (Normal Colon vs. Colon Tumor Tissue) | 0 | 9 | | 8.51 |
| | | 16, 17 (Colon Tumor Tissue vs. Colon Metastasis) | 9 | 0 | 9.14 | |
| 448 | 556326 | 16, 17 (Colon Tumor Tissue vs. Colon Metastasis) | 0 | 8 | | 7.88 |
| 449 | 729699 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 450 | 550694 | 15, 17 (Normal Colon Tissue vs. Colon Metastasis) | 21 | 7 | 3.22 | |
| | | 15, 16 (Normal Colon vs. Colon Tumor Tissue) | 21 | 1 | 22.2 | |
| 451 | 734738 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 452 | 404502 | 15, 16 (Normal Colon vs. Colon Tumor Tissue) | 6 | 0 | 6.34 | |
| 453 | 554151 | 15, 17 (Normal Colon Tissue vs. Colon Metastasis) | 1 | 15 | | 13.97 |
| | | 15, 16 (Normal Colon vs. Colon Tumor Tissue) | 1 | 9 | | 8.51 |
| 454 | 649852 | 15, 17 (Normal Colon Tissue vs. Colon Metastasis) | 6 | 0 | 6.44 | |
| 455 | 734063 | 15, 16 (Normal Colon vs. Colon Tumour Tissue) | 0 | 7 | | 6.62 |
| | | 16, 17 (Colon Tumor Tissue vs. Colon Metastasis) | 7 | 0 | 7.11 | |
| 456 | 7279 | 01, 02 (Colon, High Met vs. Colon, Low Met) | 0 | 6 | | 6.5 |
| 457 | 2676 | 01, 02 (Colon, High Met vs. Colon, Low Met) | 2 | 17 | | 9.22 |
| 458 | 649148 | 15, 17 (Normal Colon Tissue vs. Colon Metastasis) | 8 | 0 | 8.59 | |
| 459 | 1953 | 03, 04 (Breast, High Met vs. Breast, Non-Met) | 4 | 48 | | 12.3 |
| | | 01, 02 (Colon, High Met vs. Colon, Low Met) | 39 | 13 | 2.77 | |
| 460 | 650108 | 15, 17 (Normal Colon Tissue vs. Colon Metastasis) | 9 | 0 | 9.66 | |
| 461 | 515350 | 15, 16 (Normal Colon vs. Colon Tumor Tissue) | 14 | 0 | 14.8 | |
| | | 15, 17 (Normal Colon Tissue vs. Colon Metastasis) | 14 | 3 | 5.01 | |
| 462 | 402494 | 15, 16 (Normal Colon vs. Colon Tumor Tissue) | 3 | 13 | | 4.1 |
| | | 16, 17 (Colon Tumor Tissue vs. Colon Metastasis) | 13 | 3 | 4.4 | |
| 463 | 649148 | 15, 17 (Normal Colon Tissue vs. Colon Metastasis) | 8 | 0 | 8.59 | |
| 464 | 833 | 03, 04 (Breast, High Met vs. Breast, Non-Met) | 16 | 3 | 5.2 | |
| | | 08, 09 (Lung, High Met vs. Lung, Low Met) | 18 | 9 | 2.79 | |
| | | 15, 16 (Normal Colon vs. Colon Tumor Tissue) | 12 | 3 | 4.23 | |
| 465 | 139730 | 03, 04 (Breast, High Met vs. Breast, Non-Met) | 0 | 9 | | 9.22 |
| 466 | 453079 | 16, 17 (Colon Tumor Tissue vs. Colon Metastasis) | 8 | 1 | 8.12 | |
| 467 | 546705 | 16, 17 (Colon Tumor Tissue vs. Colon Metastasis) | 0 | 6 | | 5.91 |
| 468 | 644903 | 15, 17 (Normal Colon Tissue vs. Colon Metastasis) | 7 | 0 | 7.51 | |
| 469 | 732254 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| | | 23, 24 (Normal Lung Tissue vs. Lung Tumor Tissue) | 0 | 8 | | 7.92 |
| 470 | 561180 | 15, 16 (Normal Colon vs. Colon Tumor Tissue) | 11 | 2 | 5.81 | |
| 471 | 732254 | 23, 24 (Normal Lung Tissue vs. Lung Tumor Tissue) | 0 | 8 | | 7.92 |
| | | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 472 | 449204 | 15, 17 (Normal Colon Tissue vs. Colon Metastasis) | 1 | 14 | | 13.04 |
| | | 16, 17 (Colon Tumor Tissue vs. Colon Metastasis) | 3 | 14 | | 4.6 |
| 473 | 185651 | 03, 04 (Breast, High Met vs. Breast, Non-Met) | 0 | 7 | | 7.17 |
| 474 | 639029 | 15, 17 (Normal Colon Tissue vs. Colon Metastasis) | 6 | 0 | 6.44 | |
| 475 | 452986 | 15, 16 (Normal Colon vs. Colon Tumor Tissue) | 8 | 1 | 8.46 | |
| 476 | 729779 | 15, 16 (Normal Colon vs. Colon Tumor Tissue) | 0 | 8 | | 7.57 |
| | | 16, 17 (Colon Tumor Tissue vs. Colon Metastasis) | 8 | 0 | 8.12 | |
| 477 | 646248 | 16, 17 (Colon Tumor Tissue vs. Colon Metastasis) | 6 | 0 | 6.09 | |
| 478 | 650448 | 15, 17 (Normal Colon Tissue vs. Colon Metastasis) | 7 | 0 | 7.51 | |
| 479 | 642049 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 480 | 728273 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 481 | 446139 | 15, 16 (Normal Colon vs. Colon Tumor Tissue) | 13 | 0 | 13.74 | |
| | | 18, 19 (Normal Colon Tissue vs. Colon Tumor) | 12 | 0 | 13.71 | |
| | | 15, 17 (Normal Colon Tissue vs. Colon Metastasis) | 13 | 0 | 13.95 | |
| | | 18, 20 (Normal Colon Tissue vs. Colon Metastasis) | 12 | 0 | 10.26 | |
| 482 | 2783 | 15, 16 (Normal Colon vs. Colon Tumor Tissue) | 6 | 0 | 6.34 | |
| | | 03, 04 (Breast, High Met vs. Breast, Non-Met) | 5 | 40 | | 8.2 |
| | | 01, 02 (Colon, High Met vs. Colon, Low Met) | 27 | 6 | 4.15 | |
| 483 | 642906 | 16, 17 (Colon Tumor Tissue vs. Colon Metastasis) | 7 | 0 | 7.11 | |
| 484 | 8332 | 03, 04 (Breast, High Met vs. Breast, Non-Met) | 6 | 0 | 5.85 | |
| 485 | 453470 | 15, 17 (Normal Colon Tissue vs. Colon Metastasis) | 12 | 1 | 12.88 | |
| | | 15, 16 (Normal Colon vs. Colon Tumor Tissue) | 12 | 1 | 12.68 | |
| 486 | 552277 | 15, 17 (Normal Colon Tissue vs. Colon Metastasis) | 0 | 8 | | 7.45 |
| 487 | 464029 | 16, 17 (Colon Tumor Tissue vs. Colon Metastasis) | 1 | 9 | | 8.86 |
| 489 | 649722 | 15, 17 (Normal Colon Tissue vs. Colon Metastasis) | 8 | 0 | 8.59 | |
| | | 16, 17 (Colon Tumor Tissue vs. Colon Metastasis) | 6 | 0 | 6.09 | |
| 490 | 612572 | 15, 16 (Normal Colon vs. Colon Tumor Tissue) | 6 | 0 | 6.34 | |
| 491 | 385980 | 16, 17 (Colon Tumor Tissue vs. Colon Metastasis) | 27 | 12 | 2.28 | |
| | | 15, 17 (Normal Colon Tissue vs. Colon Metastasis) | 2 | 12 | | 5.59 |
| | | 15, 16 (Normal Colon vs. Colon Tumor Tissue) | 2 | 27 | | 12.77 |
| 492 | 141185 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 493 | 463824 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 494 | 446139 | 15, 16 (Normal Colon vs. Colon Tumor Tissue) | 13 | 0 | 13.74 | |
| | | 15, 17 (Normal Colon Tissue vs. Colon Metastasis) | 13 | 0 | 13.95 | |
| | | 18, 19 (Normal Colon Tissue vs. Colon Tumor) | 12 | 0 | 13.71 | |
| | | 18, 20 (Normal Colon Tissue vs. Colon Metastasis) | 12 | 0 | 10.26 | |
| 495 | 725994 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 496 | 736679 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 497 | 551718 | 15, 17 (Normal Colon Tissue vs. Colon Metastasis) | 0 | 8 | | 7.45 |
| 498 | 640525 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 499 | 645210 | 15, 16 (Normal Colon vs. Colon Tumor Tissue) | 10 | 1 | 10.57 | |
| | | 15, 17 (Normal Colon Tissue vs. Colon Metastasis) | 10 | 0 | 10.73 | |
| 500 | 6567 | 03, 04 (Breast, High Met vs. Breast, Non-Met) | 10 | 0 | 9.76 | |
| 501 | 646146 | 16, 17 (Colon Tumor Tissue vs. Colon Metastasis) | 6 | 0 | 6.09 | |
| | | 15, 17 (Normal Colon Tissue vs. Colon Metastasis) | 8 | 0 | 8.59 | |
| 502 | 4934 | 01, 02 (Colon, High Met vs. Colon, Low Met) | 1 | 9 | | 9.76 |
| 503 | 450791 | 15, 16 (Normal Colon vs. Colon Tumor Tissue) | 18 | 6 | 3.17 | |
| | | 15, 17 (Normal Colon Tissue vs. Colon Metastasis) | 18 | 3 | 6.44 | |
| 504 | 227936 | 15, 16 (Normal Colon vs. Colon Tumor Tissue) | 0 | 8 | | 7.57 |
| 505 | 9436 | 16, 17 (Colon Tumor Tissue vs. Colon Metastasis) | 4 | 15 | | 3.69 |
| 506 | 2557 | 01, 02 (Colon, High Met vs. Colon, Low Met) | 23 | 8 | 2.65 | |
| 507 | 11356 | 01, 02 (Colon, High Met vs. Colon, Low Met) | 0 | 6 | | 6.5 |
| 508 | 7571 | 01, 02 (Colon, High Met vs. Colon, Low Met) | 0 | 8 | | 8.67 |
| 509 | 558116 | 16, 17 (Colon Tumor Tissue vs. Colon Metastasis) | 0 | 6 | | 5.91 |
| 510 | 216574 | 18, 20 (Normal Colon Tissue vs. Colon Metastasis) | 15 | 2 | 6.41 | |
| | | 15, 17 (Normal Colon Tissue vs. Colon Metastasis) | 107 | 29 | 3.96 | |
| | | 15, 16 (Normal Colon vs. Colon Tumor Tissue) | 107 | 29 | 3.9 | |
| 511 | 455145 | 15, 17 (Normal Colon Tissue vs. Colon Metastasis) | 6 | 0 | 6.44 | |
| 512 | 649148 | 15, 17 (Normal Colon Tissue vs. Colon Metastasis) | 8 | 0 | 8.59 | |
| 513 | 648996 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 514 | 304253 | 15, 16 (Normal Colon vs. Colon Tumor Tissue) | 0 | 6 | | 5.68 |
| 515 | 649717 | 16, 17 (Colon Tumor Tissue vs. Colon Metastasis) | 6 | 0 | 6.09 | |
| 516 | 5838 | 01, 02 (Colon, High Met vs. Colon, Low Met) | 1 | 9 | | 9.76 |
| 517 | 454050 | 15, 17 (Normal Colon Tissue vs. Colon Metastasis) | 8 | 1 | 8.59 | |
| 518 | 557903 | 15, 16 (Normal Colon vs. Colon Tumor Tissue) | 0 | 9 | | 8.51 |
| 519 | 1724 | 03, 04 (Breast, High Met vs. Breast, Non-Met) | 6 | 24 | | 4.1 |
| 520 | 734803 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 521 | 557948 | 16, 17 (Colon Tumor Tissue vs. Colon Metastasis) | 0 | 11 | | 10.83 |
| 522 | 5838 | 01, 02 (Colon, High Met vs. Colon, Low Met) | 1 | 9 | | 9.76 |
| 523 | 2334 | 15, 16 (Normal Colon vs. Colon Tumor Tissue) | 0 | 6 | | 5.68 |
| 524 | 450953 | 15, 17 (Normal Colon Tissue vs. Colon Metastasis) | 2 | 14 | | 6.52 |
| | | 16, 17 (Colon Tumor Tissue vs. Colon Metastasis) | 1 | 14 | | 13.79 |
| 525 | 4840 | 03, 04 (Breast, High Met vs. Breast, Non-Met) | 18 | 6 | 2.93 | |
| 526 | 728421 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 527 | 4747 | 01, 02 (Colon, High Met vs. Colon, Low Met) | 2 | 10 | | 5.42 |
| 528 | 648934 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 529 | 1787 | 01, 02 (Colon, High Met vs. Colon, Low Met) | 17 | 36 | | 2.3 |
| 530 | 558098 | 15, 16 (Normal Colon vs. Colon Tumor Tissue) | 6 | 0 | 6.34 | |
| 531 | 1655 | 03, 04 (Breast, High Met vs. Breast, Non-Met) | 0 | 58 | | 59.45 |
| | | 21, 22 (Normal Prostate vs. Prostate Cancer) | 116 | 51 | 2.24 | |
| | | 01, 02 (Colon, High Met vs. Colon, Low Met) | 67 | 2 | 30.9 | |
| 532 | 158601 | 03, 04 (Breast, High Met vs. Breast, Non-Met) | 1 | 10 | | 10.25 |
| 533 | 185486 | 03, 04 (Breast, High Met vs. Breast, Non-Met) | 0 | 13 | | 13.32 |
| 534 | 7110 | 03, 04 (Breast, High Met vs. Breast, Non-Met) | 4 | 19 | | 4.87 |
| 535 | 2543 | 01, 02 (Colon, High Met vs. Colon, Low Met) | 23 | 7 | 3.03 | |
| 536 | 115762 | 21, 22 (Normal Prostate vs. Prostate Cancer) | 16 | 5 | 3.15 | |
| | | 15, 16 (Normal Colon vs. Colon Tumor Tissue) | 0 | 6 | | 5.68 |
| | | 15, 17 (Normal Colon Tissue vs. Colon Metastasis) | 0 | 7 | | 6.52 |
| 537 | 696 | 01, 02 (Colon, High Met vs. Colon, Low Met) | 24 | 87 | | 3.93 |
| | | 03, 04 (Breast, High Met vs. Breast, Non-Met) | 37 | 13 | 2.78 | |
| 538 | 1948 | 03, 04 (Breast, High Met vs. Breast, Non-Met) | 37 | 15 | 2.41 | |
| 539 | 696 | 01, 02 (Colon, High Met vs. Colon, Low Met) | 24 | 87 | | 3.93 |
| | | 03, 04 (Breast, High Met vs. Breast, Non-Met) | 37 | 13 | 2.78 | |
| 540 | 696 | 03, 04 (Breast, High Met vs. Breast, Non-Met) | 37 | 13 | 2.78 | |
| | | 01, 02 (Colon, High Met vs. Colon, Low Met) | 24 | 87 | | 3.93 |
| 541 | 380477 | 15, 17 (Normal Colon Tissue vs. Colon Metastasis) | 7 | 0 | 7.51 | |
| 542 | 638799 | 15, 17 (Normal Colon Tissue vs. Colon Metastasis) | 10 | 0 | 10.73 | |
| 543 | 551982 | 16, 17 (Colon Tumor Tissue vs. Colon Metastasis) | 0 | 6 | | 5.91 |
| 544 | 551982 | 16, 17 (Colon Tumor Tissue vs. Colon Metastasis) | 0 | 6 | | 5.91 |
| 545 | 521840 | 15, 16 (Normal Colon vs. Colon Tumor Tissue) | 6 | 0 | 6.34 | |
| 546 | 561180 | 15, 16 (Normal Colon vs. Colon Tumor Tissue) | 11 | 2 | 5.81 | |
| 547 | 556245 | 15, 16 (Normal Colon vs. Colon Tumor Tissue) | 0 | 6 | | 5.68 |
| 548 | 449792 | 19, 20 (Colon Tumor Tissue vs. Colon Metastasis) | 0 | 5 | | 6.68 |
| | | 15, 16 (Normal Colon vs. Colon Tumor Tissue) | 3 | 13 | | 4.1 |
| 549 | 549722 | 16, 17 (Colon Tumor Tissue vs. Colon Metastasis) | 0 | 6 | | 5.91 |
| 550 | 612572 | 15, 16 (Normal Colon vs. Colon Tumor Tissue) | 6 | 0 | 6.34 | |
| 551 | 551235 | 16, 17 (Colon Tumor Tissue vs. Colon Metastasis) | 0 | 7 | | 6.89 |
| 552 | 449701 | 16, 17 (Colon Tumor Tissue vs. Colon Metastasis) | 17 | 1 | 17.26 | |
| | | 15, 16 (Normal Colon vs. Colon Tumor Tissue) | 1 | 17 | | 16.08 |
| 553 | 375380 | 16, 17 (Colon Tumor Tissue vs. Colon Metastasis) | 0 | 9 | | 8.86 |
| | | 15, 17 (Normal Colon Tissue vs. Colon Metastasis) | 1 | 9 | | 8.38 |
| 554 | 56940 | 15, 16 (Normal Colon vs. Colon Tumor Tissue) | 0 | 6 | | 5.68 |
| | | 15, 17 (Normal Colon Tissue vs. Colon Metastasis) | 0 | 8 | | 7.45 |
| 555 | 549160 | 15, 17 (Normal Colon Tissue vs. Colon Metastasis) | 0 | 7 | | 6.52 |
| 556 | 554151 | 15, 17 (Normal Colon Tissue vs. Colon Metastasis) | 1 | 15 | | 13.97 |
| | | 15, 16 (Normal Colon vs. Colon Tumor Tissue) | 1 | 9 | | 8.51 |
| 557 | 727331 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 558 | 551502 | 15, 16 (Normal Colon vs. Colon Tumor Tissue) | 22 | 7 | 3.32 | |
| | | 16, 17 (Colon Tumor Tissue vs. Colon Metastasis) | 7 | 19 | | 2.67 |
| 559 | 612572 | 15, 16 (Normal Colon vs. Colon Tumor Tissue) | 6 | 0 | 6.34 | |
| 560 | 701221 | 16, 17 (Colon Tumor Tissue vs. Colon Metastasis) | 7 | 0 | 7.11 | |
| 561 | 378041 | 16, 17 (Colon Tumor Tissue vs. Colon Metastasis) | 13 | 4 | 3.3 | |
| | | 15, 16 (Normal Colon vs. Colon Tumor Tissue) | 0 | 13 | | 12.3 |
| 562 | 503491 | 16, 17 (Colon Tumor Tissue vs. Colon Metastasis) | 6 | 0 | 6.09 | |
| 563 | 452833 | 15, 17 (Normal Colon Tissue vs. Colon Metastasis) | 0 | 7 | | 6.52 |
| 564 | 640974 | 15, 17 (Normal Colon Tissue vs. Colon Metastasis) | 9 | 0 | 9.66 | |
| 565 | 735326 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 566 | 555944 | 16, 17 (Colon Tumor Tissue vs. Colon Metastasis) | 0 | 6 | | 5.91 |
| 567 | 447532 | 15, 16 (Normal Colon vs. Colon Tumor Tissue) | 11 | 0 | 11.63 | |
| | | 15, 17 (Normal Colon Tissue vs. Colon Metastasis) | 11 | 1 | 11.81 | |
| 568 | 455598 | 16, 17 (Colon Tumor Tissue vs. Colon Metastasis) | 0 | 6 | | 5.91 |
| 569 | 555734 | 15, 17 (Normal Colon Tissue vs. Colon Metastasis) | 0 | 10 | | 9.32 |
| | | 16, 17 (Colon Tumor Tissue vs. Colon Metastasis) | 0 | 10 | | 9.85 |
| 570 | 446663 | 16, 17 (Colon Tumor Tissue vs. Colon Metastasis) | 6 | 32 | | 5.25 |
| | | 18, 20 (Normal Colon Tissue vs. Colon Metastasis) | 1 | 8 | | 9.36 |
| | | 19, 20 (Colon Tumor Tissue vs. Colon Metastasis) | 0 | 8 | | 10.7 |
| | | 15, 17 (Normal Colon Tissue vs. Colon Metastasis) | 14 | 32 | | 2.13 |
| 571 | 449862 | 15, 17 (Normal Colon Tissue vs. Colon Metastasis) | 0 | 7 | | 6.52 |
| 572 | 549591 | 16, 17 (Colon Tumor Tissue vs. Colon Metastasis) | 7 | 24 | | 3.38 |
| | | 15, 17 (Normal Colon Tissue vs. Colon Metastasis) | 2 | 24 | | 11.18 |
| 573 | 553877 | 16, 17 (Colon Tumor Tissue vs. Colon Metastasis) | 1 | 8 | | 7.88 |
| 574 | 553501 | 15, 16 (Normal Colon vs. Colon Tumor Tissue) | 14 | 1 | 14.8 | |
| | | 15, 17 (Normal Colon Tissue vs. Colon Metastasis) | 14 | 3 | 5.01 | |
| 575 | 1905 | 01, 02 (Colon, High Met vs. Colon, Low Met) | 7 | 21 | | 3.25 |
| | | 15, 17 (Normal Colon Tissue vs. Colon Metastasis) | 0 | 7 | | 6.52 |
| 576 | 446599 | 15, 16 (Normal Colon vs. Colon Tumor Tissue) | 13 | 2 | 6.87 | |
| 577 | 559409 | 16, 17 (Colon Tumor Tissue vs. Colon Metastasis) | 0 | 6 | | 5.91 |
| 578 | 551982 | 16, 17 (Colon Tumor Tissue vs. Colon Metastasis) | 0 | 6 | | 5.91 |
| 579 | 559057 | 15, 17 (Normal Colon Tissue vs. Colon Metastasis) | 0 | 7 | | 6.52 |
| 580 | 446760 | 16, 17 (Colon Tumor Tissue vs. Colon Metastasis) | 22 | 7 | 3.19 | |
| 581 | 551502 | 16, 17 (Colon Tumor Tissue vs. Colon Metastasis) | 7 | 19 | | 2.67 |
| | | 15, 16 (Normal Colon vs. Colon Tumor Tissue) | 22 | 7 | 3.32 | |
| 582 | 446531 | 16, 17 (Colon Tumor Tissue vs. Colon Metastasis) | 0 | 6 | | 5.91 |
| 583 | 506744 | 15, 16 (Normal Colon vs. Colon Tumor Tissue) | 7 | 0 | 7.4 | |
| | | 16, 17 (Colon Tumor Tissue vs. Colon Metastasis) | 0 | 8 | | 7.88 |
| 584 | 401849 | 15, 16 (Normal Colon vs. Colon Tumor Tissue) | 7 | 0 | 7.4 | |
| | | 16, 17 (Colon Tumor Tissue vs. Colon Metastasis) | 0 | 15 | | 14.77 |
| 585 | 453848 | 15, 16 (Normal Colon vs. Colon Tumor Tissue) | 2 | 14 | | 6.62 |
| 586 | 456764 | 16, 17 (Colon Tumor Tissue vs. Colon Metastasis) | 10 | 1 | 10.16 | |
| | | 15, 17 (Normal Colon Tissue vs. Colon Metastasis) | 14 | 1 | 15.03 | |
| 587 | 446371 | 15, 16 (Normal Colon vs. Colon Tumor Tissue) | 6 | 0 | 6.34 | |
| 588 | 406413 | 16, 17 (Colon Tumor Tissue vs. Colon Metastasis) | 1 | 9 | | 8.86 |
| 589 | 555103 | 15, 17 (Normal Colon Tissue vs. Colon Metastasis) | 0 | 7 | | 6.52 |
| | | 23, 24 (Normal Lung Tissue vs. Lung Tumor Tissue) | 0 | 6 | | 5.94 |
| 590 | 735292 | 15, 16 (Normal Colon vs. Colon Tumor Tissue) | 0 | 9 | | 8.51 |
| | | 16, 17 (Colon Tumor Tissue vs. Colon Metastasis) | 9 | 0 | 9.14 | |
| 591 | 558534 | 16, 17 (Colon Tumor Tissue vs. Colon Metastasis) | 0 | 7 | | 6.89 |
| 592 | 727181 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 593 | 551117 | 16, 17 (Colon Tumor Tissue vs. Colon Metastasis) | 0 | 6 | | 5.91 |
| 594 | 464040 | 15, 16 (Normal Colon vs. Colon Tumor Tissue) | 6 | 0 | 6.34 | |
| 595 | 446371 | 15, 16 (Normal Colon vs. Colon Tumor Tissue) | 6 | 0 | 6.34 | |
| 596 | 728408 | 16, 17 (Colon Tumor Tissue vs. Colon Metastasis) | 12 | 0 | 12.19 | |
| | | 15, 16 (Normal Colon vs. Colon Tumor Tissue) | 0 | 12 | | 11.35 |
| 597 | 649259 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 598 | 15414 | 15, 17 (Normal Colon Tissue vs. Colon Metastasis) | 1 | 9 | | 8.38 |
| 599 | 639240 | 15, 17 (Normal Colon Tissue vs. Colon Metastasis) | 9 | 0 | 9.66 | |
| | | 15, 16 (Normal Colon vs. Colon Tumor Tissue) | 9 | 0 | 9.51 | |
| 600 | 549722 | 16, 17 (Colon Tumor Tissue vs. Colon Metastasis) | 0 | 6 | | 5.91 |
| 601 | 561499 | 16, 17 (Colon Tumor Tissue vs. Colon Metastasis) | 1 | 8 | | 7.88 |
| | | 15, 16 (Normal Colon vs. Colon Tumor Tissue) | 8 | 1 | 8.46 | |
| 602 | 639029 | 15, 17 (Normal Colon Tissue vs. Colon Metastasis) | 6 | 0 | 6.44 | |
| 603 | 449512 | 15, 17 (Normal Colon Tissue vs. Colon Metastasis) | 4 | 14 | | 3.26 |
| | | 16, 17 (Colon Tumor Tissue vs. Colon Metastasis) | 1 | 14 | | 13.79 |
| 604 | 446987 | 15, 17 (Normal Colon Tissue vs. Colon Metastasis) | 10 | 0 | 10.73 | |
| 605 | 466302 | 15, 16 (Normal Colon vs. Colon Tumor Tissue) | 9 | 1 | 9.51 | |
| 606 | 553802 | 15, 17 (Normal Colon Tissue vs. Colon Metastasis) | 1 | 9 | | 8.38 |
| 607 | 639662 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 608 | 551527 | 15, 17 (Normal Colon Tissue vs. Colon Metastasis) | 0 | 7 | | 6.52 |
| 609 | 730389 | 16, 17 (Colon Tumor Tissue vs. Colon Metastasis) | 9 | 0 | 9.14 | |
| | | 15, 16 (Normal Colon vs. Colon Tumor Tissue) | 0 | 9 | | 8.51 |
| 610 | 640974 | 15, 17 (Normal Colon Tissue vs. Colon Metastasis) | 9 | 0 | 9.66 | |
| 611 | 417155 | 15, 16 (Normal Colon vs. Colon Tumor Tissue) | 6 | 0 | 6.34 | |
| 612 | 417155 | 15, 16 (Normal Colon vs. Colon Tumor Tissue) | 6 | 0 | 6.34 | |
| 613 | 451784 | 16, 17 (Colon Tumor Tissue vs. Colon Metastasis) | 0 | 7 | | 6.89 |
| 614 | 649152 | 15, 17 (Normal Colon Tissue vs. Colon Metastasis) | 6 | 0 | 6.44 | |
| 615 | 450867 | 15, 16 (Normal Colon vs. Colon Tumor Tissue) | 7 | 0 | 7.4 | |
| 616 | 143436 | 15, 17 (Normal Colon Tissue vs. Colon Metastasis) | 21 | 9 | 2.5 | |
| 617 | 549395 | 16, 17 (Colon Tumor Tissue vs. Colon Metastasis) | 1 | 13 | | 12.8 |
| | | 15, 17 (Normal Colon Tissue vs. Colon Metastasis) | 2 | 13 | | 6.06 |
| 618 | 639273 | 15, 16 (Normal Colon vs. Colon Tumor Tissue) | 9 | 0 | 9.51 | |
| | | 15, 17 (Normal Colon Tissue vs. Colon Metastasis) | 9 | 0 | 9.66 | |
| 619 | 506744 | 15, 16 (Normal Colon vs. Colon Tumor Tissue) | 7 | 0 | 7.4 | |
| | | 16, 17 (Colon Tumor Tissue vs. Colon Metastasis) | 0 | 8 | | 7.88 |
| 620 | 736595 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 621 | 230995 | 15, 16 (Normal Colon vs. Colon Tumor Tissue) | 0 | 7 | | 6.62 |
| 622 | 451784 | 16, 17 (Colon Tumor Tissue vs. Colon Metastasis) | 0 | 7 | | 6.89 |
| 623 | 226324 | 16, 17 (Colon Tumor Tissue vs. Colon Metastasis) | 0 | 6 | | 5.91 |
| 624 | 449617 | 16, 17 (Colon Tumor Tissue vs. Colon Metastasis) | 7 | 18 | | 2.53 |
| 625 | 451092 | 16, 17 (Colon Tumor Tissue vs. Colon Metastasis) | 1 | 9 | | 8.86 |
| 626 | 546642 | 16, 17 (Colon Tumor Tissue vs. Colon Metastasis) | 11 | 0 | 11.17 | |
| 627 | 553736 | 16, 17 (Colon Tumor Tissue vs. Colon Metastasis) | 2 | 10 | | 4.92 |
| 628 | 394413 | 15, 16 (Normal Colon vs. Colon Tumor Tissue) | 113 | 3 | 39.81 | |
| | | 18, 20 (Normal Colon Tissue vs. Colon Metastasis) | 21 | 0 | 17.95 | |
| | | 18, 19 (Normal Colon Tissue vs. Colon Tumor) | 21 | 2 | 12 | |
| | | 15, 17 (Normal Colon Tissue vs. Colon Metastasis) | 113 | 0 | 121.29 | |
| 629 | 556326 | 16, 17 (Colon Tumor Tissue vs. Colon Metastasis) | 0 | 8 | | 7.88 |
| 630 | 448606 | 15, 17 (Normal Colon Tissue vs. Colon Metastasis) | 3 | 20 | | 6.21 |
| 631 | 394413 | 15, 17 (Normal Colon Tissue vs. Colon Metastasis) | 113 | 0 | 121.29 | |
| | | 18, 19 (Normal Colon Tissue vs. Colon Tumor) | 21 | 2 | 12 | |
| | | 15, 16 (Normal Colon vs. Colon Tumor Tissue) | 113 | 3 | 39.81 | |
| | | 18, 20 (Normal Colon Tissue vs. Colon Metastasis) | 21 | 0 | 17.95 | |
| 632 | 645633 | 15, 17 (Normal Colon Tissue vs. Colon Metastasis) | 7 | 0 | 7.51 | |
| 633 | 551634 | 16, 17 (Colon Tumor Tissue vs. Colon Metastasis) | 0 | 6 | | 5.91 |
| 634 | 556326 | 16, 17 (Colon Tumor Tissue vs. Colon Metastasis) | 0 | 8 | | 7.88 |
| 635 | 540787 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 636 | 648872 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 637 | 643804 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 638 | 446139 | 15, 17 (Normal Colon Tissue vs. Colon Metastasis) | 13 | 0 | 13.95 | |
| | | 18, 19 (Normal Colon Tissue vs. Colon Tumor) | 12 | 0 | 13.71 | |
| | | 18, 20 (Normal Colon Tissue vs. Colon Metastasis) | 12 | 0 | 10.26 | |
| | | 15, 16 (Normal Colon vs. Colon Tumor Tissue) | 13 | 0 | 13.74 | |
| 639 | 640356 | 15, 17 (Normal Colon Tissue vs. Colon Metastasis) | 10 | 0 | 10.73 | |
| 640 | 379186 | 16, 17 (Colon Tumor Tissue vs. Colon Metastasis) | 6 | 0 | 6.09 | |
| 641 | 454927 | 16, 17 (Colon Tumor Tissue vs. Colon Metastasis) | 3 | 14 | | 4.6 |
| 642 | 401849 | 16, 17 (Colon Tumor Tissue vs. Colon Metastasis) | 0 | 15 | | 14.77 |
| | | 15, 16 (Normal Colon vs. Colon Tumor Tissue) | 7 | 0 | 7.4 | |
| 643 | 452414 | 15, 16 (Normal Colon vs. Colon Tumor Tissue) | 17 | 0 | 17.97 | |
| | | 15, 17 (Normal Colon Tissue vs. Colon Metastasis) | 17 | 0 | 18.25 | |
| 644 | 446789 | 15, 16 (Normal Colon vs. Colon Tumor Tissue) | 16 | 5 | 3.38 | |
| 645 | 189561 | 15, 16 (Normal Colon vs. Colon Tumor Tissue) | 0 | 9 | | 8.51 |
| | | 08, 09 (Lung, High Met vs. Lung, Low Met) | 1 | 14 | | 10.02 |
| | | 15, 17 (Normal Colon Tissue vs. Colon Metastasis) | 0 | 9 | | 8.38 |
| 646 | 640323 | 15, 17 (Normal Colon Tissue vs. Colon Metastasis) | 8 | 0 | 8.59 | |
| | | 16, 17 (Colon Tumor Tissue vs. Colon Metastasis) | 6 | 0 | 6.09 | |
| 647 | 558116 | 16, 17 (Colon Tumor Tissue vs. Colon Metastasis) | 0 | 6 | | 5.91 |
| 648 | 468109 | 15, 17 (Normal Colon Tissue vs. Colon Metastasis) | 6 | 0 | 6.44 | |
| 649 | 481441 | 16, 17 (Colon Tumor Tissue vs. Colon Metastasis) | 12 | 0 | 12.19 | |
| 650 | 449956 | 16, 17 (Colon Tumor Tissue vs. Colon Metastasis) | 10 | 1 | 10.16 | |
| 651 | 727224 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 652 | 551907 | 15, 16 (Normal Colon vs. Colon Tumor Tissue) | 7 | 0 | 7.4 | |
| 653 | 447532 | 15, 16 (Normal Colon vs. Colon Tumor Tissue) | 11 | 0 | 11.63 | |
| | | 15, 17 (Normal Colon Tissue vs. Colon Metastasis) | 11 | 1 | 11.81 | |
| 654 | 447532 | 15, 17 (Normal Colon Tissue vs. Colon Metastasis) | 11 | 1 | 11.81 | |
| | | 15, 16 (Normal Colon vs. Colon Tumor Tissue) | 11 | 0 | 11.63 | |
| 655 | 558454 | 15, 17 (Normal Colon Tissue vs. Colon Metastasis) | 0 | 7 | | 6.52 |
| 656 | 502683 | 15, 16 (Normal Colon vs. Colon Tumor Tissue) | 6 | 0 | 6.34 | |
| 657 | 446909 | 16, 17 (Colon Tumor Tissue vs. Colon Metastasis) | 2 | 12 | | 5.91 |
| 658 | 452506 | 15, 16 (Normal Colon vs. Colon Tumor Tissue) | 7 | 0 | 7.4 | |
| | | 16, 17 (Colon Tumor Tissue vs. Colon Metastasis) | 0 | 6 | | 5.91 |
| 659 | 449792 | 19, 20 (Colon Tumor Tissue vs. Colon Metastasis) | 0 | 5 | | 6.68 |
| | | 15, 16 (Normal Colon vs. Colon Tumor Tissue) | 3 | 13 | | 4.1 |
| 660 | 549395 | 15, 17 (Normal Colon Tissue vs. Colon Metastasis) | 2 | 13 | | 6.06 |
| | | 16, 17 (Colon Tumor Tissue vs. Colon Metastasis) | 1 | 13 | | 12.8 |
| 661 | 234653 | 16, 17 (Colon Tumor Tissue vs. Colon Metastasis) | 6 | 18 | | 2.95 |
| 662 | 453911 | 15, 16 (Normal Colon vs. Colon Tumor Tissue) | 0 | 6 | | 5.68 |
| 663 | 452071 | 18, 20 (Normal Colon Tissue vs. Colon Metastasis) | 14 | 0 | 11.97 | |
| | | 15, 16 (Normal Colon vs. Colon Tumor Tissue) | 109 | 1 | 115.21 | |
| | | 15, 17 (Normal Colon Tissue vs. Colon Metastasis) | 109 | 0 | 117 | |
| | | 18, 19 (Normal Colon Tissue vs. Colon Tumor) | 14 | 0 | 16 | |
| 664 | 451032 | 15, 16 (Normal Colon vs. Colon Tumor Tissue) | 6 | 0 | 6.34 | |
| 665 | 446680 | 15, 17 (Normal Colon Tissue vs. Colon Metastasis) | 29 | 84 | | 2.7 |
| | | 23, 24 (Normal Lung Tissue vs. Lung Tumor Tissue) | 40 | 94 | | 2.33 |
| 666 | 641884 | 16, 17 (Colon Tumor Tissue vs. Colon Metastasis) | 7 | 0 | 7.11 | |
| | | 15, 17 (Normal Colon Tissue vs. Colon Metastasis) | 8 | 0 | 8.59 | |
| 667 | 452800 | 15, 17 (Normal Colon Tissue vs. Colon Metastasis) | 3 | 13 | | 4.04 |
| 668 | 461835 | 15, 16 (Normal Colon vs. Colon Tumor Tissue) | 7 | 0 | 7.4 | |
| 669 | 548965 | 16, 17 (Colon Tumor Tissue vs. Colon Metastasis) | 4 | 14 | | 3.45 |
| 670 | 734793 | 15, 16 (Normal Colon vs. Colon Tumor Tissue) | 0 | 9 | | 8.51 |
| | | 16, 17 (Colon Tumor Tissue vs. Colon Metastasis) | 9 | 0 | 9.14 | |
| 671 | 539955 | 23, 24 (Normal Lung Tissue vs. Lung Tumor Tissue) | 3 | 47 | | 15.51 |
| | | 16, 17 (Colon Tumor Tissue vs. Colon Metastasis) | 24 | 0 | 24.37 | |
| | | 15, 17 (Normal Colon Tissue vs. Colon Metastasis) | 11 | 0 | 11.81 | |
| 672 | 561892 | 15, 17 (Normal Colon Tissue vs. Colon Metastasis) | 0 | 7 | | 6.52 |
| 673 | 562292 | 16, 17 (Colon Tumor Tissue vs. Colon Metastasis) | 0 | 6 | | 5.91 |
| 674 | 420686 | 16, 17 (Colon Tumor Tissue vs. Colon Metastasis) | 0 | 8 | | 7.88 |
| 675 | 9436 | 16, 17 (Colon Tumor Tissue vs. Colon Metastasis) | 4 | 15 | | 3.69 |
| 676 | 1013 | 01, 02 (Colon, High Met vs. Colon, Low Met) | 40 | 84 | | 2.28 |
| | | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 677 | 412364 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 678 | 44424 | 08, 09 (Lung, High Met vs. Lung, Low Met) | 4 | 40 | | 7.16 |
| | | 15, 17 (Normal Colon Tissue vs. Colon Metastasis) | 25 | 90 | | 3.35 |
| | | 16, 17 (Colon Tumor Tissue vs. Colon Metastasis) | 41 | 90 | | 2.16 |
| 679 | 394413 | 15, 17 (Normal Colon Tissue vs. Colon Metastasis) | 113 | 0 | 121.29 | |
| | | 18, 19 (Normal Colon Tissue vs. Colon Tumor) | 21 | 2 | 12 | |
| | | 15, 16 (Normal Colon vs. Colon Tumor Tissue) | 113 | 3 | 39.81 | |
| | | 18, 20 (Normal Colon Tissue vs. Colon Metastasis) | 21 | 0 | 17.95 | |
| 680 | 449617 | 16, 17 (Colon Tumor Tissue vs. Colon Metastasis) | 7 | 18 | | 2.53 |
| 681 | 455032 | 15, 16 (Normal Colon vs. Colon Tumor Tissue) | 0 | 6 | | 5.68 |
| 682 | 185400 | 03, 04 (Breast, High Met vs. Breast, Non-Met) | 1 | 62 | | 63.55 |
| | | 16, 17 (Colon Tumor Tissue vs. Colon Metastasis) | 1 | 9 | | 8.86 |
| 683 | 453911 | 15, 16 (Normal Colon vs. Colon Tumor Tissue) | 0 | 6 | | 5.68 |
| 684 | 650297 | 15, 17 (Normal Colon Tissue vs. Colon Metastasis) | 6 | 0 | 6.44 | |
| 685 | 185400 | 03, 04 (Breast, High Met vs. Breast, Non-Met) | 1 | 62 | | 63.55 |
| | | 16, 17 (Colon Tumor Tissue vs. Colon Metastasis) | 1 | 9 | | 8.86 |
| 686 | 449512 | 15, 17 (Normal Colon Tissue vs. Colon Metastasis) | 4 | 14 | | 3.26 |
| | | 16, 17 (Colon Tumor Tissue vs. Colon Metastasis) | 1 | 14 | | 13.79 |
| 687 | 44424 | 16, 17 (Colon Tumor Tissue vs. Colon Metastasis) | 41 | 90 | | 2.16 |
| | | 15, 17 (Normal Colon Tissue vs. Colon Metastasis) | 25 | 90 | | 3.35 |
| | | 08, 09 (Lung, High Met vs. Lung, Low Met) | 4 | 40 | | 7.16 |
| 688 | 556216 | 16, 17 (Colon Tumor Tissue vs. Colon Metastasis) | 0 | 7 | | 6.89 |
| | | 15, 17 (Normal Colon Tissue vs. Colon Metastasis) | 0 | 7 | | 6.52 |
| 689 | 448677 | 15, 16 (Normal Colon vs. Colon Tumor Tissue) | 11 | 1 | 11.63 | |
| 690 | 375380 | 15, 17 (Normal Colon Tissue vs. Colon Metastasis) | 1 | 9 | | 8.38 |
| | | 16, 17 (Colon Tumor Tissue vs. Colon Metastasis) | 0 | 9 | | 8.86 |
| 691 | 379341 | 15, 17 (Normal Colon Tissue vs. Colon Metastasis) | 3 | 14 | | 4.35 |
| | | 08, 09 (Lung, High Met vs. Lung, Low Met) | 2 | 21 | | 7.51 |
| 692 | 376988 | 16, 17 (Colon Tumor Tissue vs. Colon Metastasis) | 1 | 8 | | 7.88 |
| 693 | 559806 | 16, 17 (Colon Tumor Tissue vs. Colon Metastasis) | 0 | 6 | | 5.91 |
| 694 | 550195 | 16, 17 (Colon Tumor Tissue vs. Colon Metastasis) | 0 | 11 | | 10.83 |
| | | 15, 17 (Normal Colon Tissue vs. Colon Metastasis) | 2 | 11 | | 5.12 |
| 695 | 562221 | 15, 16 (Normal Colon vs. Colon Tumor Tissue) | 8 | 0 | 8.46 | |
| 696 | 211 | 01, 02 (Colon, High Met vs. Colon, Low Met) | 109 | 206 | | 2.05 |
| | | 03, 04 (Breast, High Met vs. Breast, Non-Met) | 121 | 43 | 2.75 | |
| 697 | 6751 | 03, 04 (Breast, High Met vs. Breast, Non-Met) | 0 | 11 | | 11.27 |
| | | 08, 09 (Lung, High Met vs. Lung, Low Met) | 5 | 0 | 6.99 | |
| 698 | 6751 | 08, 09 (Lung, High Met vs. Lung, Low Met) | 5 | 0 | 6.99 | |
| | | 03, 04 (Breast, High Met vs. Breast, Non-Met) | 0 | 11 | | 11.27 |
| 700 | 2883 | 03, 04 (Breast, High Met vs. Breast, Non-Met) | 9 | 21 | | 2.39 |
| 701 | 9784 | 03, 04 (Breast, High Met vs. Breast, Non-Met) | 0 | 6 | | 6.15 |
| 702 | 649722 | 15, 17 (Normal Colon Tissue vs. Colon Metastasis) | 8 | 0 | 8.59 | |
| | | 16, 17 (Colon Tumor Tissue vs. Colon Metastasis) | 6 | 0 | 6.09 | |
| 704 | 10340 | 03, 04 (Breast, High Met vs. Breast, Non-Met) | 6 | 0 | 5.85 | |
| 705 | 1649 | 03, 04 (Breast, High Met vs. Breast, Non-Met) | 15 | 1 | 14.63 | |
| 706 | 4325 | 03, 04 (Breast, High Met vs. Breast, Non-Met) | 7 | 0 | 6.83 | |
| 707 | 10882 | 03, 04 (Breast, High Met vs. Breast, Non-Met) | 6 | 20 | | 3.42 |
| 708 | 10342 | 03, 04 (Breast, High Met vs. Breast, Non-Met) | 7 | 0 | 6.83 | |
| 709 | 6474 | 01, 02 (Colon, High Met vs. Colon, Low Met) | 0 | 6 | | 6.5 |
| 710 | 10340 | 03, 04 (Breast, High Met vs. Breast, Non-Met) | 6 | 0 | 5.85 | |
| 711 | 734723 | 16, 17 (Colon Tumor Tissue vs. Colon Metastasis) | 9 | 0 | 9.14 | |
| | | 15, 16 (Normal Colon vs. Colon Tumor Tissue) | 0 | 9 | | 8.51 |
| 712 | 452142 | 15, 16 (Normal Colon vs. Colon Tumor Tissue) | 1 | 10 | | 9.46 |
| | | 16, 17 (Colon Tumor Tissue vs. Colon Metastasis) | 10 | 2 | 5.08 | |
| 713 | 185432 | 03, 04 (Breast, High Met vs. Breast, Non-Met) | 0 | 26 | | 26.65 |
| 714 | 11456 | 01, 02 (Colon, High Met vs. Colon, Low Met) | 0 | 6 | | 6.5 |
| 715 | 508892 | 15, 17 (Normal Colon Tissue vs. Colon Metastasis) | 1 | 10 | | 9.32 |
| | | 16, 17 (Colon Tumor Tissue vs. Colon Metastasis) | 2 | 10 | | 4.92 |
| 716 | 67 | 03, 04 (Breast, High Met vs. Breast, Non-Met) | 23 | 0 | 22.44 | |
| | | 01, 02 (Colon, High Met vs. Colon, Low Met) | 0 | 32 | | 34.69 |
| 717 | 2636 | 08, 09 (Lung, High Met vs. Lung, Low Met) | 7 | 1 | 9.78 | |
| | | 01, 02 (Colon, High Met vs. Colon, Low Met) | 8 | 19 | | 2.57 |
| 718 | 735028 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 719 | 1924 | 01, 02 (Colon, High Met vs. Colon, Low Met) | 8 | 21 | | 2.85 |
| 720 | 640116 | 15, 16 (Normal Colon vs. Colon Tumor Tissue) | 19 | 4 | 5.02 | |
| | | 15, 17 (Normal Colon Tissue vs. Colon Metastasis) | 19 | 0 | 20.39 | |
| 721 | 6546 | 01, 02 (Colon, High Met vs. Colon, Low Met) | 0 | 9 | | 9.76 |
| 722 | 730866 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 723 | 4829 | 01, 02 (Colon, High Met vs. Colon, Low Met) | 0 | 7 | | 7.59 |
| 724 | 546632 | 23, 24 (Normal Lung Tissue vs. Lung Tumor Tissue) | 3 | 34 | | 11.22 |
| | | 16, 17 (Colon Tumor Tissue vs. Colon Metastasis) | 15 | 0 | 15.23 | |
| 725 | 549934 | 15, 16 (Normal Colon vs. Colon Tumor Tissue) | 11 | 3 | 3.88 | |
| | | 16, 17 (Colon Tumor Tissue vs. Colon Metastasis) | 3 | 20 | | 6.56 |
| | | 21, 22 (Normal Prostate vs. Prostate Cancer) | 8 | 0 | 7.87 | |
| 726 | 649655 | 16, 17 (Colon Tumor Tissue vs. Colon Metastasis) | 12 | 0 | 12.19 | |
| | | 15, 17 (Normal Colon Tissue vs. Colon Metastasis) | 6 | 0 | 6.44 | |
| 727 | 62016 | 16, 17 (Colon Tumor Tissue vs. Colon Metastasis) | 0 | 6 | | 5.91 |
| 728 | 2783 | 01, 02 (Colon, High Met vs. Colon, Low Met) | 27 | 6 | 4.15 | |
| | | 03, 04 (Breast, High Met vs. Breast, Non-Met) | 5 | 40 | | 8.2 |
| | | 15, 16 (Normal Colon vs. Colon Tumor Tissue) | 6 | 0 | 6.34 | |
| 729 | 3876 | 21, 22 (Normal Prostate vs. Prostate Cancer) | 9 | 26 | | 2.94 |
| | | 15, 17 (Normal Colon Tissue vs. Colon Metastasis) | 3 | 14 | | 4.35 |
| 730 | 20036 | 15, 17 (Normal Colon Tissue vs. Colon Metastasis) | 1 | 13 | | 12.11 |
| 731 | 644032 | 16, 17 (Colon Tumor Tissue vs. Colon Metastasis) | 124 | 0 | 125.92 | |
| | | 15, 16 (Normal Colon vs. Colon Tumor Tissue) | 7 | 124 | | 16.76 |
| | | 15, 17 (Normal Colon Tissue vs. Colon Metastasis) | 7 | 0 | 7.51 | |
| 732 | 451636 | 16, 17 (Colon Tumor Tissue vs. Colon Metastasis) | 1 | 8 | | 7.88 |
| 733 | 3428 | 03, 04 (Breast, High Met vs. Breast, Non-Met) | 20 | 0 | 19.51 | |
| 734 | 643954 | 16, 17 (Colon Tumor Tissue vs. Colon Metastasis) | 6 | 0 | 6.09 | |
| 735 | 456506 | 15, 17 (Normal Colon Tissue vs. Colon Metastasis) | 17 | 7 | 2.61 | |
| 736 | 449269 | 15, 17 (Normal Colon Tissue vs. Colon Metastasis) | 26 | 7 | 3.99 | |
| | | 15, 16 (Normal Colon vs. Colon Tumor Tissue) | 26 | 5 | 5.5 | |
| 737 | 732712 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 738 | 696 | 01, 02 (Colon, High Met vs. Colon, Low Met) | 24 | 87 | | 3.93 |
| | | 03, 04 (Breast, High Met vs. Breast, Non-Met) | 37 | 13 | 2.78 | |
| 739 | 456528 | 15, 16 (Normal Colon vs. Colon Tumor Tissue) | 0 | 7 | | 6.62 |
| 740 | 4043 | 01, 02 (Colon, High Met vs. Colon, Low Met) | 1 | 9 | | 9.76 |
| 741 | 3639 | 03, 04 (Breast, High Met vs. Breast, Non-Met) | 12 | 3 | 3.9 | |
| 742 | 1024 | 03, 04 (Breast, High Met vs. Breast, Non-Met) | 0 | 292 | | 299.28 |
| | | 08, 09 (Lung, High Met vs. Lung, Low Met) | 41 | 11 | 5.21 | |
| 743 | 1247 | 03, 04 (Breast, High Met vs. Breast, Non-Met) | 51 | 15 | 3.32 | |
| 744 | 4934 | 01, 02 (Colon, High Met vs. Colon, Low Met) | 1 | 9 | | 9.76 |
| 745 | 901 | 03, 04 (Breast, High Met vs. Breast, Non-Met) | 56 | 5 | 10.93 | |
| 746 | 452726 | 16, 17 (Colon Tumor Tissue vs. Colon Metastasis) | 8 | 1 | 8.12 | |
| 747 | 725825 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 748 | 456808 | 15, 16 (Normal Colon vs. Colon Tumor Tissue) | 7 | 42 | | 5.68 |
| | | 16, 17 (Colon Tumor Tissue vs. Colon Metastasis) | 42 | 1 | 42.65 | |
| 749 | 729295 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 750 | 551907 | 15, 16 (Normal Colon vs. Colon Tumor Tissue) | 7 | 0 | 7.4 | |
| 751 | 551527 | 15, 17 (Normal Colon Tissue vs. Colon Metastasis) | 0 | 7 | | 6.52 |
| 752 | 7098 | 03, 04 (Breast, High Met vs. Breast, Non-Met) | 10 | 0 | 9.76 | |
| 753 | 4589 | 01, 02 (Colon, High Met vs. Colon, Low Met) | 14 | 2 | 6.46 | |
| 754 | 554812 | 15, 17 (Normal Colon Tissue vs. Colon Metastasis) | 0 | 8 | | 7.45 |
| | | 16, 17 (Colon Tumor Tissue vs. Colon Metastasis) | 1 | 8 | | 7.88 |
| 755 | 3114 | 03, 04 (Breast, High Met vs. Breast, Non-Met) | 9 | 1 | 8.78 | |
| 756 | 6031 | 01, 02 (Colon, High Met vs. Colon, Low Met) | 9 | 1 | 8.3 | |
| 757 | 185628 | 03, 04 (Breast, High Met vs. Breast, Non-Met) | 0 | 6 | | 6.15 |
| 758 | 24719 | 03, 04 (Breast, High Met vs. Breast, Non-Met) | 4 | 26 | | 6.66 |
| | | 21, 22 (Normal Prostate vs. Prostate Cancer) | 4 | 14 | | 3.56 |
| 759 | 3428 | 03, 04 (Breast, High Met vs. Breast, Non-Met) | 20 | 0 | 19.51 | |
| 760 | 2676 | 01, 02 (Colon, High Met vs. Colon, Low Met) | 2 | 17 | | 9.22 |
| 761 | 649148 | 15, 17 (Normal Colon Tissue vs. Colon Metastasis) | 8 | 0 | 8.59 | |
| 762 | 234605 | 23, 24 (Normal Lung Tissue vs. Lung Tumor Tissue) | 17 | 5 | 3.43 | |
| | | 16, 17 (Colon Tumor Tissue vs. Colon Metastasis) | 39 | 14 | 2.83 | |
| | | 15, 17 (Normal Colon Tissue vs. Colon Metastasis) | 32 | 14 | 2.45 | |
| 763 | 2224 | 03, 04 (Breast, High Met vs. Breast, Non-Met) | 44 | 8 | 5.37 | |
| 764 | 185642 | 03, 04 (Breast, High Met vs. Breast, Non-Met) | 0 | 7 | | 7.17 |
| 765 | 649655 | 15, 17 (Normal Colon Tissue vs. Colon Metastasis) | 6 | 0 | 6.44 | |
| | | 16, 17 (Colon Tumor Tissue vs. Colon Metastasis) | 12 | 0 | 12.19 | |
| 766 | 2854 | 03, 04 (Breast, High Met vs. Breast, Non-Met) | 31 | 3 | 10.08 | |
| 767 | 453470 | 15, 16 (Normal Colon vs. Colon Tumor Tissue) | 12 | 1 | 12.68 | |
| | | 15, 17 (Normal Colon Tissue vs. Colon Metastasis) | 12 | 1 | 12.88 | |
| 768 | 11012 | 01, 02 (Colon, High Met vs. Colon, Low Met) | 0 | 6 | | 6.5 |
| 769 | 535208 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 770 | 448606 | 15, 17 (Normal Colon Tissue vs. Colon Metastasis) | 3 | 20 | | 6.21 |
| 771 | 12304 | 03, 04 (Breast, High Met vs. Breast, Non-Met) | 5 | 17 | | 3.48 |
| 772 | 2756 | 01, 02 (Colon, High Met vs. Colon, Low Met) | 28 | 8 | 3.23 | |
| 773 | 367 | 08, 09 (Lung, High Met vs. Lung, Low Met) | 30 | 99 | | 2.36 |
| | | 03, 04 (Breast, High Met vs. Breast, Non-Met) | 105 | 24 | 4.27 | |
| | | 01, 02 (Colon, High Met vs. Colon, Low Met) | 19 | 97 | | 5.53 |
| | | 15, 16 (Normal Colon vs. Colon Tumor Tissue) | 13 | 4 | 3.44 | |
| 774 | 11351 | 01, 02 (Colon, High Met vs. Colon, Low Met) | 0 | 6 | | 6.5 |
| 775 | 6858 | 01, 02 (Colon, High Met vs. Colon, Low Met) | 0 | 8 | | 8.67 |
| 776 | 7750 | 03, 04 (Breast, High Met vs. Breast, Non-Met) | 1 | 14 | | 14.35 |
| 777 | 6923 | 03, 04 (Breast, High Met vs. Breast, Non-Met) | 6 | 0 | 5.85 | |
| 778 | 11552 | 03, 04 (Breast, High Met vs. Breast, Non-Met) | 6 | 0 | 5.85 | |
| 779 | 12448 | 03, 04 (Breast, High Met vs. Breast, Non-Met) | 0 | 7 | | 7.17 |
| 781 | 10342 | 03, 04 (Breast, High Met vs. Breast, Non-Met) | 7 | 0 | 6.83 | |
| 782 | 9026 | 01, 02 (Colon, High Met vs. Colon, Low Met) | 7 | 0 | 6.46 | |
| 783 | 10342 | 03, 04 (Breast, High Met vs. Breast, Non-Met) | 7 | 0 | 6.83 | |
| 784 | 6455 | 03, 04 (Breast, High Met vs. Breast, Non-Met) | 6 | 0 | 5.85 | |
| 785 | 6455 | 03, 04 (Breast, High Met vs. Breast, Non-Met) | 6 | 0 | 5.85 | |
| 786 | 3416 | 01, 02 (Colon, High Met vs. Colon, Low Met) | 1 | 11 | | 11.93 |
| 787 | 3416 | 01, 02 (Colon, High Met vs. Colon, Low Met) | 1 | 11 | | 11.93 |
| 788 | 2889 | 01, 02 (Colon, High Met vs. Colon, Low Met) | 8 | 19 | | 2.57 |
| 789 | 7393 | 03, 04 (Breast, High Met vs. Breast, Non-Met) | 0 | 8 | | 8.2 |
| 790 | 14390 | 03, 04 (Breast, High Met vs. Breast, Non-Met) | 2 | 43 | | 22.04 |
| 791 | 661 | 03, 04 (Breast, High Met vs. Breast, Non-Met) | 77 | 10 | 7.51 | |
| | | 08, 09 (Lung, High Met vs. Lung, Low Met) | 0 | 10 | | 7.16 |
| 792 | 452992 | 15, 17 (Normal Colon Tissue vs. Colon Metastasis) | 7 | 0 | 7.51 | |
| 793 | 1943 | 03, 04 (Breast, High Met vs. Breast, Non-Met) | 16 | 4 | 3.9 | |
| | | 01, 02 (Colon, High Met vs. Colon, Low Met) | 9 | 29 | | 3.49 |
| | | 15, 16 (Normal Colon vs. Colon Tumor Tissue) | 6 | 19 | | 3 |
| 794 | 2027 | 03, 04 (Breast, High Met vs. Breast, Non-Met) | 12 | 35 | | 2.99 |
| 795 | 5482 | 01, 02 (Colon, High Met vs. Colon, Low Met) | 1 | 8 | | 8.67 |
| 796 | 650493 | 15, 17 (Normal Colon Tissue vs. Colon Metastasis) | 7 | 0 | 7.51 | |
| 797 | 640318 | 15, 17 (Normal Colon Tissue vs. Colon Metastasis) | 6 | 0 | 6.44 | |
| 798 | 646309 | 16, 17 (Colon Tumor Tissue vs. Colon Metastasis) | 6 | 0 | 6.09 | |
| 799 | 4316 | 03, 04 (Breast, High Met vs. Breast, Non-Met) | 0 | 6 | | 6.15 |
| 800 | 449701 | 16, 17 (Colon Tumor Tissue vs. Colon Metastasis) | 17 | 1 | 17.26 | |
| | | 15, 16 (Normal Colon vs. Colon Tumor Tissue) | 1 | 17 | | 16.08 |
| 801 | 560367 | 15, 16 (Normal Colon vs. Colon Tumor Tissue) | 0 | 9 | | 8.51 |
| 802 | 9997 | 03, 04 (Breast, High Met vs. Breast, Non-Met) | 7 | 0 | 6.83 | |
| 803 | 649106 | 15, 17 (Normal Colon Tissue vs. Colon Metastasis) | 7 | 0 | 7.51 | |
| 804 | 461835 | 15, 16 (Normal Colon vs. Colon Tumor Tissue) | 7 | 0 | 7.4 | |
| 805 | 640590 | 15, 17 (Normal Colon Tissue vs. Colon Metastasis) | 9 | 0 | 9.66 | |
| | | 15, 16 (Normal Colon vs. Colon Tumor Tissue) | 9 | 1 | 9.51 | |
| 806 | 648340 | 15, 17 (Normal Colon Tissue vs. Colon Metastasis) | 6 | 0 | 6.44 | |
| 807 | 554812 | 15, 17 (Normal Colon Tissue vs. Colon Metastasis) | 0 | 8 | | 7.45 |
| | | 16, 17 (Colon Tumor Tissue vs. Colon Metastasis) | 1 | 8 | | 7.88 |
| 808 | 447035 | 16, 17 (Colon Tumor Tissue vs. Colon Metastasis) | 8 | 1 | 8.12 | |
| 809 | 1208 | 03, 04 (Breast, High Met vs. Breast, Non-Met) | 13 | 0 | 12.68 | |
| 810 | 3114 | 03, 04 (Breast, High Met vs. Breast, Non-Met) | 9 | 1 | 8.78 | |
| 811 | 3114 | 03, 04 (Breast, High Met vs. Breast, Non-Met) | 9 | 1 | 8.78 | |
| 812 | 734078 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 813 | 450323 | 15, 17 (Normal Colon Tissue vs. Colon Metastasis) | 0 | 8 | | 7.45 |
| 814 | 11567 | 03, 04 (Breast, High Met vs. Breast, Non-Met) | 7 | 0 | 6.83 | |
| 815 | 11567 | 03, 04 (Breast, High Met vs. Breast, Non-Met) | 7 | 0 | 6.83 | |
| 816 | 6660 | 01, 02 (Colon, High Met vs. Colon, Low Met) | 7 | 0 | 6.46 | |
| 817 | 9026 | 01, 02 (Colon, High Met vs. Colon, Low Met) | 7 | 0 | 6.46 | |
| 818 | 185539 | 03, 04 (Breast, High Met vs. Breast, Non-Met) | 0 | 10 | | 10.25 |
| 819 | 3224 | 01, 02 (Colon, High Met vs. Colon, Low Met) | 17 | 2 | 7.84 | |
| 820 | 95700 | 03, 04 (Breast, High Met vs. Breast, Non-Met) | 1 | 21 | | 21.52 |
| 821 | 4439 | 08, 09 (Lung, High Met vs. Lung, Low Met) | 10 | 2 | 6.99 | |
| | | 01, 02 (Colon, High Met vs. Colon, Low Met) | 15 | 2 | 6.92 | |
| 822 | 3428 | 03, 04 (Breast, High Met vs. Breast, Non-Met) | 20 | 0 | 19.51 | |
| 823 | 1456 | 01, 02 (Colon, High Met vs. Colon, Low Met) | 9 | 22 | | 2.65 |
| | | 03, 04 (Breast, High Met vs. Breast, Non-Met) | 50 | 9 | 5.42 | |
| 824 | 11343 | 01, 02 (Colon, High Met vs. Colon, Low Met) | 0 | 6 | | 6.5 |
| 825 | 729206 | 15, 16 (Normal Colon vs. Colon Tumor Tissue) | 0 | 10 | | 9.46 |
| | | 16, 17 (Colon Tumor Tissue vs. Colon Metastasis) | 10 | 0 | 10.16 | |
| 826 | 558371 | 15, 16 (Normal Colon vs. Colon Tumor Tissue) | 10 | 2 | 5.28 | |
| | | 15, 17 (Normal Colon Tissue vs. Colon Metastasis) | 10 | 2 | 5.37 | |
| 827 | 451589 | 15, 17 (Normal Colon Tissue vs. Colon Metastasis) | 0 | 7 | | 6.52 |
| 828 | 404475 | 16, 17 (Colon Tumor Tissue vs. Colon Metastasis) | 11 | 2 | 5.59 | |
| | | 15, 17 (Normal Colon Tissue vs. Colon Metastasis) | 19 | 2 | 10.2 | |
| 829 | 734582 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 830 | 729779 | 15, 16 (Normal Colon vs. Colon Tumor Tissue) | 0 | 8 | | 7.57 |
| | | 16, 17 (Colon Tumor Tissue vs. Colon Metastasis) | 8 | 0 | 8.12 | |
| 831 | 555244 | 16, 17 (Colon Tumor Tissue vs. Colon Metastasis) | 0 | 11 | | 10.83 |
| 832 | 449269 | 15, 17 (Normal Colon Tissue vs. Colon Metastasis) | 26 | 7 | 3.99 | |
| | | 15, 16 (Normal Colon vs. Colon Tumor Tissue) | 26 | 5 | 5.5 | |
| 833 | 4609 | 01, 02 (Colon, High Met vs. Colon, Low Met) | 2 | 12 | | 6.5 |
| 834 | 640318 | 15, 17 (Normal Colon Tissue vs. Colon Metastasis) | 6 | 0 | 6.44 | |
| 835 | 729851 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 836 | 11028 | 01, 02 (Colon, High Met vs. Colon, Low Met) | 0 | 6 | | 6.5 |
| 837 | 643924 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 838 | 630259 | 15, 16 (Normal) Colon vs. Colon Tumor Tissue) | 7 | 0 | 7.4 | |
| 839 | 11286 | 01, 02 (Colon, High Met vs. Colon, Low Met) | 0 | 6 | | 6.5 |
| 840 | 185651 | 03, 04 (Breast, High Met vs. Breast, Non-Met) | 0 | 7 | | 7.17 |
| 841 | 7379 | 01, 02 (Colon, High Met vs. Colon, Low Met) | 0 | 6 | | 6.5 |
| 842 | 728408 | 15, 16 (Normal Colon vs. Colon Tumor Tissue) | 0 | 12 | | 11.35 |
| | | 16, 17 (Colon Tumor Tissue vs. Colon Metastasis) | 12 | 0 | 12.19 | |
| 843 | 646309 | 16, 17 (Colon Tumor Tissue vs. Colon Metastasis) | 6 | 0 | 6.09 | |
| 844 | 405073 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 845 | 185489 | 03, 04 (Breast, High Met vs. Breast, Non-Met) | 0 | 12 | | 12.3 |
| 846 | 447326 | 15, 17 (Normal Colon Tissue vs. Colon Metastasis) | 6 | 0 | 6.44 | |
| 847 | 11006 | 01, 02 (Colon, High Met vs. Colon, Low Met) | 0 | 6 | | 6.5 |
| 848 | 6863 | 01, 02 (Colon, High Met vs. Colon, Low Met) | 1 | 8 | | 8.67 |
| 849 | 11351 | 01, 02 (Colon, High Met vs. Colon, Low Met) | 0 | 6 | | 6.5 |
| 850 | 401553 | 15, 17 (Normal Colon Tissue vs. Colon Metastasis) | 8 | 0 | 8.59 | |
| 851 | 504513 | 15, 17 (Normal Colon Tissue vs. Colon Metastasis) | 6 | 0 | 6.44 | |
| 852 | 645979 | 16, 17 (Colon Tumor Tissue vs. Colon Metastasis) | 7 | 0 | 7.11 | |
| 853 | 6923 | 03, 04 (Breast, High Met vs. Breast, Non-Met) | 6 | 0 | 5.85 | |
| 854 | 1924 | 01, 02 (Colon, High Met vs. Colon, Low Met) | 8 | 21 | | 2.85 |
| 855 | 5838 | 01, 02 (Colon, High Met vs. Colon, Low Met) | 1 | 9 | | 9.76 |
| 856 | 2062 | 13, 14 (bFGF Treated HMVEC vs. VEGF-Treated HMVEC) (bFGF | 0 | 7 | | 6.88 |
| | | Treated HMVEC vs. VEGF-Treated HMVEC) | | | | |
| | | 03, 04 (Breast, High Met vs. Breast, Non-Met) | 8 | 19 | | 2.43 |
| | | 01, 02 (Colon, High Met vs. Colon, Low Met) | 9 | 39 | | 4.7 |
| 857 | 447388 | 15, 17 (Normal Colon Tissue vs. Colon Metastasis) | 142 | 4 | 38.1 | |
| | | 18, 19 (Normal Colon Tissue vs. Colon Tumor) | 18 | 0 | 20.57 | |
| | | 18, 20 (Normal Colon Tissue vs. Colon Metastasis) | 18 | 0 | 15.39 | |
| | | 15, 16 (Normal Colon vs. Colon Tumor Tissue) | 142 | 2 | 75.05 | |
| 858 | 12419 | 03, 04 (Breast, High Met vs. Breast, Non-Met) | 0 | 15 | | 15.37 |
| 859 | 3224 | 01, 02 (Colon, High Met vs. Colon, Low Met) | 17 | 2 | 7.84 | |
| 860 | 5474 | 03, 04 (Breast, High Met vs. Breast, Non-Met) | 5 | 17 | | 3.48 |
| | | 01, 02 (Colon, High Met vs. Colon, Low Met) | 1 | 8 | | 8.67 |
| 861 | 3522 | 01, 02 (Colon, High Met vs. Colon, Low Met) | 3 | 12 | | 4.34 |
| 862 | 731785 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 863 | 3765 | 01, 02 (Colon, High Met vs. Colon, Low Met) | 19 | 6 | 2.92 | |
| 864 | 640323 | 16, 17 (Colon Tumor Tissue vs. Colon Metastasis) | 6 | 0 | 6.09 | |
| | | 15, 17 (Normal Colon Tissue vs. Colon Metastasis) | 8 | 0 | 8.59 | |
| 865 | 379105 | 15, 16 (Normal Colon vs. Colon Tumor Tissue) | 0 | 8 | | 7.57 |
| 866 | 448029 | 16, 17 (Colon Tumor Tissue vs. Colon Metastasis) | 10 | 1 | 10.16 | |
| 867 | 650476 | 15, 17 (Normal Colon Tissue vs. Colon Metastasis) | 7 | 0 | 7.51 | |
| 868 | 640525 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 869 | 390124 | 15, 17 (Normal Colon Tissue vs. Colon Metastasis) | 6 | 18 | | 2.79 |
| | | 19, 20 (Colon Tumor Tissue vs. Colon Metastasis) | 8 | 0 | 5.98 | |
| | | 18, 19 (Normal Colon Tissue vs. Colon Tumor) | 0 | 8 | | 7 |
| 870 | 464029 | 16, 17 (Colon Tumor Tissue vs. Colon Metastasis) | 1 | 9 | | 8.86 |
| 871 | 468109 | 15, 17 (Normal Colon Tissue vs. Colon Metastasis) | 6 | 0 | 6.44 | |
| 872 | 21669 | 16, 17 (Colon Tumor Tissue vs. Colon Metastasis) | 0 | 7 | | 6.89 |
| 873 | 651088 | 15, 17 (Normal Colon Tissue vs. Colon Metastasis) | 6 | 0 | 6.44 | |
| 874 | 2737 | 16, 17 (Colon Tumor Tissue vs. Colon Metastasis) | 3 | 14 | | 4.6 |
| | | 15, 17 (Normal Colon Tissue vs. Colon Metastasis) | 4 | 14 | | 3.26 |
| 875 | 556421 | 15, 16 (Normal Colon vs. Colon Tumor Tissue) | 12 | 1 | 12.68 | |
| 876 | 452245 | 16, 17 (Colon Tumor Tissue vs. Colon Metastasis) | 1 | 12 | | 11.82 |
| | | 15, 17 (Normal Colon Tissue vs. Colon Metastasis) | 1 | 12 | | 11.18 |
| 877 | 447539 | 15, 17 (Normal Colon Tissue vs. Colon Metastasis) | 8 | 0 | 8.59 | |
| | | 15, 16 (Normal Colon vs. Colon Tumor Tissue) | 8 | 1 | 8.46 | |
| 878 | 546642 | 16, 17 (Colon Tumor Tissue vs. Colon Metastasis) | 11 | 0 | 11.17 | |
| 879 | 236368 | 15, 16 (Normal Colon vs. Colon Tumor Tissue) | 271 | 16 | 17.9 | |
| | | 15, 17 (Normal Colon Tissue vs. Colon Metastasis) | 271 | 0 | 290.88 | |
| | | 16, 17 (Colon Tumor Tissue vs. Colon Metastasis) | 16 | 0 | 16.25 | |
| | | 18, 19 (Normal Colon Tissue vs. Colon Tumor) | 9 | 1 | 10.29 | |
| | | 18, 20 (Normal Colon Tissue vs. Colon Metastasis) | 9 | 0 | 7.69 | |
| 880 | 644523 | 16, 17 (Colon Tumor Tissue vs. Colon Metastasis) | 6 | 0 | 6.09 | |
| 881 | 729173 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 882 | 8315 | 03, 04 (Breast, High Met vs. Breast, Non-Met) | 6 | 0 | 5.85 | |
| 883 | 450463 | 15, 16 (Normal Colon vs. Colon Tumor Tissue) | 31 | 13 | 2.52 | |
| 884 | 650856 | 16, 17 (Colon Tumor Tissue vs. Colon Metastasis) | 6 | 0 | 6.09 | |
| 885 | 648109 | 15, 16 (Normal Colon vs. Colon Tumor Tissue) | 8 | 1 | 8.46 | |
| | | 15, 17 (Normal Colon Tissue vs. Colon Metastasis) | 8 | 0 | 8.59 | |
| 886 | 726644 | 16, 17 (Colon Tumor Tissue vs. Colon Metastasis) | 18 | 0 | 18.28 | |
| | | 15, 16 (Normal Colon vs. Colon Tumor Tissue) | 0 | 18 | | 17.03 |
| 887 | 727224 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 888 | 557906 | 15, 16 (Normal Colon vs. Colon Tumor Tissue) | 12 | 3 | 4.23 | |
| 889 | 502683 | 15, 16 (Normal Colon vs. Colon Tumor Tissue) | 6 | 0 | 6.34 | |
| 890 | 728408 | 15, 16 (Normal Colon vs. Colon Tumor Tissue) | 0 | 12 | | 11.35 |
| | | 16, 17 (Colon Tumor Tissue vs. Colon Metastasis) | 12 | 0 | 12.19 | |
| 891 | 647952 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 892 | 639991 | 15, 17 (Normal Colon Tissue vs. Colon Metastasis) | 6 | 0 | 6.44 | |
| 893 | 735346 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 894 | 102655 | 15, 16 (Normal Colon vs. Colon Tumor Tissue) | 14 | 0 | 14.8 | |
| | | 15, 17 (Normal Colon Tissue vs. Colon Metastasis) | 14 | 0 | 15.03 | |
| | | 03, 04 (Breast, High Met vs. Breast, Non-Met) | 4 | 33 | | 8.46 |
| 895 | 553629 | 16, 17 (Colon Tumor Tissue vs. Colon Metastasis) | 4 | 17 | | 4.19 |
| | | 15, 17 (Normal Colon Tissue vs. Colon Metastasis) | 4 | 17 | | 3.96 |
| 896 | 1609 | 01, 02 (Colon, High Met vs. Colon, Low Met) | 3 | 58 | | 20.96 |
| | | 03, 04 (Breast, High Met vs. Breast, Non-Met) | 10 | 2 | 4.88 | |
| | | 15, 17 (Normal Colon Tissue vs. Colon Metastasis) | 15 | 3 | 5.37 | |
| | | 16, 17 (Colon Tumor Tissue vs. Colon Metastasis) | 14 | 3 | 4.74 | |
| 897 | 641884 | 15, 17 (Normal Colon Tissue vs. Colon Metastasis) | 8 | 0 | 8.59 | |
| | | 16, 17 (Colon Tumor Tissue vs. Colon Metastasis) | 7 | 0 | 7.11 | |
| 898 | 648872 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 899 | 644242 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 900 | 63559 | 03, 04 (Breast, High Met vs. Breast, Non-Met) | 2 | 12 | | 6.15 |
| 901 | 550108 | 15, 17 (Normal Colon Tissue vs. Colon Metastasis) | 6 | 37 | | 5.75 |
| | | 15, 16 (Normal Colon vs. Colon Tumor Tissue) | 6 | 19 | | 3 |
| 902 | 374306 | 15, 17 (Normal Colon Tissue vs. Colon Metastasis) | 1 | 10 | | 9.32 |
| 903 | 5838 | 01, 02 (Colon, High Met vs. Colon, Low Met) | 1 | 9 | | 9.76 |
| 904 | 645530 | 15, 17 (Normal Colon Tissue vs. Colon Metastasis) | 10 | 0 | 10.73 | |
| | | 15, 16 (Normal Colon vs. Colon Tumor Tissue) | 10 | 2 | 5.28 | |
| 905 | 649732 | 16, 17 (Colon Tumor Tissue vs. Colon Metastasis) | 7 | 0 | 7.11 | |
| 906 | 649143 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 907 | 7571 | 01, 02 (Colon, High Met vs. Colon, Low Met) | 0 | 8 | | 8.67 |
| 908 | 4572 | 03, 04 (Breast, High Met vs. Breast, Non-Met) | 2 | 11 | | 5.64 |
| 909 | 2147 | 01, 02 (Colon, High Met vs. Colon, Low Met) | 31 | 6 | 4.77 | |
| | | 03, 04 (Breast, High Met vs. Breast, Non-Met) | 12 | 2 | 5.85 | |
| 910 | 462659 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 911 | 727723 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 912 | 2636 | 08, 09 (Lung, High Met vs. Lung, Low Met) | 7 | 1 | 9.78 | |
| | | 01, 02 (Colon, High Met vs. Colon, Low Met) | 8 | 19 | | 2.57 |
| 913 | 500959 | 15, 17 (Normal Colon Tissue vs. Colon Metastasis) | 8 | 0 | 8.59 | |
| | | 15, 16 (Normal Colon vs. Colon Tumor Tissue) | 8 | 0 | 8.46 | |
| 914 | 3428 | 03, 04 (Breast, High Met vs. Breast, Non-Met) | 20 | 0 | 19.51 | |
| 915 | 734929 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 916 | 453592 | 15, 16 (Normal Colon vs. Colon Tumor Tissue) | 19 | 6 | 3.35 | |
| 917 | 15414 | 15, 17 (Normal Colon Tissue vs. Colon Metastasis) | 1 | 9 | | 8.38 |
| 918 | 648959 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 919 | 453470 | 15, 17 (Normal Colon Tissue vs. Colon Metastasis) | 12 | 1 | 12.88 | |
| | | 15, 16 (Normal Colon vs. Colon Tumor Tissue) | 12 | 1 | 12.68 | |
| 920 | 649272 | 16, 17 (Colon Tumor Tissue vs. Colon Metastasis) | 8 | 0 | 8.12 | |
| 921 | 1699 | 15, 16 (Normal Colon vs. Colon Tumor Tissue) | 1 | 10 | | 9.46 |
| | | 03, 04 (Breast, High Met vs. Breast, Non-Met) | 37 | 12 | 3.01 | |
| 922 | 649719 | 15, 17 (Normal Colon Tissue vs. Colon Metastasis) | 6 | 0 | 6.44 | |
| 923 | 562805 | 16, 17 (Colon Tumor Tissue vs. Colon Metastasis) | 0 | 6 | | 5.91 |
| 924 | 452204 | 16, 17 (Colon Tumor Tissue vs. Colon Metastasis) | 1 | 8 | | 7.88 |
| | | 23, 24 (Normal Lung Tissue vs. Lung Tumor Tissue) | 3 | 13 | | 4.29 |
| | | 15, 16 (Normal Colon vs. Colon Tumor Tissue) | 9 | 1 | 9.51 | |
| 925 | 549178 | 16, 17 (Colon Tumor Tissue vs. Colon Metastasis) | 0 | 10 | | 9.85 |
| | | 15, 17 (Normal Colon Tissue vs. Colon Metastasis) | 1 | 10 | | 9.32 |
| 926 | 639177 | 15, 17 (Normal Colon Tissue vs. Colon Metastasis) | 6 | 0 | 6.44 | |
| 927 | 562550 | 16, 17 (Colon Tumor Tissue vs. Colon Metastasis) | 0 | 6 | | 5.91 |
| 928 | 561807 | 16, 17 (Colon Tumor Tissue vs. Colon Metastasis) | 1 | 8 | | 7.88 |
| 929 | 641373 | 15, 17 (Normal Colon Tissue vs. Colon Metastasis) | 15 | 0 | 16.1 | |
| | | 15, 16 (Normal Colon vs. Colon Tumor Tissue) | 15 | 3 | 5.28 | |
| 930 | 514418 | 16, 17 (Colon Tumor Tissue vs. Colon Metastasis) | 0 | 9 | | 8.86 |
| | | 15, 17 (Normal Colon Tissue vs. Colon Metastasis) | 1 | 9 | | 8.38 |
| 931 | 567078 | 16, 17 (Colon Tumor Tissue vs. Colon Metastasis) | 11 | 1 | 11.17 | |
| 932 | 643061 | 15, 17 (Normal Colon Tissue vs. Colon Metastasis) | 8 | 0 | 8.59 | |
| | | 15, 16 (Normal Colon vs. Colon Tumor Tissue) | 8 | 1 | 8.46 | |
| 933 | 549160 | 15, 17 (Normal Colon Tissue vs. Colon Metastasis) | 0 | 7 | | 6.52 |
| 934 | 449269 | 15, 17 (Normal Colon Tissue vs. Colon Metastasis) | 26 | 7 | 3.99 | |
| | | 15, 16 (Normal Colon vs. Colon Tumor Tissue) | 26 | 5 | 5.5 | |
| 935 | 453082 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 936 | 418135 | 16, 17 (Colon Tumor Tissue vs. Colon Metastasis) | 6 | 23 | | 3.77 |
| | | 15, 17 (Normal Colon Tissue vs. Colon Metastasis) | 1 | 23 | | 21.43 |
| 937 | 2783 | 01, 02 (Colon, High Met vs. Colon, Low Met) | 27 | 6 | 4.15 | |
| | | 03, 04 (Breast, High Met vs. Breast, Non-Met) | 5 | 40 | | 8.2 |
| | | 15, 16 (Normal Colon vs. Colon Tumor Tissue) | 6 | 0 | 6.34 | |
| 938 | 549435 | 15, 16 (Normal Colon vs. Colon Tumor Tissue) | 14 | 2 | 7.4 | |
| 939 | 446614 | 16, 17 (Colon Tumor Tissue vs. Colon Metastasis) | 7 | 0 | 7.11 | |
| 940 | 449477 | 15, 17 (Normal Colon Tissue vs. Colon Metastasis) | 2 | 12 | | 5.59 |
| 941 | 454380 | 15, 16 (Normal Colon vs. Colon Tumor Tissue) | 3 | 14 | | 4.42 |
| 942 | 450914 | 15, 16 (Normal Colon vs. Colon Tumor Tissue) | 0 | 6 | | 5.68 |
| 943 | 736860 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 944 | 727224 | 15, 16 (Normal Colon vs. Colon Tumor Tissue) | 0 | 8 | | 7.57 |
| | | 16, 17 (Colon Tumor Tissue vs. Colon Metastasis) | 8 | 0 | 8.12 | |
| 945 | 644242 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 946 | 562550 | 16, 17 (Colon Tumor Tissue vs. Colon Metastasis) | 0 | 6 | | 5.91 |
| 947 | 649148 | 15, 17 (Normal Colon Tissue vs. Colon Metastasis) | 8 | 0 | 8.59 | |
| 948 | 375889 | 15, 17 (Normal Colon Tissue vs. Colon Metastasis) | 0 | 7 | | 6.52 |
| 949 | 449437 | 15, 16 (Normal Colon vs. Colon Tumor Tissue) | 14 | 3 | 4.93 | |
| | | 16, 17 (Colon Tumor Tissue vs. Colon Metastasis) | 3 | 12 | | 3.94 |
| 950 | 449044 | 15, 17 (Normal Colon Tissue vs. Colon Metastasis) | 6 | 0 | 6.44 | |
| | | 16, 17 (Colon Tumor Tissue vs. Colon Metastasis) | 9 | 0 | 9.14 | |
| 951 | 555318 | 16, 17 (Colon Tumor Tissue vs. Colon Metastasis) | 2 | 12 | | 5.91 |
| 952 | 456764 | 16, 17 (Colon Tumor Tissue vs. Colon Metastasis) | 10 | 1 | 10.16 | |
| | | 15, 17 (Normal Colon Tissue vs. Colon Metastasis) | 14 | 1 | 15.03 | |
| 953 | 11567 | 03, 04 (Breast, High Met vs. Breast, Non-Met) | 7 | 0 | 6.83 | |
| 954 | 3522 | 01, 02 (Colon, High Met vs. Colon, Low Met) | 3 | 12 | | 4.34 |
| 955 | 456528 | 15, 16 (Normal Colon vs. Colon Tumor Tissue) | 0 | 7 | | 6.62 |
| 956 | 639142 | 15, 17 (Normal Colon Tissue vs. Colon Metastasis) | 8 | 0 | 8.59 | |
| 957 | 446371 | 15, 16 (Normal Colon vs. Colon Tumor Tissue) | 6 | 0 | 6.34 | |
| 958 | 554742 | 16, 17 (Colon Tumor Tissue vs. Colon Metastasis) | 0 | 6 | | 5.91 |
| 959 | 448029 | 16, 17 (Colon Tumor Tissue vs. Colon Metastasis) | 10 | 1 | 10.16 | |
| 960 | 551380 | 16, 17 (Colon Tumor Tissue vs. Colon Metastasis) | 4 | 14 | | 3.45 |
| 961 | 551527 | 15, 17 (Normal Colon Tissue vs. Colon Metastasis) | 0 | 7 | | 6.52 |
| 962 | 729295 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 963 | 349744 | 16, 17 (Colon Tumor Tissue vs. Colon Metastasis) | 0 | 6 | | 5.91 |
| 964 | 648996 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 965 | 447126 | 15, 16 (Normal Colon vs. Colon Tumor Tissue) | 19 | 5 | 4.02 | |
| | | 16, 17 (Colon Tumor Tissue vs. Colon Metastasis) | 5 | 20 | | 3.94 |
| 966 | 730866 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 967 | 420686 | 16, 17 (Colon Tumor Tissue vs. Colon Metastasis) | 0 | 8 | | 7.88 |
| 968 | 451753 | 23, 24 (Normal Lung Tissue vs. Lung Tumor Tissue) | 1 | 12 | | 11.88 |
| | | 15, 16 (Normal Colon vs. Colon Tumor Tissue) | 4 | 15 | | 3.55 |
| 969 | 451380 | 15, 16 (Normal Colon vs. Colon Tumor Tissue) | 20 | 7 | 3.02 | |
| 970 | 645530 | 15, 16 (Normal Colon vs. Colon Tumor Tissue) | 10 | 2 | 5.28 | |
| | | 15, 17 (Normal Colon Tissue vs. Colon Metastasis) | 10 | 0 | 10.73 | |
| 971 | 554703 | 16, 17 (Colon Tumor Tissue vs. Colon Metastasis) | 1 | 9 | | 8.86 |
| | | 15, 17 (Normal Colon Tissue vs. Colon Metastasis) | 0 | 9 | | 8.38 |
| 972 | 562835 | 15, 17 (Normal Colon Tissue vs. Colon Metastasis) | 0 | 9 | | 8.38 |
| 973 | 732764 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 974 | 556216 | 16, 17 (Colon Tumor Tissue vs. Colon Metastasis) | 0 | 7 | | 6.89 |
| | | 15, 17 (Normal Colon Tissue vs. Colon Metastasis) | 0 | 7 | | 6.52 |
| 975 | 728779 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 976 | 414739 | 16, 17 (Colon Tumor Tissue vs. Colon Metastasis) | 2 | 14 | | 6.89 |
| | | 15, 17 (Normal Colon Tissue vs. Colon Metastasis) | 2 | 14 | | 6.52 |
| 977 | 551514 | 16, 17 (Colon Tumor Tissue vs. Colon Metastasis) | 1 | 13 | | 12.8 |
| | | 15, 16 (Normal Colon vs. Colon Tumor Tissue) | 8 | 1 | 8.46 | |
| 978 | 550107 | 15, 16 (Normal Colon vs. Colon Tumor Tissue) | 36 | 14 | 2.72 | |
| 979 | 726786 | 15, 16 (Normal Colon vs. Colon Tumor Tissue) | 0 | 8 | | 7.57 |
| | | 16, 17 (Colon Tumor Tissue vs. Colon Metastasis) | 8 | 0 | 8.12 | |
| 980 | 456747 | 15, 17 (Normal Colon Tissue vs. Colon Metastasis) | 6 | 0 | 6.44 | |
| 981 | 562550 | 16, 17 (Colon Tumor Tissue vs. Colon Metastasis) | 0 | 6 | | 5.91 |
| 982 | 549722 | 16, 17 (Colon Tumor Tissue vs. Colon Metastasis) | 0 | 6 | | 5.91 |
| 983 | 640525 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 984 | 455542 | 15, 16 (Normal Colon vs. Colon Tumor Tissue) | 0 | 6 | | 5.68 |
| 985 | 9436 | 16, 17 (Colon Tumor Tissue vs. Colon Metastasis) | 4 | 15 | | 3.69 |
| 986 | 380284 | 15, 16 (Normal Colon vs. Colon Tumor Tissue) | 9 | 0 | 9.51 | |
| | | 15, 17 (Normal Colon Tissue vs. Colon Metastasis) | 9 | 1 | 9.66 | |
| 987 | 556260 | 16, 17 (Colon Tumor Tissue vs. Colon Metastasis) | 0 | 6 | | 5.91 |
| 988 | 650476 | 15, 17 (Normal Colon Tissue vs. Colon Metastasis) | 7 | 0 | 7.51 | |
| 989 | 554500 | 15, 16 (Normal Colon vs. Colon Tumor Tissue) | 6 | 0 | 6.34 | |
| 990 | 422375 | 15, 17 (Normal Colon Tissue vs. Colon Metastasis) | 2 | 11 | | 5.12 |
| 991 | 456528 | 15, 16 (Normal Colon vs. Colon Tumor Tissue) | 0 | 7 | | 6.62 |
| 992 | 644190 | 16, 17 (Colon Tumor Tissue vs. Colon Metastasis) | 6 | 0 | 6.09 | |
| 993 | 554080 | 16, 17 (Colon Tumor Tissue vs. Colon Metastasis) | 1 | 9 | | 8.86 |
| 994 | 546705 | 16, 17 (Colon Tumor Tissue vs. Colon Metastasis) | 0 | 6 | | 5.91 |
| 995 | 558337 | 16, 17 (Colon Tumor Tissue vs. Colon Metastasis) | 0 | 6 | | 5.91 |
| 996 | 449269 | 15, 17 (Normal Colon Tissue vs. Colon Metastasis) | 26 | 7 | 3.99 | |
| | | 15, 16 (Normal Colon vs. Colon Tumor Tissue) | 26 | 5 | 5.5 | |
| 997 | 645799 | 16, 17 (Colon Tumor Tissue vs. Colon Metastasis) | 7 | 0 | 7.11 | |
| 998 | 456506 | 15, 17 (Normal Colon Tissue vs. Colon Metastasis) | 17 | 7 | 2.61 | |
| 999 | 218416 | 16, 17 (Colon Tumor Tissue vs. Colon Metastasis) | 0 | 6 | | 5.91 |
| 1000 | 455820 | 16, 17 (Colon Tumor Tissue vs. Colon Metastasis) | 0 | 7 | | 6.89 |
| 1001 | 554703 | 16, 17 (Colon Tumor Tissue vs. Colon Metastasis) | 1 | 9 | | 8.86 |
| | | 15, 17 (Normal Colon Tissue vs. Colon Metastasis) | 0 | 9 | | 8.38 |
| 1002 | 650204 | 15, 17 (Normal Colon Tissue vs. Colon Metastasis) | 11 | 0 | 11.81 | |
| 1003 | 456808 | 15, 16 (Normal Colon vs. Colon Tumor Tissue) | 7 | 42 | | 5.68 |
| | | 16, 17 (Colon Tumor Tissue vs. Colon Metastasis) | 42 | 1 | 42.65 | |
| 1004 | 420686 | 16, 17 (Colon Tumor Tissue vs. Colon Metastasis) | 0 | 8 | | 7.88 |
| 1005 | 378373 | 16, 17 (Colon Tumor Tissue vs. Colon Metastasis) | 0 | 7 | | 6.89 |
| | | 15, 17 (Normal Colon Tissue vs. Colon Metastasis) | 0 | 7 | | 6.52 |
| 1006 | 463824 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1007 | 24939 | 15, 16 (Normal Colon vs. Colon Tumor Tissue) | 0 | 8 | | 7.57 |
| 1008 | 556561 | 16, 17 (Colon Tumor Tissue vs. Colon Metastasis) | 1 | 10 | | 9.85 |
| | | 15, 17 (Normal Colon Tissue vs. Colon Metastasis) | 0 | 10 | | 9.32 |
| 1009 | 380406 | 16, 17 (Colon Tumor Tissue vs. Colon Metastasis) | 3 | 12 | | 3.94 |
| | | 15, 16 (Normal Colon vs. Colon Tumor Tissue) | 16 | 3 | 5.64 | |
| 1010 | 456764 | 15, 17 (Normal Colon Tissue vs. Colon Metastasis) | 14 | 1 | 15.03 | |
| | | 16, 17 (Colon Tumor Tissue vs. Colon Metastasis) | 10 | 1 | 10.16 | |
| 1011 | 725703 | 15, 16 (Normal Colon vs. Colon Tumor Tissue) | 0 | 9 | | 8.51 |
| | | 16, 17 (Colon Tumor Tissue vs. Colon Metastasis) | 9 | 0 | 9.14 | |
| 1012 | 185465 | 03, 04 (Breast, High Met vs. Breast, Non-Met) | 2 | 14 | | 7.17 |
| | | 16, 17 (Colon Tumor Tissue vs. Colon Metastasis) | 4 | 14 | | 3.45 |
| | | 23, 24 (Normal Lung Tissue vs. Lung Tumor Tissue) | 25 | 9 | 2.81 | |
| 1013 | 5830 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1014 | 539955 | 16, 17 (Colon Tumor Tissue vs. Colon Metastasis) | 24 | 0 | 24.37 | |
| | | 15, 17 (Normal Colon Tissue vs. Colon Metastasis) | 11 | 0 | 11.81 | |
| | | 23, 24 (Normal Lung Tissue vs. Lung Tumor Tissue) | 3 | 47 | | 15.51 |
| 1015 | 640747 | 15, 17 (Normal Colon Tissue vs. Colon Metastasis) | 6 | 0 | 6.44 | |
| 1016 | 500630 | 16, 17 (Colon Tumor Tissue vs. Colon Metastasis) | 6 | 0 | 6.09 | |
| 1017 | 448511 | 16, 17 (Colon Tumor Tissue vs. Colon Metastasis) | 4 | 26 | | 6.4 |
| | | 15, 17 (Normal Colon Tissue vs. Colon Metastasis) | 3 | 26 | | 8.07 |
| 1018 | 405073 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1019 | 641439 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1020 | 406092 | 15, 17 (Normal Colon Tissue vs. Colon Metastasis) | 12 | 3 | 4.29 | |
| 1021 | 559806 | 16, 17 (Colon Tumor Tissue vs. Colon Metastasis) | 0 | 6 | | 5.91 |
| 1022 | 380284 | 15, 16 (Normal Colon vs. Colon Tumor Tissue) | 9 | 0 | 9.51 | |
| | | 15, 17 (Normal Colon Tissue vs. Colon Metastasis) | 9 | 1 | 9.66 | |
| 1023 | 560700 | 16, 17 (Colon Tumor Tissue vs. Colon Metastasis) | 0 | 6 | | 5.91 |
| 1024 | 552879 | 16, 17 (Colon Tumor Tissue vs. Colon Metastasis) | 2 | 14 | | 6.89 |
| | | 15, 16 (Normal Colon vs. Colon Tumor Tissue) | 11 | 2 | 5.81 | |
| 1025 | 640590 | 15, 17 (Normal Colon Tissue vs. Colon Metastasis) | 9 | 0 | 9.66 | |
| | | 15, 16 (Normal Colon vs. Colon Tumor Tissue) | 9 | 1 | 9.51 | |
| 1026 | 641683 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 1027 | 648934 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 1028 | 557948 | 16, 17 (Colon Tumor Tissue vs. Colon Metastasis) | 0 | 11 | | 10.83 |
| 1029 | 377094 | 15, 16 (Normal Colon vs. Colon Tumor Tissue) | 10 | 2 | 5.28 | |
| 1030 | 449617 | 16, 17 (Colon Tumor Tissue vs. Colon Metastasis) | 7 | 18 | | 2.53 |
| 1031 | 978 | 15, 17 (Normal Colon Tissue vs. Colon Metastasis) | 67 | 20 | 3.6 | |
| | | 15, 16 (Normal Colon vs. Colon Tumor Tissue) | 67 | 28 | 2.53 | |
| | | 03, 04 (Breast, High Met vs. Breast, Non-Met) | 78 | 23 | 3.31 | |
| 1032 | 607430 | 15, 16 (Normal Colon vs. Colon Tumor Tissue) | 6 | 0 | 6.34 | |
| 1033 | 641837 | 15, 17 (Normal Colon Tissue vs. Colon Metastasis) | 16 | 0 | 17.17 | |
| | | 16, 17 (Colon Tumor Tissue vs. Colon Metastasis) | 31 | 0 | 31.48 | |
| 1034 | 449750 | 16, 17 (Colon Tumor Tissue vs. Colon Metastasis) | 1 | 28 | | 27.57 |
| | | 15, 17 (Normal Colon Tissue vs. Colon Metastasis) | 4 | 28 | | 6.52 |
| 1035 | 646780 | 15, 17 (Normal Colon Tissue vs. Colon Metastasis) | 10 | 0 | 10.73 | |
| | | 16, 17 (Colon Tumor Tissue vs. Colon Metastasis) | 7 | 0 | 7.11 | |
| 1036 | 546642 | 16, 17 (Colon Tumor Tissue vs. Colon Metastasis) | 11 | 0 | 11.17 | |
| 1037 | 642906 | 16, 17 (Colon Tumor Tissue vs. Colon Metastasis) | 7 | 0 | 7.11 | |
| 1038 | 552879 | 16, 17 (Colon Tumor Tissue vs. Colon Metastasis) | 2 | 14 | | 6.89 |
| | | 15, 16 (Normal Colon vs. Colon Tumor Tissue) | 11 | 2 | 5.81 | |
| 1039 | 644205 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1040 | 506744 | 16, 17 (Colon Tumor Tissue vs. Colon Metastasis) | 0 | 8 | | 7.88 |
| | | 15, 16 (Normal Colon vs. Colon Tumor Tissue) | 7 | 0 | 7.4 | |
| 1041 | 557797 | 16, 17 (Colon Tumor Tissue vs. Colon Metastasis) | 3 | 16 | | 5.25 |
| 1042 | 640356 | 15, 17 (Normal Colon Tissue vs. Colon Metastasis) | 10 | 0 | 10.73 | |
| 1043 | 462659 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 1044 | 645633 | 15, 17 (Normal Colon Tissue vs. Colon Metastasis) | 7 | 0 | 7.51 | |
| 1045 | 237288 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1046 | 454343 | 16, 17 (Colon Tumor Tissue vs. Colon Metastasis) | 10 | 0 | 10.16 | |
| 1047 | 386543 | 16, 17 (Colon Tumor Tissue vs. Colon Metastasis) | 3 | 23 | | 7.55 |
| | | 15, 17 (Normal Colon Tissue vs. Colon Metastasis) | 3 | 23 | | 7.14 |
| 1048 | 446404 | 18, 20 (Normal Colon Tissue vs. Colon Metastasis) | 23 | 0 | 19.66 | |
| | | 15, 17 (Normal Colon Tissue vs. Colon Metastasis) | 73 | 0 | 78.36 | |
| | | 18, 19 (Normal Colon Tissue vs. Colon Tumor) | 23 | 0 | 26.28 | |
| | | 15, 16 (Normal Colon vs. Colon Tumor Tissue) | 73 | 1 | 77.16 | |
| 1049 | 456528 | 15, 16 (Normal Colon vs. Colon Tumor Tissue) | 0 | 7 | | 6.62 |
| 1050 | 456528 | 15, 16 (Normal Colon vs. Colon Tumor Tissue) | 0 | 7 | | 6.62 |
| 1051 | 452781 | 15, 17 (Normal Colon Tissue vs. Colon Metastasis) | 2 | 11 | | 5.12 |
| 1052 | 551671 | 16, 17 (Colon Tumor Tissue vs. Colon Metastasis) | 0 | 7 | | 6.89 |
| | | 15, 17 (Normal Colon Tissue vs. Colon Metastasis) | 0 | 7 | | 6.52 |
| 1053 | 644242 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1054 | 561892 | 15, 17 (Normal Colon Tissue vs. Colon Metastasis) | 0 | 7 | | 6.52 |
| 1055 | 450429 | 16, 17 (Colon Tumor Tissue vs. Colon Metastasis) | 10 | 1 | 10.16 | |
| | | 15, 17 (Normal Colon Tissue vs. Colon Metastasis) | 13 | 1 | 13.95 | |
| 1056 | 533588 | 15, 17 (Normal Colon Tissue vs. Colon Metastasis) | 7 | 0 | 7.51 | |
| 1057 | 553877 | 16, 17 (Colon Tumor Tissue vs. Colon Metastasis) | 1 | 8 | | 7.88 |
| 1058 | 650195 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1059 | 193486 | 15, 17 (Normal Colon Tissue vs. Colon Metastasis) | 0 | 7 | | 6.52 |
| 1060 | 650195 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1061 | 562835 | 15, 17 (Normal Colon Tissue vs. Colon Metastasis) | 0 | 9 | | 8.38 |
| 1062 | 736816 | 16, 17 (Colon Tumor Tissue vs. Colon Metastasis) | 9 | 0 | 9.14 | |
| | | 15, 16 (Normal Colon vs. Colon Tumor Tissue) | 0 | 9 | | 8.51 |
| 1063 | 403632 | 15, 17 (Normal Colon Tissue vs. Colon Metastasis) | 2 | 11 | | 5.12 |
| 1064 | 390124 | 18, 19 (Normal Colon Tissue vs. Colon Tumor) | 0 | 8 | | 7 |
| | | 15, 17 (Normal Colon Tissue vs. Colon Metastasis) | 6 | 18 | | 2.79 |
| | | 19, 20 (Colon Tumor Tissue vs. Colon Metastasis) | 8 | 0 | 5.98 | |
| 1065 | 390124 | 18, 19 (Normal Colon Tissue vs. Colon Tumor) | 0 | 8 | | 7 |
| | | 19, 20 (Colon Tumor Tissue vs. Colon Metastasis) | 8 | 0 | 5.98 | |
| | | 15, 17 (Normal Colon Tissue vs. Colon Metastasis) | 6 | 18 | | 2.79 |
| 1066 | 422687 | 15, 16 (Normal Colon vs. Colon Tumor Tissue) | 0 | 10 | | 9.46 |
| | | 16, 17 (Colon Tumor Tissue vs. Colon Metastasis) | 10 | 0 | 10.16 | |
| 1067 | 394413 | 18, 19 (Normal Colon Tissue vs. Colon Tumor) | 21 | 2 | 12 | |
| | | 15, 16 (Normal Colon vs. Colon Tumor Tissue) | 113 | 3 | 39.81 | |
| | | 15, 17 (Normal Colon Tissue vs. Colon Metastasis) | 113 | 0 | 121.29 | |
| | | 18, 20 (Normal Colon Tissue vs. Colon Metastasis) | 21 | 0 | 17.95 | |
| 1068 | 549178 | 16, 17 (Colon Tumor Tissue vs. Colon Metastasis) | 0 | 10 | | 9.85 |
| | | 15, 17 (Normal Colon Tissue vs. Colon Metastasis) | 1 | 10 | | 9.32 |
| 1069 | 453079 | 16, 17 (Colon Tumor Tissue vs. Colon Metastasis) | 8 | 1 | 8.12 | |
| 1070 | 463824 | 15, 16 (Normal Colon vs. Colon Tumor Tissue) | 6 | 0 | 6.34 | |
| | | 15, 17(Normal Colon Tissue vs. Colon Metastasis) | 6 | 0 | 6.44 | |
| 1071 | 736595 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1072 | 102655 | 03, 04 (Breast, High Met vs. Breast, Non-Met) | 4 | 33 | | 8.46 |
| | | 15, 17 (Normal Colon Tissue vs. Colon Metastasis) | 14 | 0 | 15.03 | |
| | | 15, 16 (Normal Colon vs. Colon Tumor Tissue) | 14 | 0 | 14.8 | |
| 1073 | 448606 | 15, 17 (Normal Colon Tissue vs. Colon Metastasis) | 3 | 20 | | 6.21 |
| 1074 | 504513 | 15, 17 (Normal Colon Tissue vs. Colon Metastasis) | 6 | 0 | 6.44 | |
| 1075 | 20036 | 15, 17 (Normal Colon Tissue vs. Colon Metastasis) | 1 | 13 | | 12.11 |
| 1076 | 530883 | 15, 16 (Normal Colon vs. Colon Tumor Tissue) | 0 | 7 | | 6.62 |
| 1077 | 447126 | 15, 16 (Normal Colon vs. Colon Tumor Tissue) | 19 | 5 | 4.02 | |
| | | 16, 17 (Colon Tumor Tissue vs. Colon Metastasis) | 5 | 20 | | 3.94 |
| 1078 | 556561 | 15, 17 (Normal Colon Tissue vs. Colon Metastasis) | 0 | 10 | | 9.32 |
| | | 16, 17 (Colon Tumor Tissue vs. Colon Metastasis) | 1 | 10 | | 9.85 |
| 1079 | 455096 | 16, 17 (Colon Tumor Tissue vs. Colon Metastasis) | 2 | 10 | | 4.92 |
| | | 15, 17 (Normal Colon Tissue vs. Colon Metastasis) | 1 | 10 | | 9.32 |
| 1080 | 549320 | 15, 17 (Normal Colon Tissue vs. Colon Metastasis) | 0 | 7 | | 6.52 |
| 1081 | 560984 | 16, 17 (Colon Tumor Tissue vs. Colon Metastasis) | 0 | 6 | | 5.91 |
| 1082 | 450791 | 15, 16 (Normal Colon vs. Colon Tumor Tissue) | 18 | 6 | 3.17 | |
| | | 15, 17 (Normal Colon Tissue vs. Colon Metastasis) | 18 | 3 | 6.44 | |
| 1083 | 16556 | 15, 16 (Normal Colon vs. Colon Tumor Tissue) | 12 | 3 | 4.23 | |
| 1084 | 402707 | 15, 17 (Normal Colon Tissue vs. Colon Metastasis) | 1 | 9 | | 8.38 |
| 1085 | 557903 | 15, 16 (Normal Colon vs. Colon Tumor Tissue) | 0 | 9 | | 8.51 |
| 1086 | 451243 | 15, 17 (Normal Colon Tissue vs. Colon Metastasis) | 0 | 7 | | 6.52 |
| | | 15, 16 (Normal Colon vs. Colon Tumor Tissue) | 0 | 6 | | 5.68 |
| 1087 | 452506 | 15, 16 (Normal Colon vs. Colon Tumor Tissue) | 7 | 0 | 7.4 | |
| | | 16, 17 (Colon Tumor Tissue vs. Colon Metastasis) | 0 | 6 | | 5.91 |
| 1088 | 554703 | 15, 17 (Normal Colon Tissue vs. Colon Metastasis) | 0 | 9 | | 8.38 |
| | | 16, 17 (Colon Tumor Tissue vs. Colon Metastasis) | 1 | 9 | | 8.86 |
| 1089 | 449580 | 15, 16 (Normal Colon vs. Colon Tumor Tissue) | 16 | 4 | 4.23 | |
| 1090 | 3316 | 16, 17 (Colon Tumor Tissue vs. Colon Metastasis) | 0 | 6 | | 5.91 |
| 1091 | 97507 | 15, 16 (Normal Colon vs. Colon Tumor Tissue) | 1 | 9 | | 8.51 |
| 1092 | 556216 | 16, 17 (Colon Tumor Tissue vs. Colon Metastasis) | 0 | 7 | | 6.89 |
| | | 15, 17 (Normal Colon Tissue vs. Colon Metastasis) | 0 | 7 | | 6.52 |
| 1093 | 185401 | 03, 04 (Breast, High Met vs. Breast, Non-Met) | 0 | 63 | | 64.57 |
| 1094 | 3758 | 03, 04 (Breast, High Met vs. Breast, Non-Met) | 17 | 5 | 3.32 | |
| 1095 | 95700 | 03, 04 (Breast, High Met vs. Breast, Non-Met) | 1 | 21 | | 21.52 |
| 1096 | 2478 | 03, 04 (Breast, High Met vs. Breast, Non-Met) | 17 | 5 | 3.32 | |
| 1097 | 550267 | 15, 17 (Normal Colon Tissue vs. Colon Metastasis) | 15 | 4 | 4.03 | |
| | | 15, 16 (Normal Colon vs. Colon Tumor Tissue) | 15 | 1 | 15.85 | |
| 1098 | 185652 | 03, 04 (Breast, High Met vs. Breast, Non-Met) | 0 | 7 | | 7.17 |
| 1099 | 55798 | 03, 04 (Breast, High Met vs. Breast, Non-Met) | 0 | 9 | | 9.22 |
| 1100 | 5078 | 15, 16 (Normal Colon vs. Colon Tumor Tissue) | 0 | 6 | | 5.68 |
| 1101 | 9784 | 03, 04 (Breast, High Met vs. Breast, Non-Met) | 0 | 6 | | 6.15 |
| 1102 | 2245 | 03, 04 (Breast, High Met vs. Breast, Non-Met) | 13 | 1 | 12.68 | |
| | | 01, 02 (Colon, High Met vs. Colon, Low Met) | 12 | 27 | | 2.44 |
| 1103 | 11606 | 03, 04 (Breast, High Met vs. Breast, Non-Met) | 6 | 0 | 5.85 | |
| 1104 | 2245 | 03, 04 (Breast, High Met vs. Breast, Non-Met) | 13 | 1 | 12.68 | |
| | | 01, 02 (Colon, High Met vs. Colon, Low Met) | 12 | 27 | | 2.44 |
| 1105 | 551172 | 15, 16 (Normal Colon vs. Colon Tumor Tissue) | 1 | 10 | | 9.46 |
| 1106 | 729175 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1107 | 6317 | 01, 02 (Colon, High Met vs. Colon, Low Met) | 0 | 6 | | 6.5 |
| 1108 | 2478 | 03, 04 (Breast, High Met vs. Breast, Non-Met) | 17 | 5 | 3.32 | |
| 1109 | 4727 | 03, 04 (Breast, High Met vs. Breast, Non-Met) | 19 | 0 | 18.54 | |
| 1110 | 185598 | 03, 04 (Breast, High Met vs. Breast, Non-Met) | 0 | 8 | | 8.2 |
| 1111 | 736349 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1113 | 189561 | 15, 16 (Normal Colon vs. Colon Tumor Tissue) | 0 | 9 | | 8.51 |
| | | 08, 09 (Lung, High Met vs. Lung, Low Met) | 1 | 14 | | 10.02 |
| | | 15, 17 (Normal Colon Tissue vs. Colon Metastasis) | 0 | 9 | | 8.38 |
| 1114 | 728131 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1115 | 560984 | 16, 17 (Colon Tumor Tissue vs. Colon Metastasis) | 0 | 6 | | 5.91 |
| 1116 | 549945 | 15, 17 (Normal Colon Tissue vs. Colon Metastasis) | 12 | 2 | 6.44 | |
| | | 15, 16 (Normal Colon vs. Colon Tumor Tissue) | 12 | 2 | 6.34 | |
| 1117 | 554785 | 16, 17 (Colon Tumor Tissue vs. Colon Metastasis) | 0 | 8 | | 7.88 |
| | | 15, 17 (Normal Colon Tissue vs. Colon Metastasis) | 0 | 8 | | 7.45 |
| 1118 | 554785 | 15, 17 (Normal Colon Tissue vs. Colon Metastasis) | 0 | 8 | | 7.45 |
| | | 16, 17 (Colon Tumor Tissue vs. Colon Metastasis) | 0 | 8 | | 7.88 |
| 1119 | 551235 | 16, 17 (Colon Tumor Tissue vs. Colon Metastasis) | 0 | 7 | | 6.89 |
| 1120 | 2634 | 03, 04 (Breast, High Met vs. Breast, Non-Met) | 48 | 0 | 46.83 | |
| 1121 | 548858 | 16, 17 (Colon Tumor Tissue vs. Colon Metastasis) | 0 | 11 | | 10.83 |
| 1122 | 15625 | 15, 17 (Normal Colon Tissue vs. Colon Metastasis) | 17 | 0 | 18.25 | |
| | | 15, 16 (Normal Colon vs. Colon Tumor Tissue) | 17 | 1 | 17.97 | |
| 1123 | 649259 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1124 | 550267 | 15, 16 (Normal Colon vs. Colon Tumor Tissue) | 15 | 1 | 15.85 | |
| | | 15, 17 (Normal Colon Tissue vs. Colon Metastasis) | 15 | 4 | 4.03 | |
| 1125 | 7436 | 03, 04 (Breast, High Met vs. Breast, Non-Met) | 9 | 1 | 8.78 | |
| 1126 | 451794 | 15, 16 (Normal Colon vs. Colon Tumor Tissue) | 15 | 0 | 15.85 | |
| | | 15, 17 (Normal Colon Tissue vs. Colon Metastasis) | 15 | 1 | 16.1 | |
| 1127 | 5744 | 01, 02 (Colon, High Met vs. Colon, Low Met) | 0 | 6 | | 6.5 |
| 1128 | 3516 | 01, 02 (Colon, High Met vs. Colon, Low Met) | 5 | 17 | | 3.69 |
| 1129 | 730555 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1130 | 3085 | 03, 04 (Breast, High Met vs. Breast, Non-Met) | 2 | 11 | | 5.64 |
| 1131 | 638854 | 15, 16 (Normal Colon vs. Colon Tumor Tissue) | 32 | 11 | 3.07 | |
| | | 15, 17 (Normal Colon Tissue vs. Colon Metastasis) | 32 | 0 | 34.35 | |
| | | 16, 17 (Colon Tumor Tissue vs. Colon Metastasis) | 11 | 0 | 11.17 | |
| 1132 | 7379 | 01, 02 (Colon, High Met vs. Colon, Low Met) | 0 | 6 | | 6.5 |
| 1133 | 185562 | 03, 04 (Breast, High Met vs. Breast, Non-Met) | 0 | 9 | | 9.22 |
| 1134 | 452491 | 15, 16 (Normal Colon vs. Colon Tumor Tissue) | 12 | 3 | 4.23 | |
| 1135 | 646248 | 16, 17 (Colon Tumor Tissue vs. Colon Metastasis) | 6 | 0 | 6.09 | |
| 1136 | 6056 | 03, 04 (Breast, High Met vs. Breast, Non-Met) | 7 | 0 | 6.83 | |
| 1137 | 643103 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 1138 | 6923 | 03, 04 (Breast, High Met vs. Breast, Non-Met) | 6 | 0 | 5.85 | |
| 1139 | 6923 | 03, 04 (Breast, High Met vs. Breast, Non-Met) | 6 | 0 | 5.85 | |
| 1140 | 901 | 03, 04 (Breast, High Met vs. Breast, Non-Met) | 56 | 5 | 10.93 | |
| 1141 | 901 | 03, 04 (Breast, High Met vs. Breast, Non-Met) | 56 | 5 | 10.93 | |
| 1142 | 367 | 08, 09 (Lung, High Met vs. Lung, Low Met) | 30 | 99 | | 2.36 |
| | | 15, 16 (Normal Colon vs. Colon Tumor Tissue) | 13 | 4 | 3.44 | |
| | | 03, 04 (Breast, High Met vs. Breast, Non-Met) | 105 | 24 | 4.27 | |
| | | 01, 02 (Colon, High Met vs. Colon, Low Met) | 19 | 97 | | 5.53 |
| 1143 | 4043 | 01, 02 (Colon, High Met vs. Colon, Low Met) | 1 | 9 | | 9.76 |
| 1144 | 3299 | 08, 09 (Lung, High Met vs. Lung, Low Met) | 10 | 1 | 13.97 | |
| | | 03, 04 (Breast, High Met vs. Breast, Non-Met) | 20 | 3 | 6.5 | |
| 1145 | 11881 | 03, 04 (Breast, High Met vs. Breast, Non-Met) | 6 | 0 | 5.85 | |
| 1146 | 9113 | 01, 02 (Colon, High Met vs. Colon, Low Met) | 0 | 7 | | 7.59 |
| 1147 | 185460 | 03, 04 (Breast, High Met vs. Breast, Non-Met) | 0 | 16 | | 16.4 |
| 1148 | 185716 | 03, 04 (Breast, High Met vs. Breast, Non-Met) | 0 | 6 | | 6.15 |
| 1149 | 5753 | 01, 02 (Colon, High Met vs. Colon, Low Met) | 0 | 10 | | 10.84 |
| 1150 | 24939 | 15, 16 (Normal Colon vs. Colon Tumor Tissue) | 0 | 8 | | 7.57 |
| 1151 | 649684 | 15, 17 (Normal Colon Tissue vs. Colon Metastasis) | 10 | 0 | 10.73 | |
| 1152 | 642109 | 15, 17 (Normal Colon Tissue vs. Colon Metastasis) | 6 | 0 | 6.44 | |
| 1153 | 15035 | 15, 16 (Normal Colon vs. Colon Tumor Tissue) | 8 | 0 | 8.46 | |
| | | 15, 17 (Normal Colon Tissue vs. Colon Metastasis) | 8 | 1 | 8.59 | |
| 1154 | 649354 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1155 | 4465 | 01, 02 (Colon, High Met vs. Colon, Low Met) | 4 | 14 | | 3.79 |
| 1156 | 647952 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1157 | 455601 | 16, 17 (Colon Tumor Tissue vs. Colon Metastasis) | 8 | 0 | 8.12 | |
| | | 15, 17 (Normal Colon Tissue vs. Colon Metastasis) | 10 | 0 | 10.73 | |
| 1158 | 641901 | 15, 17 (Normal Colon Tissue vs. Colon Metastasis) | 6 | 0 | 6.44 | |
| 1159 | 446878 | 16, 17 (Colon Tumor Tissue vs. Colon Metastasis) | 1 | 12 | | 11.82 |
| 1160 | 7436 | 03, 04 (Breast, High Met vs. Breast, Non-Met) | 9 | 1 | 8.78 | |
| 1161 | 2245 | 01, 02 (Colon, High Met vs. Colon, Low Met) | 12 | 27 | | 2.44 |
| | | 03, 04 (Breast, High Met vs. Breast, Non-Met) | 13 | 1 | 12.68 | |
| 1162 | 3531 | 01, 02 (Colon, High Met vs. Colon, Low Met) | 3 | 17 | | 6.14 |
| 1163 | 9625 | 03, 04 (Breast, High Met vs. Breast, Non-Met) | 1 | 12 | | 12.3 |
| 1164 | 727489 | 16, 17 (Colon Tumor Tissue vs. Colon Metastasis) | 9 | 0 | 9.14 | |
| | | 15, 16 (Normal Colon vs. Colon Tumor Tissue) | 0 | 9 | | 8.51 |
| 1165 | 159925 | 03, 04 (Breast, High Met vs. Breast, Non-Met) | 1 | 22 | | 22.55 |
| 1166 | 645210 | 15, 16 (Normal Colon vs. Colon Tumor Tissue) | 10 | 1 | 10.57 | |
| | | 15, 17 (Normal Colon Tissue vs. Colon Metastasis) | 10 | 0 | 10.73 | |
| 1167 | 157629 | 03, 04 (Breast, High Met vs. Breast, Non-Met) | 3 | 18 | | 6.15 |
| 1168 | 8375 | 03, 04 (Breast, High Met vs. Breast, Non-Met) | 7 | 0 | 6.83 | |
| 1169 | 4319 | 03, 04 (Breast, High Met vs. Breast, Non-Met) | 14 | 0 | 13.66 | |
| | | 01, 02 (Colon, High Met vs. Colon, Low Met) | 0 | 6 | | 6.5 |
| 1170 | 4045 | 01, 02 (Colon, High Met vs. Colon, Low Met) | 2 | 11 | | 5.96 |
| | | 03, 04 (Breast, High Met vs. Breast, Non-Met) | 10 | 1 | 9.76 | |
| 1171 | 185642 | 03, 04 (Breast, High Met vs. Breast, Non-Met) | 0 | 7 | | 7.17 |
| 1172 | 7436 | 03, 04 (Breast, High Met vs. Breast, Non-Met) | 9 | 1 | 8.78 | |
| 1173 | 3531 | 01, 02 (Colon, High Met vs. Colon, Low Met) | 3 | 17 | | 6.14 |
| 1174 | 644776 | 16, 17 (Colon Tumor Tissue vs. Colon Metastasis) | 9 | 0 | 9.14 | |
| 1175 | 8354 | 03, 04 (Breast, High Met vs. Breast, Non-Met) | 7 | 0 | 6.83 | |
| 1176 | 2099 | 03, 04 (Breast, High Met vs. Breast, Non-Met) | 27 | 7 | 3.76 | |
| 1177 | 449956 | 16, 17 (Colon Tumor Tissue vs. Colon Metastasis) | 10 | 1 | 10.16 | |
| 1178 | 649106 | 15, 17 (Normal Colon Tissue vs. Colon Metastasis) | 7 | 0 | 7.51 | |
| 1179 | 452414 | 15, 16 (Normal Colon vs. Colon Tumor Tissue) | 17 | 0 | 17.97 | |
| | | 15, 17 (Normal Colon Tissue vs. Colon Metastasis) | 17 | 0 | 18.25 | |
| 1180 | 732712 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1181 | 185562 | 03, 04 (Breast, High Met vs. Breast, Non-Met) | 0 | 9 | | 9.22 |
| 1182 | 3516 | 01, 02 (Colon, High Met vs. Colon, Low Met) | 5 | 17 | | 3.69 |
| 1183 | 185562 | 03, 04 (Breast, High Met vs. Breast, Non-Met) | 0 | 9 | | 9.22 |
| 1184 | 185460 | 03, 04 (Breast, High Met vs. Breast, Non-Met) | 0 | 16 | | 16.4 |
| 1185 | 10947 | 03, 04 (Breast, High Met vs. Breast, Non-Met) | 8 | 0 | 7.81 | |
| 1186 | 452856 | 15, 16 (Normal Colon vs. Colon Tumor Tissue) | 6 | 0 | 6.34 | |
| 1187 | 558767 | 15, 16 (Normal Colon vs. Colon Tumor Tissue) | 8 | 1 | 8.46 | |
| 1188 | 15035 | 15, 17 (Normal Colon Tissue vs. Colon Metastasis) | 8 | 1 | 8.59 | |
| | | 15, 16 (Normal Colon vs. Colon Tumor Tissue) | 8 | 0 | 8.46 | |
| 1189 | 556421 | 15, 16 (Normal Colon vs. Colon Tumor Tissue) | 12 | 1 | 12.68 | |
| 1190 | 7082 | 03, 04 (Breast, High Met vs. Breast, Non-Met) | 10 | 0 | 9.76 | |
| 1191 | 452523 | 15, 17 (Normal Colon Tissue vs. Colon Metastasis) | 10 | 2 | 5.37 | |
| 1192 | 3242 | 01, 02 (Colon, High Met vs. Colon, Low Met) | 2 | 17 | | 9.22 |
| 1193 | 6660 | 01, 02 (Colon, High Met vs. Colon, Low Met) | 7 | 0 | 6.46 | |
| 1194 | 547 | 01, 02 (Colon, High Met vs. Colon, Low Met) | 35 | 67 | | 2.08 |
| | | 03, 04 (Breast, High Met vs. Breast, Non-Met) | 90 | 30 | 2.93 | |
| | | 15, 16 (Normal Colon vs. Colon Tumor Tissue) | 1 | 10 | | 9.46 |
| 1195 | 121213 | 16, 17 (Colon Tumor Tissue vs. Colon Metastasis) | 8 | 0 | 8.12 | |
| 1196 | 4378 | 16, 17 (Colon Tumor Tissue vs. Colon Metastasis) | 2 | 11 | | 5.42 |
| | | 03, 04 (Breast, High Met vs. Breast, Non-Met) | 1 | 8 | | 8.2 |
| | | 15, 16 (Normal Colon vs. Colon Tumor Tissue) | 11 | 2 | 5.81 | |
| 1197 | 185554 | 03, 04 (Breast, High Met vs. Breast, Non-Met) | 0 | 9 | | 9.22 |
| 1198 | 185482 | 03, 04 (Breast, High Met vs. Breast, Non-Met) | 0 | 13 | | 13.32 |
| 1200 | 66017 | 03, 04 (Breast, High Met vs. Breast, Non-Met) | 0 | 8 | | 8.2 |
| 1201 | 403111 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1202 | 3224 | 01, 02 (Colon, High Met vs. Colon, Low Met) | 17 | 2 | 7.84 | |
| 1203 | 966 | 01, 02 (Colon, High Met vs. Colon, Low Met) | 22 | 47 | | 2.32 |
| | | 03, 04 (Breast, High Met vs. Breast, Non-Met) | 50 | 19 | 2.57 | |
| 1204 | 3639 | 03, 04 (Breast, High Met vs. Breast, Non-Met) | 12 | 3 | 3.9 | |
| 1205 | 5388 | 03, 04 (Breast, High Met vs. Breast, Non-Met) | 5 | 21 | | 4.3 |
| 1206 | 3299 | 08, 09 (Lung, High Met vs. Lung, Low Met) | 10 | 1 | 13.97 | |
| | | 03, 04 (Breast, High Met vs. Breast, Non-Met) | 20 | 3 | 6.5 | |
| 1207 | 23760 | 03, 04 (Breast, High Met vs. Breast, Non-Met) | 4 | 34 | | 8.71 |
| 1208 | 729384 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1209 | 46559 | 03, 04 (Breast, High Met vs. Breast, Non-Met) | 2 | 30 | | 15.37 |
| | | 15, 16 (Normal Colon vs. Colon Tumor Tissue) | 10 | 2 | 5.28 | |
| 1210 | 449750 | 15, 17 (Normal Colon Tissue vs. Colon Metastasis) | 4 | 28 | | 6.52 |
| | | 16, 17 (Colon Tumor Tissue vs. Colon Metastasis) | 1 | 28 | | 27.57 |
| 1211 | 735936 | 16, 17 (Colon Tumor Tissue vs. Colon Metastasis) | 9 | 0 | 9.14 | |
| | | 15, 16 (Normal Colon vs. Colon Tumor Tissue) | 0 | 9 | | 8.51 |
| 1212 | 607430 | 15, 16 (Normal Colon vs. Colon Tumor Tissue) | 6 | 0 | 6.34 | |
| 1213 | 452856 | 15, 16 (Normal Colon vs. Colon Tumor Tissue) | 6 | 0 | 6.34 | |
| 1214 | 557903 | 15, 16 (Normal Colon vs. Colon Tumor Tissue) | 0 | 9 | | 8.51 |
| 1215 | 453112 | 16, 17 (Colon Tumor Tissue vs. Colon Metastasis) | 3 | 13 | | 4.27 |
| | | 15, 17 (Normal Colon Tissue vs. Colon Metastasis) | 3 | 13 | | 4.04 |
| 1216 | 645900 | 23, 24 (Normal Lung Tissue vs. Lung Tumor Tissue) | 7 | 0 | 7.07 | |
| | | 15, 17 (Normal Colon Tissue vs. Colon Metastasis) | 9 | 0 | 9.66 | |
| | | 16, 17 (Colon Tumor Tissue vs. Colon Metastasis) | 9 | 0 | 9.14 | |
| 1217 | 415114 | 15, 16 (Normal Colon vs. Colon Tumor Tissue) | 10 | 0 | 10.57 | |
| 1218 | 418763 | 15, 17 (Normal Colon Tissue vs. Colon Metastasis) | 9 | 0 | 9.66 | |
| 1219 | 2245 | 03, 04 (Breast, High Met vs. Breast, Non-Met) | 13 | 1 | 12.68 | |
| | | 01, 02 (Colon, High Met vs. Colon, Low Met) | 12 | 27 | | 2.44 |
| 1220 | 403668 | 16, 17 (Colon Tumor Tissue vs. Colon Metastasis) | 13 | 3 | 4.4 | |
| | | 15, 16 (Normal Colon vs. Colon Tumor Tissue) | 2 | 13 | | 6.15 |
| 1221 | 15427 | 15, 17 (Normal Colon Tissue vs. Colon Metastasis) | 0 | 7 | | 6.52 |
| | | 15, 16 (Normal Colon vs. Colon Tumor Tissue) | 0 | 8 | | 7.57 |
| 1222 | 555714 | 15, 16 (Normal Colon vs. Colon Tumor Tissue) | 23 | 11 | 2.21 | |
| | | 15, 17 (Normal Colon Tissue vs. Colon Metastasis) | 23 | 7 | 3.53 | |
| 1223 | 555830 | 15, 17 (Normal Colon Tissue vs. Colon Metastasis) | 16 | 2 | 8.59 | |
| | | 15, 16 (Normal Colon vs. Colon Tumor Tissue) | 16 | 2 | 8.46 | |
| 1224 | 4620 | 03, 04 (Breast, High Met vs. Breast, Non-Met) | 7 | 0 | 6.83 | |
| 1225 | 171511 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1226 | 451401 | 15, 17 (Normal Colon Tissue vs. Colon Metastasis) | 9 | 1 | 9.66 | |
| 1227 | 447501 | 15, 17 (Normal Colon Tissue vs. Colon Metastasis) | 15 | 3 | 5.37 | |
| 1228 | 460445 | 16, 17 (Colon Tumor Tissue vs. Colon Metastasis) | 0 | 6 | | 5.91 |
| 1229 | 375814 | 15, 17 (Normal Colon Tissue vs. Colon Metastasis) | 8 | 1 | 8.59 | |
| | | 15, 16 (Normal Colon vs. Colon Tumor Tissue) | 8 | 0 | 8.46 | |
| 1230 | 449356 | 15, 17 (Normal Colon Tissue vs. Colon Metastasis) | 7 | 21 | | 2.79 |
| | | 16, 17 (Colon Tumor Tissue vs. Colon Metastasis) | 4 | 21 | | 5.17 |
| 1231 | 468736 | 16, 17 (Colon Tumor Tissue vs. Colon Metastasis) | 0 | 6 | | 5.91 |
| 1232 | 548858 | 16, 17 (Colon Tumor Tissue vs. Colon Metastasis) | 0 | 11 | | 10.83 |
| 1233 | 3693 | 03, 04 (Breast, High Met vs. Breast, Non-Met) | 0 | 6 | | 6.15 |
| 1234 | 642973 | 16, 17 (Colon Tumor Tissue vs. Colon Metastasis) | 7 | 0 | 7.11 | |
| 1235 | 561180 | 15, 16 (Normal Colon vs. Colon Tumor Tissue) | 11 | 2 | 5.81 | |
| 1236 | 453708 | 16, 17 (Colon Tumor Tissue vs. Colon Metastasis) | 1 | 19 | | 18.71 |
| | | 15, 17 (Normal Colon Tissue vs. Colon Metastasis) | 1 | 19 | | 17.7 |
| 1237 | 645305 | 15, 17 (Normal Colon Tissue vs. Colon Metastasis) | 7 | 0 | 7.51 | |
| 1238 | 463487 | 15, 17 (Normal Colon Tissue vs. Colon Metastasis) | 8 | 1 | 8.59 | |
| 1239 | 11131 | 01, 02 (Colon, High Met vs. Colon, Low Met) | 0 | 6 | | 6.5 |
| 1240 | 561807 | 16, 17 (Colon Tumor Tissue vs. Colon Metastasis) | 1 | 8 | | 7.88 |
| 1241 | 452800 | 15, 17 (Normal Colon Tissue vs. Colon Metastasis) | 3 | 13 | | 4.04 |
| 1242 | 372960 | 15, 17 (Normal Colon Tissue vs. Colon Metastasis) | 6 | 18 | | 2.79 |
| | | 15, 16 (Normal Colon vs. Colon Tumor Tissue) | 6 | 33 | | 5.2 |
| 1243 | 449317 | 15, 17 (Normal Colon Tissue vs. Colon Metastasis) | 5 | 20 | | 3.73 |
| | | 16, 17 (Colon Tumor Tissue vs. Colon Metastasis) | 8 | 20 | | 2.46 |
| 1244 | 730759 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1245 | 9113 | 01, 02 (Colon, High Met vs. Colon, Low Met) | 0 | 7 | | 7.59 |
| 1246 | 630259 | 15, 16 (Normal Colon vs. Colon Tumor Tissue) | 7 | 0 | 7.4 | |
| 1247 | 3516 | 01, 02 (Colon, High Met vs. Colon, Low Met) | 5 | 17 | | 3.69 |
| 1248 | 447494 | 15, 16 (Normal Colon vs. Colon Tumor Tissue) | 26 | 8 | 3.44 | |
| 1249 | 554500 | 15, 16 (Normal Colon vs. Colon Tumor Tissue) | 6 | 0 | 6.34 | |
| 1250 | 639662 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 1251 | 421 | 15, 17 (Normal Colon Tissue vs. Colon Metastasis) | 0 | 7 | | 6.52 |
| 1252 | 736014 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1253 | 643061 | 15, 17 (Normal Colon Tissue vs. Colon Metastasis) | 8 | 0 | 8.59 | |
| | | 15, 16 (Normal Colon vs. Colon Tumor Tissue) | 8 | 1 | 8.46 | |
| 1254 | 9113 | 01, 02 (Colon, High Met vs. Colon, Low Met) | 0 | 7 | | 7.59 |
| 1255 | 650856 | 16, 17 (Colon Tumor Tissue vs. Colon Metastasis) | 6 | 0 | 6.09 | |
| 1256 | 476223 | 23, 24 (Normal Lung Tissue vs. Lung Tumor Tissue) | 2 | 19 | | 9.4 |
| | | 15, 17 (Normal Colon Tissue vs. Colon Metastasis) | 7 | 0 | 7.51 | |
| 1257 | 737088 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1258 | 449512 | 15, 17 (Normal Colon Tissue vs. Colon Metastasis) | 4 | 14 | | 3.26 |
| | | 16, 17 (Colon Tumor Tissue vs. Colon Metastasis) | 1 | 14 | | 13.79 |
| 1259 | 449457 | 15, 17 (Normal Colon Tissue vs. Colon Metastasis) | 0 | 7 | | 6.52 |
| 1260 | 521901 | 16, 17 (Colon Tumor Tissue vs. Colon Metastasis) | 0 | 6 | | 5.91 |
| 1261 | 175799 | 15, 16 (Normal Colon vs. Colon Tumor Tissue) | 14 | 3 | 4.93 | |
| | | 23, 24 (Normal Lung Tissue vs. Lung Tumor Tissue) | 1 | 9 | | 8.91 |
| 1262 | 550108 | 15, 16 (Normal Colon vs. Colon Tumor Tissue) | 6 | 19 | | 3 |
| | | 15, 17 (Normal Colon Tissue vs. Colon Metastasis) | 6 | 37 | | 5.75 |
| 1263 | 203605 | 15, 16 (Normal Colon vs. Colon Tumor Tissue) | 17 | 7 | 2.57 | |
| 1264 | 450429 | 15, 17 (Normal Colon Tissue vs. Colon Metastasis) | 13 | 1 | 13.95 | |
| | | 16, 17 (Colon Tumor Tissue vs. Colon Metastasis) | 10 | 1 | 10.16 | |
| 1265 | 2478 | 03, 04 (Breast, High Met vs. Breast, Non-Met) | 17 | 5 | 3.32 | |
| 1266 | 644099 | 15, 17 (Normal Colon Tissue vs. Colon Metastasis) | 6 | 0 | 6.44 | |
| 1267 | 552614 | 16, 17 (Colon Tumor Tissue vs. Colon Metastasis) | 0 | 7 | | 6.89 |
| 1268 | 452523 | 15, 17 (Normal Colon Tissue vs. Colon Metastasis) | 10 | 2 | 5.37 | |
| 1269 | 446789 | 15, 16 (Normal Colon vs. Colon Tumor Tissue) | 16 | 5 | 3.38 | |
| 1270 | 515631 | 15, 16 (Normal Colon vs. Colon Tumor Tissue) | 0 | 6 | | 5.68 |
| 1271 | 452523 | 15, 17 (Normal Colon Tissue vs. Colon Metastasis) | 10 | 2 | 5.37 | |
| 1272 | 640116 | 15, 17 (Normal Colon Tissue vs. Colon Metastasis) | 19 | 0 | 20.39 | |
| | | 15, 16 (Normal Colon vs. Colon Tumor Tissue) | 19 | 4 | 5.02 | |
| 1273 | 9113 | 01, 02 (Colon, High Met vs. Colon, Low Met) | 0 | 7 | | 7.59 |
| 1274 | 562221 | 15, 16 (Normal Colon vs. Colon Tumor Tissue) | 8 | 0 | 8.46 | |
| 1275 | 455972 | 15, 16 (Normal Colon vs. Colon Tumor Tissue) | 0 | 6 | | 5.68 |
| 1276 | 449137 | 15, 16 (Normal Colon vs. Colon Tumor Tissue) | 3 | 18 | | 5.68 |
| 1277 | 5078 | 15, 16 (Normal Colon vs. Colon Tumor Tissue) | 0 | 6 | | 5.68 |
| 1278 | 5078 | 15, 16 (Normal Colon vs. Colon Tumor Tissue) | 0 | 6 | | 5.68 |
| 1279 | 4016 | 03, 04 (Breast, High Met vs. Breast, Non-Met) | 0 | 6 | | 6.15 |
| | | 01, 02 (Colon, High Met vs. Colon, Low Met) | 5 | 14 | | 3.04 |
| 1280 | 403111 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1281 | 562292 | 16, 17 (Colon Tumor Tissue vs. Colon Metastasis) | 0 | 6 | | 5.91 |
| 1282 | 403111 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1283 | 403111 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1284 | 500959 | 15, 17 (Normal Colon Tissue vs. Colon Metastasis) | 8 | 0 | 8.59 | |
| | | 15, 16 (Normal Colon vs. Colon Tumor Tissue) | 8 | 0 | 8.46 | |
| 1285 | 763 | 01, 02 (Colon, High Met vs. Colon, Low Met) | 29 | 77 | | 2.88 |
| | | 03, 04 (Breast, High Met vs. Breast, Non-Met) | 42 | 10 | 4.1 | |
| 1286 | 763 | 03, 04 (Breast, High Met vs. Breast, Non-Met) | 42 | 10 | 4.1 | |
| | | 01, 02 (Colon, High Met vs. Colon, Low Met) | 29 | 77 | | 2.88 |
| 1287 | 500959 | 15, 17 (Normal Colon Tissue vs. Colon Metastasis) | 8 | 0 | 8.59 | |
| | | 15, 16 (Normal Colon vs. Colon Tumor Tissue) | 8 | 0 | 8.46 | |
| 1288 | 452071 | 15, 17 (Normal Colon Tissue vs. Colon Metastasis) | 109 | 0 | 117 | |
| | | 18, 20 (Normal Colon Tissue vs. Colon Metastasis) | 14 | 0 | 11.97 | |
| | | 15, 16 (Normal Colon vs. Colon Tumor Tissue) | 109 | 1 | 115.21 | |
| | | 18, 19 (Normal Colon Tissue vs. Colon Tumor) | 14 | 0 | 16 | |
| 1289 | 468672 | 15, 16 (Normal Colon vs. Colon Tumor Tissue) | 6 | 0 | 6.34 | |
| | | 16, 17 (Colon Tumor Tissue vs. Colon Metastasis) | 0 | 8 | | 7.88 |
| 1290 | 455492 | 15, 16 (Normal Colon vs. Colon Tumor Tissue) | 8 | 1 | 8.46 | |
| 1291 | 639667 | 16, 17 (Colon Tumor Tissue vs. Colon Metastasis) | 9 | 0 | 9.14 | |
| 1292 | 549829 | 16, 17 (Colon Tumor Tissue vs. Colon Metastasis) | 0 | 6 | | 5.91 |
| 1293 | 553158 | 16, 17 (Colon Tumor Tissue vs. Colon Metastasis) | 1 | 8 | | 7.88 |
| 1294 | 561485 | 15, 17 (Normal Colon Tissue vs. Colon Metastasis) | 0 | 7 | | 6.52 |
| | | 16, 17 (Colon Tumor Tissue vs. Colon Metastasis) | 0 | 7 | | 6.89 |
| 1295 | 639352 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1296 | 451401 | 15, 17 (Normal Colon Tissue vs. Colon Metastasis) | 9 | 1 | 9.66 | |
| 1297 | 643103 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 1298 | 468736 | 16, 17 (Colon Tumor Tissue vs. Colon Metastasis) | 0 | 6 | | 5.91 |
| 1299 | 218416 | 16, 17 (Colon Tumor Tissue vs. Colon Metastasis) | 0 | 6 | | 5.91 |
| 1300 | 447501 | 15, 17 (Normal Colon Tissue vs. Colon Metastasis) | 15 | 3 | 5.37 | |
| 1301 | 558371 | 15, 17 (Normal Colon Tissue vs. Colon Metastasis) | 10 | 2 | 5.37 | |
| | | 15, 16 (Normal Colon vs. Colon Tumor Tissue) | 10 | 2 | 5.28 | |
| 1302 | 561794 | 15, 16 (Normal Colon vs. Colon Tumor Tissue) | 0 | 6 | | 5.68 |
| 1303 | 645065 | 15, 17 (Normal Colon Tissue vs. Colon Metastasis) | 6 | 0 | 6.44 | |
| 1304 | 451269 | 15, 16 (Normal Colon vs. Colon Tumor Tissue) | 13 | 3 | 4.58 | |
| | | 15, 17 (Normal Colon Tissue vs. Colon Metastasis) | 13 | 4 | 3.49 | |
| 1305 | 401553 | 15, 17 (Normal Colon Tissue vs. Colon Metastasis) | 8 | 0 | 8.59 | |
| 1306 | 555276 | 16, 17 (Colon Tumor Tissue vs. Colon Metastasis) | 0 | 6 | | 5.91 |
| 1307 | 551617 | 15, 16 (Normal Colon vs. Colon Tumor Tissue) | 9 | 0 | 9.51 | |
| | | 16, 17 (Colon Tumor Tissue vs. Colon Metastasis) | 0 | 7 | | 6.89 |
| 1308 | 463480 | 16, 17 (Colon Tumor Tissue vs. Colon Metastasis) | 0 | 6 | | 5.91 |
| 1309 | 549178 | 15, 17 (Normal Colon Tissue vs. Colon Metastasis) | 1 | 10 | | 9.32 |
| | | 16, 17 (Colon Tumor Tissue vs. Colon Metastasis) | 0 | 10 | | 9.85 |
| 1310 | 374450 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1311 | 562835 | 15, 17 (Normal Colon Tissue vs. Colon Metastasis) | 0 | 9 | | 8.38 |
| 1312 | 730555 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1313 | 732978 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1314 | 1609 | 01, 02 (Colon, High Met vs. Colon, Low Met) | 3 | 58 | | 20.96 |
| | | 03, 04 (Breast, High Met vs. Breast, Non-Met) | 10 | 2 | 4.88 | |
| | | 15, 17 (Normal Colon Tissue vs. Colon Metastasis) | 15 | 3 | 5.37 | |
| | | 16, 17 (Colon Tumor Tissue vs. Colon Metastasis) | 14 | 3 | 4.74 | |
| 1315 | 18591 | 08, 09 (Lung, High Met vs. Lung, Low Met) | 8 | 0 | 11.18 | |
| | | 15, 16 (Normal Colon vs. Colon Tumor Tissue) | 30 | 8 | 3.96 | |
| 1316 | 553158 | 16, 17 (Colon Tumor Tissue vs. Colon Metastasis) | 1 | 8 | | 7.88 |
| 1317 | 470602 | 16, 17 (Colon Tumor Tissue vs. Colon Metastasis) | 1 | 9 | | 8.86 |
| | | 15, 16 (Normal Colon vs. Colon Tumor Tissue) | 12 | 1 | 12.68 | |
| 1318 | 639662 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 1319 | 644721 | 15, 17 (Normal Colon Tissue vs. Colon Metastasis) | 6 | 0 | 6.44 | |
| 1320 | 453202 | 15, 16 (Normal Colon vs. Colon Tumor Tissue) | 6 | 0 | 6.34 | |
| | | 16, 17 (Colon Tumor Tissue vs. Colon Metastasis) | 0 | 11 | | 10.83 |
| 1321 | 554655 | 15, 16 (Normal Colon vs. Colon Tumor Tissue) | 0 | 6 | | 5.68 |
| 1322 | 641988 | 15, 17 (Normal Colon Tissue vs. Colon Metastasis) | 7 | 0 | 7.51 | |
| 1323 | 453112 | 16, 17 (Colon Tumor Tissue vs. Colon Metastasis) | 3 | 13 | | 4.27 |
| | | 15, 17 (Normal Colon Tissue vs. Colon Metastasis) | 3 | 13 | | 4.04 |
| 1324 | 550694 | 15, 16 (Normal Colon vs. Colon Tumor Tissue) | 21 | 1 | 22.2 | |
| | | 15, 17 (Normal Colon Tissue vs. Colon Metastasis) | 21 | 7 | 3.22 | |
| 1325 | 649106 | 15, 17 (Normal Colon Tissue vs. Colon Metastasis) | 7 | 0 | 7.51 | |
| 1326 | 638973 | 16, 17 (Colon Tumor Tissue vs. Colon Metastasis) | 12 | 0 | 12.19 | |
| | | 15, 17 (Normal Colon Tissue vs. Colon Metastasis) | 7 | 0 | 7.51 | |
| 1327 | 549911 | 16, 17 (Colon Tumor Tissue vs. Colon Metastasis) | 0 | 6 | | 5.91 |
| 1328 | 648774 | 15, 17 (Normal Colon Tissue vs. Colon Metastasis) | 6 | 0 | 6.44 | |
| 1329 | 549911 | 16, 17 (Colon Tumor Tissue vs. Colon Metastasis) | 0 | 6 | | 5.91 |
| 1330 | 639662 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1331 | 560455 | 15, 17 (Normal Colon Tissue vs. Colon Metastasis) | 0 | 7 | | 6.52 |
| 1332 | 735805 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1333 | 732712 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1334 | 446663 | 15, 17 (Normal Colon Tissue vs. Colon Metastasis) | 14 | 32 | | 2.13 |
| | | 16, 17 (Colon Tumor Tissue vs. Colon Metastasis) | 6 | 32 | | 52.5 |
| | | 19, 20 (Colon Tumor Tissue vs. Colon Metastasis) | 0 | 8 | | 10.7 |
| | | 18, 20 (Normal Colon Tissue vs. Colon Metastasis) | 1 | 8 | | 9.36 |
| 1335 | 226324 | 16, 17 (Colon Tumor Tissue vs. Colon Metastasis) | 0 | 6 | | 5.91 |
| 1336 | 453016 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1337 | 550998 | 15, 16 (Normal Colon vs. Colon Tumor Tissue) | 0 | 6 | | 5.68 |
| 1338 | 452414 | 15, 16 (Normal Colon vs. Colon Tumor Tissue) | 17 | 0 | 17.97 | |
| | | 15, 17 (Normal Colon Tissue vs. Colon Metastasis) | 17 | 0 | 18.25 | |
| 1339 | 129535 | 15, 17 (Normal Colon Tissue vs. Colon Metastasis) | 41 | 1 | 44.01 | |
| | | 08, 09 (Lung, High Met vs. Lung, Low Met) | 2 | 22 | | 7.87 |
| | | 15, 16 (Normal Colon vs. Colon Tumor Tissue) | 41 | 5 | 8.67 | |
| 1340 | 447089 | 16, 17 (Colon Tumor Tissue vs. Colon Metastasis) | 1 | 8 | | 7.88 |
| 1341 | 447850 | 16, 17 (Colon Tumor Tissue vs. Colon Metastasis) | 0 | 6 | | 5.91 |
| 1342 | 556216 | 15, 17 (Normal Colon Tissue vs. Colon Metastasis) | 0 | 7 | | 6.52 |
| | | 16, 17 (Colon Tumor Tissue vs. Colon Metastasis) | 0 | 7 | | 6.89 |
| 1343 | 452523 | 15, 17 (Normal Colon Tissue vs. Colon Metastasis) | 10 | 2 | 5.37 | |
| 1344 | 44424 | 16, 17 (Colon Tumor Tissue vs. Colon Metastasis) | 41 | 90 | | 2.16 |
| | | 08, 09 (Lung, High Met vs. Lung, Low Met) | 4 | 40 | | 7.16 |
| | | 15, 17 (Normal Colon Tissue vs. Colon Metastasis) | 25 | 90 | | 3.35 |
| 1345 | 648872 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1346 | 451636 | 16, 17 (Colon Tumor Tissue vs. Colon Metastasis) | 1 | 8 | | 7.88 |
| 1347 | 5078 | 15, 16 (Normal Colon vs. Colon Tumor Tissue) | 0 | 6 | | 5.68 |
| 1348 | 403111 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1349 | 648959 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 1350 | 380291 | 15, 16 (Normal Colon vs. Colon Tumor Tissue) | 6 | 0 | 6.34 | |
| 1351 | 380291 | 15, 16 (Normal Colon vs. Colon Tumor Tissue) | 6 | 0 | 6.34 | |
| 1352 | 230995 | 15, 16 (Normal Colon vs. Colon Tumor Tissue) | 0 | 7 | | 6.62 |
| 1353 | 562221 | 15, 16 (Normal Colon vs. Colon Tumor Tissue) | 8 | 0 | 8.46 | |
| 1354 | 450959 | 15, 16 (Normal Colon vs. Colon Tumor Tissue) | 1 | 9 | | 8.51 |
| 1355 | 452833 | 15, 17 (Normal Colon Tissue vs. Colon Metastasis) | 0 | 7 | | 6.52 |
| 1356 | 550195 | 16, 17 (Colon Tumor Tissue vs. Colon Metastasis) | 0 | 11 | | 10.83 |
| | | 15, 17 (Normal Colon Tissue vs. Colon Metastasis) | 2 | 11 | | 5.12 |
| 1357 | 448927 | 15, 17 (Normal Colon Tissue vs. Colon Metastasis) | 2 | 12 | | 5.59 |
| 1358 | 551514 | 16, 17 (Colon Tumor Tissue vs. Colon Metastasis) | 1 | 13 | | 12.8 |
| | | 15, 16 (Normal Colon vs. Colon Tumor Tissue) | 8 | 1 | 8.46 | |
| 1359 | 549829 | 16, 17 (Colon Tumor Tissue vs. Colon Metastasis) | 0 | 6 | | 5.91 |
| 1360 | 551514 | 15, 16 (Normal Colon vs. Colon Tumor Tissue) | 8 | 1 | 8.46 | |
| | | 16, 17 (Colon Tumor Tissue vs. Colon Metastasis) | 1 | 13 | | 12.8 |
| 1361 | 561485 | 16, 17 (Colon Tumor Tissue vs. Colon Metastasis) | 0 | 7 | | 6.89 |
| | | 15, 17 (Normal Colon Tissue vs. Colon Metastasis) | 0 | 7 | | 6.52 |
| 1362 | 453846 | 15, 17 (Normal Colon Tissue vs. Colon Metastasis) | 0 | 11 | | 10.25 |
| 1363 | 69863 | 08, 09 (Lung, High Met vs. Lung, Low Met) | 3 | 23 | | 5.49 |
| | | 03, 04 (Breast, High Met vs. Breast, Non-Met) | 3 | 21 | | 7.17 |
| 1364 | 727181 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1365 | 454050 | 15, 17 (Normal Colon Tissue vs. Colon Metastasis) | 8 | 1 | 8.59 | |
| 1366 | 725994 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1367 | 1495 | 03, 04 (Breast, High Met vs. Breast, Non-Met) | 31 | 12 | 2.52 | |
| 1368 | 5665 | 03, 04 (Breast, High Met vs. Breast, Non-Met) | 13 | 0 | 12.68 | |
| 1369 | 5665 | 03, 04 (Breast, High Met vs. Breast, Non-Met) | 13 | 0 | 12.68 | |
| 1370 | 646146 | 16, 17 (Colon Tumor Tissue vs. Colon Metastasis) | 6 | 0 | 6.09 | |
| | | 15, 17 (Normal Colon Tissue vs. Colon Metastasis) | 8 | 0 | 8.59 | |
| 1371 | 8371 | 01, 02 (Colon, High Met vs. Colon, Low Met) | 0 | 6 | | 6.5 |
| 1372 | 73812 | 03, 04 (Breast, High Met vs. Breast, Non-Met) | 0 | 12 | | 12.3 |
| 1373 | 4242 | 03, 04 (Breast, High Met vs. Breast, Non-Met) | 17 | 0 | 16.59 | |
| 1374 | 5482 | 01, 02 (Colon, High Met vs. Colon, Low Met) | 1 | 8 | | 8.67 |
| 1375 | 5474 | 01, 02 (Colon, High Met vs. Colon, Low Met) | 1 | 8 | | 8.67 |
| | | 03, 04 (Breast, High Met vs. Breast, Non-Met) | 5 | 17 | | 3.48 |
| 1376 | 5448 | 01, 02 (Colon, High Met vs. Colon, Low Met) | 1 | 10 | | 10.84 |
| 1377 | 7607 | 15, 17 (Normal Colon Tissue vs. Colon Metastasis) | 13 | 3 | 4.65 | |
| | | 01, 02 (Colon, High Met vs. Colon, Low Met) | 0 | 7 | | 7.59 |
| | | 15, 16 (Normal Colon vs. Colon Tumor Tissue) | 13 | 4 | 3.44 | |
| 1378 | 555928 | 15, 16 (Normal Colon vs. Colon Tumor Tissue) | 0 | 6 | | 5.68 |
| 1379 | 4046 | 01, 02 (Colon, High Met vs. Colon, Low Met) | 4 | 14 | | 3.79 |
| 1380 | 554080 | 16, 17 (Colon Tumor Tissue vs. Colon Metastasis) | 1 | 9 | | 8.86 |
| 1381 | 451092 | 16, 17 (Colon Tumor Tissue vs. Colon Metastasis) | 1 | 9 | | 8.86 |
| 1382 | 551380 | 16, 17 (Colon Tumor Tissue vs. Colon Metastasis) | 4 | 14 | | 3.45 |
| 1383 | 546642 | 16, 17 (Colon Tumor Tissue vs. Colon Metastasis) | 11 | 0 | 11.17 | |
| 1384 | 1764 | 03, 04 (Breast, High Met vs. Breast, Non-Met) | 27 | 4 | 6.59 | |
| | | 01, 02 (Colon, High Met vs. Colon, Low Met) | 25 | 8 | 2.88 | |
| 1385 | 650773 | 15, 17 (Normal Colon Tissue vs. Colon Metastasis) | 6 | 0 | 6.44 | |
| 1386 | 644205 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1387 | 185718 | 03, 04 (Breast, High Met vs. Breast, Non-Met) | 0 | 6 | | 6.15 |
| 1388 | 5538 | 01, 02 (Colon, High Met vs. Colon, Low Met) | 0 | 8 | | 8.67 |
| | | 03, 04 (Breast, High Met vs. Breast, Non-Met) | 6 | 0 | 5.85 | |
| 1389 | 7546 | 01, 02 (Colon, High Met vs. Colon, Low Met) | 8 | 0 | 7.38 | |
| 1390 | 727789 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1391 | 3837 | 03, 04 (Breast, High Met vs. Breast, Non-Met) | 9 | 1 | 8.78 | |
| 1392 | 380477 | 15, 17 (Normal Colon Tissue vs. Colon Metastasis) | 7 | 0 | 7.51 | |
| 1393 | 3299 | 03, 04 (Breast, High Met vs. Breast, Non-Met) | 20 | 3 | 6.5 | |
| | | 08, 09 (Lung, High Met vs. Lung, Low Met) | 10 | 1 | 13.97 | |
| 1394 | 448853 | 15, 16 (Normal Colon vs. Colon Tumor Tissue) | 1 | 10 | | 9.46 |
| 1395 | 736701 | 23, 24 (Normal Lung Tissue vs. Lung Tumor Tissue) | 10 | 0 | 10.1 | |
| | | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1396 | 735296 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1397 | 13666 | 03, 04 (Breast, High Met vs. Breast, Non-Met) | 1 | 9 | | 9.22 |
| 1398 | 732712 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1399 | 3765 | 01, 02 (Colon, High Met vs. Colon, Low Met) | 19 | 6 | 2.92 | |
| 1400 | 185596 | 03, 04 (Breast, High Met vs. Breast, Non-Met) | 0 | 8 | | 8.2 |
| 1401 | 1943 | 03, 04 (Breast, High Met vs. Breast, Non-Met) | 16 | 4 | 3.9 | |
| | | 15, 16 (Normal Colon vs. Colon Tumor Tissue) | 6 | 19 | | 3 |
| | | 01, 02 (Colon, High Met vs. Colon, Low Met) | 9 | 29 | | 3.49 |
| 1402 | 448193 | 16, 17 (Colon Tumor Tissue vs. Colon Metastasis) | 10 | 2 | 5.08 | |
| | | 15, 16 (Normal Colon vs. Colon Tumor Tissue) | 1 | 10 | | 9.46 |
| 1403 | 1793 | 01, 02 (Colon, High Met vs. Colon, Low Met) | 13 | 27 | | 2.25 |
| | | 03, 04 (Breast, High Met vs. Breast, Non-Met) | 35 | 13 | 2.63 | |
| 1404 | 2475 | 03, 04 (Breast, High Met vs. Breast, Non-Met) | 5 | 35 | | 7.17 |
| 1405 | 730866 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 1406 | 730389 | 15, 16 (Normal Colon vs. Colon Tumor Tissue) | 0 | 9 | | 8.51 |
| | | 16, 17 (Colon Tumor Tissue vs. Colon Metastasis) | 9 | 0 | 9.14 | |
| 1407 | 641884 | 15, 17 (Normal Colon Tissue vs. Colon Metastasis) | 8 | 0 | 8.59 | |
| | | 16, 17 (Colon Tumor Tissue vs. Colon Metastasis) | 7 | 0 | 7.11 | |
| 1408 | 463487 | 15, 17 (Normal Colon Tissue vs. Colon Metastasis) | 8 | 1 | 8.59 | |
| 1409 | 5156 | 01, 02 (Colon, High Met vs. Colon, Low Met) | 1 | 8 | | 8.67 |
| 1410 | 728408 | 15, 16 (Normal Colon vs. Colon Tumor Tissue) | 0 | 12 | | 11.35 |
| | | 16, 17 (Colon Tumor Tissue vs. Colon Metastasis) | 12 | 0 | 12.19 | |
| 1411 | 73812 | 03, 04 (Breast, High Met vs. Breast, Non-Met) | 0 | 12 | | 12.3 |
| 1412 | 1662 | 01, 02 (Colon, High Met vs. Colon, Low Met) | 7 | 34 | | 5.27 |
| | | 03, 04 (Breast, High Met vs. Breast, Non-Met) | 31 | 5 | 6.05 | |
| 1413 | 736556 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 1414 | 5240 | 08, 09 (Lung, High Met vs. Lung, Low Met) | 18 | 10 | 2.52 | |
| | | 03, 04 (Breast, High Met vs. Breast, Non-Met) | 12 | 2 | 5.85 | |
| 1415 | 6184 | 01, 02 (Colon, High Met vs. Colon, Low Met) | 0 | 8 | | 8.67 |
| 1416 | 446404 | 18, 19 (Normal Colon Tissue vs. Colon Tumor) | 23 | 0 | 26.28 | |
| | | 15, 17 (Normal Colon Tissue vs. Colon Metastasis) | 73 | 0 | 78.36 | |
| | | 18, 20 (Normal Colon Tissue vs. Colon Metastasis) | 23 | 0 | 19.66 | |
| | | 15, 16 (Normal Colon vs. Colon Tumor Tissue) | 73 | 1 | 77.16 | |
| 1417 | 646825 | 15, 17 (Normal Colon Tissue vs. Colon Metastasis) | 10 | 0 | 10.73 | |
| 1418 | 734929 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1419 | 648851 | 15, 17 (Normal Colon Tissue vs. Colon Metastasis) | 6 | 0 | 6.44 | |
| 1420 | 640135 | 16, 17 (Colon Tumor Tissue vs. Colon Metastasis) | 6 | 0 | 6.09 | |
| | | 15, 17 (Normal Colon Tissue vs. Colon Metastasis) | 8 | 0 | 8.59 | |
| 1421 | 7443 | 03, 04 (Breast, High Met vs. Breast, Non-Met) | 9 | 0 | 8.78 | |
| 1422 | 454050 | 15, 17 (Normal Colon Tissue vs. Colon Metastasis) | 8 | 1 | 8.59 | |
| 1423 | 3765 | 01, 02 (Colon, High Met vs. Colon, Low Met) | 19 | 6 | 2.92 | |
| 1424 | 648320 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1425 | 451269 | 15, 17 (Normal Colon Tissue vs. Colon Metastasis) | 13 | 4 | 3.49 | |
| | | 15, 16 (Normal Colon vs. Colon Tumor Tissue) | 13 | 3 | 4.58 | |
| 1426 | 535208 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1427 | 728115 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1428 | 5240 | 03, 04 (Breast, High Met vs. Breast, Non-Met) | 12 | 2 | 5.85 | |
| | | 08, 09 (Lung, High Met vs. Lung, Low Met) | 18 | 10 | 2.52 | |
| 1429 | 909 | 08, 09 (Lung, High Met vs. Lung, Low Met) | 34 | 4 | 11.88 | |
| | | 03, 04 (Breast, High Met vs. Breast, Non-Met) | 54 | 18 | 2.93 | |
| 1430 | 447697 | 15, 16 (Normal Colon vs. Colon Tumor Tissue) | 2 | 11 | | 5.2 |
| 1431 | 447737 | 15, 16 (Normal Colon vs. Colon Tumor Tissue) | 19 | 6 | 3.35 | |
| 1432 | 651100 | 15, 17 (Normal Colon Tissue vs. Colon Metastasis) | 7 | 0 | 7.51 | |
| 1433 | 735477 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1434 | 3774 | 01, 02 (Colon, High Met vs. Colon, Low Met) | 1 | 12 | | 13.01 |
| 1435 | 646146 | 16, 17 (Colon Tumor Tissue vs. Colon Metastasis) | 6 | 0 | 6.09 | |
| | | 15, 17 (Normal Colon Tissue vs. Colon Metastasis) | 8 | 0 | 8.59 | |
| 1436 | 643931 | 15, 17 (Normal Colon Tissue vs. Colon Metastasis) | 7 | 0 | 7.51 | |
| 1437 | 463487 | 15, 17 (Normal Colon Tissue vs. Colon Metastasis) | 8 | 1 | 8.59 | |
| 1438 | 650097 | 16, 17 (Colon Tumor Tissue vs. Colon Metastasis) | 6 | 0 | 6.09 | |
| 1439 | 554469 | 15, 17 (Normal Colon Tissue vs. Colon Metastasis) | 24 | 3 | 8.59 | |
| | | 15, 16 (Normal Colon vs. Colon Tumor Tissue) | 24 | 8 | 3.17 | |
| 1440 | 476223 | 15, 17 (Normal Colon Tissue vs. Colon Metastasis) | 7 | 0 | 7.51 | |
| | | 23, 24 (Normal Lung Tissue vs. Lung Tumor Tissue) | 2 | 19 | | 9.4 |
| 1441 | 8738 | 03, 04 (Breast, High Met vs. Breast, Non-Met) | 6 | 0 | 5.85 | |
| 1442 | 403978 | 23, 24 (Normal Lung Tissue vs. Lung Tumor Tissue) | 23 | 50 | | 2.15 |
| | | 15, 16 (Normal Colon vs. Colon Tumor Tissue) | 7 | 19 | | 2.57 |
| 1443 | 185539 | 03, 04 (Breast, High Met vs. Breast, Non-Met) | 0 | 10 | | 10.25 |
| 1444 | 451811 | 15, 17 (Normal Colon Tissue vs. Colon Metastasis) | 10 | 2 | 5.37 | |
| 1445 | 140731 | 15, 17 (Normal Colon Tissue vs. Colon Metastasis) | 7 | 0 | 7.51 | |
| 1446 | 734582 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1447 | 463487 | 15, 17 (Normal Colon Tissue vs. Colon Metastasis) | 8 | 1 | 8.59 | |
| 1448 | 558719 | 15, 16 (Normal Colon vs. Colon Tumor Tissue) | 18 | 6 | 3.17 | |
| 1449 | 21669 | 16, 17 (Colon Tumor Tissue vs. Colon Metastasis) | 0 | 7 | | 6.89 |
| 1450 | 470462 | 15, 17 (Normal Colon Tissue vs. Colon Metastasis) | 7 | 0 | 7.51 | |
| 1451 | 3316 | 16, 17 (Colon Tumor Tissue vs. Colon Metastasis) | 0 | 6 | | 5.91 |
| 1452 | 553728 | 16, 17 (Colon Tumor Tissue vs. Colon Metastasis) | 0 | 11 | | 10.83 |
| 1453 | 736014 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1454 | 237288 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1455 | 11141 | 01, 02 (Colon, High Met vs. Colon, Low Met) | 0 | 6 | | 6.5 |
| 1456 | 556421 | 15, 16 (Normal Colon vs. Colon Tumor Tissue) | 12 | 1 | 12.68 | |
| 1457 | 549435 | 15, 16 (Normal Colon vs. Colon Tumor Tissue) | 14 | 2 | 7.4 | |
| 1458 | 448927 | 15, 17 (Normal Colon Tissue vs. Colon Metastasis) | 2 | 12 | | 5.59 |
| 1459 | 379105 | 15, 16 (Normal Colon vs. Colon Tumor Tissue) | 0 | 8 | | 7.57 |
| 1460 | 552614 | 16, 17 (Colon Tumor Tissue vs. Colon Metastasis) | 0 | 7 | | 6.89 |
| 1461 | 470602 | 15, 16 (Normal Colon vs. Colon Tumor Tissue) | 12 | 1 | 12.68 | |
| | | 16, 17 (Colon Tumor Tissue vs. Colon Metastasis) | 1 | 9 | | 8.86 |
| 1462 | 557039 | 16, 17 (Colon Tumor Tissue vs. Colon Metastasis) | 2 | 16 | | 7.88 |
| | | 15, 16 (Normal Colon vs. Colon Tumor Tissue) | 11 | 2 | 5.81 | |
| 1463 | 549864 | 16, 17 (Colon Tumor Tissue vs. Colon Metastasis) | 0 | 6 | | 5.91 |
| 1464 | 449836 | 15, 17 (Normal Colon Tissue vs. Colon Metastasis) | 8 | 1 | 8.59 | |
| 1465 | 554812 | 16, 17 (Colon Tumor Tissue vs. Colon Metastasis) | 1 | 8 | | 7.88 |
| | | 15, 17 (Normal Colon Tissue vs. Colon Metastasis) | 0 | 8 | | 7.45 |
| 1466 | 3316 | 16, 17 (Colon Tumor Tissue vs. Colon Metastasis) | 0 | 6 | | 5.91 |
| 1467 | 649852 | 15, 17 (Normal Colon Tissue vs. Colon Metastasis) | 6 | 0 | 6.44 | |
| 1468 | 453592 | 15, 16 (Normal Colon vs. Colon Tumor Tissue) | 19 | 6 | 3.35 | |
| 1469 | 455096 | 15, 17 (Normal Colon Tissue vs. Colon Metastasis) | 1 | 10 | | 9.32 |
| | | 16, 17 (Colon Tumor Tissue vs. Colon Metastasis) | 2 | 10 | | 4.92 |
| 1470 | 446199 | 16, 17 (Colon Tumor Tissue vs. Colon Metastasis) | 1 | 8 | | 7.88 |
| 1471 | 558427 | 15, 16 (Normal Colon vs. Colon Tumor Tissue) | 4 | 15 | | 3.55 |
| 1472 | 450255 | 16, 17 (Colon Tumor Tissue vs. Colon Metastasis) | 0 | 6 | | 5.91 |
| 1473 | 452026 | 15, 16 (Normal Colon vs. Colon Tumor Tissue) | 35 | 14 | 2.64 | |
| 1474 | 374971 | 16, 17 (Colon Tumor Tissue vs. Colon Metastasis) | 1 | 8 | | 7.88 |
| | | 08, 09 (Lung, High Met vs. Lung, Low Met) | 0 | 16 | | 11.45 |
| | | 15, 17 (Normal Colon Tissue vs. Colon Metastasis) | 0 | 8 | | 7.45 |
| 1475 | 446404 | 15, 17 (Normal Colon Tissue vs. Colon Metastasis) | 73 | 0 | 78.36 | |
| | | 18, 20 (Normal Colon Tissue vs. Colon Metastasis) | 23 | 0 | 19.66 | |
| | | 15, 16 (Normal Colon vs. Colon Tumor Tissue) | 73 | 1 | 77.16 | |
| | | 18, 19 (Normal Colon Tissue vs. Colon Tumor) | 23 | 0 | 26.28 | |
| 1476 | 549591 | 16, 17 (Colon Tumor Tissue vs. Colon Metastasis) | 7 | 24 | | 3.38 |
| | | 15, 17 (Normal Colon Tissue vs. Colon Metastasis) | 2 | 24 | | 11.18 |
| 1477 | 640135 | 15, 17 (Normal Colon Tissue vs. Colon Metastasis) | 8 | 0 | 8.59 | |
| | | 16, 17 (Colon Tumor Tissue vs. Colon Metastasis) | 6 | 0 | 6.09 | |
| 1478 | 646248 | 16, 17 (Colon Tumor Tissue vs. Colon Metastasis) | 6 | 0 | 6.09 | |
| 1479 | 639705 | 15, 17 (Normal Colon Tissue vs. Colon Metastasis) | 10 | 0 | 10.73 | |
| | | 15, 16 (Normal Colon vs. Colon Tumor Tissue) | 10 | 0 | 10.57 | |
| 1480 | 483084 | 16, 17 (Colon Tumor Tissue vs. Colon Metastasis) | 7 | 0 | 7.11 | |
| 1481 | 464029 | 16, 17 (Colon Tumor Tissue vs. Colon Metastasis) | 1 | 9 | | 8.86 |
| 1482 | 428005 | 15, 17 (Normal Colon Tissue vs. Colon Metastasis) | 0 | 8 | | 7.45 |
| | | 16, 17 (Colon Tumor Tissue vs. Colon Metastasis) | 0 | 8 | | 7.88 |
| 1483 | 91178 | 15, 17 (Normal Colon Tissue vs. Colon Metastasis) | 0 | 7 | | 6.52 |
| 1484 | 550571 | 16, 17 (Colon Tumor Tissue vs. Colon Metastasis) | 0 | 6 | | 5.91 |
| 1485 | 735028 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1486 | 559409 | 16, 17 (Colon Tumor Tissue vs. Colon Metastasis) | 0 | 6 | | 5.91 |
| 1487 | 551172 | 15, 16 (Normal Colon vs. Colon Tumor Tissue) | 1 | 10 | | 9.46 |
| 1488 | 648872 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1489 | 446404 | 18, 19 (Normal Colon Tissue vs. Colon Tumor) | 23 | 0 | 26.28 | |
| | | 18, 20 (Normal Colon Tissue vs. Colon Metastasis) | 23 | 0 | 19.66 | |
| | | 15, 16 (Normal Colon vs. Colon Tumor Tissue) | 73 | 1 | 77.16 | |
| | | 15, 17 (Normal Colon Tissue vs. Colon Metastasis) | 73 | 0 | 78.36 | |
| 1490 | 734063 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1491 | 467991 | 15, 16 (Normal Colon vs. Colon Tumor Tissue) | 7 | 0 | 7.4 | |
| 1492 | 454050 | 15, 17 (Normal Colon Tissue vs. Colon Metastasis) | 8 | 1 | 8.59 | |
| 1493 | 734646 | 15, 16 (Normal Colon vs. Colon Tumor Tissue) | 0 | 14 | | 13.25 |
| | | 16, 17 (Colon Tumor Tissue vs. Colon Metastasis) | 14 | 0 | 14.22 | |
| 1494 | 450192 | 16, 17 (Colon Tumor Tissue vs. Colon Metastasis) | 0 | 7 | | 6.89 |
| 1495 | 403978 | 23, 24 (Normal Lung Tissue vs. Lung Tumor Tissue) | 23 | 50 | | 2.15 |
| | | 15, 16 (Normal Colon vs. Colon Tumor Tissue) | 7 | 19 | | 2.57 |
| 1496 | 734209 | 16, 17 (Colon Tumor Tissue vs. Colon Metastasis) | 9 | 0 | 9.14 | |
| | | 15, 16 (Normal Colon vs. Colon Tumor Tissue) | 0 | 9 | | 8.51 |
| 1497 | 14805 | 16, 17 (Colon Tumor Tissue vs. Colon Metastasis) | 2 | 10 | | 4.92 |
| 1498 | 230995 | 15, 16 (Normal Colon vs. Colon Tumor Tissue) | 0 | 7 | | 6.62 |
| 1499 | 120049 | 15, 17 (Normal Colon Tissue vs. Colon Metastasis) | 10 | 2 | 5.37 | |
| 1500 | 642142 | 16, 17 (Colon Tumor Tissue vs. Colon Metastasis) | 11 | 0 | 11.17 | |
| | | 15, 16 (Normal Colon vs. Colon Tumor Tissue) | 2 | 11 | | 5.2 |
| 1501 | 403978 | 15, 16 (Normal Colon vs. Colon Tumor Tissue) | 7 | 19 | | 2.57 |
| | | 23, 24 (Normal Lung Tissue vs. Lung Tumor Tissue) | 23 | 50 | | 2.15 |
| 1502 | 386543 | 15, 17 (Normal Colon Tissue vs. Colon Metastasis) | 3 | 23 | | 7.14 |
| | | 16, 17 (Colon Tumor Tissue vs. Colon Metastasis) | 3 | 23 | | 7.55 |
| 1503 | 379105 | 15, 16 (Normal Colon vs. Colon Tumor Tissue) | 0 | 8 | | 7.57 |
| 1504 | 450255 | 16, 17 (Colon Tumor Tissue vs. Colon Metastasis) | 0 | 6 | | 5.91 |
| 1505 | 730143 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1506 | 734209 | 15, 16 (Normal Colon vs. Colon Tumor Tissue) | 0 | 9 | | 8.51 |
| | | 16, 17 (Colon Tumor Tissue vs. Colon Metastasis) | 9 | 0 | 9.14 | |
| 1507 | 401553 | 15, 17 (Normal Colon Tissue vs. Colon Metastasis) | 8 | 0 | 8.59 | |
| 1508 | 72979 | 18, 19 (Normal Colon Tissue vs. Colon Tumor) | 1 | 36 | | 31.5 |
| | | 19, 20 (Colon Tumor Tissue vs. Colon Metastasis) | 36 | 4 | 6.73 | |
| | | 15, 17 (Normal Colon Tissue vs. Colon Metastasis) | 46 | 311 | | 6.3 |
| | | 08, 09 (Lung, High Met vs. Lung, Low Met) | 18 | 0 | 25.15 | |
| | | 15, 16 (Normal Colon vs. Colon Tumor Tissue) | 46 | 193 | | 3.97 |
| 1509 | 726307 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1510 | 230995 | 15, 16 (Normal Colon vs. Colon Tumor Tissue) | 0 | 7 | | 6.62 |
| 1511 | 3524 | 01, 02 (Colon, High Met vs. Colon, Low Met) | 21 | 6 | 3.23 | |
| 1512 | 8112 | 03, 04 (Breast, High Met vs. Breast, Non-Met) | 8 | 0 | 7.81 | |
| 1513 | 5240 | 08, 09 (Lung, High Met vs. Lung, Low Met) | 18 | 10 | 2.52 | |
| | | 03, 04 (Breast, High Met vs. Breast, Non-Met) | 12 | 2 | 5.85 | |
| 1514 | 447326 | 15, 17 (Normal Colon Tissue vs. Colon Metastasis) | 6 | 0 | 6.44 | |
| 1515 | 2676 | 01, 02 (Colon, High Met vs. Colon, Low Met) | 2 | 17 | | 9.22 |
| 1516 | 736701 | 23, 24 (Normal Lung Tissue vs. Lung Tumor Tissue) | 10 | 0 | 10.1 | |
| | | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1517 | 736701 | 15, 16 (Normal Colon vs. Colon Tumor Tissue) | 0 | 6 | | 5.68 |
| | | 23, 24 (Normal Lung Tissue vs. Lung Tumor Tissue) | 10 | 0 | 10.1 | |
| | | 16, 17 (Colon Tumor Tissue vs. Colon Metastasis) | 6 | 0 | 6.09 | |
| 1518 | 8371 | 01, 02 (Colon, High Met vs. Colon, Low Met) | 0 | 6 | | 6.5 |
| 1520 | 185542 | 03, 04 (Breast, High Met vs. Breast, Non-Met) | 0 | 10 | | 10.25 |
| 1521 | 448046 | 15, 17 (Normal Colon Tissue vs. Colon Metastasis) | 0 | 12 | | 11.18 |
| | | 16, 17 (Colon Tumor Tissue vs. Colon Metastasis) | 3 | 12 | | 3.94 |
| 1522 | 185422 | 03, 04 (Breast, High Met vs. Breast, Non-Met) | 0 | 32 | | 32.8 |
| 1523 | 650448 | 15, 17 (Normal Colon Tissue vs. Colon Metastasis) | 7 | 0 | 7.51 | |
| 1524 | 5753 | 01, 02 (Colon, High Met vs. Colon, Low Met) | 0 | 10 | | 10.84 |
| 1526 | 1644 | 03, 04 (Breast, High Met vs. Breast, Non-Met) | 27 | 57 | | 2.16 |
| | | 01, 02 (Colon, High Met vs. Colon, Low Met) | 11 | 33 | | 3.25 |
| 1527 | 4453 | 03, 04 (Breast, High Met vs. Breast, Non-Met) | 10 | 2 | 4.88 | |
| 1528 | 454152 | 15, 16 (Normal Colon vs. Colon Tumor Tissue) | 11 | 3 | 3.88 | |
| | | 15, 17 (Normal Colon Tissue vs. Colon Metastasis) | 11 | 1 | 11.81 | |
| 1529 | 9913 | 03, 04 (Breast, High Met vs. Breast, Non-Met) | 7 | 0 | 6.83 | |
| 1530 | 1350 | 01, 02 (Colon, High Met vs. Colon, Low Met) | 3 | 44 | | 15.9 |
| 1531 | 188 | 03, 04 (Breast, High Met vs. Breast, Non-Met) | 129 | 309 | | 2.46 |
| | | 21, 22 (Normal Prostate vs. Prostate Cancer) | 71 | 166 | | 2.38 |
| 1532 | 4471 | 03, 04 (Breast, High Met vs. Breast, Non-Met) | 10 | 1 | 9.76 | |
| 1533 | 2622 | 03, 04 (Breast, High Met vs. Breast, Non-Met) | 7 | 31 | | 4.54 |
| 1534 | 185465 | 23, 24 (Normal Lung Tissue vs. Lung Tumor Tissue) | 25 | 9 | 2.81 | |
| | | 16, 17 (Colon Tumor Tissue vs. Colon Metastasis) | 4 | 14 | | 3.45 |
| | | 03, 04 (Breast, High Met vs. Breast, Non-Met) | 2 | 14 | | 7.17 |
| 1535 | 19205 | 03, 04 (Breast, High Met vs. Breast, Non-Met) | 0 | 6 | | 6.15 |
| 1536 | 185635 | 03, 04 (Breast, High Met vs. Breast, Non-Met) | 0 | 7 | | 7.17 |
| 1537 | 5289 | 03, 04 (Breast, High Met vs. Breast, Non-Met) | 19 | 2 | 9.27 | |
| 1538 | 779 | 01, 02 (Colon, High Met vs. Colon, Low Met) | 27 | 54 | | 2.17 |
| | | 03, 04 (Breast, High Met vs. Breast, Non-Met) | 60 | 22 | 2.66 | |
| 1539 | 779 | 01, 02 (Colon, High Met vs. Colon, Low Met) | 27 | 54 | | 2.17 |
| | | 03, 04 (Breast, High Met vs. Breast, Non-Met) | 60 | 22 | 2.66 | |
| 1540 | 5289 | 03, 04 (Breast, High Met vs. Breast, Non-Met) | 19 | 2 | 9.27 | |
| 1541 | 456808 | 15, 16 (Normal Colon vs. Colon Tumor Tissue) | 7 | 42 | | 5.68 |
| | | 16, 17 (Colon Tumor Tissue vs. Colon Metastasis) | 42 | 1 | 42.65 | |
| 1543 | 546642 | 16, 17 (Colon Tumor Tissue vs. Colon Metastasis) | 11 | 0 | 11.17 | |
| 1544 | 649732 | 16, 17 (Colon Tumor Tissue vs. Colon Metastasis) | 7 | 0 | 7.11 | |
| 1545 | 5240 | 03, 04 (Breast, High Met vs. Breast, Non-Met) | 12 | 2 | 5.85 | |
| | | 08, 09 (Lung, High Met vs. Lung, Low Met) | 18 | 10 | 2.52 | |
| 1546 | 448046 | 15, 17 (Normal Colon Tissue vs. Colon Metastasis) | 0 | 12 | | 11.18 |
| | | 16, 17 (Colon Tumor Tissue vs. Colon Metastasis) | 3 | 12 | | 3.94 |
| 1547 | 650476 | 15, 17 (Normal Colon Tissue vs. Colon Metastasis) | 7 | 0 | 7.51 | |
| 1548 | 379341 | 08, 09 (Lung, High Met vs. Lung, Low Met) | 2 | 21 | | 7.51 |
| | | 15, 17 (Normal Colon Tissue vs. Colon Metastasis) | 3 | 14 | | 4.35 |
| 1549 | 401849 | 16, 17 (Colon Tumor Tissue vs. Colon Metastasis) | 0 | 15 | | 14.77 |
| | | 15, 16 (Normal Colon vs. Colon Tumor Tissue) | 7 | 0 | 7.4 | |
| 1550 | 11452 | 01, 02 (Colon, High Met vs. Colon, Low Met) | 0 | 6 | | 6.5 |
| 1551 | 185417 | 08, 09 (Lung, High Met vs. Lung, Low Met) | 8 | 56 | | 5.01 |
| | | 03, 04 (Breast, High Met vs. Breast, Non-Met) | 4 | 32 | | 8.2 |
| 1552 | 4471 | 03, 04 (Breast, High Met vs. Breast, Non-Met) | 10 | 1 | 9.76 | |
| 1553 | 2557 | 01, 02 (Colon, High Met vs. Colon, Low Met) | 23 | 8 | 2.65 | |
| 1554 | 3656 | 01, 02 (Colon, High Met vs. Colon, Low Met) | 2 | 12 | | 6.5 |
| 1555 | 2327 | 03, 04 (Breast, High Met vs. Breast, Non-Met) | 8 | 19 | | 2.43 |
| | | 08, 09 (Lung, High Met vs. Lung, Low Met) | 10 | 2 | 6.99 | |
| 1556 | 449026 | 15, 17 (Normal Colon Tissue vs. Colon Metastasis) | 10 | 2 | 5.37 | |
| 1557 | 730227 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1558 | 650864 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 1559 | 530774 | 15, 17 (Normal Colon Tissue vs. Colon Metastasis) | 5 | 16 | | 2.98 |
| 1560 | 395341 | 15, 16 (Normal Colon vs. Colon Tumor Tissue) | 0 | 7 | | 6.62 |
| 1561 | 557906 | 15, 16 (Normal Colon vs. Colon Tumor Tissue) | 12 | 3 | 4.23 | |
| 1562 | 452531 | 15, 16 (Normal Colon vs. Colon Tumor Tissue) | 2 | 11 | | 5.2 |
| 1563 | 559057 | 15, 17 (Normal Colon Tissue vs. Colon Metastasis) | 0 | 7 | | 6.52 |
| 1564 | 448046 | 15, 17 (Normal Colon Tissue vs. Colon Metastasis) | 0 | 12 | | 11.18 |
| | | 16, 17 (Colon Tumor Tissue vs. Colon Metastasis) | 3 | 12 | | 3.94 |
| 1565 | 553547 | 16, 17 (Colon Tumor Tissue vs. Colon Metastasis) | 0 | 6 | | 5.91 |
| 1566 | 4636 | 03, 04 (Breast, High Met vs. Breast, Non-Met) | 3 | 15 | | 5.12 |
| 1567 | 455601 | 15, 17 (Normal Colon Tissue vs. Colon Metastasis) | 10 | 0 | 10.73 | |
| | | 16, 17 (Colon Tumor Tissue vs. Colon Metastasis) | 8 | 0 | 8.12 | |
| 1568 | 172013 | 15, 16 (Normal Colon vs. Colon Tumor Tissue) | 6 | 0 | 6.34 | |
| 1569 | 552597 | 15, 17 (Normal Colon Tissue vs. Colon Metastasis) | 0 | 7 | | 6.52 |
| 1570 | 446531 | 16, 17 (Colon Tumor Tissue vs. Colon Metastasis) | 0 | 6 | | 5.91 |
| 1571 | 639352 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1572 | 642604 | 15, 17 (Normal Colon Tissue vs. Colon Metastasis) | 10 | 0 | 10.73 | |
| | | 16, 17 (Colon Tumor Tissue vs. Colon Metastasis) | 13 | 0 | 13.2 | |
| 1573 | 558534 | 16, 17 (Colon Tumor Tissue vs. Colon Metastasis) | 0 | 7 | | 6.89 |
| 1574 | 556421 | 15, 16 (Normal Colon vs. Colon Tumor Tissue) | 12 | 1 | 12.68 | |
| 1575 | 735477 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1576 | 640703 | 16, 17 (Colon Tumor Tissue vs. Colon Metastasis) | 10 | 0 | 10.16 | |
| 1577 | 643878 | 15, 17 (Normal Colon Tissue vs. Colon Metastasis) | 8 | 0 | 8.59 | |
| 1578 | 557797 | 16, 17 (Colon Tumor Tissue vs. Colon Metastasis) | 3 | 16 | | 5.25 |
| 1579 | 557200 | 16, 17 (Colon Tumor Tissue vs. Colon Metastasis) | 1 | 10 | | 9.85 |
| | | 15, 17 (Normal Colon Tissue vs. Colon Metastasis) | 1 | 10 | | 9.32 |
| 1580 | 729531 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1581 | 734554 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1582 | 418008 | 15, 17 (Normal Colon Tissue vs. Colon Metastasis) | 0 | 7 | | 6.52 |
| 1583 | 558614 | 16, 17 (Colon Tumor Tissue vs. Colon Metastasis) | 1 | 9 | | 8.86 |
| 1584 | 452245 | 15, 17 (Normal Colon Tissue vs. Colon Metastasis) | 1 | 12 | | 11.18 |
| | | 16, 17 (Colon Tumor Tissue vs. Colon Metastasis) | 1 | 12 | | 11.82 |
| 1585 | 449891 | 15, 16 (Normal Colon vs. Colon Tumor Tissue) | 8 | 1 | 8.46 | |
| 1587 | 6162 | 03, 04 (Breast, High Met vs. Breast, Non-Met) | 13 | 0 | 12.68 | |
| 1588 | 6162 | 03, 04 (Breast, High Met vs. Breast, Non-Met) | 13 | 0 | 12.68 | |
| 1589 | 4809 | 08, 09 (Lung, High Met vs. Lung, Low Met) | 3 | 27 | | 6.44 |
| | | 01, 02 (Colon, High Met vs. Colon, Low Met) | 15 | 1 | 13.84 | |
| 1590 | 3926 | 03, 04 (Breast, High Met vs. Breast, Non-Met) | 0 | 9 | | 9.22 |
| 1591 | 185693 | 03, 04 (Breast, High Met vs. Breast, Non-Met) | 0 | 6 | | 6.15 |
| 1592 | 641683 | 15, 17 (Normal Colon Tissue vs. Colon Metastasis) | 8 | 0 | 8.59 | |
| | | 15, 16 (Normal Colon vs. Colon Tumor Tissue) | 8 | 0 | 8.46 | |
| 1593 | 11351 | 01, 02 (Colon, High Met vs. Colon, Low Met) | 0 | 6 | | 6.5 |
| 1594 | 650864 | 15, 17 (Normal Colon Tissue vs. Colon Metastasis) | 8 | 0 | 8.59 | |
| | | 15, 16 (Normal Colon vs. Colon Tumor Tissue) | 8 | 0 | 8.46 | |
| 1595 | 460445 | 16, 17 (Colon Tumor Tissue vs. Colon Metastasis) | 0 | 6 | | 5.91 |
| 1596 | 447669 | 15, 16 (Normal Colon vs. Colon Tumor Tissue) | 4 | 16 | | 3.78 |
| 1597 | 227936 | 15, 16 (Normal Colon vs. Colon Tumor Tissue) | 0 | 8 | | 7.57 |
| 1598 | 639459 | 16, 17 (Colon Tumor Tissue vs. Colon Metastasis) | 9 | 0 | 9.14 | |
| | | 15, 17 (Normal Colon Tissue vs. Colon Metastasis) | 11 | 0 | 11.81 | |
| 1599 | 650195 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1600 | 734793 | 16, 17 (Colon Tumor Tissue vs. Colon Metastasis) | 9 | 0 | 9.14 | |
| | | 15, 16 (Normal Colon vs. Colon Tumor Tissue) | 0 | 9 | | 8.51 |
| 1601 | 540787 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1602 | 400654 | 16, 17 (Colon Tumor Tissue vs. Colon Metastasis) | 1 | 8 | | 7.88 |
| 1603 | 731467 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1604 | 4045 | 03, 04 (Breast, High Met vs. Breast, Non-Met) | 10 | 1 | 9.76 | |
| | | 01, 02 (Colon, High Met vs. Colon, Low Met) | 2 | 11 | | 5.96 |
| 1605 | 447669 | 15, 16 (Normal Colon vs. Colon Tumor Tissue) | 4 | 16 | | 3.78 |
| 1606 | 11351 | 01, 02 (Colon, High Met vs. Colon, Low Met) | 0 | 6 | | 6.5 |
| 1607 | 648931 | 15, 17 (Normal Colon Tissue vs. Colon Metastasis) | 9 | 0 | 9.66 | |
| 1608 | 726786 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 1609 | 4508 | 01, 02 (Colon, High Met vs. Colon, Low Met) | 1 | 12 | | 13.01 |
| 1610 | 415058 | 16, 17 (Colon Tumor Tissue vs. Colon Metastasis) | 0 | 6 | | 5.91 |
| 1611 | 450633 | 15, 16 (Normal Colon vs. Colon Tumor Tissue) | 34 | 13 | 2.76 | |
| | | 15, 17 (Normal Colon Tissue vs. Colon Metastasis) | 34 | 7 | 5.21 | |
| 1612 | 736955 | 15, 16 (Normal Colon vs. Colon Tumor Tissue) | 0 | 13 | | 12.3 |
| | | 16, 17 (Colon Tumor Tissue vs. Colon Metastasis) | 13 | 0 | 13.2 | |
| 1613 | 729851 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1614 | 2512 | 03, 04 (Breast, High Met vs. Breast, Non-Met) | 7 | 0 | 6.83 | |
| 1615 | 452704 | 15, 16 (Normal Colon vs. Colon Tumor Tissue) | 10 | 1 | 10.57 | |
| 1616 | 4589 | 01, 02 (Colon, High Met vs. Colon, Low Met) | 14 | 2 | 6.46 | |
| 1617 | 4727 | 03, 04 (Breast, High Met vs. Breast, Non-Met) | 19 | 0 | 18.54 | |
| 1618 | 454380 | 15, 16 (Normal Colon vs. Colon Tumor Tissue) | 3 | 14 | | 4.42 |
| 1619 | 553912 | 16, 17 (Colon Tumor Tissue vs. Colon Metastasis) | 0 | 7 | | 6.89 |
| | | 15, 17 (Normal Colon Tissue vs. Colon Metastasis) | 0 | 7 | | 6.52 |
| 1620 | 450004 | 15, 16 (Normal Colon vs. Colon Tumor Tissue) | 0 | 13 | | 12.3 |
| | | 16, 17 (Colon Tumor Tissue vs. Colon Metastasis) | 13 | 2 | 6.6 | |
| 1621 | 448193 | 16, 17 (Colon Tumor Tissue vs. Colon Metastasis) | 10 | 2 | 5.08 | |
| | | 15, 16 (Normal Colon vs. Colon Tumor Tissue) | 1 | 10 | | 9.46 |
| 1622 | 549591 | 15, 17 (Normal Colon Tissue vs. Colon Metastasis) | 2 | 24 | | 11.18 |
| | | 16, 17 (Colon Tumor Tissue vs. Colon Metastasis) | 7 | 24 | | 3.38 |
| 1623 | 448511 | 15, 17 (Normal Colon Tissue vs. Colon Metastasis) | 3 | 26 | | 8.07 |
| | | 16, 17 (Colon Tumor Tissue vs. Colon Metastasis) | 4 | 26 | | 6.4 |
| 1624 | 335 | 13, 14 (bFGF Treated HMVEC vs. VEGF-Treated HMVEC) | 3 | 15 | | 4.92 |
| | | 15, 16 (Normal Colon vs. Colon Tumor Tissue) | 12 | 38 | | 3 |
| | | 15, 17 (Normal Colon Tissue vs. Colon Metastasis) | 12 | 41 | | 3.18 |
| | | 18, 19 (Normal Colon Tissue vs. Colon Tumor) | 0 | 29 | | 25.38 |
| | | 19, 20 (Colon Tumor Tissue vs. Colon Metastasis) | 29 | 4 | 5.42 | |
| | | 12, 14 (Untreated HMVEC vs. VEGF-Treated HMVEC) (Untreated | 1 | 15 | | 14.69 |
| | | HMVEC vs. VEGF-Treated HMVEC) | | | | |
| 1625 | 561382 | 15, 16 (Normal Colon vs. Colon Tumor Tissue) | 12 | 2 | 6.34 | |
| 1626 | 3447 | 08, 09 (Lung, High Met vs. Lung, Low Met) | 0 | 13 | | 9.3 |
| | | 01, 02 (Colon, High Met vs. Colon, Low Met) | 2 | 16 | | 8.67 |
| 1627 | 639896 | 16, 17 (Colon Tumor Tissue vs. Colon Metastasis) | 8 | 0 | 8.12 | |
| 1628 | 1353 | 01, 02 (Colon, High Met vs. Colon, Low Met) | 39 | 13 | 2.77 | |
| | | 03, 04 (Breast, High Met vs. Breast, Non-Met) | 37 | 17 | 2.12 | |
| 1629 | 3031 | 01, 02 (Colon, High Met vs. Colon, Low Met) | 7 | 18 | | 2.79 |
| 1630 | 557928 | 16, 17 (Colon Tumor Tissue vs. Colon Metastasis) | 0 | 6 | | 5.91 |
| 1631 | 4727 | 03, 04 (Breast, High Met vs. Breast, Non-Met) | 19 | 0 | 18.54 | |
| 1632 | 4046 | 01, 02 (Colon, High Met vs. Colon, Low Met) | 4 | 14 | | 3.79 |
| 1633 | 10882 | 03, 04 (Breast, High Met vs. Breast, Non-Met) | 6 | 20 | | 3.42 |
| 1634 | 646283 | 15, 17 (Normal Colon Tissue vs. Colon Metastasis) | 9 | 0 | 9.66 | |
| | | 15, 16 (Normal Colon vs. Colon Tumor Tissue) | 9 | 0 | 9.51 | |
| 1635 | 646283 | 15, 16 (Normal Colon vs. Colon Tumor Tissue) | 9 | 0 | 9.51 | |
| | | 15, 17 (Normal Colon Tissue vs. Colon Metastasis) | 9 | 0 | 9.66 | |
| 1636 | 139516 | 03, 04 (Breast, High Met vs. Breast, Non-Met) | 0 | 7 | | 7.17 |
| 1637 | 6184 | 01, 02 (Colon, High Met vs. Colon, Low Met) | 0 | 8 | | 8.67 |
| 1638 | 6184 | 01, 02 (Colon, High Met vs. Colon, Low Met) | 0 | 8 | | 8.67 |
| 1639 | 454653 | 16, 17 (Colon Tumor Tissue vs. Colon Metastasis) | 5 | 17 | | 3.35 |
| | | 15, 17 (Normal Colon Tissue vs. Colon Metastasis) | 3 | 17 | | 5.28 |
| 1640 | 3309 | 03, 04 (Breast, High Met vs. Breast, Non-Met) | 9 | 0 | 8.78 | |
| | | 08, 09 (Lung, High Met vs. Lung, Low Met) | 10 | 38 | | 2.72 |
| 1641 | 1037 | 03, 04 (Breast, High Met vs. Breast, Non-Met) | 4 | 22 | | 5.64 |
| 1642 | 450665 | 16, 17 (Colon Tumor Tissue vs. Colon Metastasis) | 0 | 6 | | 5.91 |
| 1643 | 726307 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1644 | 447669 | 15, 16 (Normal Colon vs. Colon Tumor Tissue) | 4 | 16 | | 3.78 |
| 1645 | 639651 | 15, 17 (Normal Colon Tissue vs. Colon Metastasis) | 6 | 0 | 6.44 | |
| 1646 | 736860 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1647 | 553705 | 15, 16 (Normal Colon vs. Colon Tumor Tissue) | 12 | 0 | 12.68 | |
| 1648 | 451375 | 15, 16 (Normal Colon vs. Colon Tumor Tissue) | 2 | 11 | | 5.2 |
| 1649 | 204862 | 15, 16 (Normal Colon vs. Colon Tumor Tissue) | 6 | 0 | 6.34 | |
| 1650 | 530883 | 15, 16 (Normal Colon vs. Colon Tumor Tissue) | 0 | 7 | | 6.62 |
| 1651 | 447539 | 15, 16 (Normal Colon vs. Colon Tumor Tissue) | 8 | 1 | 8.46 | |
| | | 15, 17 (Normal Colon Tissue vs. Colon Metastasis) | 8 | 0 | 8.59 | |
| 1652 | 455096 | 16, 17 (Colon Tumor Tissue vs. Colon Metastasis) | 2 | 10 | | 4.92 |
| | | 15, 17 (Normal Colon Tissue vs. Colon Metastasis) | 1 | 10 | | 9.32 |
| 1654 | 449142 | 16, 17 (Colon Tumor Tissue vs. Colon Metastasis) | 9 | 1 | 9.14 | |
| 1655 | 557401 | 15, 17 (Normal Colon Tissue vs. Colon Metastasis) | 10 | 2 | 5.37 | |
| 1656 | 418763 | 15, 17 (Normal Colon Tissue vs. Colon Metastasis) | 9 | 0 | 9.66 | |
| 1657 | 17649 | 03, 04 (Breast, High Met vs. Breast, Non-Met) | 6 | 0 | 5.85 | |
| 1658 | 2078 | 03, 04 (Breast, High Met vs. Breast, Non-Met) | 11 | 2 | 5.37 | |
| 1659 | 640370 | 16, 17 (Colon Tumor Tissue vs. Colon Metastasis) | 7 | 0 | 7.11 | |
| | | 15, 17 (Normal Colon Tissue vs. Colon Metastasis) | 6 | 0 | 6.44 | |
| 1660 | 449269 | 15, 17 (Normal Colon Tissue vs. Colon Metastasis) | 26 | 7 | 3.99 | |
| | | 15, 16 (Normal Colon vs. Colon Tumor Tissue) | 26 | 5 | 5.5 | |
| 1661 | 639029 | 15, 17 (Normal Colon Tissue vs. Colon Metastasis) | 6 | 0 | 6.44 | |
| 1662 | 448677 | 15, 16 (Normal Colon vs. Colon Tumor Tissue) | 11 | 1 | 11.63 | |
| 1663 | 349 | 01, 02 (Colon, High Met vs. Colon, Low Met) | 69 | 138 | | 2.17 |
| | | 03, 04 (Breast, High Met vs. Breast, Non-Met) | 77 | 1 | 75.13 | |
| | | 15, 17 (Normal Colon Tissue vs. Colon Metastasis) | 0 | 7 | | 6.52 |
| 1664 | 447494 | 15, 16 (Normal Colon vs. Colon Tumor Tissue) | 26 | 8 | 3.44 | |
| 1665 | 551433 | 16, 17 (Colon Tumor Tissue vs. Colon Metastasis) | 0 | 8 | | 7.88 |
| 1666 | 414739 | 15, 17 (Normal Colon Tissue vs. Colon Metastasis) | 2 | 14 | | 6.52 |
| | | 16, 17 (Colon Tumor Tissue vs. Colon Metastasis) | 2 | 14 | | 6.89 |
| 1667 | 640525 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1668 | 640525 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1669 | 233108 | 15, 17 (Normal Colon Tissue vs. Colon Metastasis) | 11 | 0 | 11.81 | |
| | | 15, 16 (Normal Colon vs. Colon Tumor Tissue) | 11 | 0 | 11.63 | |
| 1670 | 643594 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1671 | 1642 | 03, 04 (Breast, High Met vs. Breast, Non-Met) | 28 | 5 | 5.46 | |
| 1672 | 643804 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1673 | 449701 | 15, 16 (Normal Colon vs. Colon Tumor Tissue) | 1 | 17 | | 16.08 |
| | | 16, 17 (Colon Tumor Tissue vs. Colon Metastasis) | 17 | 1 | 17.26 | |
| 1674 | 185695 | 03, 04 (Breast, High Met vs. Breast, Non-Met) | 0 | 6 | | 6.15 |
| 1675 | 555830 | 15, 16 (Normal Colon vs. Colon Tumor Tissue) | 16 | 2 | 8.46 | |
| | | 15, 17 (Normal Colon Tissue vs. Colon Metastasis) | 16 | 2 | 8.59 | |
| 1676 | 227936 | 15, 16 (Normal Colon vs. Colon Tumor Tissue) | 0 | 8 | | 7.57 |
| 1677 | 1609 | 01, 02 (Colon, High Met vs. Colon, Low Met) | 3 | 58 | | 20.96 |
| | | 15, 17 (Normal Colon Tissue vs. Colon Metastasis) | 15 | 3 | 5.37 | |
| | | 16, 17 (Colon Tumor Tissue vs. Colon Metastasis) | 14 | 3 | 4.74 | |
| | | 03, 04 (Breast, High Met vs. Breast, Non-Met) | 10 | 2 | 4.88 | |
| 1678 | 643938 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1679 | 3656 | 01, 02 (Colon, High Met vs. Colon, Low Met) | 2 | 12 | | 6.5 |
| 1680 | 16576 | 03, 04 (Breast, High Met vs. Breast, Non-Met) | 0 | 8 | | 8.2 |
| 1681 | 9784 | 03, 04 (Breast, High Met vs. Breast, Non-Met) | 0 | 6 | | 6.15 |
| 1682 | 2557 | 01, 02 (Colon, High Met vs. Colon, Low Met) | 23 | 8 | 2.65 | |
| 1683 | 4620 | 03, 04 (Breast, High Met vs. Breast, Non-Met) | 7 | 0 | 6.83 | |
| 1684 | 43642 | 16, 17 (Colon Tumor Tissue vs. Colon Metastasis) | 0 | 6 | | 5.91 |
| 1685 | 555103 | 23, 24 (Normal Lung Tissue vs. Lung Tumor Tissue) | 0 | 6 | | 5.94 |
| | | 15, 17 (Normal Colon Tissue vs. Colon Metastasis) | 0 | 7 | | 6.52 |
| 1686 | 643341 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 1687 | 185531 | 03, 04 (Breast, High Met vs. Breast, Non-Met) | 1 | 9 | | 9.22 |
| | | 08, 09 (Lung, High Met vs. Lung, Low Met) | 0 | 13 | | 9.3 |
| 1688 | 4045 | 01, 02 (Colon, High Met vs. Colon, Low Met) | 2 | 11 | | 5.96 |
| | | 03, 04 (Breast, High Met vs. Breast, Non-Met) | 10 | 1 | 9.76 | |
| 1689 | 400258 | 16, 17 (Colon Tumor Tissue vs. Colon Metastasis) | 6 | 0 | 6.09 | |
| 1690 | 96618 | 03, 04 (Breast, High Met vs. Breast, Non-Met) | 4 | 13 | | 3.33 |
| 1691 | 646060 | 15, 17 (Normal Colon Tissue vs. Colon Metastasis) | 13 | 0 | 13.95 | |
| | | 15, 16 (Normal Colon vs. Colon Tumor Tissue) | 13 | 3 | 4.58 | |
| 1692 | 5665 | 03, 04 (Breast, High Met vs. Breast, Non-Met) | 13 | 0 | 12.68 | |
| 1693 | 149265 | 03, 04 (Breast, High Met vs. Breast, Non-Met) | 1 | 16 | | 16.4 |
| 1694 | 727314 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1695 | 736349 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1696 | 648931 | 15, 17 (Normal Colon Tissue vs. Colon Metastasis) | 9 | 0 | 9.66 | |
| 1697 | 553881 | 15, 16 (Normal Colon vs. Colon Tumor Tissue) | 15 | 4 | 3.96 | |
| 1698 | 7444 | 03, 04 (Breast, High Met vs. Breast, Non-Met) | 9 | 0 | 8.78 | |
| 1699 | 150 | 03, 04 (Breast, High Met vs. Breast, Non-Met) | 51 | 24 | 2.07 | |
| | | 08, 09 (Lung, High Met vs. Lung, Low Met) | 5 | 0 | 6.99 | |
| 1700 | 2889 | 01, 02 (Colon, High Met vs. Colon, Low Met) | 8 | 19 | | 2.57 |
| 1701 | 730670 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1702 | 560984 | 16, 17 (Colon Tumor Tissue vs. Colon Metastasis) | 0 | 6 | | 5.91 |
| 1703 | 453708 | 16, 17 (Colon Tumor Tissue vs. Colon Metastasis) | 1 | 19 | | 18.71 |
| | | 15, 17 (Normal Colon Tissue vs. Colon Metastasis) | 1 | 19 | | 17.7 |
| 1704 | 48977 | 15, 17 (Normal Colon Tissue vs. Colon Metastasis) | 0 | 8 | | 7.45 |
| | | 16, 17 (Colon Tumor Tissue vs. Colon Metastasis) | 1 | 8 | | 7.88 |
| 1707 | 97507 | 15, 16 (Normal Colon vs. Colon Tumor Tissue) | 1 | 9 | | 8.51 |
| 1708 | 735966 | 15, 16 (Normal Colon vs. Colon Tumor Tissue) | 0 | 6 | | 5.68 |
| | | 16, 1 7 (Colon Tumor Tissue vs. Colon Metastasis) | 6 | 0 | 6.09 | |
| | | 23, 24 (Normal Lung Tissue vs. Lung Tumor Tissue) | 7 | 0 | 7.07 | |
| 1709 | 35 | 15, 17 (Normal Colon Tissue vs. Colon Metastasis) | 2 | 12 | | 5.59 |
| | | 03, 04 (Breast, High Met vs. Breast, Non-Met) | 386 | 1967 | | 5.22 |
| | | 08, 09 (Lung, High Met vs. Lung, Low Met) | 868 | 11 | 110.27 | |
| | | 15, 16 (Normal Colon vs. Colon Tumor Tissue) | 2 | 14 | | 6.62 |
| 1710 | 650195 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1711 | 639705 | 15, 16 (Normal Colon vs. Colon Tumor Tissue) | 10 | 0 | 10.57 | |
| | | 15, 17 (Normal Colon Tissue vs. Colon Metastasis) | 10 | 0 | 10.73 | |
| 1712 | 185465 | 16, 17 (Colon Tumor Tissue vs. Colon Metastasis) | 4 | 14 | | 3.45 |
| | | 03, 04 (Breast, High Met vs. Breast, Non-Met) | 2 | 14 | | 7.17 |
| | | 23, 24 (Normal Lung Tissue vs. Lung Tumor Tissue) | 25 | 9 | 2.81 | |
| 1713 | 378525 | 16, 17 (Colon Tumor Tissue vs. Colon Metastasis) | 9 | 1 | 9.14 | |
| 1714 | 2889 | 01, 02 (Colon, High Met vs. Colon, Low Met) | 8 | 19 | | 2.57 |
| 1715 | 557686 | 16, 17 (Colon Tumor Tissue vs. Colon Metastasis) | 0 | 6 | | 5.91 |
| 1716 | 735786 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1717 | 455145 | 15, 17 (Normal Colon Tissue vs. Colon Metastasis) | 6 | 0 | 6.44 | |
| 1718 | 639667 | 16, 17 (Colon Tumor Tissue vs. Colon Metastasis) | 9 | 0 | 9.14 | |
| 1719 | 446913 | 18, 19 (Normal Colon Tissue vs. Colon Tumor) | 10 | 0 | 11.43 | |
| | | 15, 17 (Normal Colon Tissue vs. Colon Metastasis) | 63 | 0 | 67.62 | |
| | | 18, 20 (Normal Colon Tissue vs. Colon Metastasis) | 10 | 0 | 8.55 | |
| | | 15, 16 (Normal Colon vs. Colon Tumor Tissue) | 63 | 1 | 66.59 | |
| 1720 | 402494 | 15, 16 (Normal Colon vs. Colon Tumor Tissue) | 3 | 13 | | 4.1 |
| | | 16, 17 (Colon Tumor Tissue vs. Colon Metastasis) | 13 | 3 | 4.4 | |
| 1721 | 734256 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1722 | 734256 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1723 | 559362 | 16, 17 (Colon Tumor Tissue vs. Colon Metastasis) | 1 | 8 | | 7.88 |
| 1724 | 639651 | 15, 17 (Normal Colon Tissue vs. Colon Metastasis) | 6 | 0 | 6.44 | |
| 1725 | 419774 | 15, 17 (Normal Colon Tissue vs. Colon Metastasis) | 2 | 12 | | 5.59 |
| 1726 | 555318 | 16, 17 (Colon Tumor Tissue vs. Colon Metastasis) | 2 | 12 | | 5.91 |
| 1727 | 449956 | 16, 17 (Colon Tumor Tissue vs. Colon Metastasis) | 10 | 1 | 10.16 | |
| 1728 | 558427 | 15, 16 (Normal Colon vs. Colon Tumor Tissue) | 4 | 15 | | 3.55 |
| 1729 | 7531 | 01, 02 (Colon, High Met vs. Colon, Low Met) | 1 | 8 | | 8.67 |
| 1730 | 446514 | 16, 17 (Colon Tumor Tissue vs. Colon Metastasis) | 8 | 1 | 8.12 | |
| 1731 | 456808 | 16, 17 (Colon Tumor Tissue vs. Colon Metastasis) | 42 | 1 | 42.65 | |
| | | 15, 16 (Normal Colon vs. Colon Tumor Tissue) | 7 | 42 | | 5.68 |
| 1732 | 447035 | 16, 17 (Colon Tumor Tissue vs. Colon Metastasis) | 8 | 1 | 8.12 | |
| 1733 | 446913 | 15, 16 (Normal Colon vs. Colon Tumor Tissue) | 63 | 1 | 66.59 | |
| | | 15, 17 (Normal Colon Tissue vs. Colon Metastasis) | 63 | 0 | 67.62 | |
| | | 18, 19 (Normal Colon Tissue vs. Colon Tumor) | 10 | 0 | 11.43 | |
| | | 18, 20 (Normal Colon Tissue vs. Colon Metastasis) | 10 | 0 | 8.55 | |
| 1734 | 446900 | 16, 17 (Colon Tumor Tissue vs. Colon Metastasis) | 2 | 11 | | 5.42 |
| | | 15, 17 (Normal Colon Tissue vs. Colon Metastasis) | 1 | 11 | | 10.25 |
| 1735 | 504513 | 15, 17 (Normal Colon Tissue vs. Colon Metastasis) | 6 | 0 | 6.44 | |
| 1736 | 380477 | 15, 17 (Normal Colon Tissue vs. Colon Metastasis) | 7 | 0 | 7.51 | |
| 1738 | 8259 | 01, 02 (Colon, High Met vs. Colon, Low Met) | 10 | 0 | 9.22 | |
| | | 08, 09 (Lung, High Met vs. Lung, Low Met) | 13 | 49 | | 2.7 |
| 1739 | 8259 | 01, 02 (Colon, High Met vs. Colon, Low Met) | 10 | 0 | 9.22 | |
| | | 08, 09 (Lung, High Met vs. Lung, Low Met) | 13 | 49 | | 2.7 |
| 1740 | 552968 | 16, 17 (Colon Tumor Tissue vs. Colon Metastasis) | 0 | 9 | | 8.86 |
| | | 15, 17 (Normal Colon Tissue vs. Colon Metastasis) | 0 | 9 | | 8.38 |
| 1741 | 650845 | 16, 17 (Colon Tumor Tissue vs. Colon Metastasis) | 6 | 0 | 6.09 | |
| 1742 | 648594 | 15, 17 (Normal Colon Tissue vs. Colon Metastasis) | 10 | 0 | 10.73 | |
| 1743 | 648594 | 15, 17 (Normal Colon Tissue vs. Colon Metastasis) | 10 | 0 | 10.73 | |
| 1744 | 2796 | 03, 04 (Breast, High Met vs. Breast, Non-Met) | 8 | 37 | | 4.74 |
| 1745 | 5753 | 01, 02 (Colon, High Met vs. Colon, Low Met) | 0 | 10 | | 10.84 |
| 1746 | 734256 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1747 | 449580 | 15, 16 (Normal Colon vs. Colon Tumor Tissue) | 16 | 4 | 4.23 | |
| 1748 | 553705 | 15, 16 (Normal Colon vs. Colon Tumor Tissue) | 12 | 0 | 12.68 | |
| 1749 | 730670 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1750 | 15035 | 15, 16 (Normal Colon vs. Colon Tumor Tissue) | 8 | 0 | 8.46 | |
| | | 15, 17 (Normal Colon Tissue vs. Colon Metastasis) | 8 | 1 | 8.59 | |
| 1751 | 394436 | 15, 17 (Normal Colon Tissue vs. Colon Metastasis) | 7 | 0 | 7.51 | |
| 1752 | 726810 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1753 | 352763 | 16, 17 (Colon Tumor Tissue vs. Colon Metastasis) | 1 | 10 | | 9.85 |
| 1754 | 3506 | 03, 04 (Breast, High Met vs. Breast, Non-Met) | 1 | 10 | | 10.25 |
| 1755 | 726377 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1756 | 562111 | 16, 17 (Colon Tumor Tissue vs. Colon Metastasis) | 2 | 13 | | 6.4 |
| | | 15, 17 (Normal Colon Tissue vs. Colon Metastasis) | 2 | 13 | | 6.06 |
| 1757 | 404475 | 16, 17 (Colon Tumor Tissue vs. Colon Metastasis) | 11 | 2 | 5.59 | |
| | | 15, 17 (Normal Colon Tissue vs. Colon Metastasis) | 19 | 2 | 10.2 | |
| 1758 | 13824 | 03, 04 (Breast, High Met vs. Breast, Non-Met) | 6 | 0 | 5.85 | |
| 1759 | 558222 | 16, 17 (Colon Tumor Tissue vs. Colon Metastasis) | 0 | 6 | | 5.91 |
| 1760 | 2834 | 01, 02 (Colon, High Met vs. Colon, Low Met) | 8 | 22 | | 2.98 |
| 1761 | 453470 | 15, 16 (Normal Colon vs. Colon Tumor Tissue) | 12 | 1 | 12.68 | |
| | | 15, 17 (Normal Colon Tissue vs. Colon Metastasis) | 12 | 1 | 12.88 | |
| 1762 | 558682 | 16, 17 (Colon Tumor Tissue vs. Colon Metastasis) | 3 | 12 | | 3.94 |
| 1763 | 641710 | 15, 17 (Normal Colon Tissue vs. Colon Metastasis) | 6 | 0 | 6.44 | |
| 1764 | 640221 | 15, 17 (Normal Colon Tissue vs. Colon Metastasis) | 8 | 0 | 8.59 | |
| 1765 | 559057 | 15, 17 (Normal Colon Tissue vs. Colon Metastasis) | 0 | 7 | | 6.52 |
| 1766 | 551433 | 16, 17 (Colon Tumor Tissue vs. Colon Metastasis) | 0 | 8 | | 7.88 |
| 1767 | 5729 | 01, 02 (Colon, High Met vs. Colon, Low Met) | 1 | 10 | | 10.84 |
| 1768 | 352763 | 16, 17 (Colon Tumor Tissue vs. Colon Metastasis) | 1 | 10 | | 9.85 |
| 1769 | 375651 | 16, 17 (Colon Tumor Tissue vs. Colon Metastasis) | 8 | 0 | 8.12 | |
| 1770 | 644032 | 16, 17 (Colon Tumor Tissue vs. Colon Metastasis) | 124 | 0 | 125.92 | |
| | | 15, 17 (Normal Colon Tissue vs. Colon Metastasis) | 7 | 0 | 7.51 | |
| | | 15, 16 (Normal Colon vs. Colon Tumor Tissue) | 7 | 124 | | 16.76 |
| 1771 | 185562 | 03, 04 (Breast, High Met vs. Breast, Non-Met) | 0 | 9 | | 9.22 |
| 1772 | 736349 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1773 | 638870 | 16, 17 (Colon Tumor Tissue vs. Colon Metastasis) | 11 | 0 | 11.17 | |
| 1774 | 649719 | 15, 17 (Normal Colon Tissue vs. Colon Metastasis) | 6 | 0 | 6.44 | |
| 1775 | 62016 | 16, 17 (Colon Tumor Tissue vs. Colon Metastasis) | 0 | 6 | | 5.91 |
| 1776 | 2889 | 01, 02 (Colon, High Met vs. Colon, Low Met) | 8 | 19 | | 2.57 |
| 1777 | 647135 | 15, 17 (Normal Colon Tissue vs. Colon Metastasis) | 7 | 0 | 7.51 | |
| 1778 | 8283 | 01, 02 (Colon, High Met vs. Colon, Low Met) | 0 | 8 | | 8.67 |
| 1779 | 732121 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1780 | 532307 | 16, 17 (Colon Tumor Tissue vs. Colon Metastasis) | 0 | 7 | | 6.89 |
| 1781 | 6589 | 01, 02 (Colon, High Met vs. Colon, Low Met) | 1 | 9 | | 9.76 |
| 1782 | 554678 | 15, 16 (Normal Colon vs. Colon Tumor Tissue) | 10 | 2 | 5.28 | |
| 1783 | 450410 | 16, 17 (Colon Tumor Tissue vs. Colon Metastasis) | 0 | 7 | | 6.89 |
| 1784 | 643924 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1785 | 453719 | 16, 17 (Colon Tumor Tissue vs. Colon Metastasis) | 0 | 9 | | 8.86 |
| 1786 | 451811 | 15, 17 (Normal Colon Tissue vs. Colon Metastasis) | 10 | 2 | 5.37 | |
| 1787 | 453059 | 15, 17 (Normal Colon Tissue vs. Colon Metastasis) | 9 | 0 | 9.66 | |
| | | 15, 16 (Normal Colon vs. Colon Tumor Tissue) | 9 | 1 | 9.51 | |
| 1788 | 453457 | 16, 17 (Colon Tumor Tissue vs. Colon Metastasis) | 1 | 8 | | 7.88 |
| 1789 | 558454 | 15, 17 (Normal Colon Tissue vs. Colon Metastasis) | 0 | 7 | | 6.52 |
| 1790 | 417467 | 15, 17 (Normal Colon Tissue vs. Colon Metastasis) | 0 | 7 | | 6.52 |
| 1791 | 447850 | 16, 17 (Colon Tumor Tissue vs. Colon Metastasis) | 0 | 6 | | 5.91 |
| 1792 | 557948 | 16, 17 (Colon Tumor Tissue vs. Colon Metastasis) | 0 | 11 | | 10.83 |
| 1793 | 452685 | 15, 16 (Normal Colon vs. Colon Tumor Tissue) | 15 | 5 | 3.17 | |
| 1794 | 446964 | 15, 17 (Normal Colon Tissue vs. Colon Metastasis) | 1 | 10 | | 9.32 |
| 1795 | 550318 | 15, 16 (Normal Colon vs. Colon Tumor Tissue) | 15 | 3 | 5.28 | |
| 1796 | 407077 | 15, 17 (Normal Colon Tissue vs. Colon Metastasis) | 0 | 8 | | 7.45 |
| | | 16, 17 (Colon Tumor Tissue vs. Colon Metastasis) | 1 | 8 | | 7.88 |
| 1797 | 650864 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 1798 | 644721 | 15, 17 (Normal Colon Tissue vs. Colon Metastasis) | 6 | 0 | 6.44 | |
| 1799 | 485431 | 15, 16 (Normal Colon vs. Colon Tumor Tissue) | 0 | 6 | | 5.68 |
| 1800 | 651073 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1801 | 725811 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1802 | 645139 | 16, 17 (Colon Tumor Tissue vs. Colon Metastasis) | 9 | 0 | 9.14 | |
| 1803 | 185478 | 03, 04 (Breast, High Met vs. Breast, Non-Met) | 0 | 14 | | 14.35 |
| 1804 | 1441 | 01, 02 (Colon, High Met vs. Colon, Low Met) | 9 | 40 | | 4.82 |
| | | 03, 04 (Breast, High Met vs. Breast, Non-Met) | 38 | 16 | 2.32 | |
| 1805 | 640005 | 15, 16 (Normal Colon vs. Colon Tumor Tissue) | 20 | 6 | 3.52 | |
| | | 16, 17 (Colon Tumor Tissue vs. Colon Metastasis) | 6 | 0 | 6.09 | |
| | | 15, 17 (Normal Colon Tissue vs. Colon Metastasis) | 20 | 0 | 21.47 | |
| 1806 | 728273 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1807 | 185579 | 03, 04 (Breast, High Met vs. Breast, Non-Met) | 0 | 8 | | 8.2 |
| 1808 | 724473 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| | | 21, 22 (Normal Prostate vs. Prostate Cancer) | 5 | 16 | | 3.25 |
| 1809 | 559674 | 15, 17 (Normal Colon Tissue vs. Colon Metastasis) | 0 | 7 | | 6.52 |
| 1810 | 456026 | 15, 16 (Normal Colon vs. Colon Tumor Tissue) | 24 | 4 | 6.34 | |
| | | 15, 17 (Normal Colon Tissue vs. Colon Metastasis) | 24 | 8 | 3.22 | |
| 1811 | 549320 | 15, 17 (Normal Colon Tissue vs. Colon Metastasis) | 0 | 7 | | 6.52 |
| 1812 | 447338 | 15, 17 (Normal Colon Tissue vs. Colon Metastasis) | 7 | 0 | 7.51 | |
| 1813 | 560700 | 16, 17 (Colon Tumor Tissue vs. Colon Metastasis) | 0 | 6 | | 5.91 |
| 1814 | 3070 | 16, 17 (Colon Tumor Tissue vs. Colon Metastasis) | 0 | 6 | | 5.91 |
| 1815 | 3070 | 16, 17 (Colon Tumor Tissue vs. Colon Metastasis) | 0 | 6 | | 5.91 |
| 1816 | 380477 | 15, 17 (Normal Colon Tissue vs. Colon Metastasis) | 7 | 0 | 7.51 | |
| 1817 | 735040 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1818 | 378525 | 16, 17 (Colon Tumor Tissue vs. Colon Metastasis) | 9 | 1 | 9.14 | |
| 1819 | 284586 | 15, 17 (Normal Colon Tissue vs. Colon Metastasis) | 6 | 0 | 6.44 | |
| 1820 | 640276 | 16, 17 (Colon Tumor Tissue vs. Colon Metastasis) | 8 | 0 | 8.12 | |
| 1821 | 3344 | 01, 02 (Colon, High Met vs. Colon, Low Met) | 7 | 17 | | 2.63 |
| 1822 | 555830 | 15, 16 (Normal Colon vs. Colon Tumor Tissue) | 16 | 2 | 8.46 | |
| | | 15, 17 (Normal Colon Tissue vs. Colon Metastasis) | 16 | 2 | 8.59 | |
| 1823 | 726307 | l5, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1824 | 416 | 15, 16 (Normal Colon vs. Colon Tumor Tissue) | 8 | 23 | | 2.72 |
| | | 21, 22 (Normal Prostate vs. Prostate Cancer) | 11 | 31 | | 2.87 |
| | | 15, 17 (Normal Colon Tissue vs. Colon Metastasis) | 8 | 22 | | 2.56 |
| 1825 | 2543 | 01, 02 (Colon, High Met vs. Colon, Low Met) | 23 | 7 | 3.03 | |
| 1826 | 639352 | 15, 17 (Normal Colon Tissue vs. Colon Metastasis) | 7 | 0 | 7.51 | |
| | | 15, 16 (Normal Colon vs. Colon Tumor Tissue) | 7 | 0 | 7.4 | |
| 1827 | 453592 | 15, 16 (Normal Colon vs. Colon Tumor Tissue) | 19 | 6 | 3.35 | |
| 1828 | 450633 | 15, 16 (Normal Colon vs. Colon Tumor Tissue) | 34 | 13 | 2.76 | |
| | | 15, 17 (Normal Colon Tissue vs. Colon Metastasis) | 34 | 7 | 5.21 | |
| 1829 | 448383 | 15, 17 (Normal Colon Tissue vs. Colon Metastasis) | 13 | 1 | 13.95 | |
| 1830 | 648719 | 15, 17 (Normal Colon Tissue vs. Colon Metastasis) | 9 | 0 | 9.66 | |
| | | 15, 16 (Normal Colon vs. Colon Tumor Tissue) | 9 | 1 | 9.51 | |
| 1831 | 730655 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1832 | 141185 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 1833 | 640498 | 16, 17 (Colon Tumor Tissue vs. Colon Metastasis) | 6 | 0 | 6.09 | |
| 1834 | 9029 | 01, 02 (Colon, High Met vs. Colon, Low Met) | 7 | 0 | 6.46 | |
| 1835 | 559674 | 15, 17 (Normal Colon Tissue vs. Colon Metastasis) | 0 | 7 | | 6.52 |
| 1836 | 555734 | 15, 17 (Normal Colon Tissue vs. Colon Metastasis) | 0 | 10 | | 9.32 |
| | | 16, 17 (Colon Tumor Tissue vs. Colon Metastasis) | 0 | 10 | | 9.85 |
| 1837 | 1943 | 03, 04 (Breast, High Met vs. Breast, Non-Met) | 16 | 4 | 3.9 | |
| | | 01, 02 (Colon, High Met vs. Colon, Low Met) | 9 | 29 | | 3.49 |
| | | 15, 16 (Normal Colon vs. Colon Tumor Tissue) | 6 | 19 | | 3 |
| 1838 | 648320 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1839 | 558098 | 15, 16 (Normal Colon vs. Colon Tumor Tissue) | 6 | 0 | 6.34 | |
| 1840 | 468672 | 16, 17 (Colon Tumor Tissue vs. Colon Metastasis) | 0 | 8 | | 7.88 |
| | | 15, 16 (Normal Colon vs. Colon Tumor Tissue) | 6 | 0 | 6.34 | |
| 1841 | 456596 | 16, 17 (Colon Tumor Tissue vs. Colon Metastasis) | 1 | 12 | | 11.82 |
| | | 15, 17 (Normal Colon Tissue vs. Colon Metastasis) | 2 | 12 | | 5.59 |
| 1842 | 649722 | 16, 17 (Colon Tumor Tissue vs. Colon Metastasis) | 6 | 0 | 6.09 | |
| | | 15, 17 (Normal Colon Tissue vs. Colon Metastasis) | 8 | 0 | 8.59 | |
| 1843 | 550708 | 16, 17 (Colon Tumor Tissue vs. Colon Metastasis) | 1 | 8 | | 7.88 |
| 1844 | 643931 | 15, 17 (Normal Colon Tissue vs. Colon Metastasis) | 7 | 0 | 7.51 | |
| 1845 | 726927 | 16, 17 (Colon Tumor Tissue vs. Colon Metastasis) | 11 | 0 | 11.17 | |
| | | 15, 16 (Normal Colon vs. Colon Tumor Tissue) | 0 | 11 | | 10.41 |
| 1846 | 459012 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1847 | 397773 | 15, 17 (Normal Colon Tissue vs. Colon Metastasis) | 12 | 0 | 12.88 | |
| | | 16, 17 (Colon Tumor Tissue vs. Colon Metastasis) | 9 | 0 | 9.14 | |
| 1848 | 450004 | 15, 16 (Normal Colon vs. Colon Tumor Tissue) | 0 | 13 | | 12.3 |
| | | 16, 17 (Colon Tumor Tissue vs. Colon Metastasis) | 13 | 2 | 6.6 | |
| 1849 | 649732 | 16, 17 (Colon Tumor Tissue vs. Colon Metastasis) | 7 | 0 | 7.11 | |
| 1850 | 553955 | 16, 17 (Colon Tumor Tissue vs. Colon Metastasis) | 0 | 6 | | 5.91 |
| 1851 | 646309 | 16, 17 (Colon Tumor Tissue vs. Colon Metastasis) | 6 | 0 | 6.09 | |
| 1852 | 402727 | 15, 17 (Normal Colon Tissue vs. Colon Metastasis) | 5 | 17 | | 3.17 |
| 1853 | 468736 | 16, 17 (Colon Tumor Tissue vs. Colon Metastasis) | 0 | 6 | | 5.91 |
| 1854 | 650422 | 15, 16 (Normal Colon vs. Colon Tumor Tissue) | 18 | 6 | 3.17 | |
| | | 16, 17 (Colon Tumor Tissue vs. Colon Metastasis) | 6 | 0 | 6.09 | |
| | | 15, 17 (Normal Colon Tissue vs. Colon Metastasis) | 18 | 0 | 19.32 | |
| 1855 | 730533 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1856 | 726307 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1857 | 450311 | 15, 16 (Normal Colon vs. Colon Tumor Tissue) | 8 | 1 | 8.46 | |
| 1858 | 450940 | 15, 17 (Normal Colon Tissue vs. Colon Metastasis) | 1 | 9 | | 8.38 |
| | | 16, 17 (Colon Tumor Tissue vs. Colon Metastasis) | 0 | 9 | | 8.86 |
| 1859 | 726786 | 15, 16 (Normal Colon vs. Colon Tumor Tissue) | 0 | 8 | | 7.57 |
| | | 16, 17 (Colon Tumor Tissue vs. Colon Metastasis) | 8 | 0 | 8.12 | |
| 1860 | 7634 | 03, 04 (Breast, High Met vs. Breast, Non-Met) | 6 | 0 | 5.85 | |
| 1861 | 230995 | 15, 16 (Normal Colon vs. Colon Tumor Tissue) | 0 | 7 | | 6.62 |
| 1862 | 374770 | 15, 17 (Normal Colon Tissue vs. Colon Metastasis) | 8 | 0 | 8.59 | |
| | | l5, 16 (Normal Colon vs. Colon Tumor Tissue) | 8 | 0 | 8.46 | |
| 1863 | 9275 | 03, 04 (Breast, High Met vs. Breast, Non-Met) | 0 | 6 | | 6.15 |
| 1864 | 553860 | 15, 17 (Normal Colon Tissue vs. Colon Metastasis) | 0 | 9 | | 8.38 |
| 1865 | 452010 | 16, 17 (Colon Tumor Tissue vs. Colon Metastasis) | 13 | 3 | 4.4 | |
| | | 15, 16 (Normal Colon vs. Colon Tumor Tissue) | 3 | 13 | | 4.1 |
| 1866 | 649560 | 16, 17 (Colon Tumor Tissue vs. Colon Metastasis) | 7 | 0 | 7.11 | |
| 1867 | 452704 | 15, 16 (Normal Colon vs. Colon Tumor Tissue) | 10 | 1 | 10.57 | |
| 1868 | 447594 | 18, 19 (Normal Colon Tissue vs. Colon Tumor) | 11 | 2 | 6.29 | |
| | | 15, 17 (Normal Colon Tissue vs. Colon Metastasis) | 6 | 20 | | 3.11 |
| 1869 | 555444 | 16, 17 (Colon Tumor Tissue vs. Colon Metastasis) | 22 | 4 | 5.59 | |
| | | 15, 16 (Normal Colon vs. Colon Tumor Tissue) | 2 | 22 | | 10.41 |
| 1870 | 736556 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 1871 | 5289 | 03, 04 (Breast, High Met vs. Breast, Non-Met) | 19 | 2 | 9.27 | |
| 1872 | 732121 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1873 | 452567 | 16, 17 (Colon Tumor Tissue vs. Colon Metastasis) | 3 | 14 | | 4.6 |
| | | 15, 16 (Normal Colon vs. Colon Tumor Tissue) | 21 | 3 | 7.4 | |
| 1874 | 551634 | 16, 17 (Colon Tumor Tissue vs. Colon Metastasis) | 0 | 6 | | 5.91 |
| 1875 | 644099 | 15, 17 (Normal Colon Tissue vs. Colon Metastasis) | 6 | 0 | 6.44 | |
| 1876 | 726788 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1877 | 638802 | 15, 17 (Normal Colon Tissue vs. Colon Metastasis) | 6 | 0 | 6.44 | |
| 1878 | 646283 | 15, 17 (Normal Colon Tissue vs. Colon Metastasis) | 9 | 0 | 9.66 | |
| | | 15, 16 (Normal Colon vs. Colon Tumor Tissue) | 9 | 0 | 9.51 | |
| 1879 | 8403 | 03, 04 (Breast, High Met vs. Breast, Non-Met) | 6 | 0 | 5.85 | |
| 1880 | 2224 | 03, 04 (Breast, High Met vs. Breast, Non-Met) | 44 | 8 | 5.37 | |
| 1881 | 650053 | 15, 17 (Normal Colon Tissue vs. Colon Metastasis) | 10 | 0 | 10.73 | |
| | | 15, 16 (Normal Colon vs. Colon Tumor Tissue) | 10 | 1 | 10.57 | |
| 1882 | 380477 | 15, 17 (Normal Colon Tissue vs. Colon Metastasis) | 7 | 0 | 7.51 | |
| 1883 | 450867 | 15, 16 (Normal Colon vs. Colon Tumor Tissue) | 7 | 0 | 7.4 | |
| 1884 | 456764 | 16, 17 (Colon Tumor Tissue vs. Colon Metastasis) | 10 | 1 | 10.16 | |
| | | 15, 17 (Normal Colon Tissue vs. Colon Metastasis) | 14 | 1 | 15.03 | |
| 1885 | 641373 | 15, 16 (Normal Colon vs. Colon Tumor Tissue) | 15 | 3 | 5.28 | |
| | | 15, 17 (Normal Colon Tissue vs. Colon Metastasis) | 15 | 0 | 16.1 | |
| 1886 | 555882 | 16, 17 (Colon Tumor Tissue vs. Colon Metastasis) | 0 | 9 | | 8.86 |
| 1887 | 644046 | 15, 17 (Normal Colon Tissue vs. Colon Metastasis) | 9 | 0 | 9.66 | |
| 1888 | 447250 | 16, 17 (Colon Tumor Tissue vs. Colon Metastasis) | 6 | 0 | 6.09 | |
| 1889 | 456596 | 15, 17 (Normal Colon Tissue vs. Colon Metastasis) | 2 | 12 | | 5.59 |
| | | 16, 17 (Colon Tumor Tissue vs. Colon Metastasis) | 1 | 12 | | 11.82 |
| 1890 | 2218 | 01, 02 (Colon, High Met vs. Colon, Low Met) | 2 | 21 | | 11.38 |
| | | 03, 04 (Breast, High Met vs. Breast, Non-Met) | 27 | 8 | 3.29 | |
| | | 08, 09 (Lung, High Met vs. Lung, Low Met) | 5 | 0 | 6.99 | |
| 1891 | 446450 | 15, 16 (Normal Colon vs. Colon Tumor Tissue) | 11 | 3 | 3.88 | |
| 1892 | 640889 | 15, 17 (Normal Colon Tissue vs. Colon Metastasis) | 6 | 0 | 6.44 | |
| 1893 | 530774 | 15, 17 (Normal Colon Tissue vs. Colon Metastasis) | 5 | 16 | | 2.98 |
| 1894 | 649062 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1895 | 12808 | 03, 04 (Breast, High Met vs. Breast, Non-Met) | 6 | 0 | 5.85 | |
| 1896 | 468672 | 15, 16 (Normal Colon vs. Colon Tumor Tissue) | 6 | 0 | 6.34 | |
| | | 16, 17 (Colon Tumor Tissue vs. Colon Metastasis) | 0 | 8 | | 7.88 |
| 1897 | 650773 | 15, 17 (Normal Colon Tissue vs. Colon Metastasis) | 6 | 0 | 6.44 | |
| 1898 | 732237 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1899 | 650773 | 15, 17 (Normal Colon Tissue vs. Colon Metastasis) | 6 | 0 | 6.44 | |
| 1900 | 550216 | 15, 17 (Normal Colon Tissue vs. Colon Metastasis) | 0 | 7 | | 6.52 |
| 1901 | 639189 | 15, 17 (Normal Colon Tissue vs. Colon Metastasis) | 7 | 0 | 7.51 | |
| 1902 | 3447 | 01, 02 (Colon, High Met vs. Colon, Low Met) | 2 | 16 | | 8.67 |
| | | 08, 09 (Lung, High Met vs. Lung, Low Met) | 0 | 13 | | 9.3 |
| 1903 | 2012 | 03, 04 (Breast, High Met vs. Breast, Non-Met) | 20 | 58 | | 2.97 |
| | | 01, 02 (Colon, High Met vs. Colon, Low Met) | 13 | 29 | | 2.42 |
| 1904 | 642876 | 15, 17 (Normal Colon Tissue vs. Colon Metastasis) | 10 | 0 | 10.73 | |
| | | 15, 16 (Normal Colon vs. Colon Tumor Tissue) | 10 | 2 | 5.28 | |
| 1905 | 449690 | 16, 17 (Colon Tumor Tissue vs. Colon Metastasis) | 3 | 17 | | 5.58 |
| 1906 | 451208 | 15, 17 (Normal Colon Tissue vs. Colon Metastasis) | 14 | 3 | 5.01 | |
| 1907 | 725811 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1908 | 1256 | 08, 09 (Lung, High Met vs. Lung, Low Met) | 35 | 110 | | 2.25 |
| | | 03, 04 (Breast, High Met vs. Breast, Non-Met) | 14 | 31 | | 2.27 |
| 1909 | 446599 | 15, 16 (Normal Colon vs. Colon Tumor Tissue) | 13 | 2 | 6.87 | |
| 1910 | 446537 | 15, 16 (Normal Colon vs. Colon Tumor Tissue) | 9 | 1 | 9.51 | |
| 1911 | 726281 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 1912 | 11286 | 01, 02 (Colon, High Met vs. Colon, Low Met) | 0 | 6 | | 6.5 |
| 1913 | 556082 | 16, 17 (Colon Tumor Tissue vs. Colon Metastasis) | 0 | 10 | | 9.85 |
| 1914 | 97507 | 15, 16 (Normal Colon vs. Colon Tumor Tissue) | 1 | 9 | | 8.51 |
| 1915 | 535955 | 16, 17 (Colon Tumor Tissue vs. Colon Metastasis) | 0 | 10 | | 9.85 |
| | | 15, 17 (Normal Colon Tissue vs. Colon Metastasis) | 1 | 10 | | 9.32 |
| 1916 | 728251 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 1917 | 733849 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 1918 | 447574 | 15, 17 (Normal Colon Tissue vs. Colon Metastasis) | 7 | 0 | 7.51 | |
| 1919 | 7607 | 15, 17 (Normal Colon Tissue vs. Colon Metastasis) | 13 | 3 | 4.65 | |
| | | 15, 16 (Normal Colon vs. Colon Tumor Tissue) | 13 | 4 | 3.44 | |
| | | 01, 02 (Colon, High Met vs. Colon, Low Met) | 0 | 7 | | 7.59 |
| 1920 | 644032 | 15, 16 (Normal Colon vs. Colon Tumor Tissue) | 7 | 124 | | 16.76 |
| | | 16, 17 (Colon Tumor Tissue vs. Colon Metastasis) | 124 | 0 | 125.92 | |
| | | 15, 17 (Normal Colon Tissue vs. Colon Metastasis) | 7 | 0 | 7.51 | |
| 1921 | 454087 | 15, 16 (Normal Colon vs. Colon Tumor Tissue) | 14 | 3 | 4.93 | |
| | | 15, 17 (Normal Colon Tissue vs. Colon Metastasis) | 14 | 1 | 15.03 | |
| 1922 | 412364 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1923 | 535208 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1924 | 644609 | 15, 17 (Normal Colon Tissue vs. Colon Metastasis) | 7 | 0 | 7.51 | |
| 1925 | 645073 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1926 | 417467 | 15, 17 (Normal Colon Tissue vs. Colon Metastasis) | 0 | 7 | | 6.52 |
| 1927 | 554188 | 15, 16 (Normal Colon vs. Colon Tumor Tissue) | 6 | 0 | 6.34 | |
| 1928 | 647185 | 15, 17 (Normal Colon Tissue vs. Colon Metastasis) | 6 | 0 | 6.44 | |
| 1929 | 736679 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1930 | 553547 | 16, 17 (Colon Tumor Tissue vs. Colon Metastasis) | 0 | 6 | | 5.91 |
| 1931 | 641524 | 16, 17 (Colon Tumor Tissue vs. Colon Metastasis) | 6 | 0 | 6.09 | |
| 1932 | 649717 | 16, 17 (Colon Tumor Tissue vs. Colon Metastasis) | 6 | 0 | 6.09 | |
| 1933 | 451041 | 16, 17 (Colon Tumor Tissue vs. Colon Metastasis) | 0 | 6 | | 5.91 |
| 1934 | 3483 | 01, 02 (Colon, High Met vs. Colon, Low Met) | 3 | 20 | | 7.23 |
| 1935 | 500959 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 1936 | 500959 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 1937 | 697 | 03, 04 (Breast, High Met vs. Breast, Non-Met) | 30 | 72 | | 2.46 |
| | | 21, 22 (Normal Prostate vs. Prostate Cancer) | 10 | 2 | 4.92 | |
| 1938 | 736955 | 16, 17 (Colon Tumor Tissue vs. Colon Metastasis) | 13 | 0 | 13.2 | |
| | | 15, 16 (Normal Colon vs. Colon Tumor Tissue) | 0 | 13 | | 12.3 |
| 1939 | 554742 | 16, 17 (Colon Tumor Tissue vs. Colon Metastasis) | 0 | 6 | | 5.91 |
| 1940 | 642973 | 16, 17 (Colon Tumor Tissue vs. Colon Metastasis) | 7 | 0 | 7.11 | |
| 1941 | 449437 | 15, 16 (Normal Colon vs. Colon Tumor Tissue) | 14 | 3 | 4.93 | |
| | | 16, 17 (Colon Tumor Tissue vs. Colon Metastasis) | 3 | 12 | | 3.94 |
| 1942 | 467991 | 15, 16 (Normal Colon vs. Colon Tumor Tissue) | 7 | 0 | 7.4 | |
| 1943 | 650204 | 15, 17 (Normal Colon Tissue vs. Colon Metastasis) | 11 | 0 | 11.81 | |
| 1944 | 640618 | 15, 17 (Normal Colon Tissue vs. Colon Metastasis) | 6 | 0 | 6.44 | |
| 1945 | 452366 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1946 | 640276 | 16, 17 (Colon Tumor Tissue vs. Colon Metastasis) | 8 | 0 | 8.12 | |
| 1947 | 554101 | 15, 16 (Normal Colon vs. Colon Tumor Tissue) | 0 | 6 | | 5.68 |
| 1948 | 185432 | 03, 04 (Breast, High Met vs. Breast, Non-Met) | 0 | 26 | | 26.65 |
| 1949 | 455598 | 16, 17 (Colon Tumor Tissue vs. Colon Metastasis) | 0 | 6 | | 5.91 |
| 1950 | 649354 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1951 | 4408 | 03, 04 (Breast, High Met vs. Breast, Non-Met) | 4 | 17 | | 4.36 |
| | | 08, 09 (Lung, High Met vs. Lung, Low Met) | 21 | 3 | 9.78 | |
| 1952 | 452366 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1953 | 452366 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 1954 | 727331 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1955 | 644853 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 1956 | 554079 | 16, 17 (Colon Tumor Tissue vs. Colon Metastasis) | 2 | 10 | | 4.92 |
| 1957 | 556245 | 15, 16 (Normal Colon vs. Colon Tumor Tissue) | 0 | 6 | | 5.68 |
| 1958 | 557388 | 16, 17 (Colon Tumor Tissue vs. Colon Metastasis) | 13 | 2 | 6.6 | |
| | | 15, 16 (Normal Colon vs. Colon Tumor Tissue) | 0 | 13 | | 12.3 |
| 1959 | 449468 | 15, 17 (Normal Colon Tissue vs. Colon Metastasis) | 2 | 11 | | 5.12 |
| 1960 | 556245 | 15, 16 (Normal Colon vs. Colon Tumor Tissue) | 0 | 6 | | 5.68 |
| 1961 | 455327 | 15, 17 (Normal Colon Tissue vs. Colon Metastasis) | 1 | 12 | | 11.18 |
| | | 15, 16 (Normal Colon vs. Colon Tumor Tissue) | 1 | 10 | | 9.46 |
| 1962 | 546632 | 23, 24 (Normal Lung Tissue vs. Lung Tumor Tissue) | 3 | 34 | | 11.22 |
| | | 16, 17 (Colon Tumor Tissue vs. Colon Metastasis) | 15 | 0 | 15.23 | |
| 1963 | 558762 | 16, 17 (Colon Tumor Tissue vs. Colon Metastasis) | 0 | 7 | | 6.89 |
| 1964 | 550818 | 16, 17 (Colon Tumor Tissue vs. Colon Metastasis) | 1 | 8 | | 7.88 |
| 1965 | 554079 | 16, 17 (Colon Tumor Tissue vs. Colon Metastasis) | 2 | 10 | | 4.92 |
| 1966 | 452430 | 15, 17 (Normal Colon Tissue vs. Colon Metastasis) | 6 | 0 | 6.44 | |
| 1967 | 452430 | 15, 17 (Normal Colon Tissue vs. Colon Metastasis) | 6 | 0 | 6.44 | |
| 1968 | 556082 | 16, 17 (Colon Tumor Tissue vs. Colon Metastasis) | 0 | 10 | | 9.85 |
| 1969 | 514418 | 16, 17 (Colon Tumor Tissue vs. Colon Metastasis) | 0 | 9 | | 8.86 |
| | | 15, 17 (Normal Colon Tissue vs. Colon Metastasis) | 1 | 9 | | 8.38 |
| 1970 | 426895 | 15, 17 (Normal Colon Tissue vs. Colon Metastasis) | 2 | 18 | | 8.38 |
| 1971 | 560803 | 16, 17 (Colon Tumor Tissue vs. Colon Metastasis) | 0 | 7 | | 6.89 |
| | | 15, 17 (Normal Colon Tissue vs. Colon Metastasis) | 0 | 7 | | 6.52 |
| 1972 | 447737 | 15, 16 (Normal Colon vs. Colon Tumor Tissue) | 19 | 6 | 3.35 | |
| 1973 | 373432 | 15, 17 (Normal Colon Tissue vs. Colon Metastasis) | 23 | 78 | | 3.16 |
| | | 15, 16 (Normal Colon vs. Colon Tumor Tissue) | 23 | 53 | | 2.18 |
| | | 08, 09 (Lung, High Met vs. Lung, Low Met) | 3 | 49 | | 11.69 |
| 1974 | 779 | 03, 04 (Breast, High Met vs. Breast, Non-Met) | 60 | 22 | 2.66 | |
| | | 01, 02 (Colon, High Met vs. Colon, Low Met) | 27 | 54 | | 2.17 |
| 1975 | 455327 | 15, 16 (Normal Colon vs. Colon Tumor Tissue) | 1 | 10 | | 9.46 |
| | | 15, 17 (Normal Colon Tissue vs. Colon Metastasis) | 1 | 12 | | 11.18 |
| 1976 | 554742 | 16, 17 (Colon Tumor Tissue vs. Colon Metastasis) | 0 | 6 | | 5.91 |
| 1977 | 455327 | 15, 16 (Normal Colon vs. Colon Tumor Tissue) | 1 | 10 | | 9.46 |
| | | 15, 17 (Normal Colon Tissue vs. Colon Metastasis) | 1 | 12 | | 11.18 |
| 1978 | 11043 | 01, 02 (Colon, High Met vs. Colon, Low Met) | 0 | 6 | | 6.5 |
| 1979 | 727447 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 1980 | 552905 | 15, 17 (Normal Colon Tissue vs. Colon Metastasis) | 0 | 7 | | 6.52 |
| 1981 | 446900 | 16, 17 (Colon Tumor Tissue vs. Colon Metastasis) | 2 | 11 | | 5.42 |
| | | 15, 17 (Normal Colon Tissue vs. Colon Metastasis) | 1 | 11 | | 10.25 |
| 1982 | 644190 | 16, 17 (Colon Tumor Tissue vs. Colon Metastasis) | 6 | 0 | 6.09 | |
| 1983 | 455327 | 15, 16 (Normal Colon vs. Colon Tumor Tissue) | 1 | 10 | | 9.46 |
| | | 15, 17 (Normal Colon Tissue vs. Colon Metastasis) | 1 | 12 | | 11.18 |
| 1984 | 422375 | 15, 17 (Normal Colon Tissue vs. Colon Metastasis) | 2 | 11 | | 5.12 |
| 1985 | 422375 | 15, 17 (Normal Colon Tissue vs. Colon Metastasis) | 2 | 11 | | 5.12 |
| 1986 | 530774 | 15, 17 (Normal Colon Tissue vs. Colon Metastasis) | 5 | 16 | | 2.98 |
| 1987 | 554101 | 15, 16 (Normal Colon vs. Colon Tumor Tissue) | 0 | 6 | | 5.68 |
| 1988 | 5268 | 01, 02 (Colon, High Met vs. Colon, Low Met) | 0 | 15 | | 16.26 |
| 1989 | 642461 | 15, 17 (Normal Colon Tissue vs. Colon Metastasis) | 14 | 0 | 15.03 | |
| | | 16, 17 (Colon Tumor Tissue vs. Colon Metastasis) | 10 | 0 | 10.16 | |
| 1990 | 770 | 03, 04 (Breast, High Met vs. Breast, Non-Met) | 47 | 9 | 5.1 | |
| | | 15, 17 (Normal Colon Tissue vs. Colon Metastasis) | 11 | 1 | 11.81 | |
| 1991 | 3837 | 03, 04 (Breast, High Met vs. Breast, Non-Met) | 9 | 1 | 8.78 | |
| 1992 | 561382 | 15, 16 (Normal Colon vs. Colon Tumor Tissue) | 12 | 2 | 6.34 | |
| 1993 | 4408 | 08, 09 (Lung, High Met vs. Lung, Low Met) | 21 | 3 | 9.78 | |
| | | 03, 04 (Breast, High Met vs. Breast, Non-Met) | 4 | 17 | | 4.36 |
| 1994 | 5686 | 03, 04 (Breast, High Met vs. Breast, Non-Met) | 16 | 3 | 5.2 | |
| 1995 | 374609 | 21, 22 (Normal Prostate vs. Prostate Cancer) | 1 | 9 | | 9.15 |
| | | 15, 16 (Normal Colon vs. Colon Tumor Tissue) | 0 | 7 | | 6.62 |
| 1996 | 734793 | 16, 17 (Colon Tumor Tissue vs. Colon Metastasis) | 9 | 0 | 9.14 | |
| | | 15, 16 (Normal Colon vs. Colon Tumor Tissue) | 0 | 9 | | 8.51 |
| 1997 | 452430 | 15, 17 (Normal Colon Tissue vs. Colon Metastasis) | 6 | 0 | 6.44 | |
| 1998 | 450940 | 16, 17 (Colon Tumor Tissue vs. Colon Metastasis) | 0 | 9 | | 8.86 |
| | | 15, 17 (Normal Colon Tissue vs. Colon Metastasis) | 1 | 9 | | 8.38 |
| 1999 | 460445 | 16, 17 (Colon Tumor Tissue vs. Colon Metastasis) | 0 | 6 | | 5.91 |
| 2000 | 549041 | 16, 17 (Colon Tumor Tissue vs. Colon Metastasis) | 0 | 6 | | 5.91 |
| 2001 | 555276 | 16, 17 (Colon Tumor Tissue vs. Colon Metastasis) | 0 | 6 | | 5.91 |
| 2002 | 426895 | 15, 17 (Normal Colon Tissue vs. Colon Metastasis) | 2 | 18 | | 8.38 |
| 2003 | 1833 | 01, 02 (Colon, High Met vs. Colon, Low Met) | 9 | 25 | | 3.01 |
| 2004 | 446450 | 15, 16 (Normal Colon vs. Colon Tumor Tissue) | 11 | 3 | 3.88 | |
| 2005 | 650517 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 2006 | 554785 | 16, 17 (Colon Tumor Tissue vs. Colon Metastasis) | 0 | 8 | | 7.88 |
| | | 15, 17 (Normal Colon Tissue vs. Colon Metastasis) | 0 | 8 | | 7.45 |
| 2007 | 607430 | 15, 16 (Normal Colon vs. Colon Tumor Tissue) | 6 | 0 | 6.34 | |
| 2008 | 446673 | 16, 17 (Colon Tumor Tissue vs. Colon Metastasis) | 8 | 1 | 8.12 | |
| 2009 | 734685 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2010 | 11630 | 21, 22 (Normal Prostate vs. Prostate Cancer) | 1 | 12 | | 12.2 |
| | | 03, 04 (Breast, High Met vs. Breast, Non-Met) | 3 | 13 | | 4.44 |
| 2011 | 2930 | 01, 02 (Colon, High Met vs. Colon, Low Met) | 3 | 14 | | 5.06 |
| 2012 | 44424 | 16, 17 (Colon Tumor Tissue vs. Colon Metastasis) | 41 | 90 | | 2.16 |
| | | 15, 17 (Normal Colon Tissue vs. Colon Metastasis) | 25 | 90 | | 3.35 |
| | | 08, 09 (Lung, High Met vs. Lung, Low Met) | 4 | 40 | | 7.16 |
| 2013 | 452052 | 15, 17 (Normal Colon Tissue vs. Colon Metastasis) | 8 | 1 | 8.59 | |
| 2014 | 449356 | 15, 17 (Normal Colon Tissue vs. Colon Metastasis) | 7 | 21 | | 2.79 |
| | | 16, 17 (Colon Tumor Tissue vs. Colon Metastasis) | 4 | 21 | | 5.17 |
| 2015 | 726225 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 2016 | 453708 | 16, 17 (Colon Tumor Tissue vs. Colon Metastasis) | 1 | 19 | | 18.71 |
| | | 15, 17 (Normal Colon Tissue vs. Colon Metastasis) | 1 | 19 | | 17.7 |
| 2017 | 447858 | 15, 16 (Normal Colon vs. Colon Tumor Tissue) | 12 | 3 | 4.23 | |
| 2018 | 451613 | 15, 17 (Normal Colon Tissue vs. Colon Metastasis) | 4 | 14 | | 3.26 |
| 2019 | 650337 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 2020 | 62016 | 16, 17 (Colon Tumor Tissue vs. Colon Metastasis) | 0 | 6 | | 5.91 |
| 2021 | 447250 | 16, 17 (Colon Tumor Tissue vs. Colon Metastasis) | 6 | 0 | 6.09 | |
| 2022 | 3837 | 03, 04 (Breast, High Met vs. Breast, Non-Met) | 9 | 1 | 8.78 | |
| 2023 | 640614 | 16, 17 (Colon Tumor Tissue vs. Colon Metastasis) | 13 | 0 | 13.2 | |
| 2024 | 729531 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2025 | 729531 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2026 | 647952 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 2027 | 446913 | 15, 16 (Normal Colon vs. Colon Tumor Tissue) | 63 | 1 | 66.59 | |
| | | 18, 20 (Normal Colon Tissue vs. Colon Metastasis) | 10 | 0 | 8.55 | |
| | | 18, 19 (Normal Colon Tissue vs. Colon Tumor) | 10 | 0 | 11.43 | |
| | | 15, 17 (Normal Colon Tissue vs. Colon Metastasis) | 63 | 0 | 67.62 | |
| 2028 | 2675 | 03, 04 (Breast, High Met vs. Breast, Non-Met) | 12 | 2 | 5.85 | |
| 2029 | 643481 | 15, 17 (Normal Colon Tissue vs. Colon Metastasis) | 6 | 0 | 6.44 | |
| 2030 | 1345 | 21, 22 (Normal Prostate vs. Prostate Cancer) | 18 | 6 | 2.95 | |
| | | 15, 16 (Normal Colon vs. Colon Tumor Tissue) | 0 | 6 | | 5.68 |
| | | 08, 09 (Lung, High Met vs. Lung, Low Met) | 44 | 27 | 2.28 | |
| | | 03, 04 (Breast, High Met vs. Breast, Non-Met) | 25 | 11 | 2.22 | |
| 2031 | 26 | 03, 04 (Breast, High Met vs. Breast, Non-Met) | 62 | 0 | 60.49 | |
| | | 01, 02 (Colon, High Met vs. Colon, Low Met) | 0 | 28 | | 30.36 |
| 2032 | 945 | 01, 02 (Colon, High Met vs. Colon, Low Met) | 10 | 21 | | 2.28 |
| 2033 | 449169 | 15, 16 (Normal Colon vs. Colon Tumor Tissue) | 16 | 2 | 8.46 | |
| 2034 | 394193 | 16, 17 (Colon Tumor Tissue vs. Colon Metastasis) | 0 | 6 | | 5.91 |
| 2035 | 452212 | 15, 16 (Normal Colon vs. Colon Tumor Tissue) | 6 | 0 | 6.34 | |
| 2036 | 394193 | 16, 17 (Colon Tumor Tissue vs. Colon Metastasis) | 0 | 6 | | 5.91 |
| 2037 | 1310 | 03, 04 (Breast, High Met vs. Breast, Non-Met) | 42 | 16 | 2.56 | |
| | | 21, 22 (Normal Prostate vs. Prostate Cancer) | 15 | 2 | 7.38 | |
| 2038 | 734094 | 16, 17 (Colon Tumor Tissue vs. Colon Metastasis) | 8 | 0 | 8.12 | |
| | | 23, 24 (Normal Lung Tissue vs. Lung Tumor Tissue) | 6 | 0 | 6.06 | |
| | | 15, 16 (Normal Colon vs. Colon Tumor Tissue) | 0 | 8 | | 7.57 |
| 2039 | 646579 | 15, 17 (Normal Colon Tissue vs. Colon Metastasis) | 6 | 0 | 6.44 | |
| 2040 | 4471 | 03, 04 (Breast, High Met vs. Breast, Non-Met) | 10 | 1 | 9.76 | |
| 2041 | 729173 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 2042 | 450323 | 15, 17 (Normal Colon Tissue vs. Colon Metastasis) | 0 | 8 | | 7.45 |
| 2043 | 4652 | 03, 04 (Breast, High Met vs. Breast, Non-Met) | 16 | 2 | 7.81 | |
| 2044 | 553316 | 15, 16 (Normal Colon vs. Colon Tumor Tissue) | 12 | 3 | 4.23 | |
| 2045 | 642604 | 15, 17 (Normal Colon Tissue vs. Colon Metastasis) | 10 | 0 | 10.73 | |
| | | 16, 17 (Colon Tumor Tissue vs. Colon Metastasis) | 13 | 0 | 13.2 | |
| 2046 | 553316 | 15, 16 (Normal Colon vs. Colon Tumor Tissue) | 12 | 3 | 4.23 | |
| 2047 | 4097 | 03, 04 (Breast, High Met vs. Breast, Non-Met) | 20 | 43 | | 2.2 |
| 2048 | 6818 | 15, 17 (Normal Colon Tissue vs. Colon Metastasis) | 24 | 8 | 3.22 | |
| | | 15, 16 (Normal Colon vs. Colon Tumor Tissue) | 24 | 10 | 2.54 | |
| 2049 | 395341 | 15, 16 (Normal Colon vs. Colon Tumor Tissue) | 0 | 7 | | 6.62 |
| 2050 | 649143 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 2051 | 649143 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 2052 | 648310 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 2053 | 447574 | 15, 17 (Normal Colon Tissue vs. Colon Metastasis) | 7 | 0 | 7.51 | |
| 2054 | 648931 | 15, 17 (Normal Colon Tissue vs. Colon Metastasis) | 9 | 0 | 9.66 | |
| 2055 | 6878 | 03, 04 (Breast, High Met vs. Breast, Non-Met) | 2 | 11 | | 5.64 |
| 2056 | 452238 | 15, 16 (Normal Colon vs. Colon Tumor Tissue) | 0 | 6 | | 5.68 |
| 2057 | 1870 | 01, 02 (Colon, High Met vs. Colon, Low Met) | 11 | 31 | | 3.06 |
| | | 03, 04 (Breast, High Met vs. Breast, Non-Met) | 12 | 3 | 3.9 | |
| 2058 | 559259 | 15, 16 (Normal Colon vs. Colon Tumor Tissue) | 17 | 5 | 3.59 | |
| | | 16, 17 (Colon Tumor Tissue vs. Colon Metastasis) | 5 | 21 | | 4.14 |
| 2059 | 453457 | 16, 17 (Colon Tumor Tissue vs. Colon Metastasis) | 1 | 8 | | 7.88 |
| 2060 | 8868 | 08, 09 (Lung, High Met vs. Lung, Low Met) | 5 | 0 | 6.99 | |
| | | 03, 04 (Breast, High Met vs. Breast, Non-Met) | 9 | 1 | 8.78 | |
| 2061 | 453059 | 15, 17 (Normal Colon Tissue vs. Colon Metastasis) | 9 | 0 | 9.66 | |
| | | 15, 16 (Normal Colon vs. Colon Tumor Tissue) | 9 | 1 | 9.51 | |
| 2062 | 236368 | 18, 19 (Normal Colon Tissue vs. Colon Tumor) | 9 | 1 | 10.29 | |
| | | 15, 17 (Normal Colon Tissue vs. Colon Metastasis) | 271 | 0 | 290.88 | |
| | | 18, 20 (Normal Colon Tissue vs. Colon Metastasis) | 9 | 0 | 7.69 | |
| | | 15, 16 (Normal Colon vs. Colon Tumor Tissue) | 271 | 16 | 17.9 | |
| | | 16, 17 (Colon Tumor Tissue vs. Colon Metastasis) | 16 | 0 | 16.25 | |
| 2063 | 453059 | 15, 16 (Normal Colon vs. Colon Tumor Tissue) | 9 | 1 | 9.51 | |
| | | 15, 17 (Normal Colon Tissue vs. Colon Metastasis) | 9 | 0 | 9.66 | |
| 2064 | 549979 | 16, 17 (Colon Tumor Tissue vs. Colon Metastasis) | 0 | 7 | | 6.89 |
| 2065 | 515631 | 15, 16 (Normal Colon vs. Colon Tumor Tissue) | 0 | 6 | | 5.68 |
| 2066 | 2235 | 03, 04 (Breast, High Met vs. Breast, Non-Met) | 36 | 12 | 2.93 | |
| 2067 | 448193 | 15, 16 (Normal Colon vs. Colon Tumor Tissue) | 1 | 10 | | 9.46 |
| | | 16, 17 (Colon Tumor Tissue vs. Colon Metastasis) | 10 | 2 | 5.08 | |
| 2068 | 530774 | 15, 17 (Normal Colon Tissue vs. Colon Metastasis) | 5 | 16 | | 2.98 |
| 2069 | 650204 | 15, 17 (Normal Colon Tissue vs. Colon Metastasis) | 11 | 0 | 11.81 | |
| 2070 | 644240 | 15, 17 (Normal Colon Tissue vs. Colon Metastasis) | 7 | 0 | 7.51 | |
| 2071 | 552614 | 16, 17 (Colon Tumor Tissue vs. Colon Metastasis) | 0 | 7 | | 6.89 |
| 2072 | 727331 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 2073 | 185457 | 03, 04 (Breast, High Met vs. Breast, Non-Met) | 0 | 18 | | 18.45 |
| 2074 | 454531 | 16, 17 (Colon Tumor Tissue vs. Colon Metastasis) | 1 | 9 | | 8.86 |
| 2075 | 643485 | 15, 16 (Normal Colon vs. Colon Tumor Tissue) | 9 | 1 | 9.51 | |
| | | 15, 17 (Normal Colon Tissue vs. Colon Metastasis) | 9 | 0 | 9.66 | |
| 2076 | 733669 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2077 | 452344 | 15, 16 (Normal Colon vs. Colon Tumor Tissue) | 0 | 6 | | 5.68 |
| 2078 | 63602 | 16, 17 (Colon Tumor Tissue vs. Colon Metastasis) | 0 | 6 | | 5.91 |
| 2079 | 454155 | 15, 16 (Normal Colon vs. Colon Tumor Tissue) | 12 | 2 | 6.34 | |
| 2080 | 549903 | 16, 17 (Colon Tumor Tissue vs. Colon Metastasis) | 1 | 8 | | 7.88 |
| 2081 | 515631 | 15, 16 (Normal Colon vs. Colon Tumor Tissue) | 0 | 6 | | 5.68 |
| 2082 | 6878 | 03, 04 (Breast, High Met vs. Breast, Non-Met) | 2 | 11 | | 5.64 |
| 2083 | 2977 | 03, 04 (Breast, High Met vs. Breast, Non-Met) | 15 | 4 | 3.66 | |
| 2084 | 553823 | 15, 16 (Normal Colon vs. Colon Tumor Tissue) | 24 | 7 | 3.62 | |
| | | 15, 17 (Normal Colon Tissue vs. Colon Metastasis) | 24 | 6 | 4.29 | |
| 2085 | 3070 | 16, 17 (Colon Tumor Tissue vs. Colon Metastasis) | 0 | 6 | | 5.91 |
| 2086 | 728884 | 15, 16 (Normal Colon vs. Colon Tumor Tissue) | 0 | 8 | | 7.57 |
| | | 16, 17 (Colon Tumor Tissue vs. Colon Metastasis) | 8 | 0 | 8.12 | |
| 2087 | 8166 | 16, 17 (Colon Tumor Tissue vs. Colon Metastasis) | 6 | 0 | 6.09 | |
| 2088 | 644190 | 16, 17 (Colon Tumor Tissue vs. Colon Metastasis) | 6 | 0 | 6.09 | |
| 2089 | 733669 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2090 | 728273 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 2091 | 406499 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 2092 | 557720 | 16, 17 (Colon Tumor Tissue vs. Colon Metastasis) | 0 | 6 | | 5.91 |
| 2093 | 732050 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 2094 | 450867 | 15, 16 (Normal Colon vs. Colon Tumor Tissue) | 7 | 0 | 7.4 | |
| 2095 | 650297 | 15, 17 (Normal Colon Tissue vs. Colon Metastasis) | 6 | 0 | 6.44 | |
| 2096 | 448064 | 15, 16 (Normal Colon vs. Colon Tumor Tissue) | 11 | 3 | 3.88 | |
| 2097 | 452530 | 15, 16 (Normal Colon vs. Colon Tumor Tissue) | 10 | 2 | 5.28 | |
| 2098 | 7592 | 01, 02 (Colon, High Met vs. Colon, Low Met) | 0 | 7 | | 7.59 |
| 2099 | 733669 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2100 | 11028 | 01, 02 (Colon, High Met vs. Colon, Low Met) | 0 | 6 | | 6.5 |
| 2101 | 1013 | 01, 02 (Colon, High Met vs. Colon, Low Met) | 40 | 84 | | 2.28 |
| | | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 2102 | 549265 | 15, 16 (Normal Colon vs. Colon Tumor Tissue) | 11 | 3 | 3.88 | |
| 2103 | 376600 | 15, 16 (Normal Colon vs. Colon Tumor Tissue) | 0 | 7 | | 6.62 |
| 2104 | 643804 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 2105 | 454927 | 16, 17 (Colon Tumor Tissue vs. Colon Metastasis) | 3 | 14 | | 4.6 |
| 2106 | 446528 | 15, 16 (Normal Colon vs. Colon Tumor Tissue) | 10 | 0 | 10.57 | |
| 2107 | 2218 | 01, 02 (Colon, High Met vs. Colon, Low Met) | 2 | 21 | | 11.38 |
| | | 08, 09 (Lung, High Met vs. Lung, Low Met) | 5 | 0 | 6.99 | |
| | | 03, 04 (Breast, High Met vs. Breast, Non-Met) | 27 | 8 | 3.29 | |
| 2108 | 452704 | 15, 16 (Normal Colon vs. Colon Tumor Tissue) | 10 | 1 | 10.57 | |
| 2109 | 84895 | 16, 17 (Colon Tumor Tissue vs. Colon Metastasis) | 46 | 120 | | 2.57 |
| | | 08, 09 (Lung, High Met vs. Lung, Low Met) | 0 | 12 | | 8.59 |
| | | 15, 17 (Normal Colon Tissue vs. Colon Metastasis) | 24 | 120 | | 4.66 |
| 2110 | 157629 | 03, 04 (Breast, High Met vs. Breast, Non-Met) | 3 | 18 | | 6.15 |
| 2111 | 2930 | 01, 02 (Colon, High Met vs. Colon, Low Met) | 3 | 14 | | 5.06 |
| 2112 | 7037 | 01, 02 (Colon, High Met vs. Colon, Low Met) | 0 | 9 | | 9.76 |
| 2113 | 559806 | 16, 17 (Colon Tumor Tissue vs. Colon Metastasis) | 0 | 6 | | 5.91 |
| 2114 | 452076 | 23, 24 (Normal Lung Tissue vs. Lung Tumor Tissue) | 11 | 1 | 11.11 | |
| | | 16, 17 (Colon Tumor Tissue vs. Colon Metastasis) | 20 | 7 | 2.9 | |
| 2115 | 454869 | 16, 17 (Colon Tumor Tissue vs. Colon Metastasis) | 7 | 0 | 7.11 | |
| 2116 | 559674 | 15, 17 (Normal Colon Tissue vs. Colon Metastasis) | 0 | 7 | | 6.52 |
| 2117 | 2235 | 03, 04 (Breast, High Met vs. Breast, Non-Met) | 36 | 12 | 2.93 | |
| 2118 | 7545 | 01, 02 (Colon, High Met vs. Colon, Low Met) | 9 | 0 | 8.3 | |
| 2119 | 729173 | 15, 16 (Normal Colon vs. Colon Tumor Tissue) | 0 | 8 | | 7.57 |
| | | 16, 17 (Colon Tumor Tissue vs. Colon Metastasis) | 8 | 0 | 8.12 | |
| 2120 | 650448 | 15, 17 (Normal Colon Tissue vs. Colon Metastasis) | 7 | 0 | 7.51 | |
| 2121 | 172013 | 15, 16 (Normal Colon vs. Colon Tumor Tissue) | 6 | 0 | 6.34 | |
| 2122 | 651088 | 15, 17 (Normal Colon Tissue vs. Colon Metastasis) | 6 | 0 | 6.44 | |
| 2123 | 651088 | 15, 17 (Normal Colon Tissue vs. Colon Metastasis) | 6 | 0 | 6.44 | |
| 2124 | 726810 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 2125 | 406499 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 2126 | 556325 | 16, 17 (Colon Tumor Tissue vs. Colon Metastasis) | 1 | 8 | | 7.88 |
| 2127 | 644836 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 2128 | 649062 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 2129 | 454776 | 15, 16 (Normal Colon vs. Colon Tumor Tissue) | 5 | 20 | | 3.78 |
| 2130 | 377579 | 21, 22 (Normal Prostate vs. Prostate Cancer) | 25 | 53 | | 2.16 |
| | | 15, 17 (Normal Colon Tissue vs. Colon Metastasis) | 2 | 13 | | 6.06 |
| 2131 | 728131 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2132 | 475203 | 15, 17 (Normal Colon Tissue vs. Colon Metastasis) | 1 | 14 | | 13.04 |
| | | 15, 16 (Normal Colon vs. Colon Tumor Tissue) | 1 | 12 | | 11.35 |
| 2133 | 727314 | 16, 17 (Colon Tumor Tissue vs. Colon Metastasis) | 7 | 0 | 7.11 | |
| | | 15, 16 (Normal Colon vs. Colon Tumor Tissue) | 0 | 7 | | 6.62 |
| 2134 | 552025 | 15, 16 (Normal Colon vs. Colon Tumor Tissue) | 19 | 6 | 3.35 | |
| 2135 | 561382 | 15, 16 (Normal Colon vs. Colon Tumor Tissue) | 12 | 2 | 6.34 | |
| 2136 | 732579 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 2137 | 167 | 15, 16 (Normal Colon vs. Colon Tumor Tissue) | 7 | 19 | | 2.57 |
| 2138 | 185585 | 03, 04 (Breast, High Met vs. Breast, Non-Met) | 0 | 8 | | 8.2 |
| 2139 | 728131 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2140 | 475203 | 15, 17 (Normal Colon Tissue vs. Colon Metastasis) | 1 | 14 | | 13.04 |
| | | 15, 16 (Normal Colon vs. Colon Tumor Tissue) | 1 | 12 | | 11.35 |
| 2141 | 724616 | 16, 17 (Colon Tumor Tissue vs. Colon Metastasis) | 8 | 0 | 8.12 | |
| | | 15, 16 (Normal Colon vs. Colon Tumor Tissue) | 0 | 8 | | 7.57 |
| 2143 | 645222 | 16, 17 (Colon Tumor Tissue vs. Colon Metastasis) | 11 | 0 | 11.17 | |
| 2144 | 400362 | 16, 17 (Colon Tumor Tissue vs. Colon Metastasis) | 35 | 117 | | 3.29 |
| | | 15, 17 (Normal Colon Tissue vs. Colon Metastasis) | 21 | 117 | | 5.19 |
| 2145 | 646583 | 15, 17 (Normal Colon Tissue vs. Colon Metastasis) | 7 | 0 | 7.51 | |
| 2146 | 475203 | 15, 17 (Normal Colon Tissue vs. Colon Metastasis) | 1 | 14 | | 13.04 |
| | | 15, 16 (Normal Colon vs. Colon Tumor Tissue) | 1 | 12 | | 11.35 |
| 2147 | 550001 | 16, 17 (Colon Tumor Tissue vs. Colon Metastasis) | 10 | 2 | 5.08 | |
| 2148 | 640703 | 16, 17 (Colon Tumor Tissue vs. Colon Metastasis) | 10 | 0 | 10.16 | |
| 2149 | 646583 | 15, 17 (Normal Colon Tissue vs. Colon Metastasis) | 7 | 0 | 7.51 | |
| 2150 | 449468 | 15, 17 (Normal Colon Tissue vs. Colon Metastasis) | 2 | 11 | | 5.12 |
| 2151 | 449468 | 15, 17 (Normal Colon Tissue vs. Colon Metastasis) | 2 | 11 | | 5.12 |
| 2152 | 551628 | 15, 16 (Normal Colon vs. Colon Tumor Tissue) | 38 | 5 | 8.03 | |
| | | 15, 17 (Normal Colon Tissue vs. Colon Metastasis) | 38 | 13 | 3.14 | |
| 2153 | 449468 | 15, 17 (Normal Colon Tissue vs. Colon Metastasis) | 2 | 11 | | 5.12 |
| 2154 | 417259 | 16, 17 (Colon Tumor Tissue vs. Colon Metastasis) | 1 | 14 | | 13.79 |
| | | 15, 17 (Normal Colon Tissue vs. Colon Metastasis) | 4 | 14 | | 3.26 |
| 2155 | 448029 | 16, 17 (Colon Tumor Tissue vs. Colon Metastasis) | 10 | 1 | 10.16 | |
| 2156 | 524363 | 16, 17 (Colon Tumor Tissue vs. Colon Metastasis) | 0 | 6 | | 5.91 |
| 2157 | 446531 | 16, 17 (Colon Tumor Tissue vs. Colon Metastasis) | 0 | 6 | | 5.91 |
| 2158 | 561359 | 15, 16 (Normal Colon vs. Colon Tumor Tissue) | 12 | 2 | 6.34 | |
| | | 15, 17 (Normal Colon Tissue vs. Colon Metastasis) | 12 | 3 | 4.29 | |
| 2159 | 711297 | 15, 16 (Normal Colon vs. Colon Tumor Tissue) | 1 | 11 | | 10.41 |
| | | 16, 17 (Colon Tumor Tissue vs. Colon Metastasis) | 11 | 0 | 11.17 | |
| 2160 | 650097 | 16, 17 (Colon Tumor Tissue vs. Colon Metastasis) | 6 | 0 | 6.09 | |
| 2161 | 495715 | 16, 17 (Colon Tumor Tissue vs. Colon Metastasis) | 0 | 8 | | 7.88 |
| 2162 | 734685 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 2163 | 560515 | 16, 17 (Colon Tumor Tissue vs. Colon Metastasis) | 0 | 6 | | 5.91 |
| 2164 | 3441 | 03, 04 (Breast, High Met vs. Breast, Non-Met) | 8 | 0 | 7.81 | |
| | | 01, 02 (Colon, High Met vs. Colon, Low Met) | 4 | 13 | | 3.52 |
| 2165 | 729273 | 15, 16 (Normal Colon vs. Colon Tumor Tissue) | 0 | 7 | | 6.62 |
| | | 16, 17 (Colon Tumor Tissue vs. Colon Metastasis) | 7 | 0 | 7.11 | |
| 2166 | 557039 | 16, 17 (Colon Tumor Tissue vs. Colon Metastasis) | 2 | 16 | | 7.88 |
| | | 15, 16 (Normal Colon vs. Colon Tumor Tissue) | 11 | 2 | 5.81 | |
| 2167 | 711297 | 15, 16 (Normal Colon vs. Colon Tumor Tissue) | 1 | 11 | | 10.41 |
| | | 16, 17 (Colon Tumor Tissue vs. Colon Metastasis) | 11 | 0 | 11.17 | |
| 2168 | 711297 | 15, 16 (Normal Colon vs. Colon Tumor Tissue) | 1 | 11 | | 10.41 |
| | | 16, 17 (Colon Tumor Tissue vs. Colon Metastasis) | 11 | 0 | 11.17 | |
| 2169 | 2860 | 15, 16 (Normal Colon vs. Colon Tumor Tissue) | 1 | 13 | | 12.3 |
| | | 15, 17 (Normal Colon Tissue vs. Colon Metastasis) | 1 | 13 | | 12.11 |
| | | 03, 04 (Breast, High Met vs. Breast, Non-Met) | 1 | 48 | | 49.2 |
| | | 01, 02 (Colon, High Met vs. Colon, Low Met) | 23 | 9 | 2.36 | |
| 2170 | 558534 | 16, 17 (Colon Tumor Tissue vs. Colon Metastasis) | 0 | 7 | | 6.89 |
| 2171 | 711297 | 16, 17 (Colon Tumor Tissue vs. Colon Metastasis) | 11 | 0 | 11.17 | |
| | | 15, 16 (Normal Colon vs. Colon Tumor Tissue) | 1 | 11 | | 10.41 |
| 2172 | 378457 | 15, 16 (Normal Colon vs. Colon Tumor Tissue) | 1 | 9 | | 8.51 |
| 2173 | 646583 | 15, 17 (Normal Colon Tissue vs. Colon Metastasis) | 7 | 0 | 7.51 | |
| 2174 | 646583 | 15, 17 (Normal Colon Tissue vs. Colon Metastasis) | 7 | 0 | 7.51 | |
| 2175 | 1996 | 16, 17 (Colon Tumor Tissue vs. Colon Metastasis) | 13 | 4 | 3.3 | |
| | | 03, 04 (Breast, High Met vs. Breast, Non-Met) | 34 | 139 | | 4.19 |
| | | 15, 16 (Normal Colon vs. Colon Tumor Tissue) | 2 | 13 | | 6.15 |
| | | 08, 09 (Lung, High Met vs. Lung, Low Met) | 20 | 13 | 2.15 | |
| 2176 | 7962 | 03, 04 (Breast, High Met vs. Breast, Non-Met) | 7 | 0 | 6.83 | |
| 2177 | 645139 | 16, 17 (Colon Tumor Tissue vs. Colon Metastasis) | 9 | 0 | 9.14 | |
| 2178 | 449468 | 15, 17 (Normal Colon Tissue vs. Colon Metastasis) | 2 | 11 | | 5.12 |
| 2179 | 9898 | 03, 04 (Breast, High Met vs. Breast, Non-Met) | 4 | 14 | | 3.59 |
| 2180 | 406499 | 15, 16 (Normal Colon vs. Colon Tumor Tissue) | 6 | 0 | 6.34 | |
| | | 15, 17 (Normal Colon Tissue vs. Colon Metastasis) | 6 | 0 | 6.44 | |
| 2181 | 1257 | 01, 02 (Colon, High Met vs. Colon, Low Met) | 46 | 20 | 2.12 | |
| | | 21, 22 (Normal Prostate vs. Prostate Cancer) | 6 | 42 | | 7.12 |
| 2182 | 549903 | 16, 17 (Colon Tumor Tissue vs. Colon Metastasis) | 1 | 8 | | 7.88 |
| 2183 | 557906 | 15, 16 (Normal Colon vs. Colon Tumor Tissue) | 12 | 3 | 4.23 | |
| 2184 | 3538 | 01, 02 (Colon, High Met vs. Colon, Low Met) | 18 | 5 | 3.32 | |
| 2185 | 3114 | 03, 04 (Breast, High Met vs. Breast, Non-Met) | 9 | 1 | 8.78 | |
| 2186 | 426895 | 15, 17 (Normal Colon Tissue vs. Colon Metastasis) | 2 | 18 | | 8.38 |
| 2187 | 923 | 15, 16 (Normal Colon vs. Colon Tumor Tissue) | 7 | 23 | | 3.11 |
| | | 16, 17 (Colon Tumor Tissue vs. Colon Metastasis) | 23 | 9 | 2.6 | |
| 2188 | 645194 | 15, 17 (Normal Colon Tissue vs. Colon Metastasis) | 7 | 0 | 7.51 | |
| 2189 | 550161 | 15, 16 (Normal Colon vs. Colon Tumor Tissue) | 4 | 15 | | 3.55 |
| 2190 | 650119 | 15, 17 (Normal Colon Tissue vs. Colon Metastasis) | 7 | 0 | 7.51 | |
| 2191 | 642142 | 16, 17 (Colon Tumor Tissue vs. Colon Metastasis) | 11 | 0 | 11.17 | |
| | | 15, 16 (Normal Colon vs. Colon Tumor Tissue) | 2 | 11 | | 5.2 |
| 2192 | 419255 | 15, 17 (Normal Colon Tissue vs. Colon Metastasis) | 11 | 1 | 11.81 | |
| | | 15, 16 (Normal Colon vs. Colon Tumor Tissue) | 11 | 0 | 11.63 | |
| 2193 | 552905 | 15, 17 (Normal Colon Tissue vs. Colon Metastasis) | 0 | 7 | | 6.52 |
| 2194 | 511997 | 16, 17 (Colon Tumor Tissue vs. Colon Metastasis) | 0 | 6 | | 5.91 |
| 2195 | 551434 | 15, 16 (Normal Colon vs. Colon Tumor Tissue) | 40 | 13 | 3.25 | |
| | | 15, 17 (Normal Colon Tissue vs. Colon Metastasis) | 40 | 6 | 7.16 | |
| 2196 | 727447 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 2197 | 378786 | 16, 17 (Colon Tumor Tissue vs. Colon Metastasis) | 0 | 9 | | 8.86 |
| 2198 | 649152 | 15, 17 (Normal Colon Tissue vs. Colon Metastasis) | 6 | 0 | 6.44 | |
| 2199 | 18853 | 16, 17 (Colon Tumor Tissue vs. Colon Metastasis) | 0 | 6 | | 5.91 |
| 2200 | 643481 | 15, 17 (Normal Colon Tissue vs. Colon Metastasis) | 6 | 0 | 6.44 | |
| 2201 | 644417 | 16, 17 (Colon Tumor Tissue vs. Colon Metastasis) | 6 | 0 | 6.09 | |
| 2202 | 726788 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 2203 | 206 | 08, 09 (Lung, High Met vs. Lung, Low Met) | 19 | 4 | 6.64 | |
| | | 03, 04 (Breast, High Met vs. Breast, Non-Met) | 166 | 79 | 2.05 | |
| 2204 | 395930 | 15, 17 (Normal Colon Tissue vs. Colon Metastasis) | 6 | 0 | 6.44 | |
| 2205 | 185589 | 03, 04 (Breast, High Met vs. Breast, Non-Met) | 0 | 9 | | 9.22 |
| 2206 | 1441 | 01, 02 (Colon, High Met vs. Colon, Low Met) | 9 | 40 | | 4.82 |
| | | 03, 04 (Breast, High Met vs. Breast, Non-Met) | 38 | 16 | 2.32 | |
| 2207 | 14522 | 03, 04 (Breast, High Met vs. Breast, Non-Met) | 0 | 8 | | 8.2 |
| 2208 | 203605 | 15, 16 (Normal Colon vs. Colon Tumor Tissue) | 17 | 7 | 2.57 | |
| 2209 | 551527 | 15, 17 (Normal Colon Tissue vs. Colon Metastasis) | 0 | 7 | | 6.52 |
| 2210 | 4509 | 08, 09 (Lung, High Met vs. Lung, Low Met) | 36 | 22 | 2.29 | |
| | | 03, 04 (Breast, High Met vs. Breast, Non-Met) | 1 | 40 | | 41 |
| 2211 | 447737 | 15, 16 (Normal Colon vs. Colon Tumor Tissue) | 19 | 6 | 3.35 | |
| 2212 | 447388 | 18, 20 (Normal Colon Tissue vs. Colon Metastasis) | 18 | 0 | 15.39 | |
| | | 15, 16 (Normal Colon vs. Colon Tumor Tissue) | 142 | 2 | 75.05 | |
| | | 15, 17 (Normal Colon Tissue vs. Colon Metastasis) | 142 | 4 | 38.1 | |
| | | 18, 19 (Normal Colon Tissue vs. Colon Tumor) | 18 | 0 | 20.57 | |
| 2213 | 451932 | 15, 17 (Normal Colon Tissue vs. Colon Metastasis) | 28 | 4 | 7.51 | |
| | | 16, 17 (Colon Tumor Tissue vs. Colon Metastasis) | 15 | 4 | 3.81 | |
| 2214 | 559043 | 16, 17 (Colon Tumor Tissue vs. Colon Metastasis) | 11 | 50 | | 4.48 |
| | | 15, 16 (Normal Colon vs. Colon Tumor Tissue) | 54 | 11 | 5.19 | |
| 2215 | 380634 | 16, 17 (Colon Tumor Tissue vs. Colon Metastasis) | 3 | 14 | | 4.6 |
| 2216 | 495715 | 16, 17 (Colon Tumor Tissue vs. Colon Metastasis) | 0 | 8 | | 7.88 |
| 2217 | 451932 | 15, 17 (Normal Colon Tissue vs. Colon Metastasis) | 28 | 4 | 7.51 | |
| | | 16, 17 (Colon Tumor Tissue vs. Colon Metastasis) | 15 | 4 | 3.81 | |
| 2218 | 447939 | 15, 16 (Normal Colon vs. Colon Tumor Tissue) | 14 | 1 | 14.8 | |
| | | 16, 17 (Colon Tumor Tissue vs. Colon Metastasis) | 1 | 8 | | 7.88 |
| 2219 | 1181 | 03, 04 (Breast, High Met vs. Breast, Non-Met) | 17 | 58 | | 3.5 |
| 2220 | 376600 | 15, 16 (Normal Colon vs. Colon Tumor Tissue) | 0 | 7 | | 6.62 |
| 2221 | 234761 | 15, 17 (Normal Colon Tissue vs. Colon Metastasis) | 8 | 0 | 8.59 | |
| 2222 | 644417 | 16, 17 (Colon Tumor Tissue vs. Colon Metastasis) | 6 | 0 | 6.09 | |
| 2223 | 639048 | 15, 17 (Normal Colon Tissue vs. Colon Metastasis) | 7 | 0 | 7.51 | |
| 2224 | 11452 | 01, 02 (Colon, High Met vs. Colon, Low Met) | 0 | 6 | | 6.5 |
| 2225 | 452076 | 23, 24 (Normal Lung Tissue vs. Lung Tumor Tissue) | 11 | 1 | 11.11 | |
| | | 16, 17 (Colon Tumor Tissue vs. Colon Metastasis) | 20 | 7 | 2.9 | |
| 2226 | 644523 | 16, 17 (Colon Tumor Tissue vs. Colon Metastasis) | 6 | 0 | 6.09 | |
| 2227 | 554678 | 15, 16 (Normal Colon vs. Colon Tumor Tissue) | 10 | 2 | 5.28 | |
| 2228 | 3550 | 03, 04 (Breast, High Met vs. Breast, Non-Met) | 31 | 5 | 6.05 | |
| 2229 | 450311 | l5, 16 (Normal Colon vs. Colon Tumor Tissue) | 8 | 1 | 8.46 | |
| 2230 | 647280 | 16, 17 (Colon Tumor Tissue vs. Colon Metastasis) | 8 | 0 | 8.12 | |
| 2231 | 548858 | 16, 17 (Colon Tumor Tissue vs. Colon Metastasis) | 0 | 11 | | 10.83 |
| 2232 | 4204 | 01, 02 (Colon, High Met vs. Colon, Low Met) | 16 | 2 | 7.38 | |
| 2233 | 540690 | 23, 24 (Normal Lung Tissue vs. Lung Tumor Tissue) | 0 | 8 | | 7.92 |
| | | 16, 17 (Colon Tumor Tissue vs. Colon Metastasis) | 11 | 0 | 11.17 | |
| 2234 | 404774 | 15, 16 (Normal Colon vs. Colon Tumor Tissue) | 11 | 3 | 3.88 | |
| | | 15, 17 (Normal Colon Tissue vs. Colon Metastasis) | 11 | 1 | 11.81 | |
| 2235 | 557823 | 16, 17 (Colon Tumor Tissue vs. Colon Metastasis) | 0 | 9 | | 8.86 |
| | | 15, 17 (Normal Colon Tissue vs. Colon Metastasis) | 0 | 9 | | 8.38 |
| 2236 | 1458 | 03, 04 (Breast, High Met vs. Breast, Non-Met) | 20 | 3 | 6.5 | |
| 2237 | 485431 | 15, 16 (Normal Colon vs. Colon Tumor Tissue) | 0 | 6 | | 5.68 |
| 2238 | 2245 | 03, 04 (Breast, High Met vs. Breast, Non-Met) | 13 | 1 | 12.68 | |
| | | 01, 02 (Colon, High Met vs. Colon, Low Met) | 12 | 27 | | 2.44 |
| 2239 | 3242 | 01, 02 (Colon, High Met vs. Colon, Low Met) | 2 | 17 | | 9.22 |
| 2240 | 648747 | 15, 17 (Normal Colon Tissue vs. Colon Metastasis) | 6 | 0 | 6.44 | |
| | | 15, 16 (Normal Colon vs. Colon Tumor Tissue) | 6 | 0 | 6.34 | |
| 2241 | 3805 | 01, 02 (Colon, High Met vs. Colon, Low Met) | 17 | 3 | 5.23 | |
| 2242 | 475203 | 15, 16 (Normal Colon vs. Colon Tumor Tissue) | 1 | 12 | | 11.35 |
| | | 15, 17 (Normal Colon Tissue vs. Colon Metastasis) | 1 | 14 | | 13.04 |
| 2243 | 12018 | 03, 04 (Breast, High Met vs. Breast, Non-Met) | 0 | 6 | | 6.15 |
| 2244 | 475203 | 15, 16 (Normal Colon vs. Colon Tumor Tissue) | 1 | 12 | | 11.35 |
| | | 15, 17 (Normal Colon Tissue vs. Colon Metastasis) | 1 | 14 | | 13.04 |
| 2245 | 3805 | 01, 02 (Colon, High Met vs. Colon, Low Met) | 17 | 3 | 5.23 | |
| 2246 | 496132 | 15, 16 (Normal Colon vs. Colon Tumor Tissue) | 6 | 0 | 6.34 | |
| 2247 | 650600 | 15, 17 (Normal Colon Tissue vs. Colon Metastasis) | 6 | 0 | 6.44 | |
| 2248 | 650749 | 15, 17 (Normal Colon Tissue vs. Colon Metastasis) | 7 | 0 | 7.51 | |
| 2249 | 223148 | 15, 17 (Normal Colon Tissue vs. Colon Metastasis) | 1 | 9 | | 8.38 |
| 2250 | 449 | 15, 17 (Normal Colon Tissue vs. Colon Metastasis) | 3 | 14 | | 4.35 |
| | | 15, 16 (Normal Colon vs. Colon Tumor Tissue) | 3 | 14 | | 4.42 |
| 2251 | 735620 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2252 | 650600 | 15, 17 (Normal Colon Tissue vs. Colon Metastasis) | 6 | 0 | 6.44 | |
| 2253 | 218 | 15, 17 (Normal Colon Tissue vs. Colon Metastasis) | 1 | 11 | | 10.25 |
| | | 03, 04 (Breast, High Met vs. Breast, Non-Met) | 127 | 49 | 2.53 | |
| 2254 | 4161 | 08, 09 (Lung, High Met vs. Lung, Low Met) | 31 | 2 | 21.66 | |
| | | 01, 02 (Colon, High Met vs. Colon, Low Met) | 13 | 1 | 11.99 | |
| 2255 | 373202 | 15, 17 (Normal Colon Tissue vs. Colon Metastasis) | 5 | 22 | | 4.1 |
| 2256 | 724339 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2257 | 113291 | 15, 17 (Normal Colon Tissue vs. Colon Metastasis) | 2 | 15 | | 6.99 |
| 2258 | 736753 | 15, 16 (Normal Colon vs. Colon Tumor Tissue) | 0 | 11 | | 10.41 |
| | | 16, 17 (Colon Tumor Tissue vs. Colon Metastasis) | 11 | 0 | 11.17 | |
| 2259 | 650600 | 15, 17 (Normal Colon Tissue vs. Colon Metastasis) | 6 | 0 | 6.44 | |
| 2260 | 451569 | 15, 17 (Normal Colon Tissue vs. Colon Metastasis) | 0 | 8 | | 7.45 |
| 2261 | 1297 | 03, 04 (Breast, High Met vs. Breast, Non-Met) | 30 | 14 | 2.09 | |
| 2263 | 63602 | 16, 17 (Colon Tumor Tissue vs. Colon Metastasis) | 0 | 6 | | 5.91 |
| 2264 | 2757 | 03, 04 (Breast, High Met vs. Breast, Non-Met) | 4 | 16 | | 4.1 |
| 2265 | 373128 | 08, 09 (Lung, High Met vs. Lung, Low Met) | 4 | 29 | | 5.19 |
| | | 15, 17 (Normal Colon Tissue vs. Colon Metastasis) | 4 | 14 | | 3.26 |
| 2266 | 641479 | 16, 17 (Colon Tumor Tissue vs. Colon Metastasis) | 8 | 0 | 8.12 | |
| 2267 | 450380 | 15, 17 (Normal Colon Tissue vs. Colon Metastasis) | 4 | 31 | | 7.22 |
| | | 15, 16 (Normal Colon vs. Colon Tumor Tissue) | 4 | 23 | | 5.44 |
| 2268 | 133512 | 15, 17 (Normal Colon Tissue vs. Colon Metastasis) | 0 | 7 | | 6.52 |
| | | 18, 19 (Normal Colon Tissue vs. Colon Tumor) | 0 | 8 | | 7 |
| 2269 | 447211 | 18, 20 (Normal Colon Tissue vs. Colon Metastasis) | 9 | 0 | 7.69 | |
| | | 15, 17 (Normal Colon Tissue vs. Colon Metastasis) | 46 | 0 | 49.38 | |
| | | 15, 16 (Normal Colon vs. Colon Tumor Tissue) | 46 | 0 | 48.62 | |
| | | 18, 19 (Normal Colon Tissue vs. Colon Tumor) | 9 | 0 | 10.29 | |
| 2270 | 645222 | 16, 17 (Colon Tumor Tissue vs. Colon Metastasis) | 11 | 0 | 11.17 | |
| 2271 | 645222 | 16, 17 (Colon Tumor Tissue vs. Colon Metastasis) | 11 | 0 | 11.17 | |
| 2272 | 17372 | 15, 17 (Normal Colon Tissue vs. Colon Metastasis) | 7 | 0 | 7.51 | |
| 2273 | 451619 | 15, 16 (Normal Colon vs. Colon Tumor Tissue) | 24 | 9 | 2.82 | |
| | | 16, 17 (Colon Tumor Tissue vs. Colon Metastasis) | 9 | 39 | | 4.27 |
| 2274 | 2510 | 03, 04 (Breast, High Met vs. Breast, Non-Met) | 13 | 1 | 12.68 | |
| 2275 | 643974 | 15, 16 (Normal Colon vs. Colon Tumor Tissue) | 16 | 3 | 5.64 | |
| | | 15, 17 (Normal Colon Tissue vs. Colon Metastasis) | 16 | 0 | 17.17 | |
| 2276 | 500630 | 16, 17 (Colon Tumor Tissue vs. Colon Metastasis) | 6 | 0 | 6.09 | |
| 2277 | 3101 | 01, 02 (Colon, High Met vs. Colon, Low Met) | 4 | 19 | | 5.15 |
| 2278 | 446938 | 15, 16 (Normal Colon vs. Colon Tumor Tissue) | 8 | 1 | 8.46 | |
| 2279 | 554469 | 15, 17 (Normal Colon Tissue vs. Colon Metastasis) | 24 | 3 | 8.59 | |
| | | 15, 16 (Normal Colon vs. Colon Tumor Tissue) | 24 | 8 | 3.17 | |
| 2280 | 554469 | 15, 17 (Normal Colon Tissue vs. Colon Metastasis) | 24 | 3 | 8.59 | |
| | | 15, 16 (Normal Colon vs. Colon Tumor Tissue) | 24 | 8 | 3.17 | |
| 2281 | 2894 | 15, 16 (Normal Colon vs. Colon Tumor Tissue) | 0 | 6 | | 5.68 |
| | | 03, 04 (Breast, High Met vs. Breast, Non-Met) | 22 | 4 | 5.37 | |
| | | 08, 09 (Lung, High Met vs. Lung, Low Met) | 5 | 20 | | 2.86 |
| 2282 | 650600 | 15, 17 (Normal Colon Tissue vs. Colon Metastasis) | 6 | 0 | 6.44 | |
| 2283 | 3101 | 01, 02 (Colon, High Met vs. Colon, Low Met) | 4 | 19 | | 5.15 |
| 2284 | 554469 | 15, 17 (Normal Colon Tissue vs. Colon Metastasis) | 24 | 3 | 8.59 | |
| | | 15, 16 (Normal Colon vs. Colon Tumor Tissue) | 24 | 8 | 3.17 | |
| 2285 | 9910 | 03, 04 (Breast, High Met vs. Breast, Non-Met) | 6 | 0 | 5.85 | |
| 2286 | 400608 | 15, 17 (Normal Colon Tissue vs. Colon Metastasis) | 21 | 7 | 3.22 | |
| 2287 | 555051 | 15, 17 (Normal Colon Tissue vs. Colon Metastasis) | 0 | 8 | | 7.45 |
| | | 15, 16 (Normal Colon vs. Colon Tumor Tissue) | 0 | 9 | | 8.51 |
| 2288 | 185400 | 16, 17 (Colon Tumor Tissue vs. Colon Metastasis) | 1 | 9 | | 8.86 |
| | | 03, 04 (Breast, High Met vs. Breast, Non-Met) | 1 | 62 | | 63.55 |
| 2289 | 3059 | 03, 04 (Breast, High Met vs. Breast, Non-Met) | 16 | 3 | 5.2 | |
| 2290 | 647185 | 15, 17 (Normal Colon Tissue vs. Colon Metastasis) | 6 | 0 | 6.44 | |
| 2291 | 1669 | 21, 22 (Normal Prostate vs. Prostate Cancer) | 0 | 34 | | 34.57 |
| | | 03, 04 (Breast, High Met vs. Breast, Non-Met) | 40 | 0 | 39.03 | |
| | | 01, 02 (Colon, High Met vs. Colon, Low Met) | 11 | 29 | | 2.86 |
| 2292 | 7158 | 03, 04 (Breast, High Met vs. Breast, Non-Met) | 6 | 0 | 5.85 | |
| 2293 | 496132 | 15, 16 (Normal Colon vs. Colon Tumor Tissue) | 6 | 0 | 6.34 | |
| 2294 | 378623 | 15, 17 (Normal Colon Tissue vs. Colon Metastasis) | 3 | 33 | | 10.25 |
| | | 15, 16 (Normal Colon vs. Colon Tumor Tissue) | 3 | 24 | | 7.57 |
| | | 08, 09 (Lung, High Met vs. Lung, Low Met) | 1 | 16 | | 11.45 |
| 2295 | 1257 | 01, 02 (Colon, High Met vs. Colon, Low Met) | 46 | 20 | 2.12 | |
| | | 21, 22 (Normal Prostate vs. Prostate Cancer) | 6 | 42 | | 7.12 |
| 2296 | 648499 | 15, 17 (Normal Colon Tissue vs. Colon Metastasis) | 10 | 0 | 10.73 | |
| | | 15, 16 (Normal Colon vs. Colon Tumor Tissue) | 10 | 2 | 5.28 | |
| 2297 | 185627 | 03, 04 (Breast, High Met vs. Breast, Non-Met) | 0 | 7 | | 7.17 |
| 2298 | 640005 | 16, 17 (Colon Tumor Tissue vs. Colon Metastasis) | 6 | 0 | 6.09 | |
| | | 15, 16 (Normal Colon vs. Colon Tumor Tissue) | 20 | 6 | 3.52 | |
| | | 15, 17 (Normal Colon Tissue vs. Colon Metastasis) | 20 | 0 | 21.47 | |
| 2299 | 553462 | 15, 16 (Normal Colon vs. Colon Tumor Tissue) | 6 | 0 | 6.34 | |
| 2300 | 649852 | 15, 17 (Normal Colon Tissue vs. Colon Metastasis) | 6 | 0 | 6.44 | |
| 2301 | 422375 | 15, 17 (Normal Colon Tissue vs. Colon Metastasis) | 2 | 11 | | 5.12 |
| 2302 | 10910 | 01, 02 (Colon, High Met vs. Colon, Low Met) | 0 | 6 | | 6.5 |
| 2303 | 2737 | 16, 17 (Colon Tumor Tissue vs. Colon Metastasis) | 3 | 14 | | 4.6 |
| | | 15, 17 (Normal Colon Tissue vs. Colon Metastasis) | 4 | 14 | | 3.26 |
| 2304 | 3438 | 01, 02 (Colon, High Met vs. Colon, Low Met) | 5 | 14 | | 3.04 |
| 2305 | 3438 | 01, 02 (Colon, High Met vs. Colon, Low Met) | 5 | 14 | | 3.04 |
| 2306 | 3763 | 01, 02 (Colon, High Met vs. Colon, Low Met) | 19 | 6 | 2.92 | |
| 2307 | 648966 | 16, 17 (Colon Tumor Tissue vs. Colon Metastasis) | 6 | 0 | 6.09 | |
| 2308 | 724339 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2309 | 451569 | 15, 17 (Normal Colon Tissue vs. Colon Metastasis) | 0 | 8 | | 7.45 |
| 2310 | 554109 | 15, 16 (Normal Colon vs. Colon Tumor Tissue) | 15 | 2 | 7.93 | |
| | | 16, 17 (Colon Tumor Tissue vs. Colon Metastasis) | 2 | 11 | | 5.42 |
| 2311 | 380339 | 15, 16 (Normal Colon vs. Colon Tumor Tissue) | 166 | 57 | 3.08 | |
| | | 15, 17 (Normal Colon Tissue vs. Colon Metastasis) | 166 | 51 | 3.49 | |
| | | 19, 20 (Colon Tumor Tissue vs. Colon Metastasis) | 9 | 0 | 6.73 | |
| | | 18, 20 (Normal Colon Tissue vs. Colon Metastasis) | 18 | 0 | 15.39 | |
| 2312 | 729903 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 2313 | 45 | 08, 09 (Lung, High Met vs. Lung, Low Met) | 374 | 1067 | | 2.04 |
| | | 15, 16 (Normal Colon vs. Colon Tumor Tissue) | 40 | 119 | | 2.81 |
| | | 15, 17 (Normal Colon Tissue vs. Colon Metastasis) | 40 | 209 | | 4.87 |
| | | 18, 19 (Normal Colon Tissue vs. Colon Tumor) | 0 | 29 | | 25.38 |
| | | 18, 20 (Normal Colon Tissue vs. Colon Metastasis) | 0 | 11 | | 12.87 |
| | | 03, 04 (Breast, High Met vs. Breast, Non-Met) | 649 | 1876 | | 2.96 |
| 2314 | 454653 | 15, 17 (Normal Colon Tissue vs. Colon Metastasis) | 3 | 17 | | 5.28 |
| | | 16, 17 (Colon Tumor Tissue vs. Colon Metastasis) | 5 | 17 | | 3.35 |
| 2315 | 11536 | 03, 04 (Breast, High Met vs. Breast, Non-Met) | 6 | 0 | 5.85 | |
| 2316 | 373134 | 08, 09 (Lung, High Met vs. Lung, Low Met) | 2 | 45 | | 16.1 |
| | | 15, 17 (Normal Colon Tissue vs. Colon Metastasis) | 24 | 73 | | 2.83 |
| | | 16, 17 (Colon Tumor Tissue vs. Colon Metastasis) | 28 | 73 | | 2.57 |
| 2317 | 185691 | 03, 04 (Breast, High Met vs. Breast, Non-Met) | 0 | 6 | | 6.15 |
| 2318 | 234761 | 15, 17 (Normal Colon Tissue vs. Colon Metastasis) | 8 | 0 | 8.59 | |
| 2319 | 724339 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 2320 | 732740 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2321 | 35895 | 21, 22 (Normal Prostate vs. Prostate Cancer) | 6 | 19 | | 3.22 |
| | | 15, 16 (Normal Colon vs. Colon Tumor Tissue) | 1 | 13 | | 12.3 |
| | | 16, 17 (Colon Tumor Tissue vs. Colon Metastasis) | 13 | 0 | 13.2 | |
| 2322 | 133512 | 15, 17 (Normal Colon Tissue vs. Colon Metastasis) | 0 | 7 | | 6.52 |
| | | 18, 19 (Normal Colon Tissue vs. Colon Tumor) | 0 | 8 | | 7 |
| 2323 | 2974 | 01, 02 (Colon, High Met vs. Colon, Low Met) | 22 | 7 | 2.9 | |
| 2324 | 500 | 03, 04 (Breast, High Met vs. Breast, Non-Met) | 89 | 22 | 3.95 | |
| | | 01, 02 (Colon, High Met vs. Colon, Low Met) | 34 | 114 | | 3.64 |
| 2325 | 376919 | 16, 17 (Colon Tumor Tissue vs. Colon Metastasis) | 24 | 61 | | 2.5 |
| | | 21, 22 (Normal Prostate vs. Prostate Cancer) | 4 | 13 | | 3.3 |
| | | 15, 17 (Normal Colon Tissue vs. Colon Metastasis) | 21 | 61 | | 2.71 |
| 2326 | 8403 | 03, 04 (Breast, High Met vs. Breast, Non-Met) | 6 | 0 | 5.85 | |
| 2327 | 3643 | 01, 02 (Colon, High Met vs. Colon, Low Met) | 6 | 19 | | 3.43 |
| 2328 | 447211 | 15, 16 (Normal Colon vs. Colon Tumor Tissue) | 46 | 0 | 48.62 | |
| | | 15, 17 (Normal Colon Tissue vs. Colon Metastasis) | 46 | 0 | 49.38 | |
| | | 18, 19 (Normal Colon Tissue vs. Colon Tumor) | 9 | 0 | 10.29 | |
| | | 18, 20 (Normal Colon Tissue vs. Colon Metastasis) | 9 | 0 | 7.69 | |
| 2329 | 447211 | 15, 17 (Normal Colon Tissue vs. Colon Metastasis) | 46 | 0 | 49.38 | |
| | | 18, 19 (Normal Colon Tissue vs. Colon Tumor) | 9 | 0 | 10.29 | |
| | | 18, 20 (Normal Colon Tissue vs. Colon Metastasis) | 9 | 0 | 7.69 | |
| | | 15, 16 (Normal Colon vs. Colon Tumor Tissue) | 46 | 0 | 48.62 | |
| 2330 | 14929 | 08, 09 (Lung, High Met vs. Lung, Low Met) | 23 | 16 | 2.01 | |
| | | 15, 16 (Normal Colon vs. Colon Tumor Tissue) | 2 | 13 | | 6.15 |
| 2331 | 648934 | 15, 16 (Normal Colon vs. Colon Tumor Tissue) | 8 | 0 | 8.46 | |
| | | 15, 17 (Normal Colon Tissue vs. Colon Metastasis) | 8 | 0 | 8.59 | |
| 2332 | 731785 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2333 | 639908 | 15, 16 (Normal Colon vs. Colon Tumor Tissue) | 13 | 4 | 3.44 | |
| | | 15, 17 (Normal Colon Tissue vs. Colon Metastasis) | 13 | 0 | 13.95 | |
| 2334 | 344577 | 16, 17 (Colon Tumor Tissue vs. Colon Metastasis) | 28 | 2 | 14.22 | |
| | | 15, 17 (Normal Colon Tissue vs. Colon Metastasis) | 38 | 2 | 20.39 | |
| 2335 | 2906 | 01, 02 (Colon, High Met vs. Colon, Low Met) | 10 | 25 | | 2.71 |
| | | 15, 17 (Normal Colon Tissue vs. Colon Metastasis) | 0 | 7 | | 6.52 |
| | | 03, 04 (Breast, High Met vs. Breast, Non-Met) | 2 | 15 | | 7.69 |
| 2336 | 446938 | 15, 16 (Normal Colon vs. Colon Tumor Tissue) | 8 | 1 | 8.46 | |
| 2337 | 2493 | 03, 04 (Breast, High Met vs. Breast, Non-Met) | 33 | 9 | 3.58 | |
| 2338 | 38 | 15, 16 (Normal Colon vs. Colon Tumor Tissue) | 40 | 118 | | 2.79 |
| | | 15, 17 (Normal Colon Tissue vs. Colon Metastasis) | 40 | 259 | | 6.03 |
| | | 16, 17 (Colon Tumor Tissue vs. Colon Metastasis) | 118 | 259 | | 2.16 |
| | | 19, 20 (Colon Tumor Tissue vs. Colon Metastasis) | 88 | 6 | 10.97 | |
| | | 18, 19 (Normal Colon Tissue vs. Colon Tumor) | 3 | 88 | | 25.67 |
| 2339 | 13818 | 03, 04 (Breast, High Met vs. Breast, Non-Met) | 6 | 0 | 5.85 | |
| 2340 | 8371 | 01, 02 (Colon, High Met vs. Colon, Low Met) | 0 | 6 | | 6.5 |
| 2341 | 402494 | 15, 16 (Normal Colon vs. Colon Tumor Tissue) | 3 | 13 | | 4.1 |
| | | 16, 17 (Colon Tumor Tissue vs. Colon Metastasis) | 13 | 3 | 4.4 | |
| 2342 | 731785 | 16, 17 (Colon Tumor Tissue vs. Colon Metastasis) | 6 | 0 | 6.09 | |
| | | 15, 16 (Normal Colon vs. Colon Tumor Tissue) | 0 | 6 | | 5.68 |
| 2343 | 4621 | 03, 04 (Breast, High Met vs. Breast, Non-Met) | 11 | 1 | 10.73 | |
| 2344 | 9750 | 01, 02 (Colon, High Met vs. Colon, Low Met) | 0 | 6 | | 6.5 |
| 2345 | 133512 | 18, 19 (Normal Colon Tissue vs. Colon Tumor) | 0 | 8 | | 7 |
| | | 15, 17 (Normal Colon Tissue vs. Colon Metastasis) | 0 | 7 | | 6.52 |
| 2346 | 162626 | 03, 04 (Breast, High Met vs. Breast, Non-Met) | 1 | 12 | | 12.3 |
| 2347 | 730059 | 15, 16 (Normal Colon vs. Colon Tumor Tissue) | 0 | 6 | | 5.68 |
| | | 16, 17 (Colon Tumor Tissue vs. Colon Metastasis) | 6 | 0 | 6.09 | |
| 2348 | 2069 | 01, 02 (Colon, High Met vs. Colon, Low Met) | 26 | 8 | 3 | |
| | | 08, 09 (Lung, High Met vs. Lung, Low Met) | 21 | 6 | 4.89 | |
| | | 03, 04 (Breast, High Met vs. Breast, Non-Met) | 1 | 47 | | 48.17 |
| 2349 | 5868 | 03, 04 (Breast, High Met vs. Breast, Non-Met) | 10 | 2 | 4.88 | |
| 2350 | 2683 | 01, 02 (Colon, High Met vs. Colon, Low Met) | 3 | 22 | | 7.95 |
| | | 03, 04 (Breast, High Met vs. Breast, Non-Met) | 13 | 2 | 6.34 | |
| 2351 | 380409 | 16, 17 (Colon Tumor Tissue vs. Colon Metastasis) | 10 | 2 | 5.08 | |
| | | 15, 16 (Normal Colon vs. Colon Tumor Tissue) | 0 | 10 | | 9.46 |
| 2352 | 639991 | 15, 17 (Normal Colon Tissue vs. Colon Metastasis) | 6 | 0 | 6.44 | |
| 2353 | 535 | 03, 04 (Breast, High Met vs. Breast, Non-Met) | 87 | 13 | 6.53 | |
| 2354 | 14929 | 15, 16 (Normal Colon vs. Colon Tumor Tissue) | 2 | 13 | | 6.15 |
| | | 08, 09 (Lung, High Met vs. Lung, Low Met) | 23 | 16 | 2.01 | |
| 2355 | 134702 | 03, 04 (Breast, High Met vs. Breast, Non-Met) | 0 | 7 | | 7.17 |
| 2356 | 642477 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 2357 | 14929 | 08, 09 (Lung, High Met vs. Lung, Low Met) | 23 | 16 | 2.01 | |
| | | 15, 16 (Normal Colon vs. Colon Tumor Tissue) | 2 | 13 | | 6.15 |
| 2358 | 134702 | 03, 04 (Breast, High Met vs. Breast, Non-Met) | 0 | 7 | | 7.17 |
| 2359 | 185649 | 03, 04 (Breast, High Met vs. Breast, Non-Met) | 0 | 7 | | 7.17 |
| 2360 | 10702 | 03, 04 (Breast, High Met vs. Breast, Non-Met) | 0 | 15 | | 15.37 |
| 2361 | 643955 | 15, 17 (Normal Colon Tissue vs. Colon Metastasis) | 12 | 0 | 12.88 | |
| | | 15, 16 (Normal Colon vs. Colon Tumor Tissue) | 12 | 0 | 12.68 | |
| 2362 | 643955 | 15, 17 (Normal Colon Tissue vs. Colon Metastasis) | 12 | 0 | 12.88 | |
| | | 15, 16 (Normal Colon vs. Colon Tumor Tissue) | 12 | 0 | 12.68 | |
| 2363 | 4455 | 03, 04 (Breast, High Met vs. Breast, Non-Met) | 13 | 1 | 12.68 | |
| 2364 | 185567 | 03, 04 (Breast, High Met vs. Breast, Non-Met) | 0 | 9 | | 9.22 |
| 2365 | 9115 | 01, 02 (Colon, High Met vs. Colon, Low Met) | 0 | 7 | | 7.59 |
| 2366 | 14929 | 08, 09 (Lung, High Met vs. Lung, Low Met) | 23 | 16 | 2.01 | |
| | | 15, 16 (Normal Colon vs. Colon Tumor Tissue) | 2 | 13 | | 6.15 |
| 2367 | 14929 | 08, 09 (Lung, High Met vs. Lung, Low Met) | 23 | 16 | 2.01 | |
| | | 15, 16 (Normal Colon vs. Colon Tumor Tissue) | 2 | 13 | | 6.15 |
| 2368 | 4181 | 03, 04 (Breast, High Met vs. Breast, Non-Met) | 15 | 3 | 4.88 | |
| 2369 | 5206 | 03, 04 (Breast, High Met vs. Breast, Non-Met) | 7 | 0 | 6.83 | |
| 2370 | 825 | 03, 04 (Breast, High Met vs. Breast, Non-Met) | 70 | 25 | 2.73 | |
| | | 08, 09 (Lung, High Met vs. Lung, Low Met) | 13 | 3 | 6.06 | |
| 2371 | 825 | 08, 09 (Lung, High Met vs. Lung, Low Met) | 13 | 3 | 6.06 | |
| | | 03, 04 (Breast, High Met vs. Breast, Non-Met) | 70 | 25 | 2.73 | |
| 2372 | 825 | 03, 04 (Breast, High Met vs. Breast, Non-Met) | 70 | 25 | 2.73 | |
| | | 08, 09 (Lung, High Met vs. Lung, Low Met) | 13 | 3 | 6.06 | |
| 2373 | 2748 | 01, 02 (Colon, High Met vs. Colon, Low Met) | 2 | 26 | | 14.09 |
| | | 03, 04 (Breast, High Met vs. Breast, Non-Met) | 15 | 4 | 3.66 | |
| 2374 | 2748 | 03, 04 (Breast, High Met vs. Breast, Non-Met) | 15 | 4 | 3.66 | |
| | | 01, 02 (Colon, High Met vs. Colon, Low Met) | 2 | 26 | | 14.09 |
| 2375 | 2748 | 01, 02 (Colon, High Met vs. Colon, Low Met) | 2 | 26 | | 14.09 |
| | | 03, 04 (Breast, High Met vs. Breast, Non-Met) | 15 | 4 | 3.66 | |
| 2376 | 133512 | 18, 19 (Normal Colon Tissue vs. Colon Tumor) | 0 | 8 | | 7 |
| | | 15, 17 (Normal Colon Tissue vs. Colon Metastasis) | 0 | 7 | | 6.52 |
| 2377 | 2748 | 03, 04 (Breast, High Met vs. Breast, Non-Met) | 15 | 4 | 3.66 | |
| | | 01, 02 (Colon, High Met vs. Colon, Low Met) | 2 | 26 | | 14.09 |
| 2378 | 642477 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 2379 | 642477 | 15, 16 (Normal Colon vs. Colon Tumor Tissue) | 7 | 0 | 7.4 | |
| | | 15, 17 (Normal Colon Tissue vs. Colon Metastasis) | 7 | 0 | 7.51 | |
| 2380 | 2493 | 03, 04 (Breast, High Met vs. Breast, Non-Met) | 33 | 9 | 3.58 | |
| 2381 | 5796 | 08, 09 (Lung, High Met vs. Lung, Low Met) | 5 | 0 | 6.99 | |
| | | 03, 04 (Breast, High Met vs. Breast, Non-Met) | 14 | 3 | 4.55 | |
| 2382 | 3782 | 03, 04 (Breast, High Met vs. Breast, Non-Met) | 21 | 0 | 20.49 | |
| 2383 | 884 | 03, 04 (Breast, High Met vs. Breast, Non-Met) | 51 | 20 | 2.49 | |
| | | 15, 17 (Normal Colon Tissue vs. Colon Metastasis) | 1 | 10 | | 9.32 |
| | | 21, 22 (Normal Prostate vs. Prostate Cancer) | 20 | 7 | 2.81 | |
| | | 08, 09 (Lung, High Met vs. Lung, Low Met) | 10 | 63 | | 4.51 |
| 2384 | 5860 | 03, 04 (Breast, High Met vs. Breast, Non-Met) | 10 | 2 | 4.88 | |
| 2385 | 5275 | 01, 02 (Colon, High Met vs. Colon, Low Met) | 11 | 2 | 5.07 | |
| 2386 | 3932 | 03, 04 (Breast, High Met vs. Breast, Non-Met) | 13 | 1 | 12.68 | |
| 2387 | 884 | 08, 09 (Lung, High Met vs. Lung, Low Met) | 10 | 63 | | 4.51 |
| | | 15, 17 (Normal Colon Tissue vs. Colon Metastasis) | 1 | 10 | | 9.32 |
| | | 03, 04 (Breast, High Met vs. Breast, Non-Met) | 51 | 20 | 2.49 | |
| | | 21, 22 (Normal Prostate vs. Prostate Cancer) | 20 | 7 | 2.81 | |
| 2388 | 4455 | 03, 04 (Breast, High Met vs. Breast, Non-Met) | 13 | 1 | 12.68 | |
| 2389 | 5860 | 03, 04 (Breast, High Met vs. Breast, Non-Met) | 10 | 2 | 4.88 | |
| 2390 | 5860 | 03, 04 (Breast, High Met vs. Breast, Non-Met) | 10 | 2 | 4.88 | |
| 2391 | 372791 | 15, 17 (Normal Colon Tissue vs. Colon Metastasis) | 12 | 0 | 12.88 | |
| 2392 | 5206 | 03, 04 (Breast, High Met vs. Breast, Non-Met) | 7 | 0 | 6.83 | |
| 2393 | 372791 | 15, 17 (Normal Colon Tissue vs. Colon Metastasis) | 12 | 0 | 12.88 | |
| 2394 | 2846 | 01, 02 (Colon, High Met vs. Colon, Low Met) | 8 | 19 | | 2.57 |
| 2395 | 5275 | 01, 02 (Colon, High Met vs. Colon, Low Met) | 11 | 2 | 5.07 | |
| 2396 | 2846 | 01, 02 (Colon, High Met vs. Colon, Low Met) | 8 | 19 | | 2.57 |

We claim:

1. An isolated polynucleotide comprising a nucleotide sequence which hybridizes under stringent conditions to a sequence selected from the group consisting of SEQ ID NOS:1-2396.

2. An isolated polynucleotide comprising at least 15 contiguous nucleotides of a nucleotide sequence having at least 90% sequence identity to a sequence selected from the group consisting of: SEQ ID NOS:1-2396, a degenerate variant of SEQ ID NOS:1-2396, an antisense of SEQ ID NOS:1-2396, and a complement of SEQ ID NOS:1-2396.

3. A polynucleotide comprising a nucleotide sequence of an insert contained in a clone deposited as clone number xx of ATCC Deposit Number xx.

4. An isolated cDNA obtained by the process of amplification using a polynucleotide comprising at least 15 contiguous nucleotides of a nucleotide sequence of a sequence selected from the group consisting of SEQ ID NOS:1-2396.

5. The isolated cDNA of claim 4, wherein amplification is by polymerase chain reaction (PCR) amplification.

6. An isolated recombinant host cell containing the polynucleotide according to claims 1, 2, 3, or 4.

7. An isolated vector comprising the polynucleotide according to claims 1, 2, 3, or 4.

8. A method for producing a polypeptide, the method comprising the steps of:
culturing a recombinant host cell containing the polynucleotide according to claims 1, 2, 3, or 4, said culturing being under conditions suitable for the expression of an encoded polypeptide;
recovering the polypeptide from the host cell culture.

9. An isolated polypeptide encoded by the polynucleotide according to claims 1, 2, 3, or 4.

10. An antibody that specifically binds the polypeptide of claim 9.

11. A method of detecting differentially expressed genes correlated with a cancerous state of a mammalian cell, the method comprising the step of:
detecting at least one differentially expressed gene product in a test sample derived from a cell suspected of being cancerous, where the gene product is encoded by a gene comprising an identifying sequence of at least one of SEQ ID NOS:1-2396;
wherein detection of the differentially expressed gene product is correlated with a cancerous state of the cell from which the test sample was derived.

12. A library of polynucleotides, wherein at least one of the polynucleotides comprises the sequence information of the polynucleotide according to claims 1, 2, 3, or 4

13. The library of claim 12, wherein the library is provided on a nucleic acid array.

14. The library of claim 12, wherein the library is provided in a computer-readable format.

15. A method of inhibiting tumor growth by modulating expression of a gene product, the gene product being encoded by a gene identified by a sequence selected from the group consisting of SEQ ID NOS:1-2396.
